(19) United States
(12) Patent Application Publication
(10) Pub. No.: US 2008/0221132 A1
(43) Pub. Date: Sep. 11, 2008

(54) Multi-Functional Small Molecules as Anti-Proliferative Agents

(76) Inventors: Xiong Cai, Belmont, MA (US); Changgeng Qian, Wayland, MA (US); Stephen Gould, San Carlos, CA (US); Haixiao Zhai, Bedford, MA (US)

Correspondence Address: ELMORE PATENT LAW GROUP, PC, 515 Groton Road, Westford, MA, 01886, US

(21) Appl. No.: 11/852,458

(22) Filed: Sep. 10, 2007

Related U.S. Application Data
(60) Provisional application No. 60/843,590, filed on Sep. 11, 2006.
(60) Provisional application No. 60/895,889, filed on Mar. 20, 2007.

(51) Int. Cl. A61K 31/52; C07D 239/72; A61K 31/517; A61P 25/00; A61K 31/40; C07D 209/02; C07D 487/04; C07D 241/02; A61K 31/4965; C07D 473/36; A61K 31/519; A61K 31/505; C07D 401/02; C07D 213/02; A61K 31/44

(52) U.S. Cl. 514/263.24; 544/283; 514/266.1; 514/418; 548/465; 544/280; 514/265.1; 514/273; 544/328; 546/300; 514/351; 544/276; 544/400; 514/252.12

(57) ABSTRACT

The present invention relates to the compositions, methods, and applications of a novel approach to selective inhibition of several cellular or molecular targets with a single small molecule. More specifically, the present invention relates to multi-functional small molecules wherein one functionality is capable of inhibiting histone deacetylases (HDAC) and the other functionality is capable of inhibiting a different cellular or molecular pathway involved in aberrant cell proliferation, differentiation or survival.

### RELATED APPLICATIONS

This application claims the benefit of U.S. Provisional Application No. 60/843,590, filed on Sep. 11, 2006 and U.S. Provisional Application No. 60/895,889, filed on Mar. 20, 2007. The entire teachings of the above applications are incorporated herein by reference.

### BACKGROUND OF THE INVENTION

Elucidation of the complex and multifactorial nature of various diseases that involve multiple pathogenic pathways and numerous molecular components suggests that multi-targeted therapies may be advantageous over mono-therapies. Recent combination therapies with two or more agents for many such diseases in the areas of oncology, infectious disease, cardiovascular disease and other complex pathologies demonstrate that this combinatorial approach may provide advantages with respect to overcoming drug resistance, reduced toxicity and, in some circumstances, a synergistic therapeutic effect compared to the individual components.

Certain cancers have been effectively treated with such a combinatorial approach; however, treatment regimes using a cocktail of cytotoxic drugs often are limited by dose limiting toxicities and drug-drug interactions. More recent advances with molecularly targeted drugs have provided new approaches to combination treatment for cancer, allowing multiple targeted agents to be used simultaneously, or combining these new therapies with standard chemotherapeutics or radiation to improve outcome without reaching dose limiting toxicities. However, the ability to use such combinations currently is limited to drugs that show compatible pharmacologic and pharmacodynamic properties. In addition, the regulatory requirements to demonstrate safety and efficacy of combination therapies can be more costly and lengthy than corresponding single agent trials. Once approved, combination strategies may also be associated with increased costs to patients, as well as decreased patient compliance owing to the more intricate dosing paradigms required.

In the field of protein and polypeptide-based therapeutics it has become commonplace to prepare conjugates or fusion proteins that contain most or all of the amino acid sequences of two different proteins/polypeptides and that retain the individual binding activities of the separate proteins/polypeptides. This approach is made possible by independent folding of the component protein domains and the large size of the conjugates that permits the components to bind their cellular targets in an essentially independent manner. Such an approach is not, however, generally feasible in the case of small molecule therapeutics, where even minor structural modifications can lead to major changes in target binding and/or the pharmacokinetic/pharmacodynamic properties of the resulting molecule.

Histone acetylation is a reversible modification, with deacetylation being catalyzed by a family of enzymes termed histone deacetylases (HDACs). HDAC's are divided into four distinct classes (J Mol Biol, 2004, 338:1, 17-31). In mammalians class I HDAC's (HDAC1-3, and HDAC8) are related to yeast RPD3 HDAC, class 2 (HDAC4-7, HDAC9 and HDAC10) related to yeast HDA1, class 4 (HDAC11), and class 3 (a distinct class encompassing the sirtuins which are related to yeast Sir2).

Csordas, Biochem. J., 1990, 286: 23-38 teaches that histones are subject to post-translational acetylation of the, ε-amino groups of N-terminal lysine residues, a reaction that is catalyzed by histone acetyl transferase (HAT1). Acetylation neutralizes the positive charge of the lysine side chain, and is thought to impact chromatin structure. Indeed, access of transcription factors to chromatin templates is enhanced by histone hyperacetylation, and enrichment in underacetylated histone H4 has been found in transcriptionally silent regions of the genome (Taunton et al., Science, 1996, 272:408-411). In the case of tumor suppressor genes, transcriptional silencing due to histone modification can lead to oncogenic transformation and cancer.

Several classes of HDAC inhibitors currently are being evaluated by clinical investigators. The first FDA approved HDAC inhibitor is Suberoylanilide hydroxamic acid (SAHA, Zolinza®) for the treatment of cutaneous T-cell lymphoma (CTCL). Other HDAC inhibitors include hydroxamic acids, cyclic peptides, benzamides, and short-chain fatty acids. Hydroxamic acid derivatives PXD101 and LAQ824, are currently in the clinical development. In the benzamide class of HDAC inhibitors, MS-275, MGCD0103 and CI-994 have reached clinical trials. Mourne et al. (Abstract #4725, AACR 2005), demonstrate that thiophenyl modification of benzamides significantly enhance HDAC inhibitory activity against HDAC1.

Use of HDAC inhibitors in combination with a wide range of molecularly targeted therapies as well as standard chemotherapeutics and radiation has been shown to produce synergistic effects. Co-treatment with SAHA significantly increased EGFR2 antibody trastuzumab-induced apoptosis of BT-474 and SKBR-3 cells and induced synergistic cytotoxic effects against the breast cancer cells (Bali, Clin. Cancer Res., 2005, 11, 3392). HDAC inhibitors, such as SAHA, have demonstrated synergistic antiproliferative and apoptotic effects when used in combination with gefitinib in head and neck cancer cell lines, including lines that are resistant to gefitinib monotherapy (Bruzzese et al., Proc. AACR, 2004). Pretreating gefitinib resistant cell lines with the HDAC inhibitor, MS-275, led to a growth-inhibitory and apoptotic effect of gefitinib similar to that seen in gefitinib-sensitive NSCLC cell lines, including those harboring EGFR mutations (Witta S. E., et al., Cancer Res, 2006, 66:2, 944-50). The HDAC inhibitor PXD101 has been shown to act synergistically to inhibit proliferation with the EGFR1 inhibitor Tarceva (erlotinib) (WO2006082428A2).

Similarly, inhibition of HDAC activity has also been shown to synergize with inhibition of angiogenesis (Kim, M S, et al., Nat Med, 2001, 7:4, 437-43; Deroanne, C F, et al., Oncogene, 2002, 21:3, 427-36). Indeed, the anti-tumor activity of the HDAC inhibitor FK228 observed in PC3 xenografts is dependent upon the repression of angiogenic factors such as VEGF and bFGF (Sasakawa et al., Biochem. Pharmacol., 2003, 66, 897). The HDAC inhibitor NVP-LAQ824 has been shown to inhibit angiogenesis and have a greater anti-tumor effect when used in combination with the vascular endothelial growth factor receptor tyrosine kinase inhibitor PTK787/ZK222584 (Qian et al., Cancer Res., 2004, 64, 66260). The increase in anti-tumor activity was associated with a down regulation of the pro-angiogenic factors angiopoietin-2, Tie-2, and survivin in endothelial cells and with down regulation of hypoxia-inducible factor 1- and VEGF expression in tumor cells. Similarly the HDAC inhibitor, LBH589, has been shown to target endothelial cells leading to a reduction in an angiogenic response (Qian et al., Clin Cancer Res, 2006, 12:2, 634-42).

Histone deacetylase inhibitors have been shown to promote Gleevec (imatinib mesylate)-mediated apoptosis in both Gleevec-sensitive and -resistant (Bcr/Abl+) human myeloid leukemia cells Yu et al., Cancer Res, 2003, 63:9, 2118-26; Nimmanapalli et al., Cancer Res 63:16, 2003, 5126-35. Similarly, strong synergy between NVP-LAQ824 and imatinib mesylate was demonstrated against the BCR/ABL-expressing myeloid leukemia cell line, K562. These compounds were minimally toxic when used alone but, in combination, resulted in a marked increase in mitochondrial damage (e.g., cytochrome c, Smac/DIABLO, and apoptosis-inducing factor release), caspase activation, and apoptosis. (Weisberg et al., Leukemia. 2004, 18, 1951).

In addition, HDAC inhibitors have been shown to synergistically block cell proliferation when used in combinations with standard chemotherapeutics including 5-FU, Topotecan, Gemcitabine, Cisplatin, Doxorubicin, Docetaxle, Tomoxifen, 5-Azacytidine, Alimta, and Irinotecan (WO2006082428A2). A combination of the HDAC inhibitor, MS-275, and the nucleoside analogue fludarabine sharply increased mitochondrial injury, caspase activation, and apoptosis in leukemia cells (Maggio, S C., et. al., Cancer Res, 2004, 64:7, 2590-600). Addition of the HDAC inhibitor SAHA and topoisomerase II inhibitors (e.g., epirubicin, doxorubicin, m-AMSA, VM-26, and teniposide) have also shown synergistic effects in terms of increased cell death (Marchion, D C., J Cell Biochem, 2004, 92:2, 223-37). Similarly HDAC inhibitors have shown synergy when combined with radiation therapy (Paoluzzi, L, Cancer Biol Ther, 2004, 3:7, 612-3; Entin-Meer, M., Mol Cancer Ther, 2005, 4:12, 1952-61; Cerna, D, Curr Top Dev Biol, 2006, 73, 173-204) further illustrating the potential synergy between HDAC's and other cancer therapeutics.

Furthermore, HDAC inhibitors have also been shown to synergize with mitogen-activated protein kinase/ERK kinase (MEK), Cyclin-dependent kinase (CDK), proteasome, HSP90, and TRAIL inhibitors (Mol. Pharmacol. 2006, 69(1), 288-98; Biochem Biophys Res Commun. 2006, 27, 339(4), 1171-7; Mol Pharmacol. 2005 67(4):1166-76; Blood, 2005, 105(4), 1768-76; Cancer Res. 2006, 66(7), 3773-81; Acta Haematol. 2006, 115(1-2), 78-90; Clin Cancer Res. 2004, 10(11), 3839-52; Oncogene 2005 24(29), 4609-23; Mol Cancer Ther. 2003, 2(12), 1273-84; Biochem Pharmacol. 2003, 66(8), 1537-45; and Mol Cancer Ther. 2005, 4(11), 1772-85).

Current therapeutic regimens of the types described above attempt to address the problem of drug resistance by the administration of multiple agents. However, the combined toxicity of multiple agents due to off-target side effects as well as drug-drug interactions often limit the effectiveness of this approach. Moreover, it often is difficult to combine compounds having differing pharmacokinetics into a single dosage form, and the consequent requirement of taking multiple medications at different time intervals leads to problems with patient compliance that can undermine the efficacy of the drug combinations. In addition, the health care costs of combination therapies may be greater than for single molecule therapies. Moreover, it may be more difficult to obtain regulatory approval of a combination therapy since the burden for demonstrating activity/safety of a combination of two agents may be greater than for a single agent. (Dancey J & Chen H, Nat. Rev. Drug Dis., 2006, 5:649). The development of novel agents that target multiple therapeutic targets selected not by virtue of cross reactivity, but through rational design will help improve patient outcome while avoiding these limitations. Thus, enormous efforts are still directed to the development of selective anti-cancer drugs as well as to new and more efficacious combinations of known anti-cancer drugs.

### SUMMARY OF THE INVENTION

The present inventors have surprisingly found, however, that single compounds can be designed and prepared that combines at least two pharmacophores, and that the compounds are active at multiple therapeutic targets and are effective for treating disease. Moreover, in some cases it has even more surprisingly been found that the compounds have enhanced activity when compared to the activities of combinations of separate molecules containing the individual activities. In other words, the combination of pharmacophores into a single molecule may provide a synergistic effect as compared to the individual pharmacophores. More specifically, it has been found that it is possible to prepare compounds that simultaneously contain a first portion of the molecule that binds zinc ions and thus permits inhibition of HDAC and/or matrix metalloproteinase (MMP) activity and at least a second portion of the molecule that permits binding to a separate and distinct target that provides therapeutic benefit.

The present invention relates to the compositions, methods, and applications of a novel approach to selective inhibition of several cellular targets with a single small molecule. More specifically, the present invention relates to multi-functional small molecules wherein one pharmacophore is functionally capable of binding zinc ions and thus inhibits zinc-dependent enzymes (e.g., histone deacetylases (HDAC) and matrix metalloproteinases (MMPs) is covalently bound to a second pharmacophore with one or more functionalities capable of inhibiting a different cellular or molecule pathway or biological function involved in aberrant proliferation, differentiation or survival of cells. Such aberrant proliferation, differentiation or survival of cells may be observed in disorders such as cancer, precancerous growths or lesions, hyperplasias, and dysplasias.

In a preferred embodiment, the zinc-binding pharmacophore inhibits HDAC and is linked to a second pharmacophore that induces apoptosis, inhibits angiogenesis, and/or inhibits aberrant proliferation.

In one embodiment, the multiple functional small molecules have a molecular weight of less than 1000 g/mol, and preferably less than 600 g/mol, and most preferably less than 550 g/mol.

In one embodiment, the second pharmacophore is selected from, but not limited to, chemical compounds that are functionally capable of inhibiting the activity of tyrosine kinase, seronine/threonine kinases, DNA methyl transferases (DNMT), proteasomes, and heat-shock proteins (HSPs), vascular endothelial growth factor receptor (VEGFR), platelet-derived growth factor receptor (PDGFR), fibroblast growth factor receptor (FGFR), mitogen-activated protein kinase (MAPK/MEK), cyclin-dependent kinase (CDK), and the phosphatidylinositol 4,5-bisphosphate-AKT-mammalian target of the rapamycin pathway [P13K-AKT (RAF, mTOR)], matrix metalloproteinase, farnesyl transferase, and apoptosis.

In a most preferred embodiment, the second pharmacophore is selected from, but not limited to, chemical compounds that are functionally capable of inhibiting the activity of DNMT, EGFR, ErbB2, ErbB3, ErbB4, HER-2, VEGFR-1, VEGFR-2, VEGFR-3Flt-3, c-kit, Abl, JAK, PDGFR-α, PDGFR-β, IGF-IR, c-Met, FGFR1, FGFR3, FGFR4, c-Ret, Src, Lyn, Yes, PKC, CDK, Erk, Merk, PI3K-Akt, mTOR, Raf, CHK, Aurora, HSP90, TRAILR, caspases, IAPs, Bcl-2, Survivin, MDM2, MDM4.

Another aspect of the invention makes available the treatment, prevention or recurrence of cancer with one or more compounds of the invention.

In one embodiment, one or more compounds of the invention maybe combined with another therapy that includes, but is not limited to, anti-neoplastic agents, immunotherapeutic agents, antibodies, adjunctive agents, device, radiation therapies, chemoprotective agents, vaccines, and/or demethylating agents.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 (a) depicts a graph of EGFR enzyme assay results, (b) depicts a graph of HDAC enzyme assay results.

FIG. 2 illustrates inhibition of HDAC and EGFR in MDA-MB-468 breast cancer cell line: (a) Ac-H4 Accumulation, (b) Ac-H3 Accumulation, (c) EGFR inhibition.

FIG. 3 shows comparative data of anti-proliferative activity against several different cancer cell lines: (a) pancreatic cancer (BxPC3), (b) NSCLC (H1703), (c) breast cancer (MDA-MB-468), (d) prostate cancer (PC3).

FIG. 4 illustrates the potency of compound 12 induction of apoptosis in cancer cells: (a) HCT-116 (colon, 24 hours), (b) SKBr3 (breast, 24 hours).

FIG. 5 shows the efficacy of compound 12 in A431 Epidermoid Tumor Xenograft Model (IP Dosing).

FIG. 6 shows the efficacy of compound 12 in H358 NSCLC Xenograft Model (2-Min IV infusion).

FIG. 7 shows the efficacy of compound 12 in H292 NSCLC Xenograft Model (2-Min IV infusion).

FIG. 8 shows the efficacy of compound 12 in BxPC3 Pancreatic Cancer Xenograft Model (2-Min IV infusion).

FIG. 9 shows the efficacy of compound 12 in PC3 Prostate Cancer Xenograft Model (2-Min IV infusion).

FIG. 10 shows the efficacy of compound 12 in HCT116 Colon Cancer Xenograft Model (2-Min IV infusion).

FIG. 11A shows the percent of change in tumor size in animals treated with compound 12 or vehicle in A549 NSCLC Xenograft model.

FIG. 11B shows the percent of change in tumor size in animals treated with Erlotinib and control in A549 NSCLC Xenograft model.

FIG. 12A shows the percent of change in tumor size in animals treated with compound 12, Erlotinib or vehicle in HPAC pancreatic cancer cells.

FIG. 12B shows the percent of change in body weight in animals treated with compound 12, Erlotinib or vehicle in HPAC pancreatic cancer cells.

FIG. 13 shows the concentration of compound 12 in plasma, lung and colon after administration of hydrochloride, citrate, sodium and tartrate salts of compound 12.

FIG. 14 shows the concentration of compound 12 in the plasma of mice administered compound 12 in 30% CAPTISOL.

FIG. 15 shows the percent change in mouse body weight after administration of an IV dose of compound 12 (25, 50, 100, 200 and 400 mg/kg) in 30% CAPTISOL.

FIG. 16 shows the percent change in mouse body weight after 7 days repeat IP dosing of compound 12 (25, 50, 100, 200 and 400 mg/kg) in 30% CAPTISOL.

FIG. 17 shows the percent change in rat body weight after administration of an IV dose of compound 12 (25, 50, 100 and 200 mg/kg) in 30% CAPTISOL.

### DETAILED DESCRIPTION OF THE INVENTION

This invention provides a novel class of agents capable of inhibiting multiple biological activities. The agents of the present invention are designed with two or more activities or functionalities, where the compound comprises a first pharmacophore that binds zinc ions and inhibits zinc-dependent enzymes such as HDAC and MMPs, and a second pharmacophore, which is covalently bound to the zinc-biding moiety, and which inhibits one or more different signaling pathways or biological functions. In one embodiment, the first pharmacophore binds to Zn⁺² and inhibits HDAC. In particular embodiments, the compounds have activities that address aberrant proliferation, differentiation and/or survival of cells. Advantageously, these new agents are tumor selective and anti-neoplastic.

Most known HDAC inhibitors contain similar essential structural features such as a zinc chelator, an aliphatic linker and a hydrophobic aromatic region. The crystal structures of various known HDAC inhibitors have been solved. Proc Natl Acad Sci USA 101:42, 15064-9 (2004); Nature 401:6749, 188-93 (1999). Based on the analysis of the binding shown in these crystal structures, the present inventors have developed a pharmacophore model of HDAC inhibitors, and this pharmacophore can be added to a variety of small molecules to generate compounds that have dual or multiple distinct activities. Given the broad anti-tumor activity of HDAC inhibitors, and their ability to act synergistically with other targeted agents, this multi-pharmacophore model should be broadly applicable to the development of small molecules for the treatment of cancer.

The general structure of these novel multi-functional agents is shown below in formula (I):

A-B—C (I)

or its geometric isomers, enantiomers, diastereomers, racemates, pharmaceutically acceptable salts, prodrugs and solvates thereof, where A is a pharmacophore of an agent that inhibits aberrant cell proliferation, differentiation or survival. In a preferred aspect of the invention, A is an anti-cancer agent, B is a linker and C is a zinc-binding moiety.

In a preferred embodiment, the zinc-binding pharmacophore inhibits HDAC and is linked to a second pharmacophore that induces apoptosis, inhibits angiogenesis, and/or inhibits aberrant proliferation. In one embodiment, the multiple functional small molecules have a molecular weight of less than 1000 g/mol, and preferably less than 600 g/mol, and most preferably less than 550 g/mol.

In one embodiment of the invention, the pharmacophores for the compounds of the invention may be chosen from large numbers of anti-cancer agents available in commercial use or in clinical or pre-clinical evaluation. These agents may affect one or more protein kinases, a number of which have been demonstrated to be proto-oncogenes. These kinases may themselves become oncogenic by over-expression or mutation. Thus, by inhibiting the protein kinase activity of these proteins the disease process may be disrupted.

In one embodiment, the second pharmacophore inhibits the enzyme DNA methyltransferase (DNMT). Aberrant DNA methylation patterns are closely associated with epigenetic mutations or epimutations, which can have the same consequences as genetic mutations. For example, many tumors show hypermethylation and concomitant silencing of tumor suppressor genes. Several developmental disorders are also associated with aberrant DNA methylation. Thus, changes in DNA methylation play an important role in developmental and proliferative diseases, particularly in tumorigenesis. Inhibition of DNA methylation, particularly by inhibition of DNMTs, more particularly DNMT1, is considered a promising strategy for treatment of proliferative diseases. Azacitidine is approved for the treatment of patients in both low- and high-risk subtypes of myelodysplastic syndrome (MDS), and decitabine is currently under review by the FDA (Christine 2006; Lewis et al, 2005). It is widely accepted that histone modification and DNA methylation are intricately interrelated, working together to determine the status of gene expression and to decide cell fate (Yoo & Jones, Nat. Rev. Drug Dis, 2006, 5, 37-). Cameron et al., disclose synergy of demethylation and histone deacetylase inhibition in the re-expression of genes silenced in cancer (Nat. Genet. 1999, 21, 103-). HDAC inhibitor TSA acts synergistically with the DNMT inhibitor 5-aza-2′-deoxycytidine to reactivate DNA methylation-silenced genes (REF). HDAC inhibitors decrease DNA methyltransferase-3B messenger RNA stability and down-regulate de novo DNA methyltransferase activity in human endometrial cells (Xiong et al., Cancer Res., 2005, 65, 2684). Combination of the DNMT inhibitor (5-aza-dC) and HDAC inhibitor (trichostatin A) induced a 300-400 fold increase in ER mRNA expression (30-40 fold for 5-aza-dC & 5 fold for TSA individually) in human ER-negative breast cancer cell lines (Yang et al. Cancer Res., 2001, 61, 7025).

In one embodiment, the second pharmacophore inhibits MAP/ERK kinase (MEK). MEK inhibitors suppress a large number of human tumor cells and markedly enhance the efficacy of HDAC inhibitors to induce apoptotic cell death (Ozaki et al., BBRC, 2006, 339, 1171). HDAC inhibitor VPA inhibits angiogenesis and increases extracellular ERK phosphorylation. PD98059, a MEK inhibitor prevented the VPA-induced ERK phosphorylation. The combination of VPA with PD98059 synergistically inhibited angiogenesis in vitro and in vivo (Michaelis et al., Cell Death Differ. 2006, 13, 446). Coadministration of HDAC inhibitor SAHA and MEK inhibitor PD184352 (or U0126) resulted in a synergistic increase in mitochondrial damage, caspase activation, and apoptosis in K562 and LAMA 84 cells (Yu et al., Leukemia 2005, 19).

In one embodiment, the second pharmacophore inhibits Cyclin-dependent kinases (CDK). A variety of genetic and epigenetic events cause universal overactivity of the cell cycle cdks in human cancer, and their inhibition can lead to both cell cycle arrest and apoptosis (Shapiro, J Clin Oncol., 2006, 24, 1770). Combined treatment of human leukemia cells with HDAC inhibitor LAQ824 and CDK inhibitor roscovitine disrupts maturation and synergistically induces apoptosis, lending further support for an anti-leukemic strategy combining novel histone deacetylase and cyclin-dependent kinase inhibitors (Rosato et al., Mol. Cancer Ther., 2005, 4, 1772). Coadministration of Cyclin-dependent kinase inhibitor flavopiridol with HDAC inhibitor suberoylanilide hydroxamide and butyrate synergistically potentiated mitochondrial damage, caspase activation, poly(ADP-ribose) polymerase degradation, and cell death in both wild type and Bcl-2- or Bcl-x(L)-overexpressing cells (U937 and HL-60) and induced a pronounced loss of clonogenicity. A strategy combining CDK and HDAC inhibitors may be effective against drug-resistant leukemia cells overexpressing Bcl-2 or Bcl-x(L). (Dasmahapatra et al., Mol. Pharmacol. 2006, 69, 288).

In one embodiment, the second pharmacophore inhibits the proteosome. Inhibition of the proteasome results in disruption of protein homeostasis within the cell that can lead to apoptosis, a phenomenon preferentially observed in malignant cells. Bortezomib (Velcade®), a first-in-class proteasome inhibitor approved as an antineoplastic agent, sensitized multiple myeloma cells to HDAC inhibitor (butyrate and suberoylanilide)-induced mitochondrial dysfunction, caspase 9, 8 and 3 activation; and polypolymerase degradation (Pei et al., Clin. Cancer Res., 2004, 10, 3839). HDAC inhibitor (depsipeptide)-induced apoptosis and mitochondrial translocation of Bax were markedly enhanced by the proteasome inhibitor bortezomib in myeloid leukemic cell lines HL-60 and K562 (Sutheesophon et al., Acta Haematol., 2006, 115, 78).

In one embodiment, the second pharmacophore promotes apoptosis of cancerous cells. Apoptosis targets that are currently being explored for cancer drug discovery include, the tumor-necrosis factor (TNF)-related apoptosis-inducing ligand (TRAIL) receptors, the BCL2 family of anti-apoptotic proteins, inhibitor of apoptosis (IAP) proteins and MDM2. The HDAC inhibitor, Suberic bishydroxamate (SBHA), sensitizes melanoma to TRAIL-induced apoptosis (Zhang et al., Biochem. Pharmacol., 2003, 66, 1537). Coadministration of TNF-related apoptosis-inducing ligand (TRAIL) with HDAC inhibitors synergistically induces apoptosis, and leads to dramatic increase in mitochondrial injury and activation of caspase cascade in human myeloid leukemia cells. (Rosato et al., Mol. Pharmacol., 2003, 2, 1273). HDAC inhibitors enhance the apoptosis-induced potential of TRAIL in leukemia cells through multiple mechanisms (Shankar et al., Int. J. Mol. Med., 2005, 16, 1125).

In one embodiment, A is a pharmacophore selected from anti-cancer compound such as, but not limited to:

1.Tyrosine Kinases
1-1 ErbB family (EGFR, ErbB2, ErbB3, ErbB4)

| Compound | Structures | Known Targets |
|---|---|---|
| Gefitinib/Iressa ® | 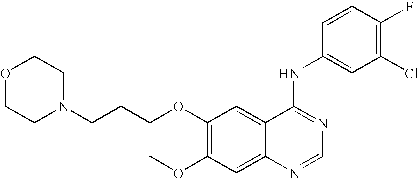 | EGFR |
| Erlotinib/Tarceva ® | 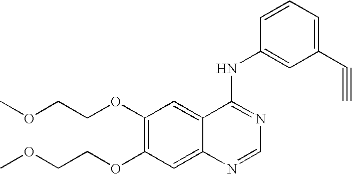 | EGFR |
| EKB-569 | 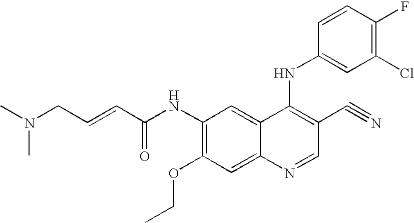 | EGFR, HER-2 |
| Lapitinib/Tykerb/GW-572016 | 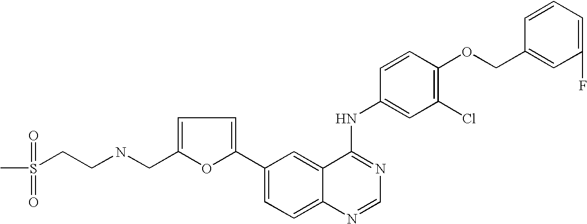 | EGFR, HER-2 |
| Canertinib/CI-1033 | 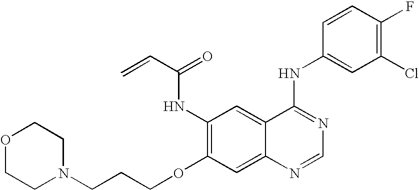 | EGFR, HER-2,ErbB3, HrbB4 |
| Mubritinib/TAK165 | 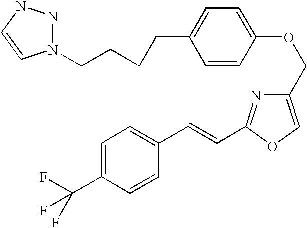 | HER-2 |
| CP-724714 | 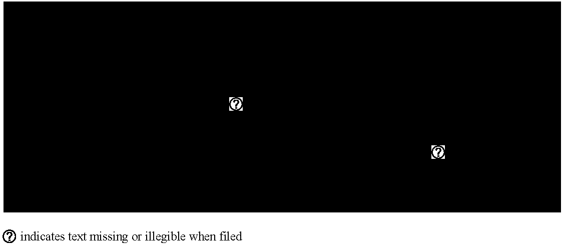 | ErbB2 |
| BIBW-2992 | 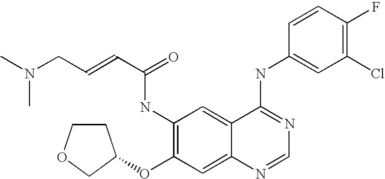 | EGFR, HER-2 |
| BMS-582664 | 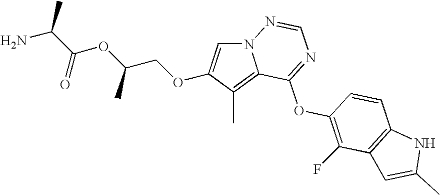 | VEGFR-2 |
| BMS-599626 | 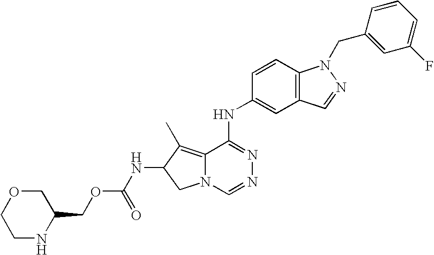 | EGFR, HER-2, |
| HKI-272 | 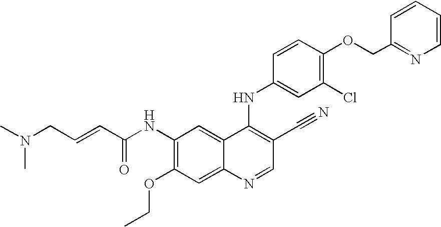 | HER-2, EGFR |
| ARRY-334543 | 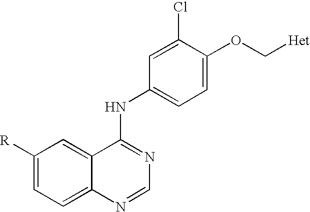 | EGFR, HER-2 |
| AV-412 | | ErbB2, EGFR |

1-2 Split kinase family

| Compound | Structures | Known Targets |
|---|---|---|
| Cediranib/AZD-2171 | 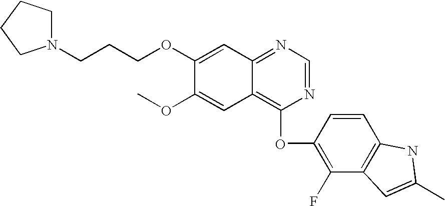 | VEGF1, VEGF2,VEGF3, Flt-1, c-Kit |
| Vatalanib/PTK787/ZK222584 | 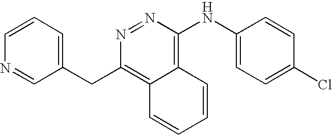 | VEGFR1, 2 & 3,PDGFR,c-Kit |
| Axitinib/AG-013736 | 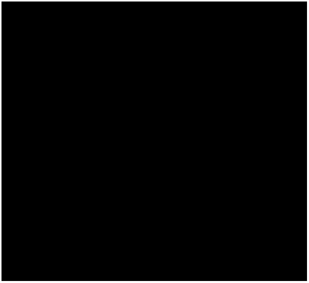 | VEGFR-1, PDGFR,VDGF-2 |
| Sunitinib/Sutent/SU11248 | 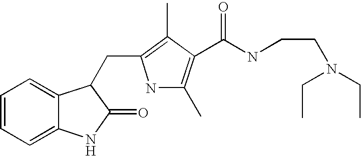 | VEGFR-2, PDGFR,FLT3, c-Kit |
| Sorafenib/Nexavar/BAY43-9006 | 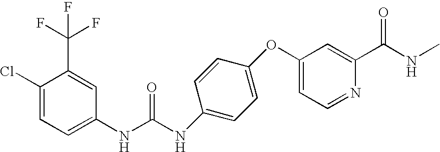 | Raf, VEGFR,PDGFR, FLT3, c-Kit, |
| BAY--57-9352 | 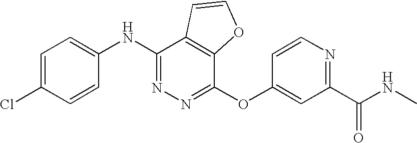 | VEGFR-2 & 3,PDGFR, c-kit |
| Pazopanib/GW-786034 | 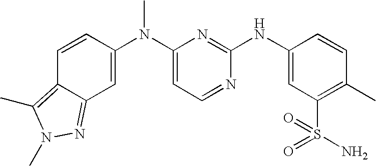 | VEGF1, VEGF2,VEGF3, PDGFR, c-Kit |
| SU6668 | 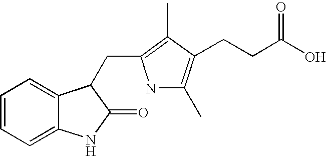 | VEGFR-2, PDGFR,FLT3, c-Kit |
| L-21649 | 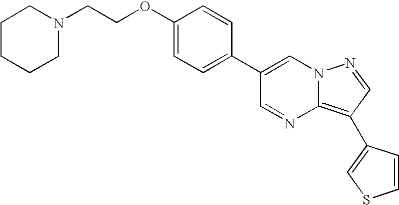 | VEGFR-2, Flt-3 |
| CP-547632 | 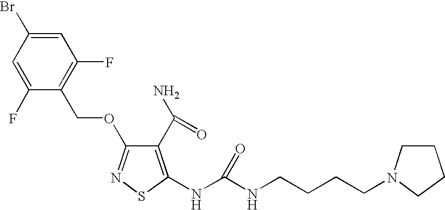 | VEGFR-2 |
| Vandetanib/AZD-6474 | 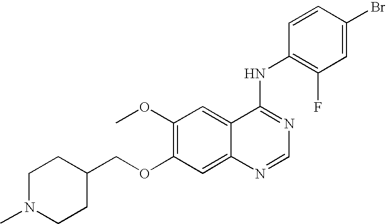 | VEGFR-2 |
| Midostaurin/PKC412 | 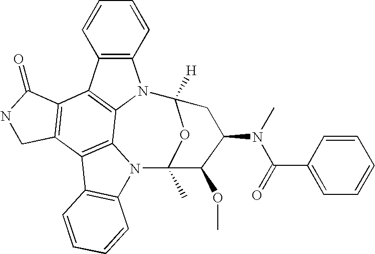 | FLT3, kit, PDGFR |
| Lestaurtinib/CEP-701 | 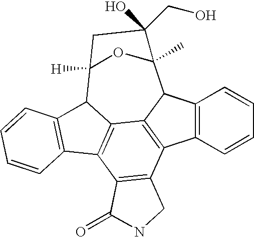 | FLT3 |
| CHIR-258/TKI-258 | 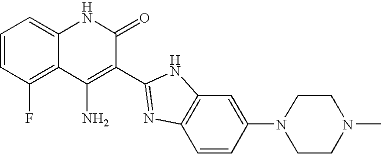 | FLT3, VEGFR, c-Kit |
| AMN107 | 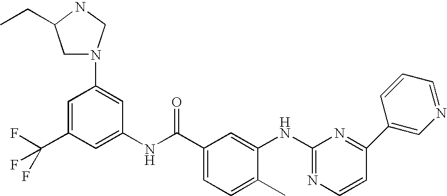 | Acr-Abl, Kit, PDGFR |
| OSI-930 | 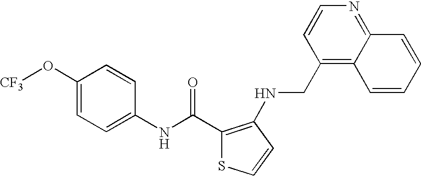 | c-Kit, VEGFR |
| Tandutinib/MLN-518/CT53518 | 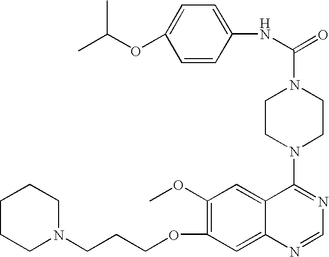 | FLT3, PDGFR, c-Kit |
| ABT-869 | 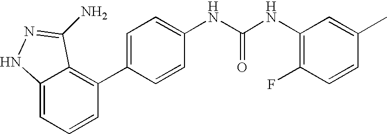 | Flt-3, KDR, VEGFR-3 |
| Imatinib/Gleevec/STI-571 | 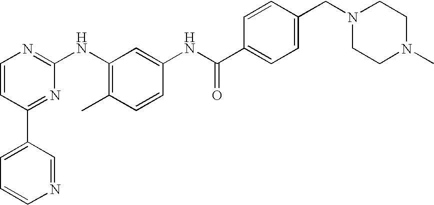 | Bcr-Abl, PDGFR, c-Kit |
| Dasatinib/BMS-354825 | 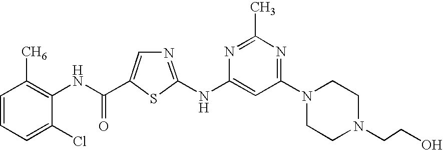 | Bcr-Abl, Src, Fyn |
| Nilotinib/AMN-107 | 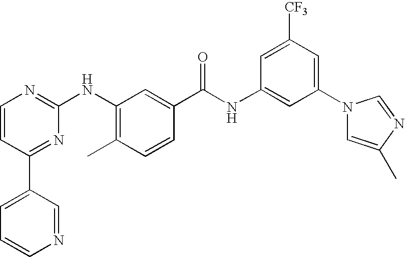 | Bcr-Abl, cKit,PDGFR |
| AZD-0530 | 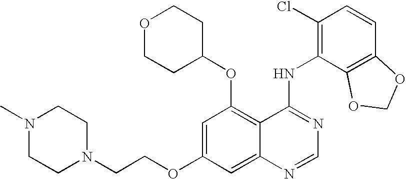 | Src, Bcr-Abl |
| Bosutinib/SKI-606 | 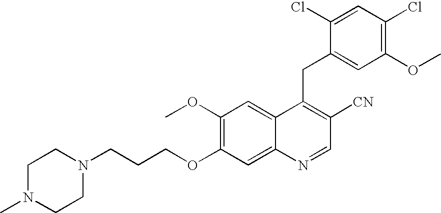 | Src, Bcr-Abl |
| AG490 | 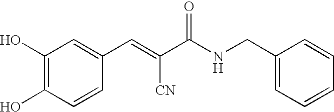 | Jak |
| AMG706 | 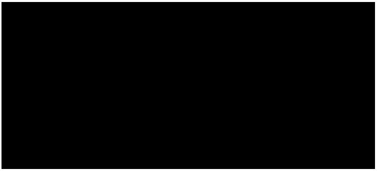 | VEGF, PDGF, Kit |
| BIBF-7055 | | VEGF-2, PDGF, Kit |
| XL999 | | FGFR, VEGFR,PDGFR, Flt3 |
| XL880 | | c-Met, VEGFR2 |
| XL647 | | EGFR, HER2,VEGFR |
| XL184 | | VEGFR2 and Met |
| XL820 | | KIT, VEGFR and PDGFR |

VEGFR family-VEGFR-1, VEGFR-2, Flt-3, c-Kit, Abl, JAK
PDGFR family-PDGFR-a, PDGFR-b, IGF-IR, c-Met
FGFR family-FGFR1, FGFR3, FGFR4, c-Ret

1-3 Combined ErbB kinase and Split kinase family (EGFR and VEGFR)

| Compound | Structures | Known Targets |
|---|---|---|
| Vandetanib/ZD6474 | 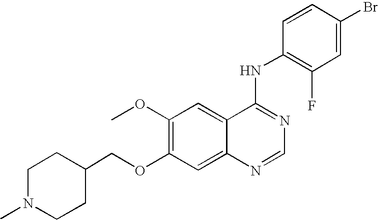 | VEGFR, EGFR |
| AEE788 | 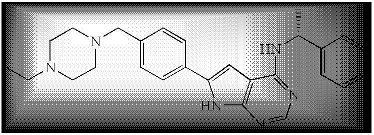 | EGFR, HerB2,VEGFR |

1-4 Src Family Kinases: Src, Lyn, Yes

| Compound | Structures | Known Targets |
|---|---|---|
| CGP76030 | 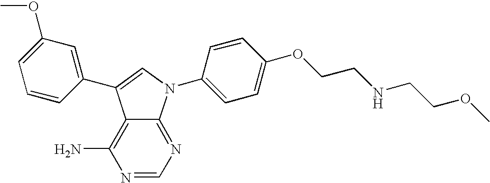 | Src, Yes, Fgr, lyn, abl |
| NVP-AAAK980 | 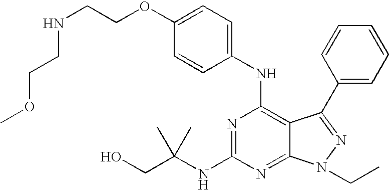 | Src |
| SU-6656 | 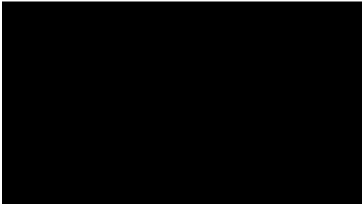 | Src |
| AZD-0530 | | Src |
| AZM-475271 | 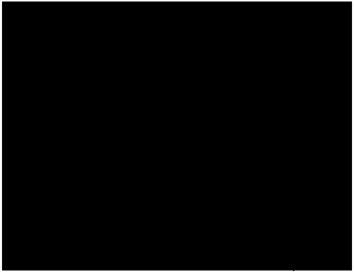 | Src |
| PP-1 | 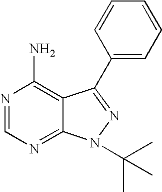 | Src,Abl |

2. Serine/threonine kinases: PKC, CDK, Erk, Mek, PI3K-Akt, mTOR, Raf, CHK, Aurora

| Compound | Structures | Known Targets |
|---|---|---|
| VX-680/MK0457 | 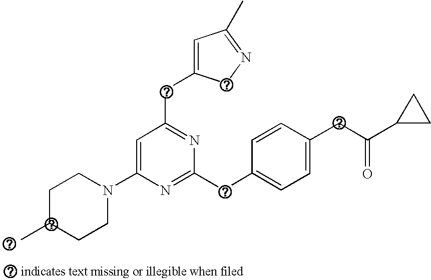 | Aurora |
| AZD-1152 | 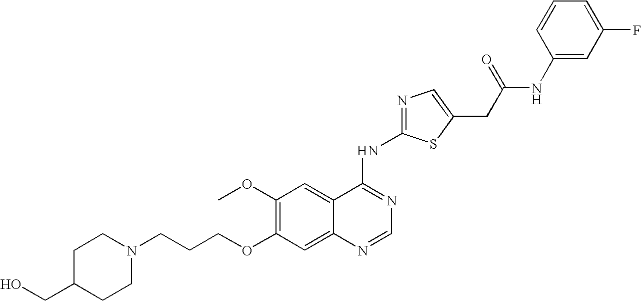 | Aurora |
| PHA-739358 | 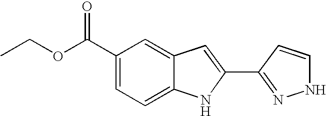 | Aurora |
| MLN-8054 | 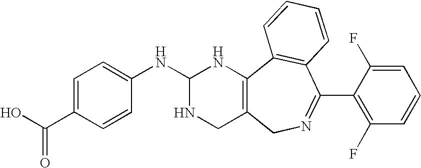 | Aurora |
| Hesperedin | 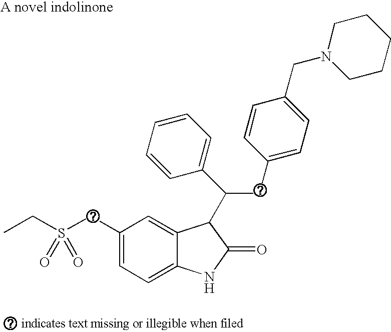 | Aurora |
| AM447439 | 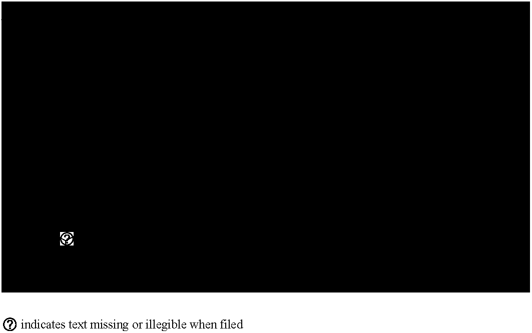 | Aurora |
| Enzastaurin/LY-317615 | 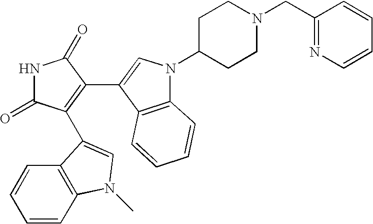 | PKC, AKT |
| Alvocidib/HMR-1275 | 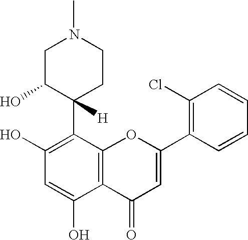 | CDK |
| AT-7519 | 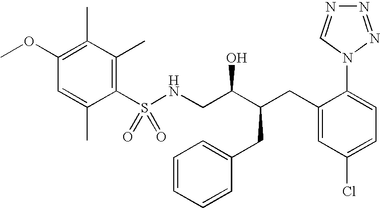 | CDK |
| UCN-01 | 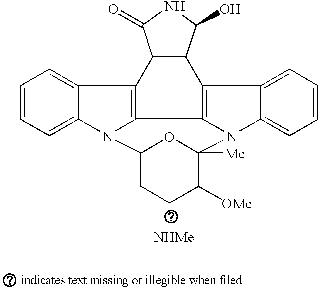 | PKC, CDK |
| CCI-779 | 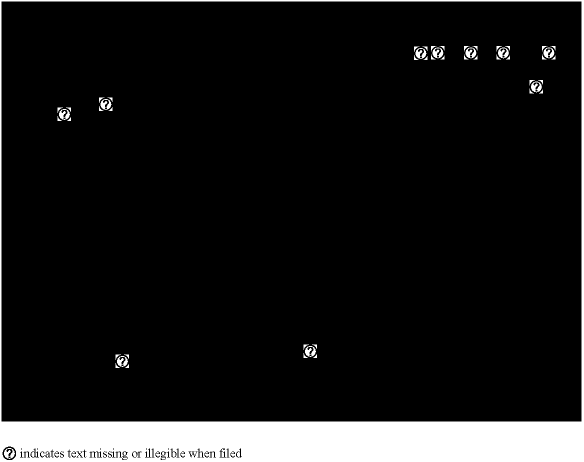 | mTOR |
| Rapamycin/sirolimus | 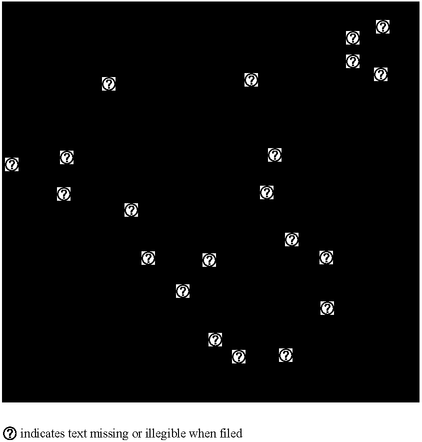 | mTOR |
| AG-024322 | 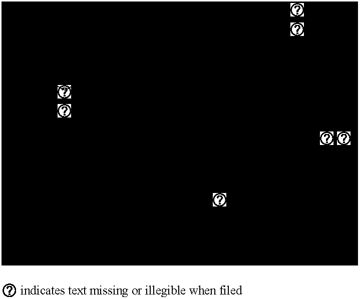 | CDK |
| BMS 387032 | 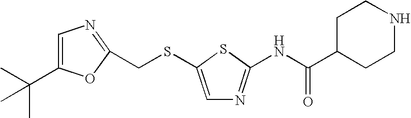 | CDK |
| R-Roscovitine/CYC202/Seliciclib | 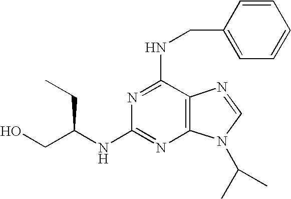 | CDK |
| PD-0332991 | 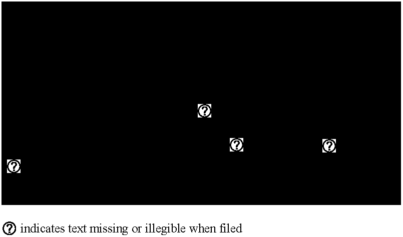 | CDK |
| SNS-032 | 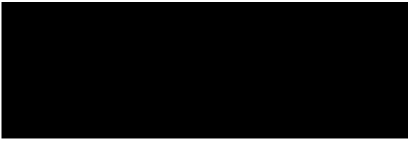 | CDK |
| RAD001/Everolimus | 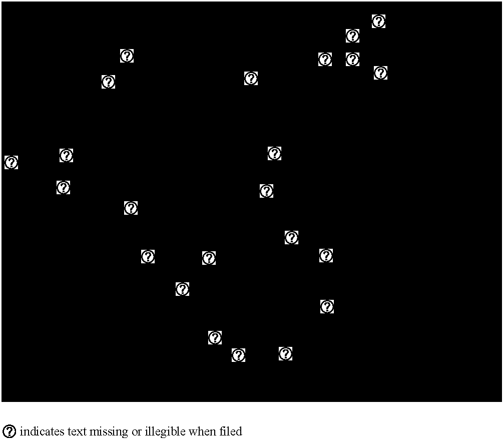 | mTOR |
| PD-0325901 | 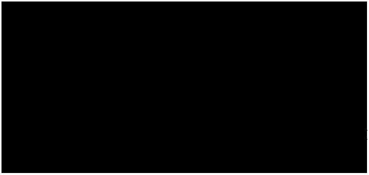 | MEK 1 & 2 |
| CI-1040/PD 184352 | 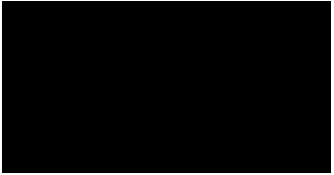 | MEK |
| AZD6244/ARRY-142886 | 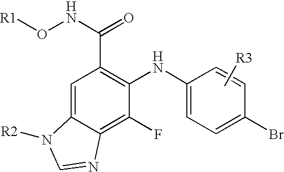 | MEK 1 & 2 |
| PI-103 | 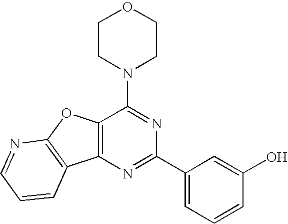 | PI-3 |
| CHIR-265 | 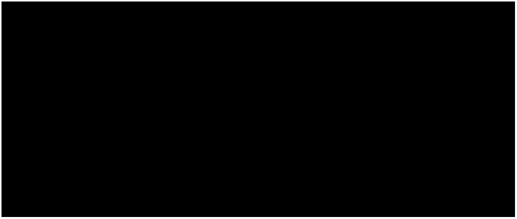 | B-Raf, VEGFR2 |

3. DNA methyl transferase (DNMT)

| | | Known |
|---|---|---|
| Compound | Structures | Targets |
| RG108 | 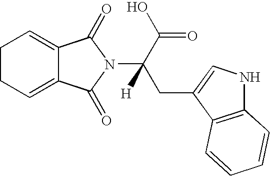 | DNMT |
| Azacitidine | 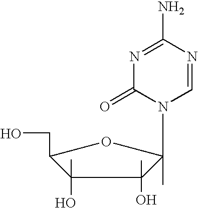 | DNMT |
| Procainamide | 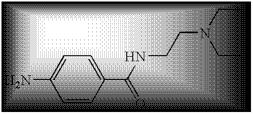 | DNMT |

4. Proteasome

| Compound | Structures | Known Targets |
|---|---|---|
| Bortezomib/Velcade | 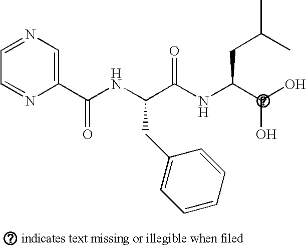 | Proteasome |
| salinosporamide A/NBI-0052 | 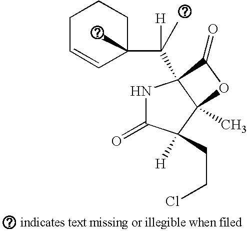 | Proteasome |
| PR-171 | 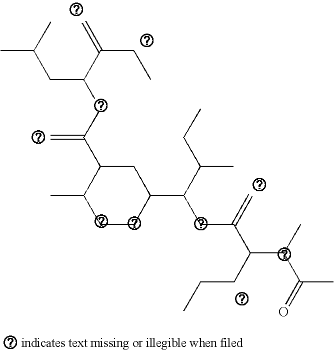 | Proteasome |

5. Matrix metalloproteinase

| Compound | Structures | Known Targets |
|---|---|---|
| COL-3 | 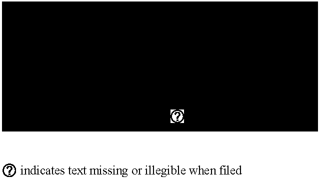 | matrix metalloproteinase inhibitor |
| BMS-275291 | 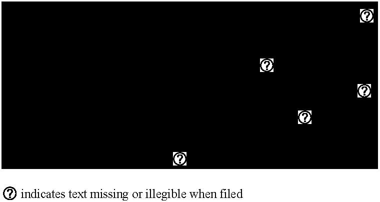 | matrix metalloproteinase inhibitor |

6. Farnesyl Transferase

| Compound | Structures | Known Targets |
|---|---|---|
| SCH66336 | 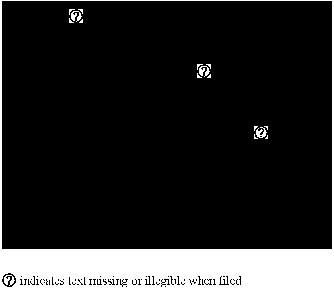 | Farnesyl Transferase |
| R115777 | 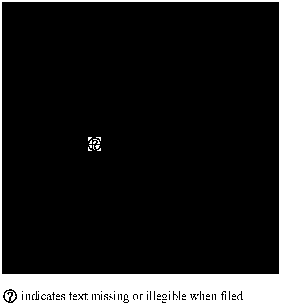 | Farnesyl Transferase |
| L778123 | 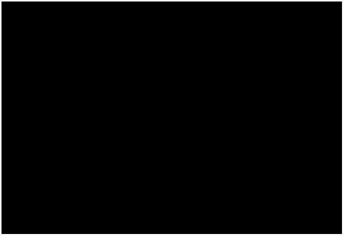 | Farnesyl Transferase |
| BMS-214662 | 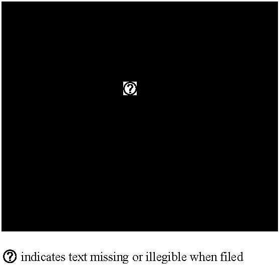 | Farnesyl Transferase |

7. Heat-shock proteins (HSPs), especially HSP90

| Compound | Structures | Known Targets |
|---|---|---|
| 17AAG/KOS-953 | 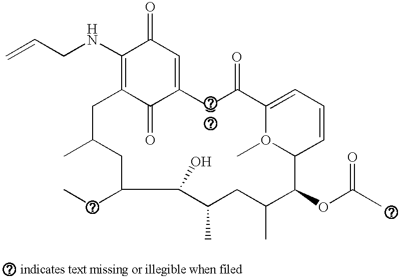 | HSP90 |
| DMAG/KOS-1022/Geldeanamycin | 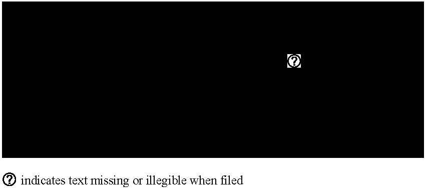 | HSP90 |
| CCT018159 | 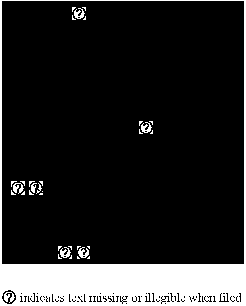 | HSP90 |
| IPI-504 | 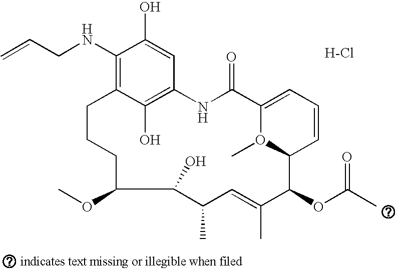 | HSP90 |
| CNF-1010 | 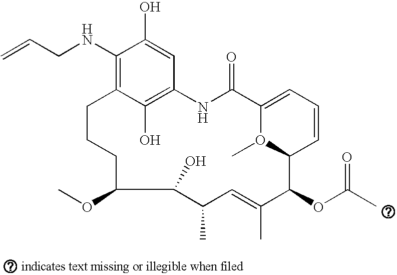 | HSP90 |
| CNF-2024 | 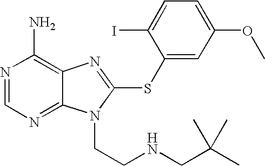 | HSP90 |

8. Apotosis agents: TRAILR, caspases, IAPs, Bcl-2, Survivin, MDM2, MDM4,

| Compound | Structures | Known Targets |
|---|---|---|
| ABT-737 | 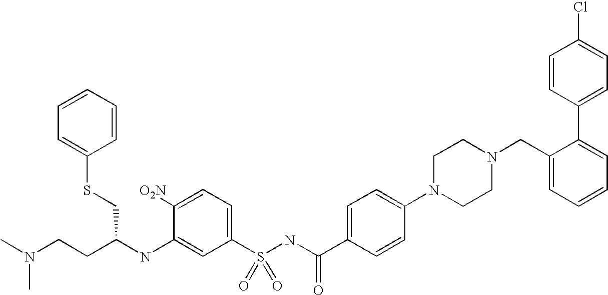 | Bcl |
| Obatoclax | 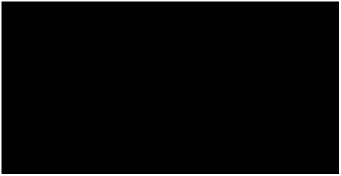 | Bcl |
| TP-115C | 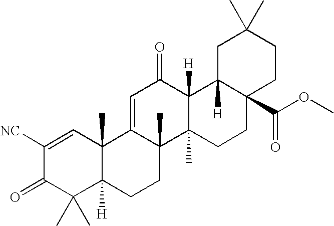 | Bcl |
| AT-101 | 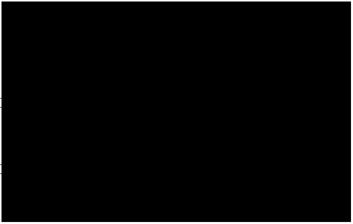 | Bcl |
| IPI-191 | | Bcl |
| JNJ-2729199 | 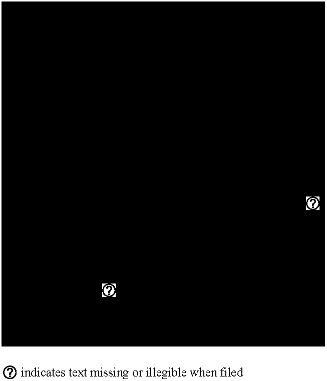 | MDM2 |
| NU-8001 | 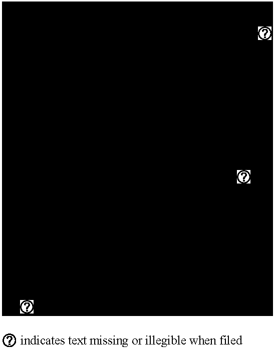 | MDM2 |
| Nutlin 2 | 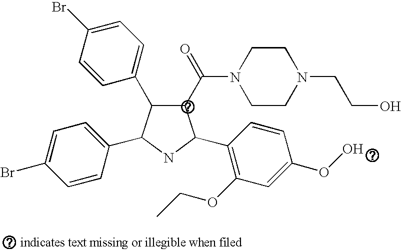 | MDM2 |
| Smac-mimetic | 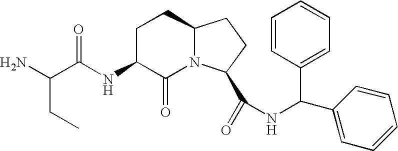 | XIAP |
| Smac-mimetic | 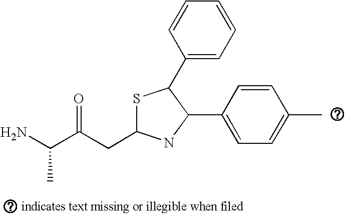 | XIAP |

In one preferred embodiment, A is a pharmacophore selected from anti-cancer compound that is characterized by having at least one nitrogen containing heterocycle or heteroaryl ring.

In one preferred embodiment, C is a zinc-binding moiety selected from:

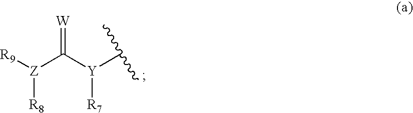

where W is O or S; Y is absent, N or CH; Z is N or CH; R₇ and R₉ are independently hydrogen, OR′, aliphatic or substituted aliphatic, wherein R′ is hydrogen, acyl, aliphatic or substituted aliphatic; provided that if R₇ and R₉ are both present, then one of R₇ or R₉ must be OR′ and if Y is absent, R₉ must be OR; and R₈ is hydrogen, acyl, aliphatic, substituted aliphatic;

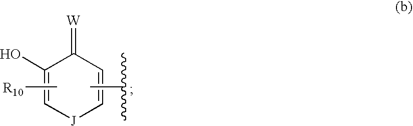

where W is O or S; J is O, NH, or NCH₃; and R₁₀ is hydrogen or lower alkyl;

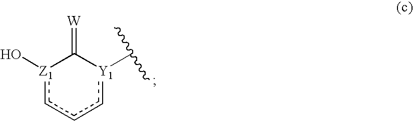

where W is O or S; Y₁ and Z₁ are independently N, C or CH; and

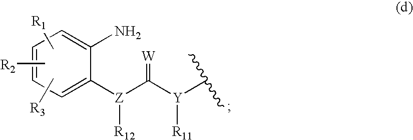

where Z, Y, and W are as previously defined; R₁₁ R₁₂ are independently selected from hydrogen or aliphatic; R₁, R₂ and R₃ are independently selected from hydrogen, hydroxy, amino, halogen, alkoxy, substituted alkoxy, alkylamino, substituted alkylamino, dialkylamino, substituted dialkylamino, substituted or unsubstituted alkylthio, substituted or unsubstituted alkylsulfonyl, CF₃, CN, NO₂, N₃, sulfonyl, acyl, aliphatic, substituted aliphatic, aryl, substituted aryl, heteroaryl, substituted heteroaryl, heterocyclic and substituted heterocyclic.

In the most preferred embodiment, C is selected from:

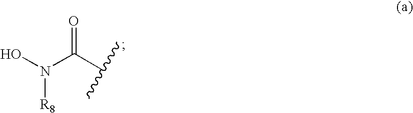

where R₈ is selected from hydrogen or lower alkyl; and

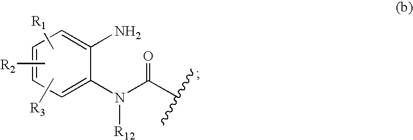

where R₁, R₂ and R₃ are independently selected from hydrogen, hydroxy, CF₃, NO₂, N₃, halogen, lower alkyl, lower alkoxy, lower alkylamino, alkoxyalkoxy (preferably methoxyethoxy), alkylaminoalkoxy (preferably methylaminoethoxy), phenyl, thiophenyl, furanyl, pyrazinyl, substituted pyrazinyl, and morpholino; and R₁₂ is selected from hydrogen or lower alkyl.

In a preferred embodiment, the bivalent B is a direct bond or straight- or branched-, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, or alkynylhereroaryl, which one or more methylenes can be interrupted or terminated by O, S, S(O), SO₂, N(R₈), C(O), substituted or unsubstituted aryl, substituted or unsubstituted heteroaryl, substituted or unsubstituted heterocyclic; such divalent B linkers include but are not limited to alkyl, alkenyl, alkynyl, alkylaryl, alkenylaryl, alkynylaryl, alkoxyaryl, alkylaminoaryl, alkoxyalkyl, alkylaminoalkyl, alkylheterocycloalkyl, alkylheteroarylalkyl, alkylamino, N(R₈)alkenyl, N(R₈)alkynyl, N(R₈)alkoxyalkyl, N(R₈)alkylaminoalkyl, N(R₈)alkylaminocarbonyl, N(R₈)alkylaryl, N(R₈)alkenylaryl, N(R₈)alkynylaryl, N(R₈)alkoxyaryl, N(R₈)alkylaminoaryl, N(R₈)cycloalkyl, N(R₈)aryl, N(R₈)heteroaryl, N(R₈)heterocycloalkyl, N(R₈)alkylheterocycloalkyl, alkoxy, O-alkenyl, O-alkynyl, O-alkoxyalkyl, O-alkylaminoalkyl, O-alkylaminocarbonyl, O-alkylaryl, O-alkenylaryl, O-alkynylaryl, O-alkoxyaryl, O-alkylaminoaryl, O-cycloalkyl, O-aryl, O-heteroaryl, O-heterocycloalkyl, O-alkylheterocycloalkyl, C(O)alkyl, C(O)-alkenyl, C(O)alkynyl, C(O)alkylaryl, C(O)alkenylaryl, C(O)alkynylaryl, C(O)alkoxyalkyl, C(O)alkylaminoalkyl, C(O)alkylaminocarbonyl, C(O)cycloalkyl, C(O)aryl, C(O)heteroaryl, C(O)heterocycloalkyl, CON(R₈), CON(R₈)alkyl, CON(R₈)alkenyl, CON(R₈)alkynyl, CON(R₈)alkylaryl, CON(R₈)alkenylaryl, CON(R₈)alkynylaryl, CON(R₈)alkoxyalkyl, CON(R₈)alkylaminoalkyl, CON(R₈)alkylaminocarbonyl, CON(R₈)alkoxyaryl, CON(R₈)alkylaminoaryl, CON(R₈)cycloalkyl, CON(R₈)aryl, CON(R₈)heteroaryl, CON(R₈)heterocycloalkyl, CON(R₈)alkylheterocycloalkyl, N(R₈)C(O)alkyl, N(R₈)C(O)alkenyl, N(R₈)C(O)— alkynyl, N(R₈)C(O)alkylaryl, N(R₈)C(O)alkenylaryl, N(R₈)C(O)alkynylaryl, N(R₈)C(O)alkoxyalkyl, N(R₈)C(O)alkylaminoalkyl, N(R₈)C(O)alkylaminocarbonyl, N(R₈)C(O)alkoxyaryl, N(R₈)C(O)alkylaminoaryl, N(R₈)C(O)cycloalkyl, N(R₈)C(O)aryl, N(R₈)C(O)heteroaryl, N(R₈)C(O)heterocycloalkyl, N(R₈)C(O)alkylheterocycloalkyl, NHC(O)NH, NHC(O)NH-alkyl, NHC(O)NH-alkenyl, NHC(O)NH-alkynyl, NHC(O)NH-alkylaryl, NHC(O)NH-alkenylaryl, NHC(O)NH-alkynylaryl, NHC(O)NH-alkoxyaryl, NHC(O)NH-alkylaminoaryl, NHC(O)NH-cycloalkyl, NHC(O)NH-aryl, NHC(O)NH-heteroaryl, NHC(O)NH-heterocycloalkyl, NHC(O)NH-alkylheterocycloalkyl, S-alkyl, S-alkenyl, S-alkynyl, S-alkoxyalkyl, S-alkylaminoalkyl, S-alkylaryl, S-alkylaminocarbonyl, S-alkylaryl, S-alkynylaryl, S-alkoxyaryl, S-alkylaminoaryl, S-cycloalkyl, S-aryl, S-heteroaryl, S-heterocycloalkyl, S-alkylheterocycloalkyl, S(O)alkyl, S(O)alkenyl, S(O)alkynyl, S(O)alkoxyalkyl, S(O)alkylaminoalkyl, S(O)alkylaminocarbonyl, S(O)alkylaryl, S(O)alkenylaryl, S(O)alkynylaryl, S(O)alkoxyaryl, S(O)alkylaminoaryl, S(O)cycloalkyl, S(O)aryl, S(O)heteroaryl, S(O)heterocycloalkyl, S(O)alkylheterocycloalkyl, S(O)₂alkyl, S(O)₂alkenyl, S(O)₂alkynyl, S(O)₂alkoxyalkyl, S(O)₂alkylaminoalkyl, S(O)₂alkylaminocarbonyl, S(O)₂alkylaryl, S(O)₂alkenylaryl, S(O)₂alkynylaryl, S(O)₂alkoxyaryl, S(O)₂alkylaminoaryl, S(O)₂cycloalkyl, S(O)₂aryl, S(O)₂heteroaryl, S(O)₂heterocycloalkyl, S(O)₂alkylheterocycloalkyl, SO₂NH, SO₂NH-alkyl, SO₂NH-alkenyl, SO₂NH-alkynyl, SO₂NH-alkylaryl, SO₂NH-alkenylaryl, SO₂NH-alkynylaryl, SO₂NH-cycloalkyl, SO₂NH-aryl, SO₂NH-heteroaryl, SO₂NH-heterocycloalkyl, SO₂NH-alkylheterocycloalkyl, alkylaryloxyalkoxy, alkylaryloxyalkylamino, alkylarylaminoalkoxy, alkylarylaminoalkylamino, alkylarylalkylaminoalkoxy, alkylarylalkylaminoalkoxy, alkenylaryloxyalkoxy, alkenylaryloxyalkylamino, alkenylarylaminoalkoxy, alkenylarylaminoalkylamino, alkenylarylalkylaminoalkoxy, alkenylarylalkylaminoalkylamino. It is understood that the alkyl, alkenyl, alkynyl, aryl, heteroaryl, cycloalkyl, heterocyclic and the like can be further substituted.

In a more preferred embodiment, B is a straight chain alkyl, alkenyl, alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, or alkynylhereroaryl. One or more methylenes can be interrupted or terminated by —O—, —N(R₈)—, —C(O)—, —C(O)N(R₈)—, or —C(O)O—. Preferably, the C group is attached to B via an aliphatic moiety within B.

In one embodiment, the linker B is between 1-24 atoms, preferably 4-24 atoms, preferably 4-18 atoms, more preferably 4-12 atoms, and most preferably about 4-10 atoms.

In the most preferred embodiment, B is selected from straight chain C1-C10 alkyl, C1-C10 alkenyl, C1-C10 alkynyl, C1-C10 alkoxy, alkoxyC1-C10alkoxy, C1-C10 alkylamino, alkoxyC1-C10alkylamino, C1-C10 alkylcarbonylamino, C1-C10 alkylaminocarbonyl, aryloxyC1-C10alkoxy, aryloxyC1-C10alkylamino, aryloxyC1-C10alkylamino carbonyl, C1-C10-alkylaminoalkylaminocarbonyl, C1-C10 alkyl(N-alkyl)aminoalkyl-aminocarbonyl, alkylaminoalkylamino, alkylcarbonylaminoalkylamino, alkyl(N-alkyl)aminoalkylamino, (N-alkyl)alkylcarbonylaminoalkylamino, alkylaminoalkyl, alkylaminoalkylaminoalkyl, alkylpiperazinoalkyl, piperazinoalkyl, alkylpiperazino, alkenylaryloxyC1-C10alkoxy, alkenylarylaminoC1-C10alkoxy, alkenylaryllalkylaminoC1-C10alkoxy, alkenylaryloxyC1-C10alkylamino, alkenylaryloxyC1-C10alkylaminocarbonyl, piperazinoalkylaryl, heteroarylC1-C10alkyl, heteroarylC2-C10alkenyl, heteroarylC2-C10alkynyl, heteroarylC1-C10alkylamino, heteroarylC1-C10alkoxy, heteroaryloxyC1-C10alkyl, heteroaryloxyC2-C10alkenyl, heteroaryloxyC2-C10alkynyl, heteroaryloxyC1-C10alkylamino, heteroaryloxyC1-C10alkoxy. In the most preferred embodiments, the C group is attached to B via an aliphatic moiety carbon chain within B.

In one embodiment, the multi-functional compounds of the present invention are compounds represented by formula (II) or its geometric isomers, enantiomers, diastereomers, racemates, pharmaceutically acceptable salts, prodrugs and solvates thereof:

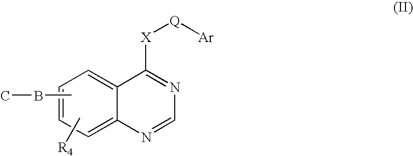

wherein
Ar is aryl, substituted aryl, heteroaryl, or substituted heteroaryl;
Q is absent or substituted or unsubstituted alkyl;
X is O, S, NH, or alkylamino;
B and C are as previously defined.

In a most preferred embodiment, Ar is phenyl, substituted phenyl, naphthyl, substituted naphthyl, pyridinyl, substituted pyridinyl, furanyl, substituted furanyl, pyrrolyl, substituted pyrrolyl; pyrazolyl, substituted pyrazolyl, oxazolyl, substituted oxazolyl, thiophenyl, or substituted thiophenyl; Q is absent or substituted or unsubstituted alkyl; X is O, S, NH, or alkylamino; R₄ is independently selected from hydrogen, hydroxy, amino, halogen, CF₃, CN, N₃, NO₂, sulfonyl, acyl, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, or alkynylheterocyclylalkynyl, which one or more methylenes can be interrupted or terminated by O, S, S(O), SO₂, N(R₈), C(O), substituted or unsubstituted aryl, substituted or unsubstituted heteroaryl, substituted or unsubstituted heterocyclic; where R₈ is hydrogen, acyl, aliphatic or substituted aliphatic; B and C and are as previously defined in the most preferred embodiment.

In one embodiment, the multi-functional compounds of the present invention are compounds represented by formula (III) as illustrated below, or its geometric isomers, enantiomers, diastereomers, racemates, pharmaceutically acceptable salts, prodrugs and solvates thereof:

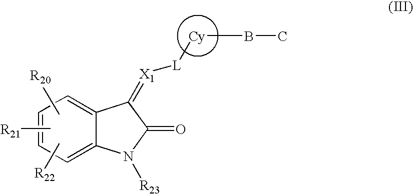

wherein
X₁ is N, CR₈; where R₈ is as previously defined;
L is absent or NH;
Cy is aryl, substituted aryl, heteroaryl, or substituted heteroaryl;
R₂₀, R₂₁, R₂₂ are independently selected from hydrogen, hydroxy, amino, halogen, alkoxy, substituted alkoxy, alkylamino, substituted alkylamino, dialkylamino, substituted dialkylamino, substituted or unsubstituted alkylthio, substituted or unsubstituted alkylsulfonyl, CF₃, CN, N₃, NO₂, sulfonyl, acyl, aliphatic, substituted aliphatic, aryl, substituted aryl, heteroaryl, substituted heteroaryl, heterocyclic, and substituted heterocyclic;
R₂₃ is hydrogen or aliphatic;
B, C, R₁, R₂, and R₃ are as previously defined.

In one embodiment, the multi-functional compounds of the present invention are compounds represented by formulae (IV) and (V) as illustrated below, or its geometric isomers, enantiomers, diastereomers, racemates, pharmaceutically acceptable salts, prodrugs and solvates thereof:

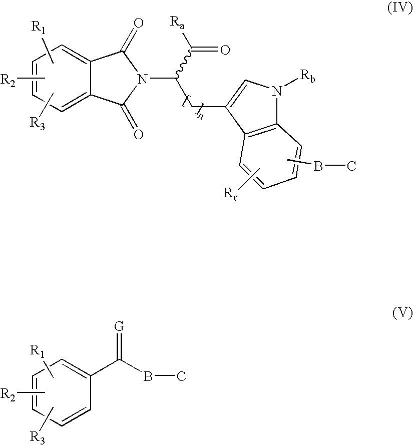

wherein
Rₐ is hydroxy, amino, alkoxy, alkylamino, dialkylamino;
R_{b} is hydrogen, aliphatic group, acyl;
R_{c} is selected from R₁;
n is 0, 1, 2, or 3;
G is S or O;
B, C and R₁, R₂, and R₃ are as previously defined.

In one embodiment, the multi-functional compounds of the present invention are compounds represented by formulae (VI) and (VII) as illustrated below, or its geometric isomers, enantiomers, diastereomers, racemates, pharmaceutically acceptable salts, prodrugs and solvates thereof:

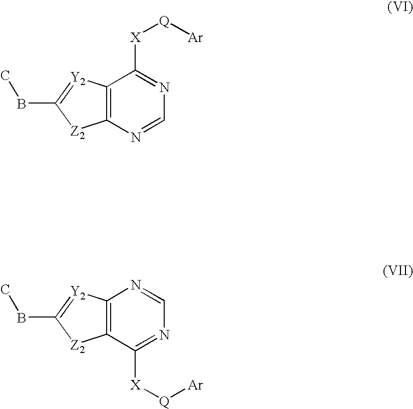

wherein
Z₂ is O, S, NH or alkylamino
Y₂ is N or CR₂₀; where R₂₀ is selected from hydrogen, halogen, aliphatic, aryl, substituted aryl, heteroaryl, substituted heteroaryl;
B, C, Q, X and Ar are as previously defined.

In one embodiment, the multi-functional compounds of the present invention are compounds represented by formulae (VIII) and (IX) as illustrated below, or its geometric isomers, enantiomers, diastereomers, racemates, pharmaceutically acceptable salts, prodrugs and solvates thereof:

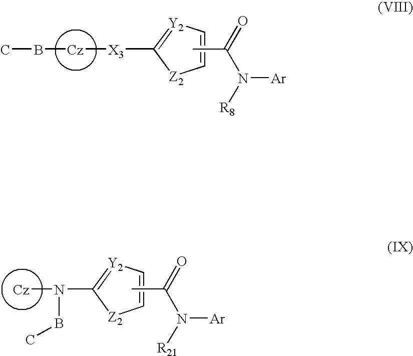

wherein
Cz is selected from aryl, substituted aryl, heteroaryl, substituted heteroaryl, and heterocylic;
X₃ is NH, alkylamino O or S;
C, B, Y₂, Z₂, Ar and R₈ are as previously defined.

In one embodiment, the multi-functional compounds of the present invention are compounds represented by formulae (X) and (XI) as illustrated below, or its geometric isomers, enantiomers, diastereomers, racemates, pharmaceutically acceptable salts, prodrugs and solvates thereof:

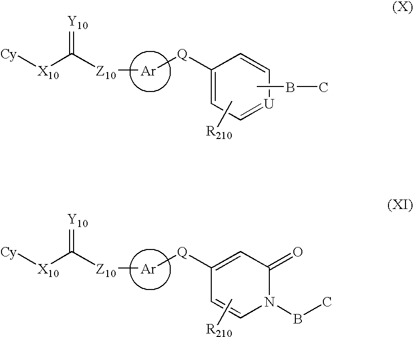

wherein
U is N, CH or C;
Ar is aryl, substituted aryl, heteroaryl, substituted heteroaryl, cycloalkyl, substituted cycloalkyl, heterocylic or substituted heterocyclic;
Q is O, S, SO, SO₂, NH, substituted or unsubstituted alkylamino, or substituted or unsubstituted C₁-C₃ alkyl;
Y₁₀ is O, S or NH;
X₁₀ and Z₁₀ are independently NH, substituted or unsubstituted alkylamino, or substituted or unsubstituted C₁-C₃ alkyl;
Cy is aryl, substituted aryl, heteroaryl, substituted heteroaryl, cycloalkyl, or heterocycloalkyl;
R₂₁₀ is independently selected from hydrogen, hydroxy, amino, halogen, substituted or unsubstituted alkoxy, substituted or unsubstituted alkylamino, substituted or unsubstituted dialkylamino, substituted or unsubstituted alkylthio, substituted or unsubstituted alkylsulfonyl, CF₃, CN, NO₂, N₃, sulfonyl, acyl, aryl, substituted aryl, heteroaryl, substituted heteroaryl, heterocyclic, substituted heterocyclic, aliphatic, and substituted aliphatic;
C and B are as previously defined.

In one embodiment, the multi-functional compounds of the present invention are compounds represented by formula (XII) as illustrated below, or its geometric isomers, enantiomers, diastereomers, racemates, pharmaceutically acceptable salts, prodrugs and solvates thereof:

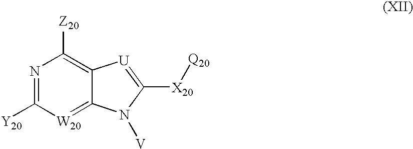

wherein
U is N or CH;
W₂₀ is N or CH;
X₂₀ is absent, O, S, S(O), S(O)₂, N(R₈), CF₂ or C₁-C₆ alkyl, C₂-C₆ alkenyl, C₂-C₆ alkynyl, in which one or more methylene can be interrupted or terminated by O, S, SO, SO₂, N(R₈), R₈ is hydrogen, acyl, aliphatic or substituted aliphatic;
Y₂₀ is independently hydrogen, halogen, NO₂, CN, or lower alkyl;
Z₂₀ is amino, alkylamino, or dialkylamino;
Q₂₀ is aryl, substituted aryl, heteroaryl, substituted heteroaryl, cycloalkyl, or heterocycloalkyl;
V is hydrogen, straight- or branched-, substituted or unsubstituted alkyl, substituted or unsubstituted alkenyl, substituted or unsubstituted alkynyl, which one or more methylenes can be interrupted or terminated by O, S, S(O), SO₂, N(R₈), C(O), substituted or unsubstituted aryl, substituted or unsubstituted heteroaryl, substituted or unsubstituted heterocyclic; substituted or unsubstituted cycloalkyl;
   and wherein Q₂₀ and/or V is further substituted by

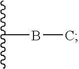

C, B and U are as previously defined.

In one preferred embodiment, C is a zinc-binding moiety selected from:

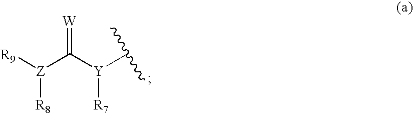

where W is O or S; Y is absent, N or CH; Z is N or CH; R₇ and R₉ are independently hydrogen, hydroxy, aliphatic group; provided that if R₇ and R₉ are both present, then one of R₇ or R₉ must be hydroxy and if Y is absent, R₉ must be hydroxy; and R₈ is hydrogen or aliphatic group;

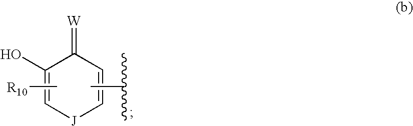

where W is O or S; J is O, NH, or NCH₃; and R₁₀ is hydrogen or lower alkyl;

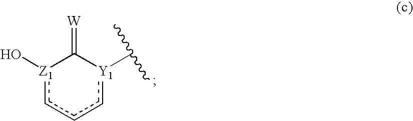

where W is O or S; Y₁ and Z₁ are independently N, C or CH; and

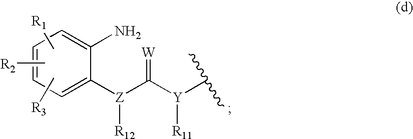

where Z, Y, and W are as previously defined; R₁₁ R₁₂ are independently selected from hydrogen or aliphatic; R₁, R₂ and R₃ are independently selected from hydrogen, hydroxy, amino, halogen, alkoxy, alkylamino, dialkylamino, CF₃, CN, NO₂, sulfonyl, acyl, aliphatic, substituted aliphatic, aryl, substituted aryl, heteroaryl, substituted heteroaryl, heterocyclic and substituted heterocyclic.

In the most preferred embodiment, C is selected from:

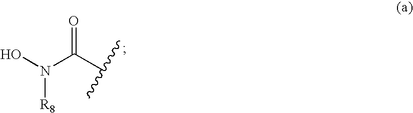

where R₈ is selected from hydrogen or lower alkyl; and

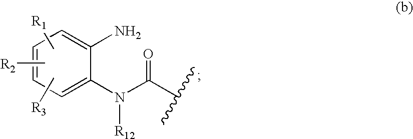

where R₁, R₂ and R₃ are independently selected from hydrogen, hydroxy, CF₃, NO₂, halogen, lower alkyl, lower alkoxy, lower alkylamino, alkoxyalkoxy (preferably methoxyethoxy), alkylaminoalkoxy (preferably methylaminoethoxy), phenyl, thiophenyl, furanyl, pyrazinyl, substituted pyrazinyl, and morpholino; and R₁₂ is selected from hydrogen or lower alkyl.

In a preferred embodiment, the bivalent B is a direct bond or straight- or branched-, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, or alkynylheterocyclylalkynyl, which one or more methylenes can be interrupted or terminated by O, S, S(O), SO₂, N(R₈), C(O), substituted or unsubstituted aryl, substituted or unsubstituted heteroaryl, substituted or unsubstituted heterocyclic; such divalent B linkers include but are not limited to alkyl, alkenyl, alkynyl, alkylaryl, alkenylaryl, alkynylaryl, alkoxyaryl, alkylaminoaryl, alkoxyalkyl, alkylaminoalkyl, alkylheterocycloalkyl, alkylheteroarylalkyl, alkylamino, N(R₈)alkenyl, N(R₈)alkynyl, N(R₈)alkoxyalkyl, N(R₈)alkylaminoalkyl, N(R₈)alkylaminocarbonyl, N(R₈)alkylaryl, N(R₈)alkenylaryl, N(R₈)alkynylaryl, N(R₈)alkoxyaryl, N(R₈)alkylaminoaryl, N(R₈)cycloalkyl, N(R₈)aryl, N(R₈)heteroaryl, N(R₈)heterocycloalkyl, N(R₈)alkylheterocycloalkyl, alkoxy, O-alkenyl, O-alkynyl, O-alkoxyalkyl, O-alkylaminoalkyl, O-alkylaminocarbonyl, O-alkylaryl, O-alkenylaryl, O-alkynylaryl, O-alkoxyaryl, O-alkylaminoaryl, O-cycloalkyl, O-aryl, O-heteroaryl, O-heterocycloalkyl, O-alkylheterocycloalkyl, C(O)alkyl, C(O)-alkenyl, C(O)alkynyl, C(O)alkylaryl, C(O)alkenylaryl, C(O)alkynylaryl, C(O)alkoxyalkyl, C(O)alkylaminoalkyl, C(O)alkylaminocarbonyl, C(O)cycloalkyl, C(O)aryl, C(O)heteroaryl, C(O)heterocycloalkyl, CON(R₈), CON(R₈)alkyl, CON(R₈)alkenyl, CON(R₈)alkynyl, CON(R₈)alkylaryl, CON(R₈)alkenylaryl, CON(R₈)alkynylaryl, CON(R₈)alkoxyalkyl, CON(R₈)alkylaminoalkyl, CON(R₈)alkylaminocarbonyl, CON(R₈)alkoxyaryl, CON(R₈)alkylaminoaryl, CON(R₈)cycloalkyl, CON(R₈)aryl, CON(R₈)heteroaryl, CON(R₈)heterocycloalkyl, CON(R₈)alkylheterocycloalkyl, N(R₈)C(O)alkyl, N(R₈)C(O)alkenyl, N(R₈)C(O)-alkynyl, N(R₈)C(O)alkylaryl, N(R₈)C(O)alkenylaryl, N(R₈)C(O)alkynylaryl, N(R₈)C(O)alkoxyalkyl, N(R₈)C(O)alkylaminoalkyl, N(R₈)C(O)alkylaminocarbonyl, N(R₈)C(O)alkoxyaryl, N(R₈)C(O)alkylaminoaryl, N(R₈)C(O)cycloalkyl, N(R₈)C(O)aryl, N(R₈)C(O)heteroaryl, N(R₈)C(O)heterocycloalkyl, N(R₈)C(O)alkylheterocycloalkyl, NHC(O)NH, NHC(O)NH-alkyl, NHC(O)NH-alkenyl, NHC(O)NH-alkynyl, NHC(O)NH-alkylaryl, NHC(O)NH-alkenylaryl, NHC(O)NH-alkynylaryl, NHC(O)NH-alkoxyaryl, NHC(O)NH-alkylaminoaryl, NHC(O)NH-cycloalkyl, NHC(O)NH-aryl, NHC(O)NH-heteroaryl, NHC(O)NH-heterocycloalkyl, NHC(O)NH-alkylheterocycloalkyl, S-alkyl, S-alkenyl, S-alkynyl, S-alkoxyalkyl, S-alkylaminoalkyl, S-alkylaryl, S-alkylaminocarbonyl, S-alkylaryl, S-alkynylaryl, S-alkoxyaryl, S-alkylaminoaryl, S-cycloalkyl, S-aryl, S-heteroaryl, S-heterocycloalkyl, S-alkylheterocycloalkyl, S(O)alkyl, S(O)alkenyl, S(O)alkynyl, S(O)alkoxyalkyl, S(O)alkylaminoalkyl, S(O)alkylaminocarbonyl, S(O)alkylaryl, S(O)alkenylaryl, S(O)alkynylaryl, S(O)alkoxyaryl, S(O)alkylaminoaryl, S(O)cycloalkyl, S(O)aryl, S(O)heteroaryl, S(O)heterocycloalkyl, S(O)alkylheterocycloalkyl, S(O)₂alkyl, S(O)₂alkenyl, S(O)₂alkynyl, S(O)₂alkoxyalkyl, S(O)₂alkylaminoalkyl, S(O)₂alkylaminocarbonyl, S(O)₂alkylaryl, S(O)₂alkenylaryl, S(O)₂alkynylaryl, S(O)₂alkoxyaryl, S(O)₂alkylaminoaryl, S(O)₂cycloalkyl, S(O)₂aryl, S(O)₂heteroaryl, S(O)₂heterocycloalkyl, S(O)₂alkylheterocycloalkyl, SO₂NH, SO₂NH-alkyl, SO₂NH-alkenyl, SO₂NH-alkynyl, SO₂NH-alkylaryl, SO₂NH-alkenylaryl, SO₂NH-alkynylaryl, SO₂NH-cycloalkyl, SO₂NH-aryl, SO₂NH-heteroaryl, SO₂NH-heterocycloalkyl, SO₂NH-alkylheterocycloalkyl, alkylaryloxyalkoxy, alkylaryloxyalkylamino, alkylarylaminoalkoxy, alkylarylaminoalkylamino, alkylarylalkylaminoalkoxy, alkylarylalkylaminoalkoxy, alkenylaryloxyalkoxy, alkenylaryloxyalkylamino, alkenylarylaminoalkoxy, alkenylarylaminoalkylamino, alkenylarylalkylaminoalkoxy, alkenylarylalkylaminoalkylamino. It is understood that the alkyl, alkenyl, alkynyl, aryl, heteroaryl, cycloalkyl, heterocyclic and the like can be further substituted.

In a more preferred embodiment, B is a straight chain alkyl, alkenyl, alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, or alkynylheterocyclylalkynyl. One or more methylenes can be interrupted or terminated by —O—, —N(R₈)—, —C(O)—, —C(O)N(R₈)—, or —C(O)O—.

In the most preferred embodiment, B is selected from straight chain C1-C10 alkyl, C1-C10 alkenyl, C1-C10 alkynyl, C1-C10 alkoxy, alkoxyC1-C10alkoxy, C1-C10 alkylamino, alkoxyC1-C10alkylamino, C1-C10 alkylcarbonylamino, C1-C10 alkylaminocarbonyl, aryloxyC1-C10alkoxy, aryloxyC1-C10alkylamino, aryloxyC1-C10alkylamino carbonyl, C1-C10-alkylaminoalkylaminocarbonyl, C1-C10 alkyl(N-alkyl)aminoalkyl-aminocarbonyl, alkylaminoalkylamino, alkylcarbonylaminoalkylamino, alkyl(N-alkyl)aminoalkylamino, (N-alkyl)alkylcarbonylaminoalkylamino, alkylaminoalkyl, alkylaminoalkylaminoalkyl, alkylpiperazinoalkyl, piperazinoalkyl, alkylpiperazino, alkenylaryloxyC1-C10alkoxy, alkenylarylaminoC1-C10alkoxy, alkenylaryllalkylaminoC1-C10alkoxy, alkenylaryloxyC1-C10alkylamino, alkenylaryloxyC1-C10alkylaminocarbonyl and piperazinoalkylaryl.

In one embodiment, the multi-functional compounds of the present invention are compounds represented by formula (II) or its geometric isomers, enantiomers, diastereomers, racemates, pharmaceutically acceptable salts, prodrugs and solvates thereof:

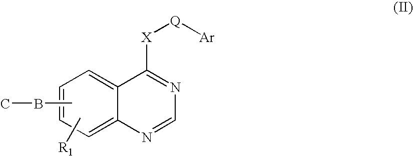

wherein
Ar is aryl, substituted aryl, heteroaryl, or substituted heteroaryl;
Q is absent or substituted or unsubstituted alkyl;
X is O, S, NH, or alkylamino;
B, C and R₁ are as previously defined.

In a most preferred embodiment, Ar is phenyl, substituted phenyl, naphthyl, substituted naphthyl, pyridinyl, substituted pyridinyl, furanyl, substituted furanyl, pyrrolyl, substituted pyrrolyl; pyrazolyl, substituted pyrazolyl, oxazolyl, substituted oxazolyl, thiophenyl, or substituted thiophenyl; Q is absent or substituted or unsubstituted alkyl; X is O, S, NH, or alkylamino; R₁ is hydrogen, hydroxy, halogen, lower alkyl, lower alkoxy, alkoxyalkoxy (preferably methoxyethoxy), alkylaminoalkoxy (preferably methylaminoethoxy), lower alkylamino or lower dialkylamino; B and C and are as previously defined in the most preferred embodiment.

In one embodiment, the multi-functional compounds of the present invention are compounds represented by formula (III) as illustrated below, or its geometric isomers, enantiomers, diastereomers, racemates, pharmaceutically acceptable salts, prodrugs and solvates thereof:

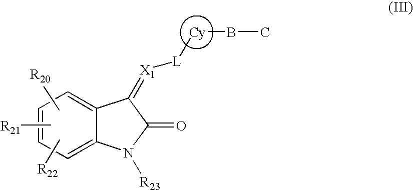

wherein
X₁ is N, CR₈; where R₈ is as previously defined;
L is absent or NH;
Cy is aryl, substituted aryl, heteroaryl, or substituted heteroaryl;
R₂₀, R₂₁, R₂₂ are independently selected from hydrogen, hydroxy, CF₃, NO₂, halogen, lower alkyl, lower alkoxy, lower alkylamino, alkoxyalkoxy (preferably methoxyethoxy), alkylaminoalkoxy (preferably methylaminoethoxy), phenyl, thiophenyl, furanyl, pyrazinyl, substituted pyrazinyl, and morpholino; and R₁₂ is selected from hydrogen or lower alkyl;
R₂₃ is hydrogen or aliphatic;
B, C, R₁, R₂, and R₃ are as previously defined.

In the most preferred embodiment, X₁ is CH, C(lower alkyl); L is absent; Cy is phenyl, substituted phenyl, pyridinyl, substituted pyridinyl, furanyl, substituted furanyl, pyrrolyl, substituted pyrrolyl; pyrazolyl, substituted pyrazolyl, oxazolyl, substituted oxazolyl, thiophenyl, or substituted thiophenyl; G is O; R₁, R₂, and R₃ are independently selected from H, OH, CF₃, NO₂, halogen, lower alkyl, lower alkoxy, alkoxyalkoxy (preferably methoxyethoxy), alkylaminoalkoxy (preferably methoxyaminoethoxy), lower alkylamino and lower dialkylamino; B and C are as previously defined in the most preferred embodiment.

In one embodiment, the multi-functional compounds of the present invention are compounds represented by formulae (IV) and (V) as illustrated below, or its geometric isomers, enantiomers, diastereomers, racemates, pharmaceutically acceptable salts, prodrugs and solvates thereof:

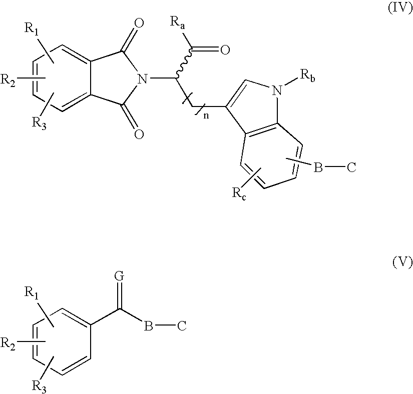

wherein
Rₐ is hydroxy, amino, alkoxy, alkylamino, dialkylamino;
R_{b} is hydrogen, aliphatic group, acyl;
R_{c} is selected from R₁;
n is 0, 1, 2, or 3;
G is S or O;
B, C and R₁, R₂, and R₃ are as previously defined.

In the most preferred embodiment, Rₐ is hydroxy, amino, alkoxy, alkylamino, dialkylamino; R_{b} is hydrogen, lower alkyl, acyl; G is O; R₁, R₂, R₃ and R_{c} are independently selected from H, OH, CF₃, NO₂, halogen, lower alkyl, lower alkoxy, alkoxyalkoxy (preferably methoxyethoxy), alkylaminoalkoxy (preferably methoxyaminoethoxy), lower alkylamino and lower dialkylamino; B and C are as previously defined in the most preferred embodiment.

In one embodiment, the multi-functional compounds of the present invention are compounds represented by formulae (VI) and (VII) as illustrated below, or its geometric isomers, enantiomers, diastereomers, racemates, pharmaceutically acceptable salts, prodrugs and solvates thereof:

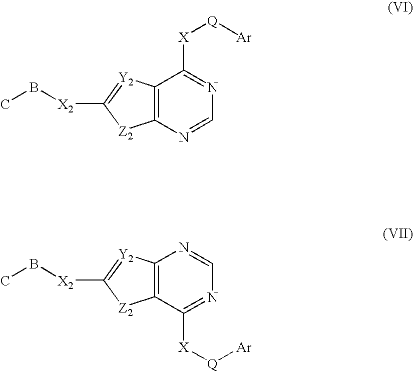

wherein
Z₂ is O, S, or NH
Y₂ is N or CR₂₀; where R₂₀ is selected from hydrogen, halogen, aliphatic, aryl, substituted aryl, heteroaryl, substituted heteroaryl;
X₂ is absent, aryl, substituted aryl, heteroaryl, substituted heteroaryl; heterocyclic; substituted heterocyclic;
B, C, Q, X and Ar are as previously defined.

In the most preferred embodiment, Z₂ is O, S, or NH; Y₂ is NH, CH, C(lower alkyl); X₂ is phenyl, substituted phenyl, pyridinyl, substituted pyridinyl, furanyl, substituted furanyl, pyrrolyl, substituted pyrrolyl; pyrazolyl, substituted pyrazolyl, oxazolyl, substituted oxazolyl, thiophenyl, or substituted thiophenyl; Ar is phenyl, substituted phenyl, naphthyl, substituted naphthyl, pyridinyl, substituted pyridinyl, furanyl, substituted furanyl, pyrrolyl, substituted pyrrolyl; pyrazolyl, substituted pyrazolyl, oxazolyl, substituted oxazolyl, thiophenyl, or substituted thiophenyl; Q is absent or substituted or unsubstituted alkyl; X is O, S, NH, or alkylamino; B and C are as previously defined in the most preferred embodiment.

In one embodiment, the multi-functional compounds of the present invention are compounds represented by formula (VIII) as illustrated below, or its geometric isomers, enantiomers, diastereomers, racemates, pharmaceutically acceptable salts, prodrugs and solvates thereof:

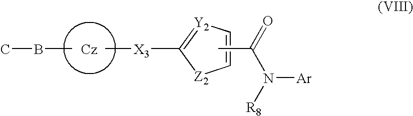

wherein
Cz is selected from aryl, substituted aryl, heteroaryl, substituted heteroaryl, and heterocylic;
X₃ is NH, O or S;
C, B, Y₂, Z₂, Ar and R₈ are as previously defined.

In the most preferred embodiment, Cz is phenyl, substituted phenyl, pyridinyl, pyrimidinyl, substituted pyrimidinyl, pyrazinyl, substituted pyrazinyl, pyrrolyl, substituted pyrrolyl, oxazolyl, substituted oxazolyl, thiazolyl, substituted thiazolyl; Y₂ is NH, CH, C(lower alkyl); Z₂ is O, S, or NH; X₃ is NH, O or S; Ar is phenyl, substituted phenyl, naphthyl, substituted naphthyl, pyridinyl, substituted pyridinyl, furanyl, substituted furanyl, pyrrolyl, substituted pyrrolyl; pyrazolyl, substituted pyrazolyl, oxazolyl, substituted oxazolyl, thiophenyl, or substituted thiophenyl; R₈ is hydrogen or lower alkyl; B and C are as previously defined in the most preferred embodiment.

In one embodiment, the multi-functional compounds of the present invention are compounds represented by formula (IX) or (X) as illustrated below, or its geometric isomers, enantiomers, diastereomers, racemates, pharmaceutically acceptable salts, prodrugs and solvates thereof:

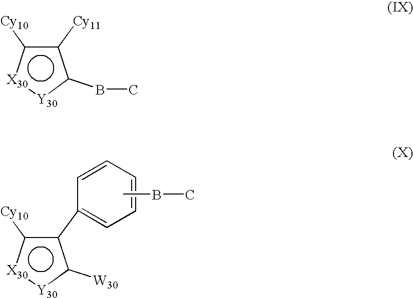

wherein
Cy₁₀ and Cy₁₁ are each independently selected from aryl, substituted aryl, heteroaryl, substituted heteroaryl, heterocyclic, substituted heterocyclic, cycloalkyl and substituted cycloalkyl;
Y₃₀ is N, NR₈ or CR₈, where R₈ is hydrogen, acyl, aliphatic or substituted aliphatic;
X₃₀ is CR₈, NR₈, N, O or S;
W₃₀ is hydrogen, acyl, aliphatic or substituted aliphatic;
B is linker;
C is as previously defined in the first embodiment.

In one embodiment, the multi-functional compounds of the present invention are compounds represented by formula (XI) as illustrated below, or its geometric isomers, enantiomers, diastereomers, racemates, pharmaceutically acceptable salts, prodrugs and solvates thereof:

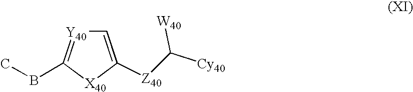

wherein
Cy₄₀ is each independently selected from aryl, substituted aryl, heteroaryl, substituted heteroaryl, heterocyclic, substituted heterocyclic, cycloalkyl and substituted cycloalkyl;
W₄₀ is each independently selected from hydrogen, halogen, acyl, aliphatic, substituted aliphatic, aryl, substituted aryl, heteroaryl, substituted heteroaryl, heterocyclic, substituted heterocyclic, cycloalkyl and substituted cycloalkyl;
Z₄₀ is O, S, S(O), SO₂, SO₂NH, NR₈, C(O) or C(O)NH₂;
Y₄₀ is N or CR₈, where R₈ is hydrogen, acyl, aliphatic or substituted aliphatic;
X₄₀ is CR₈, NR₈, O or S;
B is linker;
C is as previously defined in the first embodiment.

In one embodiment, the multi-functional compounds of the present invention are compounds represented by formula (XII) or (XIII) as illustrated below, or its geometric isomers, enantiomers, diastereomers, racemates, pharmaceutically acceptable salts, prodrugs and solvates thereof:

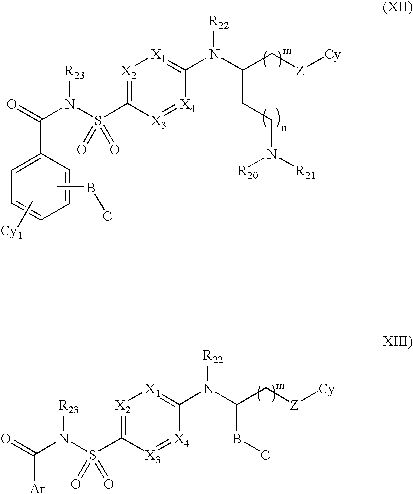

wherein
Cy and Cy₁ are each independently selected from aryl, substituted aryl, heteroaryl, substituted heteroaryl, heterocyclic, substituted heterocyclic, cycloalkyl and substituted cycloalkyl;
Ar is aryl, substituted aryl, heteroaryl, or substituted heteroaryl;
Y is N, NR₈ or CR₈, where R₈ is hydrogen, acyl, aliphatic or substituted aliphatic;
Z is O, S, CR₈, or NR₈;
R₂₀ and R₂₁ are each independently selected from hydrogen, acyl, aliphatic and substituted aliphatic; alternatively, R₂₀ and R₂₁ can be taken together with the atom they are attached to form a heterocyclic or substituted heterocyclic;
m is 1, 2 or 3;
n is 1, 2, 3 or 4;
R₂₂ and R₂₃ are each independently selected from hydrogen, acyl, aliphatic and substituted aliphatic;
X₁-X₄ are independently N or CR₂₅, where R₂₅ is independently selected from hydrogen, hydroxy, amino, halogen, substituted or unsubstituted alkoxy, substituted or unsubstituted alkylamino, substituted or unsubstituted dialkylamino, CF₃, CN, NO₂, N₃, sulfonyl, acyl, aliphatic, and substituted aliphatic;
B is linker;
C is as previously defined in the first embodiment.

In one embodiment, the multi-functional compounds of the present invention are compounds represented by formula (XIV) as illustrated below, or its geometric isomers, enantiomers, diastereomers, racemates, pharmaceutically acceptable salts, prodrugs and solvates thereof:

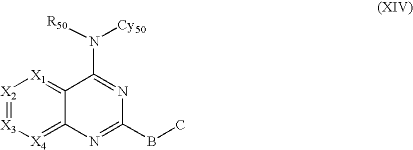

wherein
Cy₅₀ is selected from the group consisting of aryl, substituted aryl, heteroaryl, substituted heteroaryl, heterocyclic, substituted heterocyclic, cycloakyl and substituted cycloalkyl;
R₅₀ is lower alkyl;
X₁-X₄ are independently N or CR₂₁, where R₂₁ is independently selected from the group consisting of hydrogen, hydroxy, amino, halogen, lower alkoxy, lower alkylamino, CF₃, CN, NO₂, N₃, sulfonyl, acyl, C₁-C₃ alkyl, C₂-C₃ alkenyl, C₂-C₃ alkynyl;
B is linker;
C is as previously defined in the first embodiment.

In one embodiment, the multi-functional compounds of the present invention are compounds represented by formula (XV) as illustrated below, or its geometric isomers, enantiomers, diastereomers, racemates, pharmaceutically acceptable salts, prodrugs and solvates thereof:

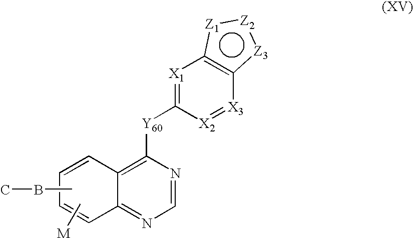

wherein
Z₁, Z₂ and Z₃ are independently selected from the group consisting of CR₂₁, NR₈, N, O or S, where R₈ is hydrogen, acyl, aliphatic or substituted aliphatic; R₂₁ is independently selected from the group consisting of hydrogen, hydroxy, amino, halogen, substituted or unsubstituted alkoxy, substituted or unsubstituted alkylamino, substituted or unsubstituted dialkylamino, CF₃, CN, NO₂, N₃, sulfonyl, acyl, aliphatic, and substituted aliphatic;
X₁-X₃ are independently C, N or CR₂₁;
Y₆₀ is NR₈, O, S, SO, SO₂, aliphatic, and substituted aliphatic;
M is independently selected from hydrogen, hydroxy, amino, halogen, CF₃, CN, N₃, NO₂, sulfonyl, acyl, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, or alkynylheterocyclylalkynyl, which one or more methylenes can be interrupted or terminated by O, S, S(O), SO₂, N(R₈), C(O), substituted or unsubstituted aryl, substituted or unsubstituted heteroaryl, substituted or unsubstituted heterocyclic; where R₈ hydrogen, acyl, aliphatic or substituted aliphatic;
B is linker;
C is as previously defined in the first embodiment.

In one embodiment, the multi-functional compounds of the present invention are compounds represented by formula (XVI) as illustrated below, or its geometric isomers, enantiomers, diastereomers, racemates, pharmaceutically acceptable salts, prodrugs and solvates thereof:

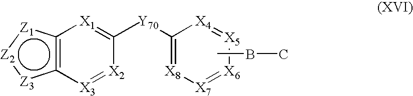

wherein
Z₁, Z₂ and Z₃ are independently selected from the group consisting of CR₂₁, NR₈, N, O or S, where R₈ is hydrogen, acyl, aliphatic or substituted aliphatic; R₂₁ is independently selected from the group consisting of hydrogen, hydroxy, amino, halogen, substituted or unsubstituted alkoxy, substituted or unsubstituted alkylamino, substituted or unsubstituted dialkylamino, CF₃, CN, NO₂, N₃, sulfonyl, acyl, aliphatic, and substituted aliphatic;
X₁-X₈ are independently C, N or CR₂₁;
Y₇₀ is NR₈, O, S, SO, SO₂, aliphatic, and substituted aliphatic;
B is linker;
C is as previously defined in the first embodiment.

In one embodiment, the multi-functional compounds of the present invention are compounds represented by formula (XVII) as illustrated below, or its geometric isomers, enantiomers, diastereomers, racemates, pharmaceutically acceptable salts, prodrugs and solvates thereof:

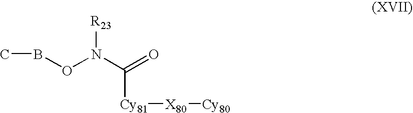

wherein
Cy₈₀ and Cy₈₁ are each independently selected from aryl, substituted aryl, heteroaryl, substituted heteroaryl, heterocyclic, substituted heterocyclic, cycloalkyl and substituted cycloalkyl;
X₈₀ is NR₈, O, S, SO, SO₂, CO alkyl or substituted alkyl;
R₂₃ is hydrogen, aliphatic, substituted aliphatic or acyl;
B is linker;
C is as previously defined in the first embodiment.

The invention further provides methods for the prevention or treatment of diseases or conditions involving aberrant proliferation, differentiation or survival of cells. In one embodiment, the invention further provides for the use of one or more compounds of the invention in the manufacture of a medicament for halting or decreasing diseases involving aberrant proliferation, differentiation, or survival of cells. In preferred embodiments, the disease is cancer. In one embodiment, the invention relates to a method of treating cancer in a subject in need of treatment comprising administering to said subject a therapeutically effective amount of a compound of the invention.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non small cell), breast cancer, pancreatic cancer, melanoma and other skin cancers, stomach cancer, brain tumors, tumors related to Gorlin's syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familiary adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the invention, the present invention provides for the use of one or more compounds of the invention in the manufacture of a medicament for the treatment of cancer.

In one embodiment, the present invention includes the use of one or more compounds of the invention in the manufacture of a medicament that prevents further aberrant proliferation, differentiation, or survival of cells. For example, compounds of the invention may be useful in preventing tumors from increasing in size or from reaching a metastatic state. The subject compounds may be administered to halt the progression or advancement of cancer or to induce tumor apoptosis or to inhibit tumor angiogenesis. In addition, the instant invention includes use of the subject compounds to prevent a recurrence of cancer.

This invention further embraces the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias or pre-cancerous lesions from continuing to expand or from becoming cancerous. Examples of pre-cancerous lesions may occur in skin, esophageal tissue, breast and cervical intra-epithelial tissue.

“Combination therapy” includes the administration of the subject compounds in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, preferably compounds that are able to enhance the effect of the compounds of the invention. The compounds of the invention can be administered simultaneously (as a single preparation or separate preparation) or sequentially to the other drug therapy. In general, a combination therapy envisions administration of two or more drugs during a single cycle or course of therapy.

“Combination therapy” includes the administration of the subject compounds in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, preferably compounds that are able to enhance the effect of the compounds of the invention. The compounds of the invention can be administered simultaneously (as a single preparation or separate preparation) or sequentially to the other drug therapy. In general, a combination therapy envisions administration of two or more drugs during a single cycle or course of therapy.

In one aspect of the invention, the subject compounds may be administered in combination with one or more separate agents that modulate protein kinases involved in various disease states. Examples of such kinases may include, but are not limited to: serine/threonine specific kinases, receptor tyrosine specific kinases and non-receptor tyrosine specific kinases. Serine/threonine kinases include mitogen activated protein kinases (MAPK), meiosis specific kinase (MEK), RAF and aurora kinase. Examples of receptor kinase families include epidermal growth factor receptor (EGFR) (e.g. HER2/neu, HER3, HER4, ErbB, ErbB2, ErbB3, ErbB4, Xmrk, DER, Let23); fibroblast growth factor (FGF) receptor (e.g. FGF-R1, GFF-R2/BEK/CEK3, FGF-R3/CEK2, FGF-R4/TKF, KGF-R); hepatocyte growth/scatter factor receptor (HGFR) (e.g, MET, RON, SEA, SEX); insulin receptor (e.g. IGFI-R); Eph (e.g. CEK5, CEK8, EBK, ECK, EEK, EHK-1, EHK-2, ELK, EPH, ERK, HEK, MDK2, MDK5, SEK); Axl (e.g. Mer/Nyk, Rse); RET; and platelet-derived growth factor receptor (PDGFR) (e.g. PDGFα-R, PDGβ-R, CSF1-R/FMS, SCF-R/C-KIT, VEGF-R/FLT, NEK/FLK1, FLT3/FLK2/STK-1). Non-receptor tyrosine kinase families include, but are not limited to, BCR-ABL (e.g. p43^{abl}, ARG); BTK (e.g. ITK/EMT, TEC); CSK, FAK, FPS, JAK, SRC, BMX, FER, CDK and SYK.

In another aspect of the invention, the subject compounds may be administered in combination with one or more separate agents that modulate non-kinase biological targets or processes. Such targets include histone deacetylases (HDAC), DNA methyltransferase (DNMT), heat shock proteins (e.g. HSP90), and proteosomes.

In a preferred embodiment, subject compounds may be combined with antineoplastic agents (e.g. small molecules, monoclonal antibodies, antisense RNA, and fusion proteins) that inhibit one or more biological targets such as Zolinza, Tarceva, Iressa, Tykerb, Gleevec, Sutent, Sprycel, Nexavar, Sorafinib, CNF2024, RG108, BMS387032, Affinitak, Avastin, Herceptin, Erbitux, AG24322, PD325901, ZD6474, PD184322, Obatodax, ABT737 and AEE788. Such combinations may enhance therapeutic efficacy over efficacy achieved by any of the agents alone and may prevent or delay the appearance of resistant mutational variants.

In certain preferred embodiments, the compounds of the invention are administered in combination with a chemotherapeutic agent. Chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as mustard gas derivatives (Mechlorethamine, cylophosphamide, chlorambucil, melphalan, ifosfamide), ethylenimines (thiotepa, hexamethylmelanine), Alkylsulfonates (Busulfan), Hydrazines and Triazines (Altretamine, Procarbazine, Dacarbazine and Temozolomide), Nitrosoureas (Carmustine, Lomustine and Streptozocin), Ifosfamide and metal salts (Carboplatin, Cisplatin, and Oxaliplatin); plant alkaloids such as Podophyllotoxins (Etoposide and Tenisopide), Taxanes (Paclitaxel and Docetaxel), Vinca alkaloids (Vincristine, Vinblastine, Vindesine and Vinorelbine), and Camptothecan analogs (Irinotecan and Topotecan); anti-tumor antibiotics such as Chromomycins (Dactinomycin and Plicamycin), Anthracyclines (Doxorubicin, Daunorubicin, Epirubicin, Mitoxantrone, Valrubicin and Idarubicin), and miscellaneous antibiotics such as Mitomycin, Actinomycin and Bleomycin; anti-metabolites such as folic acid antagonists (Methotrexate, Pemetrexed, Raltitrexed, Aminopterin), pyrimidine antagonists (5-Fluorouracil, Floxuridine, Cytarabine, Capecitabine, and Gemcitabine), purine antagonists (6-Mercaptopurine and 6-Thioguanine) and adenosine deaminase inhibitors (Cladribine, Fludarabine, Mercaptopurine, Clofarabine, Thioguanine, Nelarabine and Pentostatin); topoisomerase inhibitors such as topoisomerase I inhibitors (Ironotecan, topotecan) and topoisomerase II inhibitors (Amsacrine, etoposide, etoposide phosphate, teniposide); monoclonal antibodies (Alemtuzumab, Gemtuzumab ozogamicin, Rituximab, Trastuzumab, Ibritumomab Tioxetan, Cetuximab, Panitumumab, Tositumomab, Bevacizumab); and miscellaneous anti-neoplastics such as ribonucleotide reductase inhibitors (Hydroxyurea); adrenocortical steroid inhibitor (Mitotane); enzymes (Asparaginase and Pegaspargase); anti-microtubule agents (Estramustine); and retinoids (Bexarotene, Isotretinoin, Tretinoin (ATRA)).

In certain preferred embodiments, the compounds of the invention are administered in combination with a chemoprotective agent. Chemoprotective agents act to protect the body or minimize the side effects of chemotherapy. Examples of such agents include, but are not limited to, amfostine, mesna, and dexrazoxane.

In one aspect of the invention, the subject compounds are administered in combination with radiation therapy. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

It will be appreciated that compounds of the invention can be used in combination with an immunotherapeutic agent. One form of immunotherapy is the generation of an active systemic tumor-specific immune response of host origin by administering a vaccine composition at a site distant from the tumor. Various types of vaccines have been proposed, including isolated tumor-antigen vaccines and anti-idiotype vaccines. Another approach is to use tumor cells from the subject to be treated, or a derivative of such cells (reviewed by Schirrmacher et al. (1995) J. Cancer Res. Clin. Oncol. 121:487). In U.S. Pat. No. 5,484,596, Hanna Jr. et al claims a method for treating a resectable carcinoma to prevent recurrence or metastases, comprising surgically removing the tumor, dispersing the cells with collagenase, irradiating the cells, and vaccinating the patient with at least three consecutive doses of about 10⁷ cells.

It will be appreciated that the compounds of the invention may advantageously be used in conjunction with one or more adjunctive therapeutic agents. Examples of suitable agents for adjunctive therapy include a 5HT₁ agonist, such as a triptan (e.g. sumatriptan or naratriptan); an adenosine A1 agonist; an EP ligand; an NMDA modulator, such as a glycine antagonist; a sodium channel blocker (e.g. lamotrigine); a substance P antagonist (e.g. an NK₁ antagonist); a cannabinoid; acetaminophen or phenacetin; a 5-lipoxygenase inhibitor; a leukotriene receptor antagonist; a DMARD (e.g. methotrexate); gabapentin and related compounds; a tricyclic antidepressant (e.g. amitryptilline); a neurone stabilising antiepileptic drug; a mono-aminergic uptake inhibitor (e.g. venlafaxine); a matrix metalloproteinase inhibitor; a nitric oxide synthase (NOS) inhibitor, such as an iNOS or an nNOS inhibitor; an inhibitor of the release, or action, of tumour necrosis factor .alpha.; an antibody therapy, such as a monoclonal antibody therapy; an antiviral agent, such as a nucleoside inhibitor (e.g. lamivudine) or an immune system modulator (e.g. interferon); an opioid analgesic; a local anaesthetic; a stimulant, including caffeine; an H₂-antagonist (e.g. ranitidine); a proton pump inhibitor (e.g. omeprazole); an antacid (e.g. aluminium or magnesium hydroxide; an antiflatulent (e.g. simethicone); a decongestant (e.g. phenylephrine, phenylpropanolamine, pseudoephedrine, oxymetazoline, epinephrine, naphazoline, xylometazoline, propylhexedrine, or levo-desoxyephedrine); an antitussive (e.g. codeine, hydrocodone, carmiphen, carbetapentane, or dextramethorphan); a diuretic; or a sedating or non-sedating antihistamine.

Matrix metalloproteinases (MMPs) are a family of zinc-dependent neutral endopeptidases collectively capable of degrading essentially all matrix components. Over 20 MMP modulating agents are in pharmaceutical develop, almost half of which are indicated for cancer. The University of Toronto researchers have reported that HDACs regulate MMP expression and activity in 3T3 cells. In particular, inhibition of HDAC by trichostatin A (TSA), which has been shown to prevent tumorigenesis and metastasis, decreases mRNA as well as zymographic activity of gelatinase A (MMP2; Type IV collagenase), a matrix metalloproteinase, which is itself, implicated in tumorigenesis and metastasis (Ailenberg M., Silverman M., Biochem Biophys Res Commun. 2002, 298:110-115). Another recent article that discusses the relationship of HDAC and MMPs can be found in Young D. A., et al., Arthritis Research & Therapy, 2005, 7: 503. Furthermore, the commonality between HDAC and MMPs inhibitors is their zinc-binding functionality. Therefore, in one aspect of the invention, compounds of the invention can be used as MMP inhibitors and may be of use in the treatment of disorders relating to or associated with dysregulation of MMP. The overexpression and activation of MMPs are known to induce tissue destruction and are also associated with a number of specific diseases including rheumatoid arthritis, periodontal disease, cancer and atherosclerosis.

The compounds may also be used in the treatment of a disorder involving, relating to or, associated with dysregulation of histone deacetylase (HDAC). There are a number of disorders that have been implicated by or known to be mediated at least in part by HDAC activity, where HDAC activity is known to play a role in triggering disease onset, or whose symptoms are known or have been shown to be alleviated by HDAC inhibitors. Disorders of this type that would be expected to be amenable to treatment with the compounds of the invention include the following but not limited to: Anti-proliferative disorders (e.g. cancers); Neurodegenerative diseases including Huntington's disease, Polyglutamine disease, Parkinson's disease, Alzheimer's disease, Seizures, Striatonigral degeneration, Progressive supranuclear palsy, Torsion dystonia, Spasmodic torticollis and dyskinesis, Familial tremor, Gilles de la Tourette syndrome, Diffuse Lewy body disease, Progressive supranuclear palsy, Pick's disease, intracerebral hemorrhage, Primary lateral sclerosis, Spinal muscular atrophy, Amyotrophic lateral sclerosis, Hypertrophic interstitial polyneuropathy, Retinitis pigmentosa, Hereditary optic atrophy, Hereditary spastic paraplegia, Progressive ataxia and Shy-Drager syndrome; Metabolic diseases including Type 2 diabetes; Degenerative diseases of the Eye including Glaucoma, Age-related macular degeneration, Rubeotic glaucoma; Inflammatory diseases and/or Immune system disorders including Rheumatoid Arthritis (RA), Osteoarthritis, Juvenile chronic arthritis, Graft versus Host disease, Psoriasis, Asthma, Spondyloarthropathy, Crohn's Disease, inflammatory bowel disease Colitis Ulcerosa, Alcoholic hepatitis, Diabetes, Sjoegrens's syndrome, Multiple Sclerosis, Ankylosing spondylitis, Membranous glomerulopathy, Discogenic pain, Systemic Lupus Erythematosus; Disease involving angiogenesis including cancer, psoriasis, rheumatoid arthritis; Psychological disorders including bipolar disease, schizophrenia, mania, depression and dementia; Cardiovascular Diseases including heart failure, restenosis and arteriosclerosis; Fibrotic diseases including liver fibrosis, cystic fibrosis and angiofibroma; Infectious diseases including Fungal infections, such as Candida Albicans, Bacterial infections, Viral infections, such as Herpes Simplex, Protozoal infections, such as Malaria, Leishmania infection, Trypanosoma brucei infection, Toxoplasmosis and coccidlosis and Haematopoietic disorders including thalassemia, anemia and sickle cell anemia.

In one embodiment, compounds of the invention can be used to induce or inhibit apoptosis, a physiological cell death process critical for normal development and homeostasis. Alterations of apoptotic pathways contribute to the pathogenesis of a variety of human diseases. Compounds of the invention, as modulators of apoptosis, will be useful in the treatment of a variety of human diseases with aberrations in apoptosis including cancer (particularly, but not limited to, follicular lymphomas, carcinomas with p53 mutations, hormone dependent tumors of the breast, prostate and ovary, and precancerous lesions such as familial adenomatous polyposis), viral infections (including, but not limited to, herpes virus, poxvirus, Epstein-Barr virus, Sindbis virus and adenovirus), autoimmune diseases (including, but not limited to, systemic lupus, erythematosus, immune mediated glomerulonephritis, rheumatoid arthritis, psoriasis, inflammatory bowel diseases, and autoimmune diabetes mellitus), neurodegenerative disorders (including, but not limited to, Alzheimer's disease, AIDS-related dementia, Parkinson's disease, amyotrophic lateral sclerosis, retinitis pigmentosa, spinal muscular atrophy and cerebellar degeneration), AIDS, myelodysplastic syndromes, aplastic anemia, ischemic injury associated myocardial infarctions, stroke and reperfusion injury, arrhythmia, atherosclerosis, toxin-induced or alcohol induced liver diseases, hematological diseases (including, but not limited to, chronic anemia and aplastic anemia), degenerative diseases of the musculoskeletal system (including, but not limited to, osteoporosis and arthritis), aspirin-sensitive rhinosinusitis, cystic fibrosis, multiple sclerosis, kidney diseases, and cancer pain.

In one aspect, the invention provides the use of compounds of the invention for the treatment and/or prevention of immune response or immune-mediated responses and diseases, such as the prevention or treatment of rejection following transplantation of synthetic or organic grafting materials, cells, organs or tissue to replace all or part of the function of tissues, such as heart, kidney, liver, bone marrow, skin, cornea, vessels, lung, pancreas, intestine, limb, muscle, nerve tissue, duodenum, small-bowel, pancreatic-islet-cell, including xeno-transplants, etc.; to treat or prevent graft-versus-host disease, autoimmune diseases, such as rheumatoid arthritis, systemic lupus erythematosus, thyroiditis, Hashimoto's thyroiditis, multiple sclerosis, myasthenia gravis, type I diabetes uveitis, juvenile-onset or recent-onset diabetes mellitus, uveitis, Graves disease, psoriasis, atopic dermatitis, Crohn's disease, ulcerative colitis, vasculitis, auto-antibody mediated diseases, aplastic anemia, Evan's syndrome, autoimmune hemolytic anemia, and the like; and further to treat infectious diseases causing aberrant immune response and/or activation, such as traumatic or pathogen induced immune disregulation, including for example, that which are caused by hepatitis B and C infections, HIV, staphylococcus aureus infection, viral encephalitis, sepsis, parasitic diseases wherein damage is induced by an inflammatory response (e.g., leprosy); and to prevent or treat circulatory diseases, such as arteriosclerosis, atherosclerosis, vasculitis, polyarteritis nodosa and myocarditis. In addition, the present invention may be used to prevent/suppress an immune response associated with a gene therapy treatment, such as the introduction of foreign genes into autologous cells and expression of the encoded product. Thus in one embodiment, the invention relates to a method of treating an immune response disease or disorder or an immune-mediated response or disorder in a subject in need of treatment comprising administering to said subject a therapeutically effective amount of a compound of the invention.

In one aspect, the invention provides the use of compounds of the invention in the treatment of a variety of neurodegenerative diseases, a non-exhaustive list of which includes: I. Disorders characterized by progressive dementia in the absence of other prominent neurologic signs, such as Alzheimer's disease; Senile dementia of the Alzheimer type; and Pick's disease (lobar atrophy); II. Syndromes combining progressive dementia with other prominent neurologic abnormalities such as A) syndromes appearing mainly in adults (e.g., Huntington's disease, Multiple system atrophy combining dementia with ataxia and/or manifestations of Parkinson's disease, Progressive supranuclear palsy (Steel-Richardson-Olszewski), diffuse Lewy body disease, and corticodentatonigral degeneration); and B) syndromes appearing mainly in children or young adults (e.g., Hallervorden-Spatz disease and progressive familial myoclonic epilepsy); III. Syndromes of gradually developing abnormalities of posture and movement such as paralysis agitans (Parkinson's disease), striatonigral degeneration, progressive supranuclear palsy, torsion dystonia (torsion spasm; dystonia musculorum deformans), spasmodic torticollis and other dyskinesis, familial tremor, and Gilles de la Tourette syndrome; IV. Syndromes of progressive ataxia such as cerebellar degenerations (e.g., cerebellar cortical degeneration and olivopontocerebellar atrophy (OPCA)); and spinocerebellar degeneration (Friedreich's atazia and related disorders); V. Syndrome of central autonomic nervous system failure (Shy-Drager syndrome); VI. Syndromes of muscular weakness and wasting without sensory changes (motorneuron disease such as amyotrophic lateral sclerosis, spinal muscular atrophy (e.g., infantile spinal muscular atrophy (Werdnig-Hoffman), juvenile spinal muscular atrophy (Wohlfart-Kugelberg-Welander) and other forms of familial spinal muscular atrophy), primary lateral sclerosis, and hereditary spastic paraplegia; VII. Syndromes combining muscular weakness and wasting with sensory changes (progressive neural muscular atrophy; chronic familial polyneuropathies) such as peroneal muscular atrophy (Charcot-Marie-Tooth), hypertrophic interstitial polyneuropathy (Dejerine-Sottas), and miscellaneous forms of chronic progressive neuropathy; VIII Syndromes of progressive visual loss such as pigmentary degeneration of the retina (retinitis pigmentosa), and hereditary optic atrophy (Leber's disease). Furthermore, compounds of the invention can be implicated in chromatin remodeling.

The invention encompasses pharmaceutical compositions comprising pharmaceutically acceptable salts of the compounds of the invention as described above. The invention also encompasses pharmaceutical compositions comprising hydrates of the compounds of the invention. The term “hydrate” includes but is not limited to hemihydrate, monohydrate, dihydrate, trihydrate and the like. The invention further encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

The compounds of the invention, and derivatives, fragments, analogs, homologs, pharmaceutically acceptable salts or hydrate thereof can be incorporated into pharmaceutical compositions suitable for administration, together with a pharmaceutically acceptable carrier or excipient. Such compositions typically comprise a therapeutically effective amount of any of the compounds above, and a pharmaceutically acceptable carrier. Preferably, the effective amount when treating cancer is an amount effective to selectively induce terminal differentiation of suitable neoplastic cells and less than an amount which causes toxicity in a patient.

Compounds of the invention may be administered by any suitable means, including, without limitation, parenteral, intravenous, intramuscular, subcutaneous, implantation, oral, sublingual, buccal, nasal, pulmonary, transdermal, topical, vaginal, rectal, and transmucosal administrations or the like. Topical administration can also involve the use of transdermal administration such as transdermal patches or iontophoresis devices. Pharmaceutical preparations include a solid, semisolid or liquid preparation (tablet, pellet, troche, capsule, suppository, cream, ointment, aerosol, powder, liquid, emulsion, suspension, syrup, injection etc.) containing a compound of the invention as an active ingredient, which is suitable for selected mode of administration. In one embodiment, the pharmaceutical compositions are administered orally, and are thus formulated in a form suitable for oral administration, i.e., as a solid or a liquid preparation. Suitable solid oral formulations include tablets, capsules, pills, granules, pellets, sachets and effervescent, powders, and the like. Suitable liquid oral formulations include solutions, suspensions, dispersions, emulsions, oils and the like. In one embodiment of the present invention, the composition is formulated in a capsule. In accordance with this embodiment, the compositions of the present invention comprise in addition to the active compound and the inert carrier or diluent, a hard gelatin capsule.

Any inert excipient that is commonly used as a carrier or diluent may be used in the formulations of the present invention, such as for example, a gum, a starch, a sugar, a cellulosic material, an acrylate, or mixtures thereof. A preferred diluent is microcrystalline cellulose. The compositions may further comprise a disintegrating agent (e.g., croscarmellose sodium) and a lubricant (e.g., magnesium stearate), and may additionally comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release formulations.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

Daily administration may be repeated continuously for a period of several days to several years. Oral treatment may continue for between one week and the life of the patient. Preferably the administration may take place for five consecutive days after which time the patient can be evaluated to determine if further administration is required. The administration can be continuous or intermittent, e.g., treatment for a number of consecutive days followed by a rest period. The compounds of the present invention may be administered intravenously on the first day of treatment, with oral administration on the second day and all consecutive days thereafter.

The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions and the like as detailed above.

The amount of the compound administered to the patient is less than an amount that would cause toxicity in the patient. In certain embodiments, the amount of the compound that is administered to the patient is less than the amount that causes a concentration of the compound in the patient's plasma to equal or exceed the toxic level of the compound. Preferably, the concentration of the compound in the patient's plasma is maintained at about 10 nM. In one embodiment, the concentration of the compound in the patient's plasma is maintained at about 25 nM. In one embodiment, the concentration of the compound in the patient's plasma is maintained at about 50 nM. In one embodiment, the concentration of the compound in the patient's plasma is maintained at about 100 nM. In one embodiment, the concentration of the compound in the patient's plasma is maintained at about 500 nM. In one embodiment, the concentration of the compound in the patient's plasma is maintained at about 1000 nM.

In one embodiment, the concentration of the compound in the patient's plasma is maintained at about 2500 nM. In one embodiment, the concentration of the compound in the patient's plasma is maintained at about 5000 nM. The optimal amount of the compound that should be administered to the patient in the practice of the present invention will depend on the particular compound used and the type of cancer being treated.

### DEFINITIONS

Listed below are definitions of various terms used to describe this invention. These definitions apply to the terms as they are used throughout this specification and claims, unless otherwise limited in specific instances, either individually or as part of a larger group.

An “aliphatic group” or “aliphatic” is non-aromatic moiety that may be saturated (e.g. single bond) or contain one or more units of unsaturation, (e.g., double and/or triple bonds). An aliphatic group may be straight chained, branched or cyclic, contain carbon, hydrogen or, optionally, one or more heteroatoms and may be substituted or unsubstituted. An aliphatic group preferably contains between about 1 and about 24 atoms, more preferably between about 4 to about 24 atoms, more preferably between about 4-12 atoms, more typically between about 4 and about 8 atoms.

The term “acyl” refers to hydrogen, alkyl, partially saturated or fully saturated cycloalkyl, partially saturated or fully saturated heterocycle, aryl, and heteroaryl substituted carbonyl groups. For example, acyl includes groups such as (C₁-C₆)alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions. When indicated as being “optionally substituted”, the acyl group may be unsubstituted or optionally substituted with one or more substituents (typically, one to three substituents) independently selected from the group of substituents listed below in the definition for “substituted” or the alkyl, cycloalkyl, heterocycle, aryl and heteroaryl portion of the acyl group may be substituted as described above in the preferred and more preferred list of substituents, respectively.

For simplicity, chemical moieties are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about eight carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about ten carbon atoms and more preferably about two to about eight carbon atoms. Examples of alkenyl radicals include ethenyl, allyl, propenyl, butenyl and 4-methylbutenyl. The terms “alkenyl”, and “lower alkenyl”, embrace radicals having “cis” and “trans” orientations, or alternatively, “E” and “Z” orientations.

The term “alkynyl” embraces linear or branched radicals having at least one carbon-carbon triple bond of two to about twenty carbon atoms or, preferably, two to about twelve carbon atoms. More preferred alkynyl radicals are “lower alkynyl” radicals having two to about ten carbon atoms and more preferably about two to about eight carbon atoms. Examples of alkynyl radicals include propargyl, 1-propynyl, 2-propynyl, 1-butyne, 2-butynyl and 1-pentynyl.

The term “cycloalkyl” embraces saturated carbocyclic radicals having three to about twelve carbon atoms. The term “cycloalkyl” embraces saturated carbocyclic radicals having three to about twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The term “cycloalkenyl” embraces partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The term “alkoxy” embraces linear or branched oxy-containing radicals each having alkyl portions of one to about twenty carbon atoms or, preferably, one to about twelve carbon atoms. More preferred alkoxy radicals are “lower alkoxy” radicals having one to about ten carbon atoms and more preferably having one to about eight carbon atoms. Examples of such radicals include methoxy, ethoxy, propoxy, butoxy and tert-butoxy.

The term “alkoxyalkyl” embraces alkyl radicals having one or more alkoxy radicals attached to the alkyl radical, that is, to form monoalkoxyalkyl and dialkoxyalkyl radicals.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl.

The term “carbonyl”, whether used alone or with other terms, such as “alkoxycarbonyl”, denotes (C═O).

The term “carbanoyl”, whether used alone or with other terms, such as “arylcarbanoylyalkyl”, denotes C(O)NH.

The terms “heterocyclyl”, “heterocycle” “heterocyclic” or “heterocyclo” embrace saturated, partially unsaturated and unsaturated heteroatom-containing ring-shaped radicals, which can also be called “heterocyclyl”, “heterocycloalkenyl” and “heteroaryl” correspondingly, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The term “heteroaryl” embraces unsaturated heterocyclyl radicals. Examples of heteroaryl radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like.

The term “heterocycloalkyl” embraces heterocyclo-substituted alkyl radicals. More preferred heterocycloalkyl radicals are “lower heterocycloalkyl” radicals having one to six carbon atoms in the heterocycloalkyl radicals.

The term “alkylthio” embraces radicals containing a linear or branched alkyl radical, of one to about ten carbon atoms attached to a divalent sulfur atom. Preferred alkylthio radicals have alkyl radicals of one to about twenty carbon atoms or, preferably, one to about twelve carbon atoms. More preferred alkylthio radicals have alkyl radicals are “lower alkylthio” radicals having one to about ten carbon atoms. Most preferred are alkylthio radicals having lower alkyl radicals of one to about eight carbon atoms. Examples of such lower alkylthio radicals are methylthio, ethylthio, propylthio, butylthio and hexylthio.

The terms “aralkyl” or “arylalkyl” embrace aryl-substituted alkyl radicals such as benzyl, diphenylmethyl, triphenylmethyl, phenylethyl, and diphenylethyl.

The term “aryloxy” embraces aryl radicals attached through an oxygen atom to other radicals.

The terms “aralkoxy” or “arylalkoxy” embrace aralkyl radicals attached through an oxygen atom to other radicals.

The term “aminoalkyl” embraces alkyl radicals substituted with amino radicals. Preferred aminoalkyl radicals have alkyl radicals having about one to about twenty carbon atoms or, preferably, one to about twelve carbon atoms. More preferred aminoalkyl radicals are “lower aminoalkyl” that have alkyl radicals having one to about ten carbon atoms. Most preferred are aminoalkyl radicals having lower alkyl radicals having one to eight carbon atoms. Examples of such radicals include aminomethyl, aminoethyl, and the like.

The term “alkylamino” denotes amino groups which are substituted with one or two alkyl radicals. Preferred alkylamino radicals have alkyl radicals having about one to about twenty carbon atoms or, preferably, one to about twelve carbon atoms. More preferred alkylamino radicals are “lower alkylamino” that have alkyl radicals having one to about ten carbon atoms. Most preferred are alkylamino radicals having lower alkyl radicals having one to about eight carbon atoms. Suitable lower alkylamino may be monosubstituted N-alkylamino or disubstituted N,N-alkylamino, such as N-methylamino, N-ethylamino, N,N-dimethylamino, N,N-diethylamino or the like.

The term “linker” means an organic moiety that connects two parts of a compound. Linkers typically comprise a direct bond or an atom such as oxygen or sulfur, a unit such as NR₈, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R₈), C(O), substituted or unsubstituted aryl, substituted or unsubstituted heteroaryl, substituted or unsubstituted heterocyclic; where R₈ is hydrogen, acyl, aliphatic or substituted aliphatic. In one embodiment, the linker B is between 1-24 atoms, preferably 4-24 atoms, preferably 4-18 atoms, more preferably 4-12 atoms, and most preferably about 4-10 atoms.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent may be further substituted.

The terms “halogen” or “halo” as used herein, refers to an atom selected from fluorine, chlorine, bromine and iodine.

As used herein, the term “aberrant proliferation” refers to abnormal cell growth.

The phrase “adjunctive therapy” encompasses treatment of a subject with agents that reduce or avoid side effects associated with the combination therapy of the present invention, including, but not limited to, those agents, for example, that reduce the toxic effect of anticancer drugs, e.g., bone resorption inhibitors, cardioprotective agents; prevent or reduce the incidence of nausea and vomiting associated with chemotherapy, radiotherapy or operation; or reduce the incidence of infection associated with the administration of myelosuppressive anticancer drugs.

The term “angiogenesis,” as used herein, refers to the formation of blood vessels. Specifically, angiogenesis is a multi-step process in which endothelial cells focally degrade and invade through their own basement membrane, migrate through interstitial stroma toward an angiogenic stimulus, proliferate proximal to the migrating tip, organize into blood vessels, and reattach to newly synthesized basement membrane (see Folkman et al., Adv. Cancer Res., Vol. 43, pp. 175-203 (1985)). Anti-angiogenic agents interfere with this process. Examples of agents that interfere with several of these steps include thrombospondin-1, angiostatin, endostatin, interferon alpha and compounds such as matrix metalloproteinase (MMP) inhibitors that block the actions of enzymes that clear and create paths for newly forming blood vessels to follow; compounds, such as .alpha.v.beta.3 inhibitors, that interfere with molecules that blood vessel cells use to bridge between a parent blood vessel and a tumor; agents, such as specific COX-2 inhibitors, that prevent the growth of cells that form new blood vessels; and protein-based compounds that simultaneously interfere with several of these targets.

The term “apoptosis” as used herein refers to programmed cell death as signaled by the nuclei in normally functioning human and animal cells when age or state of cell health and condition dictates. An “apoptosis inducing agent” triggers the process of programmed cell death.

The term “cancer” as used herein denotes a class of diseases or disorders characterized by uncontrolled division of cells and the ability of these cells to invade other tissues, either by direct growth into adjacent tissue through invasion or by implantation into distant sites by metastasis.

The term “compound” is defined herein to include pharmaceutically acceptable salts, solvates, hydrates, polymorphs, enantiomers, diastereoisomers, racemates and the like of the compounds having a formula as set forth herein.

The term “devices” refers to any appliance, usually mechanical or electrical, designed to perform a particular function.

As used herein, the term “dysplasia” refers to abnormal cell growth, and typically refers to the earliest form of pre-cancerous lesion recognizable in a biopsy by a pathologist.

The term “hyperplasia,” as used herein, refers to excessive cell division or growth.

The phrase an “immunotherapeutic agent” refers to agents used to transfer the immunity of an immune donor, e.g., another person or an animal, to a host by inoculation. The term embraces the use of serum or gamma globulin containing performed antibodies produced by another individual or an animal; nonspecific systemic stimulation; adjuvants; active specific immunotherapy; and adoptive immunotherapy. Adoptive immunotherapy refers to the treatment of a disease by therapy or agents that include host inoculation of sensitized lymphocytes, transfer factor, immune RNA, or antibodies in serum or gamma globulin.

The term “inhibition,” in the context of neoplasia, tumor growth or tumor cell growth, may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, is referred to herein as prevention or chemoprevention.

The term “metastasis,” as used herein, refers to the migration of cancer cells from the original tumor site through the blood and lymph vessels to produce cancers in other tissues. Metastasis also is the term used for a secondary cancer growing at a distant site.

The term “neoplasm,” as used herein, refers to an abnormal mass of tissue that results from excessive cell division. Neoplasms may be benign (not cancerous), or malignant (cancerous) and may also be called a tumor. The term “neoplasia” is the pathological process that results in tumor formation.

As used herein, the term “pre-cancerous” refers to a condition that is not malignant, but is likely to become malignant if left untreated.

The term “proliferation” refers to cells undergoing mitosis.

The phrase a “radio therapeutic agent” refers to the use of electromagnetic or particulate radiation in the treatment of neoplasia.

The term “recurrence” as used herein refers to the return of cancer after a period of remission. This may be due to incomplete removal of cells from the initial cancer and may occur locally (the same site of initial cancer), regionally (in vicinity of initial cancer, possibly in the lymph nodes or tissue), and/or distally as a result of metastasis.

The term “treatment” refers to any process, action, application, therapy, or the like, wherein a mammal, including a human being, is subject to medical aid with the object of improving the mammal's condition, directly or indirectly.

The term “vaccine” includes agents that induce the patient's immune system to mount an immune response against the tumor by attacking cells that express tumor associated antigens (Tas).

As used herein, the term “effective amount of the subject compounds,” with respect to the subject method of treatment, refers to an amount of the subject compound which, when delivered as part of desired dose regimen, brings about, e.g. a change in the rate of cell proliferation and/or state of differentiation and/or rate of survival of a cell to clinically acceptable standards. This amount may further relieve to some extent one or more of the symptoms of a neoplasia disorder, including, but is not limited to: 1) reduction in the number of cancer cells; 2) reduction in tumor size; 3) inhibition (i.e., slowing to some extent, preferably stopping) of cancer cell infiltration into peripheral organs; 4) inhibition (i.e., slowing to some extent, preferably stopping) of tumor metastasis; 5) inhibition, to some extent, of tumor growth; 6) relieving or reducing to some extent one or more of the symptoms associated with the disorder; and/or 7) relieving or reducing the side effects associated with the administration of anticancer agents.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid or inorganic acid. Examples of pharmaceutically acceptable nontoxic acid addition salts include, but are not limited to, salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid lactobionic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of the invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

As used herein, “pharmaceutically acceptable carrier” is intended to include any and all solvents, dispersion media, coatings, antibacterial and antifungal agents, isotonic and absorption delaying agents, and the like, compatible with pharmaceutical administration, such as sterile pyrogen-free water. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

As used herein, the term “pre-cancerous” refers to a condition that is not malignant, but is likely to become malignant if left untreated.

The term “subject” as used herein refers to an animal. Preferably the animal is a mammal. More preferably the mammal is a human. A subject also refers to, for example, dogs, cats, horses, cows, pigs, guinea pigs, fish, birds and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers and/or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; cyclodextrins such as alpha-(α), beta-(B) and gamma-(γ) cyclodextrins; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al, and WO 98/43,650 by Montgomery, all of which are incorporated herein by reference). A discussion of pulmonary delivery of antibiotics is also found in U.S. Pat. No. 6,014,969, incorporated herein by reference.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### SYNTHETIC METHODS AND EXAMPLES

The compounds and processes of the present invention will be better understood in connection with the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

TABLE 1-A

| | |
|---|---|
| | (II) |
| 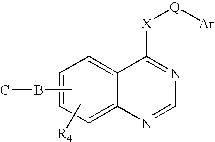 | |

SECTION 1:

| Compound # | Structure |
|---|---|
| 1 | 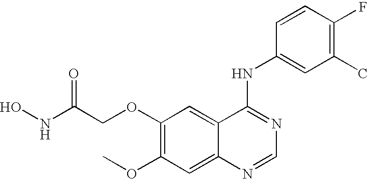 |
| 2 | 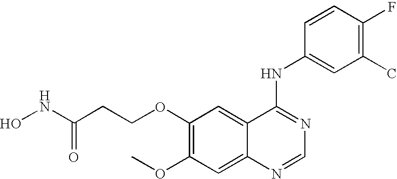 |
| 3 | 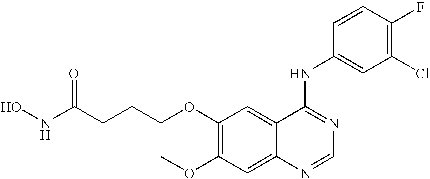 |
| 4 | 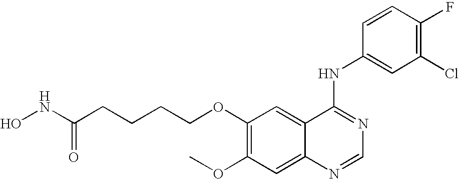 |
| 5 | 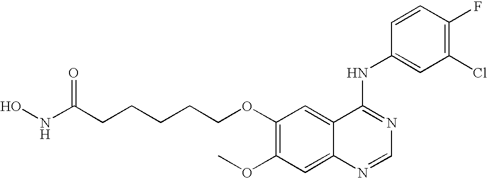 |
| 6 | 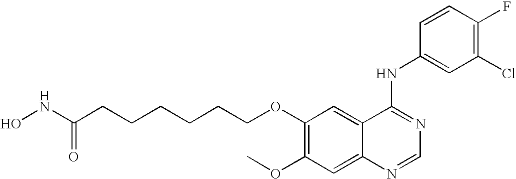 |
| 7 | 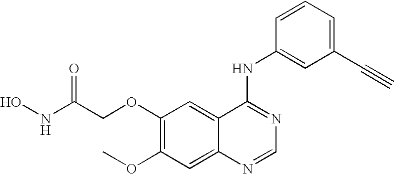 |
| 8 | 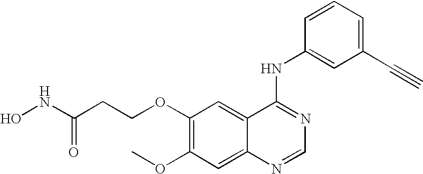 |
| 9 | 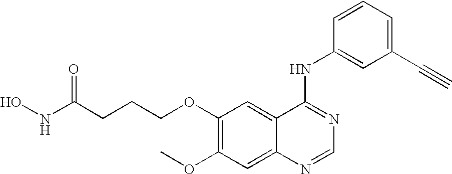 |
| 10 | 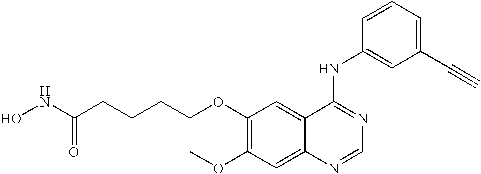 |
| 11 | 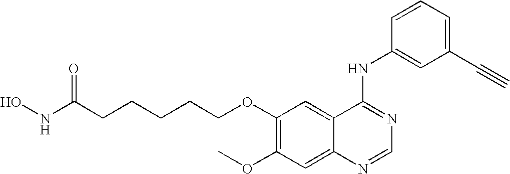 |
| 12 | 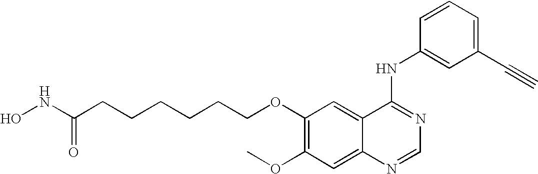 |
| 13 | 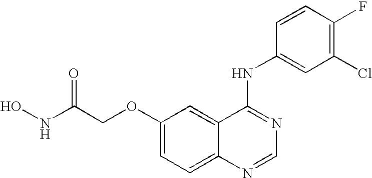 |
| 14 | 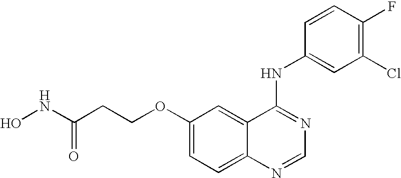 |
| 15 | 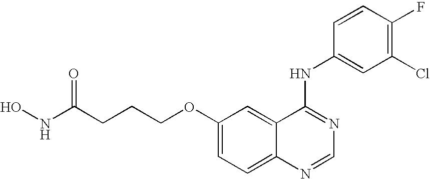 |
| 16 | 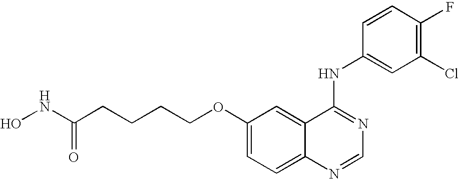 |
| 17 | 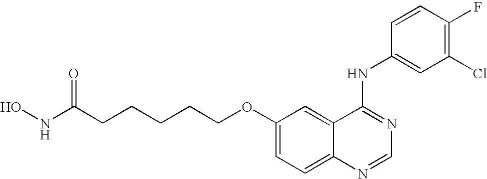 |
| 18 | 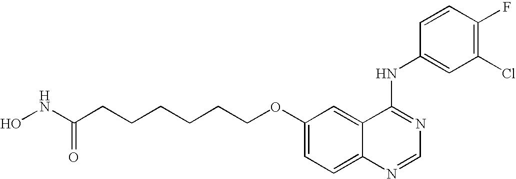 |
| 19 | 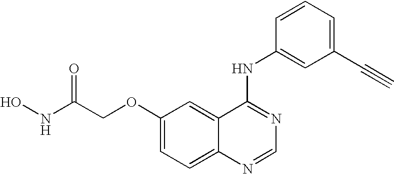 |
| 20 | 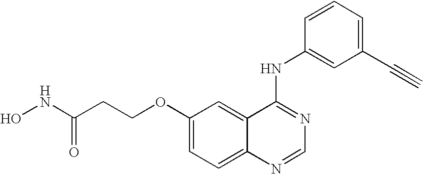 |
| 21 | 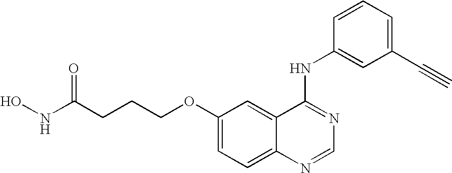 |
| 22 | 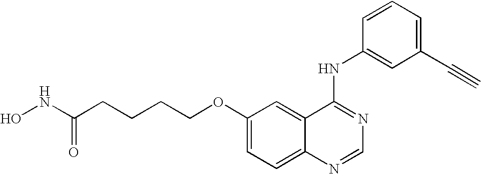 |
| 23 | 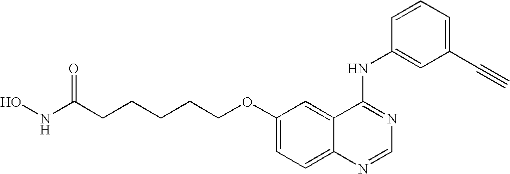 |
| 24 | 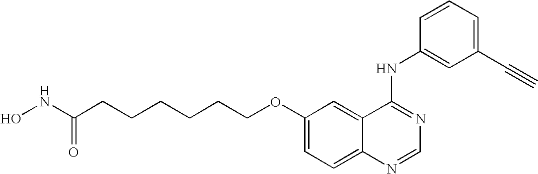 |
| 25 | 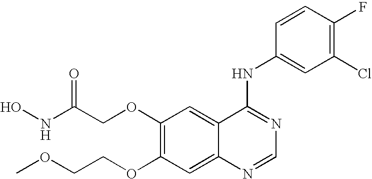 |
| 26 | 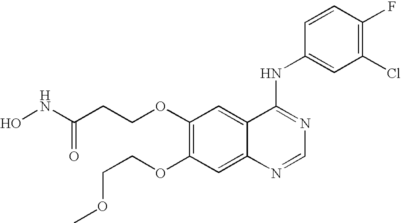 |
| 27 | 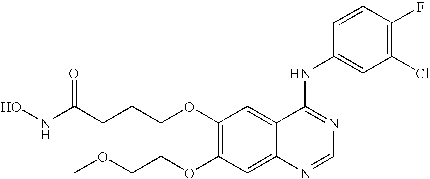 |
| 28 | 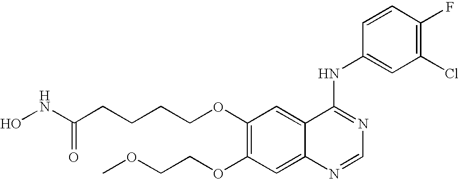 |
| 29 | 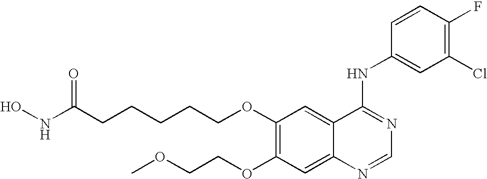 |
| 30 | 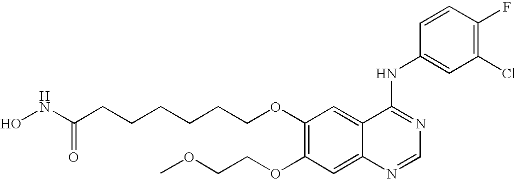 |
| 31 | 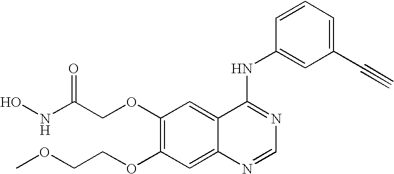 |
| 32 | 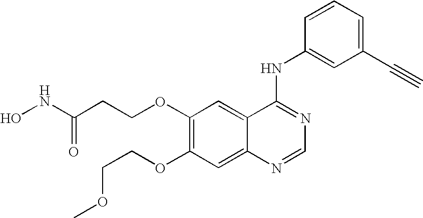 |
| 33 | 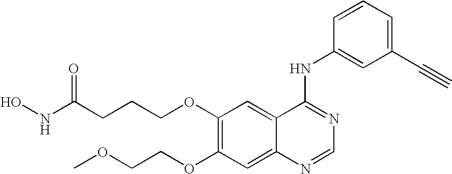 |
| 34 | 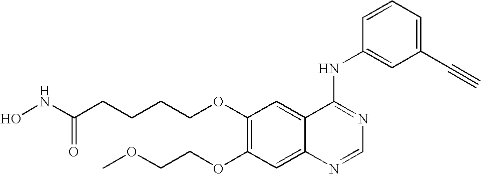 |
| 35 | 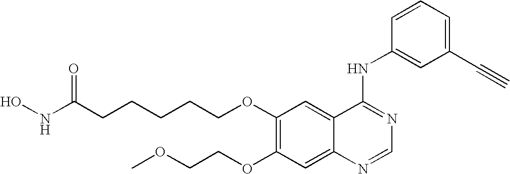 |
| 36 | 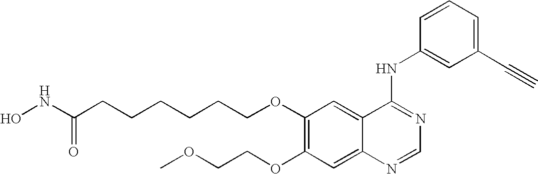 |
| 37 | 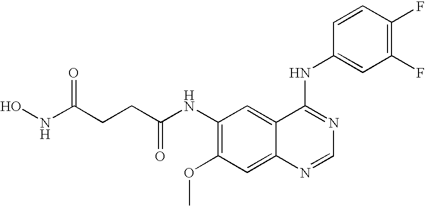 |
| 38 | 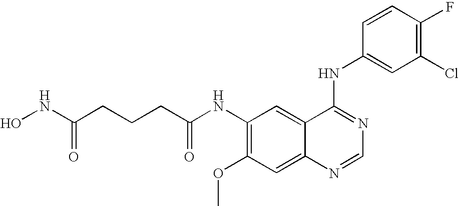 |
| 39 | 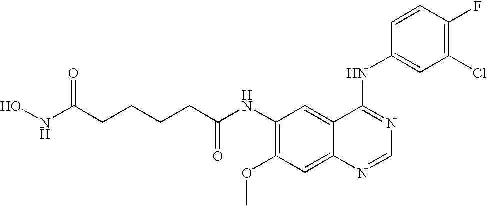 |
| 40 | 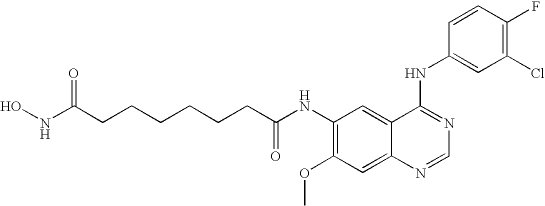 |
| 41 | 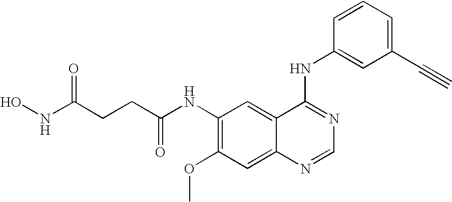 |
| 42 | 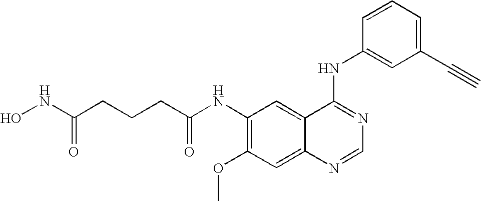 |
| 43 | 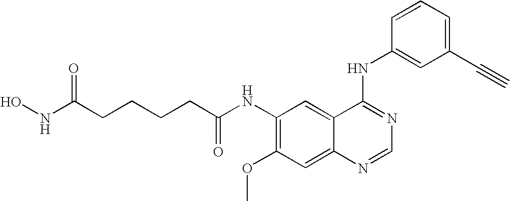 |
| 44 | 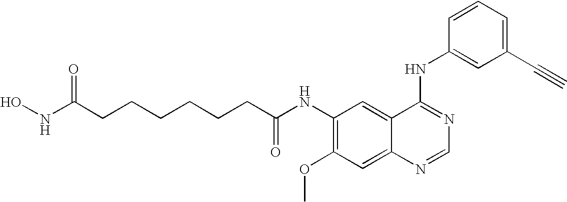 |
| 45 | 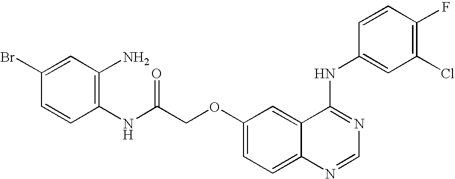 |
| 46 | 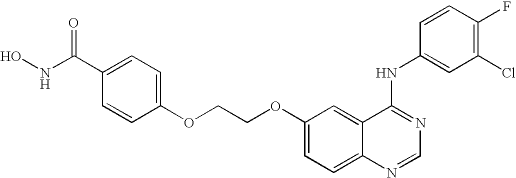 |
| 47 | 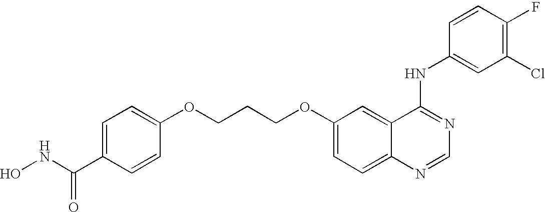 |
| 48 | 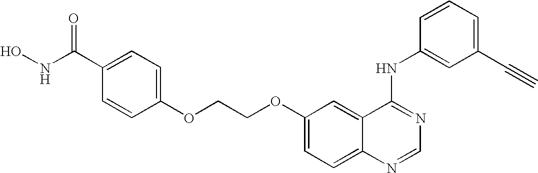 |
| 49 | 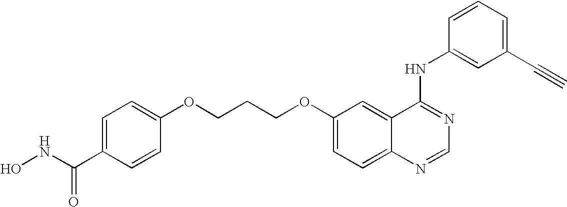 |
| 50 | 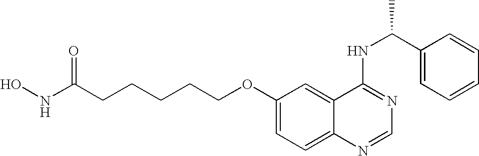 |
| 51 | 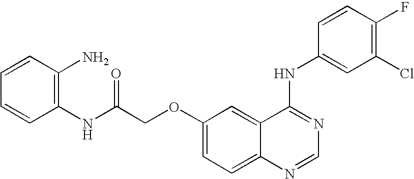 |
| 52 | 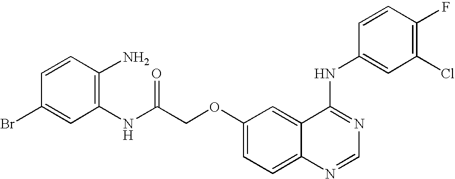 |
| 53 | 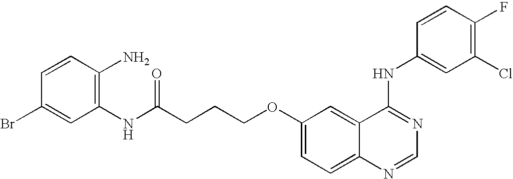 |
| 54 | 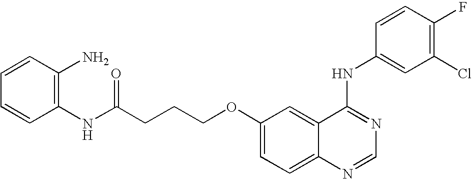 |
| 55 | 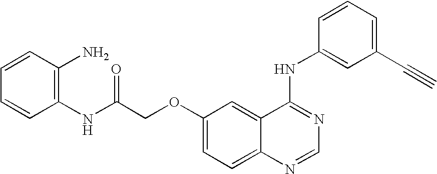 |
| 56 | 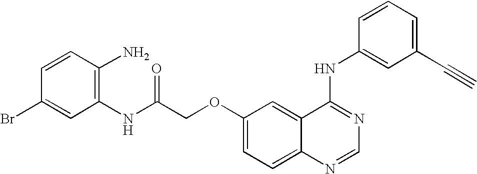 |
| 57 | 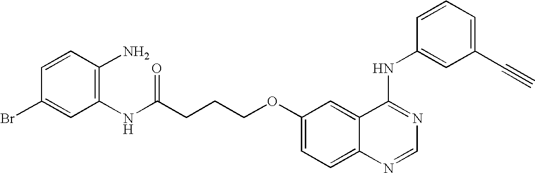 |
| 58 | 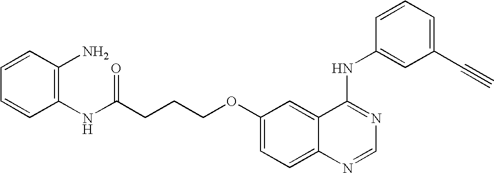 |
| 59 | 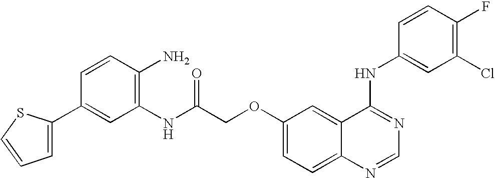 |
| 60 | 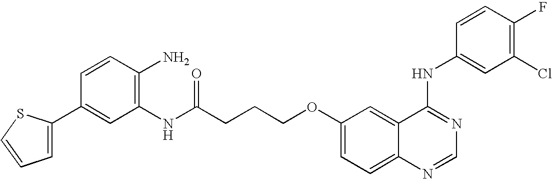 |
| 61 | 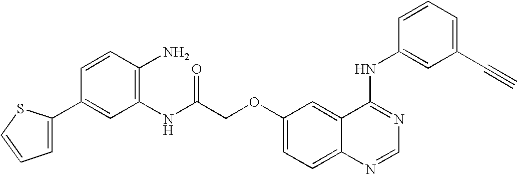 |
| 62 | 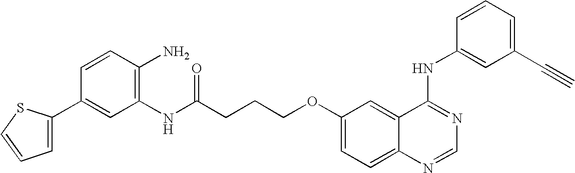 |
| 63 | 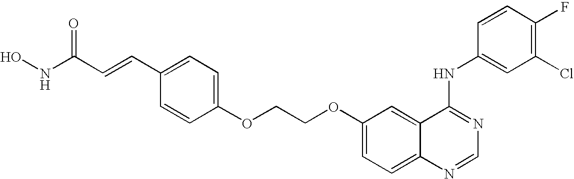 |
| 64 | 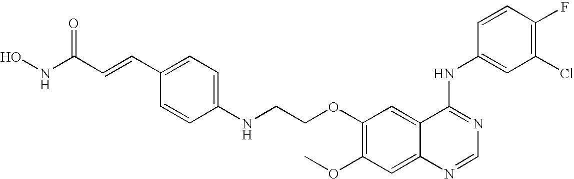 |
| 65 | 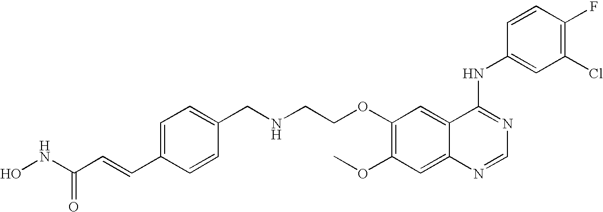 |
| 66 | 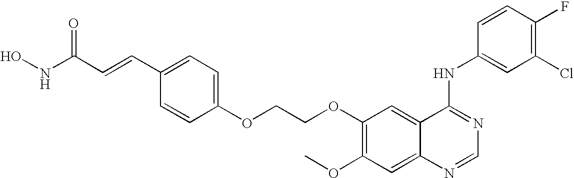 |
| 67 | 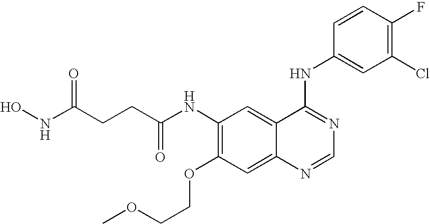 |
| 68 | 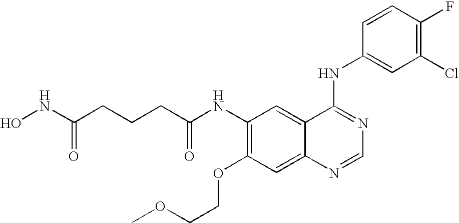 |
| 69 | 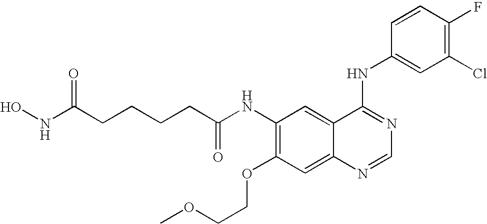 |
| 70 | 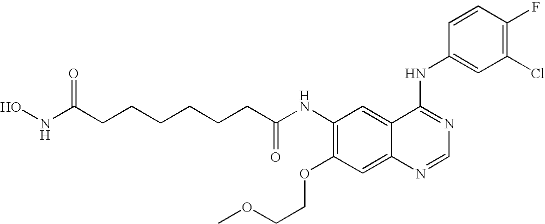 |
| 71 | 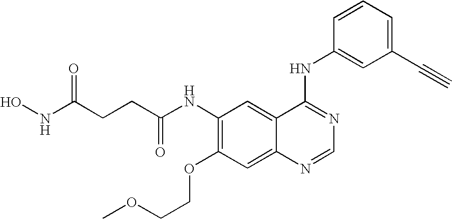 |
| 72 | 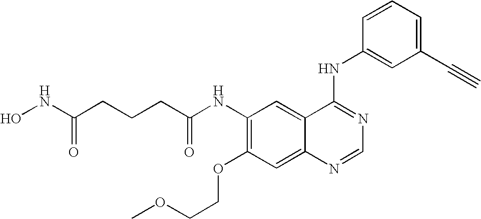 |
| 73 | 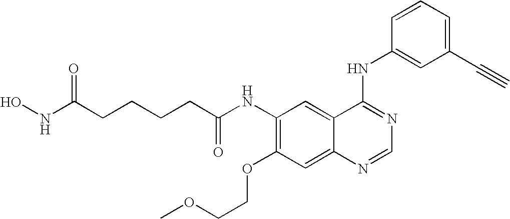 |
| 74 | 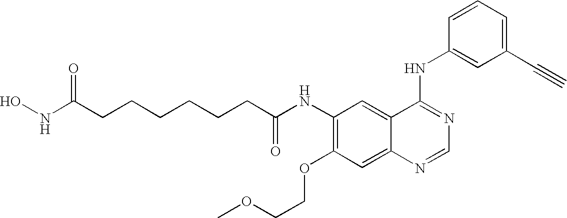 |
| 75 | 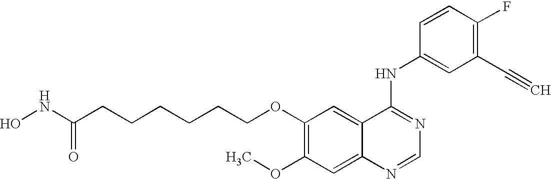 |
| 76 | 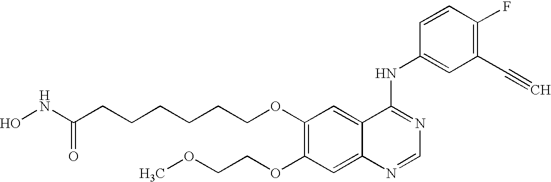 |
| 77 | 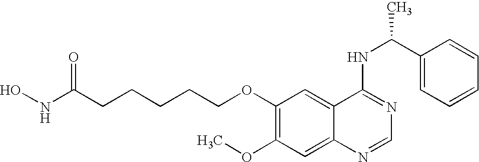 |
| 78 | 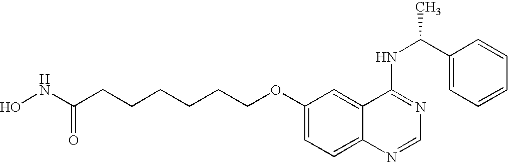 |
| 79 | 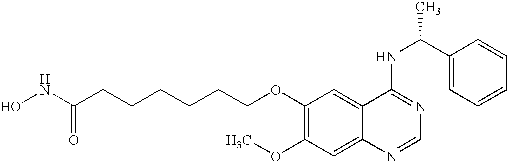 |
| 80 | 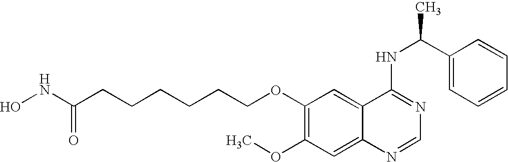 |
| 81 | 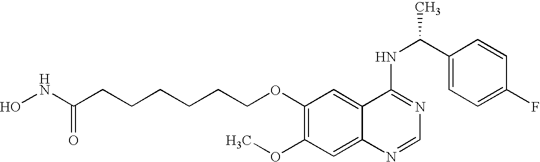 |
| 82 | 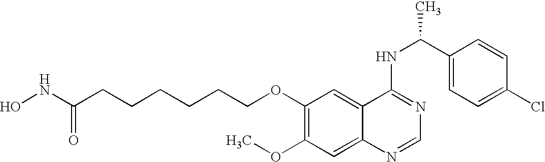 |
| 83 | 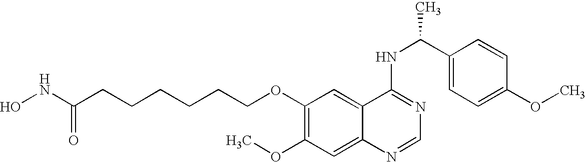 |
| 84 | 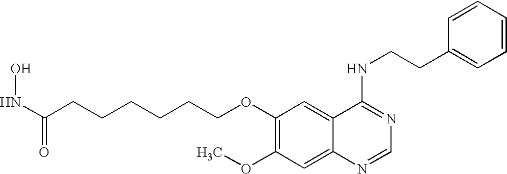 |
| 85 | 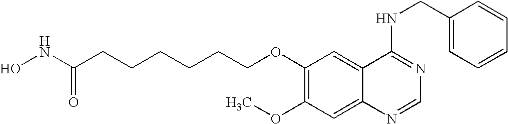 |
| 86 | 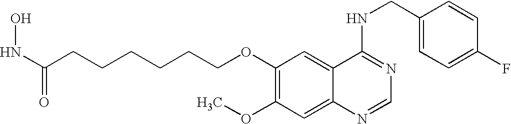 |
| 87 | 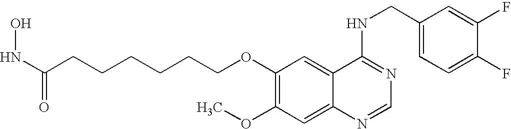 |
| 88 | 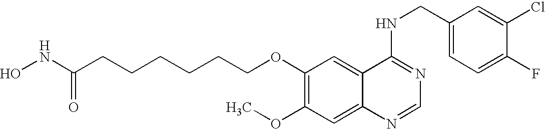 |
| 89 | 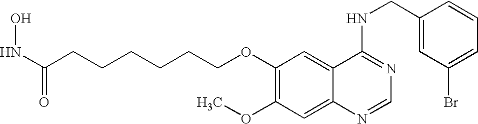 |
| 90 | 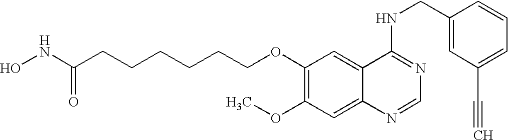 |
| 91 | 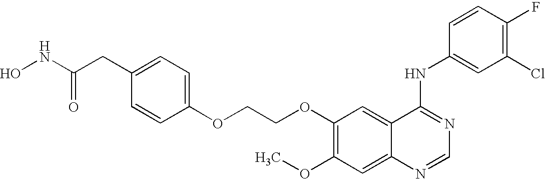 |
| 92 | 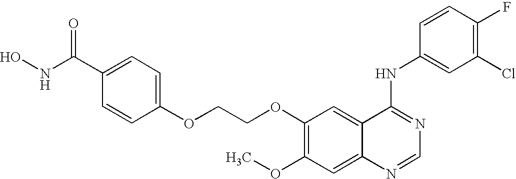 |
| 93 | 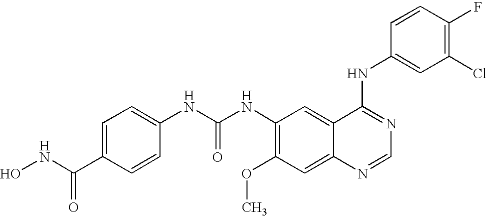 |
| 94 | 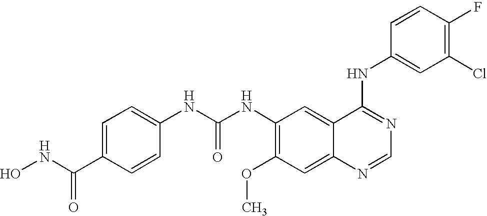 |
| 95 | 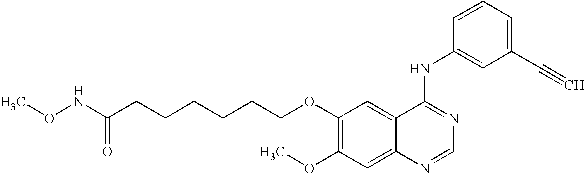 |
| 96 | 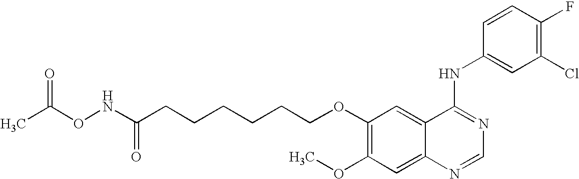 |
| 97 | 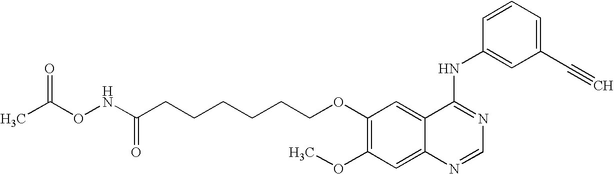 |
| 98 | 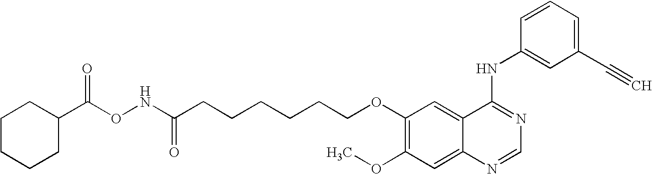 |
| 99 | 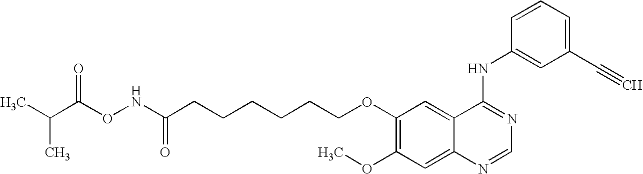 |
| 100 | 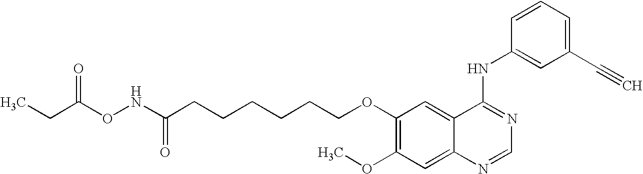 |
| 101 | 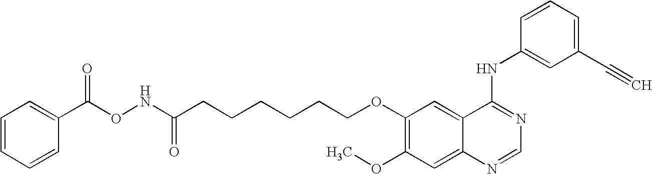 |
| 102 | 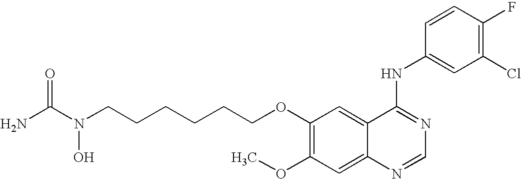 |
| 103 | 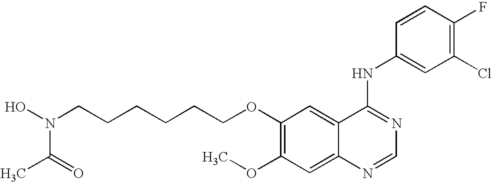 |
| 104 | 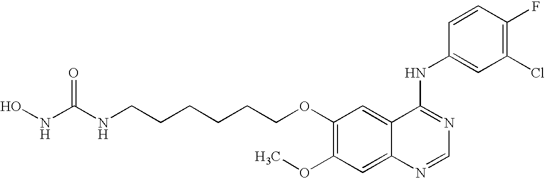 |
| 105 | 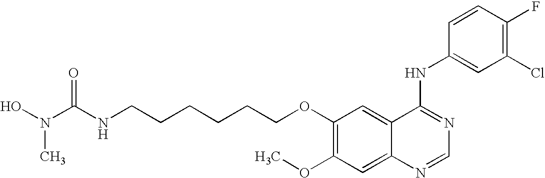 |
| 106 | 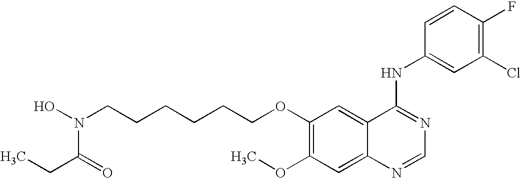 |
| 107 | 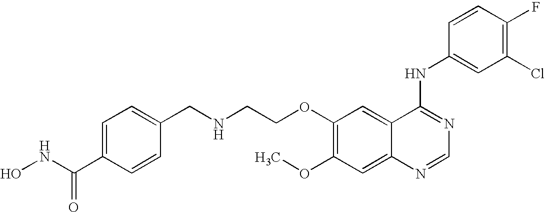 |
| 108 | 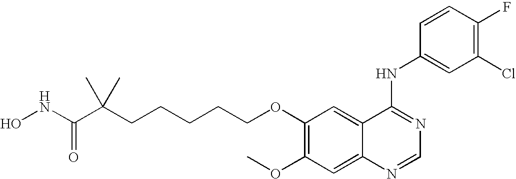 |
| 109 | 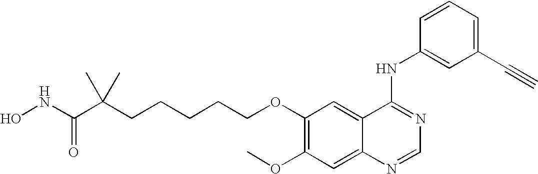 |
| 110 | 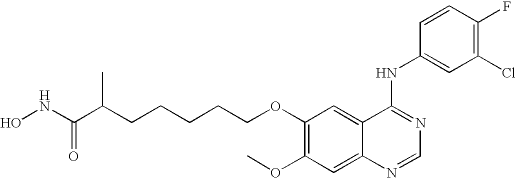 |
| 111 | 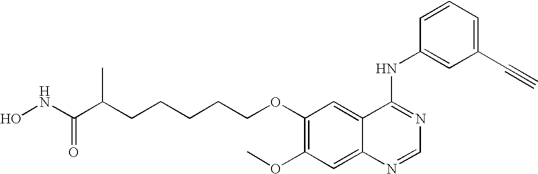 |
| 112 | 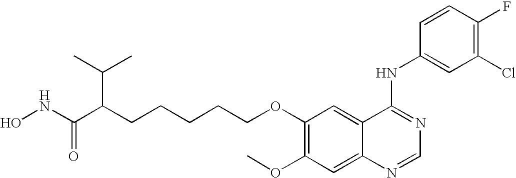 |
| 113 | 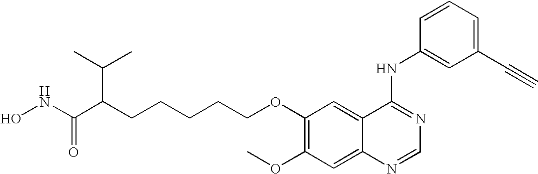 |
| 114 | 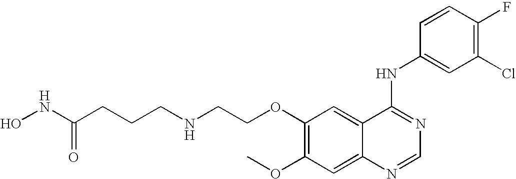 |
| 115 | 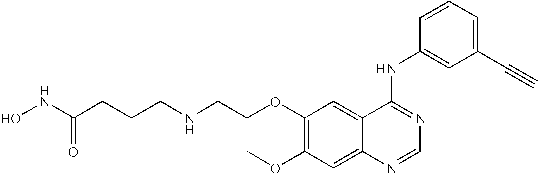 |
| 116 | 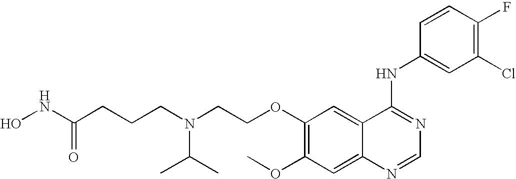 |
| 117 | 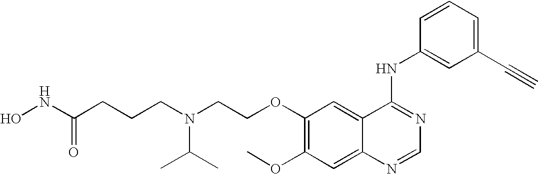 |
| 118 | 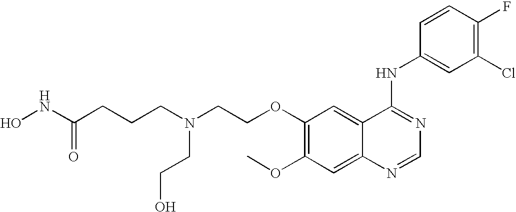 |
| 119 | 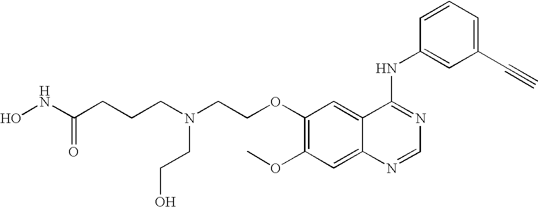 |
| 120 | 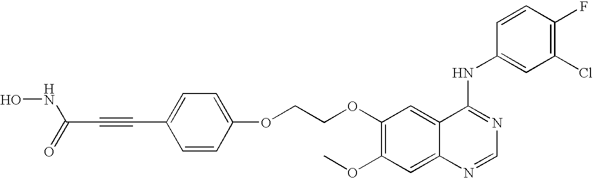 |
| 121 | 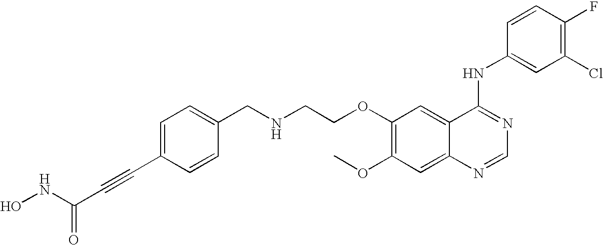 |
| 122 | 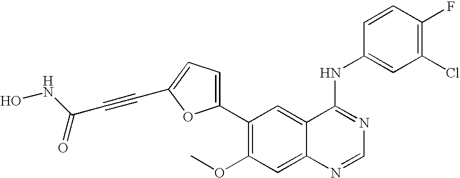 |
| 123 | 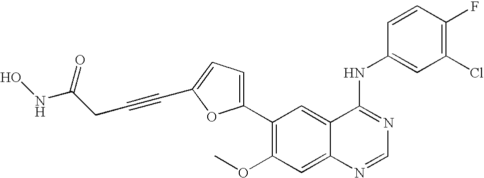 |
| 124 | 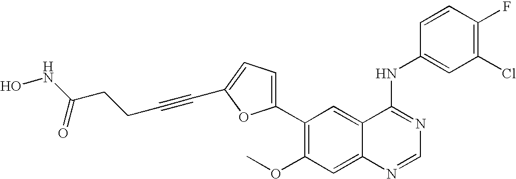 |
| 125 | 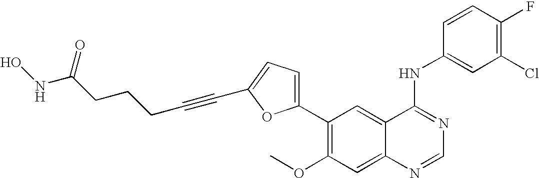 |
| 126 | 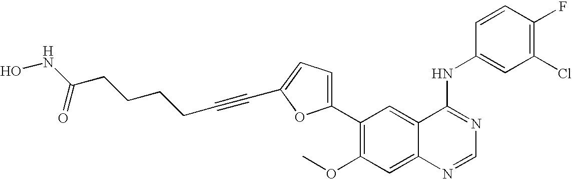 |
| 127 | 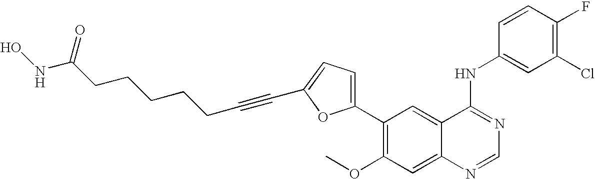 |
| 128 | 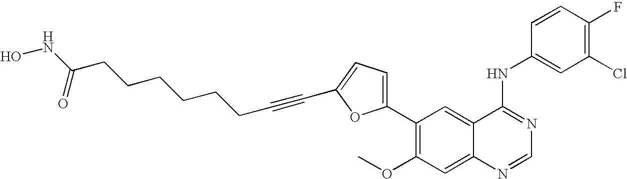 |
| 129 | 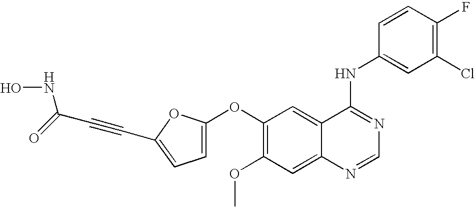 |
| 130 | 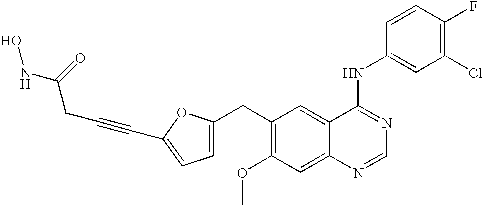 |
| 131 | 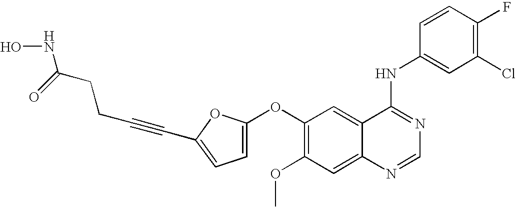 |
| 132 | 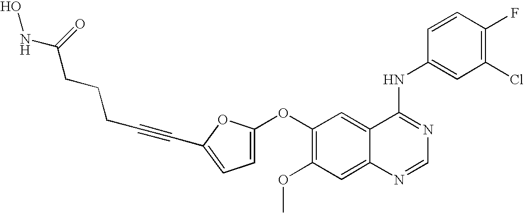 |
| 133 | 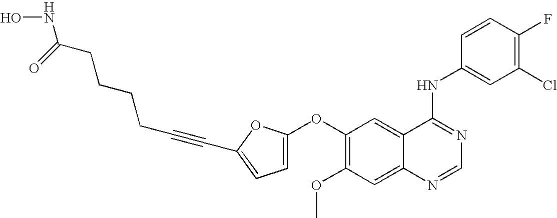 |
| 134 | 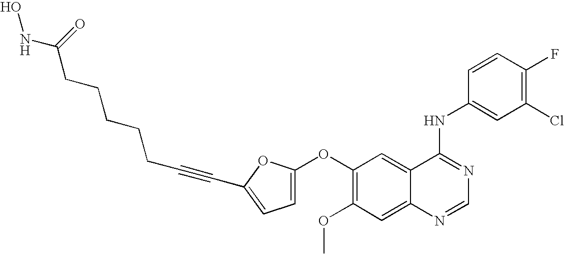 |
| 135 | 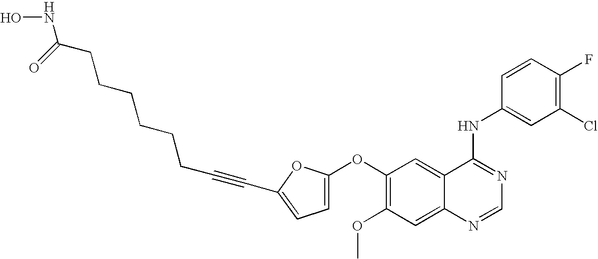 |
| 136 | 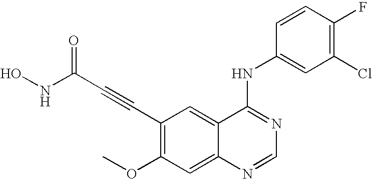 |
| 137 | 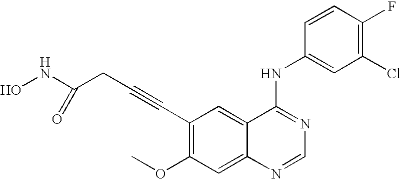 |
| 138 | 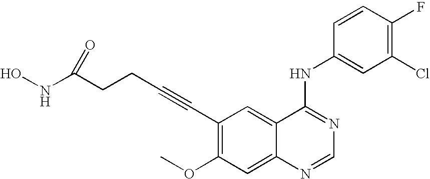 |
| 139 | 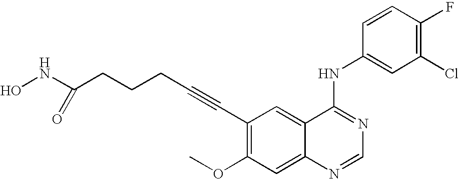 |
| 140 | 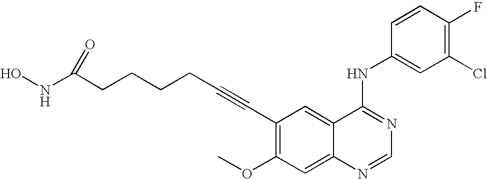 |
| 141 | 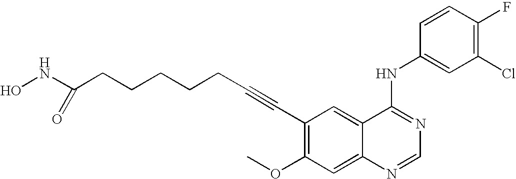 |
| 142 | 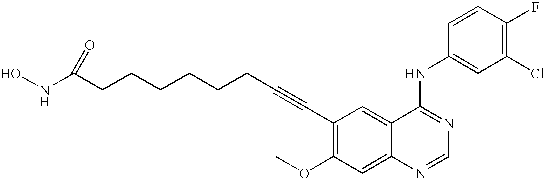 |
| 143 | 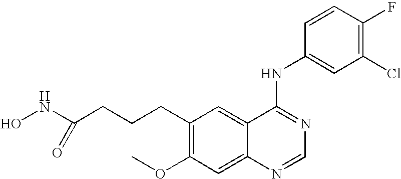 |
| 144 | 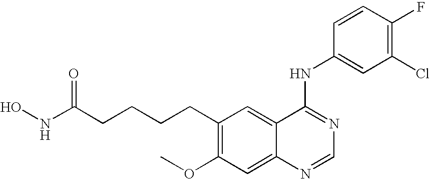 |
| 145 | 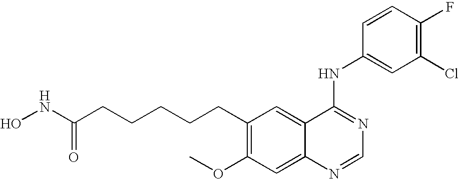 |
| 146 | 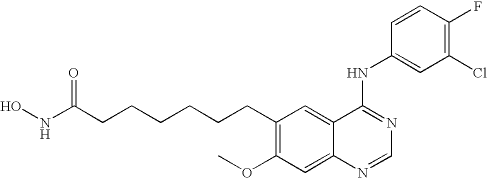 |
| 147 | 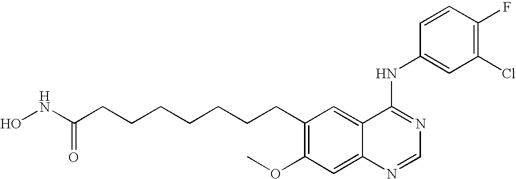 |
| 148 | 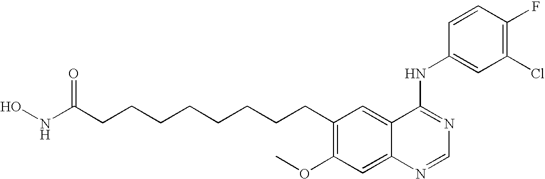 |
| 149 | 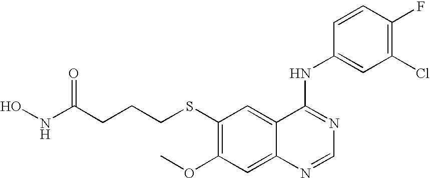 |
| 150 | 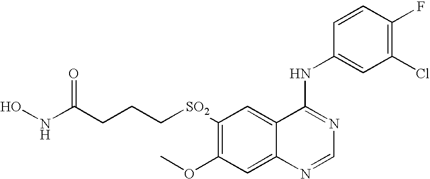 |
| 151 | 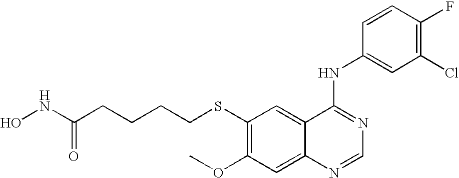 |
| 152 | 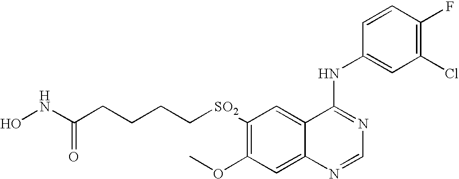 |
| 153 | 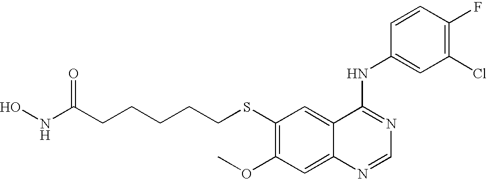 |
| 154 | 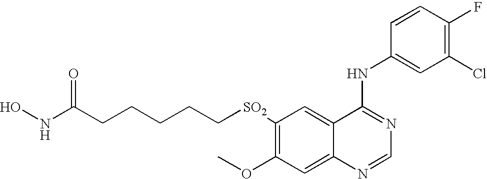 |
| 155 | 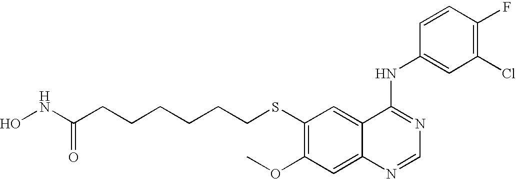 |
| 156 | 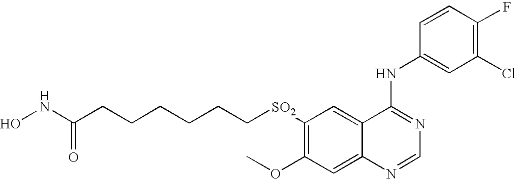 |
| 157 | 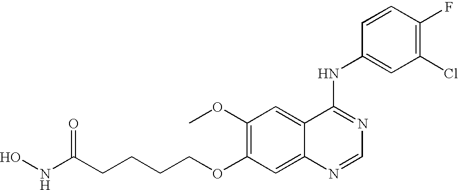 |
| 158 | 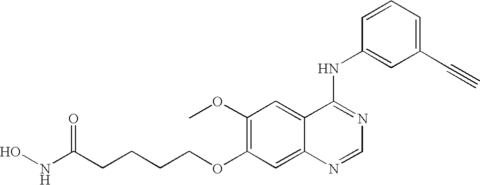 |
| 159 | 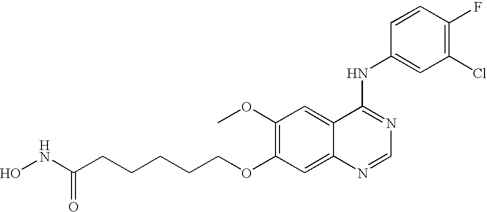 |
| 160 | 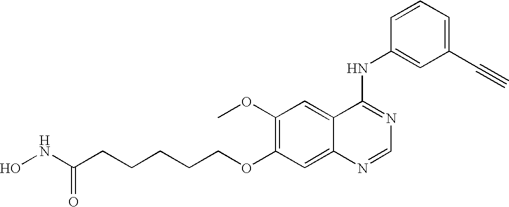 |
| 161 | 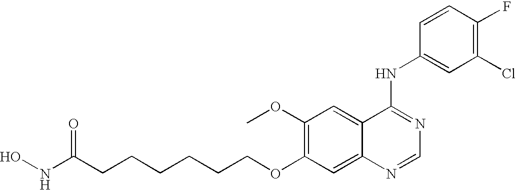 |
| 162 | 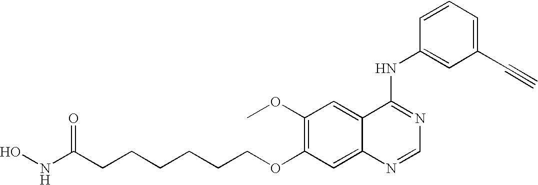 |
| 163 | 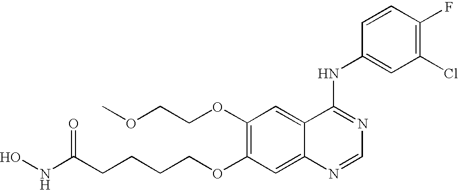 |
| 164 | 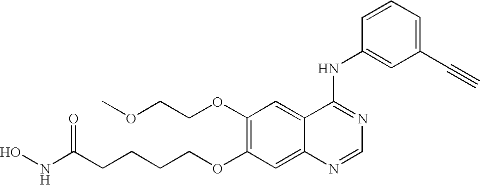 |
| 165 | 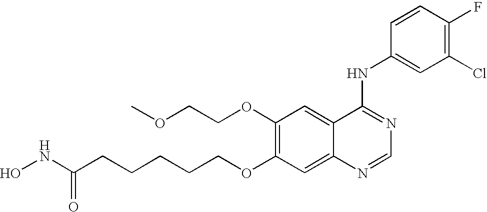 |
| 166 | 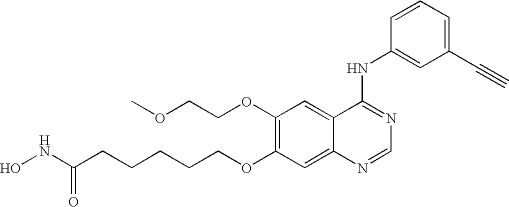 |
| 167 | 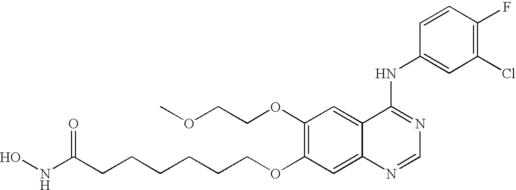 |
| 168 | 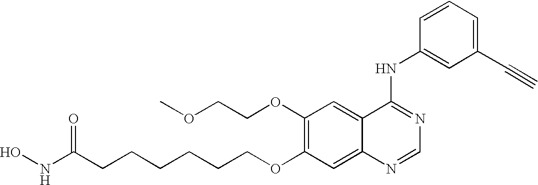 |
| 169 | 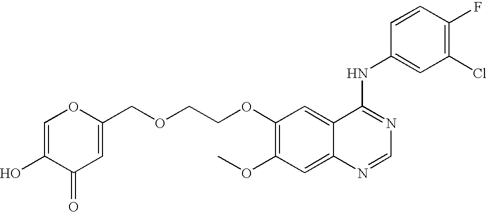 |
| 170 | 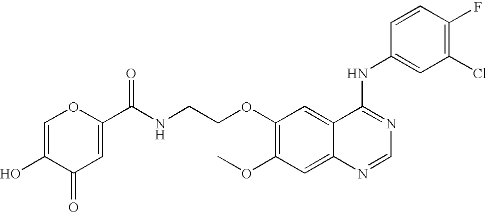 |
| 171 | 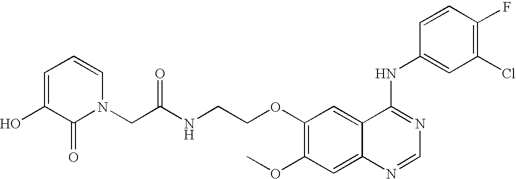 |
| 172 | 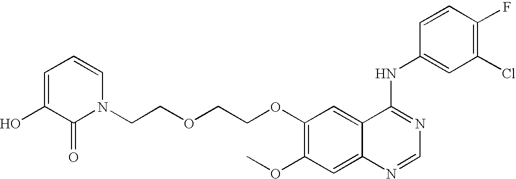 |
| 173 | 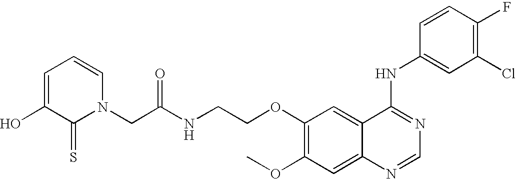 |
| 174 | 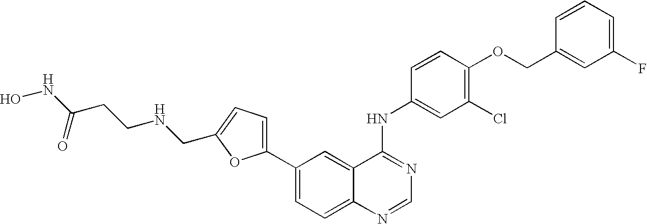 |
| 175 | 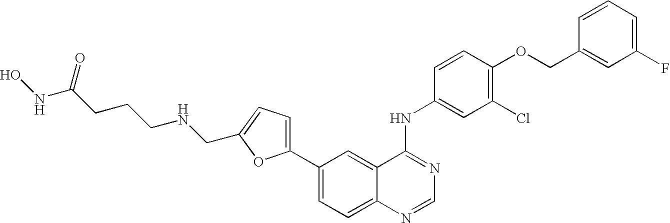 |
| 176 | 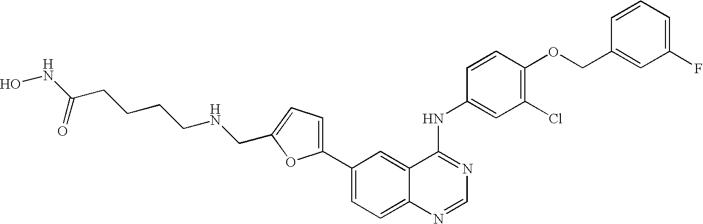 |
| 177 | 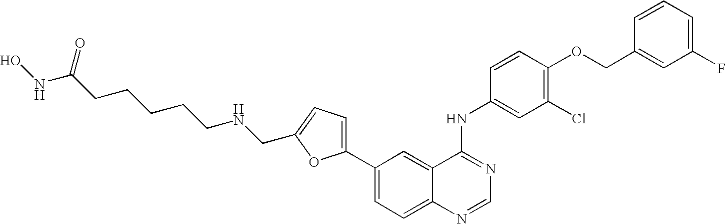 |
| 178 | 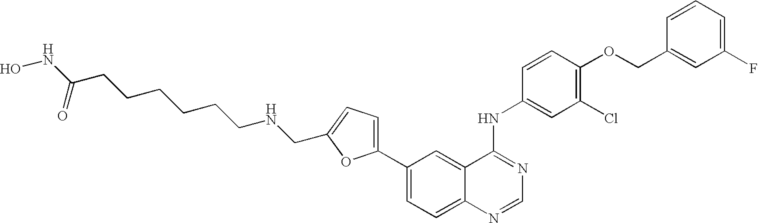 |
| 179 | 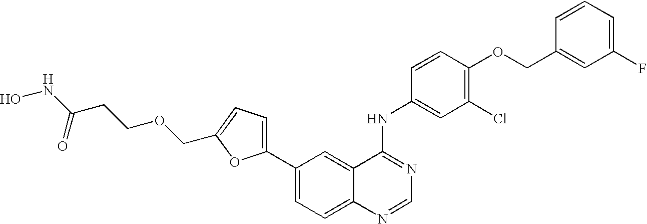 |
| 180 | 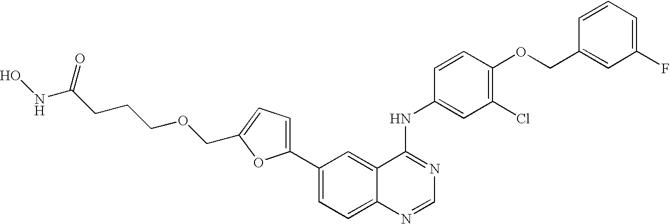 |
| 181 | 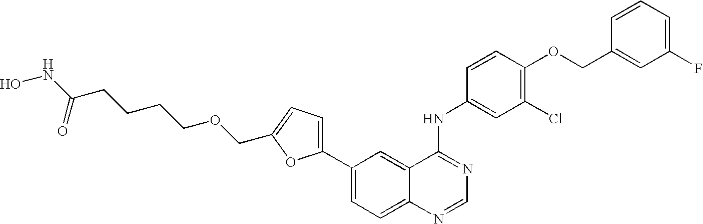 |
| 182 | 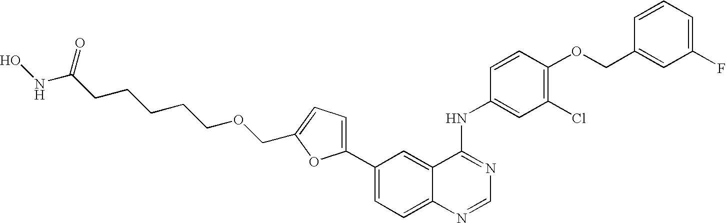 |
| 183 | 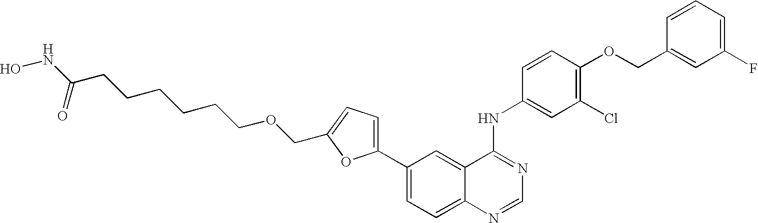 |
| 184 | 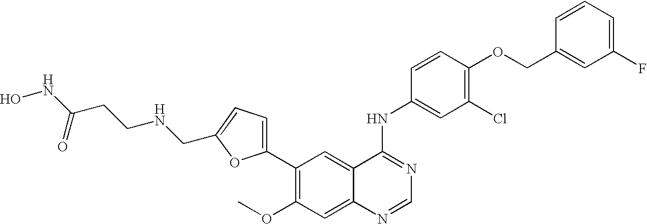 |
| 185 | 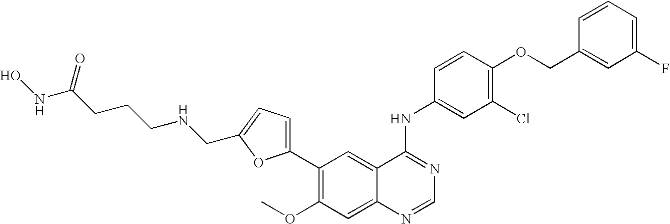 |
| 186 | 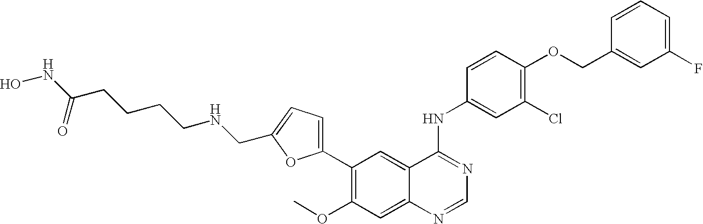 |
| 187 | 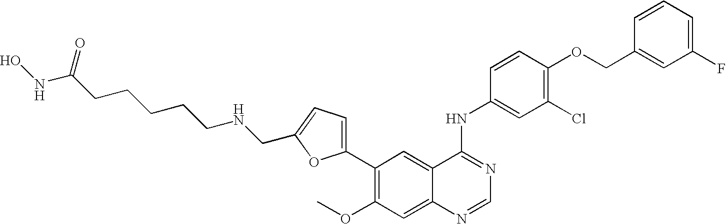 |
| 188 | 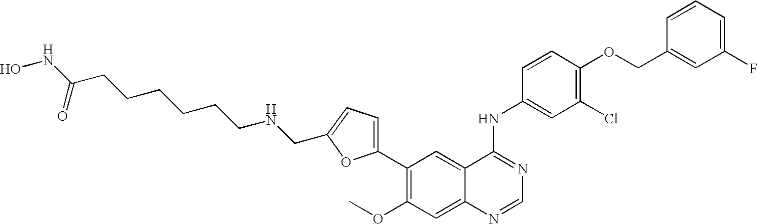 |
| 189 | 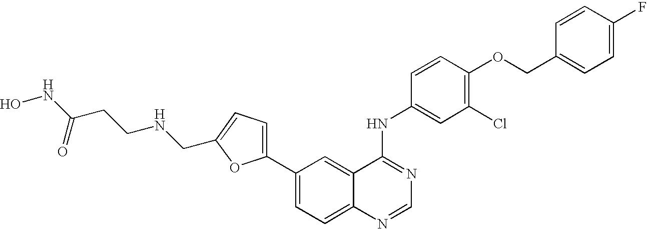 |
| 190 | 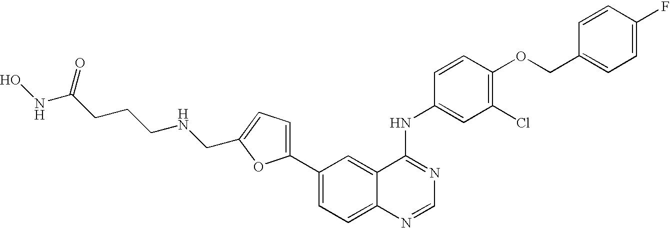 |
| 191 | 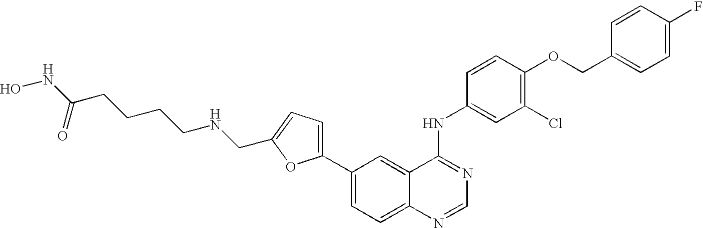 |
| 192 | 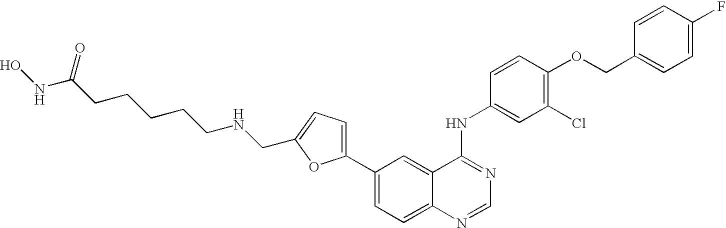 |
| 193 | 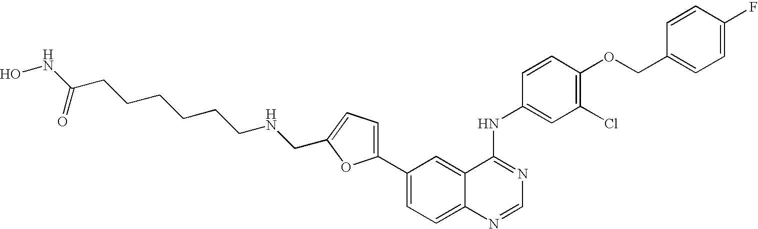 |
| 194 | 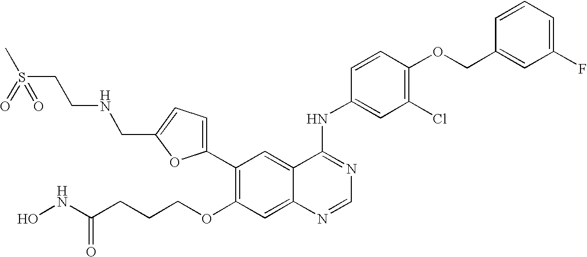 |
| 195 | 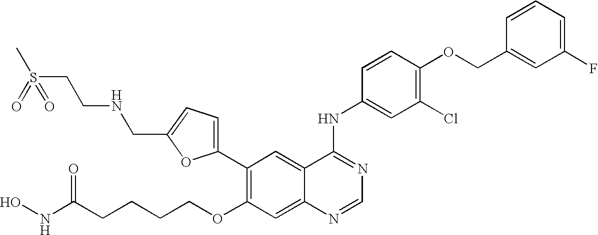 |
| 196 | 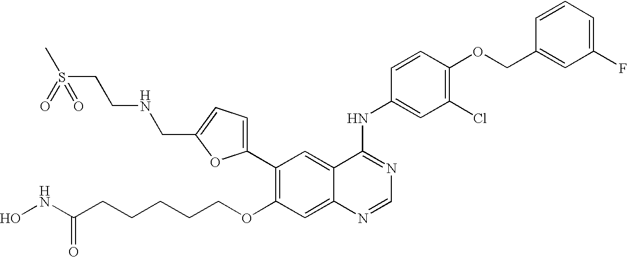 |
| 197 | 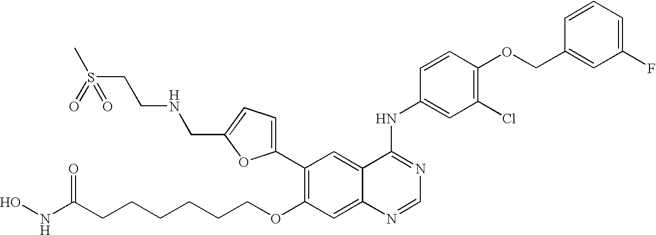 |
| 198 | 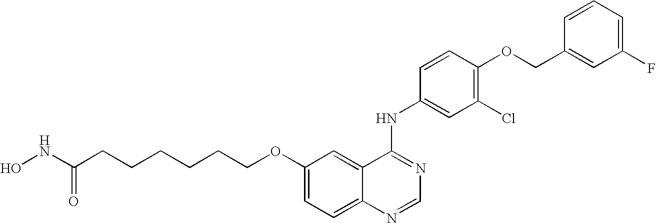 |
| 199 | 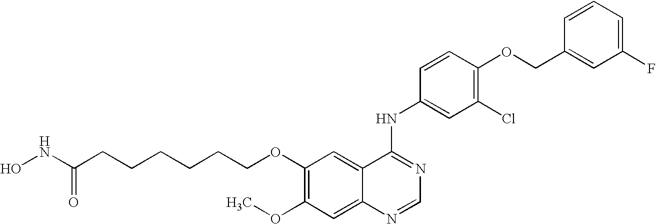 |
| 200 | 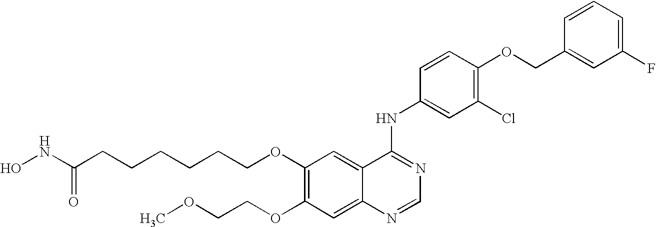 |
| 201 | 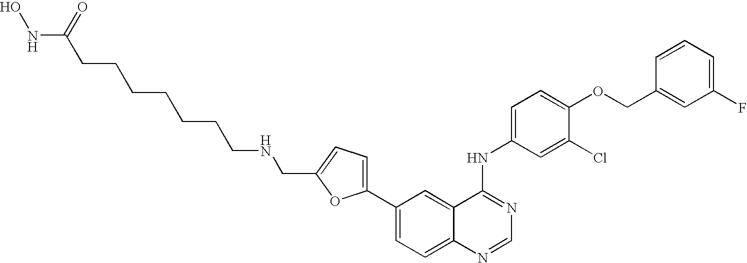 |
| 202 | 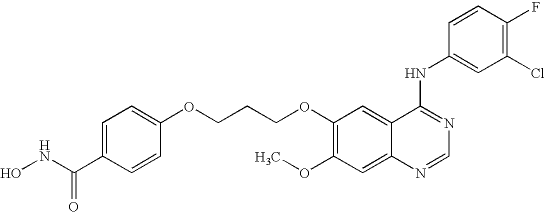 |
| 203 | 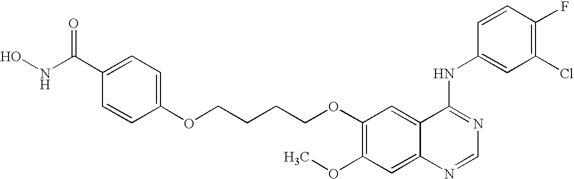 |
| 204 | 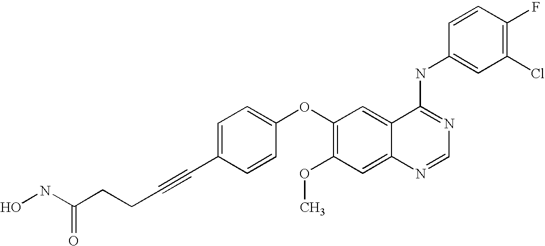 |
| 205 | 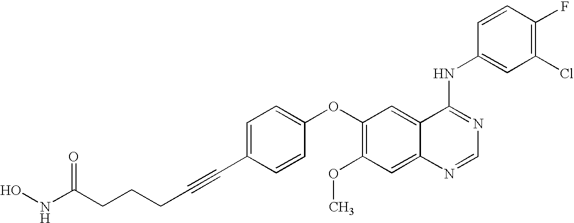 |
| 206 | 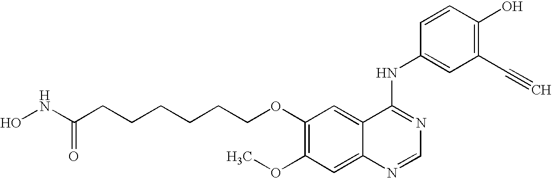 |
| 207 | 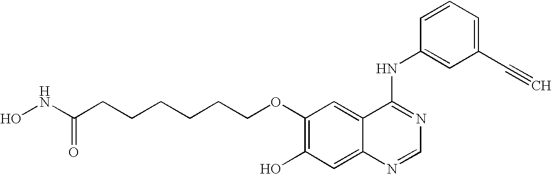 |
| 208 | 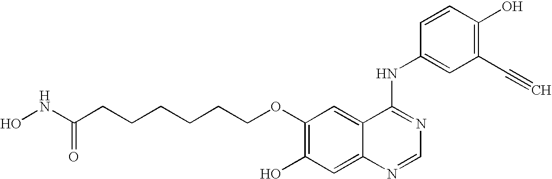 |
| 209 | 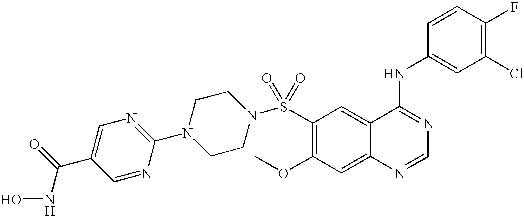 |
| 210 | 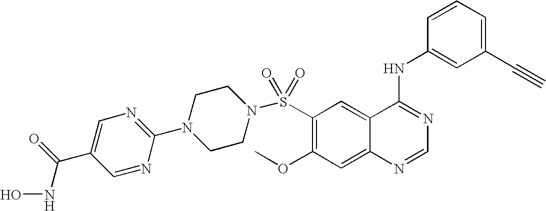 |
| 211 | 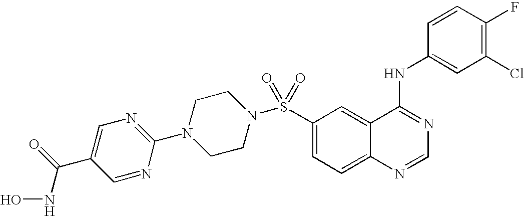 |
| 212 | 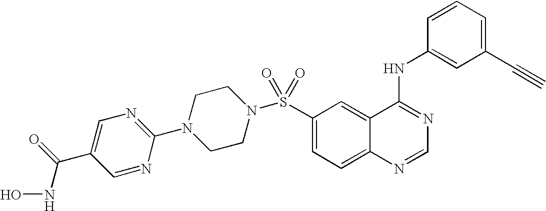 |

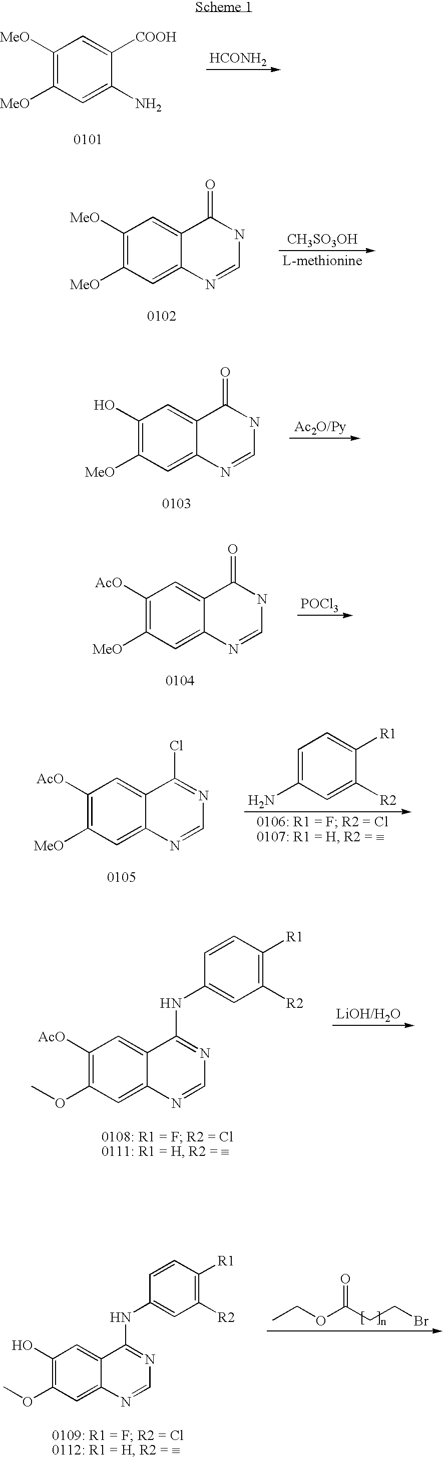

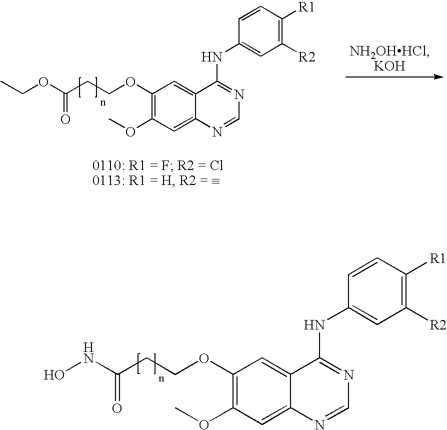

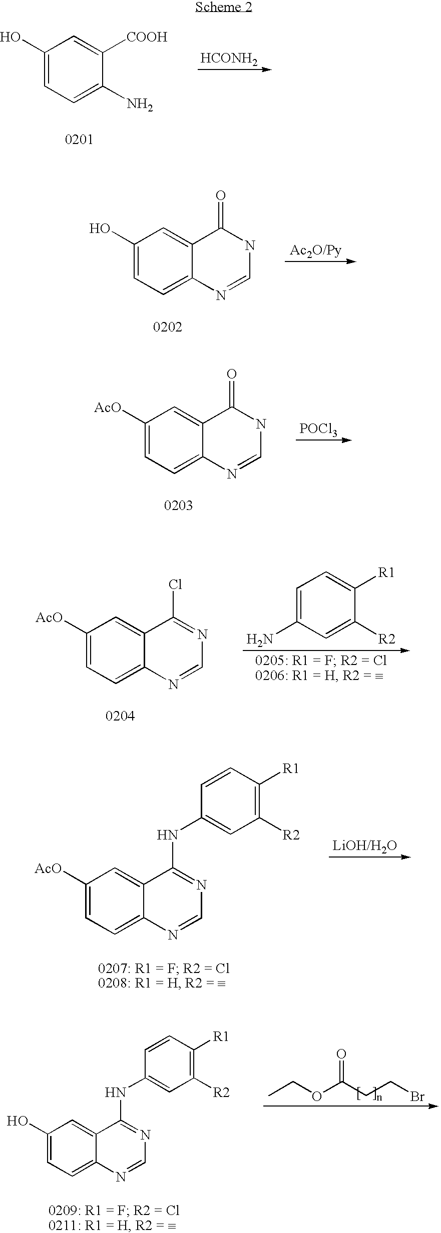

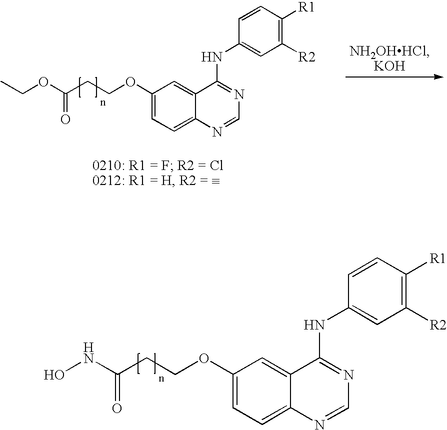

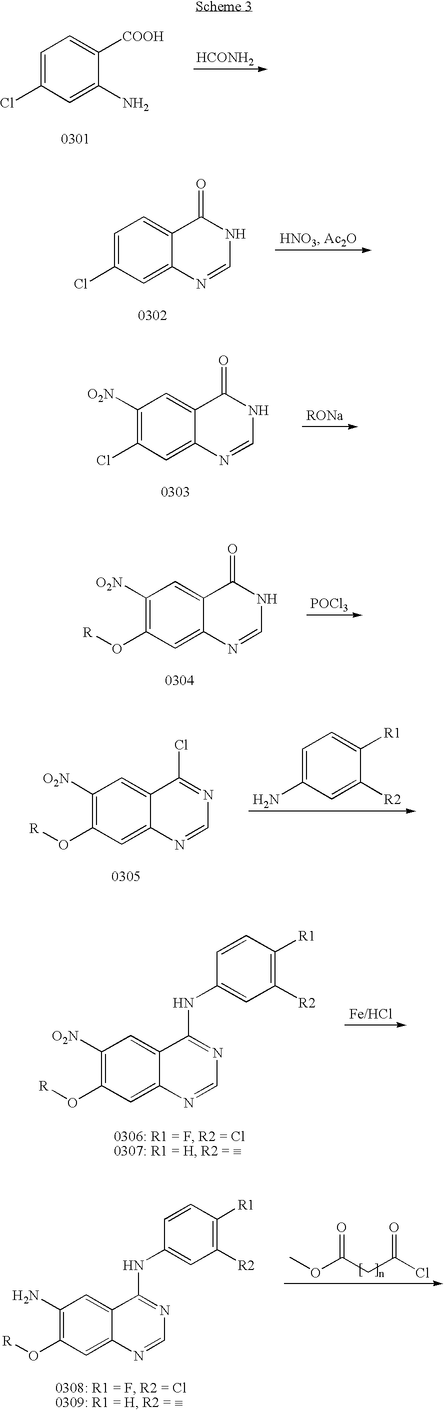

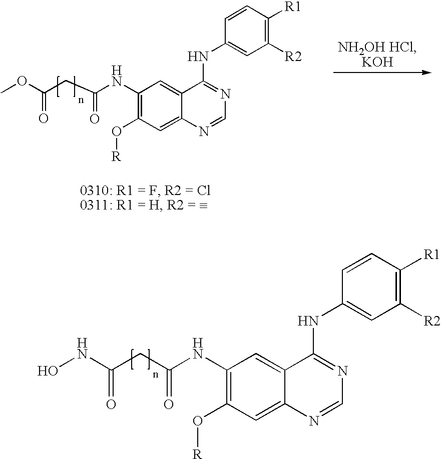

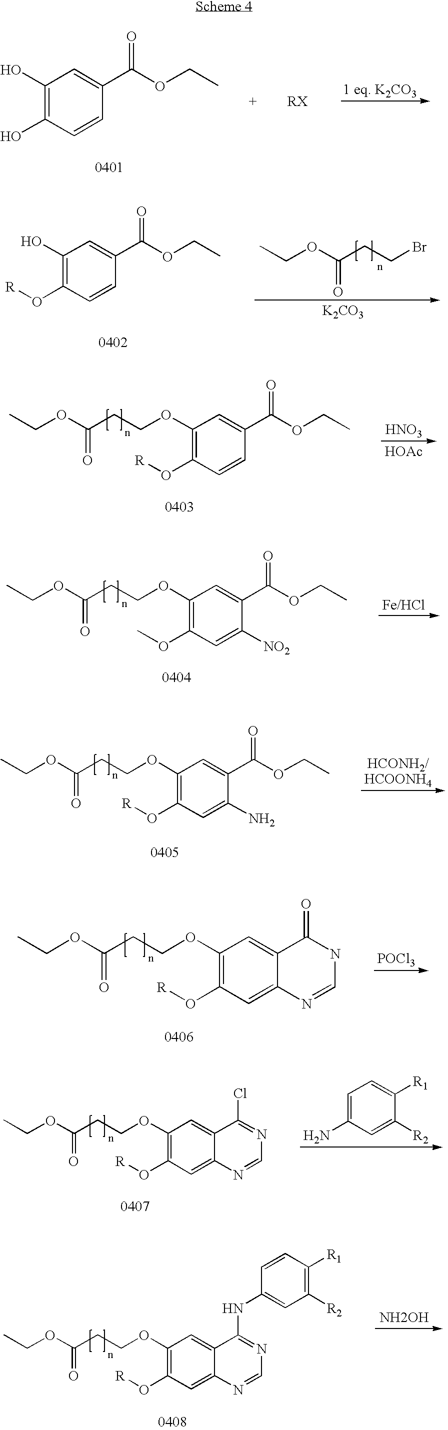

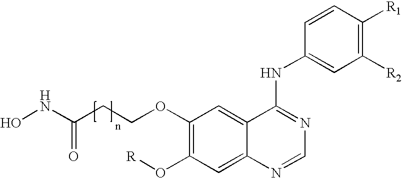

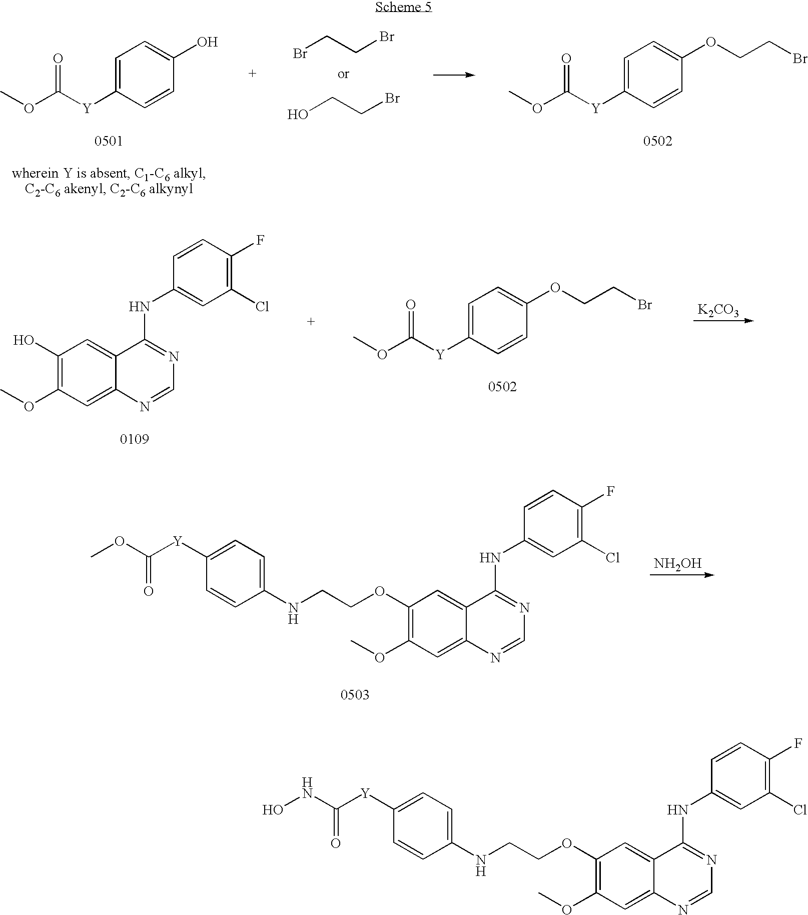

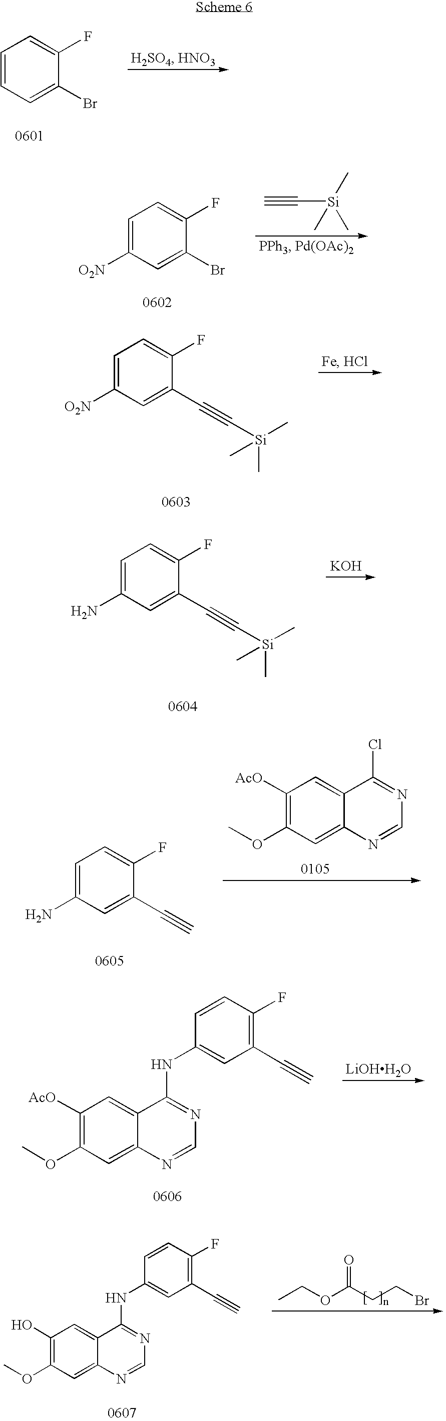

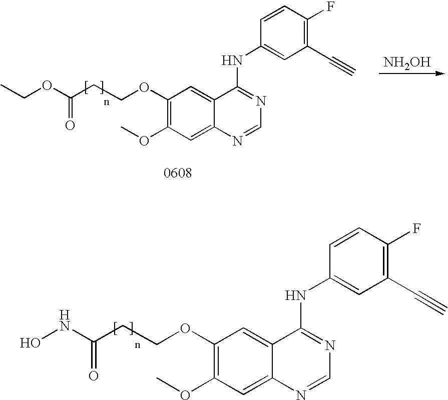

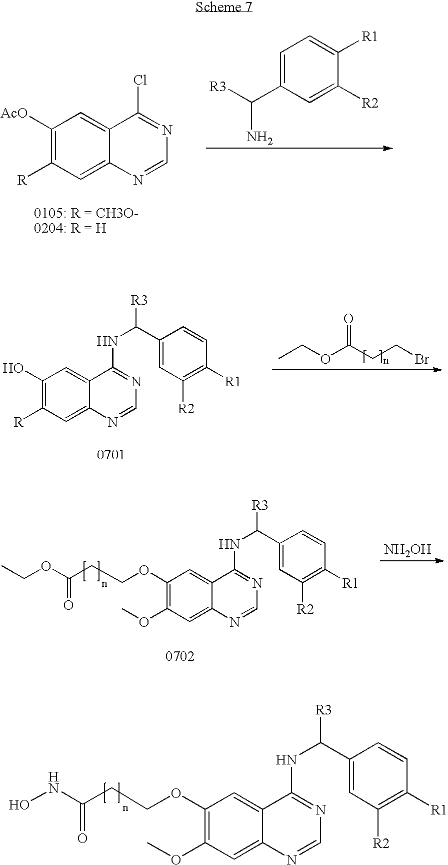

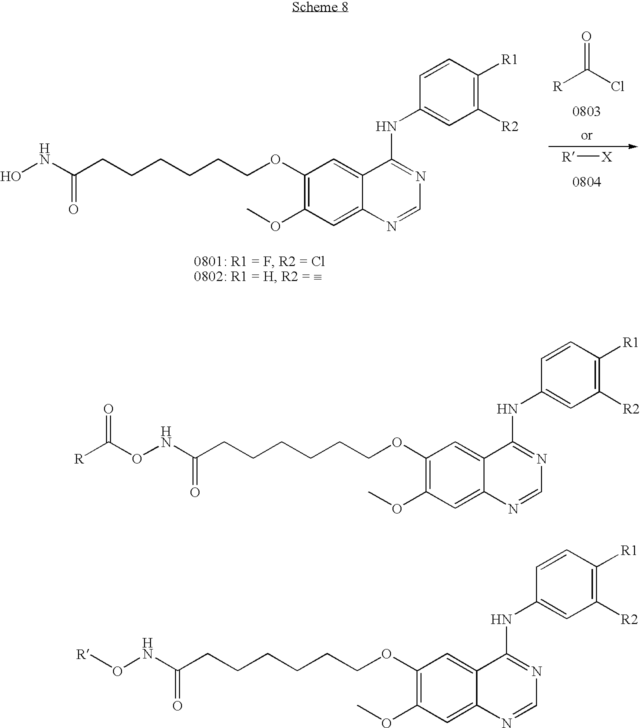

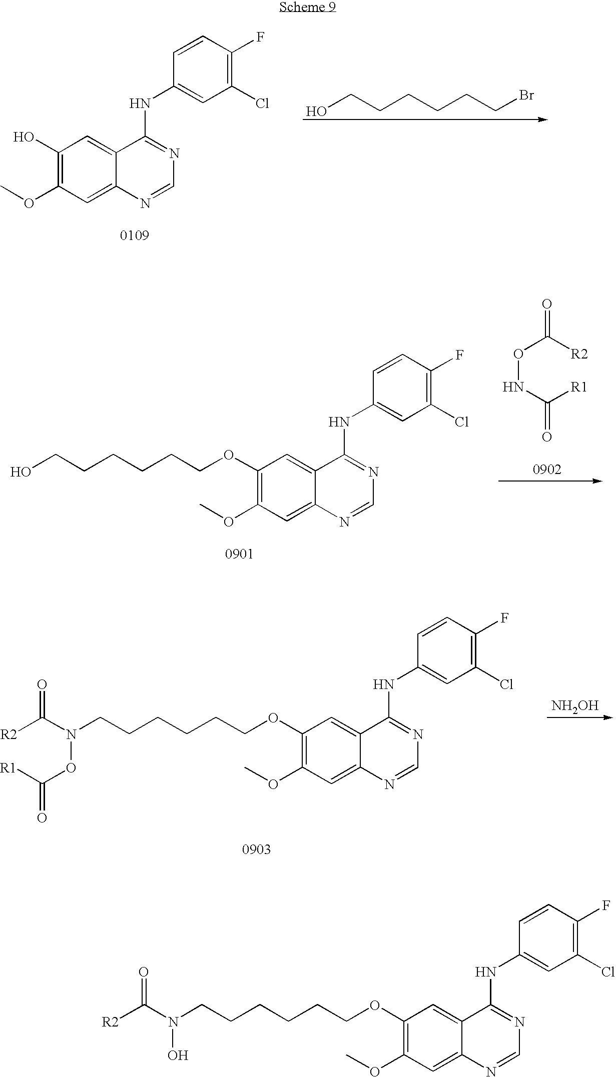

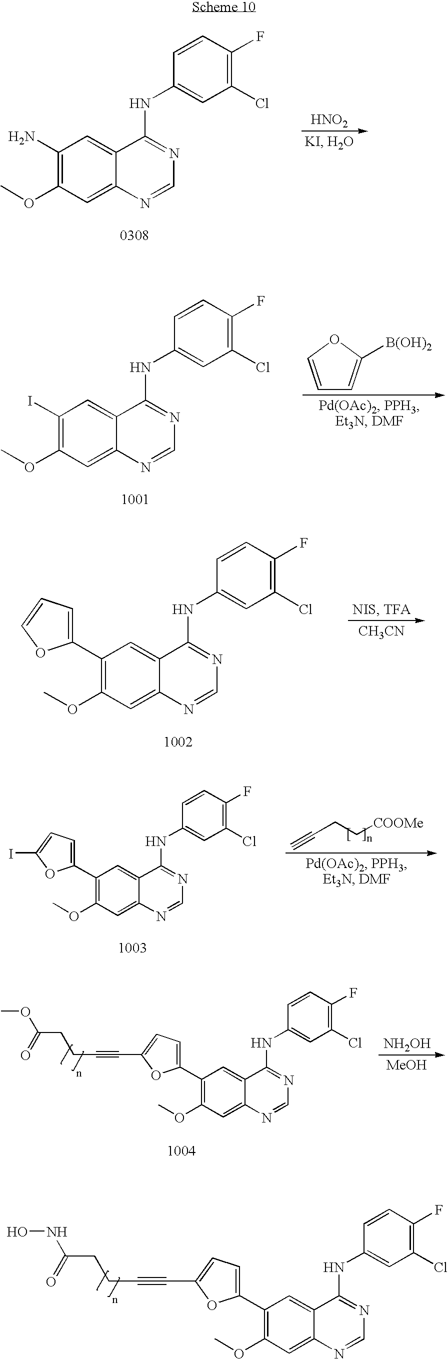

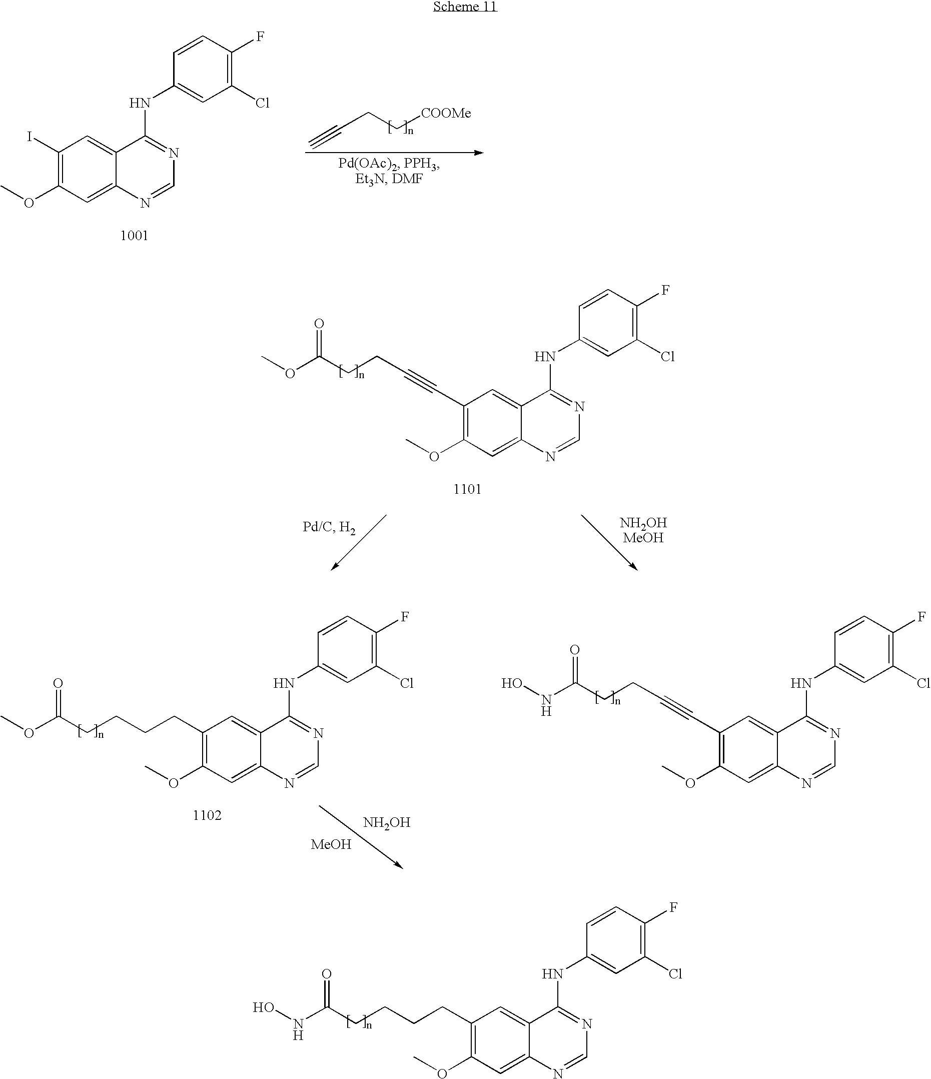

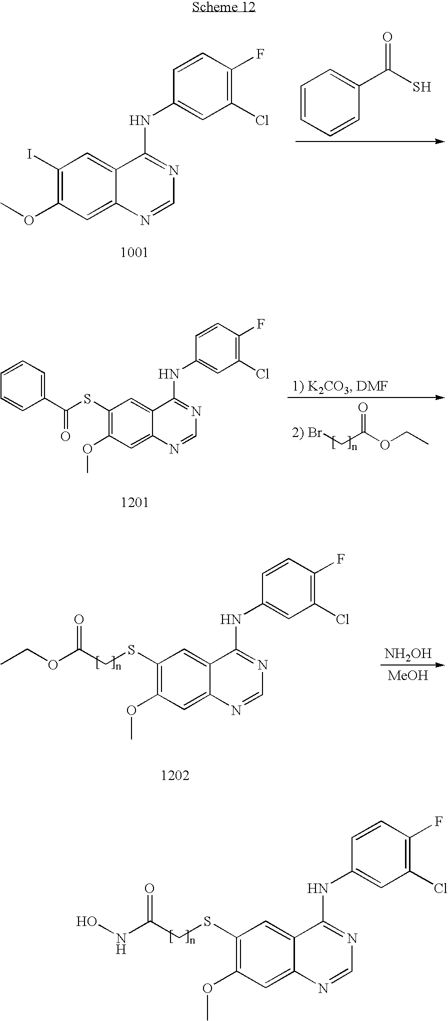

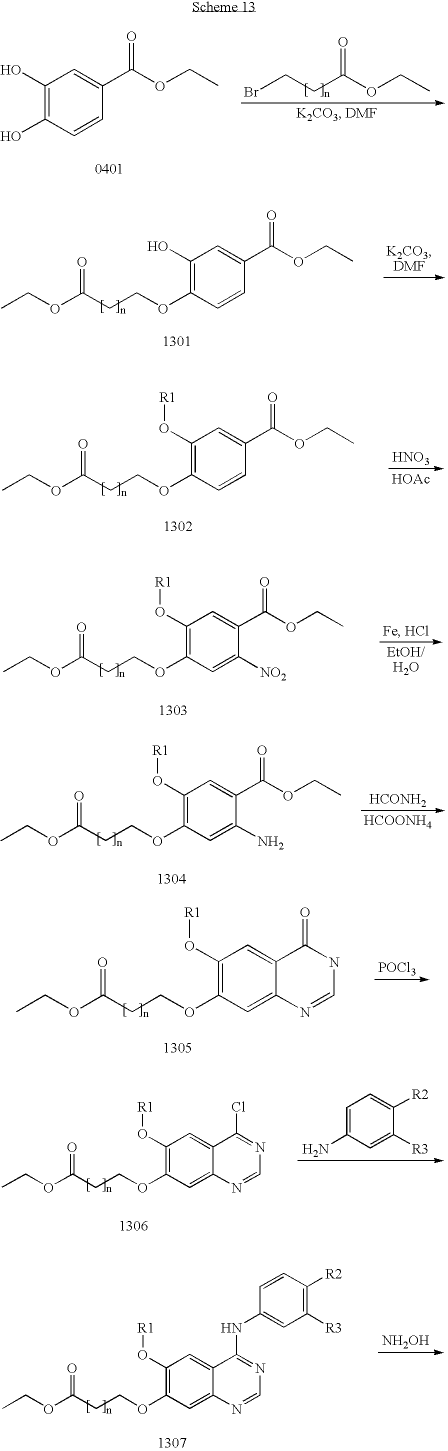

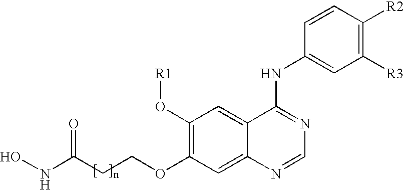

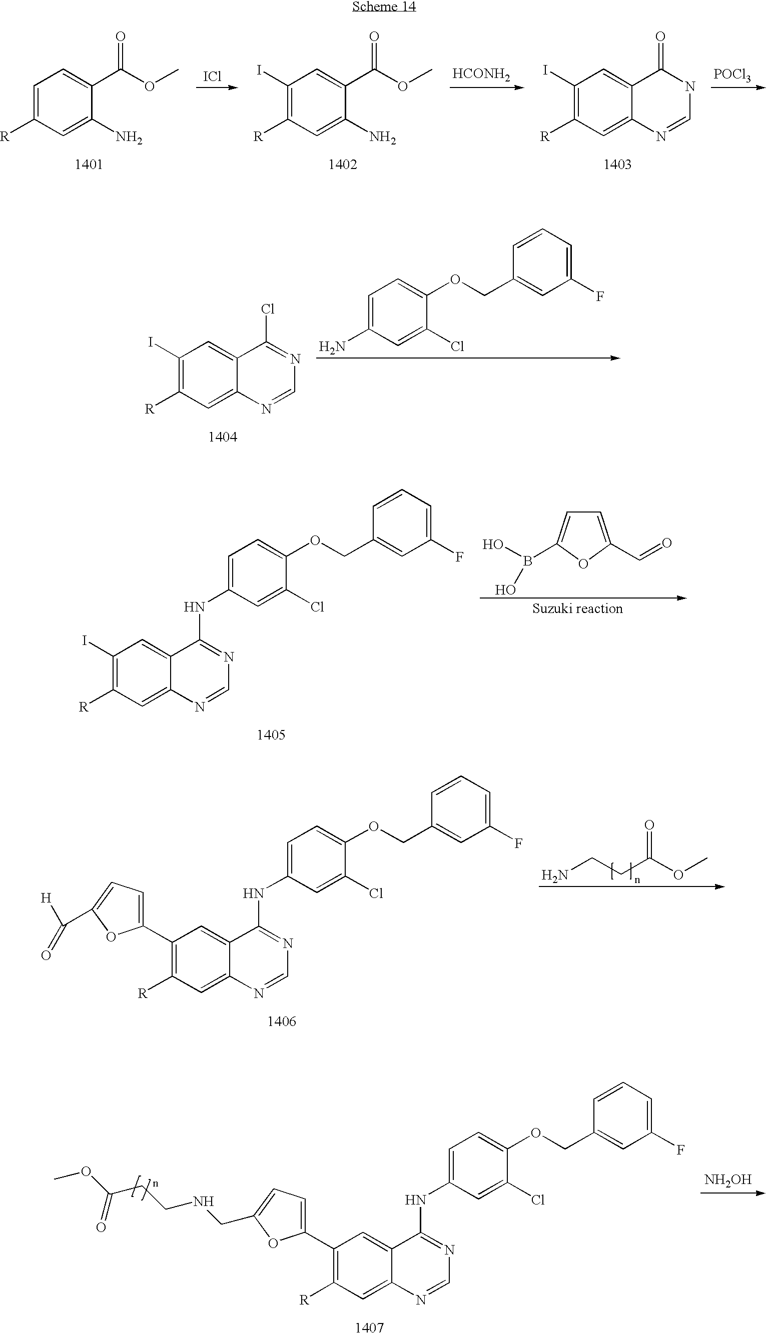

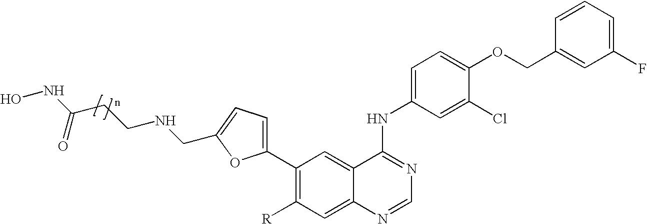

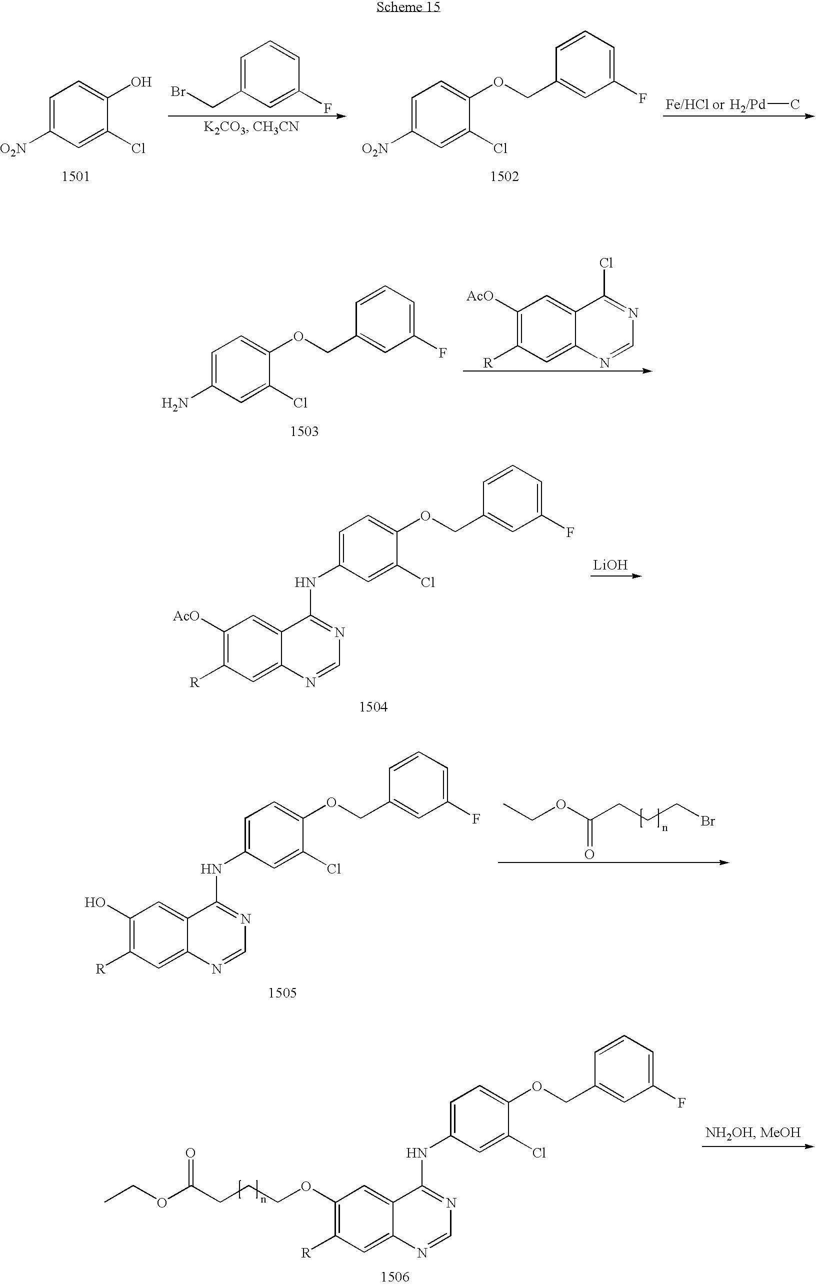

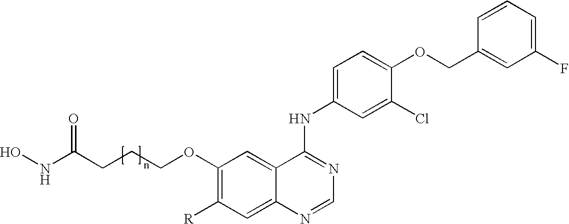

### Example 1

### Preparation of 2-(4-(3-Chloro-4-fluorophenylamino)-7-methoxyquinazolin-6-yloxy)-N-hydroxyacetamide (Compound 1)

### Step 1a. 6,7-Dimethoxyquinazolin-4(3H)-one (Compound 0102)

A mixture of methyl 2-amino-4,5-dimethoxybenzoic acid 0101 (2.1 g, 10 mmol), ammonium formate (0.63 g, 10 mmol) and formamide (7 ml) was stirred and heated to 190˜200° C. for 2 hours. Then the mixture was cooled to room temperature. The precipitate was isolated, washed with water and dried to provide the title compound 0102 as a brown solid (1.8 g, 84.7%): LCMS: m/z 207 [M+1]⁺; ¹H NMR (DMSO) δ 3.87 (s, 3H), 3.89 (s, 3H), 7.12 (s, 1H), 7.43 (s, 1H), 7.97 (s, 1H), 12.08 (bs, 1H).

### Step 1b. 6-Hydroxy-7-methoxyquinazolin-4(3H)-one (Compound 0103)

6,7-Dimethoxyquinazolin-4(3H)-one (0102) (10.3 g, 50 mmol) was added portionwise to stirred methanesulphonic acid (68 ml). L-Methionone (8.6 g, 57.5 mmol) was then added and resultant mixture was heated to 150˜160° C. for 5 hours. The mixture was cooled to room temperature and poured onto a mixture (250 ml) of ice and water. The mixture was neutralized by the addition of aqueous sodium hydroxide solution (40%). The precipitate was isolated, washed with water and dried to yield title compound 0103 as a grey solid (10 g, crude): LCMS: m/z 193 [M+1]⁺.

### Step 1c. 3,4-Dihydro-7-methoxy-4-oxoquinazolin-6-yl acetate (Compound 0104)

A mixture of 6-hydroxy-7-methoxyquinazolin-4(3H)-one (0103) (10 g crude), acetic anhydride (100 ml) and pyridine (8 ml) was stirred and heated to reflux for 3 hours. The mixture was cooled to room temperature and poured into a mixture (250 ml) of ice and water. The precipitate was isolated and dried to yield the title product 0104 as a grey solid (5.8 g, 50% two step overall yield): LCMS: m/z 235 [M+1]⁺; ¹H NMR (CDCl₃) δ 2.27 (s, 3H), 3.89 (s, 3H), 7.28 (s, 1H), 7.72 (s, 1H), 8.08 (d, 1H), 12.20 (bs, 1H).

### Step 1d. 4-Chloro-7-methoxyquinazolin-6-yl acetate (Compound 0105)

A mixture of 3,4-dihydro-7-methoxy-4-oxoquinazolin-6-yl acetate (0104) (2.0 g, 8.5 mmol) and phosphoryl trichloride (20 ml) was stirred and heated to reflux for 3 hours. When a clear solution was obtained, the excessive phosphoryl trichloride was removed under reduced pressure. The residue was dissolved in dichloromethane (50 ml) and the organic layer was washed with aqueous NaHCO₃ solution (20 ml×2) and brine (20 ml×1) and dried over MgSO₄, filtered and evaporated to give the title product 0105 as a yellow solid (1.4 g, 65%): LCMS: m/z 249 [M+1]⁺; ¹H NMR (CDCl₃) δ 2.40 (s, 3H), 4.03 (s, 3H), 7.44 (s, 1H), 7.90 (s, 1H), 8.95 (bs, 1H).

### Step 1e. 4-(3-Chloro-4-fluorophenylamino)-7-methoxyquinazolin-6-yl acetate hydrochloride (Compound 0108)

A mixture of 4-chloro-7-methoxyquinazolin-6-yl acetate (0105) (1.3 g, 5.1 mmol) and 3-chloro-4-fluorobenzenamine 0106 (1.5 g, 10.2 mmol) in isopropanol (45 ml) was stirred and heated to reflux for 3 hours. The mixture was cooled to room temperature and resulting precipitate was isolated. The solid was then dried to give the title compound 0108 as a light yellow solid (1.6 g, 79%): LCMS: m/z 362 [M+1]⁺; ¹H NMR (DMSO) δ 2.36 (s, 3H), 3.98 (s, 3H), 7.49 (s, 1H), 7.52 (d, 1H), 7.72 (m, 1H), 8.02 (dd, 1H), 8.71 (s, 1H), 8.91 (s, 1H), 11.4 (bs, 1H).

### Step 1f. 4-(3-Chloro-4-fluorophenylamino)-7-methoxyquinazolin-6-ol (Compound 0109)

A mixture of compound (0107) (1.41 g, 3.5 mmol), LiOH H₂O (0.5 g, 11.7 mmol) in methanol (100 ml) and H₂O (100 ml) was stirred at room temperature for 0.5 hour. The mixture was neutralized by addition of dilution acetic acid. The precipitate was isolated and dried to give the title compound 0109 as a grey solid (1.06 g, 94%): LCMS: m/z 320 [M+1]⁺; ¹H NMR (DMSO) δ 3.99 (s, 3H), 7.20 (s, 1H), 7.38 (t, 1H), 7.75 (s, 1H), 7.81 (m, 1H), 8.20 (m, 1H), 8.46 (s, 1H), 9.46 (s, 1H), 9.68 (s, 1H).

### Step 1g. Ethyl 2-(4-(3-chloro-4-fluorophenylamino)-7-methoxyquinazolin-6-yloxy)acetate (Compound 0110-1)

A mixture of compound 0109 (300 mg, 0.94 mmol) and Ethyl 2-bromoacetate (163 mg, 0.98 mmol) and potassium carbonate (323 mg, 2.35 mmol) in N,N-dimethylformamide (6 ml) was stirred and heated to 40° for 30 minutes. The reaction process was monitored by TLC. The mixture was filtrated. The filtration was concentrated under reduce pressure. The residues was wash with diethyl ether and dried to give the title compound 0110-1 as a yellow solid (280 mg, 74%): LCMS: m/z 406 [M+1]⁺; ¹H NMR (DMSO) δ 1.23 (t, 3H), 3.96 (s, 3H), 4.20 (q, 2H), 4.95 (s, 2H), 7.24 (s, 1H), 7.44 (t, 1H), 7.75 (m, 1H), 7.82 (s, 1H), 8.10 (dd, 1H), 8.51 (s, 1H), 9.54 (s, 1H).

### Step 1h. 2-(4-(3-Chloro-4-fluorophenylamino)-7-methoxyquinazolin-6-yloxy)-N-hydroxyacetamide (Compound 1)

To a stirred solution of hydroxyamine hydrochloride (4.67 g, 67 mmol) in methanol (24 ml) at 0° C. was added a solution of potassium hydroxide (5.61 g, 100 mmol) in methanol (14 ml). After addition, the mixture was stirred for 30 minutes at 0° C., and was allowed to stand at low temperature. The resulting precipitate was isolated, and the solution was prepared to give free hydroxyamine.

The above freshly prepared hydroxyamine solution (1.4 ml, 2.4 mmol) was placed in 5 ml flask. Compound 0110-1 (250 mg, 0.6 mmol) was added to this solution and stirred at 0° C. for 10 minutes, and raise to room temperature. The reaction process was monitored by TLC. The mixture was neutralized with acetic acid. The mixture was concentrated under reduce pressure. The residue was purified by preparation HPLC. To give the title compound 1 as a grey solid (50 mg, 21%): LCMS: m/z 393 [M+1]⁺; ¹H NMR (DMSO) δ 3.96 (s, 3H), 4.62 (s, 2H), 7.24 (s, 1H), 7.45 (t, 1H), 7.78 (m, 1H), 7.86 (s, 1H), 8.10 (dd, 1H), 8.52 (s, 1H), 9.07 (s, 1H), 9.57 (s, 1H), 10.80 (s, 1H).

### Example 2

### Preparation of 4-(4-(3-Chloro-4-fluorophenylamino)-7-methoxyquinazolin-6-yloxy)-N-hydroxybutanamide (Compound 3)

### Step 2a. Ethyl 4-(4-(3-chloro-4-fluorophenylamino)-7-methoxyquinazolin-6-yloxy)butanoate (Compound 0110-3)

The title compound 0110-3 was prepared as a yellow solid (220 mg, 80.5%) from compound 0109 from step 1f (200 mg, 0.63 mmol) and ethyl 4-bromobutyrate (135 mg, 0.69 mmol) using a procedure similar to that described for compound 0110-1 (example 1): LCMS: m/z 434 [M+1]⁺; ¹H NMR (CDCl₃) δ 1.36 (t, 3H), 2.23 (m, 2H), 2.57 (t, 2H), 4.03 (s, 3H), 4.32 (m, 4H), 7.15 (t, 1H), 7.25 (m, 1H), 7.87 (s, 1H), 8.00 (m, 2H), 8.15 (bs, 1H), 8.57 (s, 1H).

### Step 2b. 4-(4-(3-Chloro-4-fluorophenylamino)-7-methoxyquinazolin-6-yloxy)-N-hydroxybutanamide (Compound 3)

The title compound 3 was prepare as a grey solid (25 mg, 12%) from compound 0110-3 (200 mg, 0.23 mmol) using a procedure similar to that described for compound 1 (Example 1): LCMS: m/z 421 [M+1]⁺; ¹H NMR (DMSO): δ 2.06 (m, 2H), 2.22 (t, 2H), 3.95 (s, 3H), 4.15 (t, 2H), 7.21 (s, 1H), 7.43 (t, 1H), 7.83 (s, 2H), 8.14 (dd, 1H), 8.51 (s, 1H), 8.75 (s, 1H), 9.56 (s, 1H), 10.50 (s, 1H).

### Example 3

### Preparation of 7-(4-(3-Chloro-4-fluorophenylamino)-7-methoxyquinazolin-6-yloxy)-N-hydroxyhexanamide (Compound 5)

### Step 3a. Ethyl 6-(4-(3-chloro-4-fluorophenylamino)-7-methoxyquinazolin-6-yloxy)hexanoate (Compound 0110-5)

The title compound 0110-5 was prepared as a yellow solid (510 mg, 68%) from compound 0109 from step 1f (510 mg, 1.6 mmol) and ethyl 6-bromohexanoate (430 mg, 1.9 mmol) using a procedure similar to that described for compound 0110-1 (Example 1): LCMS: m/z 462 [M+1]⁺; ¹H NMR (CDCl₃): δ 1.24 (t, 3H), 1.55 (m, 2H), 1.74 (m, 2H), 1.91 (m, 2H), 2.38 (m, 2H), 3.97 (s, 3H), 4.13 (m, 4H), 7.15 (t, 1H), 7.25 (m, 2H), 7.60 (m, 1H), 7.86 (m, 1H), 7.91 (dd, 1H), 8.61 (s, 1H).

### Step 3b. 7-(4-(3-Chloro-4-fluorophenylamino)-7-methoxyquinazolin-6-yloxy)-N-hydroxyhexanamide (Compound 5)

The title compound 5 was prepared as a grey solid (100 mg, 34%) form compound 0110-5 (305 mg, 0.66 mmol) using a procedure similar to that described for compound 1 (Example 1): m.p. 206.6˜207.1° C. (dec); LCMS: m/z 449 [M+1]⁺; ¹H NMR (DMSO) δ 1.44 (m, 2H), 1.64 (m, 2H), 1.82 (m, 2H), 1.99 (t, 2H), 3.93 (s, 3H), 4.12 (t, 2H), 7.19 (s, 1H), 7.43 (t, 1H), 7.79 (m, 2H), 8.12 (dd, 1H), 8.49 (s, 1H), 8.68 (s, 1H), 9.53 (s, 1H), 10.37 (s, 1H).

### Example 4

### Preparation of 7-(4-(3-Chloro-4-fluorophenylamino)-7-methoxyquinazolin-6-yloxy)-N-hydroxyheptanamide (Compound 6)

### Step 4a. Ethyl 7-(4-(3-chloro-4-fluorophenylamino)-7-methoxyquinazolin-6-yloxy)heptanoate (Compound 0110-6)

The title compound 0110-6 was prepared as a yellow solid (390 mg, 53%) from compound 0109 from step 1f (512 mg, 1.6 mmol) and ethyl 7-bromoheptanoate (438 mg, 1.8 mmol) using a procedure similar to that described for compound 0110-1 (Example 1): LCMS: m/z 476 [M+1]⁺; ¹H NMR (CDCl₃) δ 1.24 (t, 3H), 1.43 (m, 4H), 1.66 (m, 2H), 1.88 (m, 2H), 2.32 (t, 2H), 3.97 (s, 3H), 4.07 (t, 2H), 4.12 (q, 2H), 7.15 (t, 1H), 7.23 (t, 2H), 7.66 (m, 1H), 7.75 (m, 1H), 7.87 (dd, 1H), 8.65 (s, 1H).

### Step 4b. 7-(4-(3-Chloro-4-fluorophenylamino)-7-methoxyquinazolin-6-yloxy)-N-hydroxyheptanamide (Compound 6)

The title compound 6 was prepared as a grey solid (80 mg, 25%) from compound 0110-6 (323 mg, 0.68 mmol) using a procedure similar to that described for compound 1 (Example 1): m.p. 180.8˜182.3° C. (dec); LCMS: m/z 463 [M+1]⁺; ¹H NMR (DMSO) δ 1.34 (m, 2H), 1.50 (m, 4H), 1.81 (m, 2H), 1.96 (t, 2H), 3.92 (s, 3H), 4.11 (t, 2H), 7.18 (s, 1H), 7.43 (t, 1H), 7.78 (m, 2H), 8.12 (dd, 1H), 8.48 (s, 1H), 8.64 (s, 1H), 9.50 (s, 1H), 10.33 (s, 1H).

### Example 5

### Preparation of 2-(4-(3-ethynylphenylamino)-7-methoxyquinazolin-6-yloxy)-N-hydroxyacetamide (Compound 7)

### Step 5a. 4-(3-Ethynylphenylamino)-7-methoxyquinazolin-6-yl acetate Hydrochloride (Compound 0111)

A mixture of 4-chloro-7-methoxyquinazolin-6-yl acetate (0105) (2.6 g, 10.2 mmol) and 3-ethynylbenzenamine (0107) (2.4 g, 20.5 mmol) in isopropanol (100 ml) was stirred and heated to reflux for 3 hours. The mixture was cooled to room temperature. The precipitate was isolated and dried to give the title compound 0111 as a yellow solid (2.6 g, 68%): LCMS: m/z 334 [M+1]⁺; ¹H NMR (DMSO) δ 2.39 (s, 3H), 3.17 (s, 1H), 3.98 (s, 3H), 7.35 (m, 1H), 7.40 (s, 1H), 7.47 (m, 1H), 7.72 (m, 1H), 7.90 (s, 1H), 8.57 (s, 1H), 8.87 (s, 1H), 10.99 (bs, 1H).

### Step 5b. 4-(3-Ethynylphenylamino)-7-methoxyquinazolin-6-ol (Compound 0112)

A mixture of compound 0111 (2.0 g, 5.4 mmol) and LiOH H₂O (0.75 g, 17.9 mmol) in methanol (100 ml) and H₂O (100 ml) was stirred at room temperature for 0.5 hour. The mixture was neutralized by addition of dilution acetic acid. The precipitate was isolated and dried to give the title compound 0112 as a grey solid (1.52 g, 96%): LCMS: m/z 292 [M+1]⁺; ¹H NMR (DMSO) δ 3.17 (s, 1H), 3.98 (s, 3H), 7.18 (d, 1H), 7.21 (s, 1H), 7.37 (t, 1H), 7.80 (s, 1H), 7.90 (d, 1H), 8.04 (m, 1H), 8.47 (s, 1H), 9.41 (s, 1H), 9.68 (bs, 1H).

### Step 5c. Ethyl 2-(4-(3-ethynylphenylamino)-7-methoxyquinazolin-6-yloxy)acetate (Compound 0113-7)

The title compound 0113-7 was prepared as a yellow solid (450 mg, 69%) from compound 0112 (500 mg, 1.72 mmol) and ethyl 2-bromoacetate (300 mg, 1.8 mmol) using a procedure similar to that described for compound 0110-1 (Example 1): LCMS: m/z 378 [M+1]⁺; ¹H NMR (DMSO) δ 1.22 (t, 3H), 3.97 (s, 3H), 4.21 (q, 2H), 4.97 (t, 2H), 7.22 (d, 1H), 7.24 (s, 1H), 7.42 (t, 1H), 7.84 (m, 2H), 7.86 (d, 1H), 7.96 (s, 1H), 8.51 (s, 1H).

### Step 5d. 2-(4-(3-Ethynylphenylamino)-7-methoxyquinazolin-6-yloxy)-N-hydroxyacetamide (Compound 7)

The title compound 7 was prepared as a grey solid (100 mg, 23%) from compound 0113-7 (448 mg, 1.2 mmol) using a procedure similar to that described for compound 1 (Example 1): LCMS: m/z 365 [M+1]⁺; ¹H NMR (DMSO) δ 4.00 (s, 3H), 4.26 (s, 1H), 4.65 (s, 2H), 7.27 (s, 1H), 7.37 (d, 1H), 7.49 (t, 1H), 7.73 (d, 1H), 7.85 (s, 1H), 8.03 (s, 1H), 8.78 (s, 1H), 9.17 (bs, 1H), 10.60 (s, 1H), 10.85 (s, 1H).

### Example 6

### Preparation of 4-(4-(3-ethynylphenylamino)-7-methoxyquinazolin-6-yloxy)-N-hydroxybutanamide (Compound 9)

### Step 6a. Ethyl 4-(4-(3-ethynylphenylamino)-7-methoxyquinazolin-6-yloxy)butanoate (Compound 0113-9)

The title compound 0113-9 was prepared as a yellow solid (438 mg, 59%) from compound 0112 (500 mg, 1.72 mmol) and ethyl 4-bromobutyrate (349 mg, 1.8 mmol) using a procedure similar to that described for compound 0110-1 (Example 1): LCMS: m/z 406 [M+1]⁺; ¹H NMR (CDCl₃) δ 1.37 (t, 3H), 2.34 (m, 2H), 2.56 (t, 2H), 3.07 (s, 1H), 4.03 (s, 3H), 4.32 (m, 4H), 7.21 (m, 1H), 7.25 (s, 1H), 7.36 (t, 1H), 7.94 (s, 1H), 7.97 (m, 1H), 8.20 (s, 1H), 8.28 (m, 1H), 8.70 (s, 1H).

### Step 6b. 4-(4-(3-Ethynylphenylamino)-7-methoxyquinazolin-6-yloxy)-N-hydroxybutanamide (Compound 9)

The title compound 9 was prepared as a grey solid (60 mg, 31%) from compound 0113-9 (200 mg, 0.49 mmol) using a procedure similar to that described for compound 1 (Example 1): LCMS: m/z 393 [M+1]⁺; ¹H NMR (DMSO) δ 2.06 (m, 2H), 2.22 (t, 2H), 3.30 (s, 1H), 3.95 (s, 3H), 4.16 (t, 2H), 7.19 (m, 2H), 7.40 (t, 1H), 7.85 (s, 1H), 7.91 (d, 1H), 8.02 (s, 1H), 8.51 (s, 1H), 8.74 (s, 1H), 9.49 (s, 1H), 10.49 (s, 1H).

### Example 7

### Preparation of 6-(4-(3-ethynylphenylamino)-7-methoxyquinazolin-6-yloxy)-N-hydroxyhexanamide (Compound 11)

### Step 7a. Ethyl 6-(4-(3-ethynylphenylamino)-7-methoxyquinazolin-6-yloxy)hexanoate (Compound 0113-11)

The title compound 0113-11 was prepared as yellow solid (543 mg, 73%) from compound 0112 from step 5b (500 mg, 1.72 mmol) and ethyl 6-bromohexanoate (401 mg, 1.8 mmol) using a procedure similar to that described for compound 0110-1 (Example 1): LCMS: m/z 434 [M+1]⁺; ¹H NMR (CDCl₃) δ 1.24 (t, 3H), 1.53 (m, 2H), 1.72 (m, 2H), 1.90 (m, 2H), 2.37 (t, 3H), 3.08 (s, 1H), 3.97 (s, 3H), 4.10 (m, 4H), 7.19 (s, 1H), 7.25 (m, 2H), 7.34 (t, 1H), 7.67 (s, 1H), 7.78 (m, 1H), 7.84 (m, 1H), 8.67 (s, 1H).

### Step 7b. 6-(4-(3-Ethynylphenylamino)-7-methoxyquinazolin-6-yloxy)-N-hydroxyhexanamide (Compound 11)

The title compound 11 was prepared as a grey solid (110 mg, 41%) from compound 0113-11 (275 mg, 0.63 mmol) using a procedure similar to that described for compound 1 (Example 1): m.p. 193.4˜195.8° C. (dec); LCMS: m/z 421 [M+1]⁺; ¹H NMR (DMSO) δ 1.44 (m, 2H), 1.60 (m, 2H), 1.84 (m, 2H), 1.99 (t, 2H), 3.93 (s, 3H), 4.13 (t, 2H), 4.19 (s, 1H), 7.19 (m, 2H), 7.40 (t, 1H), 7.81 (s, 1H), 7.88 (d, 1H), 7.98 (s, 1H), 8.49 (s, 1H), 8.68 (s, 1H), 9.47 (s, 1H), 10.39 (s, 1H).

### Example 8

### Preparation of 7-(4-(3-ethynylphenylamino)-7-methoxyquinazolin-6-yloxy)-N-hydroxyheptanamide (Compound 12)

### Step 8a. Ethyl 6-(4-(3-ethynylphenylamino)-7-methoxyquinazolin-6-yloxy)heptanoate (Compound 0113-12)

The title compound 0113-12 was prepared as a yellow solid (305 mg, 84%) from compound 0112 from step 5b (247 mg, 0.85 mmol) and ethyl 7-bromohepanoate (211 mg, 0.89 mmol) using a procedure similar to that described for compound 0110-1 (Example 1): LCMS: 448 [M+1]⁺; ¹H NMR (CDCl₃): δ1.15 (t, J=7.5 Hz, 3H), 1.33-1.60 (m, 6H), 1.81 (m, 2H), 2.28 (t, J=7.5 Hz, 2H), 3.92 (s, 3H), 4.03 (q, J=7.2 Hz, 2H), 4.12 (t, J=6.6 Hz, 2H), 4.18 (s, 1H), 7.19 (m, 2H), 7.39 (t, J=7.8 Hz, 1H), 7.80 (s, 1H), 7.89 (d, J=8.1 Hz, 1H), 7.97 (s, 1H), 8.48 (s, 1H), 9.44 (s, 1H).

### Step 8b. 7-(4-(3-Ethynylphenylamino)-7-methoxyquinazolin-6-yloxy)-N-hydroxyheptanamide (Compound 12)

The title compound 12 was prepared as a grey solid (100 mg, 41%) from compound 0113-12 (250 mg, 0.56 mmol) using a procedure similar to that described for compound 1 (Example 1): m.p. 171.8˜177.2° C. (dec); LCMS: 435 [M+1]⁺; ¹H NMR (DMSO-d₆): δ1.36 (m, 2H), 1.52 (m, 4H), 1.83 (m, 2H), 1.97 (m, 2H), 3.94 (s, 3H), 4.14 (t, J=6.3 Hz, 2H), 4.20 (s, 1H), 7.21 (m, 2H), 7.41 (t, J=8.1 Hz, 1H), 7.83 (s, 1H), 7.90 (d, J=8.1 Hz, 1H), 8.00 (s, 1H), 8.50 (s, 1H), 8.66 (s, 1H), 9.48 (s, 1H), 10.35 (s, 1H).

### Example 8

### (METHOD 2): Preparation of 7-(4-(3-ethynylphenylamino)-7-methoxyquinazolin-6-yloxy)-N-hydroxyheptanamide (Compound 12)

### Step 8a′. Ethyl 3-hydroxy-4-methoxybenzoate (Compound 0402-12)

To a solution of ethyl 3,4-dihydroxybenzoate 0401 (12.52 g, 68.7 mmol) in DMF (50 mL) was added potassium carbonate (9.48 g, 68.7 mmol). After the mixture was stirred for 15 minutes, a solution of iodomethane (9.755 g, 68.7 mmol) in DMF (10 mL) was added dropwise. The reaction mixture was stirred at 20° C. for 24 hours. After reaction the mixture was filtered, and the filtrate was concentrated. The residue was dissolved in dichloromethane and washed with brine. The organic phase was dried over sodium sulfate, filtered and concentrated in vacuo to give crude product. The crude product was purified by column chromatography to give the title compound 0402-12 as a white solid (7.1 g, 53%): LCMS: 197 [M+1]⁺, ¹H NMR (DMSO-d₆): δ 1.29 (t, J=6.6 Hz, 3H), 3.83 (s, 3H), 4.25 (q, J=6.6 Hz, 2H), 7.00 (d, J=8.4 Hz, 1H), 7.38 (d, J=1.8 Hz, 1H), 7.43 (dd, J=8.4 Hz, 2.1 Hz, 1H), 9.36 (s, 1H).

### Step 8b′. Ethyl 3-(7-ethoxy-7-oxoheptyloxy)-4-methoxybenzoate (Compound 0403-12)

A mixture of compound 0402-12 (6.34 g, 32.3 mmol), ethyl 7-bromoheptanoate (7.66 g, 32.3 mmol) and potassium carbonate (13.38 g, 96.9 mmol) in DMF (80 mL) was stirred at 60° C. for 3 hours. After reaction the mixture was filtrated. The filtrate was concentrated in vacuo and the residue was dissolved in dichloromethane and washed with brine twice. The organic phase was dried over sodium sulfate, filtered and concentrated to give the title product 0403-12 as a white solid (9.87 g, 86.7%): LCMS: 353 [M+1]⁺, ¹H NMR (DMSO-d₆): δ1.17 (t, J=6.9 Hz, 3H), 1.31 (t, J=7.2 Hz, 3H) 1.39 (m, 4H), 1.54 (m, 2H), 1.72 (m, 2H), 2.29 (t, J=7.2 Hz, 2H), 3.83 (s, 3H), 3.98 (t, J=7.2 Hz, 2H), 4.06 (q, J=6.9 Hz, 2H), 4.29 (q, J=7.2 Hz, 2H), 7.06 (d, J=8.4 Hz, 1H), 7.42 (d, J=1.8 Hz, 1H), 7.57 (dd, J=8.4 Hz, 1.8 Hz, 1H).

### Step 8c′. Ethyl 5-(7-ethoxy-7-oxoheptyloxy)-4-methoxy-2-nitrobenzoate (Compound 0404-12)

Compound 0403-12 (9.87 g, 28.0 mmol) was dissolved in acetic acid (20 mL) and stirred at 20° C. Fuming nitric acid (17.66 g, 280.0 mmol) was added slowly dropwise. The mixture was stirred at 20° C. for 1 hour. After reaction the mixture was poured into ice-water and extracted with dichloromethane twice. The combined organic phase was washed with brine, aqueous NaHCO₃ solution and brine. The combined organic phase was dried over sodium sulfate, filtered and concentrated to give the title product 0404-12 as a yellow solid (10.75 g, 96.4%): LCMS: 398 [M+1]⁺, ¹H NMR (DMSO-d₆): δ1.17 (t, J=7.2 Hz, 3H), 1.27 (t, J=7.2 Hz, 3H), 1.38 (m, 4H), 1.53 (m, 2H), 1.74 (m, 2H), 2.29 (t, J=7.2 Hz, 2H), 3.91 (s, 3H), 4.03 (q, J=7.2 Hz, 2H), 4.08 (t, J=6.3 Hz, 2H), 4.30 (q, J=7.2 Hz, 2H), 7.29 (s, 1H), 7.63 (s, 1H).

### Step 8d′. Ethyl 2-amino-5-(7-ethoxy-7-oxoheptyloxy)-4-methoxybenzoate (Compound 0405-12)

A mixture of 0404-12 (10.75 g 27.0 mmol), ethanol (120 mL), water (40 mL) and hydrogen chloride (4 mL) was stirred to form a clear solution. The iron powder (15.16 g, 27.0 mmol) was added batchwise. The mixture was stirred at reflux for 30 min, and was then cooled to room temperature, adjusted pH to 8 with 10% sodium hydroxide solution, and filtered. The filtrate was concentrated to remove ethanol and extracted with dichloromethane twice. The combined organic phase was washed with brine and dried over sodium sulfate, filtered and concentrated to give the title product 0405-12 as a yellow solid (8.71 g, 87.8%): LCMS: 368 [M+1]⁺, ¹H NMR (DMSO-d₆): δ 1.17 (t, J=7.2 Hz, 3H), 1.28 (t, J=7.2 Hz, 3H), 1.37 (m, 4H), 1.53 (m, 2H), 1.66 (m, 2H), 2.29 (t, J=7.2 Hz, 2H), 3.74 (s, 3H), 3.78 (t, J=6.9 Hz, 2H), 4.06 (q, J=7.2 Hz, 2H), 4.22 (q, J=7.2 Hz, 2H), 6.35 (s, 1H), 6.44 (s, 2H), 7.15 (s, 1H).

### Step 8e′. Ethyl 7-(7-methoxy-4-oxo-3,4-dihydroquinazolin-6-yloxy)heptanoate (Compound 0406-12)

A mixture of compound 0405-12 (8.71 g, 23.7 mmol), ammonium formate (1.48 g, 23.7 mmol) and formamide (40 mL) was stirred at 180° C. for 3 hours. After reaction the mixture was cooled to room temperature. The formamide was removed under reduce pressure, and the residue was dissolved in dichloromethane and washed with brine. The organic phase was dried over sodium sulfate, filtered and concentrated to give the title product 0406-12 as a pale white solid (8.18 g, 99%): LCMS: 349 [M+1]⁺, ¹H NMR (DMSO-d₆): δ1.17 (t, J=6.9 Hz, 3H), 1.38 (m, 4H), 1.55 (m, 2H), 1.75 (m, 2H), 2.29 (t, J=7.2 Hz, 2H), 3.90 (s, 3H), 4.05 (m, 4H), 7.13 (s, 1H), 7.42 (s, 1H), 7.97 (d, J=3.6 Hz, 1H), 12.07 (s, 1H).

### Step 8f′. Ethyl 7-(4-chloro-7-methoxyquinazolin-6-yloxy)heptanoate (Compound 0407-12)

A mixture of product 0406-12 (8.18 g, 23.5 mmol) and phosphoryl trichloride (50 mL) was stirred at reflux for 4 hours. After reaction the excessive phosphoryl trichloride was removed under reduced pressure. The residue was dissolved in dichloromethane and washed with water, aqueous NaHCO₃ solution and brine. The organic phase was dried over sodium sulfate, filtered and concentrated to give the title product 0407-12 as a yellow solid (5.93 g, 69.7%): LCMS: 367 [M+1]⁺, ¹H NMR (DMSO-d₆): δ1.17 (t, J=6.9 Hz, 3H), 1.38 (m, 4H), 1.54 (m, 2H), 1.81 (m, 2H), 2.30 (t, J=7.2 Hz, 2H), 4.02 (s, 3H), 4.06 (q, J=6.9 Hz, 2H), 4.18 (t, J=6.3 Hz, 2H), 7.37 (s, 1H), 7.45 (s, 1H), 8.87 (s, 1H).

### Step 8g′. Ethyl 7-(4-(3-ethynylphenylamino)-7-methoxyquinazolin-6-yloxy)heptanoate (Compound 0408-12)

A mixture of product 0407-12 (5.93 g, 16.4 mmol) and 3-ethynylbenzenamine (1.92 g, 16.4 mmol) in isopropanol (80 mL) was stirred at reflux 4 hours. After reaction the mixture was cooled to room temperature and resulting precipitate was isolated, washed with isopropanol and ether, and dried to give the title compound 0408-12 as a yellow solid (4.93 g, 67.1%): LCMS: 448 [M+1]⁺, ¹H NMR (DMSO-d₆): δ1.16 (t, J=7.2 Hz, 3H), 1.36-1.59 (m, 6H), 1.80 (m, 2H), 2.29 (t, J=7.2 Hz, 2H), 3.93 (s, 3H), 4.04 (q, J=6.9 Hz, 2H), 4.13 (t, J=6.6 Hz, 2H), 4.19 (s, 1H), 7.20 (m, 2H), 7.39 (t, J=7.8 Hz, 1H), 7.81 (s, 1H), 7.89 (d, J=8.4 Hz, 1H), 7.97 (s, 1H), 8.48 (s, 1H), 9.45 (s, 1H).

### Step 8h′. 7-(4-(3-ethynylphenylamino)-7-methoxyquinazolin-6-yloxy)-N-hydroxyheptanamide (Compound 12)

The freshly prepared hydroxylamine solution (30 mL, 110 mmol) was placed in 50 mL flask. Compound 0408-12 (4.93 g, 11.0 mmol) was added to this solution and stirred at 25° C. for 24 hours. After reaction the mixture was neutralized with acetic acid, and the resulting precipitate was isolated, washed with water, and dried to give the title compound 12 as a white solid (3.99 g, 83.6%): mp 174.1˜177.2° C. LCMS: 435 [M+1]⁺, ¹H NMR (DMSO-d₆): δ1.36 (m, 2H), 1.52 (m, 4H), 1.83 (m, 2H), 1.98 (m, 2H), 3.94 (s, 3H), 4.14 (t, J=6.6 Hz, 2H), 4.20 (s, 1H), 7.21 (m, 2H), 7.41 (t, J=7.8 Hz, 1H), 7.80 (s, 1H), 7.90 (d, J=7.8 Hz, 1H), 8.00 (s, 1H), 8.50 (s, 1H), 8.66 (s, 1H), 9.48 (s, 1H), 10.35 (s, 1H).

### Example 9

### Preparation of 2-(4-(3-chloro-4-fluorophenylamino)quinazolin-6-yloxy)-N-hydroxyacetamide (Compound 13)

### Step 9a. 6-Hydroxyquinazolin-4(3H)-one (compound 0202)

To a solution of 2-amino-5-hydroxybenzoic acid 0201 (30.6 g, 0.2 mol) in formamide was stirred and heated to 190° C. for 0.5 h. The mixture was allowed to cool to room temperature. The precipitate was isolated, washed with ether and dried to obtain title compound 0202 (32 g, brown solid, yield: 99%): LC-MS m/z 163 [M+1]; ¹H NMR (DMSO) δ7.25 (dd, 1H), 7.40 (d, 1H), 7.46 (d, 1H), 7.88 (s, 1H).

### Step 9b. 3,4-Dihydro-4-oxoquinazolin-6-yl acetate (Compound 0203)

A mixture of compound 0202 (30.0 g, 0.185 mol) and pyridine (35 ml) in acetic anhydride (275 ml) was stirred and heated at 100° C. for 2 hours. The reaction was poured into a mixture of ice and water (500 ml). The precipitate was isolated, washed with water and dried to obtain the title compound 0203 (24 g, pale white solid, yield: 61%): LC-MS m/z 205 [M+1]; 1H-NMR (DMSO) δ 2.32 (s, 3H), 7.50 (dd, 1H), 7.80 (d, 1H), 7.98 (s, 1H), 8.02 (s, 1H).

### Step 9c. 4-Chloroquinazolin-6-yl acetate (Compound 0204)

A mixture of compound 0203 (20.0 g, 0.1 mol) in POCl₃ (150 ml) was stirred and heated to reflux for 2 hours. The reaction was evaporated and the residue was partitioned between ethyl acetate and a saturated aqueous NaHCO₃ solution. The organic phase was washed with water, dried over Na₂SO₄ and evaporated. The mixture was purified by column chromatography (silica gel, elution: 1:2=ethyl acetate/petroleum) to obtained the title compound 0204 (7.5 g, white solid, yield: 35%): LC-MS m/z 223 [M+1]; ¹H-NMR (CDCl3) δ2.40 (s, 3H), 7.74 (dd, 1H), 8.00 (d, 1H), 8.09 (d, 1H), 9.05 (s, 1H).

### Step 9d. 4-(3-Chloro-4-fluorophenylamino)quinazolin-6-yl acetate (Compound 0207)

A mixture of 0204 (1.0 g, 4.5 mmol) and 3-chloro-4-fluorobenzenamine 0205 (0.7 g, 5.0 mmol) in isopropanol (45 ml) was stirred and heated at 90° C. for 1 hours. The reaction was cooled to room temperature and the precipitate was isolated. The solid was washed in turn with isopropanol and methanol, dried to provide the title compound 0207 (1.3 g, pale yellow solid, yield: 87%): LC-MS m/z 332 [M+1]; 1H-NMR (DMSO) δ2.37 (s, 3H), 7.54 (t, 1H), 7.75 (m, 1H), 7.94 (dd, 1H), 7.99 (s, 1H), 8.02 (m, 1H), 8.64 (s, 1H), 8.95 (s, 1H).

### Step 9e. 4-(3-Chloro-4-fluorophenylamino)quinazolin-6-ol (Compound 0209)

A mixture of 0207 (0.8 g, 2.6 mmol) and lithium hydroxide monohydrate (0.13 g, 3.2 mmol) in methanol (10 ml)/water (15 ml) was stirred at room temperature for 1 hour. The pH was adjusted to 4 with acetic acid and filtered. The collected yellow solid was washed by water and dried to obtained title compound 0209 (0.6 g, yellow solid, yield: 88%): LC-MS m/z 290 [M+1]; ¹H-NMR (DMSO) δ7.42 (s, 1H), 7.45 (m, 1H), 7.70 (d, 1H), 7.76 (s, 1H), 7.86 (m, 1H), 8.24 (q, 1H), 8.48 (s, 1H), 9.61 (s, 1H).

### Step 9f. Ethyl 2-(4-(3-chloro-4-fluorophenylamino)quinazolin-6-yloxy)acetate (Compound 0210-13)

A mixture of 0209 (0.2 g, 0.77 mmol), ethyl 3-bromopropanoate (0.14 g, 0.85 mmol) and K₂CO₃ (0.8 g, 5.8 mmol) in DMF (15 ml) was stirred and heated to 80° C. for 2 hours. The reaction was filtered and the filtrate was evaporated. The resulting solid was washed with ether to obtain the title compound 0210-13 (0.2 g, yellow solid, yield: 75%): mp 161-163° C.; LC-MS m/z 376 [M+1]; 1H-NMR (DMSO) δ1.20 (t, 3H), 4.20 (q, 2H), 4.96 (s, 2H), 7.45 (t, 1H), 7.55 (dd, 1H), 7.78 (m, 2H), 7.94 (d, 1H), 8.16 (dd, 1H), 8.54 (s, 1H), 9.69 (s. 1H).

### Step 9g. 2-(4-(3-Chloro-4-fluorophenylamino)quinazolin-6-yloxy)-N-hydroxyacetamide (Compound 13)

To a stirred solution of hydroxyamine hydrochloride (4.67 g, 67 mmol) in methanol (24 ml) at 0° C. was added a solution of potassium hydroxide (5.61 g, 100 mmol) in methanol (14 ml). After addition, the mixture was stirred for 30 minutes at 0° C., and was allowed to stand at low temperature. The resulting precipitate was isolated, and the solution was prepared to give free hydroxyamine.

Take above solution (1.4 ml, 2.4 mmol) into 5 ml flask. Compound 0210-13 (0.1 g, 0.29 mmol) was added into this solution and stirred at 0° C. for 10 minutes, and then allowed to warm to room temperature. The reaction process was monitored by TLC. The mixture was adjusted pH to 6 with acetic acid and then concentrated under reduce pressure. The residue was purified by preparation HPLC eluted by methanol/water. The band containing the product was collected. The solvent was evaporated to obtain title compound 13 (30 mg, yellow solid, yield: 29%): LC-MS m/z 363 [M+1]; 1H-NMR (DMSO) δ4.64 (s, 2H), 7.46 (t, 1H), 7.58 (d, 1H), 7.79 (d, 2H), 7.7 (s, 1H), 8.11 (s, 1H), 8.52 (s, 1H), 9.02 (s, 1H), 9.67 (s, 1H), 10.96 (s, 1H).

### Example 10

### Preparation of 4-(4-(3-chloro-4-fluorophenylamino)quinazolin-6-yloxy)-N-Hydroxybutanamide (Compound 15)

The title compound 15 was prepared (20 mg) from compound 0209 from step 9e and ethyl 4-bromobutanoate using a procedure similar to that described for compound 13 (Example 9): mp 128-132° C.; LC-MS m/z 391 [M+1]; ¹H-NMR (DMSO+D2O) δ2.05 (m. 2H), 2.24 (t, 2H), 4.21 (t, 2H) 7.46 (t, 1H), 7.54 (dd, 1H), 7.65 (m, 1H), 7.76 (d, 1H), 7.82 (m 1H), 7.99 (m, 1H), 8.43 (s, 1H).

### Example 11

### Preparation of 6-(4-(3-chloro-4-fluorophenylamino)quinazolin-6-yloxy)-N-hydroxy hexanamide (Compound 17)

### Step 11a. Ethyl 6-(4-(3-chloro-4-fluorophenylamino)quinazolin-6-yloxy)hexanoate (Compound 0210-17)

The title compound 0210-17 (0.2 g) was prepared from compound 0209 4-(3-chloro-4-fluorophenylamino)quinazolin-6-ol and ethyl 6-bromohexanoate using a procedure similar to that described for compound 0210-13 (Example 9): LC-MS m/z 433 [M+1], ¹H-NMR (DMSO) δ1.13 (t, 3H), 1.45 (m, 2H), 1.60 (m, 2H) 1.76 (m, 2H), 2.30 (t, 2H), 4.05 (q, 2H), 4.11 (t, 2H), 7.41 (d, 1H), 7.45 (dd, 1H), 7.68 (d, 1H), 7.80 (m, 1H), 7.86 (m, 1H), 8.13 (dd, 1H), 8.48 (s, 1H).

### Step 11b. 6-(4-(3-Chloro-4-fluorophenylamino)quinazolin-6-yloxy)-N-hydroxyhexanamide (Compound 17)

The title compound 17 (30 mg) was prepared from compound 0210-17 using a procedure similar to that described for compound 13 (Example 9): LC-MS [M+1] 419 ¹H-NMR (DMSO) δ1.28 (m, 2H), 1.60 (m, 2H) 1.73 (m, 2H), 2.05 (t, 2H), 4.17 (t, 2H), 7.25 (d, 1H), 7.47 (t, 1H), 7.55 (dd, 1H) 7.76 (d, 1H) 7.73 (m, 1H), 8.05 (m, 1H), 8.48 (s, 1H).

### Example 12

### Preparation of 7-(4-(3-chloro-4-fluorophenylamino)quinazolin-6-yloxy)-N-hydroxyheptanamide (Compound 18)

### Step 12a. Ethyl 7-(4-(3-chloro-4-fluorophenylamino)quinazolin-6-yloxy)heptanoate (Compound 0210-18)

The title compound 0210-18 (0.2 g) was prepared from compound 2-6 4-(3-chloro-4-fluorophenylamino)quinazolin-6-ol (0209) of step 9e and ethyl 7-bromoheptanoate using a procedure similar to that described for compound 0210-13 (Example 9): LC-MS m/z 420 [M+1], ¹H-NMR (DMSO) δ1.13 (t, 3H), 1.36 (m, 2H), 1.46 (m, 2H), 1.54 (m, 2H) 1.78 (m, 2H), 2.27 (t, 2H), 4.05 (q, 2H), 4.11 (t, 2H), 7.41 (d, 1H), 7.47 (dd, 1H), 7.70 (d, 1H), 7.81 (m, 1H), 7.84 (m, 1H), 8.13 (dd, 1H), 8.50 (s, 1H).

### Step 12b. 7-(4-(3-Chloro-4-fluorophenylamino)quinazolin-6-yloxy)-N-hydroxyheptanamide (Compound 18)

The title compound 18 (20 mg) was prepared from compound ethyl 7-(4-(3-chloro-4-fluorophenylamino)quinazolin-6-yloxy-heptanoate (0210-18) using a procedure similar to that described for compound 13 (Example 9): LC-MS m/z 433 [M+1], mp 145-149° C., ¹H-NMR (DMSO) δ1.32 (m, 2H), 1.47 (m, 4H) 1.88 (m, 2H), 1.94 (t, 2H), 4.12 (t, 2H), 7.43 (t, 1H), 7.51 (dd, 1H), 7.71 (d, 1H) 7.80 (m, 1H) 7.86 (d, 1H), 8.15 (dd, 1H), 8.51 (s, 1H).

### Example 13

### Preparation of 2-(4-(3-ethynylphenylamino)quinazolin-6-yloxy)-N-hydroxyacetamide (Compound 19)

### Step 13a. 4-(3-Ethynylphenylamino)quinazolin-6-yl acetate (Compound 0208)

The title compound 0208 (0.8 g, yield: 73%) was prepared from 4-chloroquinazolin-6-yl acetate 0204 and 3-ethynylbenzenamine 0206 using a procedure similar to that described for compound 0207 (Example 9): LC-MS m/z 304 [M+1], ¹H-NMR (DMSO) δ2.36 (s, 3H), 4.26 (s, 1H), 7.43 (d, 1H), 7.53 (t, 1H), 7.77 (d, 1H), 7.95 (m, 2H), 8.02 (d, 1H), 8.71 (s, 1H), 8.96 (s, 1H).

### Step 13b. 4-(3-Ethynylphenylamino)quinazolin-6-ol (Compound 0211)

The title compound 0211 (0.6 g, yield: 88%) was prepared using a procedure similar to that described for compound 0209 (Example 9): LC-MS m/z 262 [M+1], 1H-NMR (DMSO) δ4.17 (s, 1H), 7.19 (d, 1H), 7.36 (t, 1H), 7.43 (dd, 1H, 7.65 (d, 1H), 0.82 (d, 1H), 95 (d, 1H), 8.10 (s, 1H), 48 (s, 1H).

### Step 13c. Ethyl 2-(4-(3-Ethynylphenylamino)quinazolin-6-yloxy)acetate (Compound 0212-19)

The title compound 0212-19 (0.2 g, yield: 75%) was prepared from 4-(3-ethynylphenylamino)quinazolin-6-ol 0211 and ethyl 2-bromoacetate using a procedure similar to that described for compound 0210-13 (Example 9): LC-MS m/z 322 [M+1], mp 181-182° C.) ¹H-NMR (DMSO) δ1.28 (t. 3H), 4.20 (q, 2H), 4.25 (s, 1H) 4.32 (s, 2H), 7.23 (d, 1H), 7.41 (t, 1H), 7.57 (dd, 1H), 7.74 (d, 1H), 7.91 (d, 1H), 7.95 (m, 1H), 8.10 (s, 1H), 8.48 (s, 1H).

### Step 13d. 2-(4-(3-Ethynylphenylamino)quinazolin-6-yloxy)-N-hydroxyacetamide (Compound 19)

The title compound 12 (40 mg) was prepared from ethyl 2-(4-(3-ethynylphenylamino)quinazolin-6-yloxy)acetate 0212-19 using a procedure similar to that described for compound 13 (Example 9): LC-MS m/z 335 [M+1], mp: 189-191° C., ¹H-NMR (DMSO) δ4.27 (s. 1H), 4.69 (s, 2H), 7.39 (d, 1H), 7.49 (t, 1H), 7.76 (m, 2H), 7.83 (m, 2H), 7.88 (s, 1H), 8.10 (s, 1H), 8.82 (m, 1H).

### Example 14

### Preparation of 4-(4-(3-ethynylphenylamino)quinazolin-6-yloxy)-N-hydroxybutanamide (Compound 21)

### Step 14a. Ethyl 4-(4-(3-ethynylphenylamino)quinazolin-6-yloxy)butanoate (Compound 0212-21)

The title compound 0212-21 (0.2 g, 78%) was prepared from compound 4-(3-ethynylphenylamino)quinazolin-6-ol (0211) and ethyl 4-bromobutanoate using a procedure similar to that described for compound 0210-13 (Example 9): LC-MS m/z 376 [M+1], ¹H-NMR (DMSO) δ1.12 (t. 3H), 1.79 (m, 2H), 2.32 (t, 2H), 4.04 (q, 2H), 4.16 (t, 2H), 4.21 (s, 1H), 7.02 (dd, 1H), 7.21 (d, 1H), 7.39 (dd, 1H), 7.70 (t, 1H), 7.88 (s, 1H), 8.00 (m, 1H), 8.51 (s, 1H), 8.65 (s, 1H).

### Step 14b. 4-(4-(3-Ethynylphenylamino)quinazolin-6-yloxy)-N-hydroxybutanamide (Compound 21)

The title compound 21 (50 mg) was prepared from ethyl 4-(4-(3-ethynylphenylamino)quinazolin-6-yloxy)butanoate (0212-21) using a procedure similar to that described for compound 13 (Example 9): LC-MS m/z 363 [M+1], mp 182-186° C., ¹H-NMR (DMSO) δ2.02 (m, 2H), 2.20 (t, 2H), 4.16 (t, 2H), 4.20 (s, 1H), 7.24 (d, 1H), 7.43 (t, 1H), 7.52 (dd, 1H), 7.75 (d, 1H), 7.94 (m, 2H), 8.06 (s, 1H), 8.53 (s, 1H).

### Example 15

### Preparation of 6-(4-(3-ethynylphenylamino)quinazolin-6-yloxy)-N-hydroxyhexanamide (Compound 23)

### Step 15a. 6-(4-(3-Ethynylphenylamino)quinazolin-6-yloxy)hexanoate (Compound 0212-23)

The title compound ethyl 6-(4-(3-ethynylphenylamino)quinazolin-6-yloxy)hexanoate (0212-23) (0.3 g, 64%) was prepared from compound 4-(3-ethynylphenylamino)quinazolin-6-ol (0211) and ethyl 6-bromohexanoate using a procedure similar to that described for compound 0210-13 (Example 9): LC-MS m/z 404 [M+1].

### Step 15b. 6-(4-(3-Ethynylphenylamino)quinazolin-6-yloxy)-N-hydroxyhexanamide (Compound 23)

The title compound 23 (50 mg) was prepared from ethyl 6-(4-(3-ethynylphenylamino)quinazolin-6-yloxy)hexanoate (0212-23) using a procedure similar to that described for compound 13 (Example 9): LC-MS m/z 391 [M+1], mp 176-182° C., ¹H-NMR (DMSO) δ1.46 (m, 2H), 1.60 (m, 2H), 1.81 (m, 2H), 2.00 (t, 2H), 4.15 (t, 2H), 4.20 (s, 1H), 7.24 (d, 1H), 7.43 (t, 1H), 7.52 (dd, 1H), 7.72 (d, 1H), 7.92 (m, 2H), 8.04 (s, 1H), 8.53 (s, 1H).

### Example 16

### 4-(4-(3-Chloro-4-fluorophenylamino)-7-methoxyquinazolin-6-yloxy)-N-hydroxybutanamide (Compound 4)

### Step 16a. Ethyl 4-(4-(3-chloro-4-fluorophenylamino)-7-methoxyquinazolin-6-yloxy)butanoate (Compound 0110-4)

The title compound 0110-4 was prepared as a yellow solid (600 mg, 88.4%) from compound 0109 from step 1f (500 mg, 1.56 mmol) and methyl 5-bromopentanoate (320 mg, 1.64 mmol) using a procedure similar to that described for compound 0110-1 (Example 1): LCMS: 434 [M+1]; ¹H NMR (CDCl₃): δ 1.80˜1.97 (m, 4H), 2.48 (t, J=6.6 Hz, 2H), 3.67 (s, 3H), 3.97 (s, 3H), 4.18 (t, J=7.2 Hz, 2H), 7.14 (t, J=8.7 Hz, 1H), 7.24 (s, 1H), 7.29 (s, 1H), 7.66˜7.11 (m, 1H), 7.96 (dd, J=6.9 Hz, 2.7 Hz, 1H), 8.03 (s, 1H), 8.66 (s, 1H).

### Step 16b. 4-(4-(3-Chloro-4-fluorophenylamino)-7-methoxyquinazolin-6-yloxy)-N hydroxybutanamide (Compound 4)

The title compound 4 was prepared as a white solid (140 mg, 35%) form compound 0110-4 (400 mg, 0.92 mmol) using a procedure similar to that described for compound 1 (Example 1): LCMS: 435 [M+1]⁺; ¹H NMR (DMSO-d⁶): δ 1.69˜1.84 (m, 4H), 2.07 (t, J=6.6 Hz, 2H), 3.94 (s, 3H), 4.15 (t, J=6.0 Hz, 2H), 7.21 (s, 1H), 7.45 (t, J=9.0 Hz, 1H), 7.78˜7.83 (m, 2H), 8.13 (dd, J=6.9 Hz, 2.4 Hz, 1H), 8.03 (s, 1H), 8.50 (s, 1H), 8.72 (s, 1H), 9.54 (s, 1H), 10.41 (s, 1H).

### Example 17

### 5-(4-(3-Ethynylphenylamino)-7-methoxyquinazolin-6-yloxy)-N-hydroxypentanamide (Compound 10)

### Step 17a. Methyl 5-(4-(3-ethynylphenylamino)-7-methoxyquinazolin-6-yloxy)pentanoate (Compound 0113-10)

The title compound 0113-10 was prepared as a yellow solid (500 mg, 72%) from compound 0112 (500 mg, 1.7 mmol) and methyl 5-bromopentanoate (211 mg, 0.89 mmol) using a procedure similar to that described for compound 0110-1 (Example 1): LCMS: 406 [M+1]⁺.

### Step 17b. 5-(4-(3-Ethynylphenylamino)-7-methoxyquinazolin-6-yloxy)-N-hydroxypentanamide (Compound 10)

The title compound 10 was prepared as a white solid (200 mg, 40%) from compound 0113-10 (500 mg, 1.23 mmol) using a procedure similar to that described for compound 1 (Example 1): LCMS: 407 [M+1]⁺; ¹H NMR (DMSO-d₆): δ 1.71˜1.85 (m, 4H), 2.07 (t, J=7.2 Hz, 2H), 3.93 (s, 3H), 4.16 (t, J=6.3 Hz, 2H), 4.20 (s, 1H), 7.19 (m, 2H), 7.41 (t, J=8.1 Hz, 1H), 7.84 (s, 1H), 7.90 (dd, J=8.4 Hz, 1.2 Hz, 1H), 8.00 (t, J=1.8 Hz, 1H), 8.50 (s, 1H), 8.72 (s, 1H), 9.48 (s, 1H), 10.40 (s, 1H).

### Example 18

### Preparation of 5-(4-(3-chloro-4-fluorophenylamino)quinazolin-6-yloxy)-N-hydroxypentanamide (Compound 16)

### Step 18a. ethyl 5-(4-(3-chloro-4-fluorophenylamino)quinazolin-6-yloxy)pentanoate (compound 0210-16)

The title compound 0210-16 (0.2 g, 68%) was prepared from compound 0209 4-(3-chloro-4-fluorophenylamino)quinazolin-6-ol (0.2 g, 0.69 mmol) and methyl 5-bromopentanoate (0.14 g, 0.69 mmol) using a procedure similar to that described for compound 0210-13 (Example 9): LCMS 376 [M+1]⁺.

### Step 18b. 5-(4-(3-chloro-4-fluorophenylamino)quinazolin-6-yloxy)-N-hydroxypentanamide (Compound 16)

The title compound 16 (24 mg, 67%) was prepared from compound 0210-16 (37 mg, 0.09 mmol) using a procedure similar to that described for compound 13 (Example 9): mp: 85.9° C.; LCMS 405 [M+1]⁺, ¹H NMR (DMSO-d₆) δ 1.74 (m, 4H), 2.04 (t, J=7.5 Hz, 2H), 4.14 (t, J=6 Hz, 2H), 7.44 (t, J=9 Hz, 1H), 7.51 (dd, J=9 Hz, J=2.4 Hz, 1H), 7.73 (d, J=8.7 Hz, 1H), 7.82 (m, 1H), 7.88 (d, J=2.4, 1H) 8.16 (dd, J=6.9 Hz, J=2.7 Hz 1H), 8.52 (s, 1H), 8.69 (s, 1H), 9.67 (s, 1H), 10.38 (s, 1H).

### Example 19

### Preparation of 7-(4-(3-ethynylphenylamino)quinazolin-6-yloxy)-N-hydroxyheptanamide (Compound 24)

### Step 19a. Ethyl 7-(4-(3-ethynylphenylamino)quinazolin-6-yloxy)heptanoate (Compound 0212-24)

The title compound 0212-24 (0.21 g, 58%) was prepared from compound 4-(3-ethynylphenylamino)quinazolin-6-ol (0211) (0.23 g, 0.86 mmol) and ethyl 7-bromoheptanoate (0.20 g, 0.86 mmol) using a procedure similar to that described for compound 0210-13 (Example 9): LCMS 418 [M+1]⁺.

### Step 19b. 7-(4-(3-Ethynylphenylamino)quinazolin-6-yloxy)-N-hydroxyheptanamide (Compound 24)

The title compound 24 (50 mg, 42%) was prepared from compound 0212-24 (123 mg, 0.29 mmol) using a procedure similar to that described for compound 13 (Example 9): LCMS 405 [M+1]⁺, ¹H NMR (DMSO-d₆): δ 1.44 (m, 2H), 1.48 (m, 2H), 1.59 (m, 2H), 1.67 (m, 2H), 2.11 (t, J=7.2 Hz, 2H), 3.50 (s, 1H), 4.17 (t, J=6.3 Hz, 2H), 7.28 (d, J=7.5 Hz, 1H), 7.37 (t, J=6.9 Hz, 1H), 7.48 (d, J=9.0 Hz, 1H), 7.78 (dd, J=21.3 Hz, J=7.8 Hz, 1H), 7.93 (s, 1H), 7.92 (m, 2H), 8.45 (s, 1H).

### Example 20

### Example 1

### Synthesis of 7-(4-(3-Chloro-4-fluorophenylamino)-7-(2-methoxyethoxy)quinazolin-6-yloxy)-N-hydroxyheptanamide (Compound 30)

### Step 20a. Ethyl 3-hydroxy-4-(2-methoxyethoxy)benzoate (Compound 0402-30)

To a solution of 0401 (1.82 g, 10.0 mmol) in N,N-dimethylformamide (20 mL) was added potassium carbonate (1.38 g, 10.0 mmol). The mixture was stirred for 15 minutes and then a solution of 2-methoxyethyl 4-methylbenzenesulfonate (2.30 g, 10.0 mmol) in N,N-dimethylformamide (5 mL) was added slowly dropwise. The mixture was stirred 48 hours at room temperature and filtered. The filtrate was concentrated in vacuo and the residue was dissolved in ethyl acetate (30 mL) then the organic layer was washed with brine (20 mL×3) and dried over sodium sulfate, filtered and evaporated to give the title product 0402-30 as a white solid (1.2 g, 50%): LCMS: 241 [M+1]⁺. ¹H NMR (DMSO-d₆) δ 1.26 (t, J=7.5 Hz, 3H), 3.65 (m, J=1.5 Hz, 2H), 4.11 (m, J=4.5 Hz, 2H), 4.21 (m, J=4.5 Hz, 2H), 7.00 (d, J=9 Hz, 1H), 7.37 (m, J=2 Hz, 2H), 9.40 (s, 1H).

### Step 20b. Ethyl 3-(7-ethoxy-7-oxoheptyloxy)-4-(2-methoxyethoxy)benzoate (Compound 0403-30)

Compound 0402-30 (204.0 mg, 0.85 mmol) and ethyl 7-bromoheptanoate (201.0 mg, 0.85 mmol) and potassium carbonate (353.0 mg, 2.50 mmol) in N,N-dimethylformamide (5 mL) was stirred at 60° C. for 3 hours. The mixture was filtrated. The filtrate was concentrated in vacuo and the residue was dissolved in ethyl acetate (30 mL) then the organic layer was washed with brine (20 mL×3) and dried over sodium sulfate, filtered and evaporated to give the title product 0403-30 as a yellow solid (325 mg, 96%): LCMS: 397 [M+1]⁺.

### Step 20c. Ethyl 5-(7-ethoxy-7-oxoheptyloxy)-4-(2-methoxyethoxy)-2-nitrobenzoate (Compound 0404-30)

Compound 0403-30 (325.0 mg, 0.82 mmol) was dissolved in acetic acid (2 mL) and stirred at room temperature. Then fuming nitric acid (0.39 g, 6.0 mmol) was added slowly dropwise. The mixture was stirred at room temperature for 2 hours. Poured into ice-water (50 mL) and extracted with ethyl acetate (20 mL×2). The combined organic layer was washed with aqueous NaHCO₃ solution (10 mL×3) and brine (10 mL×3) and dried over sodium sulfate, filtered and evaporated to give the title product 0404-30 as a yellow oil (330 mg, 100%): LCMS: 442 [M+1]⁺.

### Step 20d. Ethyl 2-amino-5-(7-ethoxy-7-oxoheptyloxy)-4-(2-methoxyethoxy)benzoate (Compound 0405-30)

A mixture of 0404-30 (370.0 mg 0.82 mmol), ethanol (4.4 mL), water (3 mL) and hydrogen chloride (0.08 mL) was stirred to form a clear solution. The powder iron (459.0 mg, 8.2 mmol) was added. The mixture was stirred at reflux for 30 minutes and cooled to room temperature, adjust pH to 8 with 10% sodium hydroxide solution in ice-water bath. The mixture was filtered and the filtrate was concentrated to remove ethanol and was then extracted whit ethyl acetate (20 mL×2). The combined organic layer was washed with brine (10 mL×3) and dried over sodium sulfate, filtered and evaporated to give the title product 0405-30 as a yellow oil (315 mg, 93%): LCMS: 412 [M+1]⁺.

### Step 20e. Ethyl 7-(7-(2-methoxyethoxy)-4-oxo-3,4-dihydroquinazolin-6-yloxy)heptanoate (Compound 0406-30)

A mixture of compound 0405-30 (315.0 mg, 0.76 mmol), ammonium formate (48.0 mg, 0.76 mmol) and formamide (2.46 mL) was stirred at 190° C. for 3 hours. The reaction mixture was cooled to room temperature. The formamide was removed under reduce pressure, and the residue was dissolved in ethyl acetate (30 mL). The organic layer was washed with brine (10 mL×5) and dried over sodium sulfate, filtered and evaporated to give the title product 0406-30 as a white solid (235 mg, 98%): LCMS: 393 [M+1]⁺.

### Step 20f. Ethyl 7-(4-chloro-7-(2-methoxyethoxy)quinazolin-6-yloxy)heptanoate (Compound 0407-30)

A mixture of product 0406-30 (235.0 mg, 0.6 mmol) and phosphoryl trichloride (3 mL) was stirred at reflux for 4 hours. When a clear solution was obtained, the excessive phosphoryl trichloride was removed under reduced pressure. The residue was dissolved in ethyl acetate (30 mL) and the organic layer was washed in turn with water (10 mL×2), aqueous NaHCO₃ solution (10 mL×2) and brine (20 mL×1), dried over sodium sulfate, filtered and evaporated to give the title product 0407-30 as a yellow solid (233 mg, 94%): LCMS: 411 [M+1]⁺.

### Step 20g. Ethyl 7-(4-(3-chloro-4-fluorophenylamino)-7-(2-methoxyethoxy)-quinazolin-6-yloxy)heptanoate (Compound 0408-30)

A mixture of product 0407-30 (117.0 mg, 0.28 mmol) and 3-chloro-4-fluorobenzenamine (50.0 mg, 0.34 mmol) in isopropanol (3 mL) was stirred at reflux overnight. The mixture was cooled to room temperature and resulting precipitate was isolated, washed with isopropanol and ether. The solid was then dried to give the title compound 0408-30 as a yellow solid (102 mg, 70%): LCMS: 520 [M+1]⁺.

### Step 20h. 7-(4-(3-Chloro-4-fluorophenylamino)-7-(2-methoxyethoxy)quinazolin-6-yloxy)-N-hydroxyheptanamide (Compound 30)

The freshly prepared hydroxylamine solution (3 mL, 2.0 mmol) was placed in 25 mL flask. Compound 408-30 (102.0 mg, 0.2 mmol) was added and stirred at room temperature for 24 hours. The mixture was neutralized with acetic acid/methanol. The mixture was concentrated under reduce pressure. The residue was purified by preparation HPLC to give the title compound 30 as a yellow solid (85 mg, 84%): LCMS: 507 [M+1]⁺; ¹H NMR (DMSO-d₆) δ 1.33 (m, 2H), 1.50 (m, 4H), 1.79 (s, 2H), 1.94 (t, 2H), 3.29 (s, 3H), 3.72 (s, 2H), 4.11 (s, 2H), 4.25 (s, 2H), 7.19 (s, 1H), 7.42 (t, 1H), 7.79 (s, 1H), 8.10 (d, 1H), 8.47 (s, 1H), 8.65 (s, 1H), 9.52 (s, 1H), 10.33 (s, 1H).

### Example 21

### Preparation of 7-(4-(3-Ethynylphenylamino)-7-(2-methoxyethoxy)quinazolin-6-yloxy)-N-hydroxyheptanamide (Compound 36)

### Step 21a. Ethyl 7-(4-(3-ethynylphenylamino)-7-(2-methoxyethoxy)quinazolin-6-yloxy)heptanoate (Compound 0408-36)

A mixture of product 0407-30 (102.0 mg, 0.25 mmol) and 3-ethynylbenzenamine (35.0 mg, 0.3 mmol) in isopropanol (3 mL) was stirred at reflux overnight. The mixture was cooled to room temperature and resulting precipitate was isolated, washed with isopropanol and ether. The solid was then dried to give the title compound 0408-36 as a yellow solid (88 mg, 72%): LCMS: 491 [M+1]⁺.

### Step 21b. 7-(4-(3-Ethynylphenylamino)-7-(2-methoxyethoxy)quinazolin-6-yloxy)-N-hydroxyheptanamide (Compound 36)

The freshly prepared hydroxylamine solution (3 mL, 2 mmol) was placed in 25 mL flask. Compound 0408-36 (88.0 mg, 0.18 mmol) was added to this solution and stirred at room temperature for 24 hours. The mixture was neutralized with acetic acid/methanol and was concentrated under reduce pressure. The residue was purified by preparative HPLC to give the title compound 36 as a white solid (40 mg, 47%): LCMS: 479 [M+1]⁺; ¹H NMR (DMSO-d₆) δ 1.33 (m, 2H), 1.50 (m, 4H), 1.79 (s, 1H), 1.94 (t, 2H), 3.72 (s, 2H), 4.11 (s, 2H), 4.25 (s, 2H), 7.19 (s, 1H), 7.42 (t, 1H), 7.79 (s, 1H), 8.10 (d, 1H), 8.47 (s, 1H), 8.65 (s, 1H), 9.52 (s, 1H), 10.33 (s, 1H).

### Example 22

### Preparation of N¹-(4-(3-chloro-4-fluorophenylamino)-7-methoxyquinazolin-6-yl)-N⁵-hydroxyglutaramide (Compound 38)

### Step 22a. 7-Chloroquinazolin-4(3H)-one (Compound 0302)

A mixture of compound 0301 (17.2 g, 100 mmol) and formamide (20 mL) was stirred at 130° C. for 30 minutes and to 190° C. for 4 hours. The mixture was allowed to cool to room temperature. It was then poured into a mixture of ice and water. The precipitate was isolated, washed with water and dried to give the title compound 0302 (15.8 g, 87.7%). ¹H NMR (DMSO-d₆): δ 7.65 (dd, 1H), 7.72 (d, 1H), 8.12 (d, 1H), 8.36 (s, 1H).

### Step 22b. 7-Chloro-6-nitroquinazolin-4(3H)-one (compound 0303)

Compound 0302 (18.0 g, 100 mmol) was added portionwise to a stirred mixture of concentrated sulfuric acid (60 mL) and fuming nitric acid (60 mL) which had been cooled to 0° C., the mixture was stirred at ambient temperature for 1 hour and then heated to 45° C. overnight. The mixture was poured into the mixture of ice and water. The precipitate was isolated, washed with water and dried. Recrystallization from acetic acid to give the title compound 0303 (14.1 g, 62.7%). ¹H NMR (DMSO-d₆): δ 8.00 (s, 1H), 8.27 (s, 1H), 8.65 (s, 1H), 12.70 (s, 1H).

### Step 22c. 7-Methoxy-6-nitroquinazolin-4(3H)-one (compound 0304)

A mixture of compound 0303 (4.0 g, 18.0 mmol) and sodium (2.4 g, 45 mmol) in methanol (50 mL) was heated at 100° C. in a sealed pressure vessel for 20 hours. The solution was neutralized with acetic acid and diluted with water to give the title compound 0304 (3.0 g, 77%). ¹H NMR (DMSO-d₆): δ4.10 (s, 3H), 7.40 (s, 1H), 8.24 (s, 1H), 8.50 (s, 1H), 12.67 (s, 1H).

### Step 22d. 4-Chloro-7-methoxy-6-nitroquinazoline (compound 0305)

Compound 0304 (3.8 g, 17.2 mmol) was suspended in POCl₃ (75 mL), the mixture was heated to reflux for 4 hours. The additional POCl₃ was removed in a vacuum. The residue was dissolved in a mixture of dichloromethane (50 mL) and aqueous NaHCO₃ (50 mL). The organic layer was dried and the solvent was removed to give the title compound 0305 (3.4 g, 83%). ¹H NMR (DMSO-d₆): δ 4.05 (s, 3H), 7.44 (s, 1H), 8.27 (s, 1H), 8.53 (s, 1H).

### Step 22e. N-(3-chloro-4-fluorophenyl)-7-methoxy-6-nitroquinazolin-4-amine Hydrochloride (compound 0307)

A mixture of compound 0305 (3.4 g, 14.2 mmol) and 3-chloro-4-fluoroaniline (0406) (2.2 g, 15.2 mmol) and isopropanol (120 mL) was stirred at reflux for 3 hours. The mixture was cooled to ambient temperature and the precipitate was isolated, washed with methanol and ether and then dried to give the title compound 0307 (4.66 g, 85%). ¹H NMR (DMSO-d₆): δ 4.10 (s, 3H), 7.55 (dd, 2H), 7.74 (m, 1H), 8.07 (dd, 1H), 8.90 (s, 1H), 9.55 (s, 1H), 11.6 (s, 1H).

### Step 22f. N-(3-chloro-4-fluorophenyl)-7-methoxy-6-nitroquinazolin-4-amine (Compound 0308)

A mixture of compound 0307 (3.5 g, 10.0 mmol) and iron dust (11.2 g, 200.0 mmol) and ethanol (100 mL) and concentrated hydrochloric acid (2 mL), and water (30 mL) was heated to reflux for 1 hour. Removed iron dust by filtration. The filtrate was concentrated to 1/5 volume. The precipitate was isolated and dried to give the title compound 0308 (2.2 g, 69%). ¹H NMR (DMSO-d₆): δ 3.97 (s, 3H), 5.38 (s, 2H), 7.10 (s, 1H), 7.36 (s, 1H), 7.39 (t, 1H), 7.80 (m, 1H), 8.08 (dd, 1H), 8.38 (s, 1H), 9.39 (s, 1H).

### Step 22g. Methyl 3-(4-(3-chloro-4-fluorophenylamino)-7-methoxyquinazolin-6-ylcarbamoyl)propanoate (Compound 0310-38)

The compound 0308 (500.0 mg, 1.57 mmol) and triethylamine (165.0 mg, 1.65 mmol) was dissolved in dichloromethane (50 mL). The mixture was cooled to 0° C. and the solution of methyl 5-chloro-5-oxopentanoate (270 mg, 1.65 mmol) in dichloromethane (5 mL) was added into above mixture dropwise under 0° C. in 20 minutes. The reaction mixture was allowed to stir at ambient temperature for 1 hour. The mixture was washed with water (50 mL×2) and brine (50 mL). The organic layer was dried over MgSO₄, filtered and concentrated to give the title compound 0310-38 (550 mg, 78%), LCMS: 448 [M+1]⁺.

### Step 22h. N¹-(4-(3-chloro-4-fluorophenylamino)-7-methoxyquinazolin-6-yl)-N⁵-hydroxyglutaramide (Compound 38)

To a stirred solution of hydroxylamine hydrochloride (4.67 g, 67 mmol) in methanol (24 mL) at 0° C. was added a solution of potassium hydroxide (5.61 g, 100 mmol) in methanol (14 mL). After addition, the mixture was stirred for 30 minutes at 0° C. and was allowed to stand at low temperature. The resulting precipitate was isolated, and the solution was prepared to give free hydroxylamine.

The above freshly prepared hydroxylamine solution (5.6 mL, 10.0 mmol) was placed in 10 mL flask. Compound 0310-38 (550.0 mg, 1.23 mmol) was added to this solution and stirred at 0° C. for 10 minutes and was allowed to warm to room temperature. The reaction process was monitored by TLC. The mixture was neutralized with acetic acid. The mixture was concentrated under reduce pressure. The residue was purified by preparative HPLC to give the title compound 38 as a grey solid (250 mg, 45%): LCMS: 448 [M+1]⁺; ¹H NMR (DMSO-d₆): δ 1.85 (m, 2H), 2.06 (t, J=7.5 Hz, 2H), 2.48 (t, J=7.2 Hz, 2H), 4.00 (s, 3H), 7.24 (s, 1H), 7.42 (t, J=9.0 Hz, 1H), 7.80 (m, 1H), 8.10 (dd, J=7.2 Hz, 2.7 Hz, 1H), 8.52 (s, 1H), 8.70 (s, 1H), 8.82 (s, 1H), 9.48 (s, 1H). 9.79 (s, 1H), 10.40 (s, 1H).

### Example 23

### Preparation of N¹-(4-(3-chloro-4-fluorophenylamino)-7-methoxyquinazolin-6-yl)-N⁸-hydroxyoctanediamide (Compound 40)

### Step 23a. Methyl 8-(4-(3-chloro-4-fluorophenylamino)-7-methoxyquinazolin-6-ylamino)-8-oxooctanoate (Compound 0310-40)

The title compound 0310-40 was prepared as a yellow solid (350 mg, 78%) from compound 0308 (319 mg, 1.0 mmol) and methyl 8-chloro-8-oxooctanoate (227 mg, 1.1 mmol) using a procedure similar to that described for compound 0310-38 (Example 22): LCMS: 489 [M+1]⁺.

### Step 23b. N¹-(4-(3-chloro-4-fluorophenylamino)-7-methoxyquinazolin-6-yl)-N⁸-hydroxyoctanediamide (Compound 40)

The title compound 40 was prepared as a yellow solid (120 mg, 30%) from compound 0310-38 (400 mg, 0.8 mmol) using a procedure similar to that described for compound 38 (Example 22): LCMS: 490 [M+1]⁺; ¹H NMR (DMSO-d₆): δ 1.29 (m, 4H), 1.48 (m, 2H), 1.59 (m, 2H), 1.93 (t, J=7.2 Hz, 2H), 2.45 (t, J=7.2 Hz, 2H), 4.00 (s, 3H), 4.18 (s, 1H), 7.26 (s, 1H), 7.41 (t, J=9.0 Hz, 1H), 7.74 (m, 1H), 8.08 (d, J=1.2 Hz, 1H), 8.54 (s, 1H), 8.66 (s, 1H), 8.83 (s, 1H), 9.46 (s, 1H), 9.95 (s, 1H), 10.33 (s, 1H).

### Example 24

### Preparation of N¹-(4-(3-ethynylphenylamino)-7-methoxyquinazolin-6-yl)-N⁵-hydroxyglutaramide (Compound 42)

### Step 24a. N-(3-ethynylphenyl)-7-methoxy-6-nitroquinazolin-4-amine Hydrochloride (Compound 0307-42)

The title compound 0307-42 was prepared as a yellow solid (4.7 g, 84.5%) from compound 0305 (350 mg, 0.78 mmol) and 3-ethynylbenzenamine (2.34 g, 20.0 mmol) using a procedure similar to that described for compound 0306-38 (Example 22): LCMS: 321 [M+1]⁺; ¹H NMR (DMSO-d₆): δ 4.11 (s, 3H), 4.24 (s, 1H), 7.42 (d, 1H), 7.50 (t, 1H), 7.61 (s, 1H), 7.79 (d, 1H), 7.93 (m, 1H), 8.93 (s, 1H), 9.57 (s, 1H), 11.56 (bs, 1H).

### Step 24b. N⁴-(3-ethynylphenyl)-7-methoxyquinazoline-4,6-diamine (Compound 0309-42)

The title compound 0309-42 was prepared as a yellow solid (2.0 g, 69%) from compound 0307-42 (3.2 g, 10.0 mmol) using a procedure similar to that described for compound 0308-38 (Example 22): LCMS: 291 [M+1]⁺; ¹H NMR (DMSO-d₆): δ 3.95 (s, 3H), 4.14 (s, 1H), 5.33 (s, 2H), 7.08 (m, 2H), 7.34 (m, 2H), 7.88 (m, 1H), 8.04 (s, 1H), 8.36 (s, 1H), 9.29 (s, 1H).

### Step 24c. Methyl 5-(4-(3-ethynylphenylamino)-7-methoxyquinazolin-6-ylamino)-5-oxopentanoate (Compound 0311-42)

The title compound 0311-42 was prepared as a yellow solid (450 mg, 77%) from compound 0309-42 (407 mg, 1.4 mmol) and methyl 5-chloro-5-oxopentanoate (254 mg, 1.54 mmol) using a procedure similar to that described for compound 0310-38 (Example 22): LCMS: 419 [M+1]⁺.

### Step 24d. N¹-(4-(3-ethynylphenylamino)-7-methoxyquinazolin-6-yl)-N⁵-hydroxyglutaramide (Compound 42)

The title compound 42 was prepared as a yellow solid (100 mg, 47%) from compound 0311-42 (211 mg, 0.5 mmol) using a procedure similar to that described for compound 38 (Example 22).

### Example 25

### Preparation of N¹-(4-(3-ethynylphenylamino)-7-methoxyquinazolin-6-yl)-N⁶-hydroxyadipamide (Compound 43)

### Step 25a. Methyl 6-(4-(3-ethynylphenylamino)-7-methoxyquinazolin-6-ylamino)-6-oxohexanoate (Compound 0311-43)

The title compound 0311-43 was prepared as a yellow solid (530 mg, 71%) from compound 0309-42 (500 mg, 1.72 mmol) and methyl 6-chloro-6-oxohexanoate (323 mg, 1.81 mmol) using a procedure similar to that described for compound 0311-42 (Example 24): LCMS: 433 [M+1]⁺.

### Step 25b. N¹-(4-(3-ethynylphenylamino)-7-methoxyquinazolin-6-yl)-N⁶-hydroxyadipamide (Compound 43)

The title compound 43 was prepared as a yellow solid (105 mg, 24%) from compound 0311-43 (432 mg, 1.0 mmol) using a procedure similar to that described for compound 42 (Example 24): m.p.: 191.2˜196.7° C.; LCMS: 434 [M+1]⁺; ¹H NMR (DMSO-d₆): δ 1.58 (m, 4H), 1.98 (t, J=6.3 Hz, 2H), 2.44 (m, 2H), 3.99 (s, 3H), 4.16 (s, 1H), 7.18 (d, J=7.8 Hz, 1H), 7.25 (s, 1H), 7.37 (t, J=8.1 Hz, 1H), 7.84 (d, J=8.4 Hz, 1H), 7.98 (s, 1H), 8.51 (s, 1H), 8.66 (s, 1H), 8.82 (s, 1H), 9.42 (s, 1H), 9.73 (s, 1H), 10.35 (s, 1H).

### Example 26

### N¹-(4-(3-ethynylphenylamino)-7-methoxyquinazolin-6-yl)-N⁸-hydroxyoctanediamide (Compound 44)

### Step 26a. Methyl 8-(4-(3-ethynylphenylamino)-7-methoxyquinazolin-6-ylamino)-8-oxooctanoate (Compound 0311-44)

The title compound 0311-44 was prepared as a yellow solid (150 mg, 78%) from compound 0309-42 (120 mg, 0.4 mmol) and methyl 8-chloro-8-oxooctanoate (91 mg, 0.44 mmol) using a procedure similar to that described for compound 0311-42 (Example 24): LCMS: 461 [M+1]⁺.

### Step 26b. N¹-(4-(3-ethynylphenylamino)-7-methoxyquinazolin-6-yl)-N⁸-hydroxyoctanediamide (Compound 44)

The title compound 44 was prepared as a yellow solid (30 mg, 20%) from compound 0311-44 (150 mg, 0.3 mmol) using a procedure similar to that described for compound 42 (Example 24): LCMS: 462 [M+1]⁺; ¹H NMR (DMSO-d₆): δ 1.30 (m, 4H), 1.51 (m, 2H), 1.62 (m, 2H), 1.95 (t, J=7.2 Hz, 2H), 2.45 (t, J=7.2 Hz, 2H), 4.00 (s, 3H), 4.18 (s, 1H), 7.19 (d, J=7.2 Hz, 1H), 7.26 (s, 1H), 7.38 (t, J=7.8 Hz, 1H), 7.86 (d, J=7.8 Hz, 1H), 7.99 (s, 1H), 8.52 (s, 1H), 8.83 (s, 1H), 9.44 (s, 1H).

### Example 27

### Preparation of (E)-3-(4-(2-(4-(3-chloro-4-fluorophenylamino)-7-methoxyquinazolin-6-yloxy)ethoxy)phenyl)-N-hydroxyacrylamide (Compound 66)

### Step 27a. (E)-Methyl 3-(4-hydroxyphenyl)acrylate (Compound 0501-66)

A mixture of 4-hydroxycinnamic acid (8.2 g, 50 mmol) and a drop of H₂SO₄ in methanol (30 mL) was heated to reflux overnight. Then the solvent was evaporated, the residue was dissolved in ethyl acetate, washed with saturated NaHCO₃ solution twice, brine, dried over MgSO₄, concentrated to give the title compound 0501-66 as white solid (8.7 g, 98%): LCMS: 179 [M+1]⁺.

### Step 27b. (E)-Methyl 3-(4-(2-(tosyloxy)ethoxy)phenyl)acrylate (Compound 0502-66)

A mixture of compound 0501-66 (5.0 g, 28.0 mmol) and 2-bromoethanol (3.9 g, 62.0 mmol) and potassium carbonate in N,N-dimethylformamide was stirred at 80° C. for 24 hours. The reaction process was monitored by TLC. The mixture was filtrated. The filtrate was concentrated under reduce pressure. The residue was wash with diethyl ether and dried to give (E)-methyl 3-(4-(2-hydroxyethoxy)phenyl)-acrylate as yellow solid (1.6 g, 26.0%): LCMS: 223 [M+1]⁺.

To a mixture of triethylamine (0.3 g, 3 mol) and dichloromethane (20 mL) was added tosyl chloride (285 mg, 1.5 mmol) batchwise and stirred for 0.5 hour. Compound (E)-methyl 3-(4-(2-hydroxyethoxy)phenyl)acrylate (333 mg, 1.5 mmol) was added into above mixture and heated to reflux for 24 hours. The reaction mixture was added saturated ammonium chloride solution and the organic layer was separated and washed by brine, dried (MgSO₄), evaporated to give compound 0502-66 as white solid (200 mg, 36%): LCMS: 377 [M+1]⁺.

### Step 27c. (E)-Methyl 3-(4-(2-(4-(3-chloro-4-fluorophenylamino)-7-methoxyquinazolin-6-yloxy)ethoxy)phenyl)acrylate (Compound 0503-66)

A mixture of compound 0109 (176 mg, 0.55 mmol) and 0502-66 (152 mg, 0.94 mmol) and potassium carbonate in N,N-dimethylformamide was stirred at 80° C. for 24 hours. The reaction process was monitored by TLC. The mixture was filtrated. The filtrate was concentrated under reduce pressure. The residue was wash with diethyl ether and dried to give the title compound 0503-66 as yellow solid (281 mg, 98%): LCMS: 524 [M+1]⁺.

### Step 27d. (E)-3-(4-(2-(4-(3-Chloro-4-fluorophenylamino)-7-methoxyquinazolin-6-yloxy)ethoxy)phenyl)-N-hydroxyacrylamide (Compound 66)

The title compound 66 was prepared as a white solid (65 mg, 19%) from compound 0503-66 (346.0 mg, 0.66 mmol) using a procedure similar to that described for compound 1 (Example 1): LCMS: 525 [M+1]⁺; ¹H NMR (DMSO-d₆): δ 3.93 (s, 3H), 4.48 (s, 4H), 6.31 (d, J=16.2 Hz, 1H), 7.05 (d, J=8.1 Hz, 2H), 7.21 (s, 1H), 7.44 (t, J=9.0 Hz, 1H), 7.52 (d, J=8.1 Hz, 2H), 7.78 (d, J=10.2 Hz, 1H), 7.88 (m, 1H), 8.12 (dd, J=6.6 Hz, 2.7 Hz, 1H), 8.50 (s, 1H), 8.96 (s, 1H), 8.50 (s, 1H), 9.56 (s, 1H), 10.65 (s, 1H).

### Example 28

### Preparation of N¹-(4-(3-chloro-4-fluorophenylamino)-7-(2-methoxyethoxy)quinazolin-6-yl)-N⁵-hydroxyglutaramide (Compound 68)

### Step 28a. 7-(2-Methoxyethoxy)-6-nitroquinazolin-4(3H)-one (compound 0304-68)

Sodium (2.07 g, 90 mmol) was added to 2-methoxyethanol (125 mL) at 0° C. until sodium was dissolved. Compound 0303 (6.77 g, 30.0 mmol) was added to the solution. The mixture was stirred at 90° C. for 24 hours and was then adjusted to pH7 by acetic acid. Water (50 mL) was added to the mixture and resulting yellow precipitate was isolated, washed with water and dried to provide the title compound 0304-68 as a yellow solid (7.003 g, 88%): LCMS: 266 [M+1]⁺.

### Step 28b. 4-Chloro-7-(2-methoxyethoxy)-6-nitroquinazoline (compound 0305-68)

A mixture of product 0304-68 (5.30 g, 20.0 mmol) and phosphoryl trichloride (50 mL) was stirred at reflux for 5 hours. When a clear solution was obtained, the excessive phosphoryl trichloride was removed under reduced pressure. The residue was dissolved in ethyl acetate (100 mL) and the organic layer was washed in turn with water (30 mL×2), aqueous NaHCO₃ solution (20 mL×2) and brine (20 mL×1), dried over sodium sulfate, filtered and evaporated to give the title product 0305-68 as a yellow solid (5.31 g, 94%): LCMS: 284 [M+1]⁺.

### Step 28c. N-(3-chloro-4-fluorophenyl)-7-(2-methoxyethoxy)-6-nitroquinazolin-4-amine (compound 0306-68)

A mixture of product 0305-68 (5.31 g, 18.7 mmol) and 3-chloro-4-fluorobenzenamine (5.45 g, 37.4 mmol) in isopropanol (150 mL) was stirred at reflux overnight. The mixture was cooled to room temperature and resulting precipitate was isolated, washed with methanol and ether. The solid was then dried to give the title compound 0306-68 as a yellow solid (5.70 g, 77%): LCMS: 393 [M+1]⁺.

### Step 28d. N⁴-(3-chloro-4-fluorophenyl)-7-(2-methoxyethoxy)quinazoline-4,6-diamine (compound 0308-68)

A mixture of 0306-68 (5.70 g, 14.5 mmol), ethanol (165 mL), water (43.5 mL) and hydrogen chloride (2.9 mL) was stirred to form a clear solution. The powder iron (16.24 g, 290.0 mmol) was added. The mixture was stirred at reflux for 2 hours. Cooled to room temperature, adjusted pH to 11 with 10% sodium hydroxide solution in ice-water bath and was filtered. The filtrate was concentrated to remove ethanol and extracted whit ethyl acetate (100 mL×2), The combined organic layer was washed with brine (30 mL×3) and dried over sodium sulfate, filtered and evaporated to give the title product 0308-68 as a yellow solid (4.92 g, 93%): LCMS: 363 [M+1]⁺.

### Step 28e. Methyl 5-(4-(3-chloro-4-fluorophenylamino)-7-(2-methoxyethoxy)quinazolin-6-ylamino)-5-oxopentanoate (Compound 0310-68)

The methyl 5-chloro-5-oxopentanoate (0.198 g, 1.2 mmol) was added to a solution of compound 0308-68 (0.22 g, 0.6 mmol) in 30 mL of dichloromethane and triethylamine (0.48 g, 4.8 mmol). The mixture was stirred for 2 hours at 0° C. The reaction mixture was then washed with water and dried over sodium sulfate, filtered and evaporated to give the title product 0310-68 as a brown oil (270 mg, 92%): LCMS: 491 [M+1]⁺.

### Step 28f. N¹-(4-(3-chloro-4-fluorophenylamino)-7-(2-methoxyethoxy)quinazolin-6-yl)-N⁵-hydroxyglutaramide (Compound 68)

To a stirred solution of hydroxylamine hydrochloride (4.67 g, 67 mmol) in methanol (24 mL) at 0° C. was added a solution of potassium hydroxide (5.61 g, 100 mmol) in methanol (14 mL). After addition, the mixture was stirred for 30 minutes at 0° C. and was allowed to stand at low temperature. The resulting precipitate was isolated, and the solution was prepared to give free hydroxylamine.

The above freshly prepared hydroxylamine solution (6 mL, 4.0 mmol) was placed in 25 mL flask. Compound 0310-68 (270 mg, 0.55 mmol) was added to this solution and stirred at room temperature for 4 hours. The mixture was neutralized with acetic acid/methanol. The mixture was concentrated under reduce pressure. The residue was purified by preparative HPLC to give the title compound 68 as a yellow solid (220 mg, 75%): LCMS: 492 [M+1]⁺; ¹H NMR (DMSO-d₆): 1.83 (m, J=7.5 Hz, 2H), 2.05 (t, J=7.2 Hz, 2H), 2.43 (t, J=6.9 Hz, 2H), 3.31 (s, 3H), 3.76 (t, J=4.5 Hz, 2H), 4.32 (t, J=4.2 Hz, 2H), 7.28 (s, 1H), 7.40 (t, J=9 Hz, 1H), 7.77 (m, 1H), 8.10 (m, J=2.1 Hz, 1H), 8.50 (s, 1H), 8.67 (s, 1H), 8.752 (s, 1H), 9.33 (s, 1H), 9.77 (s, 1H), 10.38 (s, 1H).

### Example 29

### Preparation of N¹-(4-(3-chloro-4-fluorophenylamino)-7-(2-methoxyethoxy)quinazolin-6-yl)-N⁶-hydroxyadipamide (Compound 69)

### Step 29a. Methyl 6-(4-(3-chloro-4-fluorophenylamino)-7-(2-methoxyethoxy)quinazolin-6-ylamino)-6-oxohexanoate (Compound 0310-69)

The methyl 6-chloro-6-oxohexanoate (0.36 g, 1.76 mmol) was added to a solution of compound 0308-68 (0.15 g, 0.4 mmol), 25 mL of dichloromethane and triethylamine (0.162 g, 1.6 mmol). The reaction mixture was stirred for 2 hours at 0° C. The reaction was washed with water and dried over sodium sulfate, filtered and evaporated to give the title product 0310-69 as a brown oil (185 mg, 92%): LCMS: 505 [M+1]⁺.

### Step 29b. N¹-(4-(3-chloro-4-fluorophenylamino)-7-(2-methoxyethoxy)quinazolin-6-yl)-N⁶-hydroxyadipamide (compound 69)

The freshly prepared hydroxylamine solution (6 mL, 4 mmol) was placed in 25 mL flask. Compound 0310-69 (185 mg, 0.38 mmol) was added to this solution and stirred at room temperature for 4 hours. The mixture was neutralized with acetic acid/methanol. The mixture was concentrated under reduce pressure. The residue was purified by preparative HPLC to give the title compound 69 as a white solid (150 mg, 74%): LCMS: 506 [M+1]⁻; ¹H NMR (DMSO-d₆): 1.58 (m, 4H), 1.98 (t, J=5.7 Hz, 2H), 2.46 (t, 2H), 3.30 (s, 3H), 3.78 (t, J=4.2 Hz, 2H), 4.32 (t, J=5.1 Hz, 2H), 7.28 (s, 1H), 7.39 (t, J=9 Hz, 1H), 7.79 (m, 1H), 8.11 (m, J=2.7 Hz, 1H), 8.50 (s, 1H), 8.64 (s, 1H), 8.75 (s, 1H), 9.25 (s, 1H), 9.76 (s, 1H), 10.33 (s, 1H).

### Example 30

### Preparation of N¹-(4-(3-chloro-4-fluorophenylamino)-7-(2-methoxyethoxy)quinazolin-6-yl)-N⁸-hydroxyoctanediamide (Compound 70)

### Step 30a. Methyl 8-(4-(3-chloro-4-fluorophenylamino)-7-(2-methoxyethoxy)quinazolin-6-ylamino)-8-oxooctanoate (Compound 0310-70)

Methyl 8-chloro-8-oxooctanoate (0.496 g, 2.4 mmol) was added to a solution of compound 0308-68 (0.219 g, 0.6 mmol), 30 mL of dichloromethane and triethylamine (0.48 g, 2.4 mmol). The mixture was stirred for 2 hours at 0° C. The reaction was washed with water and dried over sodium sulfate, filtered and evaporated to give the title product 0310-70 as a brown oil (281 mg, 88%): LCMS: 533 [M+1]⁺. ¹H NMR (DMSO-d₆) 1.35 (m, 4H), 1.58 (m, 2H), 1.61 (m, 2H), 2.29 (t, J=7.2 Hz, 2H), 2.41 (t, J=7.2 Hz, 2H), 3.35 (s, 3H), 3.77 (t, J=4.5 Hz, 2H), 4.32 (t, J=4.5 Hz, 2H), 7.28 (s, 1H), 7.40 (t, J=9.3 Hz, 1H), 7.78 (m, 1H), 8.11 (m, 1H), 8.50 (s, 1H), 8.74 (s, 1H), 9.24 (s, 1H), 9.76 (s, 1H).

### Step 30b. N¹-(4-(3-chloro-4-fluorophenylamino)-7-(2-methoxyethoxy)quinazolin-6-yl)-N⁸-hydroxyoctanediamide (compound 70)

The freshly prepared hydroxylamine solution (6 mL, 4.0 mmol) was placed in 25 mL flask. Compound 0310-70 (281 mg, 0.53 mmol) was added to this solution and stirred at room temperature for 4 hours. The mixture was neutralized with acetic acid/methanol. The mixture was concentrated under reduce pressure. The residue was purified by preparative HPLC to give the title compound 70 as a yellow solid (126 mg, 40%): LCMS: 506 [M+1]; ¹H NMR (DMSO-d₆), 1.35 (m, 4H), 1.58 (m, J=6.9 Hz, 2H), 1.61 (m, J=7.2 Hz, 2H), 1.93 (t, J=7.2 Hz, 2H), 2.42 (t, J=7.5 Hz, 2H), 3.35 (s, 3H), 3.77 (t, J=4.5 Hz, 2H), 4.32 (t, J=4.5 Hz, 2H), 7.28 (s, 1H), 7.40 (t, J=9.3 Hz, 1H), 7.78 (m, 1H), 8.11 (m, J=2.4 Hz, 1H), 8.50 (s, 1H), 8.62 (d, J=1.5 Hz, 1H), 8.75 (s, 1H), 9.25 (s, 1H), 9.76 (s, 1H), 10.31 (s, 1H).

### Example 31

### Preparation of 7-(4-(3-ethynyl-4-fluorophenylamino)-7-methoxyquinazolin-6-yloxy)-N-hydroxyheptanamide (Compound 75)

### Step 31a. 2-Bromo-1-fluoro-4-nitrobenzene (compound 0602)

To a solution of 1-bromo-2-fluorobenzene (35.0 g, 200 mmol) in 200 mL of concentrated sulfuric acid was added 20 mL of 68% nitric acid. The temperature of the mixture was maintained below 20° C. After the addition was completed, the mixture was stirred at 10° C. overnight, then diluted with ice water. The resulting solid was collected by filtration. The solid was recrystallized from petroleum ether to give the title compound 0602 as a yellow solid (38 g, 89%): m.p. 55.8-56.7° C., ¹H NMR (DMSO-d₆): δ 7.66 (t, J=9 Hz, 1H), 8.32 (m, 1H), 8.58 (dd, J=3 Hz, 6 Hz, 1H).

### Step 31b. ((2-Fluoro-5-nitrophenyl)ethynyl)trimethylsilane (Compound 0603)

A mixture of compound 0602 (11.0 g, 50 mmol), ethynyltrimethylsilane (7.5 g, 75 mmol), triphenylphosphine (0.5 g) and palladium (II) acetate (0.25 g) in 125 mL of deaerated triethylamine was heated at 100° C. overnight under argon. The reaction was cooled and was filtrated, and the filtrate was concentrated to a dark brown oil which was distilled under reduce pressure to give title compound 0603 as a light brown solid (4.7 g, 40%). ¹H NMR (CDCl₃): δ 0.3 (s, 9H, SiCH), 7.22 (t, J=9.0 Hz, 1H), 8.2-8.5 (m, 2H).

### Step 31c. 4-Fluoro-3-((trimethylsilyl)ethynyl)benzenamine (Compound 0604)

In 25 mL of methanol was mixed with compound 0603 (3.5 g, 14.8 mmol) and iron filings (4.14 g, 74.0 mmol). To this mixture was added concentrated hydrochrolic acid and water to adjust pH 4-5. The mixture was heated to reflux for 3 hours, cooled, and filtrated through silica gel. The filtrate was concentrated to yield a yellow solid residue which was then extracted with ether. The combined organic phase was dried over magnesium sulfate and concentrated to give the title compound 0604 as a brown solid (2.69 g, 88%): LCMS 208 [M+1]⁺.

### Step 31d. 3-Ethynyl-4-fluorobenzenamine (Compound 0605)

Compound 0604 obtained above was treated with 100 mg potassium hydroxide in 20 mL of methanol at room temperature overnight. The solution was concentrated, dilute with water, brought to neutrality, and then extracted with ether. The combined organic phase was dried over magnesium sulfate, concentrated to yield the title compound 0605 as a brown oil (1.49 g, 85%): LCMS 136 [M+1]⁺. The product was used in the next step without further purification.

### Step 31e. 4-(3-Ethynyl-4-fluorophenylamino)-7-methoxyquinazolin-6-yl acetate (Compound 0606)

A mixture of 4-chloro-7-methoxyquinazolin-6-yl acetate (compound 0105) (252 mg, 1.0 mmol) and 3-ethynyl-4-fluorobenzenamine (605) (200 mg, 1.5 mmol) in isopropanol (10 mL) was stirred and heated to reflux for 3 hours. The mixture was cooled to room temperature and resulting precipitate was isolated. The solid was then dried to give the title compound 0606 (260 mg, 74.0%) as a light yellow solid: LCMS: 352 [M+1]⁺.

### Step 31f. 4-(3-Ethynyl-4-fluorophenylamino)-7-methoxyquinazolin-6-ol (Compound 0607)

A mixture of compound 0606 (260 mg, 0.74 mmol), LiOH H₂O (250 mg, 5.8 mmol) in methanol (25 ml) and H₂O (25 ml) was stirred at room temperature for 0.5 hour. The mixture was neutralized by addition of dilution acetic acid. The precipitate was isolated and dried to give the title compound 0607 (234 mg, 100%) as a grey solid: LCMS: 310 [M+1].

### Step 31g. Ethyl 7-(4-(3-ethynyl-4-fluorophenylamino)-7-methoxyquinazolin-6-yloxy)heptanoate (Compound 0608-75)

The title compound 0608-75 was prepared as a yellow solid (300 mg, 87.0%) from compound 607 (230 mg, 0.74 mmol) and ethyl 7-bromoheptanoate (176 mg, 0.74 mmol) using a procedure similar to that described for compound 0110-1 (Example 1): LCMS: 466 [M+1]⁺.

### Step 31h. 7-(4-(3-Ethynyl-4-fluorophenylamino)-7-methoxyquinazolin-6-yloxy)-N-hydroxyheptanamide (compound 75)

The title compound 75 was prepared as a white solid (176 mg, 70%) from compound 0608 (250 mg, 0.54 mmol) using a procedure similar to that described for compound 1 (Example 1): mp 150.4˜164.5° C. (dec); LCMS: 453 [M+1], ¹H NMR (DMSO-d₆): δ 1.33 (m, 2H), 1.48 (m, 4H), 1.80 (m, 2H), 1.94 (t, J=7.2 Hz, 2H), 3.91 (s, 3H), 4.10 (t, J=6.0 Hz, 2H), 4.51 (s, 1H), 7.17 (s, 1H), 7.31 (t, J=7.5 Hz, 1H), 7.77 (s, 1H), 7.85 (m, 1H), 7.98 (m, 1H), 8.45 (s, 1H), 8.65 (s, 1H), 9.47 (s, 1H), 10.33 (s, 1H).

### Example 32

### Preparation of (R)—N-hydroxy-6-(7-methoxy-4-(1-phenylethylamino)quinazolin-6-yloxy)-hexanamide (Compound 77)

### Step 32a. (R)-7-Methoxy-4-(1-phenylethylamino)quinazolin-6-ol (Compound 0701-77)

A mixture of compound 0105 (2.0 g, 8.0 mmol), (R)-1-phenylethanamine (2.91 g, 24.0 mmol) and isopropanol (50 mL) was stirred at 60° C. overnight. Iospropanol was removed and the residue was purified by column chromatography to give the title compound 0701-77 (1.32 g, 56%). LCMS: 296 [M+1]⁺.

### Step 32b. (R)-Ethyl 6-(7-methoxy-4-(1-phenylethylamino)quinazolin-6-yloxy)hexanoate (Compound 0702-77)

A mixture of compound 0701-77 (500.0 mg, 1.69 mmol), K₂CO₃ (700.0 mg, 5.07 mmol), ethyl 6-bromohexanoate (378.0 mg, 1.69 mmol) and DMF (20 mL) was heated at 60° C. for 3 h. The DMF was moved under reduced pressure, the residue was suspended in water, and the resulting solid was collected and dried to give the title compound 0702-77 (320 mg, 43%). LCMS: 438 [M+1]⁺.

### Step 32c. (R)—N-Hydroxy-6-(7-methoxy-4-(1-phenylethylamino)quinazolin-6-yloxy)-hexanamide (compound 77)

A mixture of compound 0702-77 (320.0 mg, 0.73 mmol) and 1.77 mol/L NH₂OH/MeOH (4.0 mL, 6.77 mmol) was stirred at room temperature for 0.5 h. The reaction mixture was neutralized with AcOH and concentrated. The residue was suspended in water and the resulting solid was isolated and dried to give crude product. This crude product was purified by pre-HPLC to give the title compound 77 (36 mg, 12%). LCMS: 425 [M+1]⁺; ¹H NMR (DMSO-d₆): δ 1.46 (m, 2H), 1.59 (m, 5H), 1.82 (m, 2H), 2.01 (t, J=8.7 Hz, 2H), 3.90 (s, 3H), 4.10 (t, J=6.3 Hz, 2H), 5.63 (m, 1H), 7.09 (s, 1H), 7.21 (m, 1H), 7.32 (m, 2H), 7.42 (d, J=7.2 Hz, 2H), 7.75 (s, 1H), 8.06 (d, J=8.4 Hz, 1H), 8.27 (s, 1H), 8.67 (s, 1H), 10.36 (s, 1H).

### Example 33

### Preparation of (R)—N-hydroxy-6-(4-(1-phenylethylamino)-quinazolin-6-yloxy)hexanamide (Compound 78)

### Step 33a. (R)-4-(1-Phenylethylamino)quinazolin-6-ol (Compound 0701-78)

A mixture of compound 0204 (1.0 g, 4.5 mmol) and (R)-1-(3-chloro-4-fluoro-phenyl)ethanamine (0.87 g, 5.0 mmol) in isopropanol (45 mL) was stirred at 90° C. for 1 hour. The mixture was cooled to room temperature and the resulting precipitate was isolated. The solid was washed in turn with isopropanol and methanol, dried to provide the title compound (R)-4-(1-phenylethylamino)quinazolin-6-yl acetate as a yellow solid (0.62 g, 61%): LCMS 308 [M+1]⁺.

A mixture of the above product (0.7 g, 2.3 mmol) and lithium hydroxide monohydrate (0.29 g, 6.81 mmol) in methanol (10 mL)/water (15 mL) was stirred at room temperature for 1 hour. The pH was adjusted to 4 with acetic acid and filtered. The collected yellow solid was washed by water and dried to obtained title compound 0701-78 as a yellow solid (0.42 g, 62%), LCMS 266 [M+1]⁺.

### Step 33b. (R)-Ethyl 6-(4-(1-phenylethylamino)quinazolin-6-yloxy)hexanoate (Compound 0702-78)

A mixture of compound 0701-78 (0.31 g, 1.2 mmol), ethyl 6-bromohexanoate (0.27 g, 1.2 mmol) and K₂CO₃ (0.8 g, 5.8 mmol) in DMF (15 mL) was stirred and heated to 80° C. for 2 hours. The mixture was filtered and the filtrate was evaporated. The resulting solid was washed with ether to obtain the title compound 0702-78 as a pale yellow solid (0.2 g, 42.5%), LCMS 408 [M+1]⁺.

### Step 33c. (R)—N-Hydroxy-6-(4-(1-phenylethylamino)quinazolin-6-yloxy)hexanamide (Compound 78)

The title compound 78 was prepared as a pale yellow solid (42 mg, 26%) from compound 0702-78 (168 mg, 0.41 mmol) using a procedure similar to that described for compound 77 (Example 32): LCMS 395 [M+1]⁺, ¹H NMR (DMSO-d₆): δ 1.47 (m, 2H), 1.52 (m, 2H) 1.65 (d, J=7.2 Hz, 3H) 1.71 (m, 2H), 2.05 (t, J=3.9 Hz, 2H), 4.04 (t, J=6.3 Hz, 2H), 5.56 (q, J=6.3 Hz, 1H) 7.13 (t, J=7.2 Hz, 1H), 7.26 (t, J=7.8 Hz, 2H), 7.32 (dd, J=2.7, J=9.0 Hz, 1H) 7.39 (d, J=7.2 Hz, 2H), 7.56 (d, J=7.2 Hz, 1H), 7.65 (m, 1H), 8.26 (s, 1H).

### Example 34

### Preparation of (R)—N-hydroxy-7-(7-methoxy-4-(1-phenylethylamino)quinazolin-6-yloxy)heptanamide (Compound 79)

### Step 34a. (R)-Ethyl 7-(7-methoxy-4-(1-phenylethylamino)quinazolin-6-yloxy)heptanoate (Compound 0702-79)

A mixture of compound 0701-79 (500 mg, 1.69 mmol), K₂CO₃ (700 mg, 5.07 mmol), ethyl 7-bromoheptanoate (401 mg, 1.69 mmol) and DMF (20 mL) was heated at 60° C. for 3 h. The DMF was removed under reduced pressure and the residue was suspended in water. The resulting solid was collected and dried to give the title compound 0702-79 (340 mg, 44%). LCMS: 452 [M+1].

### Step 34b. (R)—N-Hydroxy-7-(7-methoxy-4-(1-phenylethylamino)quinazolin-6-yloxy)heptanamide (Compound 79)

The title compound 79 was prepared (41 mg, 12%) from compound 0702-79 (340 mg, 0.75 mmol) using a procedure similar to that described for compound 77 (Example 32): LCMS: 439 [M+1]; ¹H NMR (DMSO-d₆): δ 1.34 (m, 2H), 1.52 (m, 4H), 1.58 (d, J=7.5 Hz, 2H), 1.80 (m, 2H), 1.99 (t, J=8.7 Hz, 2H), 3.89 (s, 3H), 4.10 (t, J=6.3 Hz, 2H), 5.62 (m, 1H), 7.08 (s, 1H), 7.20 (m, 1H), 7.31 (m, 2H), 7.41 (d, J=7.2 Hz, 2H), 7.74 (s, 1H), 8.05 (d, J=8.1 Hz, 1H), 8.26 (s, 1H), 8.63 (s, 1H), 10.32 (s, 1H).

### Example 35

### Preparation of (S)—N-hydroxy-7-(7-methoxy-4-(1-phenylethylamino)quinazolin-6-yloxy)heptanamide (Compound 80)

### Step 35a. (S)-7-Methoxy-4-(1-phenylethylamino)quinazolin-6-ol (Compound 0701-80)

The title compound 0701-80 was prepared as a yellow solid (556 mg, 62.8%) from compound 0105 (750 mg, 3.0 mmol) and (S)-1-phenylethanamine (1089 mg, 9.0 mmol) using a procedure similar to that described for compound 0701-77 (Example 32): LCMS: 296 [M+1]⁺.

### Step 35b. (S)-Ethyl 7-(7-methoxy-4-(1-phenylethylamino)quinazolin-6-yloxy)heptanoate (Compound 0702-80)

The title compound 0702-80 was prepared as a yellow solid (160 mg, 70.95%) from compound 701-80 (148 mg, 0.5 mmol) and ethyl 7-bromoheptanoate (120 mg, 0.5 mmol) using a procedure similar to that described for compound 0702-77 (Example 32): LCMS: 452 [M+1]⁺.

### Step 35c. (S)—N-hydroxy-7-(7-methoxy-4-(1-phenylethylamino)quinazolin-6-yloxy)heptanamide (Compound 80)

The title compound 80 was prepared as a white solid (95 mg, 61.9%) from compound 0702-80 (160 mg, 0.35 mmol) and fresh NH₂OH/CH₃OH (3 mL, 5.31 mmol) using a procedure similar to that described for compound 77 (Example 32): m.p. 106.7˜111.3° C., LCMS: 439 [M+1], ¹H NMR (DMSO-d₆): δ 1.42 (m, 6H), 1.57 (d, J=6.6 Hz, 3H), 1.79 (m, 2H), 1.95 (t, J=7.2 Hz, 2H), 3.88 (s, 3H), 4.08 (t, J=6.9 Hz, 2H), 5.62 (m, J=6.6 Hz, 1H), 7.06 (s, 1H), 7.21 (t, J=7.5 Hz, 1H), 7.30 (t, J=7.5 Hz, 2H), 7.41 (d, J=7.5 Hz, 2H), 7.75 (s, 1H), 8.15 (d, J=9.6 Hz, 1H), 8.29 (s, 1H), 8.60 (s, 1H), 10.30 (s, 1H).

### Example 36

### Preparation of (R)-7-(4-(1-(4-fluorophenyl)ethylamino)-7-methoxyquinazolin-6-yloxy)-N-hydroxyheptanamide (Compound 81)

### Step 36a. (R)-4-(1-(4-Fluorophenyl)ethylamino)-7-methoxyquinazolin-6-ol (Compound 0701-81)

The title compound 0701-81 was prepared as a yellow solid (495 mg, 52.71%) from compound 0105 (750 mg, 3.0 mmol) and (R)-1-(4-fluorophenyl)ethanamine (1251 mg, 9.0 mmol) using a procedure similar to that described for compound 0701-77 (Example 32): LCMS: 314 [M+1]⁺.

### Step 36b. (R)-Ethyl 7-(4-(1-(4-fluorophenyl)ethylamino)-7-methoxyquinazolin-6-yloxy)heptanoate (Compound 0702-81)

The title compound 0702-81 was prepared as a yellow solid (190 mg, 81.0%) from compound 0701-81 (156 mg, 0.5 mmol) and ethyl 7-bromoheptanoate (120 mg, 0.5 mmol) using a procedure similar to that described for compound 0702-77 (Example 32): LCMS: 470 [M+1]⁺.

### Step 36c. (R)-7-(4-(1-(4-Fluorophenyl)ethylamino)-7-methoxyquinazolin-6-yloxy)-N-hydroxyheptanamide (Compound 81)

The title compound 81 was prepared as a white solid (100 mg, 54.12%) from compound 0702-81 (190 mg, 0.40 mmol) and fresh NH₂OH/CH₃OH (3 mL, 5.31 mmol) using a procedure similar to that described for compound 77 (Example 32): m.p. 118.2-144.3° C., LCMS: 457 [M+1]⁺, ¹H NMR (DMSO-d₆): δ 1.33 (m, 2H), 1.47 (m, 4H), 1.56 (d, J=7.2 Hz, 3H), 1.78 (m, 2H), 1.95 (t, J=7.2 Hz, 2H), 3.87 (s, 1H), 4.07 (t, J=6.0 Hz, 2H), 5.60 (m, 1H), 7.06 (s, 1H), 7.11 (t, J=9.0 Hz, 2H), 7.44 (m, 2H), 7.71 (s, 1H), 8.04 (d, J=7.5 Hz, 1H), 8.25 (s, 1H), 8.65 (s, 1H), 10.33 (s, 1H).

### Example 37

### Preparation of (R)-7-(4-(1-(4-chlorophenyl)ethylamino)-7-methoxyquinazolin-6-yloxy)-N-hydroxyheptanamide (Compound 82)

### Step 37a. (R)-4-(1-(4-Chlorophenyl)ethylamino)-7-methoxyquinazolin-6-ol (Compound 0701-82)

The title compound 0701-82 was prepared as a yellow solid (0.65 g, 49%) from compound 0105 (1.0 g, 4 mmol) and (R)-1-(4-chlorophenyl)ethanamine (1.87 g, 12 mmol) using a procedure similar to that described for compound 0701-77 (Example 32): LCMS: 300 [M+1]⁺.

### Step 37b. (R)-Ethyl 7-(4-(1-(4-chlorophenyl)ethylamino)-7-methoxyquinazolin-6-yloxy)heptanoate (Compound 0702-82)

The title compound 0702-82 was prepared as a yellow solid (460 mg, 56%) from compound 0701-82 (550 mg, 1.7 mmol) and ethyl 7-bromoheptanoate (404 mg, 1.7 mmol) using a procedure similar to that described for compound 0702-77 (Example 32): LCMS: 486 [M+1]⁺.

### Step 37c. (R)-7-(4-(1-(4-Chlorophenyl)ethylamino)-7-methoxyquinazolin-6-yloxy)-N-hydroxyheptanamide (Compound 82)

The title compound 82 was prepared as a white solid (145 mg, 29%) from compound 0702-81 510 mg, 1.05 mmol) and fresh 0.77 mol/L NH₂OH/MeOH (4.7 mL, 8.4 mmol) using a procedure similar to that described for compound 77 (Example 32): LCMS: 473 [M+1]⁺; ¹H NMR (DMSO-d₆): δ 1.34 (m, 2H), 1.47 (m, 4H), 1.57 (d, J=6.9 Hz, 3H), 1.80 (m, 2H), 1.97 (t, J=7.2 Hz, 2H), 3.89 (s, 3H), 4.10 (t, J=6.6 Hz, 2H), 5.57 (m, 1H), 7.08 (s, 1H), 7.38 (d, J=8.4 Hz, 2H), 7.43 (d, J=8.4 Hz, 2H), 7.73 (s, 1H), 8.04 (d, J=7.8 Hz, 1H), 8.23 (s, 1H), 8.64 (s, 1H), 10.33 (s, 1H).

### Example 38

### Preparation of (R)—N-hydroxy-7-(7-methoxy-4-(1-(4-methoxyphenyl)ethylamino)quinazolin-6-yloxy)-heptanamide (Compound 83)

### Step 38a. (R)-7-Methoxy-4-(1-(4-methoxyphenyl)ethylamino)quinazolin-6-ol (Compound 0701-83)

A mixture of compound 0105 (1.0 g, 4.0 mmol), (R)-1-(4-methoxyphenyl)ethanamine (1.81 g, 12.0 mmol) and isopropanol (25 mL) was stirred at 60° C. overnight. Iospropanol was removed and the residue was purified by column chromatogram to give the title compound 0701-83 (0.81 g, 62%). LCMS: 326 [M+1]⁺.

### Step 38b. (R)-Ethyl 7-(7-methoxy-4-(1-(4-methoxyphenyl)ethylamino)quinazolin-6-yloxy)heptanoate (Compound 0702-83)

A mixture of compound 0701-83 (630 mg, 1.94 mmol), K₂CO₃ (804 mg, 5.8 mmol), ethyl 7-bromoheptanoate (459 mg, 1.94 mmol) and DMF (20 mL) was heated to 60° C. for 3 h. The DMF was moved away under reduced pressure, the residue was suspended in water, and the solid was collected and dried to give the title compound 0703-83 (440 mg, 47%). LCMS: 482 [M+1]⁺.

### Step 38c. (R)—N-Hydroxy-7-(7-methoxy-4-(1-(4-methoxyphenyl)ethylamino)-quinazolin-6-yloxy)-heptanamide (Compound 83)

A mixture of compound 0702-83 (530 mg, 1.1 mmol) and 1.77 mol/L NH₂OH/MeOH (5 mL, 8.8 mmol) was stirred at room temperature for 0.5 h. The reaction mixture was neutralized with AcOH and then the mixture was concentrated and the residue was suspended in water, the precipitate was isolated and dried to give crude product. This product was purified by pre-HPLC to give the title compound 83 (151 mg, 29%). LCMS: 469 [M+1]⁺; ¹H NMR (DMSO-d₆): δ 1.32 (m, 2H), 1.45 (m, 4H), 1.54 (d, J=6.9 Hz, 3H), 1.78 (m, 2H), 1.95 (t, J=7.2 Hz, 2H), 3.69 (s, 3H), 3.87 (s, 3H), 4.07 (t, J=6.3 Hz, 2H), 5.56 (m, 1H), 6.87 (d, J=8.7 Hz, 2H), 7.05 (s, 1H), 7.31 (d, J=8.7 Hz, 2H), 7.70 (s, 1H), 7.96 (d, J=8.1 Hz, 1H), 8.26 (s, 1H), 8.62 (s, 1H), 10.31 (s, 1H).

### Example 39

### Preparation of 7-(4-(Benzylamino)-7-methoxyquinazolin-6-yloxy)-N-hydroxyheptanamide (Compound 85)

### Step 39a. 4-(Benzylamino)-7-methoxyquinazolin-6-ol (0701-85)

Benzylamine (1.28 g, 12.0 mmol) was added into a mixture of compound 0105 (1.0 g, 4.0 mmol) and 2-propanol (50 ml). The reaction mixture was then stirred at reflux for 3 hours. The mixture was cooled to room temperature and the resulting precipitate was isolated. The solid was then dried to give the title compound 0701-85 as a yellow solid (854 mg, 76%): LCMS: 282 [M+1].

### Step 39b. Ethyl 7-(4-(benzylamino)-7-methoxyquinazolin-6-yloxy)heptanoate (Compound 0702-85)

The title compound 0702-85 was prepared as a yellow solid liquid (270 mg, 62%) from compound 0701-85 (281 mg, 1.0 mmol) and ethyl 7-bromoheptanoate (236 mg, 1 mmol) using a procedure similar to that described for compound 0702-77 (Example 32): LCMS: 438 [M+1]⁺.

### Step 39c. 7-(4-(Benzylamino)-7-methoxyquinazolin-6-yloxy)-N-hydroxyheptanamide (compound 85)

The title compound 85 was prepared as a yellow solid (64 mg, 24%) from compound 0702-85 (270 mg, 0.62 mmol) using a procedure similar to that described for compound 77 (Example 32): LCMS: 425 [M+1]⁺; ¹H NMR (DMSO-d₆): δ 1.32 (m, 2H), 1.42 (m, 2H), 1.51 (m, 2H), 1.76 (m, 2H), 1.94 (t, J=7.2 Hz, 2H), 3.88 (s, 3H), 4.03 (t, J=6.3 Hz, 2H), 4.76 (d, J=5.4 Hz, 2H), 7.08 (s, 1H), 7.21 (t, J=6.0 Hz, 2H), 7.30 (t, J=6.0 Hz, 2H), 7.33 (t, J=6.6 Hz, 1H), 7.63 (s, 1H), 8.29 (s, 1H), 8.42 (t, J=6.0 Hz, 1H), 8.64 (s, 1H), 10.32 (s, 1H).

### Example 40

### Preparation of 7-(4-(4-fluorobenzylamino)-7-methoxyquinazolin-6-yloxy)-N-hydroxyheptanamide (Compound 86)

### Step 40a. 4-(4-Fluorobenzylamino)-7-methoxyquinazolin-6-ol (Compound 0701-86)

The title compound 0701-86 was prepared as a yellow solid (489 mg, 54.5%) from compound 0105 (750 mg, 3.0 mmol) and (4-fluorophenyl)methanamine (1125 mg, 9.0 mmol) using a procedure similar to that described for compound 0701-77 (Example 32): LCMS: 300 [M+1]⁻.

### Step 40b. Ethyl 7-(4-(4-fluorobenzylamino)-7-methoxyquinazolin-6-yloxy)heptanoate (Compound 0702-86)

The title compound 0702-86 was prepared as a yellow liquid (408 mg, 89.67%) from compound 0701-86 (300 mg, 1.0 mmol), ethyl 7-bromoheptanoate (237 mg, 1.0 mmol) using a procedure similar to that described for compound 0702-77 (Example 32): LCMS: 456 [M+1]⁺.

### Step 40c. 7-(4-(4-fluorobenzylamino)-7-methoxyquinazolin-6-yloxy)-N-hydroxyheptanamide (Compound 86)

The title compound 86 was prepared as a white solid (300 mg, 69.97%) from compound 0702-86 (442 mg, 0.97 mmol) and fresh NH₂OH/CH₃OH (4 mL, 7.08 mmol) using a procedure similar to that described for compound 77 (Example 32): LCMS: 443 [M+1]⁺, ¹H NMR (DMSO-d₆): δ 1.31˜1.54 (m, 6H), 1.77 (m, 2H), 1.94 (t, J=7.5 Hz, 2H), 3.88 (s, 3H), 4.03 (t, J=6.3 Hz, 2H), 4.74 (d, J=5.4 Hz, 2H), 7.11 (m, 3H), 7.38 (m, 2H), 7.68 (s, 1H), 8.30 (s, 1H), 8.40 (m, 1H), 8.60 (s, 1H), 10.30 (s, 1H).

### Example 41

### Preparation of 7-(4-(3,4-difluorobenzylamino)-7-methoxyquinazolin-6-yloxy)-N-hydroxyheptanamide (Compound 87)

### Step 41a. 4-(3,4-Difluorobenzylamino)-7-methoxyquinazolin-6-ol (Compound 0701-87)

The title compound 0701-87 was prepared as a light yellow solid (500 mg, 52.6%) from compound 105 (750 mg, 3.0 mmol) and (3,4-difluorophenyl)methanamine (1072 mg, 7.5 mmol) using a procedure similar to that described for compound 0701-77 (Example 32): LCMS: 318 [M+1]⁺.

### Step 41b. Ethyl 7-(4-(3,4-difluorobenzylamino)-7-methoxy-4-a,5-dihydroquinazolin-6-yloxy)heptanoate (Compound 0702-87)

The title compound 0702-87 was prepared as a light yellow solid (205 mg, 86.7%) from compound 0701-87 (160 mg, 0.5 mmol), ethyl 7-bromoheptanoate (237 mg, 1.0 mmol) using a procedure similar to that described for compound 0702-77 (Example 32): LCMS: 474 [M+1]⁺.

### Step 41c. 7-(4-(3,4-difluorobenzylamino)-7-methoxyquinazolin-6-yloxy)-N-hydroxyheptanamide (Compound 87)

The title compound 87 was prepared as a white solid (75 mg, 44.5%) from compound 0702-87 (173 mg, 0.366 mmol) and fresh NH₂OH/CH₃OH (2 mL, 3.4 mmol) using a procedure similar to that described for compound 77 (Example 32): LCMS: 461 [M+1]⁺, ¹H NMR (DMSO-d₆): δ 1.30 (m, 2H), 1.50 (m, 4H), 1.77 (m, 2H), 1.94 (t, J=7.2 Hz, 2H), 3.88 (s, 1H), 4.03 (t, J=6.6 Hz, 2H), 4.72 (d, J=6.0 Hz, 2H), 7.08 (s, 1H), 7.19 (s, 1H), 7.35 (m, 2H), 7.61 (s, 1H), 8.30 (s, 1H), 8.46 (t, J=6.0 Hz, 1H), 8.64 (s, 1H), 10.32 (s, 1H).

### Example 42

### Preparation of 7-(4-(3-chloro-4-fluorobenzylamino)-7-methoxyquinazolin-6-yloxy)-N-hydroxyheptanamide (Compound 88)

### Step 42a. 4-(3-Chloro-4-fluorobenzylamino)-7-methoxyquinazolin-6-ol (Compound 0701-88)

The title compound 0701-88 was prepared as a light yellow solid (500 mg, 50.1%) from compound 0105 (750 mg, 3.0 mmol) and (3-chloro-4-fluorophenyl)methanamine (1435 mg, 9 mmol) using a procedure similar to that described for compound 0701-77 (Example 32): LCMS: 334 [M+1].

### Step 42b. Ethyl 7-(4-(3-chloro-4-fluorobenzylamino)-7-methoxyquinazolin-6-yloxy)heptanoate (Compound 0702-88)

The title compound 0702-88 was prepared as a yellow solid (306 mg, 92.02%) from compound 0701-88 (227 mg, 0.68 mmol), ethyl 7-bromoheptanoate (161 mg, 0.68 mmol) using a procedure similar to that described for compound 0702-77 (Example 32): LCMS: 490 [M+1]⁺.

### Step 42c. 7-(4-(3-Chloro-4-fluorobenzylamino)-7-methoxyquinazolin-6-yloxy)-hydroxyheptanamide (Compound 88)

The title compound 88 was prepared as a white solid (210 mg, 70.02%) from compound 0702-88 (306 mg, 0.63 mmol) and fresh NH₂OH/CH₃OH (3 mL, 5.3 μmol) using a procedure similar to that described for compound 77 (Example 32): m.p. 143.1° C. (decomp.), LCMS: 477 [M+1]⁺, ¹H NMR (DMSO-d₆): δ 1.31 (m, 2H), 1.48 (m, 4H), 1.77 (m, 2H), 1.94 (t, J=7.2 Hz, 2H), 3.89 (s, 3H), 4.04 (t, J=6.6 Hz, 2H), 4.74 (d, J=5.4 Hz, 2H), 7.09 (s, 1H), 7.35 (d, J=7.8 Hz, 2H), 7.54 (d, J=8.4 Hz, 1H), 7.63 (s, 1H), 8.35 (s, 1H), 8.58 (m, 1H), 8.65 (s, 1H), 10.33 (s, 1H), 11.92 (s, 1H).

### Example 43

### Preparation of 7-(4-(3-bromobenzylamino)-7-methoxyquinazolin-6-yloxy)-N-hydroxyheptanamide (Compound 89)

### Step 43a. 4-(3-Bromobenzylamino)-7-methoxyquinazolin-6-ol (Compound 0701-89)

The title compound 0701-89 was prepared as a yellow solid (543 mg, 50.2%) from compound 0105 (750 mg, 3.0 mmol) and (3-bromophenyl)methanamine (1674 mg, 9 mmol) using a procedure similar to that described for compound 0701-77 (Example 32): LCMS: 360 [M+1]⁺.

### Step 43b. Ethyl 7-(4-(3-bromobenzylamino)-7-methoxyquinazolin-6-yloxy)heptanoate (Compound 0702-89)

The title compound 0702-89 was prepared as a yellow solid (230 mg, 89.15%) from compound 0701-89 (180 mg, 0.5 mmol), ethyl 7-bromoheptanoate (120 mg, 0.5 mmol) using a procedure similar to that described for compound 0702-77 (Example 32): LCMS: 516 [M+1].

### Step 43c. 7-(4-(3-bromobenzylamino)-7-methoxyquinazolin-6-yloxy)-N-hydroxyheptanamide (Compound 89)

The title compound 89 was prepared as a white solid (105 mg, 53.96%) from compound 0702-89 (200 mg, 0.39 mmol) and fresh NH₂OH/CH₃OH (3 mL, 5.31 mmol) using a procedure similar to that described for compound 77 (Example 32): LCMS: 503 [M+1]⁻, ¹H NMR (DMSO-d₆): δ 1.31˜1.56 (m, 6H), 1.75 (m, 2H), 1.94 (t, J=7.2 Hz, 2H), 3.88 (s, 3H), 4.06 (t, J=6.6 Hz, 2H), 4.75 (d, J=5.7 Hz, 2H), 7.08 (s, 1H), 7.27 (t, J=7.5 Hz, 1H), 3.7.33 (m, 2H), 7.42 (s, 1H), 7.61 (s, 1H), 7.93 (s, 1H), 8.30 (s, 1H), 8.41 (t, J=6.0 Hz, 1H), 8.60 (s, 1H), 10.29 (s, 1H).

### Example 44

### Preparation of 4-(2-(4-(3-chloro-4-fluorophenylamino)-7-methoxyquinazolin-6-yloxy)ethoxy)-N-hydroxybenzamide (Compound 92)

### Step 44a. Methyl 4-(2-bromoethoxy)benzoate (Compound 0502-92)

A mixture of compound 4-hydroxybenzoic acid methyl ester (457.0 mg, 3.0 mmol), K₂CO₃ (828 mg, 6 mmol) and 1,2-dibromoethane (10 mL) was heated at 130° C. for 8 h. The 1,2-dibromoethane was removed under reduced pressure and the residue was suspended in water. The resulting precipitate was isolated and dried to give the title compound 0502-92 as a white solid (440 mg, 57%). LCMS: 259 [M+1].

### Step 44b. Methyl 4-(2-(4-(3-chloro-4-fluorophenylamino)-7-methoxyquinazolin-6-yloxy)ethoxy)benzoate (Compound 0503-92)

A mixture of compound 109 (384 mg, 1.2 mmol), K₂CO₃ (276 mg, 2 mmol), compound 0502-92 (311 mg, 1.2 mmol) and DMF (10 mL) was heated at 40° C. overnight. The DMF was removed under reduced pressure and the residue was suspended in water. The precipitate was collected and dried to give the title compound 0503-92 as a white solid (430 mg, 72%). LCMS: 259 [M+1]⁺.

### Step 44c. 4-(2-(4-(3-Chloro-4-fluorophenylamino)-7-methoxyquinazolin-6-yloxy)ethoxy)-N-hydroxybenzamide (Compound 92)

A mixture of compound 0502-92 (249 mg, 0.5 mmol) and 1.77 mol/L NH₂OH/MeOH (5 mL, 8.85 mmol) was stirred at room temperature for 0.5 h. The reaction mixture was neutralized with AcOH and the mixture was concentrated and the residue was suspended in water. The resulting precipitate was isolated and dried to give crude product. This crude product was purified by pre-HPLC to give the title compound 92 as a white solid (80 mg, 32%). LCMS: 439 [M+1]⁺; ¹H NMR (DMSO-d₆): δ 2.07 (s, 2H), 3.93 (s, 3H), 4.50 (s, 4H), 7.08 (d, J=8.4 Hz, 2H), 7.22 (s, 2H), 7.44 (t, J=9.0 Hz, 1H), 7.76 (m, 3H), 7.89 (s, 1H), 8.12 (m, 1H), 8.51 (s, 1H), 8.87 (s, 1H), 9.54 (s, 1H), 11.05 (s, 1H).

### Example 45

### Preparation of 7-(4-(3-ethynylphenylamino)-7-methoxyquinazolin-6-yloxy)-N-methoxyheptanamide (Compound 95)

A mixture of compound 0802 (544 mg, 1.25 mmol) and Inodomethane (0804) (177 mg, 1.25 mmol) and potassium carbonate (1.0 g, 7.25 mmol) in N,N-dimethylformamide (15 mL) was stirred at room temperature for 12 hours. The solvent was removed under reduce pressure and the residue was dissolved in ethyl acetate (50 mL). The organic layer was washed with saturation aqueous NaHCO₃ (20 mL) and brine (20 mL). The organic layer was dried over MgSO₄ and concentrated to give the title compound 95 as pale yellow solid (500 mg, 89%). m.p. 195.8˜197.0° C.; LCMS: 449 [M+1]⁺; ¹H NMR (DMSO-d₆); δ 1.35 (m, 2H), 1.50 (m, 4H), 1.80 (m, 2H), 1.94 (t, J=7.2 Hz, 2H), 3.54 (s, 3H), 3.92 (s, 3H), 4.12 (t, J=6.3 Hz, 2H), 4.19 (s, 1H), 7.19 (m, 2H), 7.40 (t, J=7.8 Hz, 1H), 7.80 (s, 1H), 7.87 (d, J=9.6 Hz, 1H), 7.97 (s, 1H), 8.48 (s, 1H), 9.45 (s, 1H), 10.92 (s, 1H).

### Example 46

### Preparation of N-acetoxy-7-(4-(3-chloro-4-fluorophenylamino)-7-methoxyquinazolin-6-yloxy)heptanamide (Compound 96)

A mixture of compound 0801 (50 mg, 0.108 mmol) and Ac₂O (204 mg, 2.0 mmol) and AcOH (2 mL) was stirred at room temperature for 1 h. The reaction mixture was neutralized with NaHCO₃ saturation solution. The precipitate was isolated and dried to give product 96 (42 mg, 77%). LCMS: 505 [M+1]⁺; ¹H NMR (DMSO-d₆): δ 1.40 (m, 2H), 1.50 (m, 2H), 1.55 (m, 2H), 1.80 (m, 2H), 2.09 (s, 3H), 2.12 (m, 2H), 3.94 (s, 3H), 4.13 (t, J=6.9 Hz, 2H), 7.20 (s, 1H), 7.43 (t, J=9.0 Hz, 1H), 7.78 (m, 1H), 7.84 (s, 1H), 8.12 (m, 1H), 8.49 (s, 1H), 9.67 (s, 1H).

### Example 47

### Preparation of N-acetoxy-7-(4-(3-ethynylphenylamino)-7-methoxyquinazolin-6-yloxy)heptanamide (Compound 97)

The title compound 97 was prepared as a solid (45 mg, 86.0%) from compound 0802 (48 mg, 0.11 mmol) and Ac₂O (204 mg, 2 mmol) using a procedure similar to that described for compound 96 (Example 46): LCMS: 476.5 [M+1]⁺; ¹H NMR (DMSO-d₆): δ 1.40 (m, 2H), 1.46 (m, 2H), 1.58 (m, 2H), 1.80 (m, 2H), 2.12 (s, 3H), 2.13 (m, 2H), 3.94 (s, 3H), 4.14 (t, J=6.6 Hz, 2H), 4.19 (s, 1H), 7.20 (d, J=6.3 Hz, 2H), 7.40 (t, J=7.8 Hz, 1H), 7.83 (s, 1H), 7.89 (d, J=7.8 Hz, 1H), 7.99 (s, 1H), 8.49 (s, 1H), 9.50 (s, 1H), 11.55 (s, 1H).

### Example 48

### Preparation of N-(cyclohexanecarbonyloxy)-7-(4-(3-ethynylphenylamino)-7-methoxyquinazolin-6-yloxy)heptanamide (Compound 98)

Compound 0802 (218 mg, 0.5 mmol) and triethylamine (75 mg, 0.75 mmol) were dissolved in acetone (20 mL) and N,N-dimethylformamide (2 mL). The reaction mixture was cooled to 0° C. and a solution of cyclohexanecarbonyl chloride (73 mg, 0.5 mmol) in acetone (5 mL) was added into the above solution dropwise. The reaction mixture was allowed to raise to ambient temperature and stirred for 1 hour. The mixture was concentrated under reduce pressure and the residue was purified by column chromatography to give the title compound 98 as a yellow solid (50 mg, 18%): LCMS: 545 [M+1]⁺; ¹H NMR (DMSO-d₆): δ 1.21˜1.63 (m, 15H), 1.81 (m, 4H), 2.11 (t, J=7.2 Hz, 2H), 3.92 (s, 3H), 4.12 (t, J=7.2 Hz, 2H), 4.17 (s, 1H), 7.19 (m, 2H), 7.39 (t, J=7.8 Hz, 1H), 7.81 (s, 1H), 7.88 (d, J=8.4 Hz, 1H), 7.97 (s, 1H), 8.47 (s, 1H), 9.45 (s, 1H), 11.50 (s, 1H).

### Example 49

### Preparation of 7-(4-(3-ethynylphenylamino)-7-methoxyquinazolin-6-yloxy)-N-(isobutyryloxy)heptanamide (Compound 99)

The title compound 99 was prepared as a yellow solid (100 mg, 44.0%) from compound 0802 (195 mg, 0.45 mmol) and isobutyryl chloride (48 mg, 0.45 mmol) using a procedure similar to that described for compound 98 (Example 48): LCMS: 505 [M+1]⁻; ¹H NMR (DMSO-d₆): δ 1.10 (d, J=7.2 Hz, 6H), 1.39 (m, 2H), 1.47 (m, 2H), 1.56 (m, 2H), 1.81 (m, 2H), 2.11 (t, J=7.5 Hz, 2H), 2.68 (m, J=7.2 Hz, 2H), 3.92 (s, 3H), 4.12 (t, J=6.6 Hz, 2H), 4.17 (s, 1H), 7.19 (m, 2H), 7.38 (t, J=7.8 Hz, 1H), 7.82 (s, 1H), 7.88 (d, J=8.7 Hz, 1H), 7.97 (s, 1H), 8.47 (s, 1H), 9.50 (s, 1H), 11.55 (s, 1H).

### Example 50

### Preparation of 7-(4-(3-ethynylphenylamino)-7-methoxyquinazolin-6-yloxy)-N-(propionyloxy)heptanamide (Compound 100)

The title compound 100 was prepared as a yellow solid (100 mg, 41.0%) from compound 0802 (218 mg, 0.5 mmol) and propionyl chloride (47 mg, 0.5 mmol) using a procedure similar to that described for compound 98 (Example 48): LCMS: 491 [M+1]⁻; ¹H NMR (DMSO-d₆): δ 1.05 (t, J=7.5 Hz, 3H), 1.39 (m, 2H), 1.48 (m, 2H), 1.56 (m, 2H), 1.81 (m, 2H), 2.12 (t, J=6.6 Hz, 2H), 2.41 (q, J=7.5 Hz, 2H), 3.92 (s, 3H), 4.12 (t, J=6.6 Hz, 2H), 4.18 (s, 1H), 7.19 (m, 2H), 7.38 (t, J=7.8 Hz, 1H), 7.80 (s, 1H), 7.88 (d, J=8.1 Hz, 1H), 7.97 (s, 1H), 8.47 (s, 1H), 9.45 (s, 1H), 11.53 (s, 1H).

### Example 51

### Preparation of N-(benzoyloxy)-7-(4-(3-ethynylphenylamino)-7-methoxyquinazolin-6-yloxy)heptanamide (Compound 101)

The title compound 101 was prepared as a yellow solid (150 mg, 56.0%) from compound 0802 (218 mg, 0.5 mmol) and benzoyl chloride (72 mg, 0.5 mmol) using a procedure similar to that described for compound 98 (Example 48): LCMS: 539 [M+1]⁻; ¹H NMR (DMSO-d₆): δ 1.51 (m, 4H), 1.61 (m, 2H), 1.84 (m, 2H), 2.21 (t, J=7.5 Hz, 2H), 3.93 (s, 3H), 4.14 (t, J=6.9 Hz, 2H), 4.19 (s, 1H), 7.19 (m, 2H), 7.38 (t, J=7.8 Hz, 1H), 7.55 (m, 2H), 7.72 (t, J=7.8 Hz, 1H), 7.82 (s, 1H), 7.89 (d, J=8.7 Hz, 1H), 7.99 (m, 3H), 8.48 (s, 1H), 9.48 (s, 1H), 11.88 (s, 1H).

### Example 52

### Preparation of 7-(4-(3-ethynylbenzylamino)-7-methoxyquinazolin-6-yloxy)-N-hydroxyheptanamide (Compound 90)

### Step 52a. 4-(3-Ethynylbenzylamino)-7-methoxyquinazolin-6-ol (Compound 0701-90)

The title compound 701-90 was prepared as a light yellow solid (406 mg, 65%) from compound 105 (520 mg, 2.06 mmol) and 3-ethynylbenzylamine (600 mg, 4.6 mmol) in isopropanol (20 mL) using a procedure similar to that described for compound 701-77 (Example 32): LCMS: 306 [M+1]⁺.

### Step 52b. Ethyl 7-(4-(3-ethynylbenzylamino)-7-methoxyquinazolin-6-yloxy)heptanoate (Compound 0702-90)

The title compound 0702-90 was prepared as a yellow solid (350 mg, 57%) from compound 0701-90 (406 mg, 1.33 mmol), potassium carbonate and ethyl 7-bromoheptanoate using a procedure similar to that described for compound 0702-77 (Example 32): LCMS: 462 [M+1].

### Step 52c. 7-(4-(3-ethynylbenzylamino)-7-methoxyquinazolin-6-yloxy)-N-hydroxyheptanamide (Compound 90)

The title compound 90 was prepared as a white solid (30 mg, 8.8%) from compound 0702-90 (350 mg, 0.76 mmol) and fresh NH₂OH/CH₃OH (2 mL, 3.54 mmol) using a procedure similar to that described for compound 77 (Example 32): LCMS: 449 [M+1]⁻, ¹H NMR (DMSO-d₆): δ 1.30-1.53 (m, 6H), 1.74-1.78 (m, 2H), 1.92-1.96 (m, 2H), 3.88 (s, 3H), 4.04 (t, J=6.6 Hz, 2H), 4.11 (s, 1H), 4.75 (d, J=4.5 Hz, 2H), 7.08 (s, 1H), 7.33-7.37 (m, 3H), 7.43 (s, 1H), 7.61 (s, 1H), 8.30 (s, 1H), 8.41 (t, J=6.6 Hz, 1H), 8.60 (s, 1H), 10.29 (s, 1H).

### Example 53

### Preparation of N-(6-(4-(3-chloro-4-fluorophenylamino)-7-methoxyquinazolin-6-yloxy)hexyl)-N-hydroxyacetamide (Compound 103)

### Step 53a. 6-(4-(3-Chloro-4-fluorophenylamino)-7-methoxyquinazolin-6-yloxy)hexan-1-ol (Compound 0901)

A mixture of compound 0109 (1.1 g, 3.44 mmol) and K₂CO₃ (1.9 g, 13.76 mmol) in DMF (20 mL) was stirred at 40° C. for 10 min. 6-Bromohexan-1-ol (0.64 g, 3.44 mmol) was added and the mixture was stirred at 60° C. for 6 h. DMF was removed under reduced pressure and the residue was suspended in water. The resulting solid was collected and dried to give product 0901 (1.35 g, 93%). LC-MS: 420 [M+1]⁺.

### Step 53b: N-acetoxyacetamide (Compound 0902-103)

A mixture of hydroxylamine chloride (1.39 g, 20 mmol), sodium acetate (2.46 g, 30 mmol) and acetic anhydride (20.4 g, 200 mmol) in acetic acid (40 mL) was heated at reflux for 48 h. The reaction mixture was filtrated and concentrated. The residue was added with water (20 mL) and extracted with ethyl acetate (30 mL×3). The organic layer was collected, washed with saturated NaHCO₃ solution, brine, dried (MgSO₄), filtered and concentrated to give compound 0902-103 as a yellow liquid (2.11 g, 90%).

### Step 53c. N-Acetoxy-N-(6-(4-(3-chloro-4-fluorophenylamino)-7-methoxyquinazolin-6-yloxy)hexyl)acetamide (Compound 0903-103)

A mixture of compound 0902-103 (117 mg, 1.0 mmol), compound 0901 (210 mg, 0.5 mmol) and PPh₃ (524 mg, 2.0 mmol) were dissolved in dry THF (50 mL). The reaction mixture was stirred at room temperature and was then added (E)-diisopropyl diazene-1,2-dicarboxylate (404 mg, 2.0 mmol) slowly. The mixture was heated to reflux for 1 hour and concentrated. The residue (4.53 g) was purified by flash column chromatography on silica gel with petroleum ether:ethyl acetate=1:1 as eluant to give compound 0903-103 as a yellow solid (50 mg, 19%).

### Step 53d. N-(6-(4-(3-Chloro-4-fluorophenylamino)-7-methoxyquinazolin-6-yloxy)hexyl)-N-hydroxyacetamide (Compound 103)

A mixture of compound 0903 (50 mg, 0.1 mmol) in methanol (2 mL) and water (2 mL) was added LiOH.H₂O (6 mg, 0.15 mmol). The reaction mixture was stirred at room temperature for 30 minutes and was neutralized by acetate acid. The mixture was evaporated to remove methanol. The resulting solid was filtrated, washed with water, diethyl ether to give the title compound 103 as an orange solid (32 mg, 70%). LCMS: 477 [M+1]⁺; ¹H NMR (DMSO-d₆): δ 1.31 (m, 2H), 1.50 (m, 4H), 1.82 (m, 2H), 1.94 (s, 3H), 3.46 (t, J=7.2 Hz, 2H), 3.97 (s, 3H), 4.14 (t, J=6.3 Hz, 2H), 7.28 (s, 1H), 7.54 (t, J=9.0 Hz, 1H), 7.70 (m, 1H), 8.03 (dd, 1H), 8.16 (s, 1H), 8.82 (s, 1H), 9.70 (s, 1H).

### Example 54

### Preparation of N-(6-(4-(3-chloro-4-fluorophenylamino)-7-methoxyquinazolin-6-yloxy)hexyl)-N-hydroxypropionamide (Compound 106)

### Step 54a: N-(propionyloxy)propionamide (Compound 0902-106)

Hydroxylamine chloride (1.39 g, 20 mmol) was dissolved in DMF (20 mL) and acetone (20 mL). The reaction was cooled to −10° C. with ice/salt bath. To this cold solution was added Et₃N (20 mL, 120 mmol) and then propionyl chloride (7.4 g, 80 mmol) slowly. After addition, the mixture was warmed to room temperature and stirred for 1 h. Water (50 mL) was added to the reaction mixture and extracted with ethyl acetate (100 mL×3). The organic layer was collected, washed by saturated NaHCO₃ solution (20 mL×2) and brine (20 mL), dried (MgSO₄), filtered and concentrated to give the title product 0902-106 as an orange liquid (3.93 g, 100%): LCMS: 146 [M+1]⁺.

### Step 54b: 7-(4-(3-Chloro-4-fluorophenylamino)-7-methoxyquinazolin-6-yloxy)-N-hydroxy-N-methylheptanamide (Compound 0903-106)

To a mixture of compound 0902-106 (795 mg, 5.5 mmol), compound 0901-106 (419 mg, 1.0 mmol) and PPh₃ (1.31 g, 5.0 mmol) in dry THF (40 mL) was added (E)-diisopropyl diazene-1,2-dicarboxylate (1.01 g, 5 mmol) slowly at room temperature. The mixture was heated to reflux for 1 h and then concentrated to yield crude product 0903-106 (4.53 g) which was used to next step without further purifying.

### Step 54c: N-(6-(4-(3-Chloro-4-fluorophenylamino)-7-methoxyquinazolin-6-yloxy)hexyl)-N-hydroxypropionamide (Compound 106)

To compound 0903-106 (4.53 g crude) was added NH₃/EtOH solution (20 mL) in ice/water bath temperature. The reaction was then warmed to room temperature and stirred at room temperature over night. The reaction was filtered and the filtrate was concentrated to a residue which was purified by flash column chromatography on silica gel with ethyl acetate/petroleum ether (1:1) as eluant to give the title compound 106 as a white solid (89 mg, two steps total yield 19%): m.p. 149.2˜158.0° C. (dec); LCMS: 491 [M+1]⁻; ¹H NMR (DMSO-d₆): δ 0.92 (t, J=7.5 Hz, 3H), 1.33 (m, 2H), 1.50 (m, 4H), 1.81 (m, 2H), 2.29 (m, 2H), 3.46 (t, J=7.2 Hz, 2H), 3.90 (s, 3H), 4.09 (t, J=5.4 Hz, 2H), 7.16 (s, 1H), 7.41 (t, J=9.0 Hz, 1H), 7.76 (s, 1H), 7.78 (s, 1H), 8.07 (dd, 1H), 8.46 (s, 1H), 9.51 (s, 1H), 9.53 (s, 1H).

### Example 55

### Preparation of (R)—N-hydroxy-5-(5-(7-methoxy-4-(1-phenylethylamino)quinazolin-6-yl)furan-2-yl)pent-4-ynamide (Compound 124)

### Step 55a. (R)-6-iodo-7-methoxy-N-(1-phenylethyl)quinazolin-4-amine (Compound 1001)

A mixture of conc. H₂SO₄ (7.1 g), acetonitrile (96 mL), acetic acid (96 mL) and water (96 mL) containing compound 0308 (3.0 g, 9.4 mmol) was cooled to 0° C. and stirred for 0.5 h. The reaction mixture became a clear solution. To this solution was added NaNO₂ (0.72 g, 10.4 mmol) at 0° C. The resulting solution was stirred at room temperature for 0.5 hours and was then added dropwise to a solution of KI (4.68 g, 28 mmol) in water (96 mL) at 50° C. After the addition was completed, the resulting solution was stirred at 50° C. for another 0.5 hours. The reaction mixture was then cooled and filtered, washed with water and dried to give product 1001 as a yellow solid (2.5 g, 50% yield). ¹H NMR (d₆-DMSO) δ 10.12 (s, 1H), 9.16 (s, 1H), 8.92 (s, 1H), 8.00 (dd, J₁, J₂=6.9 Hz, 2.4 Hz, 1H), 7.66-7.70 (m, 1H), 7.54 (t, J=9.0 Hz, 1H), 7.20 (s, 1H). LC-MS: 406 (M+1).

### Step 55b. (R)-6-(Furan-2-yl)-7-methoxy-N-(1-phenylethyl)quinazolin-4-amine (Compound 1002)

A mixture of 1001 (4.29 g, 10 mmol), 2-furanbornic acid (2.2 g, 20 mmol), Pd(OAc)₂ (224 mg, 1.0 mmol), PPh₃ (524 mg, 2.0 mmol), triethylamine (10 mL) and dimethylformamide (30 mL) was stirred at 80° C. for 16 hours. The reaction mixture was cooled to room temperature and water (150 mL) was added. The resulting mixture was extracted with ethyl acetate (120 mL×4), dried and evaporated. The residue was purified by column chromatography (ethyl acetate: petroleum ether=1:3) to yield the product 1002 as a white solid (2.5 g, 67% yield). ¹H NMR (DMSO-d₆) δ10.01 (s, 1H), 8.83 (s, 1H), 8.53 (s, 1H), 8.16-8.17 (m, 1H), 7.84-7.86 (m, 2H), 7.41 (t, J=8.1 Hz, 1H), 7.29 (s, 1H), 7.06 (s, 1H), 6.66 (s, 1H), 4.05 (s, 3H). LC-MS: 370 (M+1).

### Step 55c. (R)-6-(Furan-2-yl)-7-methoxy-N-(1-phenylethyl)quinazolin-4-amine (Compound 1003)

To a solution of 1002 (1.48 g, 4 mmol) in trifluroacetic acid (2 mL) and acetonitrile (40 mL) was added NIS (650 mg, 5 mmol). The solution was stirred at room temperature for 10 min. The mixture was neutralized with aqueous Na₂CO₃ and concentrated. The resulting mixture was extracted with ethyl acetate, washed with water, dried, and concentrated to give a residue which was purified by column chromatography to afford 1003 as a yellow solid (1.1 g, 58% yield). ¹H NMR (DMSO-d₆) δ 10.08 (s, 1H), 8.72 (s, 1H), 8.55 (s, 1H), 8.15 (dd, J₁, J₂=6.9 Hz, 2.7 Hz, 1H), 7.79-7.83 (m, 1H), 7.45 (t, J=9.0 Hz, 1H), 7.31 (s, 1H), 7.00 (d, J=3.6 Hz, 1H), 6.91 (d, J=3.6 Hz, 1H), 4.06 (s, 3H). LC-MS: 496 (M+1).

### Step 55d. (R)-Methyl 5-(5-(7-methoxy-4-(1-phenylethylamino)quinazolin-6-yl)furan-2-yl)ynoate (Compound 1004-124)

A mixture of 1003 (250 mg, 0.5 mmol), methyl pent-4-ynoate (112 mg, 1.0 mmol), Pd(OAc)₂ (35 mg, 0.05 mmol), PPh₃ (13 mg, 0.05 mmol), CuI (10 mg, 0.05 mmol), Et₃N (0.5 mL) and DMF (3 mL) was stirred at 40° C. under nitrogen for 16 h. The mixture was then diluted with water (120 mL) and extracted with ethyl acetate (100 mL×4). The combined organic layer was concentrated and purified by column chromatography (ethyl acetate:petroleum ether=1:4) to afford 1004-124 as a yellow solid (180 mg, 78% yield). LC-MS: 480 (M+1).

### Step 55e. (R)—N-Hydroxy-5-(5-(7-methoxy-4-(1-phenylethylamino)quinazolin-6-yl)furan-2-yl)pent-4-ynamide (Compound 124)

To a flask containing compound 1004-124 (180 mg, 0.37 mmol) was added a solution of hydroxylamine in methanol (3.0 mL). The mixture was stirred at room temperature for 0.5 h. Then it was adjusted to PH 7 using acetic acid. The mixture was filtered, washed with methanol to afford the product 124 as a white solid (100 mg, 55% yield). ¹H NMR (DMSO-d₆) δ 10.52 (s, 1H), 10.13 (s, 1H), 8.85 (s, 1H), 8.79 (s, 1H), 8.53 (s, 1H), 8.12-8.16 (m, 1H), 7.79-7.83 (m, 1H), 7.43 (t, J=9.6 Hz, 1H), 7.30 (s, 1H), 7.09 (d, J=3.6 Hz, 1H), 6.85 (d, J=3.6 Hz, 1H), 4.05 (s, 3H), 2.73 (t, J=7.2 Hz, 2H), 2.26 (t, J=7.2 Hz, 2H). LC-MS: 481 (M+1).

### Example 56

### Preparation of (R)—N-hydroxy-6-(5-(7-methoxy-4-(1-phenylethylamino)quinazolin-6-yl)furan-2-yl)hex-5-ynamide (Compound 125)

### Step 56a. (R)-Methyl 6-(5-(7-methoxy-4-(1-phenylethylamino)quinazolin-6-yl)furan-2-yl)hex-5-ynoate (Compound 1004-125)

The title compound 1004-125 was prepared as a yellow solid (180 mg, 77%) from compound 1003 (250 mg, 0.5 mmol) and methyl hex-5-ynoate (126 mg, 1.0 mmol) using a procedure similar to that described for compound 1004-124 (Example 55): LCMS: 494 [M+1]⁺.

### Step 56b. (R)—N-Hydroxy-6-(5-(7-methoxy-4-(1-phenylethylamino)quinazolin-6-yl)furan-2-yl)hex-5-ynamide (Compound 125)

The title compound 125 was prepared as a white solid (60 mg, 13%) from compound 1004-125 (160 mg, 0.34 mmol) and hydroxylamine in methanol (3.0 mL) using a procedure similar to that described for compound 124 (Example 55): ¹H NMR (DMSO-d₆) δ 10.43 (s, 1H), 10.11 (s, 1H), 8.79 (s, 1H), 8.73 (s, 1H), 8.53 (s, 1H), 8.12-8.15 (m, 1H), 7.77-7.82 (m, 1H), 7.43 (t, J=9.0 Hz, 1H), 7.30 (s, 1H), 7.09 (d, J=3.6 Hz, 1H), 6.86 (d, J=3.6 Hz, 1H), 4.05 (s, 3H), 2.52 (t, J=6.6 Hz, 2H), 2.10 (t, J=7.2 Hz, 2H), 1.72-1.82 (m, 2H). LC-MS: 495 (M+1).

### Example 57

### Preparation of methyl 5-(4-(3-chloro-4-fluorophenylamino)-7-methoxyquinazolin-6-yl)pent-4-ynoate (Compound 138)

### Step 57a. Methyl 5-(4-(3-chloro-4-fluorophenylamino)-7-methoxyquinazolin-6-yl)pent-4-ynoate (Compound 1101-138)

A mixture of 1001 (215 mg, 0.5 mmol), methyl pent-4-ynoate (224 mg, 2.0 mmol), Pd(OAc)₂ (140 mg, 0.2 mmol), PPh₃ (52 mg, 0.2 mmol), CuI (76 mg, 0.4 mmol), Et₃N (2.5 mL) and DMF (5 mL) was stirred at 80° C. for 16 h. Water (120 mL) was added to the reaction and the resulting mixture was extracted with ethyl acetate. The organic phase was combined, dried, filtered and concentrated to leave a residue which was purified by column chromatography (ethyl acetate:petroleum ether=1:4) to afford 1101 as a yellow solid (160 g, 77% yield). LC-MS: 414 (M+1).

### Step 57b. Methyl 5-(4-(3-chloro-4-fluorophenylamino)-7-methoxyquinazolin-6-yl)pent-4-ynoate (Compound 138)

To a flask containing compound 1101-138 (102 mg, 0.25 mmol) was added freshly prepared solution of hydroxylamine in methanol (3.0 mL). The mixture was stirred at room temperature for 0.5 h. It was then adjusted to PH 7 using acetic acid. The resulting precipitate was filtered and washed with methanol to afford the product 138 as a white solid (75 mg, 74% yield). ¹H NMR (DMSO-d₆) δ 10.49 (s, 1H), 9.81 (s, 1H), 8.81 (s, 1H), 8.56 (s, 1H), 8.55 (s, 1H), 8.17-8.20 (m, 1H), 7.79-7.84 (m, 1H), 7.42 (t, J=9.0 Hz, 1H), 7.19 (s, 1H), 3.94 (s, 3H), 2.72 (t, J=7.2 Hz, 2H), 2.28 (t, J=7.2 Hz, 2H). LC-MS: 415 (M+1).

### Example 58

### Preparation 6-(4-(3-chloro-4-fluorophenylamino)-7-methoxyquinazolin-6-yl)-N-hydroxyhex-5-ynamide (Compound 139)

### Step 58a. Methyl 6-(4-(3-chloro-4-fluorophenylamino)-7-methoxyquinazolin-6-yl)hex-5-ynoate (Compound 1101-139)

The title compound 1101-139 was prepared as a yellow solid (890 mg, 53% yield) from compound 1001 (1.7 g, 3.96 mmol) and methyl hex-5-ynoate (378 mg, 3.0 mmol) using a procedure similar to that described for compound 1101-138 (Example 57): LCMS: 428 [M+1]⁺.

### Step 58b. 6-(4-(3-Chloro-4-fluorophenylamino)-7-methoxyquinazolin-6-yl)-N-hydroxyhex-5-ynamide (Compound 139)

The title compound 139 was prepared as a white solid (80 mg, 73%) from compound 1101-139 (110 mg, 0.26 mmol) and freshly prepared hydroxylamine in methanol (3.0 mL) using a procedure similar to that described for compound 138 (Example 57): ¹H NMR (DMSO-d₆) δ 10.42 (s, 1H), 9.91 (s, 1H), 8.70 (s, 1H), 8.60 (s, 1H), 8.58 (s, 1H), 8.16-8.19 (m, 1H), 7.78-7.85 (m, 1H), 7.43 (t, J=9.0 Hz, 1H), 7.20 (s, 1H), 3.95 (s, 3H), 2.51 (t, J=7.2 Hz, 2H), 2.15 (t, J=7.2 Hz, 2H), 1.75-1.84 (m, 2H). LC-MS: 429 (M+1).

### Example 59

### Preparation of 5-(4-(3-chloro-4-fluorophenylamino)-7-methoxyquinazolin-6-yl)-N-hydroxypentanamide (compound 144)

### Step 59a. Methyl 6-(4-(3-chloro-4-fluorophenylamino)-7-methoxyquinazolin-6-yl)hex-5-ynoate (Compound 1102-144)

To a solution of 1101-138 (500 mg, 0.21 mmol) in methanol (30 mL) was added 50 mg of Pd/C (10%). The mixture was stirred at room temperature under hydrogen atmosphere (1 atm) for 16 h. The mixture was filtered, and the filtrate was concentrated to give the crude 1102-144 (480 mg, 94% yield) which was used directly in the next step. LC-MS: 418 (M+1).

### Step 59b. 5-(4-(3-Chloro-4-fluorophenylamino)-7-methoxyquinazolin-6-yl)-N-hydroxypentanamide (Compound 144)

To a flask containing compound 1102-144 (480 mg, 1.14 mmol) was added a solution of freshly prepared hydroxylamine in methanol (5.0 mL). The mixture was stirred at room temperature for 0.5 h. It was then convert to PH 7 using acetic acid. The resulting solid was filtered, washed with methanol to yield the product 144 as a white solid (400 mg, 83% yield). ¹H NMR (DMSO-d₆) δ 10.34 (s, 1H), 9.69 (s, 1H), 8.68 (s, 1H), 8.53 (s, 1H), 8.24 (s, 1H), 8.19-8.23 (m, 1H), 7.80-7.88 (m, 1H), 7.41 (t, J=9.0 Hz, 1H), 7.17 (s, 1H), 3.94 (s, 3H), 2.71 (t, J=6.6 Hz, 2H), 2.00 (t, J=7.2 Hz, 2H), 1.58-1.60 (m, 4H), 1.26-1.36 (m, 2H). LC-MS: 419 (M+1).

### Example 60

### Preparation of 5-(4-(3-chloro-4-fluorophenylamino)-7-methoxyquinazolin-6-yl)-N-hydroxypentanamide (Compound 145)

### Step 60a. Methyl 6-(4-(3-chloro-4-fluorophenylamino)-7-methoxyquinazolin-6-yl)hex-5-ynoate (Compound 1102-145)

The title compound 1102-145 was prepared as a crude product (210 mg, 99% yield) from compound 1101-139 (215 mg, 0.5 mmol) using a procedure similar to that described for compound 1102-144 (Example 59): LCMS: 432 [M+1]⁺.

### Step 60b. 5-(4-(3-Chloro-4-fluorophenylamino)-7-methoxyquinazolin-6-yl)-N-hydroxypentanamide (Compound 145)

The title compound 145 was prepared as a white solid (90 mg, 43%) from compound 1102-145 (210 mg, 0.5 mmol) and freshly prepared hydroxylamine in methanol (3.0 mL) using a procedure similar to that described for compound 144 (Example 59): ¹H NMR (DMSO-d₆) δ 10.33 (s, 1H), 9.68 (s, 1H), 8.66 (s, 1H), 8.52 (s, 1H), 8.21 (s, 1H), 8.15-8.19 (m, 1H), 7.80-7.85 (m, 1H), 7.41 (t, J=9.0 Hz, 1H), 7.16 (s, 1H), 3.93 (s, 3H), 2.71 (t, J=7.2 Hz, 2H), 1.95 (t, J=7.2 Hz, 2H), 1.50-1.67 (m, 4H), 1.26-1.36 (m, 2H). LC-MS: 433 (M+1).

### Example 61

### Preparation of 4-(4-(3-chloro-4-fluorophenylamino)-7-methoxyquinazolin-6-ylthio)-N-hydroxybutanamide (Compound 149)

### Step 61a. S-4-(3-Chloro-4-fluorophenylamino)-7-methoxyquinazolin-6-yl benzothioate (Compound 1201)

A mixture of compound 1001 (4.8 g, 11.4 mmol), thiobenzoic acid (7.8 g, 56.9 mol), 1,10-phenathroline (0.45 g, 2.3 mmol), copper iodide (0.22 g, 1.1 mmol) and DIPEA (2.94 g, 22.8 mmol) in toluene (20 mL) was stirred at 110° C. for 24 h under nitrogen atmosphere. After completion, the solvent was removed with reduced pressure and the residue was purified by column chromatography to get the crude target compound as a brown solid (1.0 g, 20%). LCMS: 440 [M+1]⁺.

### Step 61b. Ethyl 2-(4-(3-chloro-4-fluorophenylamino)-7-methoxyquinazolin-6-ylthio)acetate (Compound 1202-149)

A mixture of compd. 1201 (0.3 g, 0.68 mmol) and K₂CO₃ (0.14 g, 1.0 mmol) in DMF was stirred at 50° C. for 6 h under nitrogen. Ethyl 4-bromobutanoate (0.14 g, 0.71 mmol) was then added with a syringe and stirred for another 3 h. After the completion of the reaction, the solvent was removed with reduced pressure and the residue was purified by column chromatography to give the target compound 1202-149 as a pale yellow solid (50 mg, 16%). LCMS: 450 [M+1]⁺.

### Step 61c. 4-(4-(3-Chloro-4-fluorophenylamino)-7-methoxyquinazolin-6-ylthio)-N-hydroxybutanamide (Compound 149)

A mixture of compound 1202-149 (48 mg, 0.11 mmol) and freshly prepared NH₂OH methanol solution (1.77 M, 3.5 mL) was stirred for 30 min at room temperature. The mixture was adjusted to pH=7.0 with AcOH and the solvent was removed. The solid was collected and purified by column chromatography to give the target compound 149 as a pale yellow powder (14 mg, 30%). LCMS: 437.7 [M+1]⁺; ¹H NMR (DMSO-d₆) δ 10.72 (s, 1H), 9.82 (s, 1H), 8.94 (s, 1H), 8.55 (s, 1H), 8.38 (m, 1H), 8.19 (s, 1H), 8.06 (m, 1H), 7.39 (m, 1H), 7.20 (s, 1H), 3.97 (s, 3H), 3.03 (m, 2H), 2.22 (m, 2H), 1.91 (brs, 2H).

### Example 62

### Preparation of 5-(4-(3-chloro-4-fluorophenylamino)-7-methoxyquinazolin-6-ylthio)-N-hydroxypentanamide (Compound 151)

### Step 62a. Ethyl 5-(4-(3-chloro-4-fluorophenylamino)-7-methoxyquinazolin-6-ylthio)pentanoate (Compound 1202-151)

The title compound 1202-151 was prepared as a pale yellow solid (90 mg, 28% yield) from compound 1201 (300 mg, 0.68 mmol) using a procedure similar to that described for compound 1202-149 (Example 61): LCMS: 464 [M+1]⁺.

### Step 62b. 5-(4-(3-Chloro-4-fluorophenylamino)-7-methoxyquinazolin-6-ylthio)-N-hydroxypentanamide (Compound 151)

The title compound 151 was prepared as a pale yellow powder (25 mg, 29%) from compound 1202-151 (87 mg, 0.19 mmol) and freshly prepared hydroxylamine in methanol (1.77M, 4.0 mL) using a procedure similar to that described for compound 149 (Example 61): LCMS: 451.7 [M+1]⁺; ¹H NMR (DMSO-d₆) δ 10.74 (brs, 1H), 10.40 (s, 1H), 8.75 (s, 1H), 8.21 (s, 1H), 7.99 (m, 1H), 7.67 (m, 1H), 7.52 (m, 1H), 7.20 (s, 1H), 4.01 (s, 3H), 3.12 (brs, 2H), 2.00 (brs, 2H), 1.67 (brs, 4H).

### Example 63

### Preparation of 5-(4-(3-chloro-4-fluorophenylamino)-7-methoxyquinazolin-6-ylthio)-N-hydroxypentanamide (Compound 155)

### Step 63a. Ethyl 2-(4-(3-chloro-4-fluorophenylamino)-7-methoxyquinazolin-6-ylthio)acetate (Compound 1202-155)

The title compound 1202-155 was prepared as a pale yellow solid (87 mg, 26% yield) from compound 1201 (300 mg, 0.68 mmol) using a procedure similar to that described for compound 1202-149 (Example 61): LCMS: 492 [M+1]⁺.

### Step 63b. 7-(4-(3-Chloro-4-fluorophenylamino)-7-methoxyquinazolin-6-ylthio)-N-hydroxyheptanamide (Compound 155)

The title compound 155 was prepared as a pale yellow powder (28 mg, 34%) from compound 1202-155 (85 mg, 0.19 mmol) and freshly prepared hydroxylamine in methanol (1.77M, 4.0 mL) using a procedure similar to that described for compound 149 (Example 61): LCMS: 479.7 [M+1]⁺; ¹H NMR (DMSO-d⁶) δ 10.32 (brs, 1H), 9.76 (s, 1H), 8.65 (s, 1H), 8.51 (s, 1H), 8.14 (s, 1H), 8.09 (m, 1H), 7.75 (m, 1H), 7.44 (m, 1H), 7.19 (s, 1H), 3.97 (s, 3H), 3.08 (m, 2H), 1.92 (brs, 2H), 1.64 (brs, 2H), 1.45 (m, 4H), 1.28 (m, 2H).

### Example 64

### Preparation of 7-(4-(3-chloro-4-fluorophenylamino)-6-methoxyquinazolin-7-yloxy)-N-hydroxyheptanamide (Compound 161)

### Step 64a. Ethyl 4-(7-ethoxy-7-oxoheptyloxy)-3-hydroxybenzoate (Compound 1301-161)

To a solution of ethyl 3,4-dihydroxybenzoate 0401 (6.0 g, 33 mmol) in DMF (50 mL) was added potassium carbonate (4.6 g, 33 mmol). The mixture was stirred at room temperature for 15 min, and then a solution of ethyl 7-bromoheptanoate (7.821 g, 33 mmol) in DMF (10 mL) was added dropwise. The mixture was stirred for 12 hours at 20° C. After reaction the mixture was filtered, and the filtrate was concentrated in vacuo. The resulting residue was dissolved in dichloromethane and washed with brine. The organic phase was collected and dried over sodium sulfate, filtered and concentrated to give crude product. The crude product was purified by column chromatography (ethyl acetate/petroleum ether=1:10) to give the title product 1301-161 as a white solid (2.44 g, 22%): LCMS: 338 [M+1]⁺, ¹H NMR (DMSO-d₆): δ 1.177 (t, J=7.2 Hz, 3H), 1.247-1.438 (m, 7H), 1.480-1.553 (m, 2H), 1.579-1.754 (m, 2H), 2.245-2.294 (t, J=7.2 Hz, 2H), 3.972-4.063 (m, 4H), 4.190-4.261 (q, J=7.2, 14.1 Hz 2H), 6.958-6.986 (d, J=8.4 Hz, 1H), 7.358-7.404 (m, 2H), 9.36 (s, 1H).

### Step 64b. Ethyl 4-(7-ethoxy-7-oxoheptyloxy)-3-methoxybenzoate (Compound 1302-161)

Compound 1301-161 (1.2 g, 3.55 mmol), iodomethane (0.504 g, 3.55 mmol) and potassium carbonate (1.47 g, 10.65 mmol) in DMF (15 mL) was stirred at 80° C. for 3 hours. After reaction the mixture was filtrated. The filtrate was concentrated in vacuo, and the resulting residue was dissolved in dichloromethane and washed with brine twice. The organic phase was collected and dried over sodium sulfate, filtered and concentrated to give the title product 1302-161 as a white solid (1.2 g, 97%): LCMS: 353 [M+1]⁺, ¹H NMR (DMSO-d₆): δ1.131-1.178 (t, J=6.9 Hz, 3H), 1.267-1.395 (m, 7H), 1.478-1.574 (m, 2H), 1.665-1.755 (m, 2H), 2.242-2.291 (t, J=7.2 Hz, 2H), 3.792 (s, 3H), 3.982-4.063 (m, 4H), 4.229-4.300 (q, J=7.2 Hz, 2H), 7.025-7.052 (d, J=8.1 Hz, 1H), 7.418-7.424 (d, J=1.8 Hz, 1H), 7.529-7.562 (dd, J=8.4 Hz, 1.8 Hz, 1H).

### Step 64c. Ethyl 4-(7-ethoxy-7-oxoheptyloxy)-5-methoxy-2-nitrobenzoate (Compound 1303-161)

To a stirred solution of compound 1302-161 (1.2 g, 3.47 mmol) in acetic acid (10 mL) at 20° C. was added fuming nitric acid (2.18 g, 34.7 mmol) dropwise. The reaction mixture was stirred at 20° C. for 1 hour and was then poured into ice-water and extracted with dichloromethane twice. The combined organic phase was washed with brine, aqueous NaHCO₃ solution and brine, and dried over sodium sulfate, filtered and concentrated to give the title product 1303-161 as a yellow oil (1.375 g, 98%): LCMS: 398 [M+1]⁺.

### Step 64d. Ethyl 2-amino-4-(7-ethoxy-7-oxoheptyloxy)-5-methoxybenzoate (Compound 1304-161)

A mixture of 1303-161 (1.375 g, 3.46 mmol), ethanol (30 mL), water (10 mL) and hydrogen chloride (1 mL) was stirred to form a clear solution. To the above solution was added powder iron (2.0 g, 34.6 mmol) portionwise. The mixture was stirred at reflux for 30 min, and was then cooled to room temperature. The pH of the reaction mixture was adjusted to 8 with the addition of 10% sodium hydroxide solution and filtered. The filtrate was concentrated to remove ethanol and then extracted with dichloromethane twice. The combined organic phase was washed with brine and dried over sodium sulfate, filtered and concentrated to give the title product 1304-161 as a yellow solid (1.07 g, 84%): LCMS: 368 [M+1]⁺.

### Step 64e. Ethyl 7-(6-methoxy-4-oxo-3,4-dihydroquinazolin-7-yloxy)heptanoate (Compound 1305-161)

A mixture of compound 1304-161 (1.07 g, 2.92 mmol), ammonium formate (0.184 g, 3 mmol) and formamide (10 mL) was stirred at 180° C. for 3 hours. After reaction the mixture was cooled to room temperature. The formamide was removed under reduce pressure, and the residue was dissolved in dichloromethane and washed with brine. The organic phase was dried over sodium sulfate, filtered and concentrated to give the title product 1305-161 as a brown solid (0.684 g, 67%): LCMS: 349 [M+1]⁺.

### Step 64f. Ethyl 7-(4-chloro-6-methoxyquinazolin-7-yloxy)heptanoate (Compound 1306-161)

A mixture of product 1305-161 (0.684 g, 1.97 mmol) and phosphoryl trichloride (20 mL) was stirred at reflux for 4 hours. After reaction the excessive phosphoryl trichloride was removed under reduced pressure and the residue was dissolved in dichloromethane and washed with water, aqueous NaHCO₃ solution and brine. The organic phase was dried over sodium sulfate, filtered and concentrated to give the title product 1306-161 as a yellow solid (0.59 g, 82%): LCMS: 367 [M+1]⁺.

### Step 64g. Ethyl 7-(4-(3-chloro-4-fluorophenylamino)-6-methoxyquinazolin-7-yloxy)heptanoate (Compound 1307-161)

A mixture of 1306-161 (336 mg, 0.92 mmol) and 3-chloro-4-fluorobenzenamine (140 mg, 0.92 mmol) in isopropanol (10 mL) was stirred at reflux for 4 hours. After reaction the mixture was cooled to room temperature and resulting precipitate was isolated, washed with isopropanol and ether, and dried to give the title compound 1307-161 as a yellow solid (389 mg, 89%): LCMS: 476 [M+1]⁺.

### Step 64h. 7-(4-(3-chloro-4-fluorophenylamino)-6-methoxyquinazolin-7-yloxy)-N-hydroxyheptanamide (Compound 161)

To a freshly prepared hydroxylamine solution (2.5 mL, 3.75 mmol) was added compound 1307-161 (359 mg, 0.75 mmol). The resulting reaction mixture was stirred at 25° C. for 24 hours. After reaction the mixture was neutralized with acetic acid, and resulting precipitate was isolated, washed with water, and dried to give the title compound 161 as a white solid (60 mg, 17%): mp 238.5˜253.4° C., LCMS: 463 [M+1]⁺, ¹H NMR (DMSO-d₆): δ1.23-1.55 (m, 6H), 1.76-1.8 (m, 2H), 1.96 (t, J=7.2 Hz, 2H), 3.96 (s, 3H), 4.13 (t, J=6.3 Hz, 2H), 7.19 (s, 1H), 7.20 (m, 2H), 7.46 (t, J=9 Hz, 1H), 7.78 (d, J=7.5 Hz, 1H), 8.12-8.15 (dd, J=2.4, 6.9 Hz, 1H), 8.50 (s, 1H), 8.67 (s, 1H), 9.57 (s, 1H), 10.35 (s, 1H).

### Example 65

### Preparation of 7-(4-(3-ethynylphenylamino)-6-methoxyquinazolin-7-yloxy)-N-hydroxyheptanamide (Compound 162)

### Step 65a. Ethyl 7-(4-(3-ethynylphenylamino)-6-methoxyquinazolin-7-yloxy)heptanoate (Compound 1307-162)

The title compound 1307-162 was prepared as a yellow solid (253 mg, 46% yield) from compound 1306-162 (446 mg, 1.22 mmol), 3-ethynylbenzenamine (142 mg, 1.22 mmol) and i-propanol (10 mL) using a procedure similar to that described for compound 1307-161 (Example 64): LCMS: 448 [M+1]⁺.

### Step 65b. 7-(4-(3-ethynylphenylamino)-6-methoxyquinazolin-7-yloxy)-N-hydroxyheptanamide (Compound 162)

The title compound 162 was prepared as a yellow powder (20 mg, 8%) from compound 1307-161 (246 mg, 0.0.55 mmol) and freshly prepared hydroxylamine in methanol (2.0 mg, 2.75 mmol) using a procedure similar to that described for compound 161 (Example 64): LCMS: 435 [M+1]⁺, ¹H NMR (DMSO-d₆): δ1.301-1.541 (m, 6H), 1.740-1.792 (m, 2H), 1.929-1.977 (m, 2H), 3.959 (s, 3H), 4.123 (t, J=6.6 Hz, 2H), 4.192 (s, 1H), 7.176-7.221 (m, 2H), 7.360-7.427 (m, 1H), 7.831-7.890 (m, 2H), 7.966 (m, 1H), 8.504 (s, 1H), 8.642 (s, 1H), 9.547 (s, 1H), 10.321 (s, 1H).

### Example 66

### Preparation of 7-(4-(3-chloro-4-fluorophenylamino)-6-(2-methoxyethoxy)quinazolin-7-yloxy)-N-hydroxyheptanamide (Compound 167)

### Step 66a. Ethyl 4-(7-ethoxy-7-oxoheptyloxy)-3-(2-methoxyethoxy)benzoate (Compound 1302-167)

The title compound 1302-167 was prepared as a yellow solid (1400 mg, 97% yield) from compound 1301 (1223 mg, 3.62 mmol), 2-methoxyethyl 4-methylbenzenesulfonate (0.834, 3.62 mmol), DMF (15 mL) and potassium carbonate (1.50 g, 10.86 mmol) using a procedure similar to that described for compound 1302-161 (Example 64): LCMS: 397 [M+1]⁺, ¹H NMR (DMSO-d₆): δ1.152 (t, J=7.2 Hz, 3H), 1.264-1.405 (m, 7H), 1.478-1.572 (m, 2H), 1.663-1.730 (m, 2H), 2.267 (t, J=7.2 Hz, 2H), 3.315 (s, 3H), 3.650 (t, J=5.4 Hz, 2H), 3.990-4.062 (m, 4H), 4.089-4.119 (m, 3H), 4.222-4.293 (q, J=7.2 Hz, 2H), 7.053 (d, J=8.1 Hz, 1H), 7.447-7.486 (m, 1H), 7.539-7.567 (dd, J=8.4 Hz, 1.8 Hz, 1H).

### Step 66b. Ethyl 4-(7-ethoxy-7-oxoheptyloxy)-5-(2-methoxyethoxy)-2-nitrobenzoate (Compound 1303-167)

The title compound 1303-167 was prepared as a yellow oil (1510 mg, 97% yield) from compound 1302-167 (1400 mg, 3.5 mmol), acetic acid (10 mL) and fuming nitric acid using a procedure similar to that described for compound 1303-161 (Example 64): LCMS: 442 [M+1]⁺.

### Step 66c. Ethyl 2-amino-4-(7-ethoxy-7-oxoheptyloxy)-5-(2-methoxyethoxy)benzoate (Compound 1304-167)

The title compound 1304-167 was prepared as a yellow oil (1210 mg, 97% yield) from compound 1303-167 (1500 mg, 3.4 mmol), powder iron (1.9 g, 34 mmol), ethanol (30 mL), water (10 mL) and hydrogen chloride (1 mL) using a procedure similar to that described for compound 1304-161 (Example 64): LCMS: 412 [M+1]⁺.

### Step 66d. Ethyl 7-(6-(2-methoxyethoxy)-4-oxo-3,4-dihydroquinazolin-7-yloxy)heptanoate (Compound 1305-167)

The title compound 1305-167 was prepared as a yellow solid (859 mg, 85% yield) from compound 1304-167 (1210 mg, 2.9 mmol), ammonium formate (0.184 g, 3 mmol) and formamide (10 mL) using a procedure similar to that described for compound 1305-161 (Example 64): LCMS: 393 [M+1]⁺.

### Step 66e. Ethyl 7-(4-chloro-6-(2-methoxyethoxy)quinazolin-7-yloxy)heptanoate (Compound 1306-167)

The title compound 1306-167 was prepared as a yellow solid (572 mg, 63% yield) from compound 1305-167 (859 mg, 2.2 mmol) and phosphoryl trichloride (20 mL) using a procedure similar to that described for compound 1306-161 (Example 64): LCMS: 411 [M+1]⁺.

### Step 66f. Ethyl 7-(4-(3-chloro-4-fluorophenylamino)-6-(2-methoxyethoxy)quinazolin-7-yloxy)heptanoate (Compound 1307-167)

The title compound 1307-167 was prepared as a yellow solid (238 mg, 76% yield) from compound 1306-167 (251 mg, 0.6 mmol), 3-chloro-4-fluorobenzenamine (90 mg, 0.6 mmol) and i-propanol (5 mL) using a procedure similar to that described for compound 1307-161 (Example 64): LCMS: 520 [M+1]⁺.

### Step 66g. 7-(4-(3-chloro-4-fluorophenylamino)-6-(2-methoxyethoxy)quinazolin-7-yloxy)-N-hydroxyheptanamide (Compound 167)

The title compound 167 was prepared as a yellow solid (20 mg, 9% yield) from compound 1307-167 (232 mg, 0.45 mmol) and) and freshly prepared hydroxylamine solution (2 mL, 2.1 mmol) using a procedure similar to that described for compound 161 (Example 64): LCMS: 507 [M+1]⁺, ¹H NMR (DMSO-d₆): δ 1.314-1.539 (m, 6H), 1.754-1.801 (m, 2H), 1.926-1.975 (m, 2H), 3.368 (s, 3H), 3.770 (t, J=4.8 Hz, 2H), 4.135 (t, J=6.3 Hz, 2H), 4.267 (t, J=4.8 Hz, 2H), 7.19 (s, 1H), 7.440 (t, J=8.4 Hz, 1H), 7.764-7.833 (m, 2H), 8.095-8.126 (dd, J=2.7, 6.9 Hz, 1H), 8.499 (s, 1H), 8.612 (s, 1H), 8.635 (s, 1H), 9.555 (s, 1H), 10.314 (s, 1H).

### Example 67

### Preparation of 7-(4-(3-ethynylphenylamino)-6-(2-methoxyethoxy)quinazolin-7-yloxy)-N-hydroxyheptanamide (Compound 168)

### Step 67a. Ethyl 7-(4-(3-ethynylphenylamino)-6-(2-methoxyethoxy)quinazolin-7-yloxy)Heptanoate (Compound 1307-168)

The title compound 1307-168 was prepared as a yellow solid (214 mg, 56% yield) from compound 1307-167 (320 mg, 0.78 mmol), 3-ethynylbenzenamine (92 mg, 0.78 mmol), i-propanol (5 mL): using a procedure similar to that described for compound 1307-161 (Example 64): LCMS: 520 [M+1].

### Step 67b. 7-(4-(3-ethynylphenylamino)-6-(2-methoxyethoxy)quinazolin-7-yloxy)-N-hydroxyheptanamide (Compound 168)

The title compound 168 was prepared as a yellow solid (30 mg, 15% yield) from compound 1307-178 (204 mg, 0.42 mmol) and) and freshly prepared hydroxylamine solution (2 mL, 2.1 mmol) using a procedure similar to that described for compound 161 (Example 64): LCMS: 479 [M+1]⁺, ¹H NMR (DMSO-d₆): δ1.314-1.539 (m, 6H), 1.754-1.800 (m, 2H), 1.925-1.975 (m, 2H), 3.370 (s, 3H), 3.771 (t, J=4.8 Hz, 2H), 4.131 (t, J=6.3 Hz, 2H), 4.186 (s, 1H), 4.275 (t, J=4.8 Hz, 2H), 7.19 (d, J=7.5 Hz, 2H), 7.390 (t, J=7.8 Hz, 1H), 7.847-7.900 (m, 2H), 7.975 (s, 1H), 8.487 (s, 1H), 8.636 (s, 1H), 9.455 (s, 1H), 10.316 (s, 1H).

### Example 68

### Preparation of 3-((5-(4-(3-chloro-4-(3-fluorobenzyloxy)phenylamino)quinazolin-6-yl)furan-2-yl)methylamino)-N-hydroxypropanamide (Compound 174)

### Step 68a. Methyl 2-amino-5-iodobenzoate (Compound 1402-174

Methyl 2-aminobenzoate (23 g, 15.2 mmol) was dissolved in 200 mL of water and 32 mL of concentrated hydrochloric acid; the solution was cooled to 20° C. A solution of iodine monochloride in hydrochloric acid is prepared by diluting 28 mL of concentrated hydrochloric acid with 100 mL of cold water, adding just sufficient crushed ice to bring the temperature to 5° C., and, during about two minutes, stirring in monochloride (25 g, 15.5 mmol). The iodine monochloride solution is stirred rapidly into the methyl 2-aminobenzoate solution. Methyl 2-amino-5-iodobenzoate separates almost immediately as a granular, tan to violet precipitate. The mixture is stirred for an hour, then filtered, washed with cold water, and then dried in vacuum to yield the 1402-174 as a solid (17.8 g, 42%): LC-MS: 278 [M+1]⁺, ¹H NMR (DMSO-d₆): δ 3.70 (s, 3H), 6.64 (d, J=9.0 Hz, 1H), 6.78 (b, 2H), 7.47 (dd, J₁=9.0 Hz, J₂=1.8 Hz, 1H), 7.90 (d, J=1.8 Hz, 1H).

### Step 68b. 6-Iodoquinazolin-4(3H)-one (Compound 1403-174)

Methyl 2-amino-5-iodobenzoate (17.8 g, 64 mmol) was heated in 300 mL of formamide at 190° C. for 2 hours. The mixture was cooled to room temperature and the solid product was filtrated and dried in vacuum. The formed product 1403-174 was used without further purification. (10 g, 56.1%): LC-MS: 273 [M+1]⁺, ¹H NMR (DMSO-d₆): δ 7.46 (d, J=9.0 Hz, 1H), 8.10 (m, 2H), 8.36 (d, J=2.1 Hz, 1H), 12.40 (s, 1H).

### Step 68c. 4-Chloro-6-iodoquinazoline (Compound 1404-174)

6-Iodoquinazolin-4(3H)-one (10 g, 37 mmol) was refluxed in POCl₃ (100 mL) overnight. Then POCl₃ was removed in vacuo. The residue was dissolved in CH₂Cl₂ (500 mL). The organic phase was washed with water (100 mL) and dried (MgSO₄). Then CH₂Cl₂ was removed in vacuo and 1404-174 was obtained (5.7 g, 53%): LC-MS: 291 [M+1]⁺, ¹H NMR (CDCl₃): δ 7.81 (d, J=9.0 Hz, 1H), 8.21 (dd, J₁=9.0 Hz, J₂=1.8 Hz, 1H), 8.65 (d, J=1.8 Hz, 1H), 9.06 (s, 1H).

### Step 68d. Synthesis of N-(3-chloro-4-(3-fluorobenzyloxy)phenyl)-6-iodoquinazolin-4-amine (Compound 1405-174)

4-Chloro-6-iodoquinazoline (5.7 g, 19.7 mmol) and 3-chloro-4-(3-fluorobenzyloxy)aniline (4.9 g, 19.7 mmol) was refluxed in isopropanol (150 mL) overnight. The mixture was cooled to room temperature. The solid product was precipitated, filtrated and dried in vacuum. The product 1405-174 was pure enough and used without further purification. (7.4 g, 74.2%): LC-MS: 506 [M+1]⁺, ¹H NMR (DMSO-d₆): δ 5.29 (s, 2H), 7.18 (m, 1H), 7.33 (m, 3H), 7.48 (m, 1H), 7.66 (m, 1H), 7.74 (d, J=9.0 Hz, 1H), 7.90 (d, J=2.2 Hz, 1H), 8.37 (d, J=9.0 Hz, 1H), 8.94 (s, 1H), 9.29 (s, 1H).

### Step 68e. 5-(4-(3-Chloro-4-(3-fluorobenzyloxy)phenylamino)quinazolin-6-yl)furan-2-carbaldehyde (Compound 1406-174)

N-(3-Chloro-4-(3-fluorobenzyloxy)phenyl)-6-iodoquinazolin-4-amine (387 mg, 0.77 mmol) and 5-formylfuran-2-ylboronic acid (129 mg, 0.92 mmol) were added into the mixture of THF (10 mL), ethanol (5 mL) and Et₃N (0.3 mL) under N₂ atmosphere. Then PdCl₂(dppf) (26 mg, 0.03 mmol) was added into the mixture. The mixture was refluxed overnight. Then the solvent was removed in vacuo, the residue was chromatographed on silica gel with ethyl acetate to give product 1406-174 (240 mg, 66.2%): LC-MS: 474 [M+1]⁺, ¹H NMR (DMSO-d₆): δ 5.20 (s, 2H), 7.17 (m, 1H), 7.29 (m, 3H), 7.41 (m, 2H), 7.74 (m, 2H), 7.86 (d, J=9.0 Hz, 1H), 7.97 (s, 1H), 8.31 (d, J=9.0 Hz, 1H), 8.56 (s, 1H), 8.96 (s, 1H), 9.66 (s, 1H), 10.11 (s, 1H).

### Step 68f. Ethyl 3-((5-(4-(3-chloro-4-(3-fluorobenzyloxy)phenylamino)quinazolin-6-yl)furan-2-yl)methylamino)propanoate (Compound 1407-174)

Compound 1406-174 (240 mg, 0.5 mmol) and ethyl 3-aminopropanoate hydrochloride (77 mg, 0.5 mmol) were dissolved in 10 mL of THF, then Et₃N (0.1 mL) was added. The mixture was stirred for 10 min. and then NaBH(AcO)₃ (148 mg, 0.7 mmol) was added into the mixture. The mixture was stirred for another 1 hour. The solvent was removed, and the residue was purified by chromatography on silica gel with CH₂Cl₂/MeOH (100:5) to give product 1407-174 (140 mg, 47.9%): LC-MS: 575 [M+1]⁻, ¹H NMR (DMSO-d₆): δ 1.13 (t, J=6.9 Hz, 3H), 2.43 (m, 2H), 2.80 (t, J=6.9 Hz, 3H), 3.76 (s, 2H), 4.01 (q, J=6.9 Hz, 2H), 5.21 (s, 2H), 6.46 (s, 1H), 7.03 (m, 1 H), 7.16 (m, 1H), 7.30 (m, 3H), 7.46 (m, 1H), 7.82 (m, 2H), 8.03 (m, 1H), 8.14 (m, 1H), 8.52 (s, 1H), 8.71 (s, 1H), 9.90 (s, 1H).

### Step 68g. 3-((5-(4-(3-Chloro-4-(3-fluorobenzyloxy)phenylamino)quinazolin-6-yl)furan-2-yl)methylamino)-N-hydroxypropanamide (Compound 174)

Compound 1407-174 (110 mg, 0.19 mmol) was dissolved in freshly made NH₂OH methanol solution (1 mL, 1.76 mol/L). The mixture was stirred for 30 min. and the reaction was monitored by TLC. HOAc was added to adjust the pH of the reaction mixture to 7. The solvent was removed in vacuo and the residue was washed with water (10 mL). The product was purified by preparative liquid chromatography to yield compound 174 as a yellow solid (41 mg, 37.2%): Mp. 170° C. LC-MS: 562 [M+1]⁺, ¹H NMR (DMSO-d₆): δ 2.14 (t, J=6.9 Hz, 2H), 2.77 (t, J=6.9 Hz, 2H), 3.79 (s, 2H), 5.25 (s, 2H), 6.45 (d, J=3.0 Hz, 1H), 7.03 (d, J=3.0 Hz, 1H), 7.18 (m, 1H), 7.31 (m, 3H), 7.45 (m, 1H), 7.72 (m, 2H), 8.00 (m, 1H), 8.15 (d, J=7.5 Hz, 1H), 8.53 (s, 1H), 8.71 (s, 2H), 9.92 (s, 1H).

### Example 69

### Preparation of 6-((5-(4-(3-chloro-4-(3-fluorobenzyloxy)phenylamino)quinazolin-6-yl)furan-2-yl)methylamino)-N-hydroxyhexanamide (Compound 177)

### Step 69a. Methyl 6-((5-(4-(3-chloro-4-(3-fluorobenzyloxy)phenylamino)quinazolin-6-yl)furan-2-yl)methylamino)hexanoate (Compound 1407-177)

The title compound 1407-177 was prepared (260 mg, 21.6% yield) from compound 1406-174 (960 mg, 2.0 mmol) and methyl 6-aminohexanoate hydrochloride (362 mg, 2 mmol) using a procedure similar to that described for compound 1407-174 (Example 68): LCMS: 603 [M+1]⁺.

### Step 69b. 6-((5-(4-(3-Chloro-4-(3-fluorobenzyloxy)phenylamino)quinazolin-6-yl)furan-2-yl)methylamino)-N-hydroxyhexanamide (compound 177)

The title compound 177 was prepared as a white solid (22 mg, 22% yield) from compound 1407-177 (100 mg, 0.17 mmol) and freshly prepared hydroxylamine solution (1 mL, 1.76 mol/L) using a procedure similar to that described for compound 174 (Example 68): Mp. 121° C. LC-MS: 604 [M+1]⁺, ¹H NMR (DMSO-d₆): δ 1.03 (t, J=6.0 Hz, 2H), 1.18 (m, 2H), 1.47 (m, 4H), 1.92 (t, J=6.0 Hz, 2H), 2.54 (m, 2H), 3.41 (s, 1H), 3.78 (s, 2H), 5.26 (s, 2H), 6.40 (s, 1H), 7.02 (s, 1H), 7.17 (m, 1H), 7.29 (m, 3H), 7.46 (m, 1H), 7.76 (m, 2H), 7.99 (s, 1H), 8.16 (d, J=8.1 Hz, 1H), 8.53 (s, 1H), 8.70 (m, 2H), 9.90 (s, 1H), 10.33 (s, 1H).

### Example 70

### Preparation of 7-((5-(4-(3-chloro-4-(3-fluorobenzyloxy)phenylamino)quinazolin-6-yl)furan-2-yl)methylamino)-N-hydroxyheptanamide (compound 178)

### Step 70a. Ethyl 7-((5-(4-(3-chloro-4-(3-fluorobenzyloxy)phenylamino)quinazolin-6-yl)furan-2-yl)methylamino)heptanoate (Compound 1407-178)

The title compound 1407-178 was prepared (270 mg, 21.4% yield) from compound 1406-174 (960 mg, 2.0 mmol) and methyl ethyl 7-aminoheptanoate hydrochloride hydrochloride (418 mg, 2 mmol) using a procedure similar to that described for compound 1407-174 (Example 68): LCMS: 631 [M+1]⁺.

### Step 70b. 7-((5-(4-(3-Chloro-4-(3-fluorobenzyloxy)phenylamino)quinazolin-6-yl)furan-2-yl)methylamino)-N-hydroxyheptanamide (Compound 178)

The title compound 178 was prepared as a white solid (25 mg, 25% yield) from compound 1407-178 (110 mg, 0.17 mmol) and freshly prepared hydroxylamine solution (1 mL, 1.76 mol/L) using a procedure similar to that described for compound 174 (Example 68): Mp. 120° C. LC-MS: 618 [M+1]⁺, ¹H NMR (DMSO-d₆): δ 1.22 (m, 4H), 1.42 (m, 4H), 1.90 (t, J=7.5 Hz, 2H), 2.54 (m, 2H), 3.76 (s, 2H), 5.24 (s, 2H), 6.42 (d, J=3.0 Hz, 1H), 7.01 (d, J=3.0 Hz, 1H), 7.19 (m, 1H), 7.31 (m, 3H), 7.44 (m, 1H), 7.70 (m, 2H), 7.99 (s, 1H), 8.14 (m, 1H), 8.52 (s, 1H), 8.69 (m, 2H), 9.89 (s, 1H), 10.30 (s, 1H).

### Example 71

### Preparation of 7-(4-(3-chloro-4-(3-fluorobenzyloxy)phenylamino)quinazolin-6-yloxy)-N-hydroxyheptanamide (Compound 198)

### Step 71a. 2-Chloro-1-(3-fluorobenzyloxy)-4-nitrobenzene (Compound 1502)

A mixture of 2-chloro-4-nitrophenol (35 g, 0.2 mol), m-furobenzylbromide (45.4 g, 0.24 mol), K₂CO₃ (55.2 g, 0.4 mol) and acetone (800 mL) was stirred at 30° C. for 16 h. The resulting mixture was filtered and washed with acetone. The filtrate was concentrated to give the crude product which was washed with petroleum ether and dried to give the product 1502 as a yellow solid (55.0 g, 99% yield). ¹H NMR (DMSO-d₆): δ 8.33 (d, J=3.3 Hz, 1H), 8.21-8.26 (m, 1H), 7.42-7.50 (m, 2H), 7.29-7.33 (m, 2H), 7.16-7.22 (m, 1H), 5.39 (s, 2H). LC-MS: 282 (M+1).

### Step 71b. 3-Chloro-4-(3-fluorobenzyloxy)benzenamine (Compound 1503)

A mixture of 1502 (15 g, 53.4 mmol), iron powder (30 g, 0.534 mol), concentrated hydrochloric acid (5.4 mL), ethanol (360 mL) and water (120 mL) was refluxed for 2 h. The hot solution was then filtered and the filtrate was concentrated to give the product 1503 as a solid (11.0 g, 82% yield). ¹H NMR (DMSO-d₆): δ 7.37-7.45 (m, 1H), 7.21-7.26 (m, 2H), 7.09-7.16 (m, 1H), 6.90 (d, J=8.7 Hz, 1H), 6.63-6.34 (m, 1H), 6.44 (dd, J₁, J₂=8.7 Hz, 1.8 Hz, 1H), 5.01 (s, 2H), 4.94 (s, 2H). LC-MS: 252 (M+1).

### Step 71c. Acetic acid 4-[3-chloro-4-(3-fluoro-benzyloxy)-phenylamino]-quinazolin-6-yl ester (Compound 1504-198)

A mixture of compound 0204 (Scheme 2) (0.85 g, 3.8 mmol) and 3-chloro-4-3-fluorobenzyloxy)benzenamine (1503) (1.26 g, 5.0 mmol) in isopropanol (20 mL) was stirred and heated at 90° C. for 20 minutes. The reaction was cooled to room temperature and the precipitate was isolated. The solid was washed with isopropanol and methanol, dried to provide the title compound 1504-198 as a dark yellow solid (1.5 g, 90%). LC-MS: 438 [M+1]⁺.

### Step 71d. 4-[3-Chloro-4-(3-fluoro-benzyloxy)-phenylamino]-quinazolin-6-ol (Compound 1505-198)

A mixture of compound 1504-198 (1.5 g, 3.4 mmol) and lithium hydroxide onohydrate (0.29 g, 6.9 mmol) in methanol (40 mL)/water (40 mL) was stirred at room temperature for 4 hours. The pH was adjusted to 4 with acetic acid and filtered. The collected yellow solid was washed by water and dried to obtained title compound 1505-198 as a yellow solid (1.2 g, 89%). LC-MS: 395 [M+1]⁺.

### Step 71e. 7-{4-[3-Chloro-4-(3-fluoro-benzyloxy)-phenylamino]-quinazolin-6-yloxy}-heptanoic acid ethyl ester (Compound 1506-198)

A mixture of compound 1505-198 (0.12 g, 0.30 mmol), ethyl 3-bromopropanoate (72 mg, 0.30 mmol) and K₂CO₃ (165 mg, 1.2 mmol) in DMF (5 mL) was stirred and heated to 60° C. overnight. The reaction was filtered and the filtrate was evaporated. The resulting solid was washed with ether and purified by TLC to obtain the title compound 1506-198 as a yellow solid (80 mg, 48%). LC-MS: 551 [M+1]⁺: ¹H NMR (DMSO-d₆): δ 1.15 (t, J=7.5 Hz, 3H), 1.46 (m, 8H), 1.79 (m, 2H), 2.29 (t, J=7.2 Hz, 2H), 3.24 (s, 1H), 4.02 (d, J₁=6.6 Hz, J₂=14.4 Hz, 2H), 4.12 (t, J=6.3 Hz, 2H), 5.24 (s, 2H), 7.15 (m, 1H), 7.45 (m, 3H), 7.48 (m, 2H), 7.85 (d, J=2.7 Hz, 1H), 7.98 (d, J=2.7 Hz, 1H), 8.47 (s, 1H), 9.57 (s, 1H).

### Step 71f. 7-{4-[3-Chloro-4-(3-fluoro-benzyloxy)-phenylamino]-quinazolin-6-yloxy}-heptanoic acid hydroxyamide (Compound 198)

To compound 1506-198 (70 mg, 0.13 mmol) was added the freshly prepared hydroxylamine methanol solution (0.5 mL, 0.89 mmol). The reaction process was monitored by TLC. After completion of the reaction, the mixture was neutralized with acetic acid and concentrated under reduce pressure to a residue which was washed by water to give the title compound 198 as a yellow solid (35 mg, 46%): LC-MS: 539 [M+1]⁻; ¹H NMR (DMSO-d₆): δ 1.50 (m, 8H), 1.79 (t, J=6.6 Hz, 2H), 3.24 (s, 1H), 1.95 (m, 2H), 4.12 (t, J=5.1 Hz, 2H), 5.24 (s, 2H), 7.15 (m, 1H), 7.45 (m, 3H), 7.48 (m, 2H), 7.70 (d, J=2.7 Hz, 1H), 7.87 (s, 1H), 7.97 (s, 1H), 8.50 (s, 1H), 8.67 (s, 1H), 9.70 (s, 1H), 10.35 (s, 1H).

### Example 72

### Preparation of 7-(4-(3-chloro-4-(3-fluorobenzyloxy)phenylamino)-7-methoxyquinazolin-6-yloxy)-N-hydroxyheptanamide (Compound 199)

### Step 72a. 4-(3-Chloro-4-(3-fluorobenzyloxy)phenylamino)-7-methoxyquinazolin-6-yl acetate (Compound 1504-199)

A mixture of 0105 (Scheme 1) (253 mg, 1.0 mmol) and 1503 (252 mg, 1.0 mmol) in isopropanol (10 mL) was stirred and heated to reflux for 1 hours. The mixture was cooled to room temperature and resulting precipitate was isolated. The solid was dried to give the title compound 1504-199 as a pale solid (420 mg, 90%): LCMS: 468 [m +1]⁻.

### Step 72b. 4-(3-Chloro-4-(3-fluorobenzyloxy)phenylamino)-7-methoxyquinazolin-6-ol (Compound 1505-199)

A mixture of compound 1504-199 (418 mg, 0.89 mmol), LiOH.H₂O (126 mg, 3.0 mmol) in methanol (20 mL) and H₂O (10 mL) was stirred at room temperature for 10 min. The mixture was neutralized by addition of dilution acetic acid. The precipitate was isolated and dried to give the title compound 1505-199 as a pale white solid (376 mg, 99%): LCMS: 426 [M+1]⁺; ¹H NMR (DMSO-d₆): δ 3.97 (s, 3H), 5.24 (s, 2H), 7.19 (m, 3H), 7.32 (m, 2H), 7.48 (m, 1H), 7.74 (m, 2H), 8.04 (d, J=2.4 Hz, 1H), 8.43 (s, 1H), 9.35 (s, 1H), 9.66 (s, 1H).

### Step 72c. Ethyl 7-(4-(3-chloro-4-(3-fluorobenzyloxy)phenylamino)-7-methoxyquinazolin-6-yloxy)heptanoate (Compound 1506-199)

A mixture of compound 1505-199 (170 mg, 0.4 mmol), ethyl 7-bromoheptanoate (95 mg, 0.4 mmol) and potassium carbonate (166 mg, 1.2 mmol) in N,N-dimethylformamide (10 mL) was stirred and heated to 70° C. for 4 hours. The reaction mixture was filtrated. The filtrate was concentrated under reduce pressure. The residues was suspended in water, the precipitate was collected and dried to give the title compound 1506-199 as a yellow solid (89 mg, 38%): LCMS: 582 [M+1]⁺.

### Step 72d. 7-(4-(3-Chloro-4-(3-fluorobenzyloxy)phenylamino)-7-methoxyquinazolin-6-yloxy)-N-hydroxyheptanamide (Compound 199)

A mixture of compound 1506-199 (88 mg, 0.15 mmol) and freshly prepared 1.77 mol/L NH₂OH/MeOH (3 mL, 5.3 mmol) was stirred at room temperature for 0.5 h. The reaction mixture was neutralized with AcOH, the precipitate was isolated and dried to give the title compound 199 as a pale yellow solid (48 mg, 56%): LCMS: 569 [M+1], ¹H NMR (DMSO-d₆): δ 1.35 (m, 2H), 1.50 (m, 4H), 1.83 (m, 2H), 1.98 (m, 2H), 3.94 (s, 3H), 4.13 (m, 2H), 5.26 (s, 2H), 7.19 (m, 2H), 7.36 (m, 3H), 7.48 (m, 1H), 7.69 (m, 1H), 7.80 (s, 1H), 7.95 (d, J=2.7 Hz, 1H), 8.45 (s, 1H), 8.68 (s, 1H), 9.43 (s, 1H), 10.36 (s, 1H).

### Biological Assays:

As stated hereinbefore the derivatives defined in the present invention possess anti-proliferation activity. These properties may be assessed, for example, using one or more of the procedures set out below:

(a) An In Vitro Assay which Determines the Ability of a Test Compound to Inhibit EGFR Kinase.

The ability of compounds to inhibit receptor kinase (EGFR) activity was assayed using HTScan™ EGF Receptor Kinase Assay Kits (Cell Signaling Technologies, Danvers, Mass.). EGFR tyrosine kinase was obtained as GST-kinase fusion protein which was produced using a baculovirus expression system with a construct expressing human EGFR(His672-Ala1210) (GenBank Accession No. NM_{—}005228) with an amino-terminal GST tag. The protein was purified by one-step affinity chromatography using glutathione-agarose. An anti-phosphotyrosine monoclonal antibody, P-Tyr-100, was used to detect phosphorylation of biotinylated substrate peptides (EGFR, Biotin-PTP1B (Tyr66). Enzymatic activity was tested in 60 mM HEPES, 5 mM MgCl₂ 5 mM MnCl₂ 200 μM ATP, 1.25 mM DTT, 3 μM Na₃VO₄, 1.5 mM peptide, and 50 ng EGF Recpetor Kinase. Bound antibody was detected using the DELFIA system (PerkinElmer, Wellesley, Mass.) consisting of DELFIA® Europium-labeled Anti-mouse IgG (PerkinElmer, #AD0124), DELFIA® Enhancement Solution (PerkinElmer, #1244-105), and a DELFIA® Streptavidin coated, 96-well Plate (PerkinElmer, AAAND-0005). Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm.

Test compounds were dissolved in dimethylsulphoxide (DMSO) to give a 20 mM working stock concentration. Each assay was setup as follows: Added 100 μl of 10 mM ATP to 1.25 ml 6 mM substrate peptide. Diluted the mixture with dH₂0 to 2.5 ml to make 2×ATP/substrate cocktail ([ATP]=400 mM, [substrate]=3 mM). Immediately transfer enzyme from −80° C. to ice. Allowed enzyme to thaw on ice. Microcentrifuged briefly at 4° C. to bring liquid to the bottom of the vial. Returned immediately to ice. Added 10 μl of DTT (1.25 mM) to 2.5 ml of 4×HTScan™ Tyrosine Kinase Buffer (240 mM HEPES pH 7.5, 20 mM MgCl₂, 20 mM MnCl, 12 mM NaVO₃) to make DTT/Kinase buffer. Transfer 1.25 ml of DTT/Kinase buffer to enzyme tube to make 4× reaction cocktail ([enzyme]=4 ng/μL in 4× reaction cocktail). Incubated 12.5 μl of the 4× reaction cocktail with 12.5 μl/well of prediluted compound of interest (usually around 10 μM) for 5 minutes at room temperature. Added 25 μl of 2×ATP/substrate cocktail to 25 μl/well preincubated reaction cocktail/compound. Incubated reaction plate at room temperature for 30 minutes. Added 50 μl/well Stop Buffer (50 mM EDTA, pH 8) to stop the reaction. Transferred 25 μl of each reaction and 75 μl dH₂O/well to a 96-well streptavidin-coated plate and incubated at room temperature for 60 minutes. Washed three times with 200 μl/well PBS/T (PBS, 0.05% Tween-20). Diluted primary antibody, Phospho-Tyrosine mAb (P-Tyr-100), 1:1000 in PBS/T with 1% bovine serum albumin (BSA). Added 100 μl/well primary antibody. Incubated at room temperature for 60 minutes. Washed three times with 200 μl/well PBS/T. Diluted Europium labeled anti-mouse IgG 1:500 in PBS/T with 1% BSA. Added 100 μl/well diluted antibody. Incubated at room temperature for 30 minutes. Washed five times with 200 μl/well PBS/T. Added 100 μl/well DELFIA® Enhancement Solution. Incubated at room temperature for 5 minutes. Detected 615 nm fluorescence emission with appropriate Time-Resolved Plate Reader.

(b) An In Vitro Assay which Determines the Ability of a Test Compound to Inhibit the EGF-Stimulated EGFR Phosphorylation.

Allowed A431 cell growth in a T75 flask using standard tissue culture procedures until cells reach near confluency (˜1.5×10⁷) cells; D-MEM, 10% FBS). Under sterile conditions dispensed 100 μl of the cell suspension per well in 96-well microplates (x cells plated per well). Incubated cells and monitor cell density until confluency is achieved with well-to-well consistency; approximately three days. Removed complete media from plate wells by aspiration or manual displacement. Replaced media with 50 μl of pre-warmed serum free media per well and incubated 4 to 16 hours. Made two fold serial dilutions of inhibitor using pre-warmed D-MEM so that the final concentration of inhibitor range from 10 μM to 90 pM. Removed media from A431 cell plate. Added 100 μl of serial diluted inhibitor into cells and incubate 1 to 2 hours. Removed inhibitor from plate wells by aspiration or manual displacement. Added either serum free media for resting cells (mock) or serum free media with 100 ng/ml EGF. Used 100 μl of resting/activation media per well. Allowed incubation at 37° C. for 7.5 minutes. Removed activation or stimulation media manually or by aspiration. Immediately fixed cells with 4% formaldehyde in 1×PBS. Allowed incubation on bench top for 20 minutes at RT with no shaking. Washed five times with 1×PBS containing 0.1% Triton X-100 for 5 minutes per Wash. Removed Fixing Solution. Using a multi-channel pipettor, added 200 μl of Triton Washing Solution (1×PBS+0.1% Triton X-100). Allowed wash to shake on a rotator for 5 minutes at room temperature. Repeated washing steps 4 more times after removing wash manually. Using a multi-channel pipettor, blocked cells/wells by adding 100 μl of LI-COR Odyssey Blocking Buffer to each well. Allowed blocking for 90 minutes at RT with moderate shaking on a rotator. Added the two primary antibodies into a tube containing Odyssey Blocking Buffer. Mixed the primary antibody solution well before addition to wells (Phospho-EGFR Tyr1045, (Rabbit; 1:100 dilution; Cell Signaling Technology, 2237; Total EGFR, Mouse; 1:500 dilution; Biosource International, AHR5062). Removed blocking buffer from the blocking step and added 40 μl of the desired primary antibody or antibodies in Odyssey Blocking Buffer to cover the bottom of each well. Added 100 μl of Odyssey Blocking Buffer only to control wells. Incubated with primary antibody overnight with gentle shaking at RT. Washed the plate five times with 1×PBS+0.1% Tween-20 for 5 minutes at RT with gentle shaking, using a generous amount of buffer. Using a multi-channel pipettor added 200 μl of Tween Washing Solution. Allowed wash to shake on a rotator for 5 minutes at RT. Repeated washing steps 4 more times. Diluted the fluorescently labeled secondary antibody in Odyssey Blocking Buffer (Goat anti-mouse IRDye™ 680 (1:200 dilution; LI-COR Cat.#926-32220) Goat anti-rabbit IRDye™ 800CW (1:800 dilution; LI-COR Cat.#926-32211). Mixed the antibody solutions well and added 40 μl of the secondary antibody solution to each well. Incubated for 60 minutes with gentle shaking at RT. Protected plate from light during incubation. Washed the plate five times with 1×PBS+0.1% Tween-20 for 5 minutes at RT with gentle shaking, using a generous amount of buffer. Using a multi-channel pipettor added 200 μl of Tween Washing Solution. Allowed wash to shake on a rotator for 5 minutes at RT. Repeated washing steps 4 more times. After final wash, removed wash solution completely from wells. Turned the plate upside down and tap or blot gently on paper towels to remove traces of wash buffer. Scanned the plate with detection in both the 700 and 800 channels using the Odyssey Infrared Imaging System (700 nm detection for IRDye™ 680 antibody and 800 nm detection for IRDye™ 800CW antibody). Determined the ratio of total to phosphorylated protein (700/800) using Odyssey software and plot the results in Graphpad Prism (V4.0a). Data were plotted using GraphPad Prism (v4.0a) and IC50's calculated using a sigmoidal dose response curve fitting algorithm.

(c) An In Vitro Assay which Determines the Ability of a Test Compound to Inhibit HDAC Enzymatic Activity.

HDAC inhibitors were screened using an HDAC fluorimetric assay kit (AK-500, Biomol, Plymouth Meeting, Pa.). Test compounds were dissolved in dimethylsulphoxide (DMSO) to give a 20 mM working stock concentration. Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm. Each assay was setup as follows: Defrosted all kit components and kept on ice until use. Diluted HeLa nuclear extract 1:29 in Assay Buffer (50 mM Tris/Cl, pH 8.0, 137 mM NaCl, 2.7 mM KCl, 1 mM MgCl2). Prepared dilutions of Trichostatin A (TSA, positive control) and tested compounds in assay buffer (5× of final concentration). Diluted Fluor de Lys™ Substrate in assay buffer to 100 uM (50 fold=2× final). Diluted Fluor de Lys™ developer concentrate 20-fold (e.g. 50 μl plus 950 μl Assay Buffer) in cold assay buffer. Second, diluted the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Added Assay buffer, diluted trichostatin A or test inhibitor to appropriate wells of the microtiter plate. Added diluted HeLa extract or other HDAC sample to all wells except for negative controls. Allowed diluted Fluor de Lys™ Substrate and the samples in the microtiter plate to equilibrate to assay temperature (e.g. 25 or 37° C. Initiated HDAC reactions by adding diluted substrate (25 μl) to each well and mixing thoroughly. Allowed HDAC reactions to proceed for 1 hour and then stopped them by addition of Fluor de Lys™ Developer (50 μl). Incubated plate at room temperature (25° C.) for 10-15 min. Read samples in a microtiter-plate reading fluorimeter capable of excitation at a wavelength in the range 350-380 nm and detection of emitted light in the range 440-460 nm.

The following TABLE 1-B lists compounds representative of the invention and their activity in HDAC and EGFR assays. In these assays, the following grading was used: I≧10 μM, 10 μM>II>1 μM, 1 μM>III>0.1 μM, and IV≦0.1 μM for IC₅₀.

TABLE 1-B

| Compound | | | | |
|---|---|---|---|---|
| No. | HDAC | EGFR | HER2/ErB2 | VEGFR |
| 1 | I | IV | | |
| 2 | I | IV | | |
| 3 | I | IV | | |
| 4 | III | IV | | |
| 5 | IV | IV | | |
| 6 | IV | IV | IV | III |
| 7 | I | IV | IV | |
| 8 | I | IV | | |
| 9 | III | IV | | |
| 10 | III | IV | | |
| 11 | IV | IV | III | |
| 12 | IV | IV | III | |
| 13 | I | IV | | |
| 14 | II | IV | | |
| 15 | IV | III | | |
| 16 | III | IV | | |
| 17 | IV | IV | | |
| 18 | IV | IV | III | |
| 19 | I | IV | | |
| 21 | II | III | | |
| 22 | IV | IV | | |
| 23 | IV | III | | |
| 24 | IV | III | | |
| 30 | IV | IV | III | |
| 36 | IV | IV | | |
| 38 | II | IV | | |
| 40 | IV | IV | III | |
| 42 | III | IV | | |
| 43 | III | IV | | |
| 44 | IV | IV | III | |
| 45 | I | III | | |
| 50 | III | III | | |
| 63 | III | II | | |
| 66 | III | IV | | |
| 68 | II | IV | | |
| 69 | III | IV | | |
| 70 | IV | IV | IV | |
| 75 | IV | IV | III | |
| 76 | IV | IV | III | |
| 77 | IV | IV | | |
| 78 | IV | III | | |
| 79 | IV | IV | III | I |
| 80 | IV | II | | |
| 81 | III | III | | |
| 82 | III | III | | |
| 83 | IV | I | | |
| 84 | IV | III | | |
| 85 | IV | IV | III | II |
| 86 | IV | III | | |
| 87 | IV | III | | |
| 88 | IV | IV | II | |
| 89 | IV | III | | |
| 90 | IV | N/A | | |
| 91 | II | IV | | |
| 92 | III | IV | | |
| 93 | II | IV | | |
| 94 | I | IV | | |
| 102 | | IV | | |
| 103 | I | | | |
| 107 | III | | | |
| 112 | I | | | |
| 118 | II | | | |
| 121 | I | | | |
| 124 | I | | | |
| 125 | III | | | |
| 138 | II | | | |
| 139 | II | | | |
| 144 | III | | | |
| 145 | IV | IV | IV | |
| 151 | IV | IV | IV | |
| 155 | IV | IV | IV | |
| 161 | IV | | III | |
| 162 | IV | IV | III | |
| 167 | IV | IV | III | |
| 168 | IV | IV | III | |
| 174 | I | | | |
| 177 | III | IV | IV | |
| 178 | III | | | |
| 198 | IV | IV | IV | |
| 199 | IV | IV | IV | |
| 200 | IV | IV | IV | |
| 201 | III | IV | IV | |
| 202 | III | | | |
| 203 | III | | | |
| 204 | II | | | |
| 205 | II | | | |
| 206 | IV | IV | IV | |
| 207 | IV | IV | IV | |
| 208 | IV | | | |
| 209 | IV | | | |

A representative number of compounds were assayed against several different cell lines using the cell proliferation assay:

### Cell Proliferation Assay:

Cancer cell lines were plated at 5,000 to 10,000 per well in 96-well flatted bottomed plates with various concentration of compounds. The cells were incubated with compounds for 72 hours in the presence of 0.5% of fetal bovine serum. Growth inhibition was accessed by adenosine triphosphate (ATP) content assay using Perkin Elmer ATPlite kit. ATPlite is an ATP monitoring system based on firefly luciferase. Briefly, 25 μl of mammalian cell lysis solution was added to 50 μl of phenol red-free culture medium per well to lyse the cells and stabilize the ATP. 25 μl of substrate solution was then added to the well and subsequently the luminescence was measured.

The results are presented below in TABLE C. In these assays, the following grading was used: I≧10 μM, 10 μM>II>1 μM, 1 μM>III>0.1 μM, and IV≦0.1 μM for IC₅₀.

TABLE C

| | Compound No. | | | | | | | | | |
|---|---|---|---|---|---|---|---|---|---|---|
| Cell Line | 6 | 12 | 18 | 40 | 44 | 66 | 70 | 77 | 79 | 85 |
| Breast_MCF7 | | IV | | | | | | | | |
| Breast_MDAMB468 | | IV | | | | | | | | |
| Breast_SkBr3 | | III | | | | | | | | |
| Colon_HCT116 | | III | III | | | | | | | III |
| Epidermoid_A431 | | III | | | | | | | | |
| Lung_H1703 | III | III | | | II | | | | | |
| Lung_H1975 | III | III | | | II | | | | | |
| Lung_H2122 | III | III | | | II | | | | | |
| Lung_H292 | | IV | | | | | | | | |
| Lung_H358 | III | III | | | II | | | | | |
| Lung_H460 | III | III | | | II | | | | | |
| Lung_HCC827 | IV | III | | | III | | | | | |
| Pancreas_BxPC3 | III | IV | III | II | II | II | II | II | II | III |
| Pancreas_Capan1 | II | III | | | II | | | | | |
| Pancreas_CFPAC | | III | III | II | | I | II | II | II | II |
| Pancreas_HPAC | | II | II | II | | II | I | I | I | II |
| Pancreas_MiaPaCa2 | | III | III | II | | II | II | III | II | II |
| Pancreas_PANC1 | | III | III | II | | II | II | I | II | II |
| Prostate_22RV1 | | III | | | | | | | | |
| Prostate_PC3 | III | III | | | | | | | | |

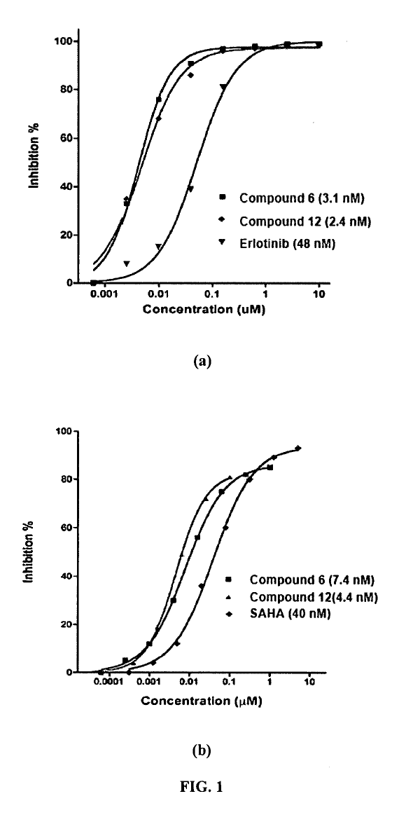

FIG. 1 shows that compounds of the invention, such as compounds 6 and 12 are more active than erlotinib and SAHA in EFGR enzyme assay and HDAC enzyme assay. In the EFGR enzyme assay, compounds of the invention is more potent than erlotinib by approximately 15-20 fold. In the HDAC enzyme assay, compounds of the invention is more potent than SAHA by approximately 5-10 fold.

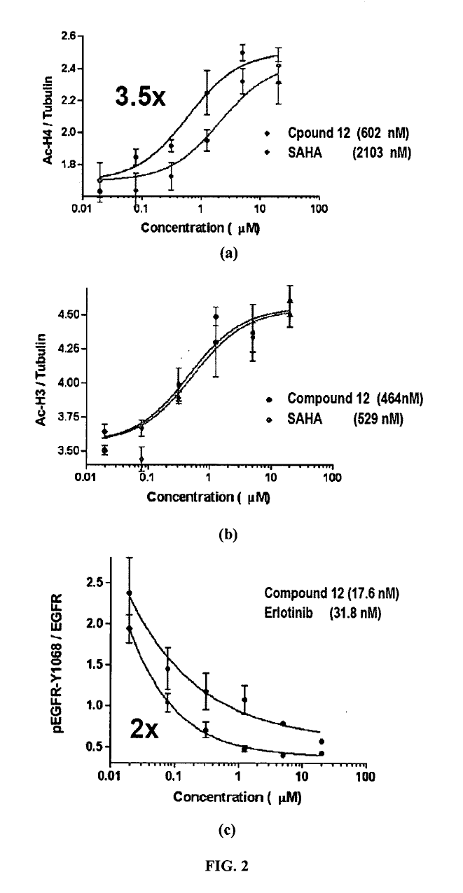

FIG. 2 illustrates the improvement in inhibition of histone acetylation and EGFR phosphorylation by compound 12 as compared with SAHA and Erlotinib respectively. Inhibition on both kinase (EGFR) and non-kinase (HDAC) cancer targets by a compound 12.

Table D illustrates the potency of compounds of the invention. For example, compound 12 is more active than Erlotinib and SAHA in various cancer cell lines (IC50 in μM). Cell lines from five major types of cancer (lung, breast, prostate, colon, and pancreas) responded better to compound 12 than a combination of erlotinib and SAHA. Surprisingly, the compounds of the invention are active against cell lines that are resistant to Tarceva® and Iressa®. In these assays, the following grading was used: D≧5 μM, 5 μM>C≧0.5 μM, 0.5 μM>B≧0.05 μM, and A≦0.05 μM for IC₅₀.

TABLE D

| | | | | Erlotinib/SAHA | Compound |
|---|---|---|---|---|---|
| Tumor Line | Tumor Type | SAHA | Erlotinib | Combined | 12 |
| MDA-MB-231 | Breast adenocarcinoma | B | D | B | A |
| HCT116 | Colon cancer | C | D | C | A |
| MCF-7 | Breast adenocarcinoma | C | D | C | A |
| MDA-MB-468 | Breast adenocarcinoma | C | C | C | A |
| SKBr3 | Breast carcinoma | C | D | C | B |
| PC-3 | Prostate adenocarcinoma | C | D | C | C |
| Caki-1 | Renal carcinoma | B | B | B | A |
| A431 | Epidermoid carcinoma | C | C | C | B |
| 22RV1 | Prostate carcinoma | B | B | B | B |

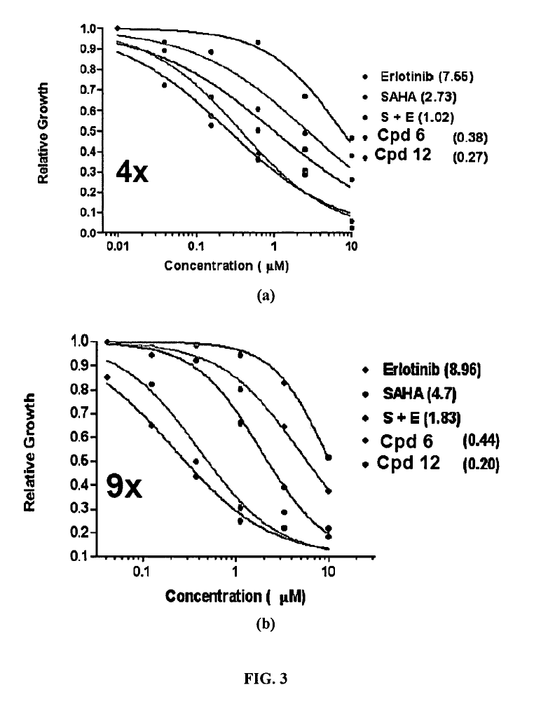

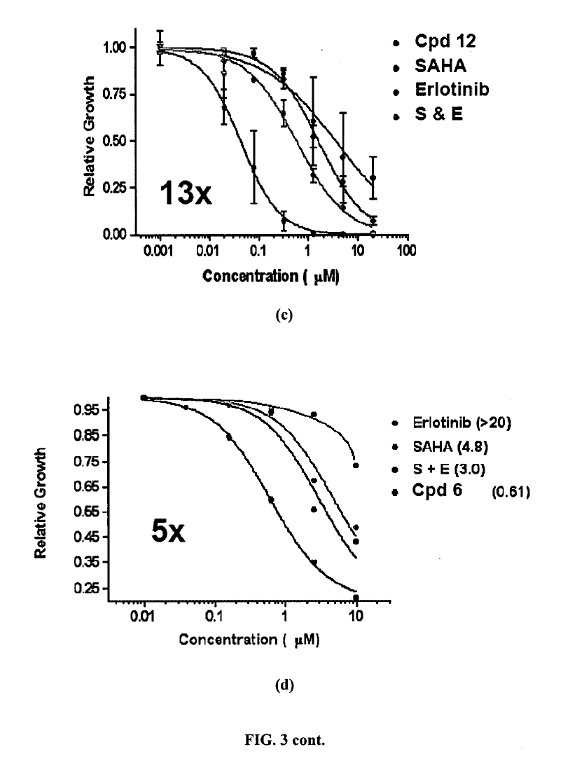

FIG. 3 shows examples of greater anti-proliferative activity against several different cancer cell lines. FIG. 3 further shows that compounds of the invention are more potent than SAHA alone, Erlotinib alone, and SAHA and Erlotinib combined.

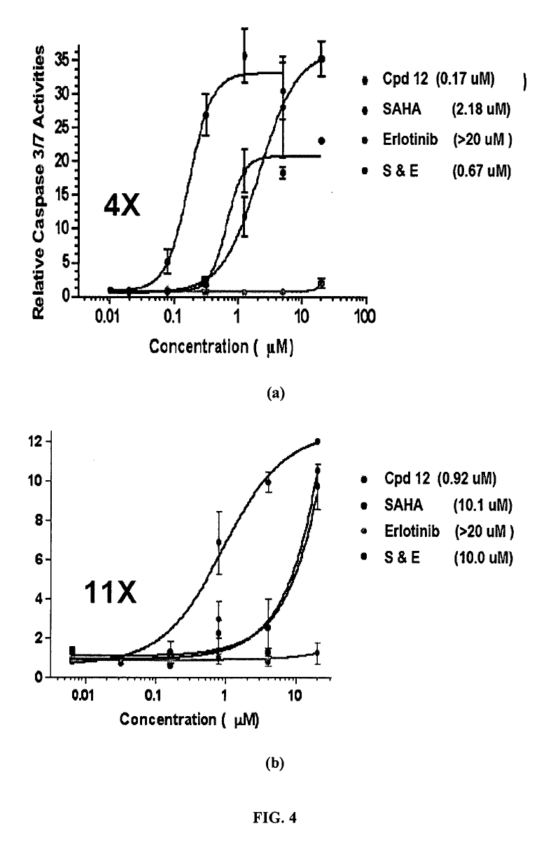

FIG. 4 displays the potency of compound 12 in induction of apoptosis in colon and breast cancer cells. Compound 12 induced approximately 4-11 times more cell apoptosis as measured by increased Caspase 3&7 activity. Erlotinib was inactive at a concentration <20 μM. The high potency displayed by compound 12 over Erlotinib suggests that compounds of the invention can be used to treat tumor cells that are resistant to Erlotinib.

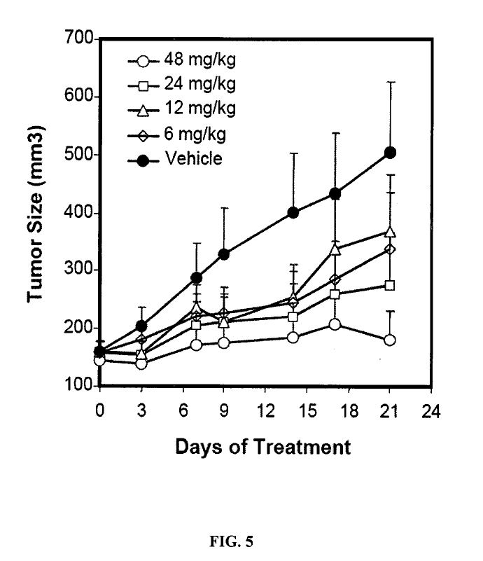

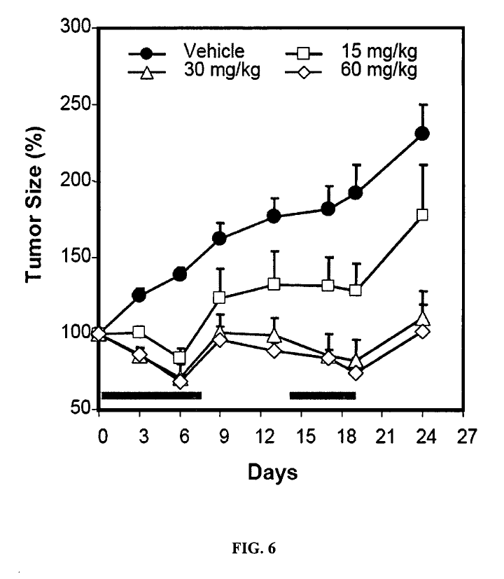

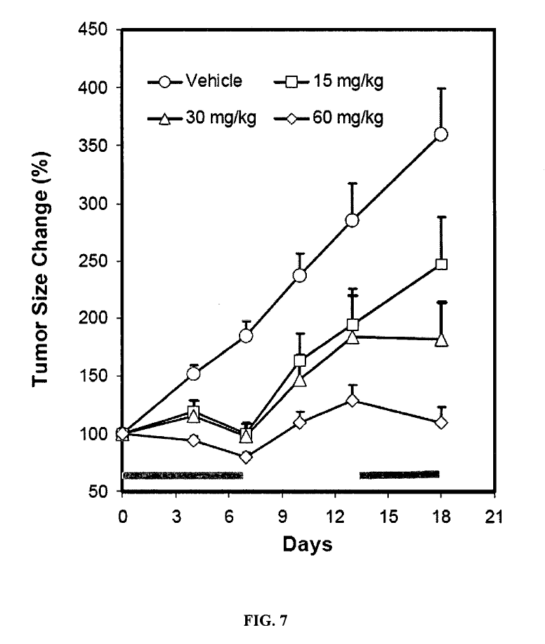

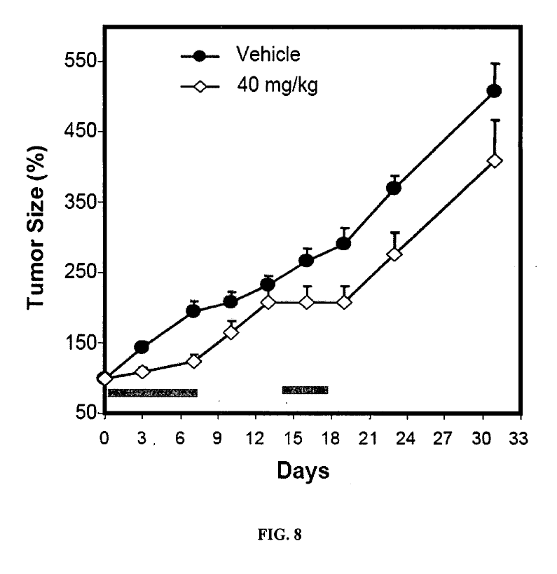

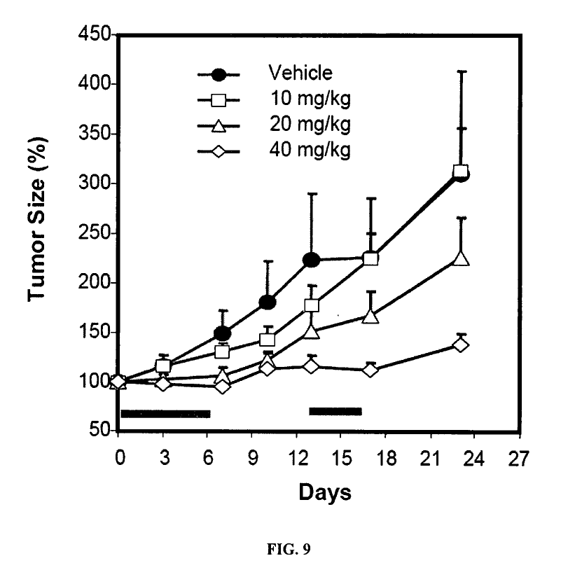

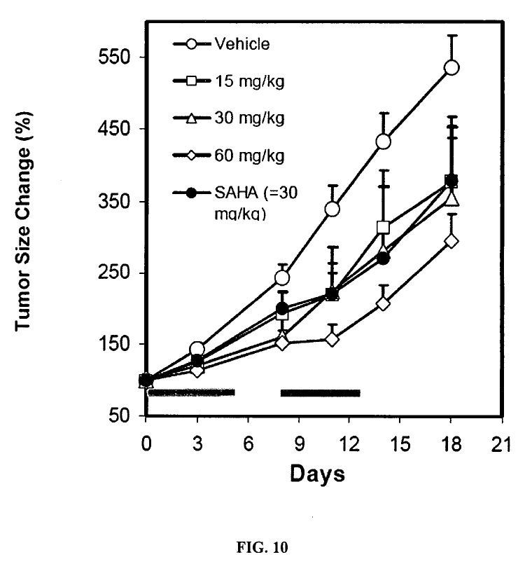

FIGS. 5-10 illustrate the efficacy of compound 12 in various tumor xenograft models. Table E below summarizes the in vivo experiments that were carried out to give results represented in FIGS. 5-10.

TABLE E

| | | | | Dosing | |
|---|---|---|---|---|---|
| | | | | regimen | Pre- |
| | | | Method of | (on-off- | treatment |
| Model | Cancer type | Dosage groups | administration | on) | tumor size |
| A431 | Epidermoid | vehicle | IP | Once | 156 ± 57 mm³ |
| | | 6 mg/kg | | daily for | |
| | | 12 mg/kg | | 21 days | |
| | | 24 mg/kg | | | |
| | | 48 mg/kg | | | |
| H358 | NSCLC | vehicle | IV - 2 min | 7-7-5 | 84 ± 23 mm³ |
| | | 15 mg/kg | infusion | | |
| | | 30 mg/kg | | | |
| | | 60 mg/kg | | | |
| H292 | NSCLC | vehicle | IV - 2 min | 7-7-5 | 116 ± 23 mm³ |
| | | 15 mg/kg | infusion | | |
| | | 30 mg/kg | | | |
| | | 60 mg/kg | | | |
| BxPC3 | Pancreatic | vehicle | IV - 2 min | 7-7-2 | 201 ± 53 mm³ |
| | | 10 mg/kg | infusion | | |
| | | 20 mg/kg | | | |
| | | 40 mg/kg | | | |
| PC3 | Prostate | vehicle | IV - 2 min | 7-7-5 | 195 ± 50 mm³ |
| | | 10 mg/kg | infusion | | |
| | | 20 mg/kg | | | |
| | | 40 mg/kg | | | |
| HCT116 | Colon | vehicle | IV - 2 min | 5-2-5 | 91 ± 23 mm³ |
| | | 15 mg/kg | infusion | | |
| | | 30 mg/kg | | | |
| | | 60 mg/kg | | | |
| | | SAHA 20 mg/kg | | | |
| HCC827 | NSCLC | vehicle | IV - 2 min | Once | 149 ± 36 mm³ |
| (apoptosis/ | | 30 mg/kg | infusion | daily for | |
| anti- | | | | 3 days | |
| proliferation) | | | | | |
| BxPC3 | Pancreatic | 60 mg/kg | IV - 2 min | Single IV | NA |
| (apoptosis/ | | | infusion | infusion | |
| anti- | | | | | |
| proliferation) | | | | | |

A representative protocol for the in vivo experiment is as followed:

1-10×10⁶ human cancer cells were implanted subcutaneously to the athymic (nu/nu) mice. When the tumors reached about 100 mm³ in volume, the mice were treated with the compound by tail vein infusion. Routinely 5 groups (8-12 mice per group) are needed for a typical efficacy study, including one negative control, one positive control, and three testing groups for 3 dose levels of the same compound. Usually a 7-7-5 (on-off-on) regimen was used for one typical study. The tumor size was measured with an electronic caliper and body weight measured with a scale twice weekly. The tumors were removed from euthanized mice at the end of the study. One half of each tumor was frozen in dry ice and stored at −80° C. for PK or Western blot analysis. The other half was fixed with formalin. The fixed tissues were processed, embedded in paraffin and sectioned for immunohistochemistry staining.

### Protocol for Radioisotope Assay for HER2

10 nM HER2 and 0.1 mg/ml polyEY were placed in the reaction buffer and 2 mM MnCl₂, 1 μM ATP and 1% DMSO final were added. The reaction mixture was incubated for 2 hours at room temperature. The conversion rate of ATP was 22%.

HER2 (Accession number: GenBank X03363) is characterized as follows: N-terminal GST-tagged, recombinant, human HER2 amino acids 679-1255, expressed by baculovirus in Sj9 insect cells. Purity>90% by SDS PAGE and Coomassie blue staining. MW=91.6 kDa. Specific Activity of 40 U/mg, where one unit of activity is defined as 1 nmol phosphate incorporated into 30 ug/ml Poly (Glu:Tyr)4:1 substrate per minute at 30° C. with a final ATP concentration of 100 μM. Enzyme is in 25 mM Tris-HCl, pH 8.0, 100 mM NaCl, 0.05% Tween-20, 50% glycerol, 10 mM reduced glutathione, and 3 mM DTT.

### REFERENCES

1. Meyer, M. et. al., EMBO J. 18, 363-374 (1999)
2. Rahimi, N. et. al., J. Biol Chem 275, 16986-16992 (2000)

Compounds of the invention are found to be active against various kinases. For example, Table F shows inhibition of compound 12 in a panel of kinase assays. Furthermore, Compound 12 is much more active than Erlotinib in Her-2 assay.

TABLE F

| Assays | Concentrations (μM) | Inhibition (%) |
|---|---|---|
| Abl Kinase | 5 | 57 |
| FGFR2 Kinase | 5 | 73 |
| FLT-3 Kinase | 5 | 85 |
| VEGFR2 Kinase | 5 | 64 |
| Lck Kinase | 5 | 56 |
| Lyn Kinase | 5 | 95 |
| Ret Kinase | 5 | 93 |

Her-2
Compound 12 IC50 = 188 nM
Erlotinib IC50 = 1473 nM

### Example 73

### Preparation of Captisol Formulation of Compound 12

A. Preparation of 25, 30, 40, 50 and 60 mg/ml Solutions of Compound 12 in 30% Captisol

### (i) With Tartaric Acid

A 30% Captisol formulation was prepared by adding 2.7 ml water to a vial containing 0.9 g Captisol. The mixture was then mixed on a vortexer to give ˜3 ml of a clear solution.

In order to prepare a formulation of 25 mg/ml solution of compound 12, 1 ml of the 30% Captisol solution was added to a vial containing 25 mg of compound 12 and 8.6 mg tartaric acid and the resulting mixture was mixed on a vortexer or sonicated at 30° C. for 15 to 20 minutes to give a clear yellowish solution. The resulting solution is stable at room temperature.

In order to prepare a formulation of 30 mg/ml solution of compound 12, 1 ml of the 30% Captisol solution was added to a vial containing 30 mg of compound 12 and 10.4 mg tartaric acid (1.0 eq) at room temperature.

In order to prepare a formulation of 40 mg/ml solution of compound 12, 1 ml of the 30% Captisol solution was added to a vial containing 40 mg of compound 12 and 17.9 mg tartaric acid (1.3 eq) at 36° C.

In order to prepare a formulation of 50 mg/ml compound 12 in Captisol, 1 ml of 30% Captisol was added to 50 mg compound 12, 22.5 mg tartaric acid (1.3 eq) at 37° C.

In order to prepare a formulation of 60 mg/ml compound 12 in Captisol, the 30% Captisol was added to a vial containing 60 mg compound 12 and 26.9 mg tartaric acid (1.3 eq) at 36° C. The solution was diluted in 1× water and 2×D5W. The diluted solution is stable at room temperature for >12 h.

### (ii) With Citric Acid

In order to prepare a formulation of 25 mg/ml solution of compound 12, 1 ml of the 30% Captisol solution was added to a vial containing 25 mg of compound 12 and 11.1 mg citric acid (1.0 eq) and the resulting mixture was mixed on a vortexer or sonicated at room temperature for 15 to 20 minutes to give a clear yellowish solution.

(iii) With Hydrochloric Acid

In order to prepare a formulation of 25 mg/ml solution of compound 12, 1 ml of the 30% Captisol solution was added to a vial containing 25 mg of compound 12 and 57.5 μl hydrochloric acid (1.0 eq) and the resulting mixture was mixed on a vortexer or sonicated at room temperature for 15 to 20 minutes to give a clear yellowish solution.

(iiii) With Sodium Salt

In order to prepare a formulation of 7.5 mg/ml solution of compound 12, 1 ml of the 30% Captisol solution was added to a vial containing 7.5 mg of compound 12 sodium salt and the resulting mixture was mixed on a vortexer or sonicated at room temperature for 15 to 20 minutes to give a clear yellowish solution.

### B. Filtration of the Solution

The formulations of compound 12 from A (i) was filtered through a 0.2-μm presterilized filter with >98% recovery.

### C. Preparation of a Lyophilisate

The formulations of compound 12 (25 mg/ml) from A (i) and A (iii) were lyophilized to form lyophilisate as a yellow powder.

The lyophilisate resulted from A (i) formulation was chemically stable at following temperatures, −20° C., room temperature, and 40° C. for at least 2 weeks. It can be stored at 4° C. for greater than 2 weeks without decomposition. The lyophilisate resulted from A (iii) was stable at −20° C. for at least two weeks.

### D. Dilution Study

The formulations of compound 12 from A (i) were diluted with D5W (10-, 20-, and 50-fold) and were chemically stable and remained in solution without precipitation (>48 hours).

The formulations of compound 12 from A (ii), A (iii) and A (iiii) were diluted with D5W (10-fold) and remained in solution without precipitation (>12 hours).

### Example 74

### Characteristics of Sodium Hydrochloride Citric Acid and Tartaric Acid Salts or Complexes of Compound 12 Formulated in CAPTISOL

Sodium, hydrochloride, citrate and tartrate salts of a test compound of Formula I were prepared in 30% CAPTISOL solutions and were studied for the following:

Table G shows the physiochemical as well as pharmacokinetic (PK) and pharmacodynamic (PD) properties of sodium, hydrochloride, citric acid and tartaric acid salts of Compound 12.

TABLE G

| | Sodium | HCl | Citric Acid | Tartaric Acid |
|---|---|---|---|---|
| Solubility | 7.5 mg/ml | 25 mg/ml | 25 mg/ml | 60 mg/ml |
| pH | 10-11 | 2-3 | 4-5 | 3-4 |
| IV Tissue | High | High | Low | High |
| Dilution with | >10x | >10x | >10x | >50x |
| D5W | | | | |
| Chemical stability | >12 h | >12 h | >12 h | >12 h |
| in diluted solution | | | | |
| 2-week chemical | ND | −20° C. | ND | −20° C., RT, |
| stability in | | | | 40° C. |
| lyophilisate | | | | |
| Deliverable | 220-250 mg | >750 mg | >750 mg | >1800 mg |
| highest daily dose | | | | |
| in humans | | | | |

### Example 75

### Comparison of Anti-Tumor Activity of Composition of Compound 12 in 30% CAPTISOL and Erlotinib, a Prototype EGFRi in A549 NSCLC Xenograft Model

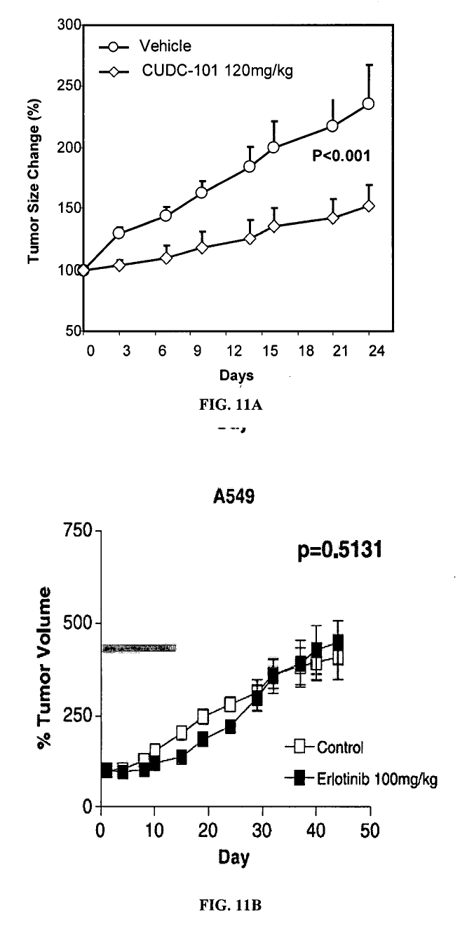

Administration of compound 12 in 30% CAPTISOL attenuated tumor growth in the NSCLC xenograft model. As shown in FIG. 11A, after 24 hours, animals treated with compound 12 showed a 150% increase in tumor size whereas animals treated with vehicle showed about a 240% change in tumor size. As shown in FIG. 11B, treatment of animals with Erolotinib did not significantly affect tumor size as compared to control.

### Example 76

### Effect of Composition of Compound 12 in 30% Captisol in HPAC Pancreatic Cancer Cells

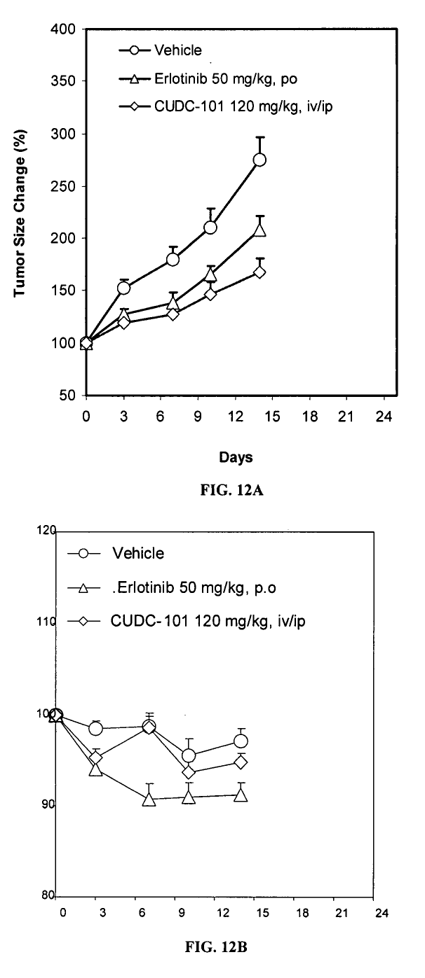

120 mg/kg of compound 12 in 30% CAPTISOL, 50 mg/kg erlotinib or vehicle were administered to animals daily and change in tumor size over time (days) was measured. As shown in FIG. 12A, administration of 120 mg/kg compound 12 in 30% CAPTISOL (iv/ip) resulted in greater attenuation of tumor growth than either erlotinib (po) or vehicle.

### Pharmacokinetic Studies in Mice, Rats, and Dogs

The experimental methods used for Examples 77-83 are described below.

Animals: Mice (CD-1, male, 25-30 g), rats (Spraque Dawle, 260-300 g) and dogs (Beagles, male, 9-11 kg) were used for the PK studies. Animals were provided pelleted food and water ad libitum and kept in a room conditioned at 23±1° C., humidity of 50-70%, and a 12-hour light/12-hour dark cycle.

Drug Preparation and Administration. Compound 12 was dissolved in 30% CAPTISOL with equal molar concentration of tartaric acid or HCl or citric acid, or NaOH. Compound was administered via an intravenous (iv) infusion. Conditions for iv infusion for each animal are shown below:

Mouse: 20 mg/kg and 60 mg/kg for 2 min i.v. infusion

Rat: 20 mg/kg for 5 min i.v. infusion

Beagle: 25 mg/kg for 30 min i.v. infusion.

Blood and tissue Sample Collection. Blood was collected into tubes containing sodium heparin anticoagulant at various time points. The plasma was separated via centrifugation and stored in −40° C. before analysis.

Plasma Sample Extraction. Plasma samples were prepared by protein precipitation. An internal standard was added into plasma samples. A 50 μl of plasma was combined with 150 μl of acetonitrile, vortexed, and centrifuged for 10 min at 10000 rpm. The supernatant was then injected onto LC/MS/MS.

Samples were compared to standards made in plasma. These standards were prepared by serial dilution. An internal standard was added into the plasma with standard.

Tissue Sample Extraction. Lung and colon samples (20-200 mg) were used for extraction. Tissues were homogenized in 0.8 ml water. An internal standard was added into the tissue homogenates. The homogenates were extracted with 1-ml ethyl acetate for three times. After evaporation, the residual was reconstituted in 0.1 ml acetonitrile for LC/MS/MS assay.

LC/MS/MS Analytical Methods.

LC Conditions are shown below:

| LC Instrument: | Agilent HPLC 1100 Series | |
|---|---|---|
| Autosampler: | Agilent G1367A Autosampler | |
| Analytical Column: | YMC Pro C18 S3 (3μ, 2.0 * 50 mm, 120 Å) | |
| Guard Column: | YMC Pro C18 S3 Guard Column (3μ, 2.0 * | |
| | 10 mm, 120 Å) | |
| Column Temp: | in ambient | |
| Mobile Phase: | A: acetonitrile:water:formic acid | |
| | (5:95:0.1, v/v/v) | |
| | B: acetonitrile:water:formic acid | |
| | (95:5:0.1, v/v/v) | |
| LC Gradient Program | 0~1 min: | mobile phase A: 100% |
| | 1~2.5 min: | mobile phase A: 100% to 20% |
| | 2.5~3 min: | mobile phase A: 20% |
| | 3~4 min: | mobile phase A: 20% to 100% |

| | |
|---|---|
| Flow Rate: | 200 μl/min |
| Autosampler Temp: | in ambient |
| Injection Volumn: | 5 μl |

Mass Spectrometer conditions are shown below:

| | |
|---|---|
| Instrument: | PE Sciex API 3000 |
| Interface: | Turbo Ion Spray (TIS) |
| Polarity: | Positive Ion |
| Scan: | Multiple Reaction Monitoring (MRM) |

### Single or Multiple Dosing Toxicity Study in Mice and Rats

The experimental methods used for the toxicity study below are described as follows:

### Experiment Design:

1. Single dosing MTD in mice
a. CD-1 mice, male, 24-26 gram
b. Dosing at 0, 50, 100, 200, 400 mg/kg, iv infusion 2 min
c. 8 mice per group

2. Single dosing MTD in rats
a. Sprague Dawley, male and female, 240-260 gram
b. Dosing at 0, 25, 50, 100, 200 mg/kg, iv infusion 5 min
c. 6 rats per group (3 male and 3 female)

3. 7-day-multiple dosing MTD in mice
a. CD-1 mice, male, 24-26 gram
b. Dosing at 0, 50, 100, 200 mg/kg/d ip
c. 6 mice per group
d. Blood and organs will be collected 2 hr after last dosing on Day 7 for hematology

4. 7-day multiple dosing MTD in rats
a. Sprague Dawley, male and female, 220-250 gram
b. Dose, 25, 50, 100, 200 mg/kg/d) iv infusion 5 min
c. 6 per group (3 male and 3 female)
d. Blood and organs will be collected 2 hr after last dosing on Day 7 for hematology

### Compound Preparation

The compound was dissolved in 30% Captisol with equal molar concentration of tartaric acid. The stock solution: 25 mg/ml Tartaric form in 30% Captisol, 1 ml/vial store at −40° C. For example, 1000 mg compound, 345 mg tartaric acid (0.345 mg tartaric acid per mg compound) and 40 ml 30% Captisol or 1000 mg compound, 2.3 ml 1N HCl (2.3 ul 1N HCl per mg compound), 12 gram Captisol, Add water to 40 ml. Stock solution is diluted with 30% CAPTISOL before use.

### Example 77

### Pharmacokinetics of Test Salts in Plasma, Lung and Colon after Intravenous Administration

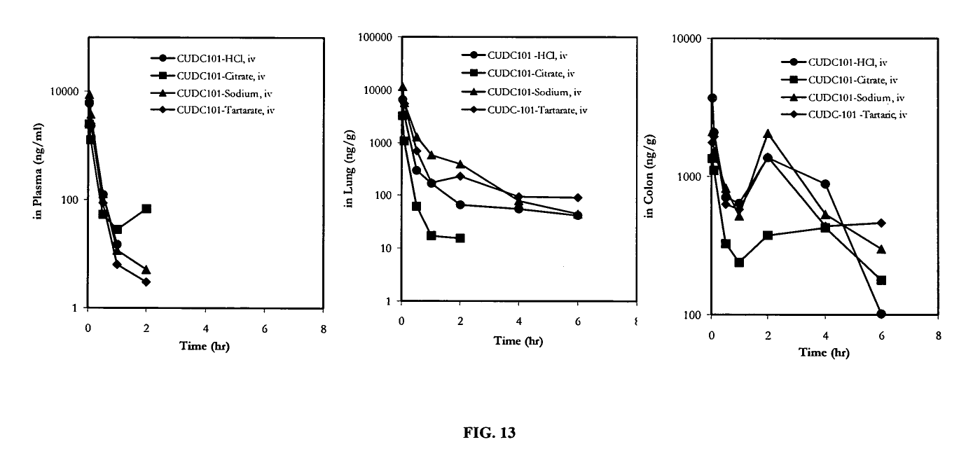

20 mg/kg of hydrochloride, citrate, sodium and tartrate salts of compound 12 in 30% CAPTISOL was administered intravenously to mice in order to determine the concentration (ng/ml) over time (hours) of compound 12 after intravenous (iv) administration in plasma, lung and colon. The results of these studies are shown in FIG. 13. As shown there, similar plasma and tissue pharmacokinetics was observed for the sodium, hydrochloride and tartrate salts.

### Example 78

### Pharmacokinetic Study of Compound 12 Formulation in Mice

20 mg/kg and 60 mg/kg of a hydrochloride salt of compound 12 in 30% CAPTISOL was administered intravenously (iv) and intraperitoneally (ip) to mice and the half life (t½), maximal observed concentration (Cmax) and area under the curve (AUC) were determined. As shown in Table H below, the concentration of compound 12 is dose proportional when administered intravenously but not intraperitoneally. The half-life of compound 12 in tissue is greater than that in plasma.

TABLE H

| | Plasma | | Lung | | Colon | |
|---|---|---|---|---|---|---|
| | 20 mgkg | 60 mg/kg | 20 mgkg | 60 mg/kg | 20 mgkg | 60 mg/kg |
| IV Dose | | | | | | |
| T ½ (hr) | 0.2 | 0.3 | 3.9 | 1.9 | 1.7 | 2.2 |
| Cmax (uM) | 27.7 | 61.7 | 15.2 | 96.9 | 8.5 | 29.4 |
| AUC (h * ng/ml) | 715 | 3124 | 1571 | 8313 | 5529 | 13473 |
| IP Dose | | | | | | |
| T ½ (hr) | 0.26 | 0.51 | 2.2 | 3.5 | NA | NA |
| Cmax (uM) | 8.5 | 14.4 | 7.8 | 11.6 | NA | NA |
| AUC (h * ng/ml) | 3751 | 5721 | 4433 | 8309 | NA | NA |

### Example 79

### Pharmacokinetic Study of Compound 12 Formulation in Rats

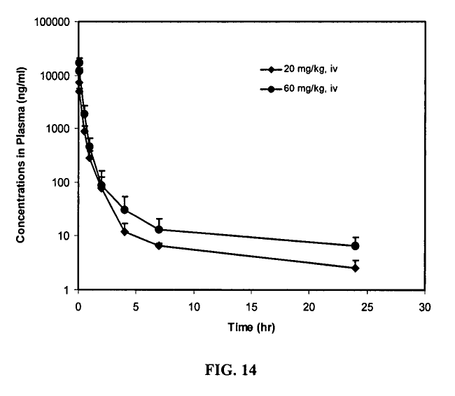

20 mg/kg and 60 mg/kg of a hydrochloride salt of compound 12 in 30% CAPTISOL was administered (iv) to rats and the concentration (ng/ml) of the compound was measured in plasma over thirty hours. As shown in FIG. 14, the concentration of compound 12 in the plasma of the rat was proportional to the dose of compound 12 administered.

### Example 80

### Single Dose IV Toxicity Study in Mice with the Compound 12 Formulation

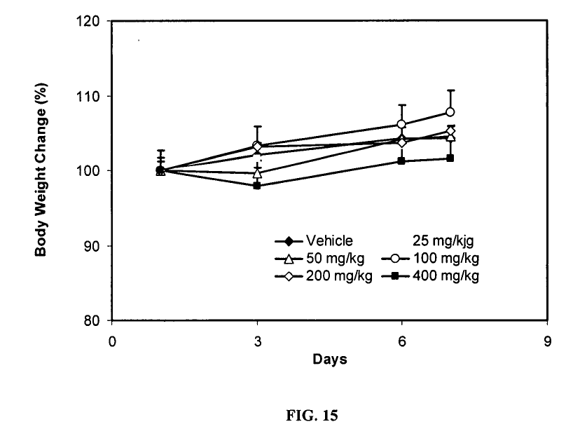

A single dose of compound 12 (25, 50, 100, 200 or 400 mg/kg) in 30% CAPTISOL was administered (iv) to mice and change in body weight was measured over nine day to assess toxicity of the various doses of compound 12. As shown in FIG. 15, administration of up to 200 mg/kg of compound 12 did not result in a significant change in body weight.

### Example 81

### Seven Day Repeat IP Toxicity Study in Mice Using Compound 12 Formulation

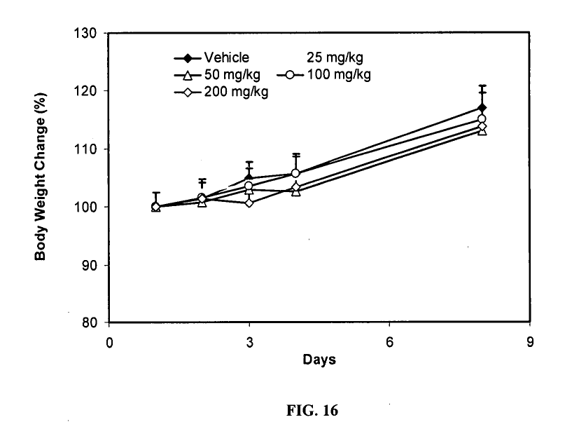

Repeated dosing of compound 12 over seven days (25, 50, 100, 200 or 400 mg/kg) in 30% CAPTISOL was administered (ip) to mice and change in body weight was measured over seven days. As shown in FIG. 16, repeated administration of up to 100 mg/kg of compound 12 did not result in a significant change in body weight.

### Example 82

### Single Does IV Toxicity Study in Rats Using Compound 12 Formulation

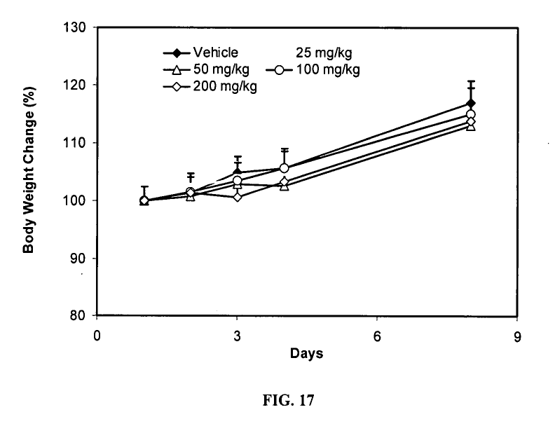

A single dose of compound 12 (25, 50, 100 or 200 mg/kg) in 30% CAPTISOL was administered (iv) to rats and change in body weight was measured over eight days to assess toxicity of the different doses of compound 12. As shown in FIG. 17, administration of up to 200 mg/kg did not result in a significant change in body weight.

TABLE 2-A

| | |
|---|---|
| | (III) |
| 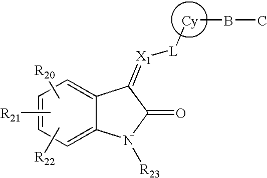 | |

SECTION 2:

| Com- | |
|---|---|
| pound | |
| No. | Structure |
| 1 | 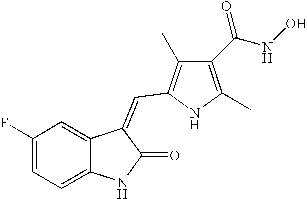 |
| 2 | 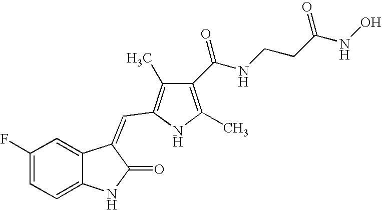 |
| 3 | 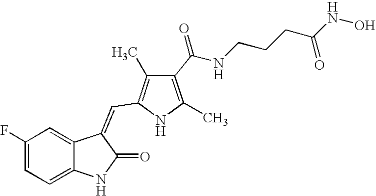 |
| 4 | 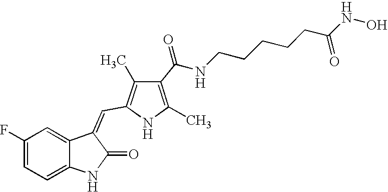 |
| 5 | 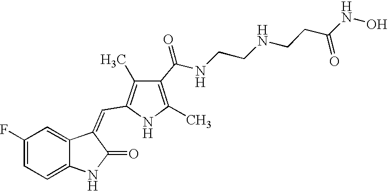 |
| 6 | 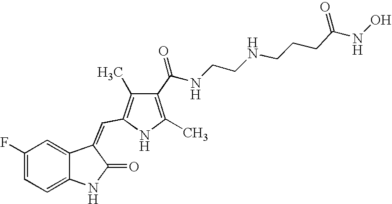 |
| 7 | 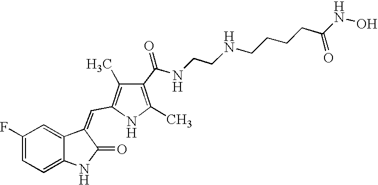 |
| 8 | 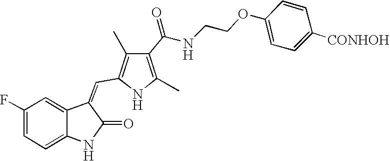 |
| 9 | 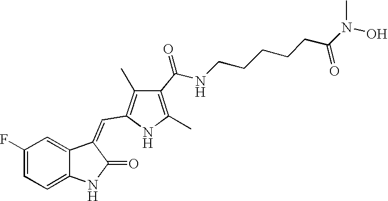 |
| 10 | 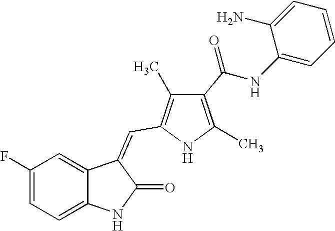 |
| 11 | 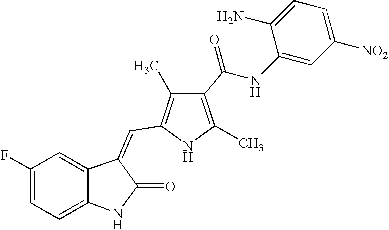 |
| 12 | 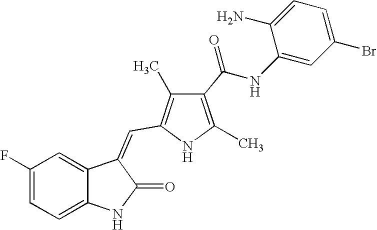 |
| 13 | 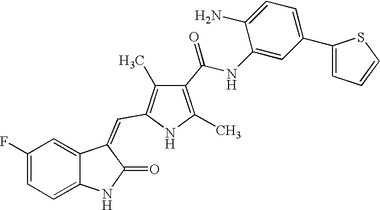 |
| 14 | 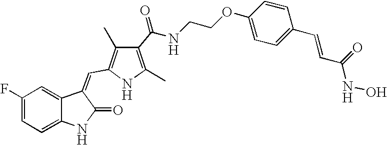 |
| 15 | 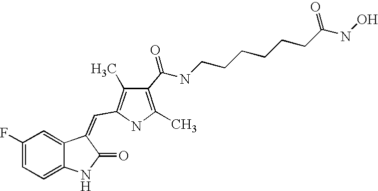 |
| 16 | 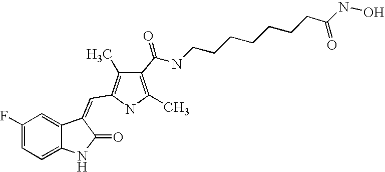 |
| 17 | 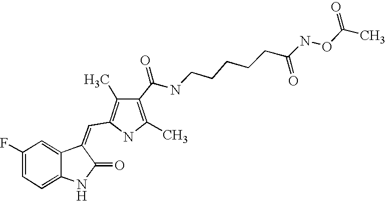 |
| 18 | 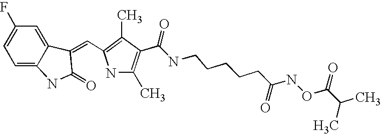 |
| 19 | 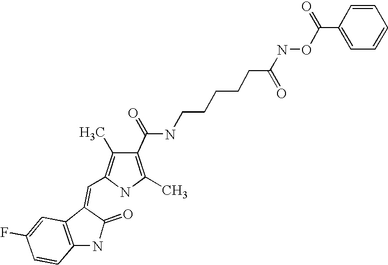 |
| 20 | 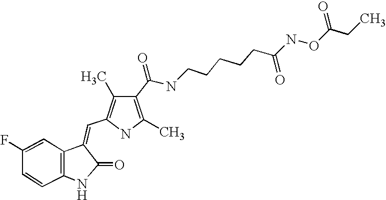 |
| 21 | 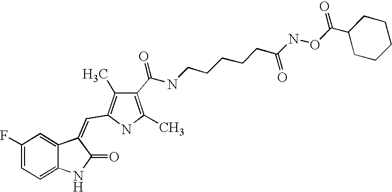 |
| 22 | 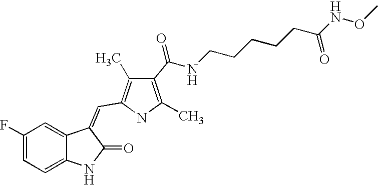 |
| 23 | 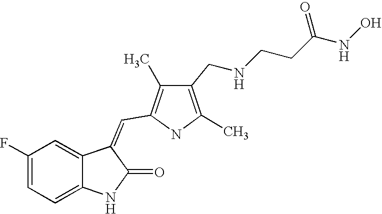 |
| 24 | 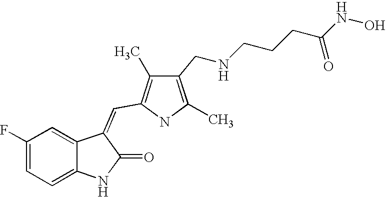 |
| 25 | 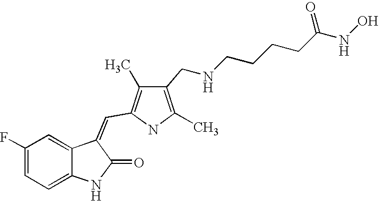 |
| 26 | 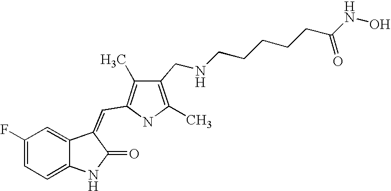 |
| 27 | 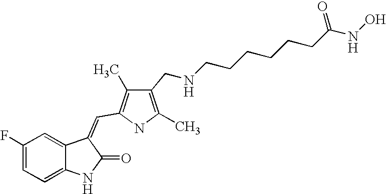 |
| 28 | 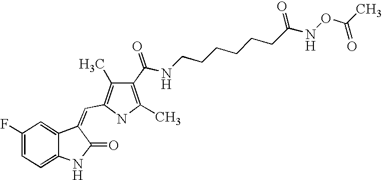 |
| 29 | 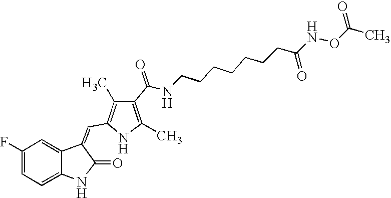 |

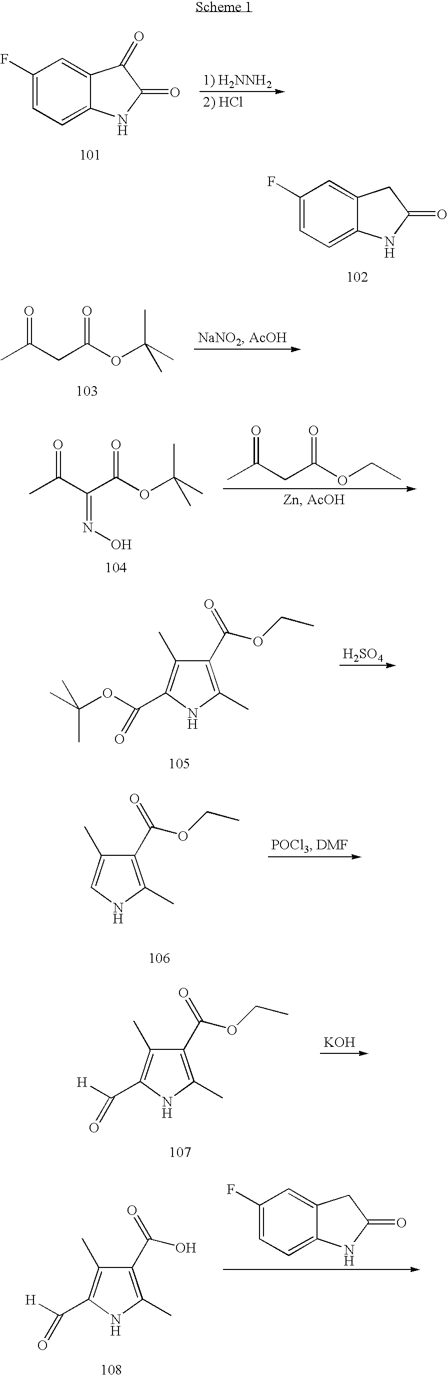

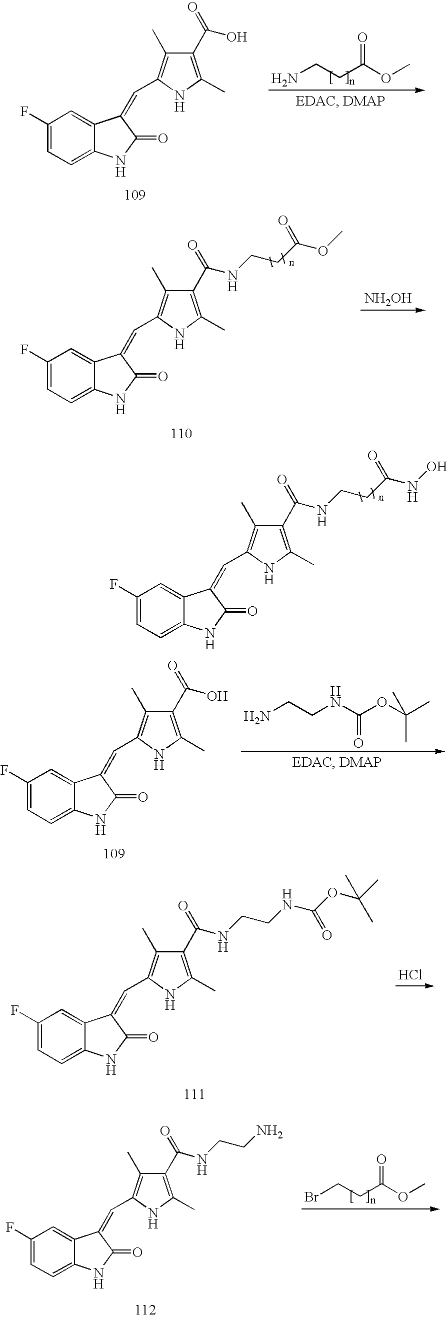

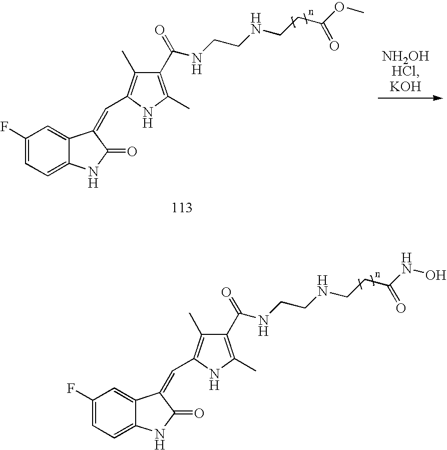

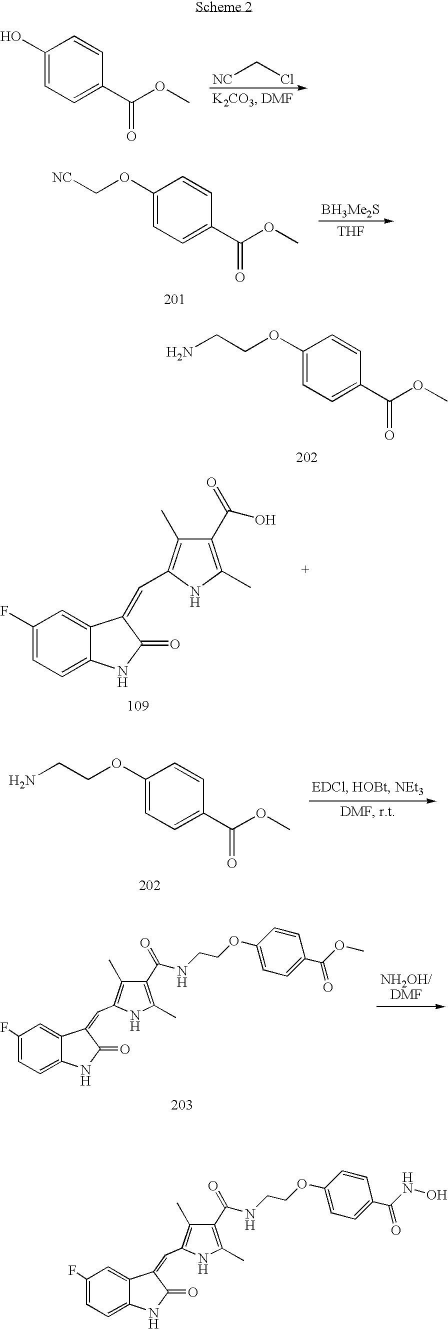

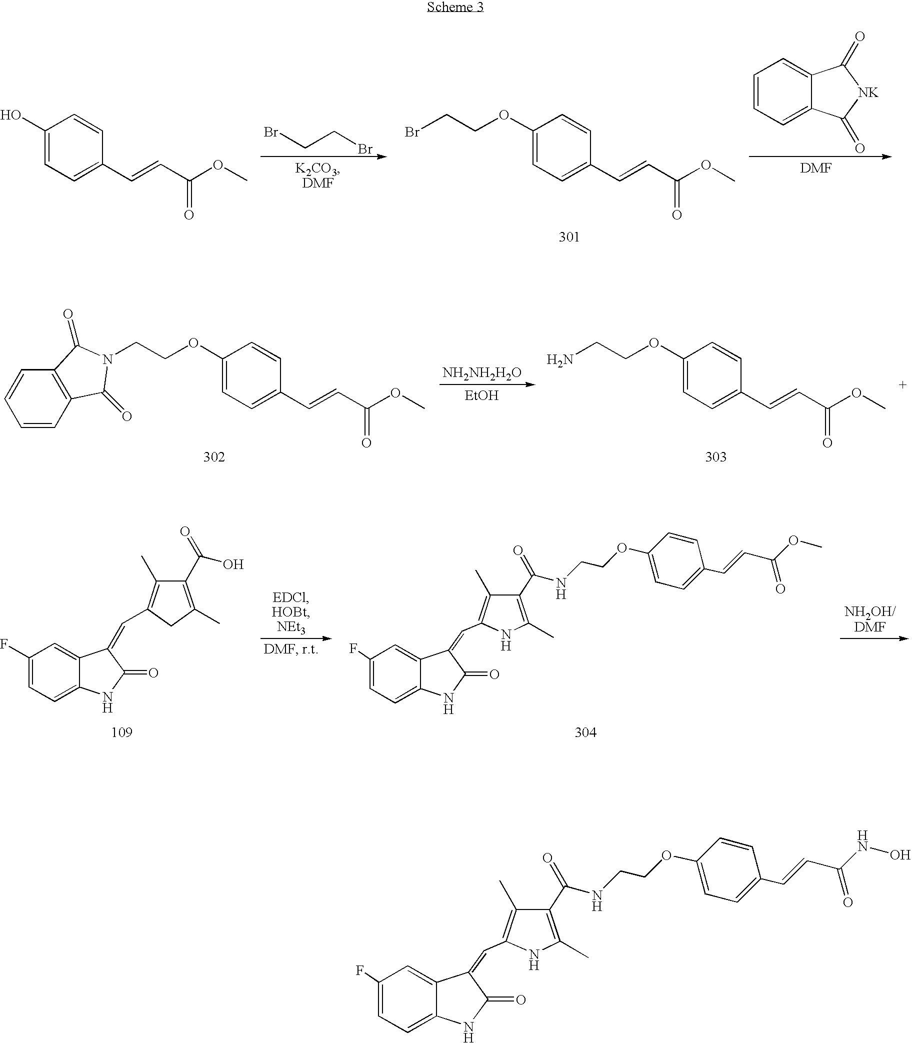

### Example 1

### Preparation of 5-((Z)-(5-fluoro-2-oxoindolin-3-ylidene)methyl)-N-hydroxy-2,4-dimethyl-1H-pyrrole-3-carboxamide (compound 1)

### Step 1a. 5-fluoroindolin-2-one (compound 102)

KOH (4.07 g, 73 mmol) was added into a mixture of compound 101 (6.0 g, 36 mmol), ethyleneglycol (95 mL) and hydrazine hydrate (2.6 g, 52 mmol). The reaction mixture was stirred at 80° C. for 3 h, and then was cooled to room temperature and was poured to ice cold water. The pH of the above mixture was adjusted to pH 1-2 with 12 N hydrochloric acid and the mixture was stirred at room temperature for 12 h. The mixture was then extracted with EtOAc. The organic phase was collected, evaporated to give yellow solid product 102 (4.5 g. 81.9%). LCMS: m/z 152 (M+1), ¹H NMR (DMSO-d₆) δ3.46 (s, 2H), 6.95 (m, 3H), 10.35 (s, 1H).

### Step 1b. tert-Butyl 2-(hydroxyimino)-3-oxobutanoate (compound 104)

To a solution of compound 103 (69.52 g, 44 mol) in glacial acetic acid (500 mL) cooled to 5° C. was added dropwise a cold solution of sodium nitrite (32.5 g, 0.446 mol) in water (50 mL). The mixture was stirred for 1 h and allowed to stand for 4 h, during which time it warmed to room temperature. The mixture was used in the next step without further purification. LCMS: m/z 188 (M+1).

### Step 1c. 2-tert-Butyl 4-ethyl 3,5-dimethyl-1H-pyrrole-2,4-dicarboxylate (Compound 105)

The above mixture (104) was stirred and portions of zinc powder (84 g, 1.29 mol) were added at such a rate that the mixture temperature was blow 80° C. After the addition was completed, the mixture was heated to 60° C. for 1 h. Ethyl acetylacetate (60 g, 0.46 mol) was added into above mixture and the mixture was refluxed at 85° C. for 4 h. The mixture was filtered to remove the zinc powder when it was hot, the filtrate was poured into 1 L of ice and stand overnight. The precipitate was filtered to obtain product 105 (29 g, 24.7%), the solid was used in next step without further purification. LCMS: m/z 268 (M+1).

### Step 1d. Ethyl 2,4-dimethyl-1H-pyrrole-3-carboxylate (Compound 106)

A solution of 105 (12 g, 45 mmol) in ethanol (325 mL) was treated with 1 M H₂SO₄ (240 mL). The solution was stirred at 65° C. for 4 h, and then cooled to room temperature and evaporated most of ethanol, extracted with dichloromethane. The organic layer was combined and dried with MgSO₄. After removal of the solvent, the crude product 506 (3 g, 40%) was obtained. The crude product was purified by column chromatography (silica gel, elution 10/1 petroleum/ethyl acetate) to obtain brown solid product 106 (1.5 g, 20%). LCMS: m/z 168 (M+1), ¹H NMR (DMSO-d₆) δ2.10 (s, 3H), 2.35 (s, 3H), 4.13 (q, 2H), 6.37 (s, 1H), 10.85 (s, 1H).

### Step 1e. Ethyl 5-formyl-2,4-dimethyl-1H-pyrrole-3-carboxylate (Compound 107)

To a solution of DMF (2 g, 27 mmol) at 10° C. was added POCl₃ (2.6 mL) in 10 mL of dichloromethane through the dropping funnel over a period of 30 min. After addition, the mixture was stirred for 20 min at room temperature. Dichloromethane (10 mL) was added into the mixture. When the internal temperature lowed to 5° C., a solution of compound 506 in dichloromethane (10 mL) was added through a dropping funnel to the stirred, cooled mixture over a period of 1 h, then the mixture was stirred at the reflux temperature for 30 min, the mixture was then cooled to 30° C., a solution of sodium acetate (17 g, 125 mmol) in water (100 ml) was added. The reaction mixture was again refluxed for 30 min. then cooled to room temperature, the aqueous layer was extracted with dichloromethane (4×100 mL). The combined organic layer were washed with brine, dried and evaporated to give gray solid product 107 (4.42 g, 90%). LCMS: m/z 196 (M+1), ¹H NMR (DMSO-d₆) δ1.35 (t, J3H), 2.23 (s, 3H), 2.48 (s, 3H), 4.12 (q, 2H), 9.60 (s, 1H), 10.58 (s, 1H).

### Step 1f. 5-Formyl-2,4-dimethyl-1H-pyrrole-3-carboxylic acid (compound 108)

A solution of KOH (6.2 g, 111 mmol) in water (400 mL) was added to a solution of compound 507 (7.2 g, 37 mmol) in ethanol (60 mL). The mixture was refluxed until the reaction was completed. The mixture was cooled to room temperature and the aqueous layer was extracted with dichloromethane. The aqueous layer was acidified to pH=2 with 1N HCl. The precipitate was collected by filtration, washed with water and dried to give yellow solid product 108 (5.5 g, 89%). LCMS: m/z 168 (M+1), ¹H NMR (DMSO-d₆) δ2.40 (s, 3H), 2.43 (s, 3H), 9.24 (s, 1H), 12.14 (bs, 2H).

### Step 1g. 5-((Z)-(5-fluoro-2-oxoindolin-3-ylidene)methyl)-N-hydroxy-2,4-dimethyl-1H-pyrrole-3-carboxamide (compound 1)

A mixture of compound 108 (4.0 g. 24 mmol), 102 (3.6 g 24 mmol) and pyrrolidine (2 mL) in ethanol (200 mL) was stirred and heated at 78° C. for 6 h. The mixture was filtered to give yellow solid, dried to yield product 1 (5.5 g, 77%). LCMS: m/z 301 (M+1), ¹H NMR (DMSO-d₆) δ2.39 (s, 3H), 2.42 (s, 3H), 6.82 (m, 2H), 7.77 (s, 1H), 7.80 (m, 1H), 10.93 (s, 1H), 12.23 (s, 1H), 13.86 (s, 1H).

### Example 2

### Preparation of N-(2-(hydroxycarbamoyl)ethyl)-5-((Z)-(5-fluoro-2-oxoindolin-3-ylidene)methyl)-2,4-dimethyl-1H-pyrrole-3-carboxamide (compound 2)

### Step 2a. Methyl 3-(5-((Z)-(5-fluoro-2-oxoindolin-3-ylidene)methyl)-2,4-dimethyl-1H-pyrrole-3-carboxamido)propanoate (compound 110-2)

To a stirred solution of 1 (0.5 g, 1.67 mmol) in DMF (35 mL) at room temperature was added HOBt (1.02 g, 7.52 mmol), triethylamine (2.12 mL, 15.03 mmol), ECDI.HCl (1.44 g, 1.52 mmol) and methyl 3-aminopropanate hydrochloride (0.7 g, 5.0 mmol) successively. The mixture was stirred for 24 h at room temperature and then was diluted with water (20 mL), brine (20 mL) and saturated bicarbonate solution (20 mL) and the pH of solution was adjusted to 11˜12 with 10 mol/L NaOH. The mixture was filtrated and the solid was collected washed with water, dried to obtain crude yellow solid product 110-2 (0.44 g, 68.3%). LCMS: m/z 386 (M+1), ¹H NMR (DMSO-d₆) δ2.38 (s, 3H), 2.41 (s, 3H), 2.50 (t, 2H), 3.44 (t, 2H), 3.62 (s, 3H), 6.85 (m, 2H), 7.71 (m, 3H), 10.86 (s, 1H), 13.69 (s, 1H).

### Step 2b. N-(2-(Hydroxycarbamoyl)ethyl)5-((Z)-(5-fluoro-2-oxoindolin-3-ylidene)methyl)-2,4-dimethyl-1H-pyrrole-3-carboxamide (compound 2)

NaH (60%, 936 mg, 23.4 mmol g) was added to the solution of hydroxy-ammonium chloride (1.08 g, 15.6 mmol) in DMF (25 mL) drop portion at ice bath. After 0.5 h, the solution of 110-2 (0.2 g, 0.52 mmol) in DMSO (3 mL) was added to the above mixture. The mixture was stirred for 2 h at 0° C., filtration, the residue was washed with DMF, and the DMF was removed under reduce pressure, purified to obtain yellow solid 2 (25 mg, 12.5%). LCMS: m/z 387 (M+1), ¹H NMR (DMSO-d₆) δ2.25 (t, 2H) 2.41 (s, 3H), 2.43 (s, 3H), 6.85 (m, 2H), 7.64 (t, 1H), 7.71 (s, 1H), 7.727 (m, 1H), 8.75 (s, 1H), 10.47 (s, 1H), 10.89 (s, 1H), 13.68 (s, 1H).

### Example 3

### Preparation of N-(3-(hydroxycarbamoyl)propyl)5-((Z)-(5-fluoro-2-oxoindolin-3-ylidene)methyl)-2,4-dimethyl-1H-pyrrole-3-carboxamide (compound 3)

### Step 3a. Methyl 4-(5-((Z)-(5-fluoro-2-oxoindolin-3-ylidene)methyl)-2,4-dimethyl-1H-pyrrole-3-carboxamido)butanoate (Compound 110-3)

To a stirred solution of compound 1 (0.5 g, 1.67 mmol) in DMF (35 mL) at room temperature was added HOBt (1.02 g, 7.52 mmol), triethylamine (2.12 mL, 15.03 mmol), ECDI.HCl (1.44 g, 1.52 mmol) and methyl 4-aminobutanate hydrochloride (0.77 g, 5.0 mmol) successively. The mixture was stirred for 24 h at room temperature and then was diluted with water (20 mL), brine (20 mL) and saturated bicarbonate solution (20 mL) and the pH of solution was adjusted to 11˜12 with 10 mol/L NaOH. The mixture was filtrated, the solid was collected, washed with water and dried to obtain crude yellow solid product 110-3 (0.32 g, 48.3%). LCMS: m/z 400 (M+1), ¹H NMR (DMSO-d₆) δ1.77 (m, 2H), 2.39 (m, 4H), 2.42 (s, 3H), 2.49 (s, 3H), 3.23 (t, 2H), 6.85 (m, 2H), 7.60 (t, 1H), 7.67 (s, 1H), 7.71 (m, 1H), 10.89 (s, 1H), 13.68 (s, 1H).

### Step 3b. N-(3-(Hydroxycarbamoyl)propyl)5-((Z)-(5-fluoro-2-oxoindolin-3-ylidene)methyl)-2,4-dimethyl-1H-pyrrole-3-carboxamide (compound 3)

NaH (60%, 900 mg, 22.5 mmol g) was added to a solution of hydroxyamine hydrochloride (1.04 g, 15 mmol) in DMF (25 mL) portionwise at ice bath. After 0.5 h, the solution of compound 110-3 (0.2 g, 0.5 mmol) in DMSO (3 mL) was added to the above mixture. The mixture was stirred for 2 h at 0° C. The reaction was filtered and the residue was washed with DMF, dried to remove remaining DMF to yield yellow solid product 3 (21.0 mg, 10.5%). LCMS: m/Z 401 (M+1), ¹H NMR (DMSO-d₆) δ1.73 (t, 2H), 2.02 (t, 2H), 2.38 (s, 3H), 2.41 (s, 3H), 2.43 (t, 2H), 6.85 (m, 2H), 7.66 (t, 1H), 7.72 (s, 1H), 7.76 (m, 1H), 8.70 (s, 1H), 10.40 (s, 1H), 10.88 (s, 1H), 13.68 (s, 1H).

### Example 4

### Preparation of N-(5-(hydroxycarbamoyl)pentyl)5-((Z)-(5-fluoro-2-oxoindolin-3-ylidene)methyl)-2,4-dimethyl-1H-pyrrole-3-carboxamide (compound 4)

### Step 4a. Methyl 6-(5-((Z)-(5-fluoro-2-oxoindolin-3-ylidene)methyl)-2,4-dimethyl-1H-pyrrole-3-carboxamido)hexanoate (compound 110-4)

To a stirred solution of compound 1 (0.5 g, 1.67 mmol) in DMF (35 mL) at room temperature was added HOBt (1.02 g, 7.52 mmol), triethylamine (2.12 mL, 15.03 mmol), ECDI.HCl (1.44 g, 1.52 mmol) and methyl 6-aminohexanate hydrochloride (0.91 g, 5.0 mmol) successively. The mixture was stirred for 24 h at room temperature and then was diluted with water (20 mL), brine (20 mL) and saturated bicarbonate solution (20 mL) and the pH of solution was adjusted to 11˜12 with 10 M NaOH. The mixture was filtrated and the resulting solid was washed with water and dried to obtain crude yellow solid product 110-4 (0.47 g, 65.8%). LCMS: m/z 428 (M+1), ¹H NMR (DMSO-d₆) δ1.33 (m, 2H), 1.54 (m, 4H), 2.32 (t, 2H), 2.42 (s, 3H), 2.50 (s, 3H), 3.20 (t, 2H), 3.59 (s, 3H), 6.85 (m, 2H), 7.60 (t, 1H), 7.69 (s, 1H), 7.71 (m, 1H), 10.88 (s, 1H), 13.67 (s, 1H).

### Step 4b. N-(5-(Hydroxycarbamoyl)pentyl)5-((Z)-(5-fluoro-2-oxoindolin-3-ylidene)methyl)-2,4-dimethyl-1H-pyrrole-3-carboxamide (compound 4)

NaH (60%, 846 mg, 21.3 mmol g) was added to the solution of hydroxyamine hydrochloride (0.93 g, 14.0 mmol) in DMF (25 mL) portionwise at 0° C. After 0.5 h, the solution of 110-4 (0.2 g, 0.47 mmol) in DMSO (3 mL) was added to the above mixture. The mixture was stirred for 2 h at 0° C. and filtered. The collected solid was washed with DMF, and the remaining DMF was removed under reduce pressure to obtain yellow solid product 4 (22.6 mg, 11.2%). m.p. 209.7° C. (decompose), LCMS: m/z 429 (M+1), ¹H NMR (DMSO-d₆) δ1.27 (m, 2H), 1.48 (m, 4H), 1.94 (t, 2H), 2.38 (s, 3H), 2.40 (s, 3H), 3.12 (t, 2H), 6.87 (m, 2H), 7.60 (t, 1H), 7.69 (s, 1H), 7.72 (m, 1H), 8.63 (s, 1H), 10.32 (s, 1H), 10.82 (s, 1H), 13.65 (s, 1H).

### Example 5

### Preparation of (Z)-5-((5-fluoro-2-oxoindolin-3-ylidene)methyl)-N-(2-(4-(hydroxycarbamoyl)phenoxy)ethyl)-2,4-dimethyl-1H-pyrrole-3-carboxamide (Compound 8)

### Step 5a. Methyl 4-(cyanomethoxy)benzoate (Compound 201)

To a solution of methyl 4-hydroxylbenzoate (5.0 g, 32.9 mmol) in DMF (50 mL) was added 2-Chloroacetonitrile (2.5 g, 32.9 mmol) and K₂CO₃ (13.6 g, 98.6 mmol). The mixture was stirred at 50° C. for 4 h. Water (100 ml) was added and resulting solid was filtered to give product 201 as a white solid (6.2 g, 98%). The solid was used in the next step without further purification. LCMS: 192 [M+1]⁺.

### Step 5b. Methyl 4-(2-aminoethoxy)benzoate (Compound 202)

A solution of compound 201 (1.5 g, 7.8 mmol) in THF (15 mL) was stirred at refluxing temperature under N₂ atmosphere. BH₃ Me₂S (3.9 mL, 7.8 mmol) was added dropwise over 30 minutes. The solution was refluxed for 4 hours and then cooled to room temperature. 6N HCl (3 ml) was added and the mixture was refluxed for 0.5 hours and then cooled to 0° C. The reaction mixture was filtered and the filtrate was concentrated to give crude product 202 as a white solid (2.3 g). The crude product was used in the next step without further purification. LCMS: 196 [M+1]⁺.

### Step 5c. (Z)-Methyl 4-(2-(5-((5-fluoro-2-oxoindolin-3-ylidene)methyl)-2,4-dimethyl-1H-pyrrole-3-carboxamido)ethoxy)benzoate (Compound 203)

To a stirred solution of compound 109 (0.5 g, 1.67 mmol) in THF (150 mL) at 0° C. was added HOBt (0.34 g, 2.8 mmol), triethylamine (0.6 mL, 4.18 mmol), ECDI.HCl (0.48 g, 2.8 mmol) and compound 202 (0.6 g, 3.33 mmol) successively. The mixture was stirred overnight at room temperature, evaporated to remove solvent, diluted with water (50 mL), brine (50 mL) and saturated sodium bicarbonate solution (50 mL). The pH of solution was adjusted to 11˜12 with 10M NaOH. The mixture was filtered, washed with water, dried to obtain crude yellow solid 203 (550 mg, 69%). LCMS: 478 [M+1], ¹H NMR (DMSO-d₆): δ 2.40 (s, 3H), 2.43 (s, 3H), 3.63 (m, 2H), 3.82 (s, 3H), 4.20 (t, 2H), 6.92 (m, 2H), 7.07 (d, 2H), 7.70 (m, 2H), 7.84 (s, 1H), 7.91 (d, 2H), 10.88 (s, 1H), 13.68 (s, 1H).

### Step 5d. (Z)-4-(2-(5-((5-Fluoro-2-oxoindolin-3-ylidene)methyl)-2,4-dimethyl-1H-pyrrole-3-carboxamido)ethoxy)benzoic acid (Compound 8)

NaH (60%, 650 mg, 15.75 mmol) was added to the solution of hydroxylamine hydrochloride (750 mg, 10.5 mmol) in DMF (15 mL) portionwise at 0° C. After 0.5 h, the solution of compound 203 (500 mg, 1.05 mmol) in DMF (25 mL) was added to the above mixture. The mixture was stirred for 0.5 h at 0° C. and filtered. The solid was washed with DMF, and the filtrate was concentrated under reduced pressure to obtain a yellow solid that was purified to give product 8 as a yellow solid (65 mg, 17%). LCMS: 479 [M+1]⁺, ¹H NMR (DMSO-d₆): δ 2.39 (s, 3H), 2.41 (s, 3H), 3.59 (m, 2H), 4.15 (t, J=5.7 Hz, 2H), 6.83 (m, 4H), 7.69 (m, 5H), 8.85 (s, 1H), 10.84 (s, 1H), 11.02 (s, 1H), 13.67 (s, 1H).

### Example 6

### Preparation of (Z)-5-((5-fluoro-2-oxoindolin-3-ylidene)methyl)-N-(6-(hydroxy(methyl)amino)-6-oxohexyl)-2,4-dimethyl-1H-pyrrole-3-carboxamide (Compound 9)

NaH (60%, 700 mg, 17.55 mmol) was added to a solution of N-methyl hydroxylamine hydrochloride (1 g, 11.7 mmol) in DMF (15 mL) portionwise at 0° C. After 0.5 h, a solution of compound 110-4 (0.5 g, 1.15 mmol) in DMF (25 mL) was added. The mixture was stirred for 15 min at 0° C., filtered and washed with DMF. The filtrate was concentrated under reduced pressure to obtain crude yellow solid that was purified to give desired product 9 as a yellow solid (150 mg, 35%). LCMS: 443 [M+1]⁺, ¹H NMR (DMSO-d₆): δ 1.28 (m, 2H), 1.47 (m, 4H), 2.31 (m, 2H), 2.38 (s, 3H), 2.40 (s, 3H), 3.06 (s, 3H), 3.15 (m, 2H), 6.83 (m, 2H), 7.60 (t, J=5.85 Hz, 1H), 7.69 (s, 1H), 7.73 (m, 1H), 9.72 (s, 1H), 10.86 (s, 1H), 13.65 (s, 1H).

### Example 7

### Preparation of N-(2-aminophenyl)-5-((Z)-(5-fluoro-2-oxoindolin-3-ylidene)methyl)-2,4-dimethyl-1H-pyrrole-3-carboxamide (Compound 10)

To a stirred solution of compound 109 (0.2 g, 0.67 mmol) in DMF (30 mL) at 0° C. was added HOBt (0.136 g, 1.0 mmol), triethylamine (0.24 mL, 1.67 mmol), ECDI.HCl (0.192 g, 1.0 mmol) and benzene-1,2-diamine (0.2 g, 2.0 mmol) successively. The mixture was stirred for 72 h at room temperature, diluted with water (20 mL), brine (20 mL) and saturated aqueous sodium bicarbonate (20 mL). The pH of solution was adjusted to 11˜12 with 10M NaOH. The mixture was filtered, washed with water, dried to obtain the product 10 as a yellow solid (0.13 g, 50.03%). LCMS: 391 [M+1], ¹H NMR (DMSO-d₆): δ 4.83 (s, 2H), 6.58 (t, J=7.2 Hz, 1H), 6.78 (d, 1H), 6.84 (m, 1H), 6.92 (t, J=7.8 Hz, 2H), 7.25 (d, 1H), 7.74 (m, 2H), 9.00 (s, 1H), 10.90 (d, 1H), 13.75 (s, 1H), ¹H NMR (DMSO-D₂O) δ 2.41 (s, 3H), 2.44 (s, 3H), 6.62 (t, J=7.4 Hz, 1H), 6.78 (d, 1H), 6.89 (m, 1H), 6.95 (m, 2H), 7.19 (d, 1H), 7.67 (m, 2H).

### Example 8

### Preparation of 5-((Z)-(5-fluoro-2-oxoindolin-3-ylidene)methyl)-N-(2-(4-((E)-3-(hydroxyamino)-3-oxoprop-1-enyl)phenoxy)ethyl)-2,4-dimethyl-1H-pyrrole-3-carboxamide (compound 14)

### Step 8a. (E)-Methyl 3-(4-(2-bromoethoxy)phenyl)acrylate (Compound 301)

To a solution of (E)-methyl 3-(4-hydroxylphenyl)acrylate (2.0 g, 11.24 mmol) in DMF (2.5 mL) was added 1,2-Dibromoethane (40 ml), K₂CO₃ (4.66 g, 33.7 mmol). The mixture was stirred at 90° C. for 6 hour and filtered. The filtrate was evaporated to give product 301 as a white solid (3.05 g, 95.2%). LCMS: 286 [M+1]⁺.

### Step 8b. (E)-Methyl 3-(4-(2-(1,3-dioxoisoindolin-2-yl)ethoxy)phenyl)acrylate (Compound 302)

A mixture of compound 301 (1.5 g, 5.26 mmol), potassium phthalimide (1.07 g, 5.79 mmol) in DMF (20 mL) was stirred for 4 hours at 100° C. The reaction was cooled and the resulting solid was filtered. The filtrate was concentrated under reduced pressure to give product 302 as a white solid (1.75 g, 95.1%). LCMS: 352 [M+1]⁺.

### Step 8c. (E)-Methyl 3-(4-(2-aminoethoxy)phenyl)acrylate (Compound 303)

To a suspension of compound 302 (1.85 g, 5.26 mmol) in EtOH (25 ml) was added hydrazine hydrate (0.4 mL, 7.89 mmol). The resulting mixture was refluxed for 10 hours and filtered. The filtrate concentrated to give desired product 303 (1.1 g, 95%). LCMS: 222 [M+1]⁺.

### Step 8d. (E)-Methyl 3-(4-(2-(5-((Z)-(5-fluoro-2-oxoindolin-3-ylidene)methyl)-2,4-dimethyl-1H-pyrrole-3-carboxamido)ethoxy)phenyl)acrylate (Compound 304)

To a stirred solution of compound 109 (0.5 g, 1.67 mmol) in DMF (40 mL) at 0° C. was added HOBt (0.34 g, 2.5 mmol), triethylamine (0.94 mL, 6.68 mmol), ECDI.HCl (0.48 g, 2.5 mmol) and compound 303 (0.44 g, 2.0 mmol) successively. The mixture was stirred overnight at room temperature, evaporated, diluted with water (50 mL), brine (50 mL) and saturated aqueous sodium bicarbonate (50 mL). The pH of solution was adjusted to 11˜12 with 10M NaOH. The mixture was filtered, washed with water, dried to obtain desired product 304 as a yellow solid (630 mg, 75%). LCMS: 504 [M+1]⁻, ¹H NMR (DMSO-d₆): δ 2.39 (s, 3H), 2.41 (s, 3H), 3.59 (m, 2H), 3.69 (s, 3H), 4.15 (t, J=4.5 Hz, 2H), 6.45 (d, 1H), 6.94 (m, 4H), 7.65 (m, 6H), 10.87 (s, 1H), 13.66 (s, 1H).

### Step 8e. 5-((Z)-(5-Fluoro-2-oxoindolin-3-ylidene)methyl)-N-(2-(4-((E)-3-(hydroxyamino)-3-oxoprop-1-enyl)phenoxy)ethyl)-2,4-dimethyl-1H-pyrrole-3-carboxamide (Compound 14)

NaH (60%, 894 mg, 22.3 mmol) was added to the solution of hydroxylamine hydrochloride (1.035 g, 14.9 mmol) in DMF (15 mL) portionwise at 0° C. After 0.5 h, the solution of compound 304 (750 mg, 1.49 mmol) in DMSO (40 mL) was added to the above mixture. The mixture was stirred for 15 minutes at 0° C. and filtered, washed with DMF, and the filtrate was concentrated under reduced pressure. The residue was purified to give title compound 14 as a yellow solid (25 mg, 3.3%). LCMS: 505 [M+1]⁺, ¹H NMR (DMSO-d₆): δ 2.38 (s, 3H), 2.41 (s, 3H), 3.58 (m, 2H), 4.13 (t, J=5.4 Hz, 2H), 6.27 (d, 1H), 6.98 (m, 4H), 7.41 (d, 1H), 7.48 (d, 2H), 7.69 (s, 1H), 7.75 (m, 1H), 7.81 (m, 1H), 10.87 (s, 1H), 13.66 (s, 1H).

### Example 9

### Preparation of (Z)-5-((5-fluoro-2-oxoindolin-3-ylidene)methyl)-N-(7-(hydroxyl-amino)-7-oxoheptyl)-2,4-dimethyl-1H-pyrrole-3-carboxamide (Compound 15)

### Step 9a. (Z)-Methyl 7-(5-((5-fluoro-2-oxoindolin-3-ylidene)methyl)-2,4-dimethyl-1H-pyrrole-3-carboxamido)heptanoate (Compound 110-15)

To a stirred solution of compound 109 (220.0 mg, 0.73 mmol) in DMF (15 mL) at room temperature was added HOBt (148.6 mg, 1.1 mmol), triethylamine (0.21 mL, 1.46 mmol), ECDI.HCl (210.2 mg, 1.1 mmol) and methyl 7-aminoheptanoate hydrochloride (157.1 mg, 0.8 mmol) successively. The mixture was stirred for 24 h at room temperature and was then diluted with water (20 mL), brine (20 mL) and saturated bicarbonate solution (20 mL). The pH of the mixture was adjusted to 11˜12 with 10 N NaOH. The mixture was filtered and the solid was washed with water, dried to obtain a crude product 110-15 as a yellow solid (0.3 g, 93.2%). LCMS: 442 [M+1]⁺; ¹H NMR (DMSO-d₆): δ 1.31 (m, 4H), 1.50 (m, 4H), 2.31 (t, J=7.35 Hz, 2H), 2.40 (s, 3H), 2.42 (s, 3H), 3.19 (m, 2H), 3.59 (s, 3H), 6.87 (m, 2H), 7.71 (m, 3H), 10.91 (s, 1H), 13.67 (s, 1H).

### Step 9b. (Z)-5-((5-Fluoro-2-oxoindolin-3-ylidene)methyl)-N-(7-(hydroxyamino)-7-oxoheptyl)-2,4-dimethyl-1H-pyrrole-3-carboxamide (Compound 15)

The NaH (60%, 140 mg, 3.5 mmol g) was added to the solution of hydroxylamine hydrochloride (160 mg, 2.3 mmol) in DMF (3 mL) at ice bath temperature and stirred for 0.5 h. To the mixture was added the solution of 110-15 (100.0 mg, 0.23 mmol) in DMSO (5 mL). The resulting mixture was stirred for 0.5 h at 0° C. and filtered. The solid was washed with DMF. The combined filtrate was concentrated under reduced pressure to give a residue which was purified by preparative HPLC to afford the title compound as a yellow solid (63 mg, 63%). m.p. 221° C. (decomp.) LCMS: 443 [M+1]; ¹H NMR (DMSO-d₆): δ 1.29 (m, 4H) 1.48 (m, 4H), 1.93 (t, J=7.2 Hz, 2H), 2.38 (s, 3H), 2.40 (s, 3H), 3.19 (m, 2H), 6.87 (m, 2H), 7.69 (m, 3H), 10.31 (s, 1H), 10.87 (s, 1H), 13.65 (s, 1H).

### Example 10

### Preparation of (Z)-5-((5-fluoro-2-oxoindolin-3-ylidene)methyl)-N-(8-(hydroxylamino)-8-oxooctyl)-2,4-dimethyl-1H-pyrrole-3-Carboxamide (Compound 16)

### Step 10a. (Z)-Methyl 8-(5-((5-fluoro-2-oxoindolin-3-ylidene)methyl)-2,4-dimethyl-1H-pyrrole-3-carboxamido)octanoate (Compound 110-16)

To a stirred solution of compound 109 (500 mg, 1.67 mmol) in DMF (40 mL) at room temperature was added HOBt (337.8 mg, 2.5 mmol), triethylamine (0.94 mL, 6.68 mmol), ECDI.HCl (477.8 mg, 2.5 mmol) and methyl 8-aminooctanoate hydrochloride (385.3 mg, 1.84 mmol) successively. The mixture was stirred for 24 h at room temperature and diluted with water (20 mL), brine (20 mL) and saturated sodium bicarbonate solution (20 mL). The pH of solution was adjusted to 11˜12 with 10 N NaOH. The mixture was filtered and the solid was washed with water, dried to obtain a crude product 110-16 as a yellow solid (0.62 g, 86.1%). LCMS: 456 [M+1]; ¹H NMR (DMSO-d₆): δ 1.28 (m, 6H), 1.50 (m, 4H), 2.28 (t, J=7.35 Hz, 2H), 2.38 (s, 3H), 2.40 (s, 3H), 3.20 (m, 2H), 3.56 (s, 3H), 6.84 (m, 2H), 7.69 (m, 3H), 10.87 (s, 1H), 13.65 (s, 1H).

### Step 10b. (Z)-5-((5-Fluoro-2-oxoindolin-3-ylidene)methyl)-N-(8-(hydroxyamino)-8-oxooctyl)-2,4-dimethyl-1H-pyrrole-3-carboxamide (Compound 16)

The NaH (60%, 736 mg, 18.4 mmol) was added to the solution of hydroxylamine hydrochloride (855 mg, 12.3 mmol) in DMF (15 mL) at ice bath temperature and stirred for 0.5 h. To the mixture was added the solution of compound 110-16 (560 mg, 1.23 mmol) in DMSO (25 mL). The resulting mixture was stirred for 0.5 hours at 0° C. and filtered. The solid was washed with DMF. The combined filtrate was concentrated under reduced pressure to give a residue which was purified by preparative HPLC to afford product 16 as a yellow solid (40 mg, 7%). m.p. 213.7° C. (decomp.). LCMS: 457 [M+1]⁺; ¹H NMR (DMSO-d₆): δ 1.27 (m, 6H) 1.47 (m, 4H), 1.92 (t, J=6.9 Hz, 2H), 2.38 (s, 3H), 2.40 (s, 3H), 3.18 (m, 2H), 6.87 (m, 2H), 7.70 (m, 3H), 8.66 (s, 1H), 10.32 (s, 1H), 10.88 (s, 1H), 13.66 (s, 1H).

### Example 11

### Preparation of (Z)-N-(6-(acetoxyamino)-6-oxohexyl)-5-((5-fluoro-2-oxoindolin-3-ylidene)methyl)-2,4-dimethyl-1H-pyrrole-3-carboxamide (Compound 17)

Ac₂O (1.5 ml) was added to a solution of compound 4 (120 mg, 0.28 mmol) in AcOH (15 mL). The solution was stirred at room temperature for 4 hours. The mixture was adjusted to pH 7˜8 with saturated aqueous NaHCO₃. The resulting solid was collected by filtration. The residue was washed with water three times, dried to give desired product 17 as a yellow solid (100 mg, 76%). LCMS: 471 [M+1], ¹H NMR (DMSO-d₆): δ 1.32 (m, 2H), 1.53 (m, 4H), 2.12 (m, 5H), 2.39 (s, 3H), 2.41 (s, 3H), 3.20 (m, 2H), 6.85 (m, 2H), 7.581 (m, 1H), 7.69 (m, 2H), 10.84 (s, 1H), 11.52 (s, 1H), 13.65 (s, 1H).

### Example 12

### Preparation of (Z)-5-((5-fluoro-2-oxoindolin-3-ylidene)methyl)-N-(6-(isobutyryloxyamino)-6-oxohexyl)-2,4-dimethyl-1H-pyrrole-3-carboxamide (compound 18)

Isobutyric anhydride (7 mL, 42.2 mmol) was added to a solution of compound 4 (500 mg, 1.17 mmol) in AcOH (70 mL). The solution was stirred at room temperature for 4 hours and the mixture was adjusted to pH 7˜8 with saturated aqueous NaHCO₃. The resulting solid was collected by filtration, washed with water for three times, dried and purified by preparative HPLC to give product 18 as a yellow solid (35 mg, 6%). LCMS: 499 [M+1]⁺, ¹H NMR (DMSO-d₆): δ 1.12 (s, 3H), 1.14 (s, 3H), 1.34 (m, 2H), 1.55 (m, 4H), 2.11 (t, J=6.9 Hz, 2H), 2.39 (s, 3H), 2.41 (s, 3H), 2.69 (m, 1H), 3.18 (m, 2H), 6.83 (m, 2H), 7.63 (m, 3H), 10.88 (s, 1H), 11.54 (s, 1H), 13.66 (s, 1H).

### Example 13

### Preparation of (Z)-N-(6-(benzoyloxyamino)-6-oxohexyl)-5-((5-fluoro-2-oxoindolin-3-ylidene)methyl)-2,4-dimethyl-1H-pyrrole-3-carboxamide (Compound 19)

Benzoic anhydride (200 mg, 0.88 mmol) was added to a solution of compound 4 (200 mg, 0.47 mmol) in AcOH (40 mL). The solution was stirred at room temperature for 4 hours and then adjusted to pH 7˜8 with saturated aqueous NaHCO₃. The resulting solid was collected by filtration, washed with water for three times, dried and purified by preparative HPLC to give product 19 as a yellow solid (40 mg, 16%). LCMS: 533 [M+1]⁻, ¹H NMR (DMSO-d₆): δ 1.35 (m, 2H), 1.53 (m, 4H), 2.12 (t, J=7.05 Hz, 2H), 2.40 (s, 3H), 2.41 (s, 3H), 3.20 (m, 2H), 6.85 (m, 2H), 7.57 (m, 1H), 7.70 (m, 3H), 7.99 (s, 1H), 8.01 (s, 1H), 10.88 (s, 1H), 11.88 (s, 1H), 13.66 (s, 1H).

### Example 14

### Preparation of (Z)-5-((5-fluoro-2-oxoindolin-3-ylidene)methyl)-2,4-dimethyl-N-(6-oxo-6-(propionyloxyamino)hexyl)-1H-pyrrole-3-carboxamide (Compound 20)

Propionic anhydride (7 mL, 54.4 mmol) was added to a solution of compound 4 (500 mg, 1.17 mmol) in AcOH (70 mL). The solution was stirred at room temperature for 4 hours and the mixture was adjusted to pH 78 with saturated NaHCO₃. The mixture was filtered, washed with water for three times, dried and purified by preparative HPLC to give product 20 as a yellow solid (180 mg, 32%). LCMS: 485 [M+1], ¹H NMR (DMSO-d₆): δ 1.06 (t, J=7.8 Hz, 3H), 1.31 (m, 2H), 1.52 (m, 4H), 2.11 (t, J=7.35 Hz, 2H), 2.38 (s, 3H), 2.40 (s, 3H), 2.44 (m, 2H), 3.17 (m, 2H), 6.82 (m, 2H), 7.66 (m, 3H), 10.84 (s, 1H), 11.51 (s, 1H), 13.64 (s, 1H).

### Example 15

### Preparation of (Z)-N-(6-(cyclohexanecarbonyloxyamino)-6-oxohexyl)-5-((5-fluoro-2-oxoindolin-3-ylidene)methyl)-2,4-dimethyl-1H-pyrrole-3-carboxamide (Compound 21)

Cyclohexanecarboxylic anhydride (5 mL) and cyclohexanecarboxylic acid (150 mg, 1.17 mmol) was added to a solution of compound 4 (500 mg, 1.17 mmol) in THF (120 ml) and DMF (5 mL). The solution was stirred at room temperature for 4 h. THF was removed in vacuo, and then water (100 mL) was added. The mixture was adjusted to pH 78 with saturated aqueous NaHCO₃. The resulting solid was collected by filtration, washed with water for three times, dried and purified by preparative HPLC to give product 21 as a yellow solid (100 mg, 13%). LCMS: 539 [M+1]⁺, ¹H NMR (DMSO-d₆): δ 1.32 (m, 7H), 1.53 (m, 5H), 1.66 (m, 2H), 1.85 (m, 2H), 2.11 (t, J=6.45 Hz, 2H), 2.39 (s, 3H), 2.41 (s, 3H), 3.20 (m, 2H), 6.85 (m, 2H), 7.71 (m, 3H), 10.89 (s, 1H), 11.54 (s, 1H), 13.67 (s, 1H).

### Example 16

### Preparation of (Z)-N-(7-(acetoxyamino)-7-oxoheptyl)-5-((5-fluoro-2-oxoindolin-3-ylidene)methyl)-2,4-dimethyl-1H-pyrrole-3-carboxamide (Compound 28)

Ac₂O (2 ml) was added to a solution of compound 15 (140 mg, 0.32 mmol) in 20 ml AcOH. The solution was stirred at room temperature for 4 h. Saturated NaHCO₃ was added slowly to adjust PH to 78. The solid was collected by filtration, washed with water for three times, dried to give crude product which was purified by prep-HPLC to give product 28 (95 mg, 62%). LCMS: 485 [M+1]⁺, ¹H NMR (DMSO-d₆) δ 1.30 (m, 4H), 1.51 (m, 4H), 2.07 (m, 5H), 2.38 (s, 3H), 2.40 (s, 3H), 3.19 (m, 2H), 6.84 (m, 2H), 7.62 (t, J=6.0 Hz, 1H), 7.69 (s, 1H), 7.75 (m, 1H), 10.87 (s, 1H), 11.54 (s, 1H), 13.65 (s, 1H).

### Example 17

### Preparation of ethyl (Z)-N-(8-(acetoxyamino)-8-oxooctyl)-5-((5-fluoro-2-oxoindolin-3-ylidene)methyl)-2,4-dimethyl-1H-pyrrole-3-carboxamide (Compound 29)

Ac₂O (3 ml) was added to a solution of compound 16 (228 mg, 0.5 mmol) in 30 ml AcOH. The solution was stirred at room temperature for 4 h. Saturated NaHCO₃ was added slowly to adjust PH to 7˜8. The solid was collected by filtration, washed with water for three times, dried to give crude product which was purified by prep-HPLC to give product 29 (50 mg, 20%). LCMS: 499 [M+1]⁺, ¹H NMR (DMSO-d₆) δ 1.29 (m, 6H), 1.49 (m, 4H), 2.07 (t, 2H), 2.12 (s, 3H), 2.38 (s, 3H), 2.40 (s, 3H), 3.20 (m, 2H), 6.85 (m, 2H), 7.63 (t, J=5.6 Hz, 1H), 7.70 (s, 1H), 7.76 (m, 1H), 10.88 (s, 1H), 11.53 (s, 1H), 13.65 (s, 1H).

### Biological Assays:

As stated hereinbefore the derivatives defined in the present invention possess anti-proliferation activity. These properties may be assessed, for example, using one or more of the procedures set out below:

(a) An In Vitro Assay which Determines the Ability of a Test Compound to Inhibit a Receptor Tyrosine Kinase.

The ability of compounds to inhibit receptor kinase (VEGFR2 and PDGFR-beta) activity was assayed using HTScan™ Receptor Kinase Assay Kits (Cell Signaling Technologies, Danvers, Mass.). VEGFR2 tyrosine kinase was produced using a baculovirus expression system from a construct containing a human VEGFR2 cDNA kinase domain (Asp805-Val1356) (GenBank accession No. AF035121) fragment amino-terminally fused to a GST-HIS6-Thrombin cleavage site. PDGFR-beta tyrosine kinase was produced using a baculovirus expression system from a construct containing a human PDGFR-beta c-DNA (GenBank Accession No. NM_{—}002609) fragment (Arg561-Leu1106) amino-terminally fused to a GST-HIS6-Thrombin cleavage site. The proteins were purified by one-step affinity chromatography using glutathione-agarose. An anti-phosphotyrosine monoclonal antibody, P-Tyr-100, was used to detect phosphorylation of biotinylated substrate peptides (VEGFR2, Biotin-Gastrin Precursor (Tyr87); PDGFR-β, Biotinylated-FLT3 (Tyr589)). Enzymatic activity was tested in 60 mM HEPES, 5 mM MgCl2 5 mM MnCl2 200 μM ATP, 1.25 mM DTT, 3 μM Na3VO4, 1.5 mM peptide, and 50 ng EGF Receptor Kinase. Bound antibody was detected using the DELFIA system (PerkinElmer, Wellesley, Mass.) consisting of DELFIA® Europium-labeled Anti-mouse IgG (PerkinElmer, #AD0124), DELFIA® Enhancement Solution (PerkinElmer, #1244-105), and a DELFIA® Streptavidin coated, 96-well Plate (PerkinElmer, AAAND-0005). Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm.

Test compounds were dissolved in dimethylsulphoxide (DMSO) to give a 20 mM working stock concentration. Each assay was setup as follows: Added 100 μl of 10 mM ATP to 1.25 ml 6 mM substrate peptide. Diluted the mixture with dH₂0 to 2.5 ml to make 2×ATP/substrate cocktail ([ATP]=400 mM, [substrate]=3 mM). Immediately transfer enzyme from −80° C. to ice. Allowed enzyme to thaw on ice. Microcentrifuged briefly at 4° C. to bring liquid to the bottom of the vial. Returned immediately to ice. Added 10 μl of DTT (1.25 mM) to 2.5 ml of 4×HTScan™ Tyrosine Kinase Buffer (240 mM HEPES pH 7.5, 20 mM MgCl₂, 20 mM MnCl, 12 mM NaVO₃) to make DTT/Kinase buffer. Transfer 1.25 ml of DTT/Kinase buffer to enzyme tube to make 4× reaction cocktail ([enzyme]=4 ng/μL in 4× reaction cocktail). Incubated 12.5 μl of the 4× reaction cocktail with 12.5 μl/well of prediluted compound of interest (usually around 10 μM) for 5 minutes at room temperature. Added 25 μl of 2×ATP/substrate cocktail to 25 μl/well preincubated reaction cocktail/compound. Incubated reaction plate at room temperature for 30 minutes. Added 50 μl/well Stop Buffer (50 mM EDTA, pH 8) to stop the reaction. Transferred 25 μl of each reaction and 75 μl dH₂O/well to a 96-well streptavidin-coated plate and incubated at room temperature for 60 minutes. Washed three times with 200 μl/well PBS/T (PBS, 0.05% Tween-20). Diluted primary antibody, Phospho-Tyrosine mAb (P-Tyr-100), 1:1000 in PBS/T with 1% bovine serum albumin (BSA). Added 100 μl/well primary antibody. Incubated at room temperature for 60 minutes. Washed three times with 200 μl/well PBS/T. Diluted Europium labeled anti-mouse IgG 1:500 in PBS/T with 1% BSA. Added 100 μl/well diluted antibody. Incubated at room temperature for 30 minutes. Washed five times with 200 μl/well PBS/T. Added 100 μl/well DELFIA® Enhancement Solution. Incubated at room temperature for 5 minutes. Detected 615 nm fluorescence emission with appropriate Time-Resolved Plate Reader.

(b) An In Vitro Assay which Determines the Ability of a Test Compound to Inhibit HDAC Enzymatic Activity.

HDAC inhibitors were screened using an HDAC fluorimetric assay kit (AK-500, Biomol, Plymouth Meeting, Pa.). Test compounds were dissolved in dimethylsulphoxide (DMSO) to give a 20 mM working stock concentration. Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm. Each assay was setup as follows: Defrosted all kit components and kept on ice until use. Diluted HeLa nuclear extract 1:29 in Assay Buffer (50 mM Tris/Cl, pH 8.0, 137 mM NaCl, 2.7 mM KCl, 1 mM MgCl2). Prepared dilutions of Trichostatin A (TSA, positive control) and tested compounds in assay buffer (5× of final concentration). Diluted Fluor de Lys™ Substrate in assay buffer to 100 uM (50 fold=2× final). Diluted Fluor de Lys™ developer concentrate 20-fold (e.g. 50 μl plus 950 μl Assay Buffer) in cold assay buffer. Second, diluted the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Added Assay buffer, diluted trichostatin A or test inhibitor to appropriate wells of the microtiter plate. Added diluted HeLa extract or other HDAC sample to all wells except for negative controls. Allowed diluted Fluor de Lys™ Substrate and the samples in the microtiter plate to equilibrate to assay temperature (e.g. 25 or 37° C. Initiated HDAC reactions by adding diluted substrate (25 μl) to each well and mixing thoroughly. Allowed HDAC reactions to proceed for 1 hour and then stopped them by addition of Fluor de Lys™ Developer (50 μl). Incubated plate at room temperature (25° C.) for 10-15 min. Read samples in a microtiter-plate reading fluorimeter capable of excitation at a wavelength in the range 350-380 nm and detection of emitted light in the range 440-460 nm.

The following TABLE 2-B lists compounds representative of the invention and their activity in HDAC, VEGFR2 and PDGFR assays. In these assays, the following grading was used: I≧10 μM, 10 μM>II>1 μM, 1 μM>III>0.1 μM, and IV≦0.1 μM for IC₅₀.

TABLE 2-B

| Compound No. | HDAC | VEGFR2 | PDGFR |
|---|---|---|---|
| 2 | III | IV | III |
| 3 | II | IV | IV |
| 4 | III | IV | IV |
| 8 | III | | |
| 9 | I | IV | |
| 14 | III | IV | |
| 15 | IV | IV | III |
| 16 | IV | IV | III |

TABLE 3-A

| | |
|---|---|
| | (IV) |
| 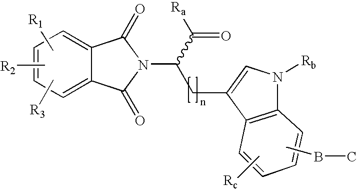 | |
| | (V) |
| 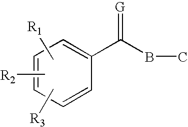 | |

SECTION 3:

| Com- | |
|---|---|
| pound | |
| No. | Structure |
| 1 | 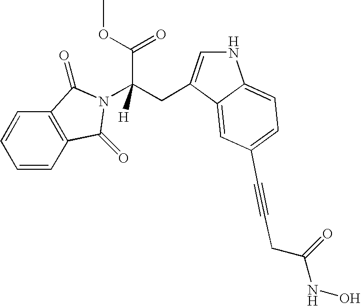 |
| 2 | 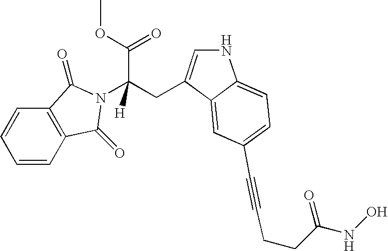 |
| 3 | 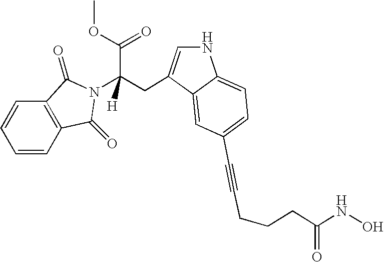 |
| 4 | 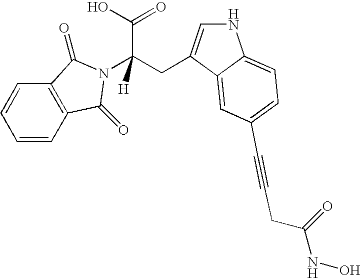 |
| 5 | 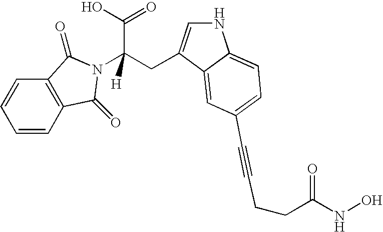 |
| 6 | 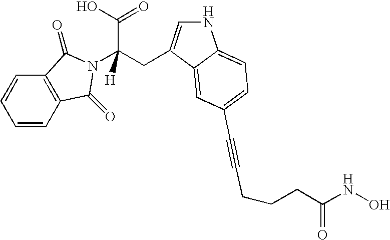 |
| 7 | 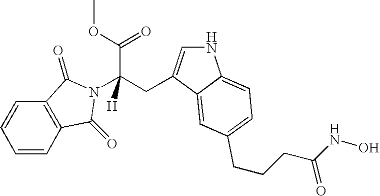 |
| 8 | 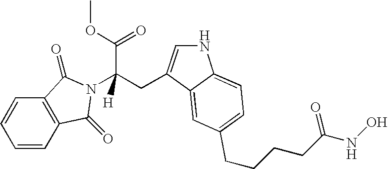 |
| 9 | 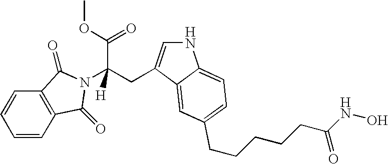 |
| 10 | 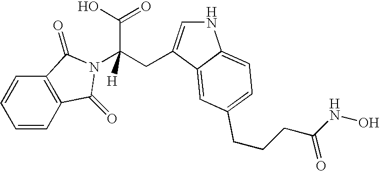 |
| 11 | 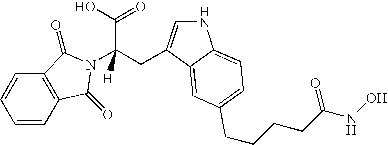 |
| 12 | 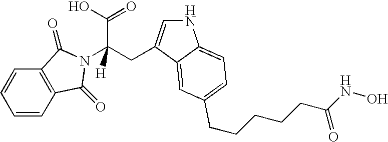 |
| 13 | 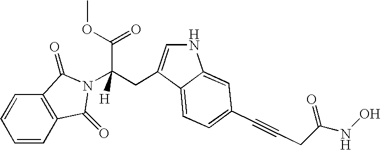 |
| 14 | 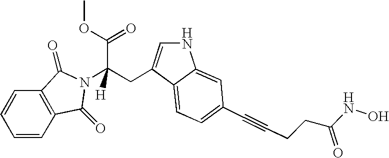 |
| 15 | 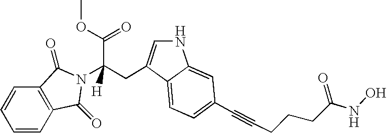 |
| 16 | 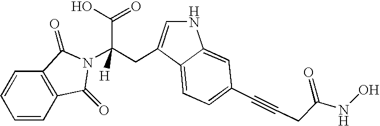 |
| 17 | 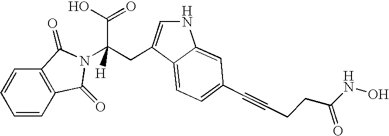 |
| 18 | 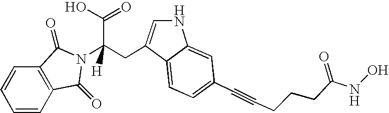 |
| 19 | 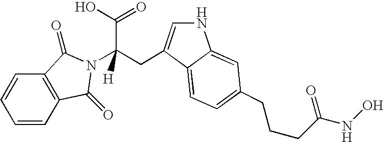 |
| 20 | 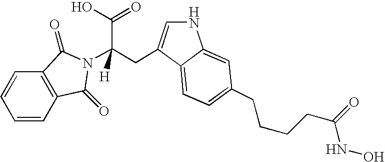 |
| 21 | 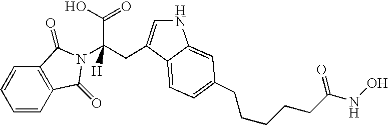 |
| 22 | 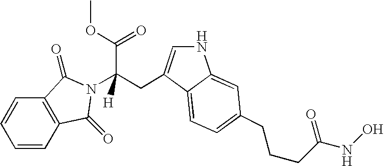 |
| 23 | 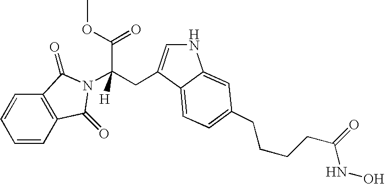 |
| 24 | 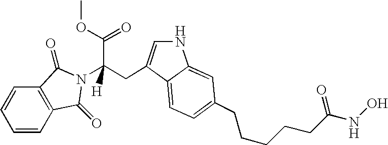 |
| 25 | 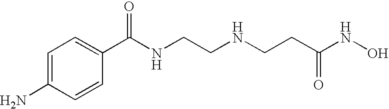 |
| 26 | 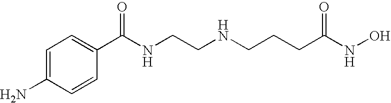 |
| 27 | 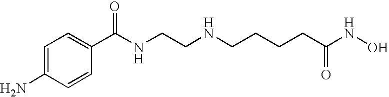 |
| 28 | 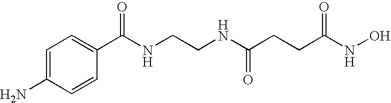 |
| 29 | 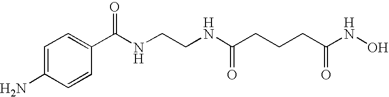 |
| 30 | 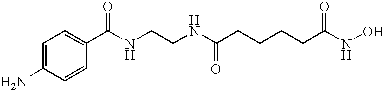 |
| 31 | 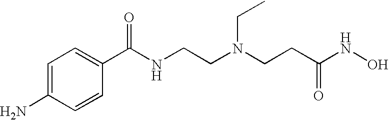 |
| 32 | 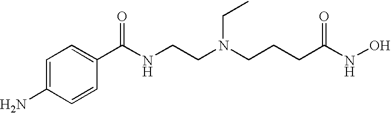 |
| 33 | 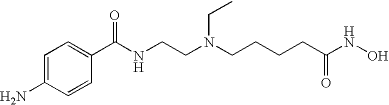 |
| 34 | 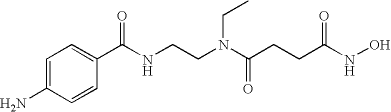 |
| 35 | 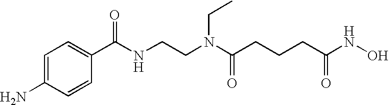 |
| 36 | 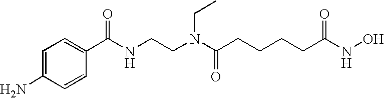 |
| 37 | 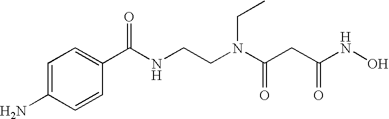 |
| 38 | 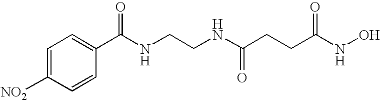 |
| 39 | 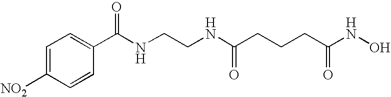 |

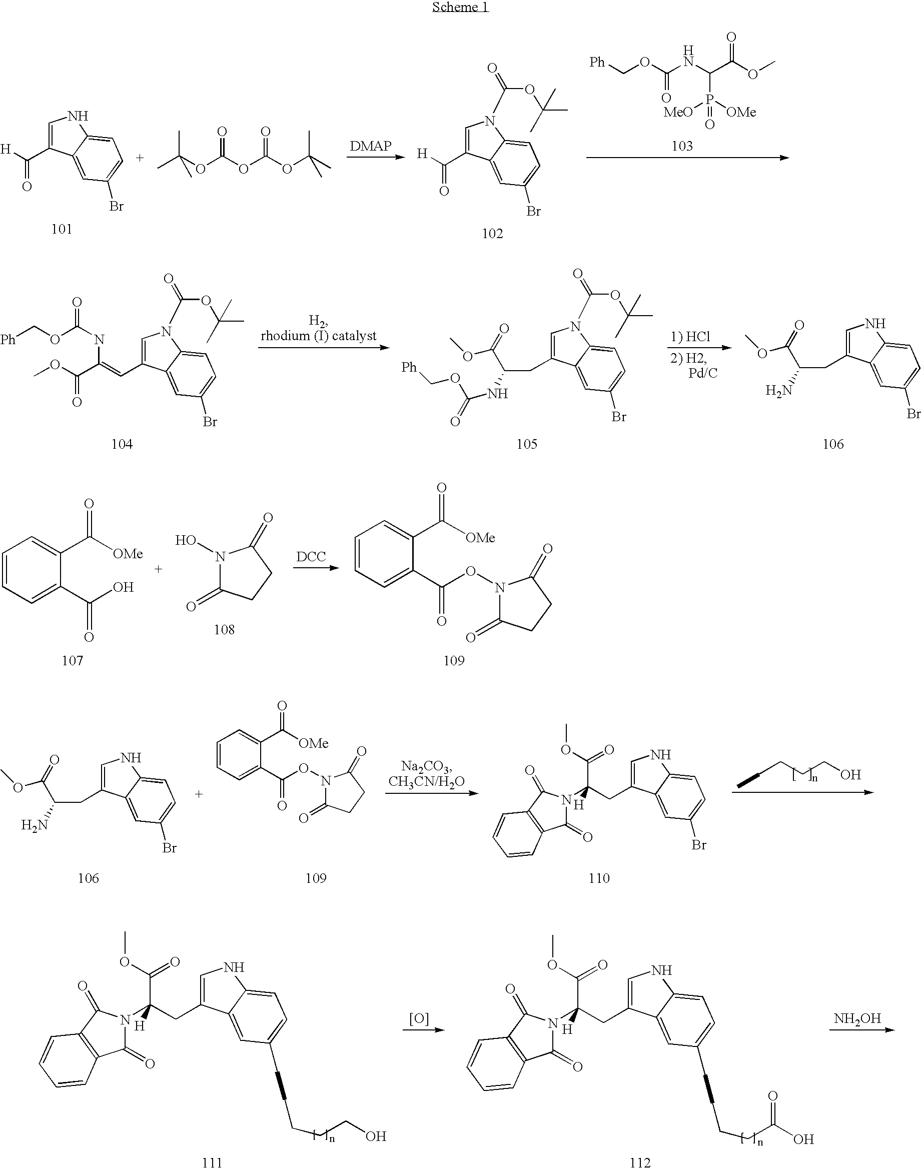

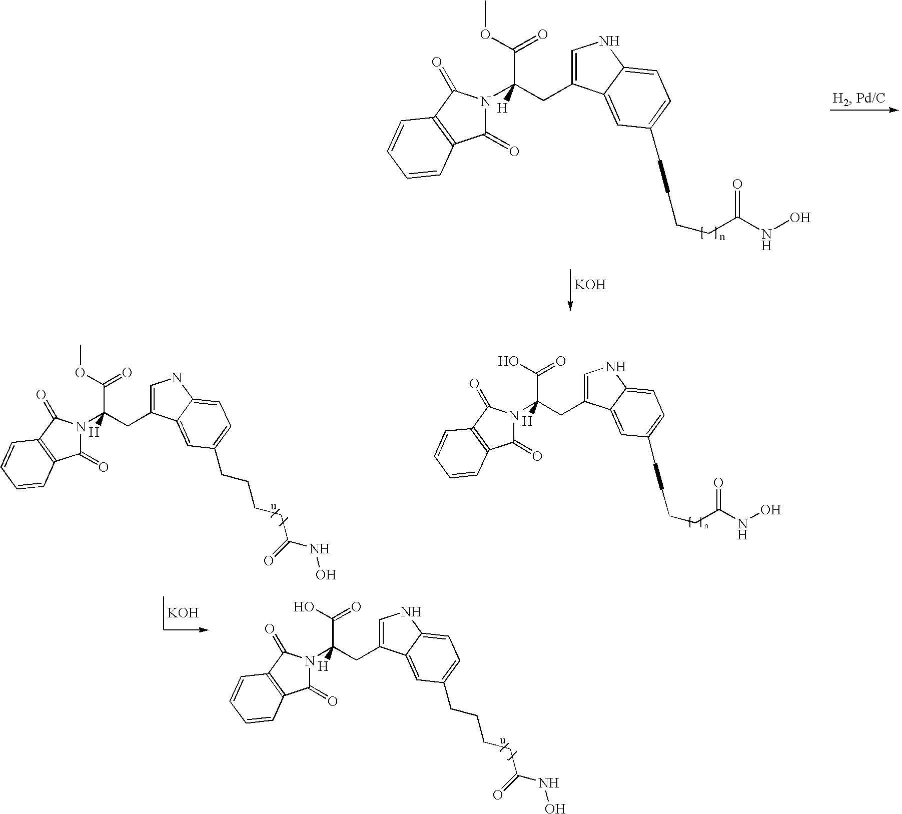

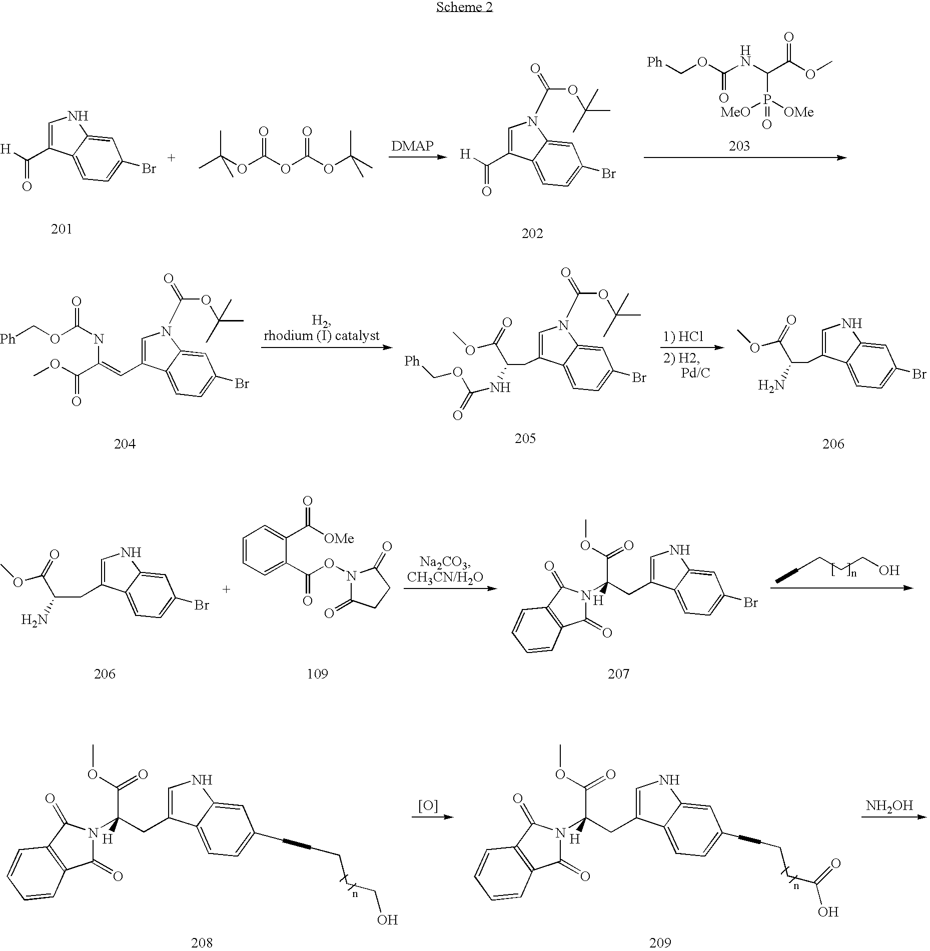

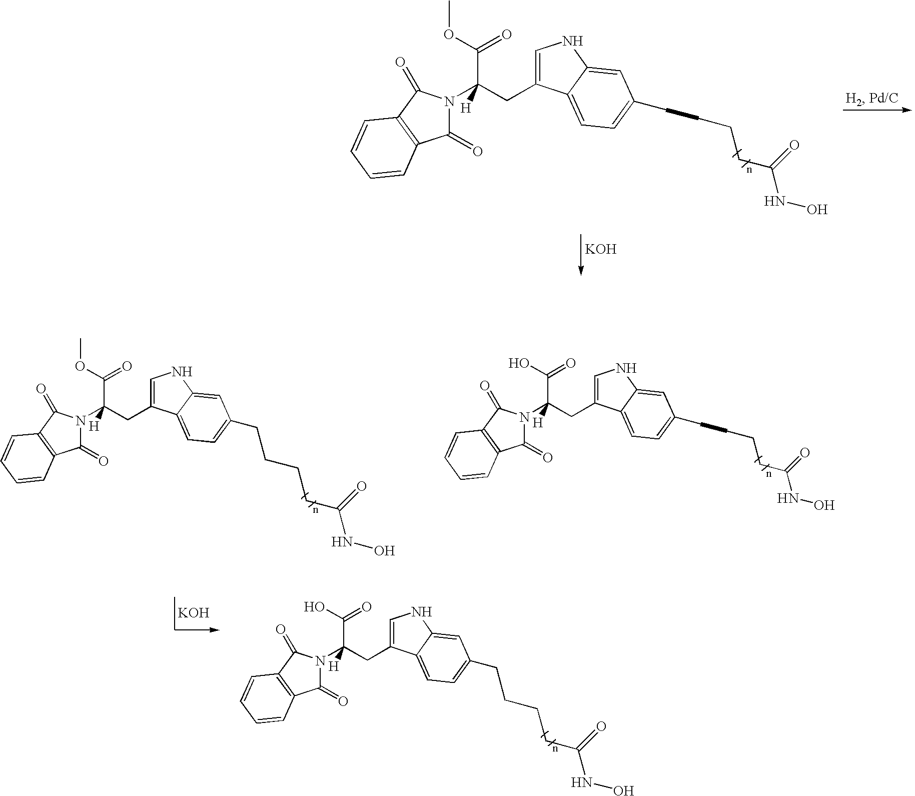

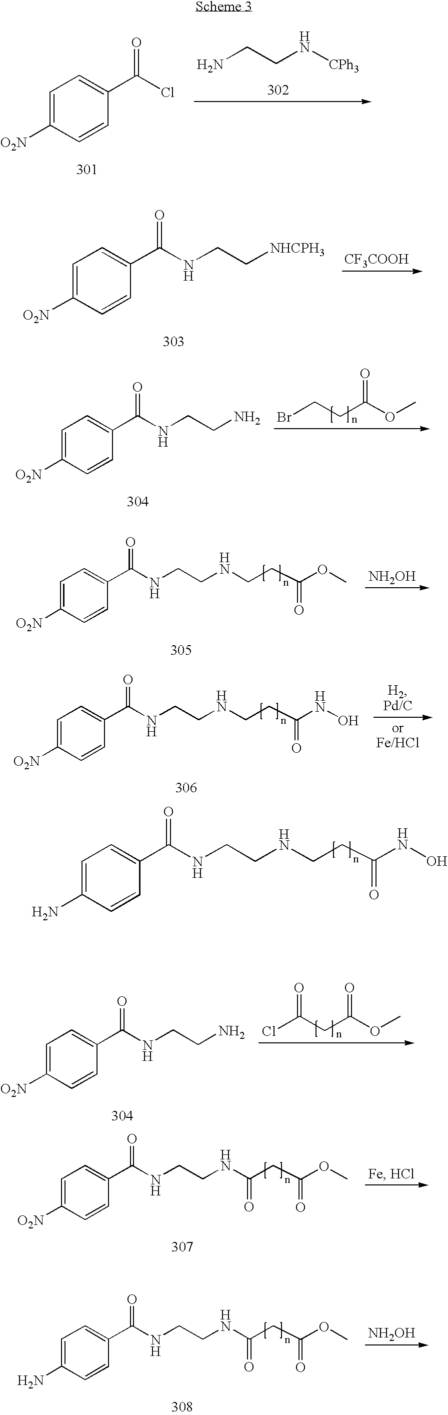

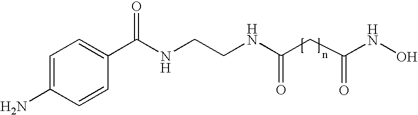

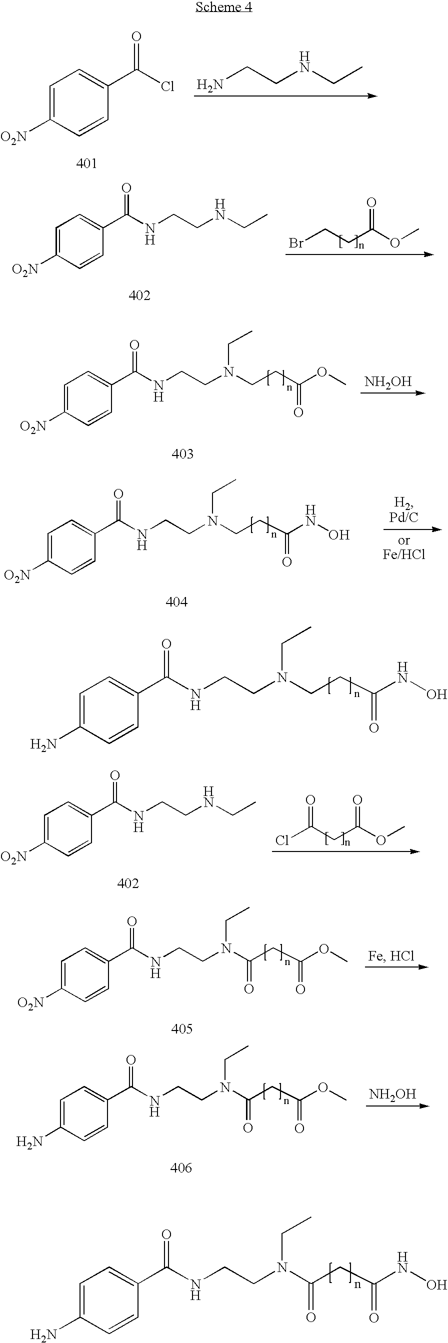

### Example 1

### Preparation of N-hydroxy-4-(2-(4-aminobenzamido)-ethylcarbamoyl)butanamide (Compound 29)

### Step 1a. N¹-Tritylethane-1,2-diamine (compound 302)

To a mixture of ethylenediamine (30 g, 0.5 mol) and triethylamine (50 g, 0.5 mol) in CH₂Cl₂ (300 mL) was added dropwise the solution of chlorotriphenylmethane (28.0 g, 0.1 mol) in CH₂Cl₂ (200 mL) over 2 h. The mixture was then stirred at room temperature overnight. The reaction mixture was washed with water (200 mL×4), dried over Na₂SO₄, concentrated to give the compound 302 (25 g, 83.3%). ¹H NMR (CDCl₃) δ 7.14-7.49 (m, 15H), 3.78 (br, 2H), 2.87 (d, 2H), 2.35 (d, 2H). LC-MS: m/z 303 (M+1).

### Step 1b. 4-Nitro-N-(2-(tritylamino)ethyl)benzamide (compound 303)

To a solution of 302 (1.4 g, 4.6 mmol) in CH₂Cl₂ (100 mL) containing triethylamine (505 mg, 5 mmol) was added dropwise the solution of 4-nitrobenzoyl chloride 801 (872 mg, 4.7 mmol) in CH₂Cl₂ (20 mL). The mixture was stirred for 2 h and diluted with CH₂Cl₂ (200 mL), washed with water, dried and concentrated to afford the compound 303 as a solid (1.8 g, 87% yield). The product was used directly in the next step. ¹H NMR (CDCl₃) δ 8.31 (d, 2H), 7.93 (d, 2H), 7.19-7.46 (m, 15H), 3.55-3.57 (m, 2H), 2.44-2.46 (m, 2H). LC-MS: m/z 452 (M+1).

### Step 1C. N-(2-Aminoethyl)-4-nitrobenzamide (compound 304)

To a stirred solution of compound 303 (18.0 g, 0.04 mol) in CH₂Cl₂ (200 mL) at room temperature was added trifluoroacetic acid (8.0 mmol) dropwise. After stirring for 0.5 h, a plenty of precipitates appeared. The solution was filtered and the resulting solid was washed with CH₂Cl₂ (100 mL×2) to afford the product 304 as a white solid (12.0 g, 93.7% yield). ¹H NMR (D₂O) δ 8.20 (d, 2H), 7.84 (d, 2H), 3.60 (t, 2H), 3.15 (t, 2H). LC-MS: m/z 210 (M+1).

### Step 1d. Methyl 4-(2-(4-nitrobenzamido)ethylcarbamoyl)butanoate (compound 307-29)

To a solution of 304 (1.92 g, 6 mmol) in CH₂Cl₂ (30 mL) containing triethylamine (4 mL) was added the solution of 4-(methylperoxy)pent-4-enoylchloride in CH₂Cl₂ (5 mL). The mixture was then stirred at room temperature for 1 h and diluted with 200 mL of acetate ethyl. The resulting mixture was washed with water (50 mL×3), dried, and concentrated to afford the product 307-29 as a white solid. ¹H NMR (d⁶-DMSO) δ 8.78-8.82 (m, 1H), 8.29 (d, 2H), 8.04 (d, 2H), 7.94-7.96 (m, 1H), 3.55 (s, 3H), 2.26 (t, 2H), 2.08 (t, 2H), 1.66-1.72 (m, 2H). ¹H NMR (CD₃OD) δ 8.31 (d, 2H), 8.01 (d, 2H), 3.62 (s, 3H), 3.49-3.51 (m, 2H), 3.40-3.43 (m, 2H), 2.32 (t, 2H), 2.23 (t, 2H), 1.84-1.89 (m, 2H). LC-MS: m/z 338 (M+1).

### Step 1e. Methyl 4-(2-(4-aminobenzamido)ethylcarbamoyl)butanoate (compound 308-29)

A mixture was prepared containing compound 307-29 (674 mg, 2 mmol), iron power (1.12 g, 20 mmol), EtOH (15 mL) and water (0.5 mL). To this mixture was added 0.5 mL of concentrated HCl at room temperature. Then the resulting mixture was heated to reflux. The reaction was stirred until the starting material disappeared monitored by TLC. The reaction mixture was cooled to room temperature and filtered. The filtrate was concentrated to give a residue which was purified by column chromatography on silica gel (ethyl acetate) to afford the product 308-29 as a white solid (240 mg, 39% yield). ¹H NMR (6-DMSO) δ 7.97-8.01 (m, 1H), 7.89-7.92 (m, 1H), 7.53 (d, 2H), 6.51 (d, 2H), 6.57 (s, 2H), 3.57 (s, 1H), 3.15-3.24 (m, 4H), 2.29 (t, 2H), 2.09 (t, 2H), 1.70-1.75 (m, 2H). LC-MS: m/z 308 (M+1).

### Step 1f. N-hydroxy-4-(2-(4-aminobenzamido)ethylcarbamoyl)butanamide (compound 29)

Preparation of the solution of hydroxylamine in methanol: hydroxylamine hydrochloride (4.67 g, 67 mmol) was dissolved in methanol (24 ml) made to solution A. Potassium hydroxide (5.61 g, 100 mmol) was dissolved in methanol (14 ml) made to solution B. The solution A was cooled to 0° C., and solution B was added into solution A dropwise. The mixture was stirred for 30 minutes at 0° C., and was placed long time at low temperature. The precipitate was isolated to afford the solution of hydroxylamine in methanol.

To a flask containing compound 308-29 (40 mg, 0.13 mmol) was added above solution of hydroxyamine in methanol (0.5 mL). The mixture was stirred for 5 min. Then it was adjusted to PH 8 using concentrated HCl. The desired product 29 was obtained after prep-TLC separation (dichloromethane: methanol=4:1) (40 mg, 99% yield). ¹H NMR (DMSO-d₆) δ 10.16 (s, 1H), 8.64 (s, 1H), 8.02 (m, 1H), 7.90 (m, 1H), 7.52 (d, 2H), 6.51 (d, 2H), 5.56 (s, 2H), 3.12-3.23 (m, 4H), 2.03 (t, 2H), 3.14 (t, 2H), 1.65-1.70 (m, 2H). LC-MS: m/z 309 (M+1). Mp: 158.9-159.8° C.

### Example 2

### Preparation of 4-amino-N-(2-(ethyl(3-(hydroxyamino)-3-oxopropyl)amino)ethyl)benzamide (compound 31)

### Step 2a. Methyl 3-(ethyl(2-(4-nitrobenzamido)ethyl)amino)propanoate (compound 403-31)

To a solution of N-(2-(ethylamino)ethyl)-4-nitrobenzamide (402) (1.19 g, 5 mmol) in DMF (10 ml) was added K₂CO₃ (1.38 g, 10 mmol), and then methyl 3-bromopropanoate (994 mg, 6 mmol) was added to the mixture. The mixture was stirred for 5 h at 40° C. and then the solid was removed by filtration. The solvent was removed under reduced pressure. The residue was purified on column chromatography to give 1380 mg of pure product 403-31 (83% yield). ¹H NMR (CDCl₃) δ 8.292 t, 1H), 8.262 (t, 1H), 8.081 (t, 1H), 8.051 (t, 1H), 3.635 (s, 3H), 3.56 (m, 2H), 2.786 (t, 2H), 2.666 (t, 2H), 2.539 (m, 4H), 0.978 (t, 3H); LC-MS: 323 (M+1).

### Step 2b. Methyl 3-((2-(4-aminobenzamido)ethyl)(ethyl)amino)propanoate (compound 404-31)

To a flask containing compound 403-31 (200 mg, 0.62 mmol), iron power (364 mg, 6.5 mmol), methanol (10 mL) and water (0.5 mL) was added 1 drop of concentrated hydrochloride acid. The resulting mixture was refluxed for 3 h, and then cooled to room temperature and filtered. The filtrate was concentrated and the residue was purified by column chromatography on silica gel (ethyl acetate) to afford 404-31 as a sticky liquid (141 mg, 77.5% yield). ¹H NMR (CDCl₃) δ 7.698 (m, 2H), 6.670 (m, 2H), 3.629 (s, 3H), 3.524 (t, 2H), 2.693 (m, 8H), 1.235 (t, 3H); LC-MS: 295 (M+1).

### Step 2c. 4-amino-N-(2-(ethyl(3-(hydroxyamino)-3-oxopropyl)amino)ethyl)benzamide (compound 31)

To a flask containing compound 404-31 (118 mg, 0.4027 mmol) was added the fresh solution of hydroxyamine (2.42 mmol) in methanol (1.34 ml). The mixture was stirred for 5 min and then was adjusted to PH 8 using concentrated hydrochloride acid diluted with methanol. The crude product was purified by column chromatography to afford 76 mg of compound 31 (64.5% yield). ¹H NMR (DMSO-d₆) δ 10.484 (s, 1H), 8.765 (s, 1H), 7.983 (s, 1H), 7.567 (m, 2H), 6.539 (d, 2H), 5.597 (s, 2H), 2.866 (s, 2H), 2.694 (s, 4H), 2.209 (t, 2H), 1.012 (t, 3H); LC-MS: 293 (M+1).

### Example 3

### Preparation of 4-amino-N-(2-(ethyl(4-(hydroxyamino)-4-oxobutyl)amino)ethyl)benzamide (compound 32)

### Step 3a. ethyl 4-(ethyl(2-(4-nitrobenzamido)ethyl)amino)butanoate (compound 403-32)

The title compound 403-32 was prepared from 402 using a procedure similar to that described for compound 403-31 (Example 2) with 51.8% yield. ¹H NMR (CDCl₃) δ 8.28 (d, 2H), 8.04 (d, 2H), 4.08 (m, 2H), 3.52 (m, 2H), 2.66 (t, 2H), 2.52 (m, 4H), 1.81 (m, 2H), 1.22 (t, 3H), 1.00 (t, 3H); LC-MS: 352 (M+1).

### Step 3b. Ethyl 4-((2-(4-aminobenzamido)ethyl)(ethyl)amino)butanoate (compound 404-32)

The title compound 404-32 was prepared from 403-32 using a procedure similar to that described for compound 404-31 (Example 2) with 52.5% yield. ¹H NMR (CDCl₃) δ 7.658 (d, 2H), 6.656 (d, 2H), 4.109 (m, 2H), 3469 (m, 2H), 2.545 (t, 6H), 2.326 (t, 2H), 1.816 (m, 2H), 1.200 (t, 3H), 1.001 (t, 3H); LC-MS: 322 (M+1).

### Step 3c. 4-amino-N-(2-(ethyl(4-(hydroxyamino)-4-oxobutyl)amino)ethyl)benzamide (compound 32)

The title compound 32 was prepared from 404-32 using a procedure similar to that described for compound 31 (Example 2) with 63.3% yield (Example 2). ¹H NMR (Methanol-d₆) δ 7.61 (d, 2H), 6.67 (d, 2H), 3.53 (t, 3H), 2.85 (m, 6H), 2.18 (t, 2H), 1.89 (m, 2H), 1.16 (t, 3H); LC-MS: 309 (M+1).

### Example 4

### Preparation of N-hydroxy-5-(ethyl(2-(4-aminobenzamido)-ethyl)amino)-5-oxopentanamide (compound 35)

### Step 4a. N-(2-(ethylamino)ethyl)-4-nitrobenzamide (compound 402)

To a mixture of N-ethylethylenediamine (13.2 g, 160 mmol) and triethylamine (32 g, 320 mmol) in diethylether (200 mL) was added dropwise the solution of 4-nitrobenzoyl chloride 901 (15 g, 81.1 mmol) in diethylether (800 mL) at 0° C. The mixture was then stirred for 15 min at this temperature. The reaction mixture was filtered and the filtering cookie was suspended in water (400 mL), 10% of hydrochloride acid was added to adjust PH=3. the resulting acidified mixture was extracted with ethyl acetate (80 mL×3) and 15% NaOH was then added to the water phase up to PH=8. This alkali solution was extracted with washed with ethyl acetate (80 mL×3), the combined organic layer was dried over anhydrous Na₂SO₄, concentrated to give the compound 402 (7.5 g, 39%) as a white solid. ¹H NMR (CDCl₃) δ 8.28 (d, 2H), 7.95 (d, 2H), 6.96 (br, 1H), 3.53 (t, 2H), 2.89 (t, 2H), 2.69 (q, 2H), 1.13 (t, 2H). LC-MS: 238 (M+1).

### Step 4b. Methyl-5-(ethyl(2-(4-nitrobenzamido)ethyl)amino)-5-oxopentanoate (compound 405-35)

To a solution of 5-methoxy-5-oxopentanoic acid (0.9 g, 6 mmol) in CH₂Cl₂ (15 mL) was added oxalyl dichloride (0.93 g, 7.2 mmol) dropwise, and then a drop of DMF was added to the mixture as catalyst. The mixture was stirred for 1.5 h at room temperature and then was concentrated under reduced pressure till the excess oxalyl dichloride was absolutely removed. The solution of the resultant methyl-5-chloro-5-oxopentanoate in CH₂Cl₂ (5 mL) was dropwise added to a solution of compound 402 (0.72 g, 3 mmol) and triethylamine (0.61 g, 6 mmol) in CH₂Cl₂ (10 mL) at room temperature. The mixture was stirred at room temperature overnight, washed with water, dried over anhydrous Na₂SO₄, and concentrated. The crude product was separated by flash column chromatography (50% ethyl acetate/petroleum) to afford the 0.74 g of 405-35 as a white solid. ¹H NMR (CDCl₃) δ 8.28 (d, 2H), 8.01 (d, 2H), 3.65 (m, 7H), 3.39 (q, 2H), 2.43 (t, 2H), 2.38 (t, 2H), 1.95 (m, 2H), 1.23 (t, 3H); LC-MS: 366 (M+1).

### Step 4c. Methyl-5-(ethyl(2-(4-aminobenzamido)ethyl)amino)-5-oxopentanoate (compound 406-35)

To a flask containing compound 405-35 (0.74 g, 2 mmol), iron power (1.12 g, 20 mmol), methanol (15 mL) and water (0.5 mL) was added 4 drop of concentrated hydrochloride acid. The resulting mixture was refluxed for 3 h, and then cooled to room temperature and filtered. The filtrate was concentrated and the residue was purified by column chromatography on silica gel (ethyl acetate) to afford 406-35 as a sticky liquid (0.56 g, 83% yield). ¹H NMR (methanol-d₄) δ 7.99 (d, 1H), 7.90 (d, 1H), 3.74 (s, 3H), 3.67 (d, 4H), 3.62 (q, 2H), 2.45 (m, 2H), 2.21 (m, 2H), 1.91 (m, 2H), 1.19 (m, 3H); LC-MS: 336 (M+1).

### Step 4d. N-hydroxy-5-(ethyl(2-(4-aminobenzamido)ethyl)amino)-5-oxopentanamide (compound 35)

To a flask containing compound 406-35 (121 mg, 0.36 mmol) was added the fresh solution of hydroxyamine (2.16 mmol) in methanol (1.2 mL). The mixture was stirred for 5 min and then was adjusted to PH 8 using concentrated hydrochloride acid diluted with methanol. The crude product was purified by HPLC to afford 40 mg of compound 35. ¹H NMR (DMSO-d₆) δ 10.23 (s, 1H), 8.62 (s, 1H), 8.07-8.19 (m, 1H), 7.52 (m, 2H), 5.59 (d, 2H), 3.21-3.51 (m, 6H), 2.27 (m, 2H), 1.95 (m, 2H), 1.70 (m, 2H), 0.99-1.08 (m, 3H); LC-MS: 337 (M+1).

### Example 5

### Synthesis of N¹-(2-(4-aminobenzamido)ethyl)-N¹-ethyl-N⁴-hydroxysuccinamide (compound 34)

### Step 5a. Methyl 4-(ethyl(2-(4-nitrobenzamido)ethyl)amino)-4-oxobutanoate (compound 405-34)

The title compound 405-34, a pale yellow solid, was prepared from 902 and 4-(methylperoxy)-4-oxobutanoic acid using a procedure similar to that described for compound 405-35 (Example 4) with 77% yield. ¹H NMR (CDCl₃) δ 8.26 (d, 2H), 7.97 (d, 2H), 3.64 (s, 3H), 3.65 (m, 4H), 3.48 (q, 2H), 2.68 (s, 4H), 1.25 (t, 3H); LC-MS: 352 (M+1).

### Step 5b. Methyl 4-((2-(4-aminobenzamido)ethyl)(ethyl)amino)-4-oxobutanoate (compound 403-34)

The title compound 406-34, a white sticky liquid, was prepared from 405-34 using a procedure similar to that described for compound 406-35 (Example 4). ¹H NMR (methanol-d₄) δ 7.58 (d, 2H), 6.55 (d, 2H), 3.69 (s, 3H), 3.58 (m, 4H), 3.46 (q, 2H), 2.70 (t, 4H), 1.19 (m, 3H); LC-MS: 322 (M+1).

### Step 5c. N¹-(2-(4-aminobenzamido)ethyl)-N¹-ethyl-N⁴-hydroxysuccinamide (compound 34)

The title compound 34, a white powder, was prepared from 406-34 using a procedure similar to that described for compound 35 (Example 4). ¹H NMR (DMSO-d₆) δ 10.34 (d, 1H), 8.65 (d, 1H), 8.05 (m, 1H), 7.53 (m, 2H), 6.59 (m, 2H), 5.58 (d, 2H), 3.21-3.48 (m, 6H), 2.49 (m, 2H), 2.20 (m, 2H), 0.97-1.13 (m, 3H); LC-MS: 323 (M+1).

### Example 6

### Preparation of N¹-(2-(4-aminobenzamido)ethyl)-N¹-ethyl-N-hydroxyadipamide (compound 36)

### Step 6a. Methyl 6-(ethyl(2-(4-nitrobenzamido)ethyl)amino)-6-oxohexanoate (compound 405-36)

The title compound 405-36, a white crystal, was prepared from 902 using a procedure similar to that described for compound 405-35 (Example 4) with 81% yield. ¹H NMR (CDCl₃) δ 8.26 (d, 2H), 7.98 (d, 2H), 3.65 (s, 3H), 3.64 (m, 4H), 3.47 (q, 2H), 2.35 (m, 4H), 1.66 (m, 4H), 1.24 (t, 3H); LC-MS: 380 (M+1).

### Step 6b. Methyl 6-((2-(4-aminobenzamido)ethyl)(ethyl)amino)-6-oxohexanoate (compound 406-36)

The title compound 406-36, a white sticky liquid, was prepared from 405-36 using a procedure similar to that described for compound 405-35 (Example 4). ¹H NMR (methanol-d₄) δ 7.67 (d, 2H), 6.58 (d, 2H), 3.63 (s, 3H), 3.61 (m, 4H), 3.46 (q, 2H), 2.35 (m, 2H), 2.12 (m, 2H), 1.69 (m, 4H), 1.20 (t, 3H); LC-MS: 350 (M+1).

### Step 6c. N¹-(2-(4-aminobenzamido)ethyl)-N¹-ethyl-N⁶-hydroxyadipamide (compound 36)

The title compound 36, a white powder, was prepared from 406-36 using a procedure similar to that described for compound 35 (Example 4). ¹H NMR (DMSO-d₆) δ 8.20 (m, 1H), 7.55 (m, 2H), 6.53 (m, 2H), 5.58 (d, 2H), 3.25-3.48 (m, 6H), 2.26 (m, 2H), 1.88 (m, 2H), 1.45 (m, 2H), 0.98-1.11 (m, 3H); LC-MS: 351 (M+1).

### Example 7

### Preparation of N-hydroxy-3-((2-(4-aminobenzamido)-ethyl)(ethyl)amino)-3-oxopropanamide (compound 37)

### Step 7a. Methyl 3-(ethyl(2-(4-nitrobenzamido)ethyl)amino)-3-oxopropanoate (compound 405-37)

The title compound 405-37, a yellow solid, was prepared from 902 and 3-methoxy-3-oxopropanoic acid using a procedure similar to that described for compound 405-35 (Example 4) with 80% yield. ¹H NMR (CDCl₃) δ 8.27 (d, 2H), 8.00 (d, 2H), 4.15 (s, 3H), 3.69 (s, 4H), 3.51 (s, 2H), 3.45 (q, 2H), 1.23 (t, 3H); LC-MS: 338 (M+1).

### Step 7b. Methyl 3-((2-(4-aminobenzamido)ethyl)(ethyl)amino)-3-oxopropanoate (compound 406-37)

The title compound 406-37, a white sticky liquid, was prepared from 905-37 using a procedure similar to that described for compound 406-35 (Example 4). ¹H NMR (methanol-d₄) δ 7.57 (d, 2H), 6.52 (d, 2H), 3.89 (s, 3H), 3.72 (d, 4H), 3.54 (s, 2H), 1.20 (m, 3H); LC-MS: 308 (M+1).

### Step 7c. N-hydroxy-3-((2-(4-aminobenzamido)ethyl)(ethyl)amino)-3-oxopropanamide (compound 37)

The title compound 37, a white powder, was prepared from 406-37 using a procedure similar to that described for compound 35 (Example 4). ¹H NMR (DMSO-d₆) δ 10.51 (s, 1H), 8.89 (s, 1H), 8.11 (m, 1H), 7.53 (d, 2H), 6.52 (m, 2H), 5.59 (d, 2H), 3.27-3.51 (m, 6H), 3.14 (d, 2H), 0.99-1.14 (m, 3H); LC-MS: 309 (M+1).

### Biological Assays:

As stated hereinbefore the derivatives defined in the present invention possess anti-proliferative activity. These properties may be assessed, for example, using one or more of the procedures set out below:

(a) An In Vitro Assay which Determines the Ability of a Test Compound to Inhibit HDAC Enzymatic Activity.

HDAC inhibitors were screened using an HDAC fluorimetric assay kit (AK-500, Biomol, Plymouth Meeting, Pa.). Test compounds were dissolved in dimethylsulphoxide (DMSO) to give a 20 mM working stock concentration. Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm.

Each assay was setup as follows: Defrosted all kit components and kept on ice until use. Diluted HeLa nuclear extract 1:29 in Assay Buffer (50 mM Tris/Cl, pH 8.0, 137 mM NaCl, 2.7 mM KCl, 1 mM MgCl2). Prepared dilutions of Trichostatin A (TSA, positive control) and tested compounds in assay buffer (5× of final concentration). Diluted Fluor de Lys™ Substrate in assay buffer to 100 uM (50 fold=2× final). Diluted Fluor de Lys™ developer concentrate 20-fold (e.g. 50 μl plus 950 μl Assay Buffer) in cold assay buffer. Second, diluted the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Added Assay buffer, diluted trichostatin A or test inhibitor to appropriate wells of the microtiter plate. Added diluted HeLa extract or other HDAC sample to all wells except for negative controls. Allowed diluted Fluor de Lys™ Substrate and the samples in the microtiter plate to equilibrate to assay temperature (e.g. 25 or 37° C. Initiated HDAC reactions by adding diluted substrate (25 μl) to each well and mixing thoroughly. Allowed HDAC reactions to proceed for 1 hour and then stopped them by addition of Fluor de Lys™ Developer (50 μl). Incubated plate at room temperature (25° C.) for 10-15 min. Read samples in a microtiter-plate reading fluorimeter capable of excitation at a wavelength in the range 350-380 nm and detection of emitted light in the range 440-460 nm.

(b) An In Vitro Assay which Determined the Ability of a Test Compound to Inhibit DNMT Activity.

DNMT inhibitors are screened using methylation specific PCR (MSP). Test compounds are dissolved in dimethylsulphoxide (DMSO) to give a 20 mM working stock concentration. HT-29 colon adenocarcinoma cells are plated in 6 well plates and treated for 72 hours with test compound or 2.5 μM 5-Aza-2′-deoxycytidine, replacing the media daily. DNA is harvested from cells after 72 hours using a non-organic DNA extraction kit (S4520, Chemicon International, Temecula, Calif.). Bisulfite chemical modification is achieved using the CpCenome DNA Modification Kit (S7820, Chemicon International, Temecula, Calif.). In a screwcap 1.5-2.0 mL microcentrifuge tube are added 7.0 μL 3M NaOH to 1.0 μg DNA in 100 μL of water (10 ng/μL) and mixed. The DNA is incubated for 10 minutes at 50° C. 550 μL of freshly prepared DNA Modification Reagent I is added and vortexed. The mixture is incubated at 50° C. for 4-16 hours in a heat block or water bath protected from light. DNA is resuspended in DNA Modification III by vortexing vigorously. The suspension is drawn into and out of a 1 ml plastic pipette tip 10× to disperse any remaining clumps. 5 μL of well-suspended DNA Modification Reagent III is added to the DNA solutions in the tubes. 750 μL of DNA Modification Reagent II is added and mixed briefly. The mixture is incubated at room temperature for 5-10 minutes. The tubes are spun for 10 seconds at 5,000×g to pellet the DNA Reagent III. Supernatant is discarded. 1.0 mL of 70% EtOH is added, vortexed, centrifuged for 10 seconds at 5,000×g and the supernatant is discarded. This step is performed for a total of 3 times. After removing the supernatant from the third wash, the tube is centrifuged at high speed for 2 minutes, and the remaining supernatant is removed. 50 μL of the 20 mM NaOH/90% EtOH solution is added to the appropriate samples. The tube is vortexed briefly to resuspend the pellet, and incubated at room temperature for 5 minutes. The tubes are spun for 10 seconds at 5,000×g to move all contents to the tip of the tube. 1.0 mL of 90% EtOH is added and vortexed to wash the pellet. The tubes are spun again and the supernatant removed. This step is repeated one additional time. After the supernatant from the second wash is removed, the sample is centrifuged at high speed for 3 minutes. The remaining supernatant is removed and the tube allowed to dry for 10-20 minutes at room temperature. The sample is incubated for 15 minutes at 50-60° C. to elute the DNA, centrifuged at high speed for 2-3 minutes and transferred to a new tube. MSP is carried out using the CpG WIZ p16 Amplification Kit (S7800, Chemicon International, Temecula, Calif.) which enables detection of methylated vs unmethylated promoter regions within the p16 gene. Ratio's of methylated vs unmethylated DNA are determined from gel densitometric analysis of ethidium bromide stained gels of the PCR products.

The following TABLE 3-B lists compounds representative of the invention and their activity in HDAC and DNMT assays. In these assays, the following grading was used: I>10 μM, 10 μM≧II≧1 μM, 1 μM≧III≧0.1 μM, and IV<0.1 μM for IC₅₀.

TABLE 3-B

| Compound | |
|---|---|
| No. | HDAC |
| 25 | I |
| 28 | I |
| 29 | III |
| 30 | II |
| 31 | I |
| 34 | I |
| 36 | II |

TABLE 4-A

| | |
|---|---|
| | (VI) |
| 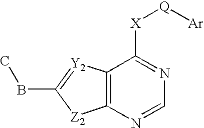 | |
| | (VII) |
| 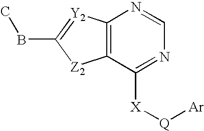 | |

SECTION 4:

| Compound # | Structure |
|---|---|
| 1 | 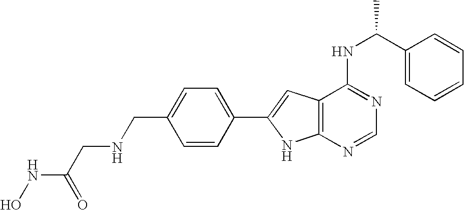 |
| 2 | 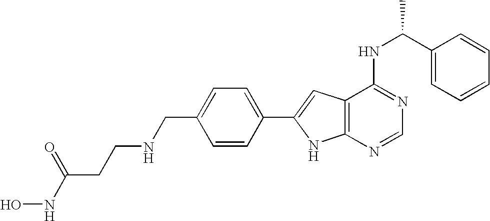 |
| 3 | 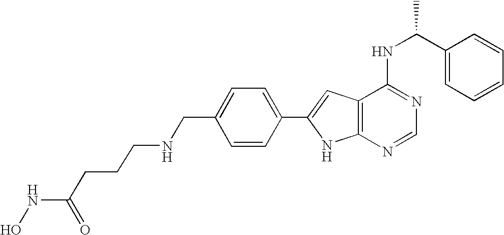 |
| 4 | 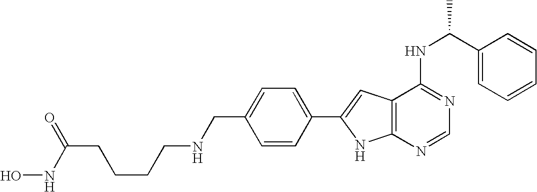 |
| 5 | 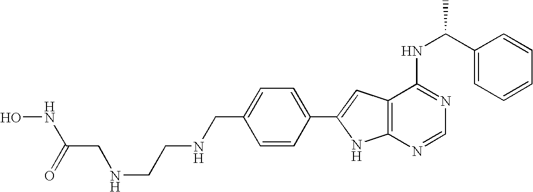 |
| 6 | 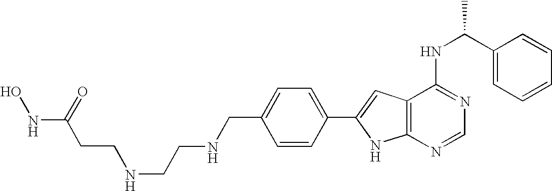 |
| 7 | 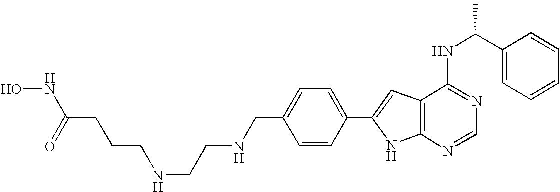 |
| 8 | 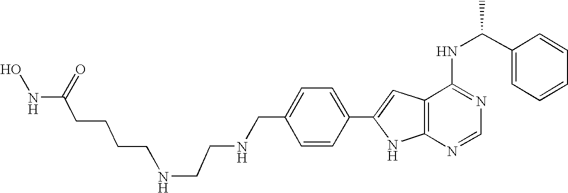 |
| 9 | 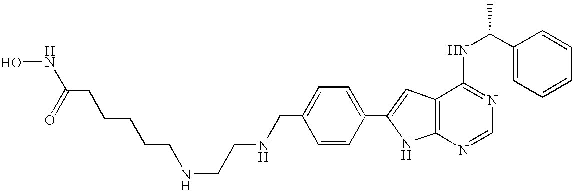 |
| 10 | 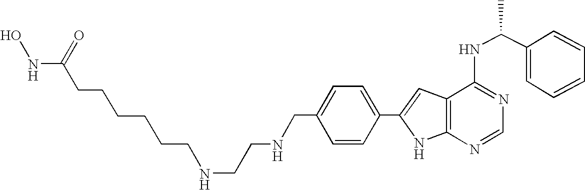 |
| 11 | 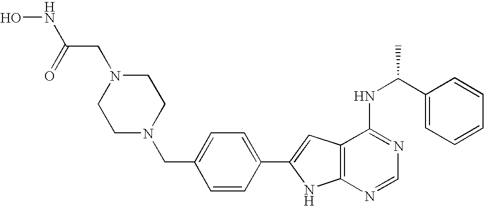 |
| 12 | 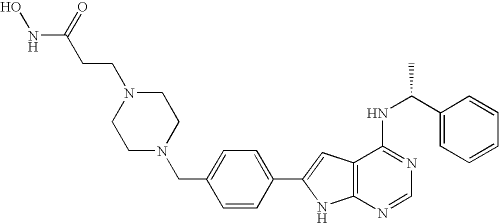 |
| 13 | 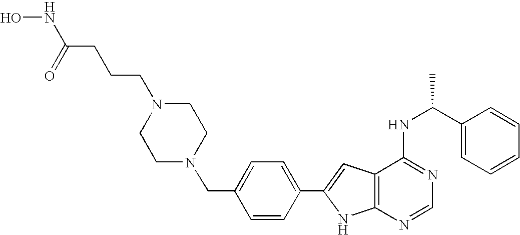 |
| 14 | 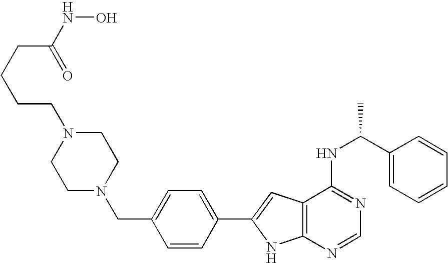 |
| 15 | 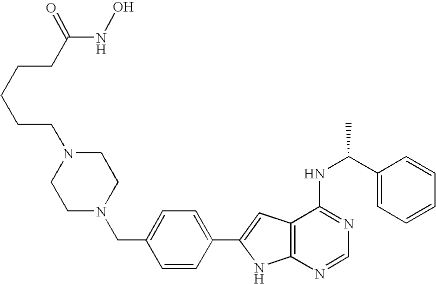 |
| 16 | 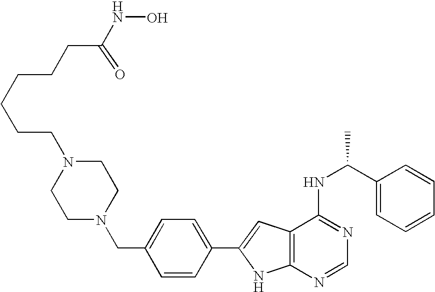 |
| 17 | 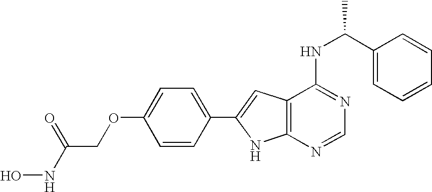 |
| 18 | 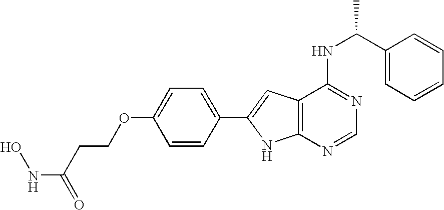 |
| 19 | 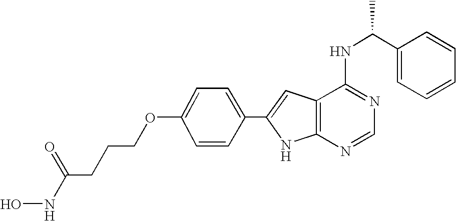 |
| 20 | 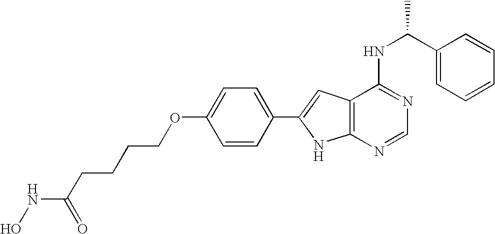 |
| 21 | 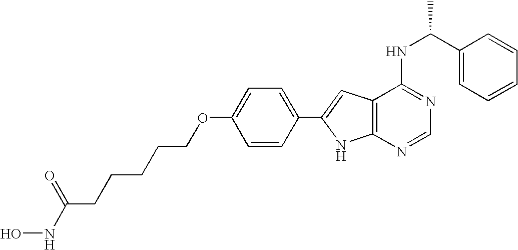 |
| 22 | 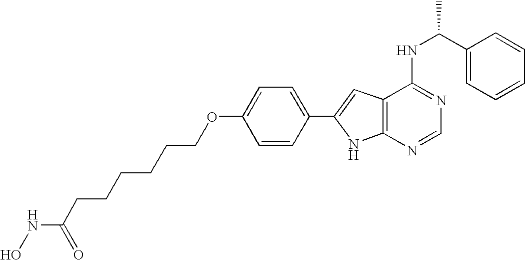 |
| 23 | 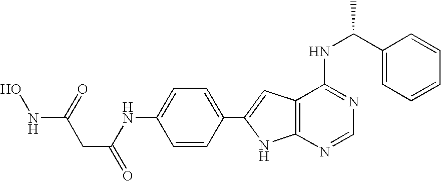 |
| 24 | 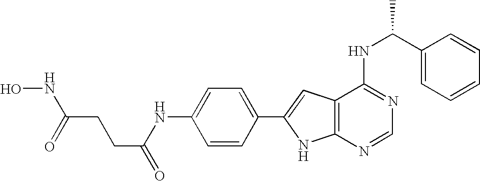 |
| 25 | 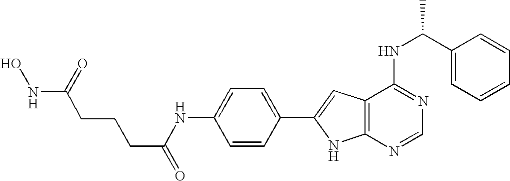 |
| 26 | 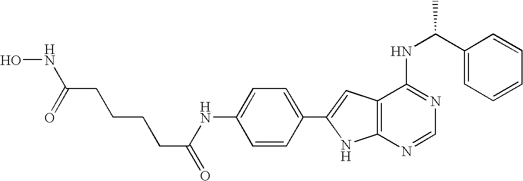 |
| 27 | 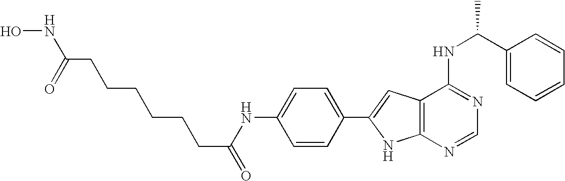 |
| 28 | 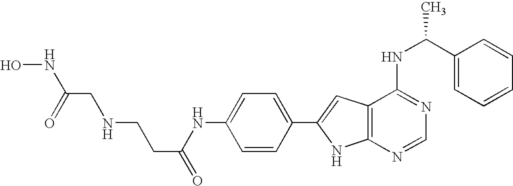 |
| 29 | 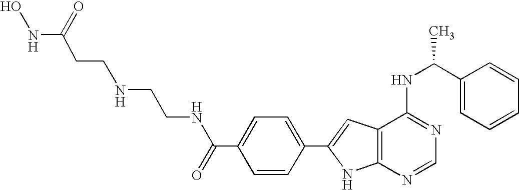 |
| 30 | 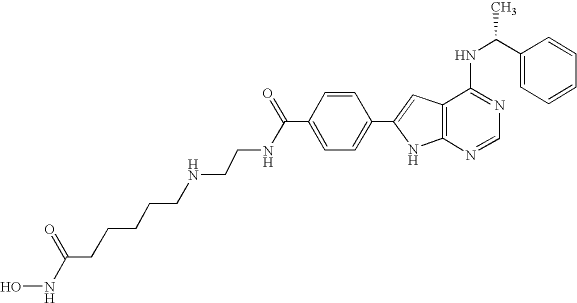 |
| 31 | 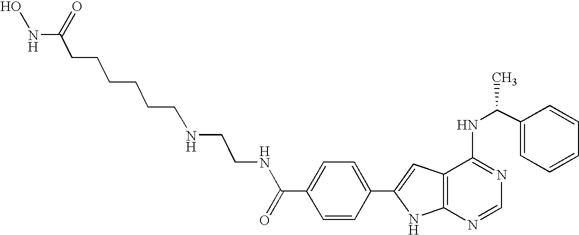 |
| 32 | 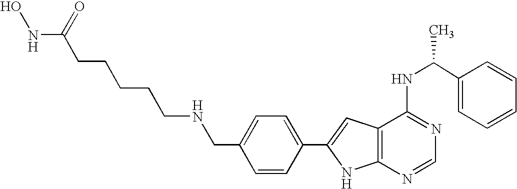 |
| 33 | 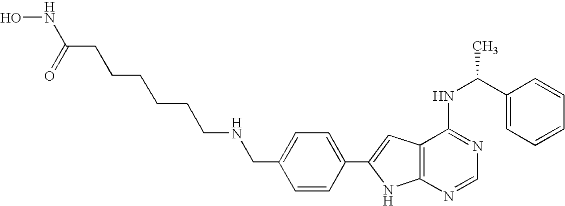 |
| 34 | 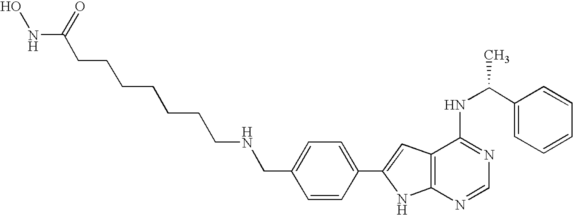 |
| 35 | 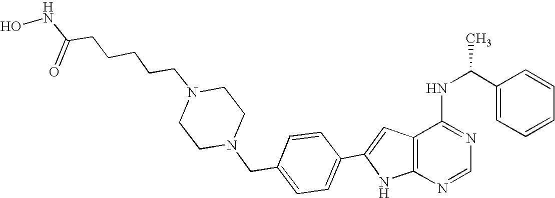 |
| 36 | 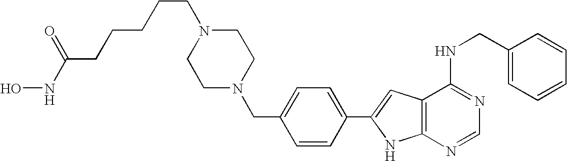 |
| 37 | 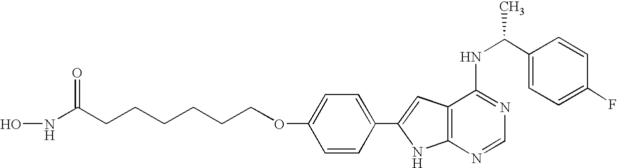 |
| 38 | 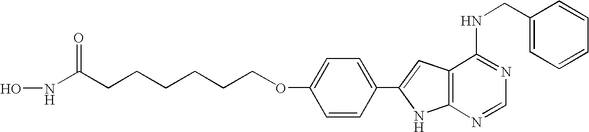 |
| 39 | 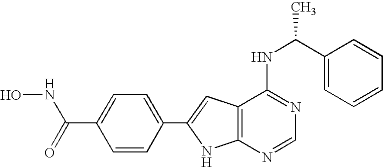 |
| 40 | 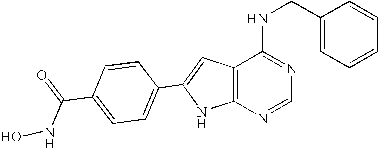 |
| 41 | 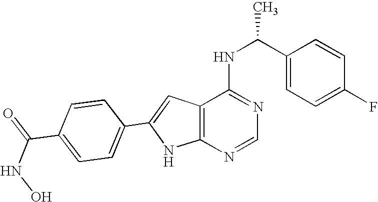 |
| 42 | 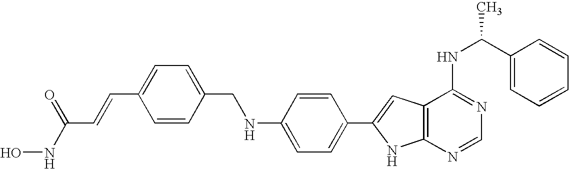 |
| 43 | 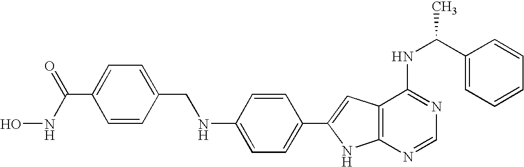 |
| 44 | 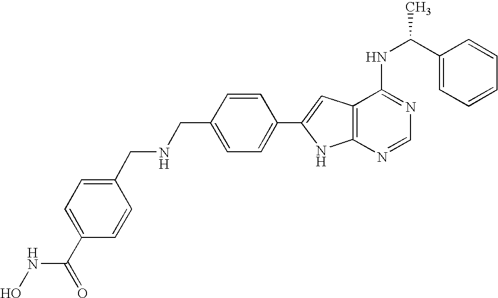 |
| 45 | 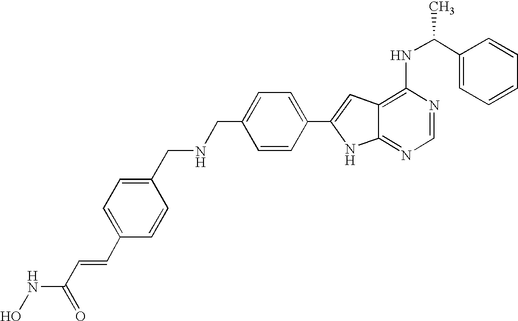 |
| 46 | 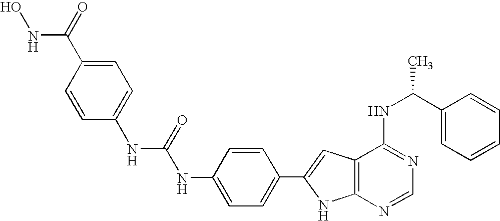 |
| 47 | 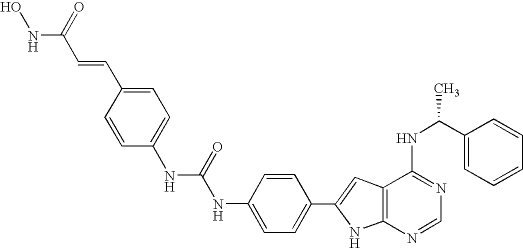 |
| 48 | 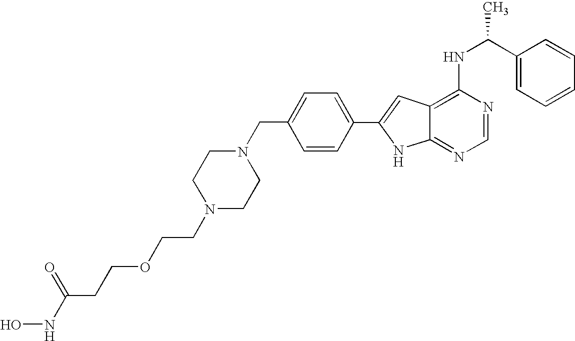 |
| 49 | 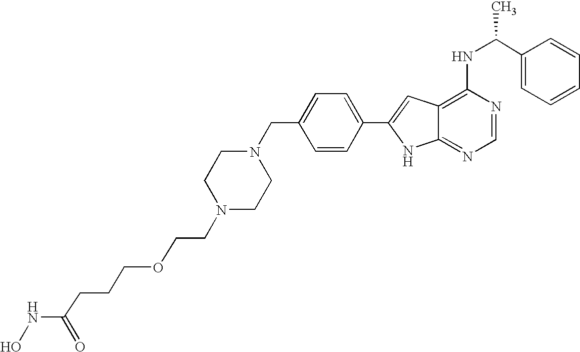 |
| 50 | 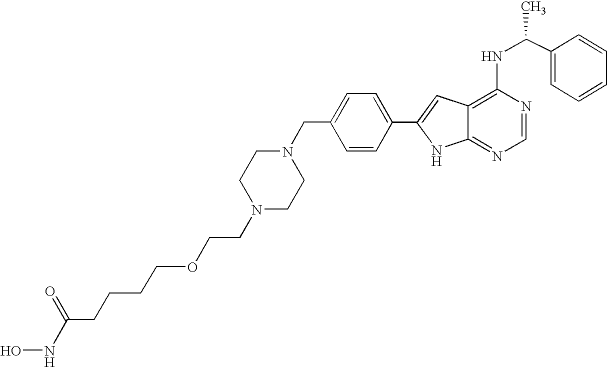 |
| 51 | 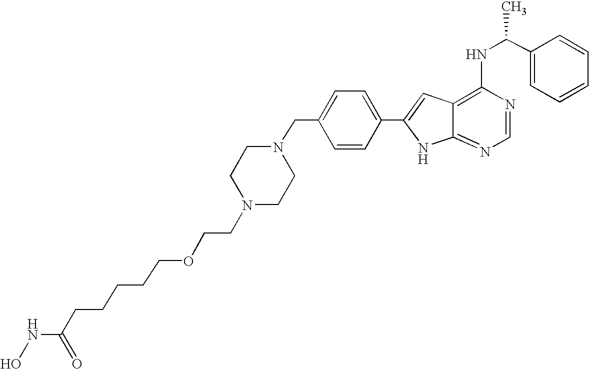 |
| 52 | 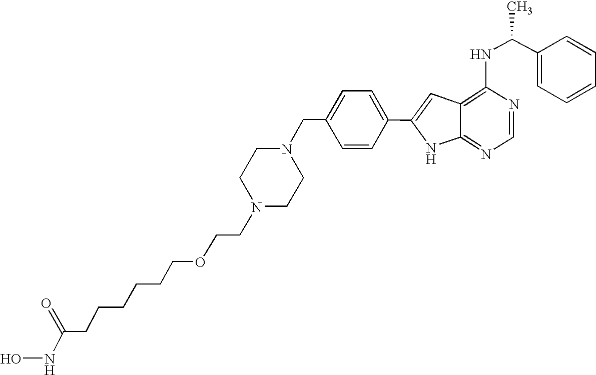 |
| 53 | 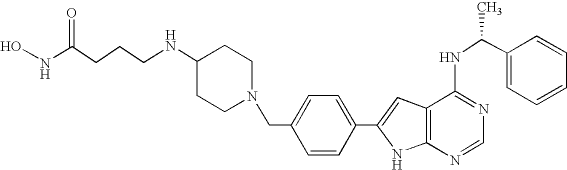 |
| 54 | 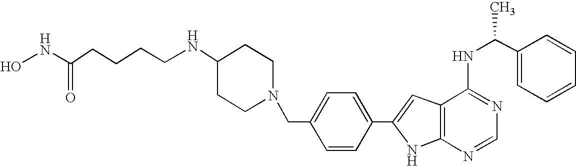 |
| 55 | 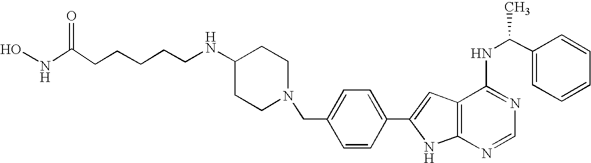 |
| 56 | 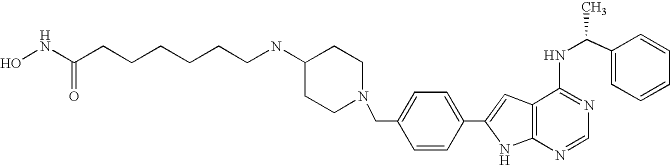 |
| 57 | 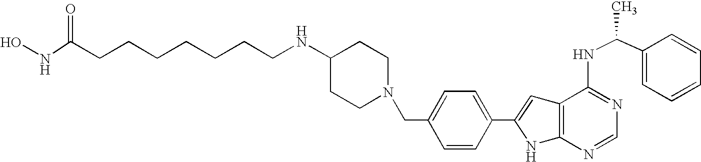 |
| 58 | 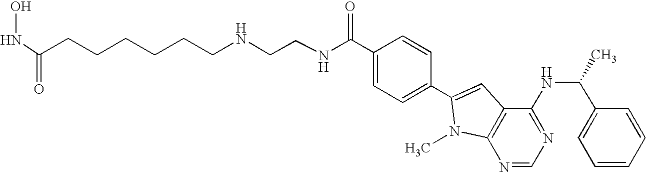 |
| 59 | 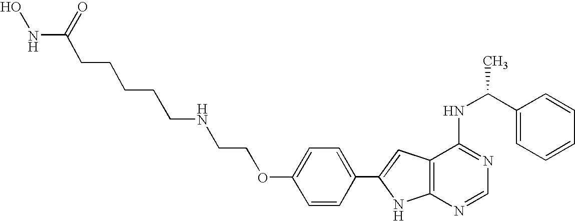 |
| 60 | 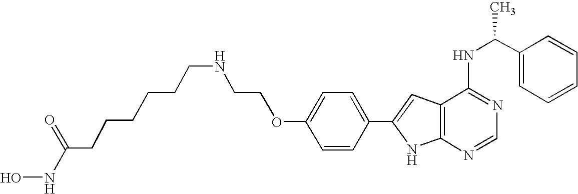 |
| 61 | 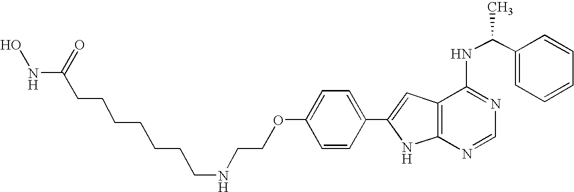 |
| 62 | 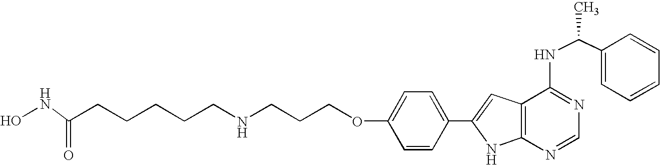 |
| 63 | 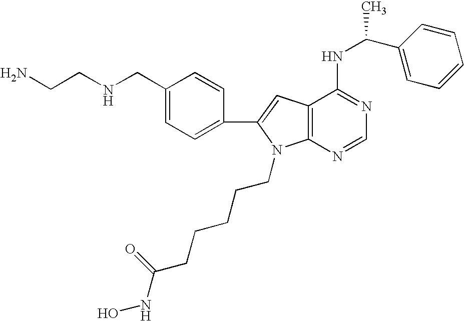 |
| 64 | 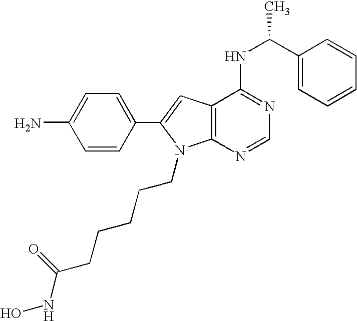 |
| 65 | 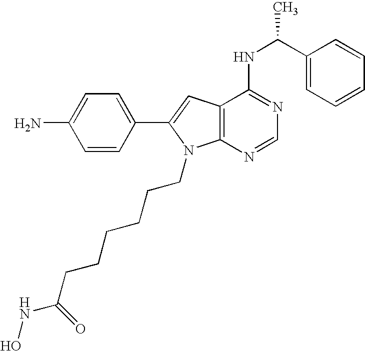 |
| 66 | 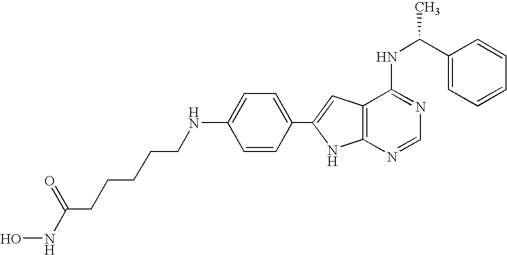 |
| 67 | 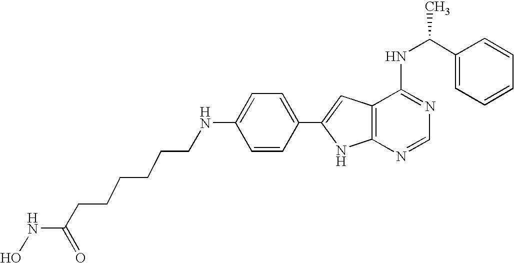 |
| 68 | 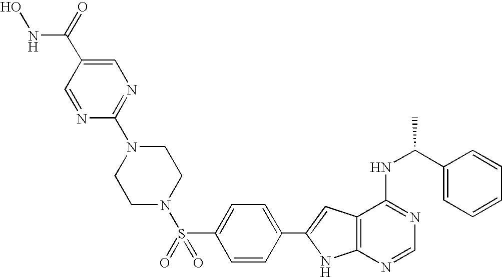 |
| 69 | 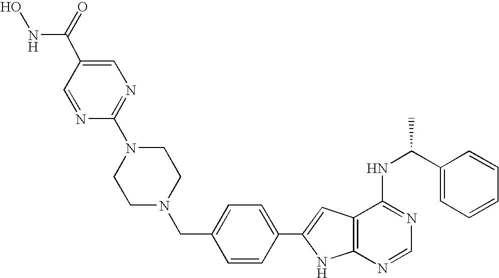 |

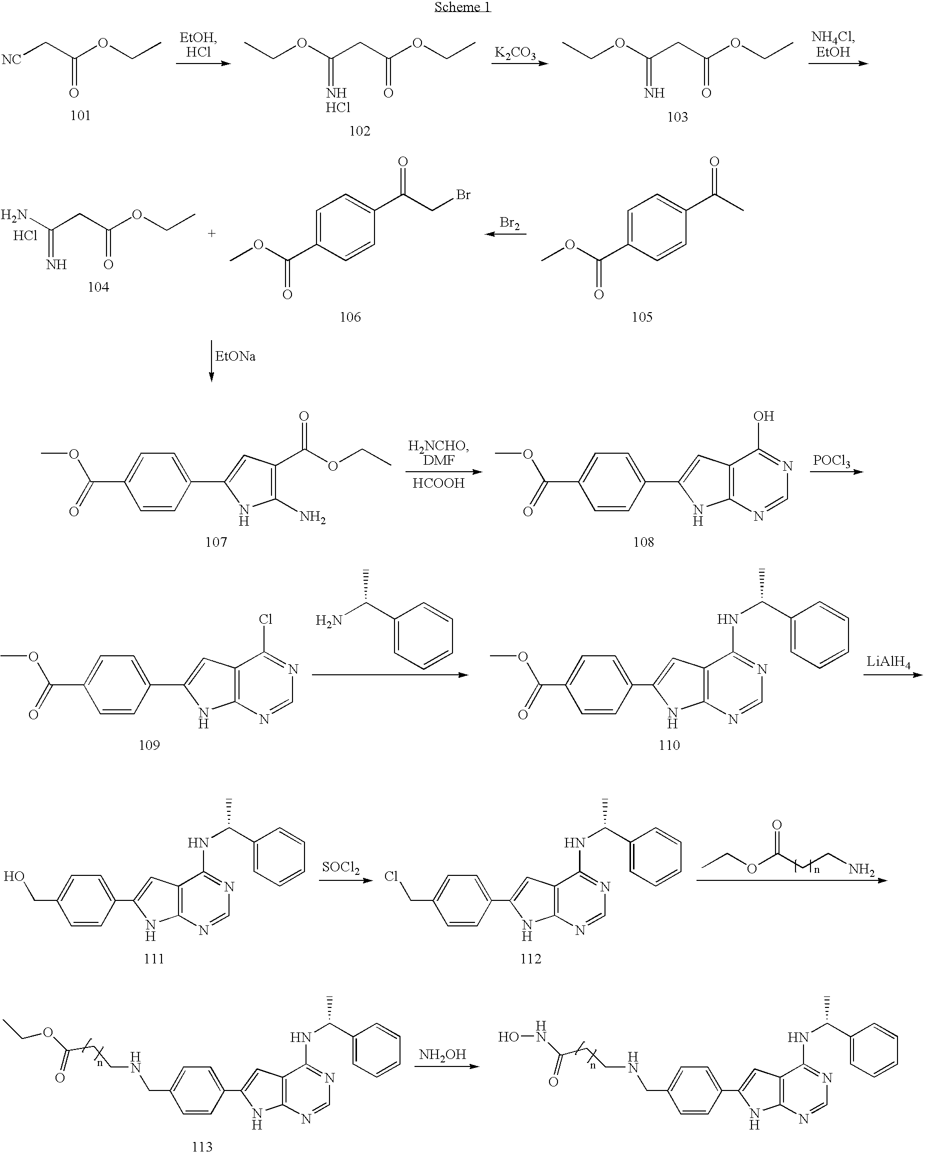

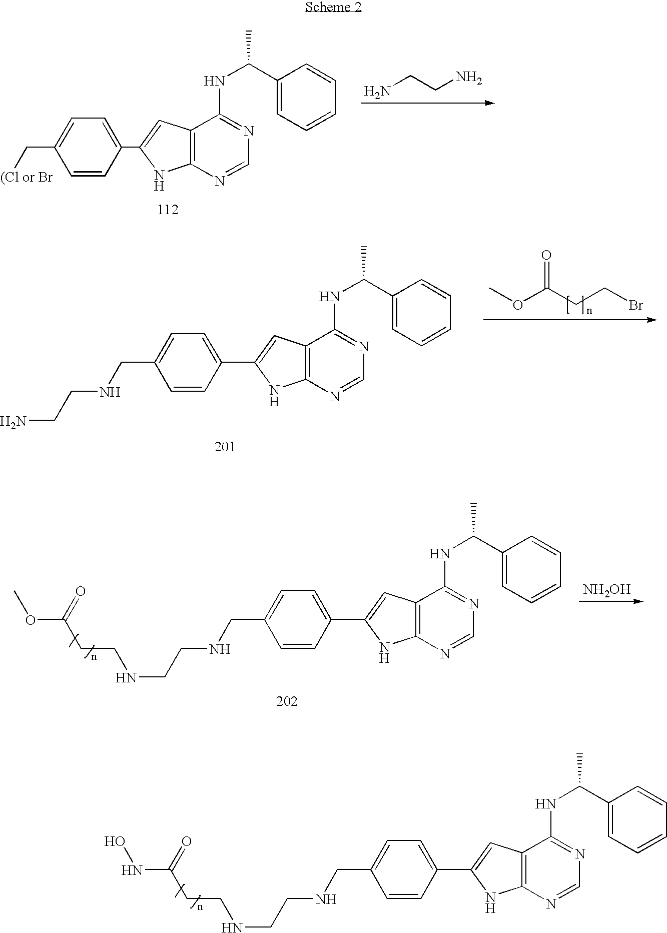

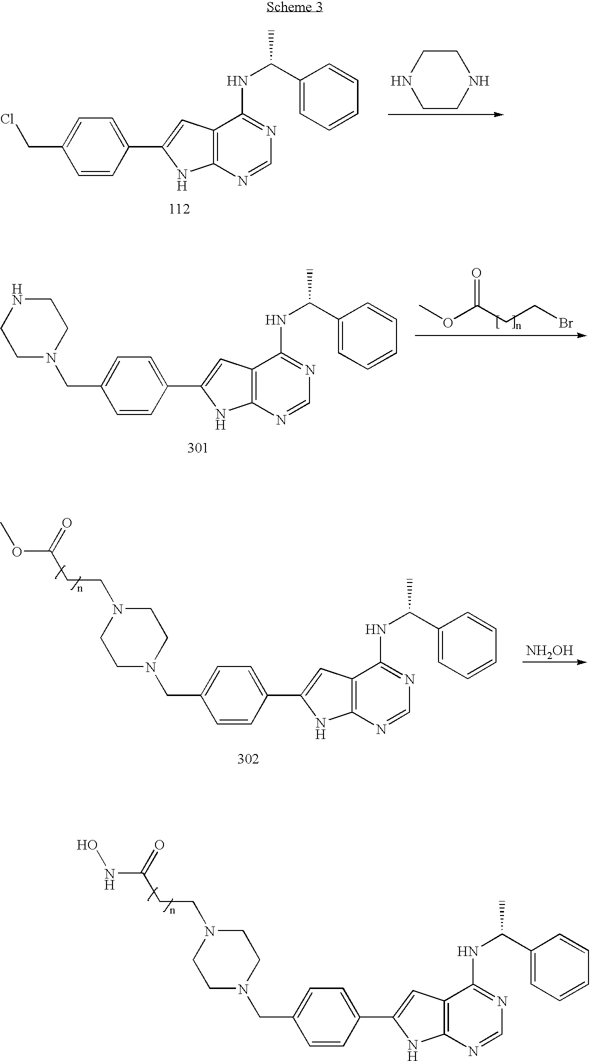

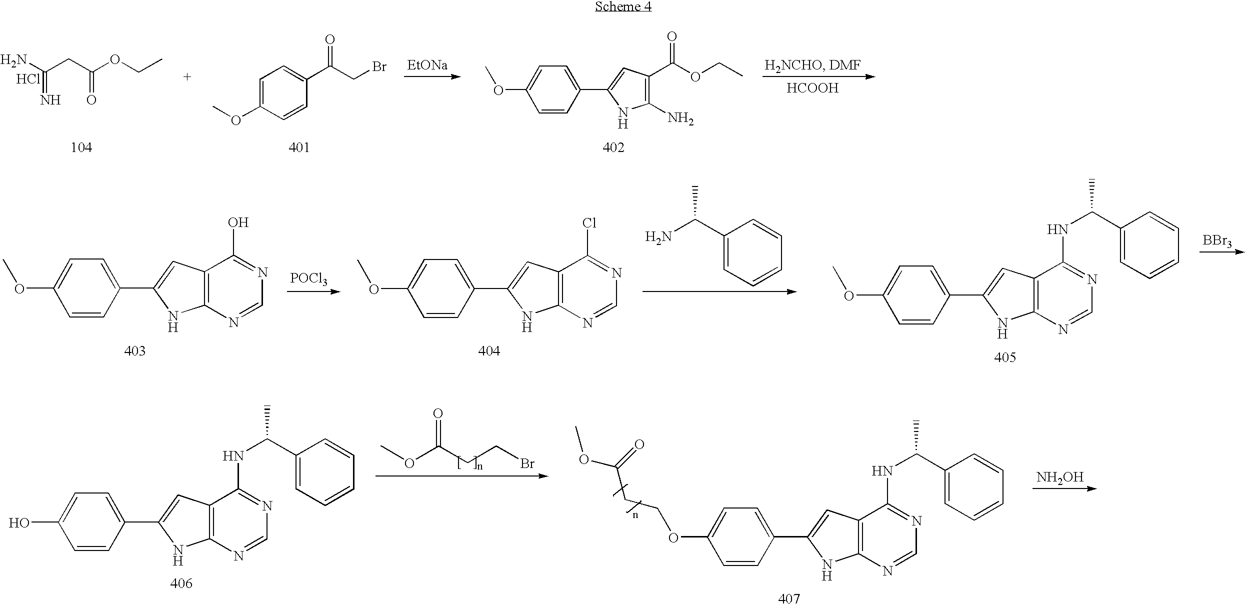

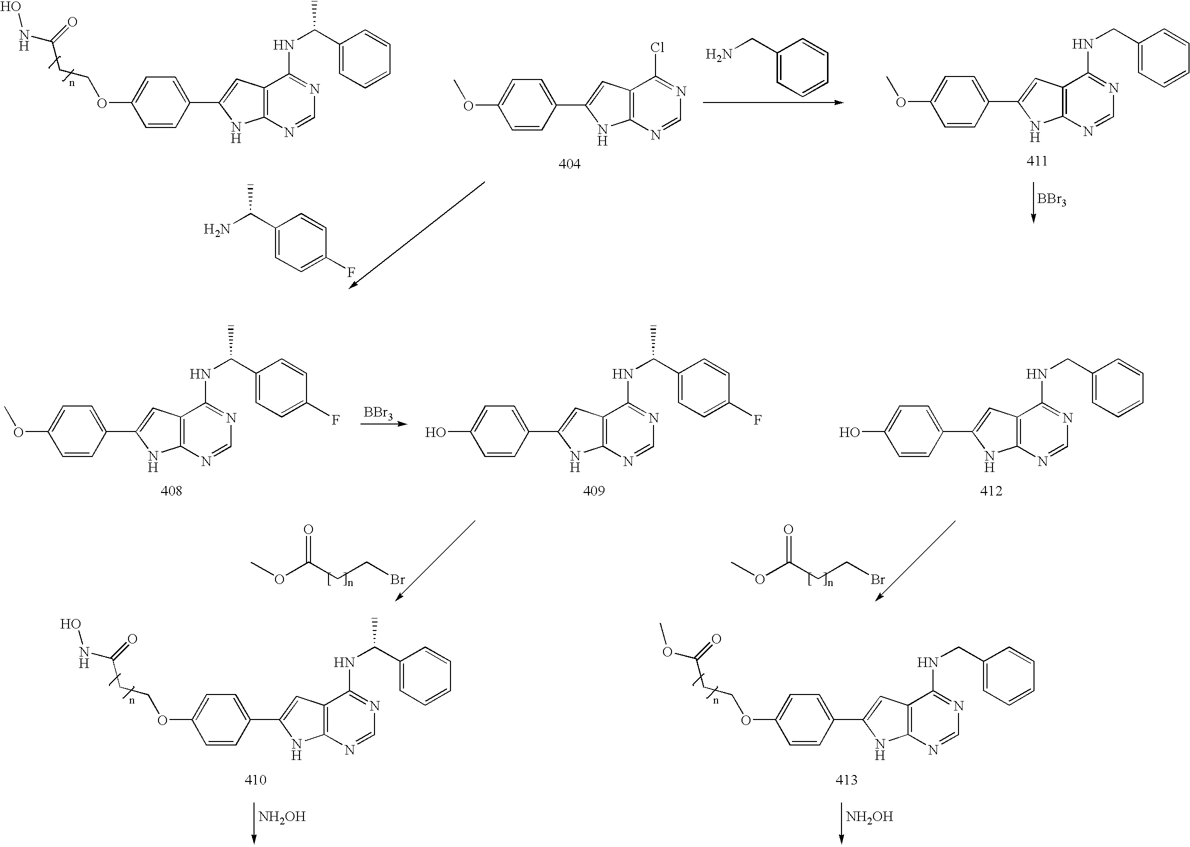

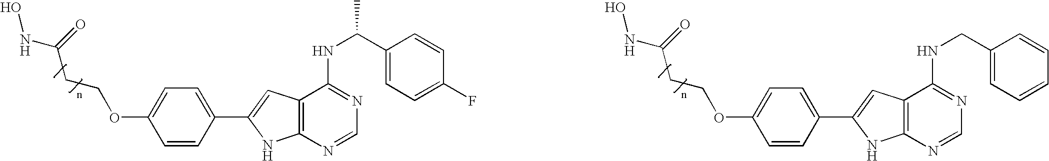

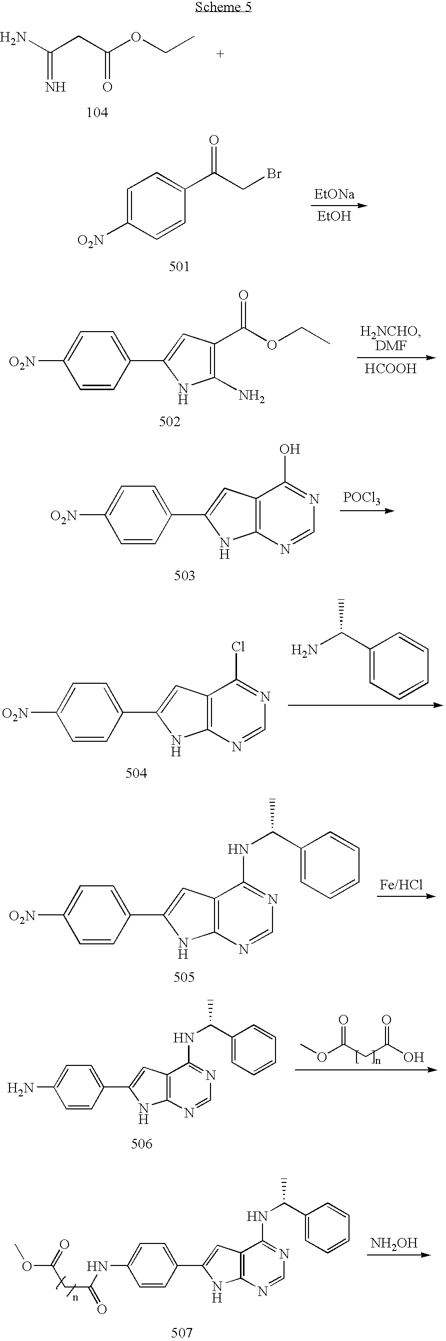

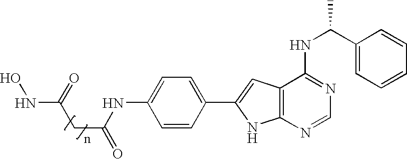

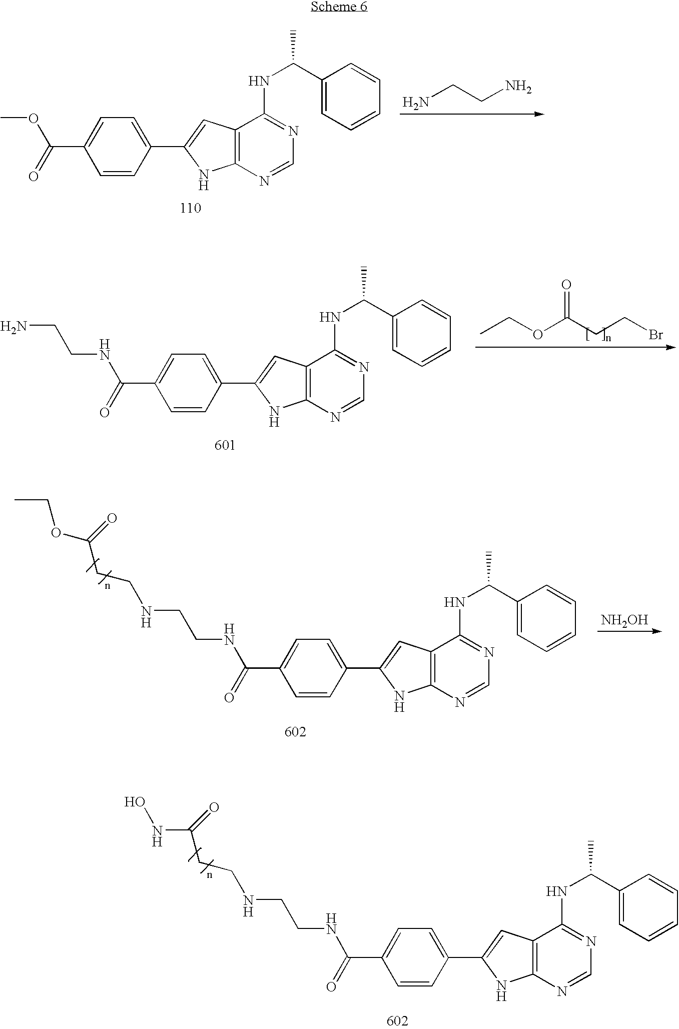

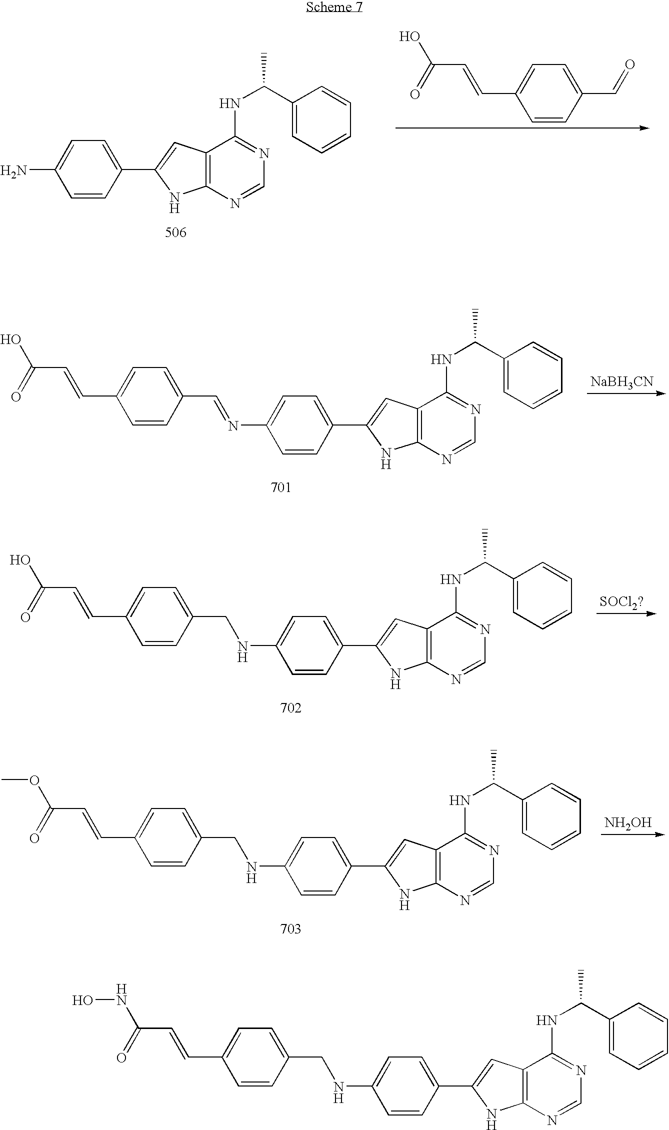

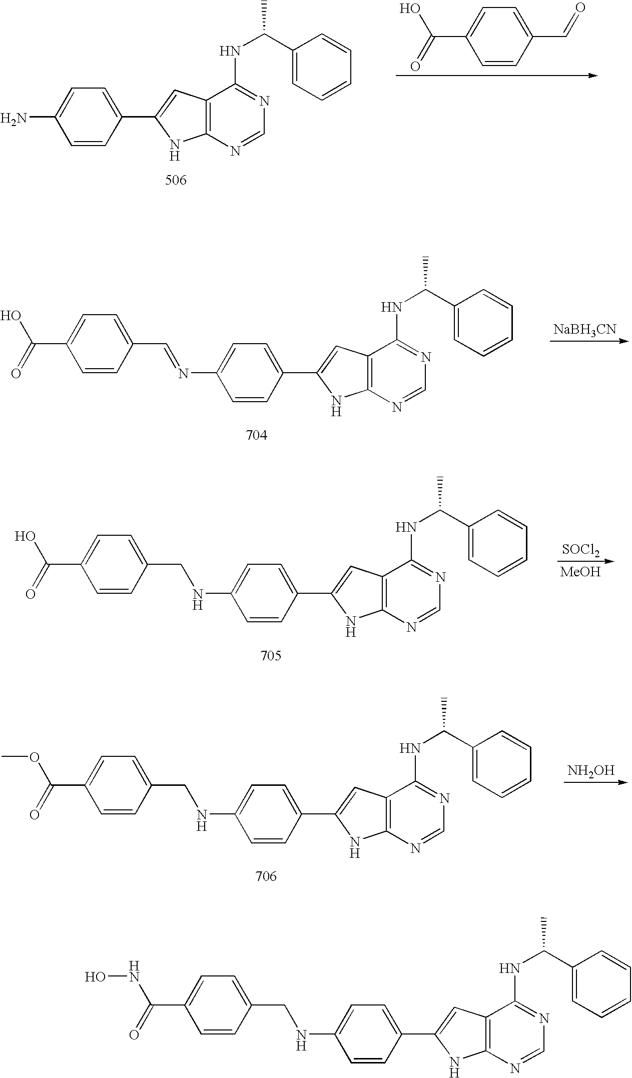

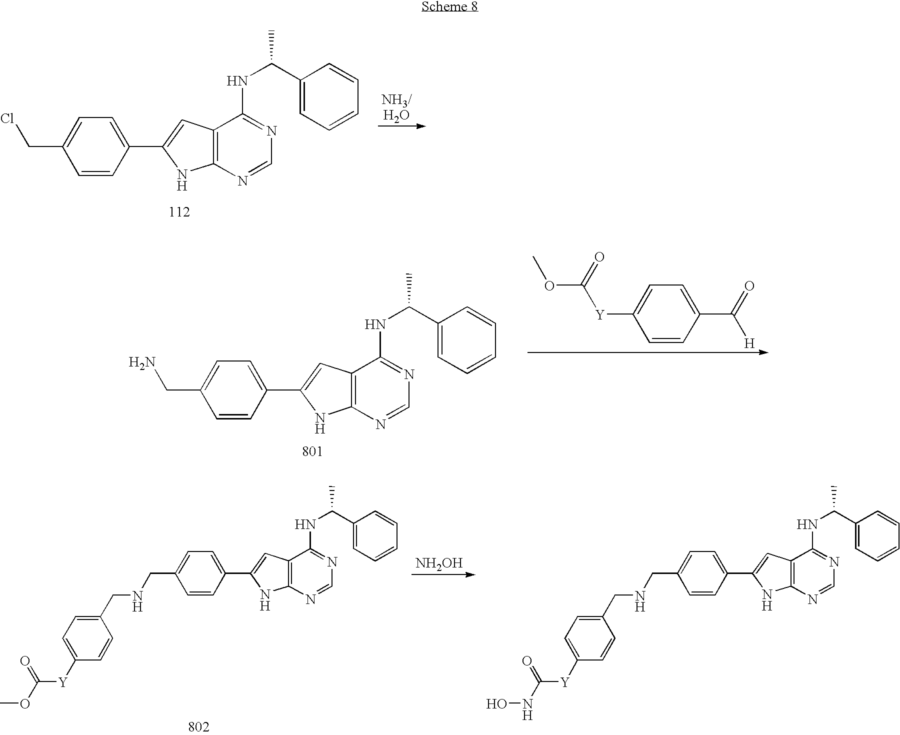

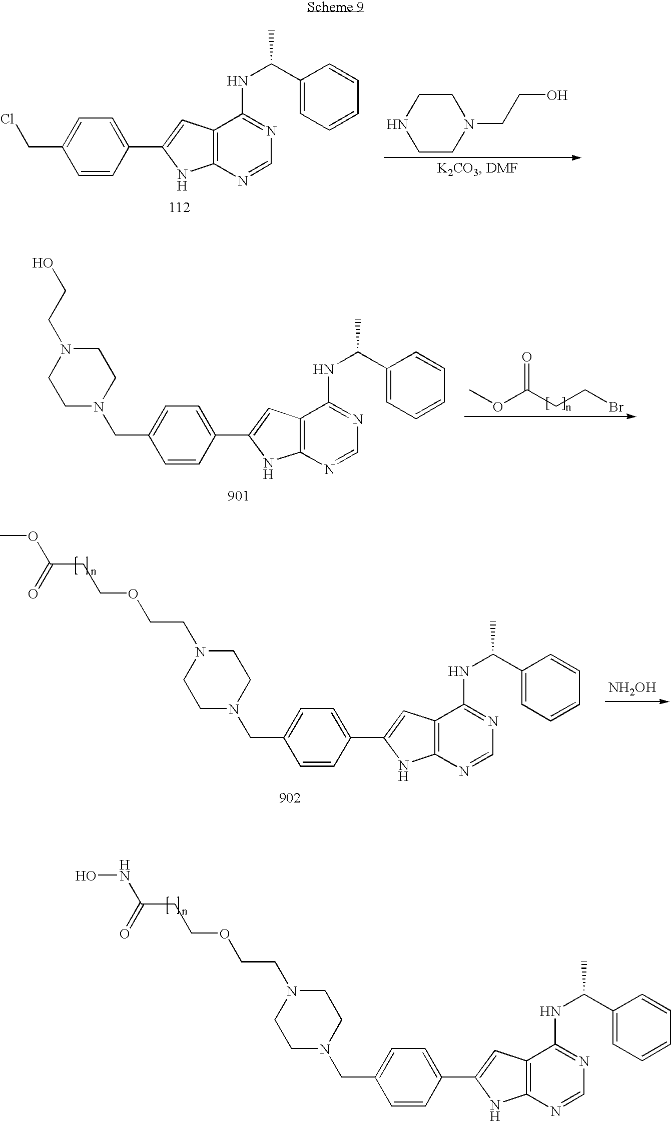

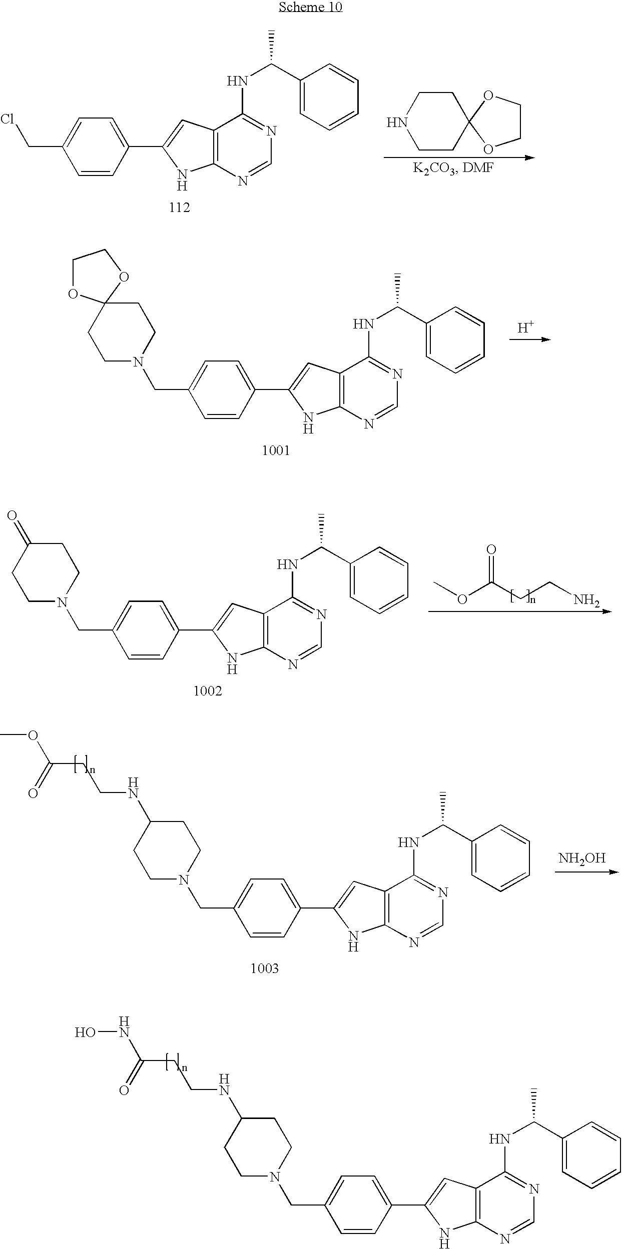

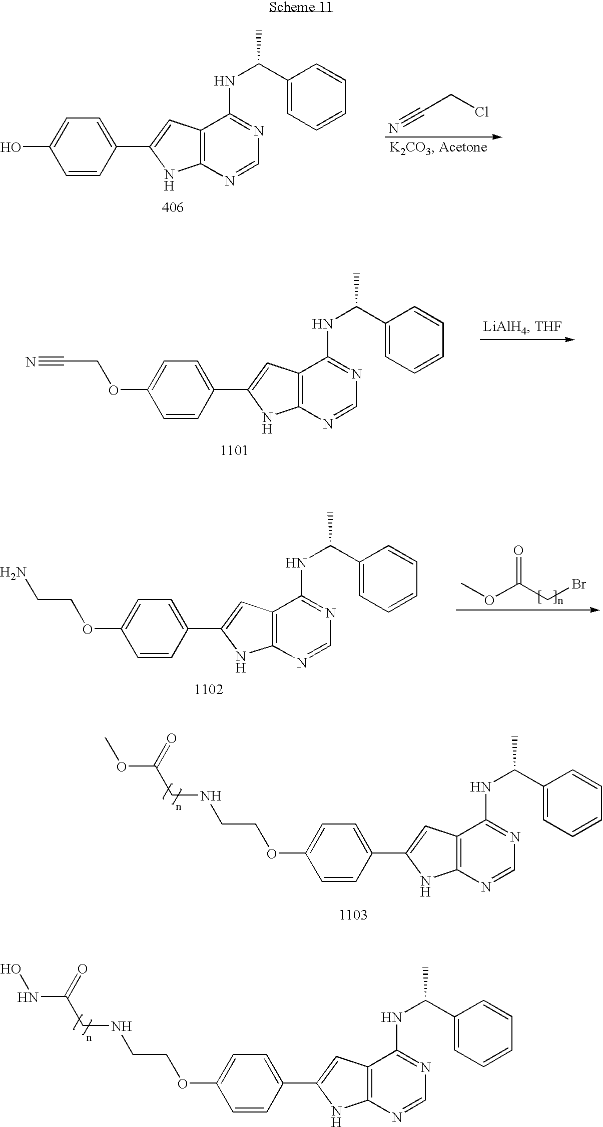

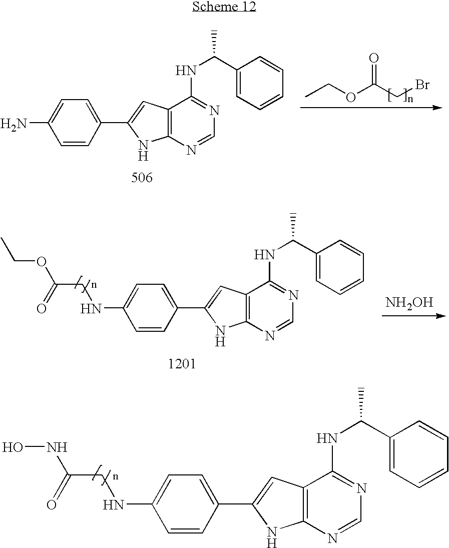

### Example 1

### Preparation of (R)—N-hydroxy-2-(4-(4-(1-phenylethylamino)-7H-pyrrolo[2,3-d]pyrimidin-6-yl)phenoxy)acetamide (Compound 17)

### Step 1a. Ethyl 2-amino-5-(4-methoxyphenyl)-1H-pyrrole-3-carboxylate (Compound 402)

To the solution of EtONa (4.08 g, 60 mmol) in EtOH (60 mL) was added compound 104 (10 g, 60 mmol) at 0° C. under nitrogen. The mixture was stirred for 20 minutes and 2-bromo-4′-methyloxy-acetophenone was added. After stirring at room temperature overnight, the mixture was concentrated and the residue was taken up in ethyl acetate, washed with water, brine, dried and concentrated to give a residue which was purified by column chromatography to afford the product 402 as a solid (5.2 g, 67% yield). ¹H NMR (DMSO-d₆) δ 10.62 (s, 1H), 7.41 (d, J=6.6 Hz, 2H), 6.88 (d, J=6.6 Hz, 2H), 6.30 (d, J=3.0 Hz, 1H), 5.59 (s, 2H), 4.13 (q, J=6.9 Hz, 2H), 3.74 (s, 3H), 1.24 (t, J=7.2 Hz, 3H). LC-MS: 260 (M+1).

### Step 1b. 6-(4-Methoxyphenyl)-7H-pyrrolo[2,3-d]pyrimidin-4-ol (Compound 403)

A mixture of compound 402 (4.7 g, 18 mmol), formamide (30 mL), formic acid (7.0 mL) and N,N-dimethylformamide (15 mL) was heated to 150° C. overnight. The mixture was cooled to room temperature and filtered, washed with i-PrOH, Et₂O successively to give the product 403 as a solid (3.7 g, 86% yield). ¹H NMR (DMSO-d₆) δ 12.22 (s, 1H), 11.81 (s, 1H), 7.84 (s, 1H), 7.76 (d, J=6.6 Hz, 2H), 6.98 (d, J=6.6 Hz, 2H), 6.29 (d, J=2.4 Hz, 1H), 3.78 (s, 3H). LC-MS: 241 (M+1).

### Step 1c. 4-Chloro-6-(4-methoxyphenyl)-7H-pyrrolo[2,3-d]pyrimidine (Compound 404)

To a flask containing compound 403 (4.0 g, 16.7 mmol) was added POCl₃ (32 mL) and the mixture was heated to reflux for 2 h. The mixture was cooled and poured into ice-water, NaOH was added to pH 7. The aqueous layer was extracted with ethyl acetate (250 mL×4). The combined organic layer was washed with brine, dried and concentrated to afford the product 404 as a yellow solid (2.2 g, 50% yield). ¹H NMR (DMSO-d₆) δ 12.93 (s, 1H), 8.55 (s, 1H), 7.98 (d, J=6.9 Hz, 2H), 7.07 (d, J=6.9 Hz, 2H), 6.98 (d, J=2.1 Hz, 1H), 3.82 (s, 3H). LC-MS: 260 (M+1).

### Step 1d. (R)-6-(4-Methoxyphenyl)-N-(1-phenylethyl)-7H-pyrrolo[2,3-d]pyrimidin-4-amine (Compound 405)

A mixture of compound 404 and (R)-(+)-alpha-methylbenzylamine (2.23 g, 2.5 equiv) was added to n-BuOH and the resulting mixture was heated to 145° C. overnight. Then another portion of (R)-(+)-alpha-methylbenzylamine (440 mg, 0.5 equiv) was added to the reaction mixture. The mixture was cooled, filtered, washed with Et₂O to afford the product 405 as a yellow solid (1.8 g, 70% yield). ¹H NMR (DMSO-d₆) δ 11.88 (s, 1H), 8.01 (s, 1H), 7.68-7.71 (m, 3H), 7.39-7.42 (m, 2H), 7.25-7.30 (m, 2H), 7.17-7.19 (m, 1H), 6.93-7.01 (m, 2H), 5.49-5.51 (m, 1H), 3.77 (s, 3H), 1.51 (d, J=6.9 Hz, 3H). LC-MS: 345 (M+1).

### Step 1e. (R)-4-(4-(1-Phenylethylamino)-7H-pyrrolo[2,3-d]pyrimidin-6-yl)phenol (Compound 406)

To a solution of compound 405 (1.13 g, 3.0 mmol) in dichloromethane (80 mL) was added dropwise the solution of BBr₃ (3.0 mL) in dichloromethane (100 mL) at 0° C. under nitrogen over 1 h. After the addition was completed, the mixture was allowed to warm to room temperature and stirred for another 5 h. Then 20 mL of water was added. The aqueous layer was extracted with ethyl acetate (100 mL×3), washed with brine, concentrated to give the product 406 as a solid (500 mg, 51% yield). ¹H NMR (DMSO-d₆) δ 13.09 (s, 1H), 9.76 (br, 1H), 8.38 (d, J=3.6 Hz, 1H), 7.68-7.73 (m, 3H), 7.55-7.57 (m, 2H), 7.43-7.48 (m, 2H), 7.34-7.39 (m, 1H), 6.94-6.96 (m, 2H), 5.49-5.50 (m, 1H), 1.73 (d, J=6.9 Hz, 3H). LC-MS: 331 (M+1).

### Step 1f. (R)-Ethyl 2-(4-(4-(1-phenylethylamino)-7H-pyrrolo[2,3-d]pyrimidin-6-yl)phenoxy)acetate (Compound 407-17)

To a mixture of compound 406 (100 mg, 0.3 mmol) and K₂CO₃ (70 mg, 0.5 mmol) in dimethylformamide (1.0 mL) was added ethyl 2-bromoacetate (50 mg, 0.3 mmol) and the mixture was stirred at room temperature for 20 h. 5 ml of water was added and the mixture was extracted with ethyl acetate (25 mL×4), dried and concentrated to give a residue which was purified by column chromatography to afford the product 407-17 as a white solid (40 mg, 32% yield). ¹H NMR (DMSO-d₆) δ 11.89 (s, 1H), 8.01 (s, 1H), 7.67-7.72 (m, 3H), 7.39-7.42 (d, J=8.1 Hz, 2H), 7.25-7.31 (m, 2H), 7.17-7.20 (m, 1H), 6.94-7.00 (m, 2H), 5.46-5.48 (m, 1H), 4.80 (s, 2H), 4.16 (q, J=6.9 Hz, 2H), 1.51 (d, J=6.9 Hz, 3H), 1.20 (t, J=7.2 Hz, 3H). LC-MS: 417 (M+1).

### Step 1g. (R)—N-Hydroxy-2-(4-(4-(1-phenylethylamino)-7H-pyrrolo[2,3-d]pyrimidin-6-yl)phenoxy)acetamide (Compound 17)

Preparation of the solution of hydroxylamine in methanol: hydroxylamine hydrochloride (4.67 g, 67 mmol) was dissolved in methanol (24 mL) made to solution A. Potassium hydroxide (5.61 g, 100 mmol) was dissolved in methanol (14 mL) made to solution B. The solution A was cooled to 0° C., and solution B was added into solution A dropwise. The mixture was stirred for 30 minutes at 0° C., and was allowed to stand at low temperature for some time. The precipitate was isolated to afford the solution of hydroxylamine in methanol.

To a flask containing compound 407-17 (35 mg, 0.084 mmol) was added the above solution of hydroxylamine in methanol (2.0 mL). The mixture was stirred at room temperature for 30 min. Then it was adjusted to PH 7 using concentrated HCl. The mixture was concentrated to give a residue which was purified by column chromatography to afford the product 17 as a solid (25 mg, 71% yield). ¹H NMR (DMSO-d₆) δ 11.91 (s, 1H), 8.03 (s, 1H), 7.69-7.73 (m, 3H), 7.41-7.44 (m, 2H), 7.27-7.32 (m, 2H), 7.19-7.21 (m, 1H), 6.96-7.04 (m, 2H), 6.48-6.50 (m, 1H), 4.50 (s, 2H), 1.53 (d, J=6.9 Hz, 3H). LC-MS: 404 (M+1). Mp: 116.8-126.8.

### Example 2

### (R)—N-hydroxy-6-(4-(4-(1-phenylethylamino)-7H-pyrrolo[2,3-d]pyrimidin-6-yl)phenoxy)hexanamide (Compound 21)

### Step 2a. (R)-Ethyl-6-(4-(4-(1-phenylethylamino)-7H-pyrrolo[2,3-d]pyrimidin-6-yl)phenoxy)hexanoate (Compound 407-21)

To a mixture of compound 406 (330 mg, 1.0 mmol) and K₂CO₃ (210 mg, 1.5 mmol) in dimethylformamide (2.0 mL) was added ethyl 6-bromohexanoate (223 mg, 1.0 mmol) and the mixture was stirred at 40° C. for 20 hours. 5 ml of water was added and the mixture was extracted with ethyl acetate (25 mL×4), dried and concentrated to give a residue which was purified by column chromatography to afford the product 407-21 as a white solid (250 mg, 53% yield). ¹H NMR (DMSO-d₆) δ 11.87 (s, 1H), 8.01 (s, 1H), 7.66-7.69 (m, 3H), 7.39-7.42 (m, 2H), 7.25-7.30 (m, 2H), 7.17-7.19 (m, 1H), 6.92-6.99 (m, 2H). 5.46-5.48 (m, 1H), 3.95-4.07 (m, 4H), 2.29 (t, J=7.2 Hz, 2H), 1.68-1.73 (m, 2H), 1.38-1.60 (m, 8H), 1.15 (t, J=7.2 Hz, 3H). LC-MS: 473 (M+1).

### Step 2b. (R)—N-Hydroxy-6-(4-(4-(1-phenylethylamino)-7H-pyrrolo[2,3-d]pyrimidin-6-yl)phenoxy)hexanamide (Compound 21)

Preparation of the solution of hydroxylamine in methanol: hydroxylamine hydrochloride (4.67 g, 67 mmol) was dissolved in methanol (24 mL) made to solution A. Potassium hydroxide (5.61 g, 100 mmol) was dissolved in methanol (14 mL) made to solution B. The solution A was cooled to 0° C., and solution B was added into solution A with dropwise. The mixture was stirred for 30 minutes at 0° C., and was allowed to stand at low temperature for some time. The precipitate was isolated to afford the solution of hydroxylamine in methanol.

To a flask containing compound 407-21 (220 mg, 0.466 mmol) was added above solution of hydroxylamine in methanol (3.0 mL). The mixture was stirred at room temperature for 2 h. Then it was adjusted to PH 7 using concentrated HCl. The mixture was concentrated to give a residue which was purified by column chromatography to afford the product 21 as a white solid (130 mg, 61% yield). ¹H NMR (DMSO-d₆) δ 11.87 (s, 1H), 10.32 (s, 1H), 8.64 (s, 1H), 8.00 (s, 1H), 7.66-7.69 (m, 3H), 7.39-7.41 (m, 2H), 7.25-7.30 (m, 2H), 7.16-7.19 (m, 1H), 6.92-6.99 (m, 2H). 5.46-5.48 (m, 1H), 3.97 (t, J=6.6 Hz, 2H), 1.95 (t, J=7.2 Hz, 2H), 1.67-1.72 (m, 2H), 1.20-1.39 (m, 8H). LC-MS: 460 (M+1).

### Example 3

### (R)—N-hydroxy-7-(4-(4-(1-Phenylethylamino)-7H-pyrrolo[2,3-d]pyrimidin-6-yl)phenoxy)heptanamide (Compound 22)

### Step 3a. (R)-Ethyl-7-(4-(4-(1-phenylethylamino)-7H-pyrrolo[2,3-d]pyrimidin-6-yl)phenoxy)heptanoate (Compound 407-22)

To a mixture of compound 406 (330 mg, 1.0 mmol) and K₂CO₃ (210 mg, 1.5 mmol) in dimethylformamide (2.0 mL) was added ethyl 7-bromoheptanoate (237 mg, 1.0 mmol) and the mixture was stirred at 40° C. for 20 h. 5 ml of water was added and the mixture was extracted with ethyl acetate (25 mL×4), dried and concentrated to give a residue which was purified by column chromatography to afford the product 407-22 as a white solid (150 mg, 31% yield). ¹H NMR (DMSO-d₆) δ 11.87 (s, 1H), 8.01 (s, 1H), 7.66-7.69 (m, 3H), 7.41 (d, J=7.5 Hz, 2H), 7.25-7.30 (m, 2H), 7.17-7.19 (m, 1H), 6.92-6.99 (m, 2H), 5.46-5.48 (m, 1H), 3.95-4.06 (m, 4H), 2.24-2.29 (t, J=7.2 Hz, 2H), 1.67-1.71 (m, 2H), 1.31-1.55 (m, 10H), 1.15 (t, J=7.2 Hz, 3H). LC-MS: 487 (M+1).

### Step 3b. (R)—N-Hydroxy-7-(4-(4-(1-phenylethylamino)-7H-pyrrolo[2,3-d]pyrimidin-6-yl)phenoxy)heptanamide (Compound 22)

Preparation of the solution of hydroxylamine in methanol: hydroxylamine hydrochloride (4.67 g, 67 mmol) was dissolved in methanol (24 mL) made to solution A. Potassium hydroxide (5.61 g, 100 mmol) was dissolved in methanol (14 mL) made to solution B. The solution A was cooled to 0° C., and solution B was added into solution A dropwise. The mixture was stirred for 30 minutes at 0° C., and was allowed to stand time at low temperature for some time. The precipitate was isolated to afford the solution of hydroxylamine in methanol.

To a flask containing compound 407-22 (120 mg, 0.247 mmol) was added above solution of hydroxylamine in methanol (3.0 mL). The mixture was stirred at room temperature for 2 h. Then it was adjusted to pH 7 using concentrated HCl. The mixture was concentrated to give a residue which was purified by column chromatography to afford the product 22 as a white solid (90 mg, 77% yield). ¹H NMR (DMSO-d₆) δ 11.87 (s, 1H), 10.30 (s, 1H), 8.62 (s, 1H), 8.00 (s, 1H), 7.66-7.69 (m, 3H), 7.39-7.42 (m, 2H), 7.25-7.30 (m, 2H), 7.16-7.19 (m, 1H), 6.91-6.99 (m, 2H). 5.48-5.49 (m, 1H), 3.97 (t, J=6.6 Hz, 2H), 1.93 (t, J=6.9 Hz, 2H), 1.67-1.72 (m, 2H), 1.20-1.51 (m, 10H). LC-MS: 474 (M+1).

### Example 4

### (R)—N-Hydroxy-2-(4-(4-(1-phenylethylamino)-7H-pyrrolo[2,3-d]pyrimidin-6-yl)benzylamino)acetamide (Compound 1)

### Step 4a. Ethyl 3-amino-3-iminopropanoate hydrochloride (Compound 104)

To anhydrous ethanol (460 g, 10.0 mol) at −30° C. was bubbled in anhydrous hydrogen chloride until the total weight of 821 g of HCl/EtOH solution (44% (w/w) was obtained.

Ethyl cyanoacetate (452 g) was added into the HCl/EtOH solution (292 g), the mixture was cooled to ice-salt bath temperature and stirred for 1 hours. The reaction was warmed to room temperature and stood overnight. A white precipitate of 102 was obtained and this mixture was used directly in the next step.

The obtained mixture was added to a mixture of ether and a solution of K₂CO₃ (828 g) in water (2500 mL). The ether layer was separated, dried over Na₂SO₄, and filtered. The filtrate was concentrated under reduced pressure to give compound 103 (445 g) as a colorless oil.

A mixture of compound 103 (445 g) and ammonium chloride (149.5 g) in ethanol (1500 mL) was heated to reflux for 8 h. The solid was isolated and the filtrate was concentrated. The residue was washed with ether and acetone to give product 104 (220 g, 33% total yield in three steps). LCMS: 131 [M+1]⁺, ¹H NMR (DMSO-d₆): δ 1.22 (t, J=6.9 Hz, 3H), 3.68 (s, 2H), 4.16 (q, J=6.9 Hz, 2H), 9.04 (s, 2H), 9.32 (s, 2H).

### Step 4b. Methyl 4-(2-bromoacetyl)benzoate (compound 106)

Methyl 4-acetylbenzoate 105 (8.91 g, 50 mmol) was suspended in acetic acid (80 mL) and the mixture was stirred until a clear solution was reached. Then bromine (8.39 g, 52 mmol) was added dropwise to the mixture. The mixture was stirred at room temperature until the strong orange color was disappeared. The solution was cooled to 0° C. and the solid was collected and washed with 50% aqueous methanol, dried to give the title compound 106 (9.9 g, 77%): LCMS: 257 [M+1]⁺; ¹H NMR (CDCl₃): δ: 3.96 (s, 3H), 4.47 (s, 2H), 8.03 (t, 1H), 8.06 (t, 1H), 8.14 (t, 1H), 8.16 (t, 1H).

### Step 4c. Ethyl 5-(4-(methoxycarbonyl)phenyl)-2-amino-1H-pyrrole-3-carboxylate (Compound 107)

Sodium (1.38 g, 60 mmol) was added to ethanol (150 mL) and stirred until the sodium was dissolved. The reaction was cooled to 0° C. and a solution of ethyl 2-amidinoacetate hydrochloride (10.0 g, 0.06 mol) was added and stirred for 30 min. Methyl 4-(2-bromoacetyl)benzoate 106 (7.71 g, 0.03 mol) was then added. The resulting mixture was stirred at room temperature for 24 h. The reaction mixture was concentrated and the residue was dissolved with ethyl acetate, filtered and the filtrate was washed with water. The aqueous phase was extracted with ethyl acetate. The combined organic layer was washed with brine, dried over MgSO₄ and filtered. The filtrate was concentrated and the residue was purified by column chromatography to give the compound 107 (7.38 g, 85.3%). LCMS: 289 [M+1]⁻; ¹H NMR (DMSO-d₆): δ 1.25 (t, J=6.9 Hz, 3H), 3.82 (s, 3H), 4.14 (q, J=6.9 Hz, 2H), 5.81 (s, 2H), 6.71 (s, 1H), 7.61 (d, J=8.7 Hz, 2H), 7.84 (d, J=8.7 Hz, 2H), 10.94 (s, 1H).

### Step 4d. Methyl 4-(4-hydroxy-7H-pyrrolo[2,3-d]pyrimidin-6-yl)benzoate (Compound 108)

A mixture of 107 (7.0 g, 24.3 mmol), formic acid (12 mL) and formamide (50 mL) in DMF (24 mL) was heated at 150° C. for 16 hours. The reaction mixture was cooled and diluted with isopropanol and the precipitate was isolated, washed with isopropanol and hexane to give the title compound 108 (4.1 g, 62.7%). LCMS: 270 [M+1]⁻; ¹H NMR (DMSO-d₆): δ 2.30 (s, 3H), 6.84 (s, 1H), 7.19 (d, J=8.1 Hz, 2H), 7.70 (d, J=8.1 Hz, 2H), 7.84 (s, 1H), 11.80 (s, 1H), 12.24 (s, 1H).

### Step 4e. Methyl 4-(4-chloro-7H-pyrrolo[2,3-d]pyrimidin-6-yl)benzoate (Compound 109)

A mixture of compound 108 (4.1 g, 15.2 mmol) and phosphoryl trichloride (30 mL) was heated at reflux for 3 hours. The excessive phosphoryl trichloride was removed under reduced pressure. The residue was dissolved in ethyl acetate and the organic layer was washed with aqueous NaHCO₃ solution, brine, dried over MgSO₄, filtered and evaporated to give crude product 109 (5.27 g): LCMS: 288 [M+1]⁺; ¹H NMR (DMSO-d₆): δ 2.34 (s, 3H), 7.02 (s, 1H), 7.31 (d, J=8.1 Hz, 2H), 7.88 (d, J=8.1 Hz, 2H), 8.55 (s, 1H), 12.94 (s, 1H).

### Step 4f. Methyl 4-(4-((R)-1-phenylethylamino)-7H-pyrrolo[2,3-d]pyrimidin-6-yl)benzoate (Compound 110)

To a suspension of compound 109 (8.4 g, 29.0 mmol) in n-butanol (100 ml) was added (R)-phenethylamine (4.5 g, 37 mmol). The mixture was heated at reflux overnight. The reaction mixture was cooled with ice-bath and the precipitate was isolated and washed with n-butanol and ether, dried to give the title compound 110 (7.7 g, 71.3%): LCMS: 373 [M+1]⁺; ¹H NMR (DMSO-d₆): δ 1.53 (d, J=6.9 Hz, 3H), 3.87 (s, 3H), 5.51 (m, 1H), 7.20 (d, J=7.2 Hz, 1H), 7.31 (t, J=7.2 Hz, 3H), 7.42 (d, J=7.2 Hz, 2H), 7.93 (t, J=8.4 Hz, 3H), 8.00 (d, J=8.4 Hz, 2H), 8.09 (s, 1H), 12.20 (s, 1H).

### Step 4g. (4-(4-((R)-1-phenylethylamino)-7H-pyrrolo[2,3-d]pyrimidin-6-yl)phenyl)methanol (Compound 111)

To a suspension of compound 110 (6.15 g, 16.5 mmol) in anhydrous THF (400 mL) was added LiAlH₄ (1.88 g, 0.0495 mol) in portions. The resulting mixture was heated at reflux for 30 minutes. The mixture was cooled to room temperature and H₂O (1.88 mL) was added and followed by addition of 15% aqueous NaOH (1.88 mL) and H₂O (5.64 mL). The precipitate was removed by filtration and the filtrate was concentrated. The residue was suspended in water and the precipitate was collected and dried to give the title compound 111 (4.28 g, 75.3%): LCMS: 345 [M+1]⁺; ¹H NMR (DMSO-d₆): δ 1.54 (d, J=7.2 Hz, 3H), 4.53 (d, J=6.0 Hz, 2H), 5.20 (t, J=6.0 Hz, 1H), 5.50 (m, 1H), 7.08 (s, 1H), 7.20 (t, J=7.5 Hz, 1H), 7.30 (t, J=7.5 Hz, 2H), 7.40 (t, J=8.1 Hz, 4H), 7.76 (t, J=8.4 Hz, 3H), 8.05 (s, 1H), 11.99 (s, 1H).

### Step 4h. 6-(4-(Chloromethyl)phenyl)-N—((R)-1-phenylethyl)-7H-pyrrolo[2,3-d]pyrimidin-4-amine (Compound 112)

To a solution of SOCl₂ (8.85 g, 74.0 mmol) in toluene (50 mL) at −10° C. was added compound 111 in portions. The mixture was warmed to 0° C. and stirred for 2 hours. The reaction mixture was filtered and the solid was washed with toluene and ether to give crude product. The crude product was suspended in water and treated with saturated aqueous NaHCO₃ until pH>7. The solid was isolated and washed with water, dried to give the title compound 112 (1.8 g, 67.0%): LCMS: 363 [M+1]⁺; ¹H NMR (DMSO-d₆): δ 1.54 (d, J=6.9 Hz, 3H), 4.79 (s, 2H), 5.50 (m, 1H), 7.14 (s, 1H), 7.20 (d, J=7.2 Hz, 1H), 7.30 (t, J=7.2 Hz, 2H), 7.42 (d, J=6.9 Hz, 2H), 7.49 (d, J=8.4 Hz, 2H) 7.78 (d, J=7.8 Hz, 2H), 7.82 (d, J=8.4 Hz, 1H) 8.07 (s, 1H), 12.06 (s, 1H).

### Step 4i. (R)-Ethyl 2-(4-(4-(1-phenylethylamino)-7H-pyrrolo[2,3-d]-pyrimidin-6-yl)benzylamino)acetate (Compound 113-1)

To a mixed of DMF (60 mL), MeOH (30 mL) and KOH (448.0 mg, 8.0 mmol) was added ethyl 2-aminoacetate hydrochloride (1.11 g, 8.0 mmol). The resulting mixture was stirred at room temperature for 10 minutes. MeOH was removed at 40° C. under reduced pressure and compound 112 (724.0 mg, 2.0 mmol) was added. The resulting mixture was stirred at room temperature overnight. DMF was removed under reduced pressure and the residue was suspended in water. The resulting solid was collected and dried to give product 113-1 (285 mg, 33%). LCMS: 430 [M+1].

### Step 4j. (R)—N-hydroxy-2-(4-(4-(1-phenylethylamino)-7H-pyrrolo[2,3-d-]pyrimidin-6-yl)benzylamino)acetamide (Compound 1)

A mixture of compound 113-1 (285 mg, 0.66 mmol) and NH₂OH/MeOH (5 mL, 8.85 mmol) was stirred at room temperature for 0.5 h. The reaction mixture was neutralized with AcOH and concentrated. The residue was suspended in water and resulting precipitate was isolated and dried to give crude product. This product was purified by preparative HPLC to give compound 1 as a pale yellow solid (220 mg, 80%). LCMS: 417 [M+1], ¹H NMR (DMSO-d₆): δ 1.52 (d, J=6.3 Hz, 3H), 3.02 (s, 2H), 3.67 (s, 2H), 5.47 (m, 1H), 7.06 (s, 1H), 7.17 (t, J=6.9 Hz, 1H), 7.28 (m, 2H), 7.39 (m, 4H), 7.70 (m, J=7.8 Hz, 2H), 7.78 (d, J=8.1 Hz, 1H) 8.03 (s, 1H), 8.80 (s, 1H), 10.41 (s, 1H), 11.99 (s, 1H).

### Example 5

### Preparation of (R)—N-Hydroxy-3-(4-(4-(1-phenylethylamino)-7H-pyrrolo[2,3-d]pyrimidin-6-yl)benzylamino)-propanamide (Compound 2)

### Step 5a. (R)-Ethyl 3-(4-(4-(1-phenylethylamino)-7H-pyrrolo[2,3-d]-pyrimidin-6-yl)benzylamino)propanoate (Compound 113-2)

The title compound 113-2 was prepared (190 mg, 53%) from compound 112 (290.0 mg, 0.8 mmol) and ethyl 3-amino-propanoate hydrochloride (368 mg, 2.4 mmol) using a procedure similar to that described for compound 113-1 (Example 4): LCMS: 444 [M+1].

### Step 5b. (R)—N-Hydroxy-3-(4-(4-(1-phenylethylamino)-7H-pyrrolo[2,3-d]pyrimidin-6-yl)benzylamino)-propanamide (Compound 2)

The title compound 2 was prepared as a pale yellow solid (45 mg, 24%) from compound 113-2 (190.0 mg, 0.43 mmol) and NH₂OH/MeOH (2 mL, 3.43 mmol) using a procedure similar to that described for compound 1 (Example 4): LCMS: 431 [M+1]⁺, ¹H NMR (DMSO-d₆): δ 1.52 (d, J=6.9 Hz, 3H), 2.14 (t, J=7.2 Hz, 2H), 2.70 (t, J=7.2 Hz, 2H), 3.69 (s, 2H), 5.50 (m, 1H), 7.07 (s, 1H), 7.19 (t, J=6.9 Hz, 1H), 7.30 (t, J=7.2 Hz, 2H), 7.36 (d, J=7.8 Hz, 2H), 7.42 (d, J=7.8 Hz, 2H), 7.74 (m, 3H), 8.05 (s, 1H), 11.97 (s, 1H).

### Example 6

### (R)—N-Hydroxy-2-(4-(4-(4-(1-phenylethylamino)-7H-pyrrolo[2,3-d]pyrimidin-6-yl)benzyl)piperazin-1-yl)acetamide (Compound 11)

### Step 6a. (R)—N-(1-Phenylethyl)-6-(4-(piperazin-1-ylmethyl)phenyl)-7H-pyrrolo[2,3-d]pyrimidin-4-amine (Compound 301)

A mixture of compound 112 (0.1 g, 0.27 mmol) and piperazine (0.21 g, 2.7 mmol) in DMF (20 mL) was stirred at 20° C. for 1.5 hours. The solvent was removed under reduce pressure and the residue was washed with water, dried and purified by HPLC to obtain the title compound 301 as a yellow solid (0.10 g, 87.8%): LCMS: 413 [M+1]⁺.

### Step 6b. (R)-Ethyl 2-(4-(4-(4-(1-phenylethylamino)-7H-pyrrolo[2,3-d]pyrimidin-6-yl)benzyl)piperazin-1-yl)acetate (Compound 302-11)

A mixture of compound 301 (0.25 g, 0.61 mmol), ethyl 2-bromoacetate (0.11 g, 0.66 mmol), triethylamine (0.25 g, 2.44 mmol) in DMF (10 mL) was stirred at 25-30° C. overnight. The solvent was evaporated under reduce pressure to give crude residue 302-11 (0.30 g, LCMS: 499 [M+1]⁺) which was used in the next step directly without further purification.

### Step 6c. (R)—N-Hydroxy-2-(4-(4-(4-(1-phenylethylamino)-7H-pyrrolo[2,3-d]pyrimidin-6-yl)benzyl)piperazin-1-yl)acetamide (Compound 11)

To a solution of hydroxylamine in methanol (4.0 mL, 7.1 mmol) was added compound 302-11 (0.30 g, 0.62 mmol). The reaction mixture was stirred at 25° C. for 20 minutes. The reaction was monitored by TLC. The mixture was neutralized with acetic acid and concentrated under reduce pressure. The residue was purified by preparative HPLC to give the title compound 11 as a white solid (60 mg, 21%): LCMS: 486 [M+1]⁻; ¹H NMR (DMSO-d₆): δ 1.32 (d, J=6.9 Hz, 3H), 2.43 (m, 8H), 2.83 (s, 2H), 3.44 (s, 2H), 5.47 (m, 1H), 7.05 (s, 1H), 7.19 (m, 1H), 7.29 (m, 5H), 7.40 (d, J=7.2 Hz, 3H), 7.71 (d, J=8.1 Hz, 2H), 7.76 (d, J=8.1 Hz, 1H), 8.02 (s, 1H), 11.96 (s, 1H).

### Example 7

### (R)—N-Hydroxy-3-(4-(4-(4-(1-phenylethylamino)-7H-pyrrolo[2,3-d]pyrimidin-6-yl)benzyl)piperazin-1-yl)propanamide (Compound 12)

### Step 7a. (R)-Methyl 3-(4-(4-(4-(1-phenylethylamino)-7H-pyrrolo[2,3-d]pyrimidin-6-yl)benzyl)piperazin-1-yl)propanoate (compound 302-12)

The title compound 302-12 was prepared (0.31 g) from compound 301 (0.44 g, 1.07 mmol), methyl 3-bromopropanoate (0.20 g, 1.17 mmol) and triethylamine (0.43 g, 4.25 mmol) in DMF (9 mL) using a procedure similar to that described for compound 302-11 (Example 6): LCMS: 499 [M+1]⁺.

### Step 7b. (R)—N-Hydroxy-3-(4-(4-(4-(1-phenylethylamino)-7H-pyrrolo[2,3-d]pyrimidin-6-yl)benzyl)piperazin-1-yl)propanamide (Compound 12)

The title compound 12 was prepared as a white solid (80 mg, 26%) from compound 302-12 (0.31 g, 0.62 mmol) using a procedure similar to that described for compound 11 (Example 6): LCMS: 500 [M+1]⁺; ¹H NMR (DMSO-d₆): δ 1.62 (d, J=7.2 Hz, 3H), 2.29 (t, J=7.2 Hz, 2H), 2.54 (m, 8H), 2.67 (t, J=7.2 Hz, 3H), 3.56 (s, 2H), 5.47 (m, 1H), 7.00 (s, 1H), 7.19 (m, 1H), 7.29 (m, 5H), 7.40 (d, J=7.2 Hz, 3H), 7.71 (d, J=8.1 Hz, 2H), 7.76 (d, J=8.1 Hz, 1H), 8.02 (s, 1H), 11.96 (s, 1H).

### Example 8

### (R)—N-Hydroxy-4-(4-(4-(4-(1-phenylethylamino)-7H-pyrrolo[2,3-d]pyrimidin-6-yl)benzyl)piperazin-1-yl)butanamide (Compound 13)

### Step 8a. (R)-Ethyl 4-(4-(4-(4-(1-phenylethylamino)-7H-pyrrolo[2,3-d]pyrimidin-6-yl)benzyl)piperazin-1-yl)butanoate (Compound 302-13)

The title compound 302-13 was prepared (0.39 g) from compound 301 (0.30 g, 0.74 mmol), ethyl 4-bromobutanoate (0.28 g, 0.82 mmol), triethylamine (0.29 g, 2.9 mmol) and DMF (9.5 mL) using a procedure similar to that described for compound 302-11 (Example 6): LCMS: 527 [M+1]⁺.

### Step 8b. (R)—N-Hydroxy-4-(4-(4-(4-(1-phenylethylamino)-7H-pyrrolo[2,3-d]pyrimidin-6-yl)benzyl)piperazin-1-yl)butanamide (Compound 13)

The title compound 13 was prepared as a white solid (20 mg, 5%) from compound 302-13 (0.39 g, 0.74 mmol) using a procedure similar to that described for compound 11 (Example 6): LCMS: 514 [M+1]; ¹H NMR (DMSO-d₆): δ 1.53 (d, J=7.2 Hz, 3H), 1.61 (m, 2H), 1.95 (t, J=7.2 Hz, 2H), 2.37 (m, 8H), 3.46 (s, 2H), 5.48 (m, 1H), 7.08 (s, 1H), 7.17 (m, 1H), 7.29 (m, 5H), 7.43 (d, J=6.9 Hz, 3H), 7.74 (d, J=8.4 Hz, 2H), 7.80 (d, J=8.4 Hz, 1H), 8.05 (s, 1H), 12.00 (s, 1H).

### Example 9

### (R)—N-Hydroxy-5-(4-(4-(4-(1-phenylethylamino)-7H-pyrrolo[2,3-d]pyrimidin-6-yl)benzyl)piperazin-1-yl)pentanamide (Compound 14)

### Step 9a. (R)-Methyl 5-(4-(4-(4-(1-phenylethylamino)-7H-pyrrolo[2,3-d]pyrimidin-6-yl)benzyl)piperazin-1-yl)pentanoate (Compound 302-14)

The title compound 302-14 was prepared (0.40 g) from compound 301 (0.31 g, 0.76 mmol), methyl 5-bromopentanoate (0.178 g, 0.91 mmol), triethylamine (0.31 g, 3.1 mmol) and DMF (10 mL) using a procedure similar to that described for compound 302-11 (Example 6): LCMS: 527 [M+1]⁺.

### Step 9b. (R)—N-Hydroxy-5-(4-(4-(4-(1-phenylethylamino)-7H-pyrrolo[2,3-d]pyrimidin-6-yl)benzyl)piperazin-1-yl)pentanamide (Compound 14)

The title compound 14 was prepared as a white solid (30 mg, 7%) from compound 302-14 (0.40 g, 0.76 mmol) using a procedure similar to that described for compound 11 (Example 6): LCMS: 528 [M+1]⁺; ¹H NMR (DMSO-d₆): δ 1.29 (m, 2H), 1.38 (m, 2H), 1.46 (d, J=7.2 Hz, 3H), 1.86 (t, J=7.2 Hz, 2H), 2.16 (t, J=3.9 Hz, 2H) 2.30 (m, 8H), 3.39 (s, 2H), 5.43 (m, 1H), 7.0 (s, 1H), 7.12 (m, 1H), 7.26 (m, 5H), 7.35 (d, J=7.5 Hz, 3H), 7.76 (d, J=8.4 Hz, 2H), 7.80 (d, J=8.4 Hz, 1H), 7.98 (s, 1H).

### Example 10

### (R)—N-Hydroxy-6-(4-(4-(4-(1-phenylethylamino)-7H-pyrrolo[2,3-d]pyrimidin-6-yl)benzyl)piperazin-1-yl)hexanamide (Compound 15)

### Step 10a. (R)-Ethyl 6-(4-(4-(4-(1-phenylethylamino)-7H-pyrrolo[2,3-d]pyrimidin-6-yl)benzyl)piperazin-1-yl)hexanoate (Compound 302-15)

The title compound 302-15 was prepared (0.41 g) from compound 301 (0.30 g, 0.73 mmol), ethyl 6-bromohexanoate (0.21 g, 0.87 mmol), triethylamine (0.29 g, 2.9 mmol) and DMF (8 mL) using a procedure similar to that described for compound 302-11 (Example 6): LCMS: 555 [M+1]⁺.

### Step 10b. (R)—N-Hydroxy-6-(4-(4-(4-(1-phenylethylamino)-7H-pyrrolo[2,3-d]pyrimidin-6-yl)benzyl)piperazin-1-yl)hexanamide (Compound 15)

The title compound 15 was prepared as a white solid (80 mg, 20%) from compound 302-15 (0.41 g, 0.74 mmol) using a procedure similar to that described for compound 11 (Example 6): LCMS: 542 [M+1]⁺; ¹H NMR (DMSO-d₆): δ 1.15 (m, 2H), 1.34 (m, 2H), 1.41 (m, 2H), 1.51 (d, J=6.9 Hz, 3H), 1.91 (t, J=6.9 Hz, 2H), 2.20 (t, J=6.9 Hz, 2H) 2.35 (m, 8H), 3.34 (s, 2H), 5.48 (m, 1H), 7.6 (s, 1H), 7.18 (m, 1H), 7.29 (m, 4H), 7.41 (d, J=7.2 Hz, 2H), 7.72 (d, J=8.1 Hz, 2H), 7.79 (d, J=8.4 Hz, 1H), 8.03 (s, 1H), 8.65 (s, 1H), 10.30 (s, 1H), 11.98 (s, 1H).

### Example 11

### (R)—N-Hydroxy-7-(4-(4-(4-(1-phenylethylamino)-7H-pyrrolo[2,3-d]pyrimidin-6-yl)benzyl)piperazin-1-yl)heptanamide (Compound 16)

### Step 11a. (R)-Ethyl 7-(4-(4-(4-(1-phenylethylamino)-7H-pyrrolo[2,3-d]pyrimidin-6-yl)benzyl)piperazin-1-yl)heptanoate (Compound 302-16)

The title compound 302-16 was prepared (0.13 g, 23%) from compound 301 (0.41 g, 1.0 mmol), ethyl 7-bromoheptanoate (0.237 g, 1 mmol), triethylamine (0.40 g, 0.40 mmol) and DMF (6 mL) using a procedure similar to that described for compound 302-11 (Example 6): LCMS: 569 [M+1]⁺.

### Step 11b. (R)—N-Hydroxy-7-(4-(4-(4-(1-phenylethylamino)-7H-pyrrolo[2,3-d]pyrimidin-6-yl)benzyl)piperazin-1-yl)heptanamide (Compound 16)

The title compound 16 was prepared as a brown solid (84 mg, 66%) from compound 302-16 (0.13 g, 0.23 mmol) using a procedure similar to that described for compound 11 (Example 6): LCMS: 556 [M+1]⁺; ¹H NMR (DMSO-d₆): δ 1.23 (m, 4H), 1.46 (m, 4H), 1.51 (d, J=7.2 Hz, 3H), 1.92 (t, J=7.8 Hz, 2H), 2.50-2.80 (m, 8H), 3.56 (s, 2H), 5.48 (m, 1H), 7.09 (s, 1H), 7.18 (m, 1H), 7.26 (m, 2H), 7.40 (m, 5H), 7.74 (d, J=7.8 Hz, 2H), 7.81 (d, J=8.1 Hz, 1H), 8.66 (s, 1H), 10.34 (s, 1H), 12.00 (s, 1H).

### Example 12

### (R)—N-Hydroxy-4-(4-(4-(1-phenylethylamino)-7H-pyrrolo[2,3-d]pyrimidin-6-yl)phenoxy)propanamide (Compound 19)

### Step 12a. (R)-Methyl-4-(4-(4-(1-phenylethylamino)-7H-pyrrolo[2,3-d]pyrimidin-6-yl)phenoxy)butanoate (Compound 407-19)

To a mixture of compound 406 (250 mg, 0.75 mmol) and K₂CO₃ (160 mg, 1.2 mmol) in N,N-dimethylformamide (1.5 mL) was added methyl 4-bromobutyrate (130 mg, 0.75 mmol) and the resulting mixture was stirred at 40° C. for 20 h. Water (5 ml) was added and the mixture was extracted with ethyl acetate (25 mL×4), dried and concentrated. The residue was purified by column chromatography to afford the product 407-19 as a white solid (202 mg, 63% yield): LC-MS: 431 (M+1); ¹H NMR (DMSO-d₆): δ 1.49 (d, J=6.6 Hz, 3H), 1.90-1.93 (m, 2H), 2.11 (t, J=7.2 Hz, 2H), 3.60 (s, 3H), 4.02 (t, J=6.0 Hz, 2H), 5.43-5.48 (m, 1H), 6.92-6.98 (m, 2H), 7.16-7.18 (m, 1H), 7.24-7.29 (m, 2H), 7.39 (d, J=8.4 Hz, 2H), 7.65-7.71 (m, 3H), 8.00 (s, 1H), 11.87 (s, 1H).

### Step 12b. (R)—N-Hydroxy-4-(4-(4-(1-phenylethylamino)-7H-pyrrolo[2,3-d]pyrimidin-6-yl)phenoxy)propanamide (Compound 19)

To a flask containing compound 407-19 (180 mg, 0.45 mmol) was added the solution of hydroxylamine in methanol (2.0 mL). The mixture was stirred at room temperature for 1 hour. The reaction mixture was neutralized with conc. HCl and concentrated. The residue was purified by column chromatography to afford the product 19 as a white solid (60 mg, 34% yield). LC-MS: 432 (M+1); ¹H NMR (DMSO-d₆): δ 1.49 (d, J=6.6 Hz, 3H), 1.89-1.93 (m, 2H), 2.10 (t, J=7.2 Hz, 2H), 3.97 (t, J=6.0 Hz, 2H), 5.43-5.48 (m, 1H), 6.92-6.98 (m, 2H), 7.16-7.18 (m, 1H), 7.24-7.29 (m, 2H), 7.38-7.41 (d, J=8.4 Hz, 2H), 7.65-7.71 (m, 3H), 7.99 (s, 1H), 8.70 (s, 1H), 10.41 (s, 1H), 11.88 (s, 1H).

### Example 13

### (R)—N-Hydroxy-5-(4-(4-(1-phenylethylamino)-7H-pyrrolo[2,3-d]pyrimidin-6-yl)phenoxy)pentanamide (Compound 20)

### Step 13a. (R)-Methyl-5-(4-(4-(1-phenylethylamino)-7H-pyrrolo[2,3-d]pyrimidin-6-yl)phenoxy)pentanoate (Compound 407-20)

The title compound 407-20 was prepared as a white solid (150 mg, 87%) from compound 406 (130 mg, 0.39 mmol), K₂CO₃ (110 mg, 0.8 mmol), methyl 5-bromovalerate (76 mg, 0.39 mmol) using a procedure similar to that described for compound 407-19 (Example 12): LC-MS: 445 (M+1); ¹H NMR (DMSO-d₆): δ 1.47-1.54 (m, 5H), 1.88-1.94 (m, 2H), 2.36 (t, J=7.5 Hz, 2H), 3.58 (s, 3H), 4.30-4.33 (m, 2H), 5.46-5.50 (m, 1H), 6.91-6.98 (m, 2H), 7.16-7.18 (m, 1H), 7.24-7.30 (m, 2H), 7.40 (d, J=7.5 Hz, 2H), 7.65-7.68 (m, 3H), 8.00 (s, 1H), 11.87 (s, 1H).

### Step 13b. (R)—N-Hydroxy-5-(4-(4-(1-phenylethylamino)-7H-pyrrolo[2,3-d]pyrimidin-6-yl)phenoxy)pentanamide (Compound 20)

The title compound 20 was prepared as a white solid (110 mg, 73%) from compound 407-20 (150 mg, 0.35 mmol) using a procedure similar to that described for compound 19 (Example 12): LC-MS: 446 (M+1); ¹H NMR (DMSO-d₆): δ 1.50 (d, J=7.2 Hz, 3H), 1.65-1.66 (m, 4H), 1.98-2.02 (m, 2H), 3.97 (m, 2H), 5.44-5.49 (m, 1H), 6.93-6.99 (m, 2H), 7.16-7.18 (m, 1H), 7.25-7.30 (m, 2H), 7.39-7.41 (d, J=8.4 Hz, 2H), 7.66-7.71 (m, 3H), 8.00 (s, 1H), 8.70 (s, 1H), 10.42 (s, 1H), 11.87 (s, 1H).

### Example 14

### (R)—N¹-Hydroxy-N¹-(4-(4-(1-phenylethylamino)-7H-pyrrolo[2,3-d]pyrimidin-6-yl)phenyl)succinamide (Compound 24)

### Step 14a. Ethyl 2-amino-5-(4-nitrophenyl)-1H-pyrrole-3-carboxylate (Compound 502)

Under a nitrogen atmosphere, compound 104 (16.7 g, 100 mmol) was introduced into 25 mL of ethanol at 0˜5° C. followed by sodium ethanolate (6.8 g, 100 mmol). The yellow suspension was stirred for 20 minutes and compound 501 (12.2 g, 50 mmol) was added. The resulting mixture was stirred for 24 hours at room temperature and concentrated under reduced pressure. The residue was dissolved in ethyl acetate and washed with water and brine. The aqueous phase was extracted three times with ethyl acetate. The combined organic layers were dried over MgSO₄ and evaporated to afford crude product 502 (12.1 g, 79.5%). LC-MS: 276 (M+1), ¹H NMR (DMSO-d₆) δ1.26 (t, J=7.2 Hz, 3H), 4.17 (q, J₁=7.2 Hz, J2=7.2 Hz. 2H), 5.98 (s, 1H), 6.91 (s, 1H), 7.68 (d, J=9.0 Hz, 2H), 8.13 (d, J=9.0 Hz, 2H), 110.1 (s, 1H).

### Step 14b. 6-(4-Nitrophenyl)-7H-pyrrolo[2,3-d]pyrimidin-4-ol (Compound 503)

A mixture of 502 (5.0 g, 18.2 mmol), formamide (36 mL) and formic acid (6 mL) in DMF (10 mL) were stirred at 150° C. for 22 hours. The mixture was cooled to room temperature and diluted with water. The resulting precipitate was filtered and washed with water, isopropanol, ether and dried to obtain a gray solid 503 (3.24 g, 69.4%). LC-MS: 257 (M+1), ¹H NMR (DMSO-d₆) δ7.28 (s, 1H), 7.95 (s, 1H), 8.11 (d, J=9.0 Hz, 2H), 8.26 (d, J=9.0 Hz, 2H), 11.98 (s, 1H), 12.67 (s, 1H).

### Step 14c. 4-Chloro-6-(4-nitrophenyl)-7H-pyrrolo[2,3-d]pyrimidine (Compound 504)

A mixture of 503 (0.52 g, 2.03 mmol) and phosphorus oxychloride (10 mL) were refluxed for 3 hours. The dark-brown suspension was concentrated to remove the phosphorus oxychloride. The residue was diluted with ethyl acetate and the organic layer was washed with saturated aqueous NaHCO₃, dried over MgSO₄ and evaporated to give the product 504 as a yellow solid (0.13 g, 22.2%). LC-MS: 275 (M+1), ¹H NMR (DMSO-d₆) δ7.42 (s, 1H), 8.28˜8.37 (m, 4H), 8.67 (s, 1H), 13.31 (s, 1H).

### Step 14d. (R)-6-(4-Nitrophenyl)-N-(1-phenylethyl)-7H-pyrrolo[2,3-d]pyrimidin-4-amine (Compound 505)

Compound 504 (5.53 g, 20.1 mmol) was suspended in n-butanol (110 mL) and treated with (R)-phenethylamine (4.9 g, 40.3 mmol). The mixture was heated at 145° C. for 24 h. The reaction mixture was cooled in an ice bath and the solid was filtered and washed with cold n-butanol and ether to obtain a black product 505 (4.2 g, 58.2%). LC-MS: 360 (M+1), ¹H NMR (DMSO-d₆) δ1.52 (d, J=6.6 Hz, 3H), 5.52 (m, 1H), 7.21˜7.49 (m, 6H), 8.00˜8.32 (m, 6H), 13.36 (s, 1H).

### Step 14e. (R)-6-(4-Aminophenyl)-N-(1-phenylethyl)-7H-pyrrolo[2,3-d]pyrimidin-4-amine (Compound 506)

A mixture of compound 505 (5.44 g, 15.14 mmol), iron dust (8.48 g, 0.15 mol) and concentrated HCl (1 mL) in ethanol (120 mL) and water (12 mL) was refluxed for 2 hours. The mixture was adjusted to pH=12 with aqueous NaOH and iron dust was removed by filtration. The filtrate was concentrated to yield a residue which was purified by column chromatography to give product 506 as a yellow solid (1.48 g, 29.7%). LC-MS: 330 (M+1), ¹H NMR (DMSO-d₆) δ1.52 (d, J=6.6 Hz, 3H), 5.29 (s, 2H), 5.48 (m, 1H), 6.60˜6.63 (m, 2H), 6.81 (s, 1H), 7.18˜7.63 (m, 9H), 7.99 (s, 1H), 11.68 (s, 1H).

### Step 14f. (R)-Methyl 4-oxo-4-(4-(4-(1-phenylethylamino)-7H-pyrrolo[2,3-d]pyrimidin-6-yl)phenyl-amino)butanoate (Compound 507-24)

A solution of succinic acid monomethyl ester (401.6 mg, 3.04 mmol) in SOCl₂ (20 mL) was heated at 80° C. for 4 h. The mixture was allowed to cool and the solvent was removed by evaporation. This mixture was then added dropwise to a suspension of compound 506 (0.5 g, 1.52 mmol) in CH₂Cl₂ (30 mL) and triethylamine (0.86 mL, 6.08 mmol) at 0° C. The mixture was stirred for 2 hours at 0° C. and was diluted with CH₂Cl₂ (150 mL) and washed with water (100 mL×3), dried over MgSO₄. The organic solvent was removed to give crude product 507-24 as a yellow solid (0.7 g) that was used in the next step directly without further purification. LC-MS: 444 (M+1).

### Step 14g. (R)—N¹-Hydroxy-N⁴-(4-(4-(1-phenylethylamino)-7H-pyrrolo[2,3-d]pyrimidin-6-yl)phenyl)succinamide (Compound 24)

A mixture of 507-24 and saturated solution of hydroxylamine in methanol (1.77 mol/L, 5.15 mL) was stirred for 2.5 hours at room temperature. The mixture was adjusted to pH=7˜8 with acetic acid and solvent was removed by evaporation. Water was added to the mixture and the precipitate was filtered and purified to give product 24 as a yellow solid (0.12 g, 17.8% in two steps). LC-MS: 445 (M+1), ¹H NMR (DMSO-d₆) δ1.50 (d, J=6.6 Hz, 3H), 2.29 (t, J=7.5 Hz, 2H), 2.57 (t, J=7.2 Hz, 2H), 5.47 (m, 1H), 6.99 (s, 1H), 7.17˜7.42 (m, 5H), 7.65˜7.76 (m, 5H), 8.02 (s, 1H), 8.72 (s, 1H), 10.06 (s, 1H), 10.43 (s, 1H), 11.91 (s, 1H).

### Example 15

### (R)—N¹-Hydroxy-N⁵-(4-(4-(1-phenylethylamino)-7H-pyrrolo[2,3-d]pyrimidin-6-yl)phenyl)glutaramide (Compound 25)

### Step 15a. (R)-Methyl 5-oxo-5-(4-(4-(1-phenylethylamino)-7H-pyrrolo[2,3-d]pyrimidin-6-yl)phenylamino)pentanoate (Compound 507-25)

The title compound 507-25 was prepared as a red viscous liquid (0.8 g) from compound 506 (0.5 g, 1.52 mmol) and glutaric acid monomethyl ester (222.1 mg, 3.04 mmol) using a procedure similar to that described for compound 507-24 (Example 14): LC-MS: 458 (M+1).

### Step 15b. (R)—N¹-Hydroxy-N⁵-(4-(4-(1-phenylethylamino)-7H-pyrrolo[2,3-d]pyrimidin-6-yl)phenyl)glutaramide (Compound 25)

The title compound 25 was prepared as a yellow solid (0.22 g, 31.6% yield in two steps) from of hydroxylamine in methanol (1.77 mol/L, 3.44 mL) using a procedure similar to that described for compound 24 (Example 14): LC-MS: 459 (M+1), ¹H NMR (DMSO-d₆) δ1.49 (d, J=6.9 Hz, 3H), 1.79 (t, J=7.5 Hz, 2H), 2.00 (t, J=7.2 Hz, 2H), 2.31 (t, J=7.2 Hz, 2H), 5.46 (m, 1H), 6.98 (s, 1H), 7.14˜7.41 (m, 5H), 7.61˜7.75 (m, 5H), 8.01 (s, 1H), 8.68 (s, 1H), 9.87 (s, 1H), 10.37 (s, 1H), 11.90 (s, 1H).

### Example 16

### (R)—N¹-Hydroxy-N⁶-(4-(4-(1-phenylethylamino)-7H-pyrrolo[2,3-d]pyrimidin-6-yl)phenyl)adipamide (Compound 26)

### Step 16a. (R)-Methyl 6-oxo-6-(4-(4-(1-phenylethylamino)-7H-pyrrolo[2,3-d]pyrimidin-6-yl)phenyl-amino)hexanoate (Compound 507-26)

The title compound 507-26 was prepared as a yellow solid (0.44 g) from compound 506 (0.25 g, 0.76 mmol) and adipic acid monomethyl ester (243.5 mg, 1.52 mmol) using a procedure similar to that described for compound 507-24 (Example 14): LC-MS: 472 (M+1).

### Step 16b. (R)—N¹-hydroxy-N⁶-(4-(4-(1-phenylethylamino)-7H-pyrrolo[2,3-d]pyrimidin-6-yl)phenyl)adipamide (Compound 26)

The title compound 26 was prepared as a white solid (0.15 g, 41.8% yield in two steps) from 507-26 (0.31 g, 0.62 mmol) using a procedure similar to that described for compound 24 (Example 14): LC-MS: 473 (M+1), ¹H NMR (DMSO-d₆) δ 1.51 (m, 7H), 1.95 (t, J=6.9 Hz, 2H), 2.30 (t, J=6.6 Hz, 2H), 5.46 (m, 1H), 6.97 (s, 1H), 7.14˜7.41 (m, 5H), 7.61˜7.75 (m, 5H), 8.01 (s, 1H), 8.66 (s, 1H), 9.95 (s, 1H), 10.34 (s, 1H), 11.90 (s, 1H).

### Example 17

### (R)—N¹-Hydroxy-N⁸-(4-(4-(1-phenylethylamino)-7H-pyrrolo[2,3-d]pyrimidin-6-yl)phenyl)octanediamide (Compound 27)

### Step 17a. (R)-Methyl 8-oxo-8-(4-(4-(1-phenylethylamino)-7H-pyrrolo[2,3-d]pyrimidin-6-yl)phenylamino)octanoate (Compound 507-27)

The title compound 507-27 was prepared as a yellow solid (1.12 g) from compound 506 (0.5 g, 1.52 mmol) and suberic acid monomethyl ester (571.9 mg, 3.04 mmol) using a procedure similar to that described for compound 507-24 (Example 14): LC-MS: 500 (M+1).

### Step 17b. (R)—N¹-Hydroxy-N⁸-(4-(4-(1-phenylethylamino)-7H-pyrrolo[2,3-d]pyrimidin-6-yl)phenyl)octanediamide (Compound 27)

The title compound 27 was prepared as a white solid (0.2 g, 26.3% yield in two steps) from 507-27 using a procedure similar to that described for compound 24 (Example 14). LC-MS: 501 (M+1), ¹H NMR (DMSO-d₆) δ1.26˜1.58 (m, 11H), 1.89 (t, J=7.2 Hz, 2H), 2.28 (t, J=7.2 Hz, 2H), 5.46 (m, 1H), 6.98 (s, 1H), 7.13˜7.41 (m, 5H), 7.61˜7.75 (m, 5H), 8.01 (s, 1H), 8.63 (s, 1H), 9.94 (s, 1H), 10.30 (s, 1H), 11.90 (s, 1H).

### Example 18

### (R)—N-(2-(2-(Hydroxyamino)-2-oxoethylamino)ethyl)-4-(4-(1-phenylethylamino)-7H-pyrrolo[2,3-d]pyrimidin-6-yl)benzamide (Compound 28)

### Step 18a. (R)—N-(2-Aminoethyl)-4-(4-(1-phenylethylamino)-7H-pyrrolo[2,3-d]pyrimidin-6-yl)benzamide (Compound 601)

Compound 110 (2.0 g, 5.37 mmol) in ethane-1,2-diamine (120 mL) was stirred at 70° C. for 22 hours. The mixture was concentrated under reducing pressure. The residue was dissolved in 3 mL ethanol and diluted with ether. The resulting precipitate was filtered, dried to obtain a yellow solid, 601 (2.0 g, 93.0%): LC-MS: 401 [M+1]⁺, ¹H NMR (DMSO-d₆): δ 1.54 (d, 3H), 2.53 (t, J=1.8 Hz, 1H), 2.72 (t, J=6.0 Hz, 2H), 3.30 (m, J=6.0 Hz, 2H), 5.51 (m, J=6.6 Hz, J=7.8 Hz, 2H), 7.22 (s, 1H), 7.24 (d, J=4.2 Hz, 1H), 7.31 (t, J=7.2 Hz, 2H), 7.44 (d, J=7.5 Hz, 2H), 7.8 (s, 1H), 7.89 (d, J=7.2 Hz, 2H), 7.93 (s, 2H), 7.96 (s, 1H), 8.09 (s, 1H), 8.49 (t, J=5.7 Hz, 1H).

### Step 18b. (R)-ethyl 2-(2-(4-(4-(1-phenylethylamino)-7H-pyrrolo[2,3-d]pyrimidin-6-yl)benzamido)ethylamino)acetate (Compound 602-28)

A solution of 601 (1.0 g, 2.5 mmol) and ethyl 2-bromoacetate (0.42 g, 2.5 mmol) in N,N-dimethylformamide (25 mL) was stirred at room temperature for 4 hours. The solvent was removed and the residue was purified by silica gel column chromatography to obtained 602-28 (0.79 g, 43.2%). LC-MS: 487 [M+1]⁺.

### Step 18c. (R)—N-(2-(2-(Hydroxyamino)-2-oxoethylamino)ethyl)-4-(4-(1-phenylethylamino)-7H-pyrrolo[2,3-d]pyrimidin-6-yl)benzamide (Compound 28)

The mixture of 602-28 (0.423 g, 0.87 mmol) and hydroxylamine in methanol (1.77 mol/L, 4.91 mL) were stirred for 2.5 hours at room temperature. The mixture was adjusted to pH=7˜8 with acetic acid and solvent was removed. The resulting mixture was diluted with water, filtered and the solid was purified to give compound 28 as a yellow solid (0.09 g, 21.8%): LC-MS: 474 [M+1]⁺, ¹H NMR (DMSO-d₆+D₂O): δ 1.48 (d, J=6.9 Hz, 3H), 2.60 (t, J=6.0 Hz, 2H), 3.04 (s, 2H), 3.31 (t, 2H), 5.37 (m, 1H), 7.14˜7.38 (m, 6H), 7.84 (s, 4H), 7.98 (s, 1H).

### Example 19

### (R)—N-(2-(3-(Hydroxyamino)-3-oxopropylamino)ethyl)-4-(4-(1-phenylethyl-amino)-7H-pyrrolo[2,3-d]pyrimidin-6-yl)benzamide (Compound 29)

### Step 19a. (R)-Methyl 3-(2-(4-(4-(1-phenylethylamino)-7H-pyrrolo[2,3-d]pyrimidin-6-yl)benzamido)ethylamino)propanoate (Compound 602-29)

The title compound 602-29 was prepared as a solid (0.29 g, 23.4%) from compound 601 (1.0 g, 2.5 mmol) and methyl 3-bromopropanoate (0.42 g, 2.5 mmol) in N,N-dimethylformamide (25 mL) using a procedure similar to that described for compound 602-28 (Example 18): LCMS: 487 [M+1]⁺.

### Step 19b. (R)—N-(2-(3-(Hydroxyamino)-3-oxopropylamino)ethyl)-4-(4-(1-phenylethylamino)-7H-pyrrolo[2,3-d]pyrimidin-6-yl)benzamide (Compound 29)

The title compound 29 was prepared as a yellow solid (0.04 g, yield 13.9%) from compound 602-29 (0.29 g, 0.59 mmol) and hydroxylamine in methanol (1.77 mol/L, 6 mL) using a procedure similar to that described for compound 28 (Example 18): LC-MS: 488 [M+1], ¹H NMR (DMSO-d₆+D₂O): δ 1.50 (d, J=6.9 Hz, 3H), 2.15 (t, J₁=6.3 Hz, J₂=7.2 Hz, 2H), 2.76 (m, 4H), 3.35 (m, 2H), 5.44 (m, 1H), 7.16 (d, J=6.9 Hz, 2H), 7.27 (t, J=7.5 Hz, 2H), 7.39 (d, J=7.2 Hz, 2H), 7.86 (m, 4H), 8.03 (s, 1H).

### Example 20

### (R)—N-(2-(6-(Hydroxyamino)-6-oxohexylamino)ethyl)-4-(4-(1-phenylethylamino)-7H-pyrrolo[2,3-d]pyrimidin-6-yl)benzamide (Compound 30)

### Step 20a. (R)-Ethyl 6-(2-(4-(4-(1-phenylethylamino)-7H-pyrrolo[2,3-d]pyrimidin-6-yl)benzamido)ethylamino)hexanoate (Compound 602-30)

The title compound 602-30 was prepared (0.26 g, 24.0%) from compound 601 (0.8 g, 2.0 mmol) and ethyl 6-bromohexanoate (0.446 g, 2.0 mmol) in N,N-dimethylformamide (20 mL) using a procedure similar to that described for compound 602-28 (Example 18): LC-MS: 543 [M+1]⁺.

### Step 20b. (R)—N-(2-(6-(hydroxyamino)-6-oxohexylamino)ethyl)-4-(4-(1-phenylethylamino)-7H-pyrrolo[2,3-d]pyrimidin-6-yl)benzamide (compound 30)

The title compound 30 was prepared as a yellow solid (0.07 g, 27.6%) from compound 602-30 (0.260 g, 0.48 mmol) and the solution of hydroxylamine in methanol (1.77 mol/L, 6 mL) using a procedure similar to that described for compound 28 (Example 18): LC-MS: 530 [M+1]⁺, ¹H NMR (DMSO-d₆+D₂O): δ 1.23 (m, 2H), 1.48 (s, 7H), 1.94 (s, 2H), 2.83 (s, 2H), 3.03 (s, 2H), 3.52 (s, 2H), 5.38 (s, 1H), 7.00˜7.40 (m, 6H), 7.70˜8.10 (m, 5H).

### Example 21

### (R)—N-(2-(7-(Hydroxyamino)-7-oxoheptylamino)ethyl)-4-(4-(1-phenyl ethyl-amino)-7H-pyrrolo[2,3-d]pyrimidin-6-yl)benzamide (Compound 31)

### Step 21a. (R)-Ethyl 7-(2-(4-(4-(1-phenylethylamino)-7H-pyrrolo[2,3-d]pyrimidin-6-yl)benzamido)ethylamino)heptanoate (Compound 602-31)

The title compound 602-31 was prepared (0.40 g, 19.0%) from compound 601 (1.5 g, 3.75 mmol) and ethyl 7-bromoheptanoate (0.888 g, 3.75 mmol) in N,N-dimethylformamide (50 mL) using a procedure similar to that described for compound 602-28 (Example 18): LC-MS: 557 [M+1].

### Step 21b. (R)—N-(2-(7-(Hydroxyamino)-7-oxoheptylamino)ethyl)-4-(4-(1-phenylethylamino)-7H-pyrrolo[2,3-d]pyrimidin-6-yl)benzamide (Compound 31)

The title compound 31 was prepared as a yellow solid (0.072 g, 18.7%) from compound 602-31 (0.396 g, 0.71 mmol) and hydroxylamine in methanol (1.77 mol/L, 8 mL) using a procedure similar to that described for compound 28 (Example 18): LC-MS: 544 [M+1]⁺, ¹H NMR (DMSO-d₆+D₂O): δ 1.20 (s, 4H), 1.48 (s, 7H), 1.93 (s, 2H), 2.69 (s, 2H), 2.89 (s, 2H), 3.46 (s, 2H), 5.37 (s, 1H), 7.10˜7.50 (m, 6H), 7.85 (s, 4H), 7.99 (s, 1H).

### Example 22

### Preparation of (R)—N-hydroxy-6-(4-(4-(1-phenylethylamino)-7H-pyrrolo[2,3-d]pyrimidin-6-yl)benzylamino)hexanamide (Compound 32)

### Step 22a. (R)-Methyl 6-(4-(4-(1-phenylethylamino)-7H-pyrrolo[2,3-d]pyrimidin-6-yl)benzylamino)hexanoate (Compound 113-32)

To a mixed solution of DMF (10 mL) and MeOH (5 mL) was added KOH (168.0 mg, 3.0 mmol) and methyl 6-aminohexanoate hydrochloride (545.0 mg, 3.0 mmol). The mixture was stirred at room temperature for 10 minutes and MeOH was removed at 40° C. under reduced pressure. Compound 112 (363 mg, 1 mmol) was added the above mixture and was stirred at room temperature overnight. DMF was removed under reduced pressure and the residue was suspended in water. The resulting solid was collected and dried to give product 113-32 (280 mg, 59%). LCMS: 472 [M+1]⁺.

### Step 22b. (R)—N-Hydroxy-6-(4-(4-(1-phenylethylamino)-7H-pyrrolo[2,3-d]pyrimidin-6-yl)benzyl-amino)hexanamide (Compound 32)

A mixture of compound 113-32 (280.0 mg, 0.59 mmol) and NH₂OH/MeOH (2.7 mL, 4.75 mmol) was stirred at room temperature for 0.5 hours. The reaction mixture was neutralized with acetic acid and concentrated. The residue was suspended in water and the resulting precipitate was isolated and dried to give crude product that was purified by preparative HPLC to give product 32 as a pale yellow solid (48 mg, 17% yield in two steps). LCMS: 473 [M+1]; ¹H NMR (DMSO-d₆): δ1.27 (m, 2H), 1.46 (m, 4H), 1.52 (d, J=7.2 Hz, 3H), 1.94 (t, J=7.2 Hz, 2H), 2.59 (t, J=7.2 Hz, 2H), 3.81 (s, 2H), 5.47 (m, 1H), 7.09 (s, 1H), 7.19 (t, J=7.5 Hz, 1H), 7.30 (t, J=7.5 Hz, 2H), 7.41 (d, J=7.5 Hz, 4H), 7.76 (m, 3H), 8.05 (s, 1H), 10.32 (s, 1H), 12.00 (s, 1H).

### Example 23

### Preparation of (R)—N-hydroxy-7-(4-(4-(1-phenylethylamino)-7H-pyrrolo[2,3-d]pyrimidin-6-yl)benzylamino)heptanamide (Compound 33)

### Step 23a. (R)-Methyl 7-(4-(4-(1-phenylethylamino)-7H-pyrrolo[2,3-d]pyrimidin-6-yl)benzylamino)heptanoate (Compound 113-33)

The title compound 113-33 was prepared (102 mg, 25%) from compound 112 (300 mg, 0.83 mmol) and 7-amino-heptanoate hydrochloride (487 mg, 2.49 mmol) using a procedure similar to that described for compound 113-32 (Example 22): LCMS: 486 [M+1]⁻.

### Step 23b. (R)—N-hydroxy-7-(4-(4-(1-phenylethylamino)-7H-pyrrolo[2,3-d]pyrimidin-6-yl)benzyl-amino)-heptanamide (Compound 33)

The title compound 33 was prepared as a pale yellow solid (28 mg, 29%) from compound 113-33 (97 mg, 0.2 mmol) and NH₂OH/MeOH (3 mL, 5.31 mmol) using a procedure similar to that described for compound 32 (Example 22): LCMS: 487 [M+1]⁻; ¹H NMR: (DMSO-d₆): δ 1.24 (m, 2H), 1.43 (m, 6H), 1.52 (d, J=7.2 Hz, 3H), 1.93 (t, J=7.5 Hz, 2H), 1.95 (m, 2H), 3.71 (s, 2H), 5.50 (m, 1H), 7.06 (s, 1H), 7.19 (t, J=7.2 Hz, 1H), 7.30 (t, J=7.2 Hz, 2H), 7.40 (d, J=8.1 Hz, 2H), 7.42 (d, J=7.5 Hz, 2H), 7.71 (t, J=8.1 Hz, 3H), 8.05 (s, 1H), 8.62 (s, 1H), 10.29 (s, 1H), 11.95 (s, 1H).

### Example 24

### Preparation of (R)—N-hydroxy-8-(4-(4-(1-phenylethylamino)-7H-pyrrolo[2,3-d]pyrimidin-6-yl)benzylamino)-octanamide (Compound 34)

### Step 24a. (R)-Methyl 8-(4-(4-(1-phenylethylamino)-7H-pyrrolo[2,3-d]pyrimidin-6-yl)benzylamino)octanoate (Compound 113-34)

The title compound 113-34 was prepared as a solid (110 mg, 55%) from compound 112 (145 mg, 0.4 mmol) and 8-aminooctanoate hydrochloride (250 mg, 1.2 mmol) using a procedure similar to that described for compound 113-32 (Example 22): LCMS: 500 [M+1]⁺.

### Step 24b. (R)—N-Hydroxy-8-(4-(4-(1-phenylethylamino)-7H-pyrrolo[2,3-d]pyrimidin-6-yl)benzyl-amino)-octanamide (Compound 34)

The title compound 34 was prepared as a pale yellow solid (41 mg, 37%) from compound 113-34 (110 mg, 0.22 mmol) and NH₂OH/MeOH (5 mL, 8.85 mmol) using a procedure similar to that described for compound 32 (Example 22): LCMS: 501 [M+1]⁻; ¹H NMR: (DMSO-d₆): δ 1.24 (s, 8H), 1.46 (m, 4H), 1.53 (d, J=6.9 Hz, 3H), 1.94 (t, J=6.9 Hz, 2H), 3.70 (s, 2H), 5.50 (m, 1H), 7.07 (s, 1H), 7.20 (t, J=7.2 Hz, 1H), 7.30 (t, J=7.2 Hz, 2H), 7.40 (d, J=8.4 Hz, 2H), 7.43 (d, J=7.2 Hz, 2H), 7.71 (d, J=8.4 Hz, 2H), 7.77 (d, J=8.1 Hz, 1H), 8.06 (s, 1H), 8.67 (s, 1H), 10.33 (s, 1H), 11.98 (s, 1H).

### Example 25

### Preparation of (R)-2-(4-(4-(1-(4-fluorophenyl)ethylamino)-7H-pyrrolo[2,3-d]pyrimidin-6-yl)phenoxy)-N-hydroxyacetamide (Compound 37)

### Step 25a. (R)—N-(1-(4-Fluorophenyl)ethyl)-6-(4-methoxyphenyl)-7H-pyrrolo[2,3-d]pyrimidin-4-amine (Compound 408)

A mixture of compound 404 (2.59 g, 10.0 mmol) and (R)-1-(4-fluorophenyl)ethanamine (2.75 g, 20.0 mmol) in n-BuOH (30 mL) was stirred at 140° C. overnight. The mixture was cooled, filtered, washed with Et₂O to afford the product 408 as a yellow solid (2.3 g, 63%). LCMS: 363 [M+1]⁺.

### Step 25b. (R)-4-(4-(1-(4-Fluorophenyl)ethylamino)-7H-pyrrolo[2,3-d]pyrimidin-6-yl)phenol (Compound 409)

To a solution of compound 408 (2.1 g, 5.6 mmol) in dichloromethane (150 mL) was added dropwise a solution of BBr₃ (5.7 mL, 15.5 mmol) in dichloromethane (190 mL) at 0° C. under nitrogen over 1 hour. After the addition was completed, the mixture was allowed to warm to room temperature and stirred overnight. Then 20 mL of water was added at −20° C. The mixture was warmed to room temperature, extracted with ethyl acetate (150 mL×3), washed with brine, filtered and concentrated to give the product 409 as a yellow solid (1.6 g, 81%). LCMS: 349 [M+1]⁺.

### Step 25f. (R)-Ethyl 2-(4-(4-(1-(4-fluorophenyl)ethylamino)-7H-pyrrolo[2,3-d]pyrimidin-6-yl)phenoxy)acetate (Compound 410-37)

To a mixture of compound 409 (522 mg, 1.5 mmol) and K₂CO₃ (345 mg, 2.5 mmol) in N,N-dimethylformamide (5.0 mL) was added Ethyl 7-bromoheptanoate (356 mg, 1.5 mmol) and the mixture was stirred at 70° C. for 20 hours. DMF was removed under reduced pressure at 50° C. and then 30 mL of ethyl acetate was added. The organic layer was washed with water, dried over anhydrous Na₂SO₄, filtered, concentrated to give compound 410-37 (385 mg, 51%). LCMS: 505 [M+1]⁺.

### Step 25g. (R)-2-(4-(4-(1-(4-Fluorophenyl)ethylamino)-7H-pyrrolo[2,3-d]pyrimidin-6-yl)phenoxy)-N-hydroxyacetamide (Compound 37)

To a flask containing compound 410-37 (170 mg, 0.33 mmol) was added the saturated solution of hydroxylamine in methanol (5.0 mL). The mixture was stirred at room temperature for 30 min. Then it was neutralized to pH 7 using acetic acid and concentrated. The residue was washed with water, evaporated to afford crude product that was purified by column chromatography. The product 37 was obtained as a white solid (40 mg, 25%): LCMS: 492 [M+1]⁺; ¹H NMR (DMSO-d₆): δ 11.29˜1.54 (m, 9H), 1.65˜1.74 (m, 2H), 1.94 (t, J=7.5 Hz, 2H), 3.98 (t, J=6.3 Hz, 2H), 5.47 (t, J=8.1 Hz, 1H), 6.91 (s, 1H), 6.98 (d, J=9.3 Hz, 2H), 7.42˜7.46 (m, 3H), 7.68 (d, J=8.7 Hz, 3H), 8.02 (s, 1H), 8.60 (s, 1H), 10.29 (s, 1H), 11.87 (s, 1H).

### Example 26

### Preparation of 7-(4-(4-(benzylamino)-7H-pyrrolo[2,3-d]pyrimidin-6-yl)phenoxy)-N-hydroxyheptanamide (Compound 38)

### Step 26a. N-Benzyl-6-(4-methoxyphenyl)-7H-pyrrolo[2,3-d]pyrimidin-4-amine (Compound 411)

A mixture of compound 404 (2.59 g, 10 mmol) and phenylmethanamine (3.21 g, 30 mmol) in n-BuOH (30 mL) was stirred at 140° C. overnight. The mixture was cooled, filtered, washed with Et₂O to afford the product 411 as a yellow solid (3.0 g, 93%). LCMS: 331 [M+1]⁺.

### Step 26b. 4-(4-(Benzylamino)-7H-pyrrolo[2,3-d]pyrimidin-6-yl)phenol (Compound 412)

To a solution of compound 411 (2.5 g, 7.6 mmol) in dichloromethane (202 mL) was added a solution of BBr₃ (7.6 mL, 20.7 mmol) in dichloromethane (253 mL) at 0° C. under nitrogen over 1 hour. After the addition was completed, the mixture was allowed to warm to room temperature and stirred overnight. Then water (20 mL) was added to the mixture at −20° C. The mixture was warmed to room temperature, extracted with ethyl acetate (150 mL×3). The organic layer was washed with brine, dried, filtered, concentrated to give the product 412 as a yellow solid (1.43 g, 59%). LCMS: 317 [M+1]⁻.

### Step 26c. Ethyl 7-(4-(4-(benzylamino)-7H-pyrrolo[2,3-d]pyrimidin-6-yl)phenoxy)heptanoate (Compound 413-38)

To a mixture of compound 412 (300 mg, 0.9 mmol) and K₂CO₃ (248 mg, 1.8 mmol) in N,N-dimethylformamide (4.0 mL) was added ethyl 7-bromoheptanoate (213 mg, 0.9 mmol) and the resulting mixture was stirred at 70° C. for 20 h. DMF was removed under reduced pressure at 50° C. and was diluted with 30 mL of ethyl acetate. The organic layer was washed with water, dried over anhydrous Na₂SO₄, filtered and concentrated to give compound 413-38 (150 mg, 35%). LCMS: 473 [M+1]⁺.

### Step 26d. 7-(4-(4-(Benzylamino)-7H-pyrrolo[2,3-d]pyrimidin-6-yl)phenoxy)-N-hydroxyheptanamide (Compound 38)

To a flask containing compound 413-38 (100 mg, 0.21 mmol) was added the saturated solution of hydroxylamine in methanol (4.0 mL). The mixture was stirred at room temperature for 30 min. Then it was neutralized to pH7 using acetic acid. The mixture was concentrated under reduced pressure and the residue was washed with water, evaporated. The residue was purified by column chromatography to obtain the product as a white solid (40 mg, 42%). LCMS: 460 [M+1]⁺; ¹H NMR (DMSO-d₆): δ 1.30˜1.54 (m, 6H), 1.72 (t, J=8.1 Hz, 2H), 1.96 (t, J=7.2 Hz, 2H), 4.00 (t, J=6.0 Hz, 2H), 4.81 (d, J=4.5 Hz, 2H), 7.04 (d, J=9.0 Hz, 2H), 7.12 (s, 1H), 7.32-7.51 (m, 5H), 7.73 (d, J=8.4 Hz, 2H), 8.32 (s, 1H), 9.45 (s, 1H), 10.36 (s, 1H), 12.88 (s, 1H).

### Example 27

### Preparation of (R)—N-hydroxy-4-(4-(1-phenylethylamino)-7H-pyrrolo[2,3-d]pyrimidin-6-yl)benzamide (Compound 39)

A mixture of compound 110 (149 mg, 0.4 mmol) and NH₂OH/MeOH (3 mL, 5.31 mmol) was stirred at room temperature for 0.5 hour. The reaction mixture was neutralized with AcOH and concentrated. The residue was suspended in water and the resulting precipitate was isolated and dried to give crude product that was purified by preparative HPLC to give product 39 as a pale yellow solid (42 mg, 28%): LCMS: 374 [M+1]⁻; ¹H NMR: (DMSO-d₆): δ 1.53 (d, J=7.2 Hz, 3H), 5.50 (m, 1H), 7.22 (m, 2H), 7.31 (t, J=7.5 Hz, 2H), 7.42 (d, J=7.5 Hz, 2H), 7.84 (m, 5H), 8.08 (s, 1H), 9.06 (s, 1H), 11.23 (s, 1H), 12.13 (s, 1H).

### Example 28

### (R,E)-N-Hydroxy-3-(4-((4-(4-(1-phenylethylamino)-7H-pyrrolo[2,3-d]pyrimidin-6-yl)phenylamino)methyl)phenyl)acrylamide (Compound 42)

### Step 28a. (R,E)-Methyl3-(4-((4-(4-(1-phenylethylamino)-7H-pyrrolo[2,3-d]pyrimidin-6-yl)phenyl-amino)methyl)phenyl)acrylate (Compound 703-42)

A mixture of compound 506 (200 mg, 0.6 mmol) and 4-formylcinnamic acid (140 mg, 0.8 mmol) in 40 mL of methanol was refluxed for 1 hour. NaBH₃CN (50 mg, 0.8 mmol) was then added and the mixture was stirred for additional 2 hours. Thionyl chloride (0.5 mL) was added dropwise to the mixture and stirred for 3 hours. The reaction was monitored by TLC. Then the mixture was concentrated under reduced pressure. The residue was washed with water and filtered to obtain compound 703-42 as a yellow solid (208 mg, 68.8%). LCMS: 504 [M+1]⁺.

### Step 28b. (R,E)-N-Hydroxy-3-(4-((4-(4-(1-phenylethylamino)-7H-pyrrolo[2,3-d]pyrimidin-6-yl)phenylamino)methyl)phenyl)acrylamide (Compound 42)

A mixture of 703-42 (0.208 g, 0.41 mmol) and the saturated solution of hydroxylamine in methanol (1.77 mol/L, 10 mL) was stirred for 6 hours at room temperature. The mixture was adjusted to pH7˜8 with acetic acid. Solvent was removed and the residue was suspended in water, filtered and purified to give compound 42 as a yellow solid (0.060 g, 29.0%). m.p. 265.1˜294.1° C., LC-MS: 505 [M+1]⁻, ¹H NMR (300 MHz, DMSO-d₆): δ 1.49 (d, J=7.2 Hz, 3H), 4.32 (d, J=4.8 Hz, 2H), 5.45 (m, 1H), 6.30˜6.50 (m, 1H), 6.60 (d, J=8.4 Hz, 2H), 6.75 (s, 1h), 7.16 (t, J=7.2 Hz, J=6.6 Hz, 1H), 7.27 (t, J=7.5 Hz, 2H), 7.30˜7.60 (m, 10H), 7.96 (s, 1H), 8.95 (s, 1H), 10.68 (s, 1H), 11.64 (s, 1H).

### Example 29

### (R)—N-Hydroxy-4-((4-(4-(1-phenylethylamino)-7H-pyrrolo[2,3-d]pyrimidin-6-yl)phenylamino)methyl)benzamide (Compound 43)

### Step 29a. (R)-Methyl 4-((4-(4-(1-phenylethylamino)-7H-pyrrolo[2,3-d]pyrimidin-6-yl)phenylamino)methyl)benzoate (Compound 706-43)

To a suspension of compound 506 (200 mg, 0.6 mmol) and 4-formylbenzic acid (120 mg, 0.8 mmol) in methanol (40 mL) was refluxed for 1 hours. NaBH₃CN (50 mg, 0.8 mmol) was then added to the mixture and stirred for another 2 hours. Thionyl chloride (0.2 mL) was added dropwise, and the mixture was stirred for 3 hours. The reaction was monitored by TLC. Then the mixture was concentrated under reduced pressure and the residue was washed with water and filtered to obtain compound 706-43 as a yellow solid (267 mg, 93.0%). LCMS: 478 [M+1]⁺.

### Step 29b. (R)—N-Hydroxy-4-((4-(4-(1-phenylethylamino)-7H-pyrrolo[2,3-d]pyrimidin-6-yl)phenyl-amino)methyl)benzamide (Compound 43)

A mixture of 706-43 (0.267 g, 0.56 mmol) and the saturated solution of hydroxylamine in methanol (1.77 mol/L, 8 mL) was stirred for 6 hours at room temperature. The mixture was adjusted to pH=7˜8 with acetic acid and solvent was removed. The residue was diluted with water, filtered and purified to give compound 43 as a yellow solid (0.065 g, 24.3%): m.p. 169.3˜170.9° C., LC-MS: 479 [M+1]⁺, ¹H NMR (300 MHz, DMSO-d₆): δ 1.49 (d, J=7.2 Hz, 3H), 4.34 (d, J=5.4 Hz, 2H), 5.45 (m, 1H), 6.52 (t, J=6.0 Hz, 1H), 6.29 (d, J=8.7 Hz, 2H), 6.75 (s, 1H), 7.16 (t, J=7.5 Hz, 1H), 7.27 (t, J=7.2 Hz, 2H), 7.30-7.50 (m, 6H), 7.57 (d, J=7.8 Hz, 1H), 7.68 (d, J=8.1 Hz, 2H), 7.96 (s, 1H), 8.94 (s, 1H), 11.11 (s, 1H), 11.65 s, 1H).

### Example 30

### Preparation of (R)—N-hydroxy-4-((4-(4-(1-phenylethylamino)-7H-pyrrolo[2,3-d]pyrimidin-6-yl)benzylamino)methyl)benzamide (Compound 44)

### Step 30a. (R)-6-(4-(aminomethyl)phenyl)-N-(1-phenylethyl)-7H-pyrrolo[2,3-d]pyrimidin-4-amine (Compound 801)

A mixture of compound 112 (500 mg, 1.38 mmol) in ammonia (60 mL) was stirred and heated to 110° C. in a sealed system for 24 hours. The mixture was cooled to room temperature and resulting precipitate was isolated. The solution was diluted into the water, adjust the PH=2. The resulting precipitate was isolated and dried to yield title compound 801 as a grey solid (204 mg, 43%): LCMS: 344 [M+1]⁺.

### Step 30b. (R)-methyl4-((4-(4-(1-phenylethylamino)-7H-pyrrolo[2,3-d]pyrimidin-6-yl)benzylamino)methyl)benzoate (Compound 802-44)

A mixture of compound 801 (170 mg, 0.5 mmol), 4-formylbenzoic acid (75 mg, 0.5 mmol) and methanol (40 mL) was stirred and heated to reflux for 1 hour. NaBH3CN (50 mg, 0.75 mmol) was then added and the mixture was stirred under reflux for 2 hours. After that, sulfurous dichloride (90 mg, 0.75 mmol) was added and the mixture was stirred under reflux for additional 5 hours. The solvent was removed under reduced pressure and the residue was washed with water to get the crude product which was purified by column chromatography to yield title compound 802-44 as a grey solid (220 mg, 86%): LCMS: 492 [M+1]⁺.

### Step 30c. (R)—N-hydroxy-4-((4-(4-(1-phenylethylamino)-7H-pyrrolo[2,3-d]pyrimidin-6-yl)methyl)benzamide (Compound 44)

To compound 802-44 (150 mg, 0.3 mmol) was added freshly prepared hydroxylamine solution (1.7 mL, 3 mmol). The reaction mixture was stirred at 20° C. for 30 minutes and then warmed room temperature. The reaction process was monitored by TLC. The mixture was neutralized with acetic acid and the resulting mixture was concentrated under reduced pressure to yield a residue which was purified by preparation HPLC to give the title compound 44 as a grey solid (24 mg, 18%): LCMS: 493 [M+1]; ¹H NMR (DMSO-d₆) 1.50 (d, J=6.9 Hz, 3H), 3.68 (d, J=12.9 Hz, 4H), 5.48 (m, 1H), 7.05 (s, 1H), 7.17 (t, J=7.8 Hz, 1H), 7.37 (t, J=7.2 Hz, 2H), 7.70 (m, 6H), 8.03 (s, 1H), 8.93 (s, 1H), 11.12 (s, 1H), 11.94 (s, 1H).

### Example 31

### Preparation of (R,E)-N-hydroxy-3-(4-((4-(4-(1-phenylethylamino)-7H-pyrrolo[2,3-d]pyrimidin-6-yl)enzylamino)methyl)phenyl)acrylamide (Compound 45)

### Step 31a. (R,E)-methyl 3-(4-((4-(4-(1-phenylethylamino)-7H-pyrrolo[2,3-d]pyrimidin-6-yl)benzylamino)methyl)phenyl)acrylate (Compound 802-45)

The title compound 802-45 was prepared (153 mg, 48% yield) from 801 (211 mg, 0.62 mmol) and (E)-methyl 3-(4-formylphenyl)acrylate (118 mg, 0.62 mmol) using a procedure similar to that described for compound 802-44 (Example 30): LCMS: 517 [M+1]⁻.

### Step 31b. (R,E)-N-hydroxy-3-(4-((4-(4-(1-phenylethylamino)-7H-pyrrolo[2,3-d]pyrimidin-6-yl)benzylamino)methyl)phenyl)acrylamide (Compound 45)

The title compound 45 was prepared as a grey solid (40 mg, 26% yield) from compound 802-45 (154 mg, 0.30 mmol) and freshly prepared hydroxylamine in methanol (1.7 mL, 3.0 mmol) using a procedure similar to that described for compound 44 (Example 30): LCMS: 518 [M+1]⁻; ¹H NMR (DMSO-d⁶) δ 1.50 (d, J=7.2 Hz, 3H), 3.70 (d, J=6.9 Hz, 4H), 5.48 (t, J=9.6 Hz, 1H), 6.39 (d, J=15.9 Hz, 1H), 7.06 (s, 1H), 7.18 (t, 1H), 7.29 (m, 4H), 7.40 (d, J=7.5 Hz, 2H), 7.70 (d, J=8.1 Hz, 2H), 7.82 (d, J=8.4 Hz, 1H), 8.04 (s, 1H), 12.00 (s, 1H).

### Example 32

### Preparation of (R)—N-hydroxy-4-(2-(4-(4-(4-(1-phenylethylamino)-7H-pyrrolo[2,3-d]pyrimidin-6-yl)benzyl)piperazin-1-yl)ethoxy)butanamide (Compound 49)

### Step 32a. (R)-2-(4-(4-(4-(1-Phenylethylamino)-7H-pyrrolo[2,3-d]pyrimidin-6-yl)benzyl)piperazin-1-yl)ethanol (Compound 901)

A mixture of compound 112 (1.37 g, 3.78 mmol), 2-(piperazin-1-yl)ethanol (590 mg, 4.54 mmol) and potassium carbonate (1.07 g 7.56 mmol) in N,N-dimethylformamide (20 mL) was stirred at 50° C. overnight. The mixture was then cooled to room temperature and the solvent was removed under reduced pressure. The residue was washed with water, and dried to provide the title compound 901 as a brown solid (1.552 g, 90.2%): LCMS: 457 [M+1]⁺; ¹H NMR (DMSO-d₆); δ 1.50 (d, J=7.2, 3H), 2.34 (m, 8H), 3.44 (s, 4H), 4.36 (s, 1H), 5.48 (m, 1H), 7.06 (s, 1H), 7.15 (t, J=7.5, 1H), 7.29 (m, 6H), 7.73 (d, J=8.1, 2H), 7.79 (d, J=8.4, 1H), 8.04 (s, 1H), 12.00 (s, 1H).

### Step 32b. (R)-methyl 4-(2-(4-(4-(4-(1-phenylethylamino)-7H-pyrrolo[2,3-d]pyrimidin-6-yl)benzyl)piperazin-1-yl)ethoxy)butanoate (Compound 902-49)

To the solution of compound 901 (456 mg, 1 mmol) in DMF (20 mL) was added NaH (24 mg, 1 mmol) in ice bath temperature. The mixture was stirred at this temperature for 30 minuters, and then methyl 4-bromobutanoate (231 mg, 1.2 mmol) was added and the mixture was stirred at 50° C. overnight. The solvent was removed under reduced pressure to obtained the crude product which was purified by column chromatography to yield title compound 902-49 as a grey solid (225 mg, 40%): LCMS: 481 [M+1]⁺.

### Step 32c. (R)—N-hydroxy-4-(2-(4-((4-(1-phenylethylamino)-7H-pyrrolo[2,3-d]pyrimidin-6-yl)methyl)piperazin-1-yl)ethoxy)butanamide (Compound 49)

To compound 902-49 (147 mg, 0.377 mmol) was added freshly prepared hydroxylamine solution (4.3 mL, 7.5 mmol). The reaction was stirred at 0° C. for 30 minutes and then warmed to room temperature. The reaction process was monitored by TLC. The mixture was neutralized with acetic acid and the mixture was concentrated under reduce pressure to yield a residue which was purified by preparation HPLC to give the title compound 49 as a grey solid (70 mg, 48%): LCMS: 482 [M+1]⁺; ¹H NMR (DMSO-d₆) δ 1.50 (d, J=6.3 Hz, 3H), 1.76 (s, 4H), 2.39 (m, 10H), 3.48 (m, 4H), 4.17 (s, 2H), 4.35 (s, 1H), 5.50 (t, J=7.5, 1H), 6.76 (s, 1H), 7.24 (m, 1H), 7.32 (m, 2H), 7.32 (m, 2H), 7.42 (m, 6H), 7.82 (d, J=8.1, 2H), 8.10 (s, 1H), 8.61 (s, 1H), 10.27 (s, 1H).

### Example 33

### Preparation of (R)—N-hydroxy-5-(2-(4-(4-(4-(1-phenylethylamino)-7H-pyrrolo[2,3-d]pyrimidin-6-yl)benzyl)piperazin-1-yl)ethoxy)pentanamide (Compound 50)

### Step 33a. (R)-methyl 5-(2-(4-((4-(1-phenylethylamino)-7H-pyrrolo[2,3-d]pyrimidin-6-yl)methyl)piperazin-1-yl)ethoxy)pentanoate (Compound 902-50)

The title compound 902-50 was prepared (131 mg, 29% yield) from 901 (361 mg, 0.79 mmol) and 5-bromopentanoate (183 mg, 0.95 mmol) using a procedure similar to that described for compound 902-49 (Example 32): LCMS: 517 [M+1]⁺.

### Step 33b. (R)—N-hydroxy-5-(2-(4-(4-(4-(1-phenylethylamino)-7H-pyrrolo[2,3-d]pyrimidin-6-yl)benzyl)piperazin-1-yl)ethoxy)pentanamide (Compound 50)

The title compound 50 was prepared as a grey solid (90 mg, 69% yield) from compound 902-50 (130 mg, 0.23 mmol) and freshly prepared hydroxylamine in methanol (1.3 mL, 2.3 mmol) using a procedure similar to that described for compound 49 (Example 32): LCMS: 572 [M+1]; ¹H NMR (DMSO-d⁶) δ 1.23 (s, 2H), 1.50 (d, J=6.9 Hz, 3H), 1.78 (t, J=7.5 Hz, 2H), 2.41 (s, 8H), 3.30 (s, 2H), 3.48 (m, 3H), 4.17 (s, 2H), 4.35 (s, 1H), 5.50 (t, J=8.1 Hz, 1H), 6.75 (s, 1H), 7.18 (t, J=6.9 Hz, 1H), 7.29 (t, J=7.2 Hz, 1H), 7.42 (m, 5H), 7.81 (d, J=8.1 Hz, 1H), 8.09 (s, 1H), 8.60 (s, 1H), 10.21 (s, 1H).

### Example 34

### Preparation of (R)—N-hydroxy-6-(2-(4-(4-(4-(1-phenylethylamino)-7H-pyrrolo[2,3-d]pyrimidin-6-yl)benzyl)piperazin-1-yl)ethoxy)hexanamide (Compound 51)

### Step 34a. (R)-methyl 6-(2-(4-(4-(4-(1-phenylethylamino)-7H-pyrrolo[2,3-d]pyrimidin-6-yl)benzyl)piperazin-1-yl)ethoxy)hexanoate (Compound 902-51)

The title compound 902-51 was prepared as a grey solid (192 mg, 40% yield) from 901 (375 mg, 0.82 mmol) and methyl 6-bromohexanoate (204 mg, 0.98 mmol) using a procedure similar to that described for compound 902-49 (Example 32): LCMS: 585 [M+1]⁻.

### Step 34b. (R)—N-hydroxy-6-(2-(4-(4-(4-(1-phenylethylamino)-7H-pyrrolo[2,3-d]pyrimidin-6-yl)benzyl)piperazin-1-yl)ethoxy)hexanamide (Compound 51)

The title compound 51 was prepared as a grey solid (120 mg, 63% yield) from compound 902-51 (190 mg, 0.33 mmol) and freshly prepared hydroxylamine in methanol (1.9 mL, 3.3 mmol) using a procedure similar to that described for compound 49 (Example 32): LCMS: 586 [M+1]⁻; ¹H NMR (DMSO-d⁶) δ 1.00 (t, J=8.1 Hz, 2H), 1.31 (t, J=7.2 Hz, 2H), 1.50 (d, J=6.6 Hz, 3H), 1.78 (t, J=6.6 Hz, 2H), 2.37 (s, 8H), 3.48 (m, 3H), 4.17 (s, 2H), 4.35 (s, 1H), 5.50 (t, J=8.1 Hz, 1H), 6.75 (s, 1H), 7.18 (t, J=6.9 Hz, 1H), 7.29 (t, J=7.2 Hz, 1H), 7.42 (m, 5H), 7.81 (d, J=8.1 Hz, 1H), 8.04 (s, 1H), 8.60 (s, 1H), 10.22 (s, 1H).

### Example 35

### (R)—N-hydroxy-6-(1-(4-(4-(1-phenylethylamino)-7H-pyrrolo[2,3-d]pyrimidin-6-yl)benzyl)piperidin-4-ylamino)hexanamide (Compound 55)

### Step 35a. {6-[4-(1,4-Dioxa-8-aza-spiro[4.5]dec-8-ylmethyl)-phenyl]-7H-pyrrolo[2,3-d]pyrimidin-4-yl}-(1-phenyl-ethyl)-amine (Compound 1001)

To a solution of compound 112 (1.1 g, 3.0 mmol) in DMF (10 mL) was added 1,4-Dioxa-8-aza-spiro[4.5]decane (1.0 g, 7.0 mmol). The reaction was stirred at 10° C. for 1 hour. The solvent was evaporated under reduce pressure and the residue was washed with water, dried to obtain compound 1001 as a brown solid (1.2 g, 93% yield) LC-MS: 469 [M+1]⁺.

### Step 35b. 1-{4-[4-(1-Phenyl-ethylamino)-7H-pyrrolo[2,3-d]pyrimidin-6-yl]-benzyl}-piperidin-4-one (Compound 1002)

A solution of compound 1001 (1.2 g, 2.6 mmol) in THF (20 mL) and 20% H₂SO₄ (40 mL) was stirred at 50° C. for 4 hours. The mixture was neutralized by saturated NaHCO3. The precipitate was isolated and filtrated, dried to afford 1002 (1.0 g, 92% yield). LC-MS: 426 [M+1]⁻.

### Step 35c. 6-(1-{4-[4-(1-Phenyl-ethylamino)-7H-pyrrolo[2,3-d]pyrimidin-6-yl]-benzyl}-piperidin-4-ylamino)-hexanoic acid methyl ester (Compound 1003-55)

A solution of compound 1002 (130 mg, 0.31 mmol), 6-Amino-hexanoic acid methyl ester (46 mg, 0.31 mmol) and acetic acid (18.6 mg, 0.31 mmol) in 1,2-dichloro-ethane (10 mL) was treated with NaBH(OAc)₃ (92 mg, 1.2 mmol) and stirred at 25° C. over night. Saturated NaHCO₃ (10 mL) was added to the reaction mixture and the solvent was evaporated under reduce pressure to leave a residue. The residue was dissolved in THF and filtrated. The filtrated was concentrated and the crude product was purified by TLC to obtain compound 1003-55 as a brown solid (120 mg, 71% yield): LC-MS: 555 [M+1]⁺.

### Step 35d. 6-(1-{4-[4-(1-Phenyl-ethylamino)-7H-pyrrolo[2,3-d]pyrimidin-6-yl]-benzyl}-piperidin-4-ylamino)-hexanoic acid hydroxyamide (Compound 55)

To compound 1003-55 (60 mg, 0.1 μmol) was added freshly prepared hydroxylamine solution (1.0 mL, 1.8 mmol). The reaction mixture was sonicated for 40 minutes. The reaction process was monitored by TLC. After the completion of the reaction, the mixture was neutralized with acetic acid. The mixture was concentrated under reduce pressure and the residue was washed with water and dried to give the title compound 55 as a yellow solid (42 mg, 70%): LCMS: 556 [M+1]⁺; ¹H NMR (DMSO-d₆) δ 1.25 (m, 6H), 1.52 (d, J=6.6 Hz, 4H), 1.94 (m, 5H), 2.59 (t, J=7.5 Hz, 3H), 2.79 (d, J=11.1 Hz 2H), 3.4 (s, 2H), 5.48 (m, 1H), 7.07 (s, 1H), 7.18 (s, 1H), 7.26 (m, 1H), 7.31 (m, 5H), 7.42 (m, 2H), 7.72 (d, J=8.1 Hz, 1H), 7.72 (d, J=8.1 Hz, 1H), 7.80 (d, J=8.7 Hz, 1H), 8.04 (s, 1H), 11.98 (s, 1H).

### Example 36

### (R)—N-hydroxy-7-(1-(4-(4-(1-phenylethylamino)-7H-pyrrolo[2,3-d]pyrimidin-6-yl)benzyl)piperidin-4-ylamino)heptanamide (Compound 56)

### Step 36a. 7-(1-{4-[4-(1-Phenyl-ethylamino)-7H-pyrrolo[2,3-d]pyrimidin-6-yl]-benzyl}-piperidin-4-ylamino)-heptanoic acid methyl ester (Compound 1003-56)

The title compound 1003-56 was prepared as a yellow solid (60 mg, 36% yield) from 1002 and 7-Amino-heptanoic acid methyl ester (94 mg, 0.588 mmol) using a procedure similar to that described for compound 1003-55 (Example 35): LC-MS: 569 [M+1].

### Step 36b. 7-(1-{4-[4-(1-Phenyl-ethylamino)-7H-pyrrolo[2,3-d]pyrimidin-6-yl]-benzyl}-piperidin-4-ylamino)-heptanoic acid methyl ester (Compound 56)

The title compound 56 was prepared as a yellow solid (30 mg, 50% yield) from compound 1003-56 (60 mg, 0.11 mmol) using a procedure similar to that described for compound 55 (Example 35): LC-MS: 570 [M+1]⁺; ¹H NMR (DMSO-d₆) δ 1.25 (m, 6H), 1.48 (m, 5H), 1.53 (d, J=7.5 Hz, 4H), 1.98 (d, J=8.1 Hz, 4H), 2.7 (t, J=6.9 Hz, 3H), 2.53 (d, J=11.1 Hz 2H), 3.47 (s, 2H), 5.48 (m, 1H), 7.11 (s, 1H), 7.19 (m, 1H), 7.31 (m, 5H), 7.42 (m, 2H), 7.74 (d, J=8.4 Hz, 2H), 7.85 (d, J=8.1 Hz, 1H), 8.06 (s, 1H), 12.01 (s, 1H).

### Example 37

### Preparation of (R)—N-hydroxy-8-(1-(4-(4-(1-phenylethylamino)-7H-pyrrolo[2,3-d]pyrimidin-6-yl)benzyl)piperidin-4-ylamino)octanamide (Compound 57)

### Step 37a. 8-(1-{4-[4-(1-Phenyl-ethylamino)-7H-pyrrolo[2,3-d]pyrimidin-6-yl]-benzyl}-piperidin-4-ylamino)-octanoic acid methyl ester (Compound 1003-57)

The title compound 1003-57 was prepared as a yellow solid (70 mg, 36% yield) from 1002 and 8-Amino-octanoic acid methyl ester (102 mg, 0.588 mmol) using a procedure similar to that described for compound 1003-55 (Example 35): LC-MS: 583 [M+1]⁻.

### Step 37b. 8-(1-{4-[4-(1-Phenyl-ethylamino)-7H-pyrrolo[2,3-d]pyrimidin-6-yl]-benzyl}-piperidin-4-ylamino)-octanoic acid hydroxyamide (compound 57)

The title compound 57 was prepared as a yellow solid (40 mg, 67% yield) from compound 1003-57 (60 mg, 0.10 mmol) using a procedure similar to that described for compound 55 (Example 35): LCMS: 584 [M+1]⁺; ¹H NMR (DMSO-d₆) δ 1.23 (m, 6H), 1.42 (m, 7H), 1.73 (d, J=6.9 Hz, 4H), 1.96 (d, 4H), 2.64 (t, J=6.9 Hz, 3H), 2.80 (d, J=11.7 Hz, 2H), 3.45 (s, 2H), 5.49 (m, 1H), 7.08 (s, 1H), 7.20 (m, 1H), 7.31 (m, 5H), 7.42 (m, 2H), 7.72 (d, J=8.4 Hz, 2H), 7.80 (d, J=7.8 Hz, 1H), 8.04 (s, 1H), 11.99 (s, 1H).

### Example 38

### Preparation of (R)—N-hydroxy-6-(2-(4-(4-(1-phenylethylamino)-7H-pyrrolo[2,3-d]pyrimidin-6-yl)phenoxy)ethylamino)hexanamide (Compound 59)

### Step 38a. (R)-2-(4-(4-(1-phenylethylamino)-7H-pyrrolo[2,3-d]pyrimidin-6-yl)phenoxy)acetonitrile (Compound 1101)

A mixture of 406 (4.0 g, 12.12 mmol), K₂CO₃ (11.6 g, 90.60 mmol) and 2-chloroacetonitrile (0.91 g, 12.12 mmol) in acetone was stirred at 55° C. overnight. Then the reaction was filtered to remove K₂CO₃. The filtrate was evaporated to dry and the resulting solid was filtered, washed with methanol, and dried to get 1101 as a white solid (0.954 g, 21%): LCMS: 370 [M+1]⁺; ¹H NMR (DMSO-d₆): δ 1.54 (d, J=7.2 Hz, 3H), 5.22 (s, 2H), 5.50 (q, J=7.2 Hz, 1H), 7.02 (s, 1H), 7.16-7.20 (m, 3H), 7.28-7.33 (m, 2H), 7.43 (d, J=7.2 Hz, 1H), 7.76-7.81 (m, 3H), 8.04 (s, 1H), 11.96 (s, 1H).

### Step 38b. (R)-6-(4-(2-aminoethoxy)phenyl)-N-(1-phenylethyl)-7H-pyrrolo[2,3-d]pyrimidin-4-amine (Compound 1102)

To a 0° C. solution of 1101 (0.954 g, 2.58 mmol) in THF (120 mL) was added AlLiH₄ (0.294 g, 7.74 mmol) slowly. The mixture was warmed to room temperature for 20 min, then 1:1:3 (H₂O: 15% NaOH:H₂O) was added, filtrated and evaporated to obtain 1102 as white solid (0.788 g, 82.5%): LCMS: 374 [M+1]; ¹H NMR (DMSO-d₆): δ 1.53 (d, J=6.9 Hz, 3H), 2.88 (t, J=5.7 Hz, 2H), 3.96 (t, J=5.7 Hz, 2H), 5.50 (q, J=6.9 Hz, 1H), 6.96 (s, 1H), 7.02 (d, J=6.0 Hz, 2H), 7.17-7.22 (m, 1H), 7.30 (t, J=5.5 Hz, 2H), 7.43 (d, J=6.9 Hz, 2H), 7.71 (d, J=9.0 Hz, 3H), 8.04 (s, 1H), 11.93 (s, 1H).

### Step 38c. (R)-ethyl 6-(2-(4-(4-(1-phenylethylamino)-7H-pyrrolo[2,3-d]pyrimidin-6-yl)phenoxy)ethylamino)hexanoate (Compound 1103-59)

A mixture of ethyl 6-bromohexanoate (477 mg, 2.14 mmol) and 1102 (400 mg, 1.07 mmol) in DMF (5 mL) was stirred at 50° C. overnight. After reaction, solvent DMF was evaporated and 20 mL ethyl ether was added. The mixture was filtered, washed with ethyl ether and dried to obtain 1103-59 as a yellow solid (100 mg): LCMS: 516 [M+1].

### Step 38d. (R)—N-hydroxy-6-(2-(4-(4-(1-phenylethylamino)-7H-pyrrolo[2,3-d]pyrimidin-6-yl)phenoxy)ethylamino)hexanamide (Compound 59)

To a flask containing compound 1103-59 (100 mg, 0.19 mmol) was added to hydroxylamine methanol solution (4.0 mL). The mixture was stirred at room temperature for 30 min. Then it was adjusted to pH7 using acetic acid. The mixture was concentrated to give a residue which was purified by Pre-HPLC to yield compound 59 as a white solid (64 mg, 67%). LCMS: 503 [M+1]⁺; ¹H NMR (DMSO-d₆): δ 1.26-1.31 (m, 2H), 1.53 (d, J=6 Hz, 2H), 1.96 (t, J=3 Hz, 2H), 2.79 (t, J=6 Hz, 2H), 3.15-3.21 (m, 2H), 4.20 (s, 2H), 5.50 (q, J=6.9 Hz, 1H), 6.98 (s, 1H), 7.02 (d, J=6.0 Hz, 2H), 7.17-7.20 (m, 1H), 7.30 (t, J=5.5 Hz, 2H), 7.43 (d, J=6.9 Hz, 2H), 7.71 (d, J=9.0 Hz, 3H), 8.04 (s, 1H), 10.36 (s, 1H), 11.93 (s, 1H).

### Example 39

### Preparation of (R)—N-hydroxy-7-(2-(4-(4-(1-phenylethylamino)-7H-pyrrolo[2,3-d]pyrimidin-6-yl)phenoxy)ethylamino)heptanamide (Compound 60)

### Step 39a. (R)-methyl 7-(3-(4-(4-(1-phenylethylamino)-7H-pyrrolo[2,3-d]pyrimidin-6-yl)phenoxy)propylamino)heptanoate (Compound 1103-60)

The title compound 1103-60 was prepared (112 mg, 19% yield) from 1102 (400 mg, 1.07 mmol) and ethyl 7-bromoheptanoate (507 mg, 2.14 mmol) using a procedure similar to that described for compound 1103-59 (Example 38): LC-MS: 530 [M+1].

### Step 39b. (R)—N-hydroxy-7-(2-(4-(4-(1-phenylethylamino)-7H-pyrrolo[2,3-d]pyrimidin-6-yl)phenoxy)ethylamino)heptanamide (Compound 60)

The title compound 60 was prepared (73 mg, 69% yield) from compound 1103-60 (110 mg, 0.20 mmol) using a procedure similar to that described for compound 59 (Example 38): LCMS: 517 [M+1]; ¹H NMR (DMSO-d₆): δ 1.28 (s, 4H), 1.49-1.54 (m, 7H), 1.94 (t, J=6 Hz, 2H), 2.77 (t, J=6 Hz, 2H), 3.15-3.21 (m, 2H), 4.16 (s, 2H), 5.52 (q, J=6.9 Hz, 1H), 6.98 (s, 1H), 7.02 (d, J=6.0 Hz, 2H), 7.17-7.20 (m, 1H), 7.30 (t, J=5.5 Hz, 2H), 7.43 (d, J=6.9 Hz, 2H), 7.71 (d, J=9.0 Hz, 3H), 8.04 (s, 1H), 10.33 (s, 1H), 11.92 (s, 1H).

### Example 40

### Preparation of (R)—N-hydroxy-8-(2-(4-(4-(1-phenylethylamino)-7H-pyrrolo2,3-d]pyrimidin-6-yl)phenoxy)ethylamino)octanamide (Compound 61)

### Step 40a. (R)-methyl 8-(3-(4-(4-(1-phenylethylamino)-7H-pyrrolo[2,3-d]pyrimidin-6-yl)phenoxy)propylamino)octanoate (Compound 1103-61)

The title compound 1103-61 was prepared (95 mg, 16% yield) from 1102 (400 mg, 1.07 mmol) and 8-bromooctanoate (507 mg, 2.14 mmol) using a procedure similar to that described for compound 1103-59 (Example 38): LC-MS: 530 [M+1]⁺.

### Step 40b. (R)—N-hydroxy-8-(2-(4-(4-(1-phenylethylamino)-7H-pyrrolo[2,3-d]pyrimidin-6-yl)phenoxy)ethylamino)octanamide (compound 61)

The title compound 61 was prepared (55 mg, 59% yield) from compound 1103-61 (95 mg, 0.17 mmol) using a procedure similar to that described for compound 59 (Example 38): LCMS: 531 [M+1]⁺; ¹H NMR (DMSO-d₆): δ 1.26 (s, 6H), 1.42-1.53 (m, 7H), 1.90 (t, J=6 Hz, 2H), 2.81 (t, J=6 Hz, 2H), 3.14-3.18 (m, 2H), 4.17 (s, 2H), 5.50 (q, J=6.9 Hz, 1H), 6.95 (s, 1H), 7.04 (d, J=6.0 Hz, 2H), 7.15-7.20 (m, 1H), 7.30 (t, J=5.5 Hz, 2H), 7.43 (d, J=6.9 Hz, 2H), 7.71 (d, J=9.0 Hz, 3H), 8.04 (s, 1H), 10.32 (s, 1H), 11.92 (s, 1H).

### Example 41

### Preparation of (R)—N-hydroxy-6-(4-(4-(1-phenylethylamino)-7H-pyrrolo[2,3-d]pyrimidin-6-yl)phenylamino)hexanamide (Compound 66)

### Step 41a. (R)-ethyl 6-(4-(4-(1-phenylethylamino)-7H-pyrrolo[2,3-d]pyrimidin-6-yl)phenylamino)hexanoate (Compound 1201-66)

A mixture of compound 506 (500 mg, 1.52 mmol), ethyl 6-bromohexanoate (338.7 mg, 1.52 mmol) and DMF (15 mL) was stirred for 12 h at 50° C. The solvent was removed under high vacuum and the crude product purified by prep-HPLC to provide target compound 1201-66 (80 mg, 10%) as a yellow solid. LCMS: 472 [M+1].

### Step 41b. (R)—N-hydroxy-6-(4-(4-(1-phenylethylamino)-7H-pyrrolo[2,3-d]pyrimidin-6-yl)phenylamino)hexanamide (Compound 66)

A mixture of compound 1201-66 (80 mg, 0.17 mmol) and freshly prepared NH₂OH solution (1.77 M, 4 mL) was stirred for 15 min at room temperature. The mixture was adjusted to pH7.0 with AcOH and the solvent was removed. The solid was added with water, filtered and dried to provide compound 66 as a yellow solid (50 mg, 60%): m.p. 207˜217° C., LCMS: 473 [M+1]⁺; ¹H NMR (DMSO-d₆) δ 1.36 (m, 2H), 1.51˜1.53 (d, 7H, J=7.2 Hz), 1.96 (t, 2H, J=6.9 Hz), 3.03 (m, 2H), 5.43˜5.53 (m, 1H), 5.81 (t, 1H, J=5.4 Hz), 6.62 (d, 2H, J=8.4 Hz), 6.79 (s, 1H), 7.19 (m, 1H), 7.32 (m, 2H), 7.43 (m. 2H), 7.53 (d, 2H, J=7.2 Hz), 7.64 (d, 1H, J=7.8 Hz), 7.99 (s, 1H), 8.69 (s, 1H), 10.37 (s, 1H), 11.71 (s, 1H).

### Example 42

### Preparation of (R)—N-hydroxy-7-(4-(4-(1-phenylethylamino)-7H-pyrrolo[2,3-d]pyrimidin-6-yl)phenylamino)heptanamide (Compound 67)

### Step 42a. (R)-ethyl 7-(4-(4-(1-phenylethylamino)-7H-pyrrolo[2,3-d]pyrimidin-6-yl)phenylamino)heptanoate (Compound 1201-67)

The title compound 1201-67 was prepared as a yellow solid (105 mg, 14% yield) from 506 (500 mg, 1.52 mmol) and ethyl 7-bromoheptanoate (360 mg, 1.52 mmol) using a procedure similar to that described for compound 1201-66 (Example 41): LC-MS: 486 [M+1]⁺.

### Step 42b. (R)—N-hydroxy-7-(4-(4-(1-phenylethylamino)-7H-pyrrolo[2,3-d]pyrimidin-6-yl)phenylamino)heptanamide (Compound 67)

The title compound 67 was prepared as a yellow solid (85 mg, 86% yield) from compound 1201-67 (103 mg, 0.21 mmol) and freshly prepared hydroxylamine methanol solution (1.77 M, 5 mL) using a procedure similar to that described for compound 66 (Example 41): m.p. 125˜130° C., LCMS: 473 [M+1]; ¹H NMR (DMSO-d₆) δ 1.29 (m, 4H), 1.41˜1.51 (d, 7H, J=7.2 Hz), 1.96 (m, 2H), 3.03 (m, 2H), 5.43˜5.53 (m, 1H), 5.81 (t, 1H, J=5.4 Hz), 6.62 (d, 2H, J=8.4 Hz), 6.79 (s, 1H), 7.19 (m, 1H), 7.32 (m, 2H), 7.43 (m. 2H), 7.53 (d, 2H, J=7.2 Hz), 7.64 (d, 1H, J=7.8 Hz), 7.98 (s, 1H), 8.67 (s, 1H), 10.34 (s, 1H), 11.70 (s, 1H).

### Biological Assays:

As stated hereinbefore the derivatives defined in the present invention possess anti-proliferation activity. These properties may be assessed, for example, using one or more of the procedures set out below:

(a) An In Vitro Assay which Determines the Ability of a Test Compound to Inhibit a Receptor Tyrosine Kinase.

The ability of compounds to inhibit receptor kinase (EGFR, HER2/ErbB2, and VEGFR2) activity was assayed using HTScan™ Receptor Kinase Assay Kits (Cell Signaling Technologies, Danvers, Mass.). EGFR tyrosine kinase was obtained in partially purified form from GST-kinase fusion protein which was produced using a baculovirus expression system from a construct expressing human EGFR (His672-Ala1210) (GenBank Accession No. NM_{—}005228) with an amino-terminal GST tag. HER2/ErbB2 tyrosine kinase was produced using a baculovirus expression system from a construct containing a human HER2/ErbB2 c-DNA (GenBank Accession No. NM_{—}004448) fragment (Lys676-Val1255) amino-terminally fused to a GST tag. VEGFR2 tyrosine kinase was produced using a baculovirus expression system from a construct containing a human VEGFR2 cDNA kinase domain (Asp805-Val1356) (GenBank accession No. AF035121) fragment amino-terminally fused to a GST-HIS6-Thrombin cleavage site. The proteins were purified by one-step affinity chromatography using glutathione-agarose. An anti-phosphotyrosine monoclonal antibody, P-Tyr-100, was used to detect phosphorylation of biotinylated substrate peptides (EGFR, Biotin-PTP1B (Tyr66); HER2/ErbB2, Biotinylated FLT3 (Tyr589); VEGFR2, Biotin-Gastrin Precursor (Tyr87).). Enzymatic activity was tested in 60 mM HEPES, 5 mM MgCl₂ 5 mM MnCl₂ 200 μM ATP, 1.25 mM DTT, 3 μM Na₃VO₄, 1.5 mM peptide, and 50 ng EGF Recpetor Kinase. Bound antibody was detected using the DELFIA system (PerkinElmer, Wellesley, Mass.) consisting of DELFIA® Europium-labeled Anti-mouse IgG (PerkinElmer, #AD0124), DELFIA® Enhancement Solution (PerkinElmer, #1244-105), and a DELFIA® Streptavidin coated, 96-well Plate (PerkinElmer, AAAND-0005). Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm.

Test compounds were dissolved in dimethylsulphoxide (DMSO) to give a 20 mM working stock concentration. Each assay was setup as follows: Added 100 μl of 10 mM ATP to 1.25 ml 6 mM substrate peptide. Diluted the mixture with dH₂0 to 2.5 ml to make 2×ATP/substrate cocktail ([ATP]=400 mM, [substrate]=3 mM). Immediately transfer enzyme from −80° C. to ice. Allowed enzyme to thaw on ice. Microcentrifuged briefly at 4° C. to bring liquid to the bottom of the vial. Returned immediately to ice. Added 10 μl of DTT (1.25 mM) to 2.5 ml of 4×HTScan™ Tyrosine Kinase Buffer (240 mM HEPES pH 7.5, 20 mM MgCl₂, 20 mM MnCl, 12 mM NaVO₃) to make DTT/Kinase buffer. Transfer 1.25 ml of DTT/Kinase buffer to enzyme tube to make 4× reaction cocktail ([enzyme]=4 ng/μL in 4× reaction cocktail). Incubated 12.5 μl of the 4× reaction cocktail with 12.5 μl/well of prediluted compound of interest (usually around 10 μM) for 5 minutes at room temperature. Added 25 μl of 2×ATP/substrate cocktail to 25 μg/well preincubated reaction cocktail/compound. Incubated reaction plate at room temperature for 30 minutes. Added 50 μl/well Stop Buffer (50 mM EDTA, pH 8) to stop the reaction. Transferred 25 μl of each reaction and 75 μl dH₂O/well to a 96-well streptavidin-coated plate and incubated at room temperature for 60 minutes. Washed three times with 200 μl/well PBS/T (PBS, 0.05% Tween-20). Diluted primary antibody, Phospho-Tyrosine mAb (P-Tyr-100), 1:1000 in PBS/T with 1% bovine serum albumin (BSA). Added 100 μl/well primary antibody. Incubated at room temperature for 60 minutes. Washed three times with 200 μl/well PBS/T. Diluted Europium labeled anti-mouse IgG 1:500 in PBS/T with 1% BSA. Added 100 μl/well diluted antibody. Incubated at room temperature for 30 minutes. Washed five times with 200 μl/well PBS/T. Added 100 μl/well DELFIA® Enhancement Solution. Incubated at room temperature for 5 minutes. Detected 615 nm fluorescence emission with appropriate Time-Resolved Plate Reader.

(b) An In Vitro Assay which Determines the Ability of a Test Compound to Inhibit the EGF-Stimulated EGFR Phosphorylation.

Allowed A431 cell growth in a T75 flask using standard tissue culture procedures until cells reach near confluency (˜1.5×10⁷) cells; D-MEM, 10% FBS). Under sterile conditions dispensed 100 μl of the cell suspension per well in 96-well microplates (x cells plated per well). Incubated cells and monitor cell density until confluency is achieved with well-to-well consistency; approximately three days. Removed complete media from plate wells by aspiration or manual displacement. Replaced media with 50 μl of pre-warmed serum free media per well and incubated 4 to 16 hours. Made two fold serial dilutions of inhibitor using pre-warmed D-MEM so that the final concentration of inhibitor range from 10 μM to 90 μM. Removed media in A431 cell plate. Added 100 μl of serial diluted inhibitor into cells and incubate 1 to 2 hours. Removed inhibitor from plate wells by aspiration or manual displacement. Added either serum free media for resting cells (mock) or serum free media with 100 ng/ml EGF. Used 100 μl of resting/activation media per well. Allowed incubation at 37° C. for 7.5 minutes. Removed activation or stimulation media manually or by aspiration. Immediately fixed cells with 4% formaldehyde in 1×PBS. Allowed incubation on bench top for 20 minutes at RT with no shaking. Washed five times with 1×PBS containing 0.1% Triton X-100 for 5 minutes per Wash. Removed Fixing Solution. Using a multi-channel pipettor, added 200 μl of Triton Washing Solution (1×PBS+0.1% Triton X-100). Allowed wash to shake on a rotator for 5 minutes at room temperature. Repeated washing steps 4 more times after removing wash manually. Using a multi-channel pipettor, blocked cells/wells by adding 100 μl of LI-COR Odyssey Blocking Buffer to each well. Allowed blocking for 90 minutes at RT with moderate shaking on a rotator. Added the two primary antibodies into a tube containing Odyssey Blocking Buffer. Mixed the primary antibody solution well before addition to wells (Phospho-EGFR Tyr1045, (Rabbit; 1:100 dilution; Cell Signaling Technology, 2237; Total EGFR, Mouse; 1:500 dilution; Biosource International, AHR5062). Removed blocking buffer from the blocking step and added 40 μl of the desired primary antibody or antibodies in Odyssey Blocking Buffer to cover the bottom of each well. Added 100 μl of Odyssey Blocking Buffer only to control wells. Incubated with primary antibody overnight with gentle shaking at RT. Washed the plate five times with 1×PBS+0.1% Tween-20 for 5 minutes at RT with gentle shaking, using a generous amount of buffer. Using a multi-channel pipettor added 200 μl of Tween Washing Solution. Allowed wash to shake on a rotator for 5 minutes at RT. Repeated washing steps 4 more times. Diluted the fluorescently labeled secondary antibody in Odyssey Blocking Buffer (Goat anti-mouse IRDye™ 680 (1:200 dilution; LI-COR Cat.#926-32220) Goat anti-rabbit IRDye™ 800CW (1:800 dilution; LI-COR Cat.#926-32211). Mixed the antibody solutions well and added 40 μl of the secondary antibody solution to each well. Incubated for 60 minutes with gentle shaking at RT. Protected plate from light during incubation. Washed the plate five times with 1×PBS+0.1% Tween-20 for 5 minutes at RT with gentle shaking, using a generous amount of buffer. Using a multi-channel pipettor added 200 μl of Tween Washing Solution. Allowed wash to shake on a rotator for 5 minutes at RT. Repeated washing steps 4 more times. After final wash, removed wash solution completely from wells. Turned the plate upside down and tap or blot gently on paper towels to remove traces of wash buffer. Scanned the plate with detection in both the 700 and 800 channels using the Odyssey Infrared Imaging System (700 nm detection for IRDye™ 680 antibody and 800 nm detection for IRDye™ 800CW antibody). Determined the ratio of total to phosphorylated protein (700/800) using Odyssey software and plot the results in Graphpad Prism (V4.0a). Data were plotted using GraphPad Prism (v4.0a) and IC50's calculated using a sigmoidal dose response curve fitting algorithm.

(c) An In Vitro Assay which Determines the Ability of a Test Compound to Inhibit HDAC Enzymatic Activity.

HDAC inhibitors were screened using an HDAC fluorimetric assay kit (AK-500, Biomol, Plymouth Meeting, Pa.). Test compounds were dissolved in dimethylsulphoxide (DMSO) to give a 20 mM working stock concentration. Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm. Each assay was setup as follows: Defrosted all kit components and kept on ice until use. Diluted HeLa nuclear extract 1:29 in Assay Buffer (50 mM Tris/Cl, pH 8.0, 137 mM NaCl, 2.7 mM KCl, 1 mM MgCl2). Prepared dilutions of Trichostatin A (TSA, positive control) and tested compounds in assay buffer (5× of final concentration). Diluted Fluor de Lys™ Substrate in assay buffer to 100 uM (50 fold=2× final). Diluted Fluor de Lys™ developer concentrate 20-fold (e.g. 50 μl plus 950 μl Assay Buffer) in cold assay buffer. Second, diluted the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Added Assay buffer, diluted trichostatin A or test inhibitor to appropriate wells of the microtiter plate. Added diluted HeLa extract or other HDAC sample to all wells except for negative controls. Allowed diluted Fluor de Lys™ Substrate and the samples in the microtiter plate to equilibrate to assay temperature (e.g. 25 or 37° C. Initiated HDAC reactions by adding diluted substrate (25 μl) to each well and mixing thoroughly. Allowed HDAC reactions to proceed for 1 hour and then stopped them by addition of Fluor de Lys™ Developer (50 μl). Incubated plate at room temperature (25° C.) for 10-15 min. Read samples in a microtiter-plate reading fluorimeter capable of excitation at a wavelength in the range 350-380 nm and detection of emitted light in the range 440-460 nm.

The following TABLE 4-B lists compounds representative of the invention and their activity in HDAC, HER2/Erb2, VEGFR2 and EGFR assays. In these assays, the following grading was used: I≧10 μM, 10 μM>II>1 μM, 1 μM>III>0.1 μM, and IV≦0.1 μM for IC₅₀.

TABLE 4-B

| | | | HER2/ | |
|---|---|---|---|---|
| Compound No. | HDAC | EGFR | ErbB2 | VEGFR2 |
| 1 | II | II | II | N/A |
| 2 | I | IV | III | III |
| 11 | I | IV | IV | IV |
| 12 | I | IV | IV | III |
| 13 | II | IV | IV | III |
| 14 | II | IV | III | III |
| 15 | III | IV | III | III |
| 16 | III | IV | IV | III |
| 17 | IV | IV | IV | II |
| 19 | III | IV | III | III |
| 20 | III | IV | III | III |
| 21 | IV | IV | III | II |
| 22 | IV | IV | III | IV |
| 24 | I | IV | III | III |
| 25 | III | IV | III | III |
| 26 | IV | IV | III | III |
| 27 | IV | IV | III | III |
| 28 | I | III | III | III |
| 29 | IV | IV | III | III |
| 30 | III | IV | III | III |
| 31 | IV | IV | III | III |
| 32 | III | IV | III | III |
| 33 | IV | IV | III | III |
| 34 | III | III | III | III |
| 35 | III | IV | III | III |
| 36 | III | III | III | III |
| 37 | IV | III | III | II |
| 38 | IV | III | II | II |
| 39 | III | IV | IV | III |
| 40 | III | IV | III | II |
| 41 | III | IV | III | III |
| 42 | III | IV | II | II |
| 43 | III | IV | III | II |
| 44 | III | IV | III | III |
| 45 | IV | I | I | I |
| 46 | III | | | |
| 47 | II | | | |
| 49 | I | | | |
| 50 | II | | | |
| 51 | IV | | | |
| 55 | II | | | |
| 56 | II | | | |
| 57 | III | | IV | |
| 58 | III | | III | II |
| 59 | III | | | |
| 60 | III | IV | IV | IV |
| 61 | III | | IV | |
| 62 | II | | | |
| 63 | III | | | |
| 64 | III | | | |
| 65 | IV | | | |
| 66 | IV | IV | IV | IV |
| 67 | IV | IV | IV | III |

TABLE 5-A
SECTION 5:

| | |
|---|---|
| | (VIII) |
| 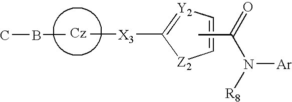 | |
| | (IX) |
| 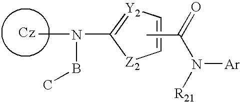 | |

| | |
|---|---|
| Compound # | Structure |

| | |
|---|---|
| 1 | 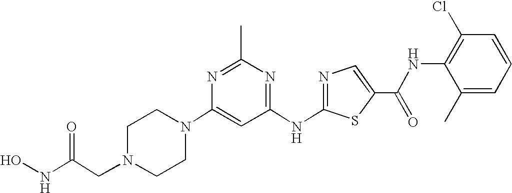 |
| 2 | 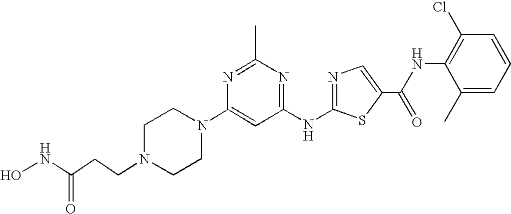 |
| 3 | 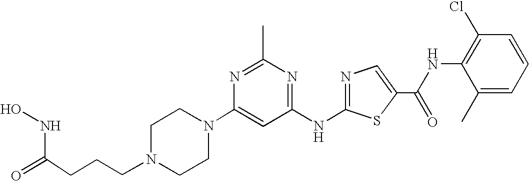 |
| 4 | 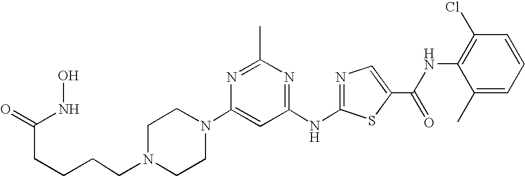 |
| 5 | 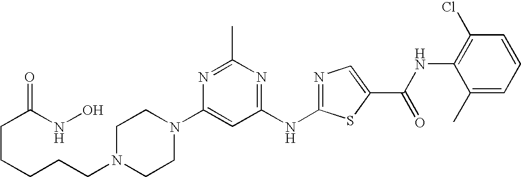 |
| 6 | 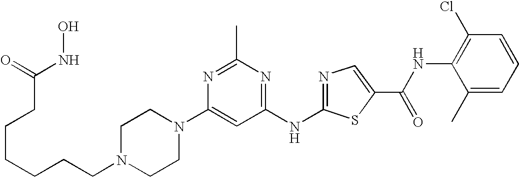 |
| 7 | 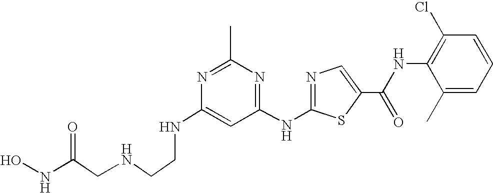 |
| 8 | 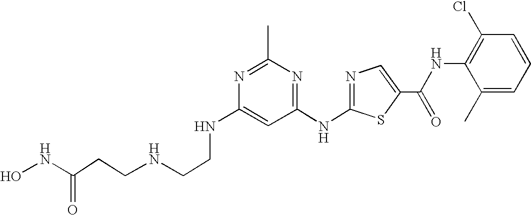 |
| 9 | 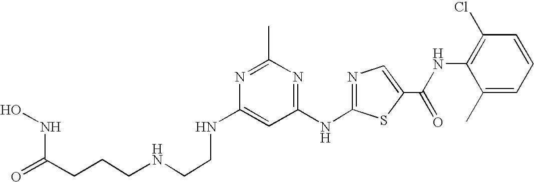 |
| 10 | 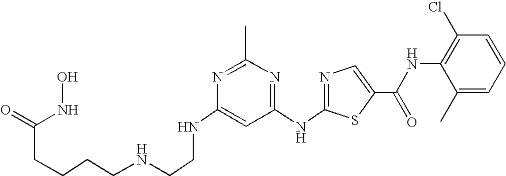 |
| 11 | 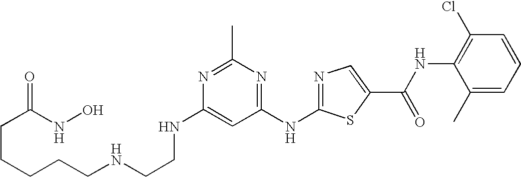 |
| 12 | 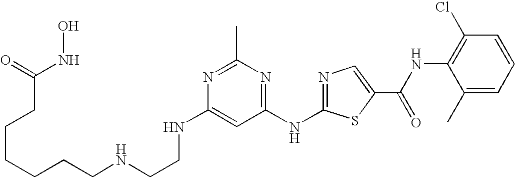 |
| 13 | 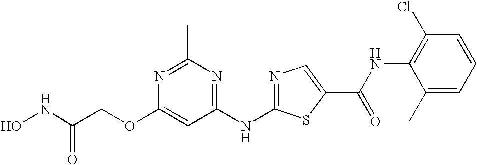 |
| 14 | 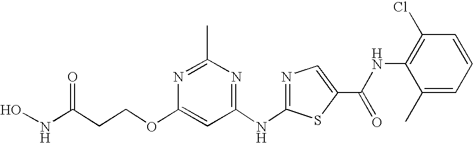 |
| 15 | 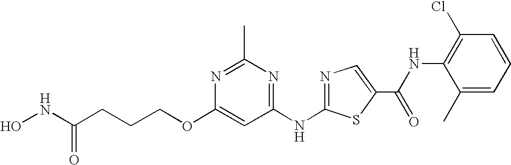 |
| 16 | 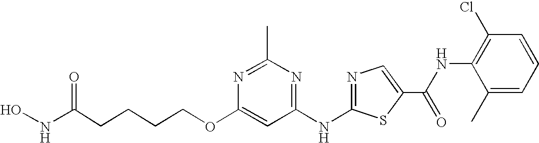 |
| 17 | 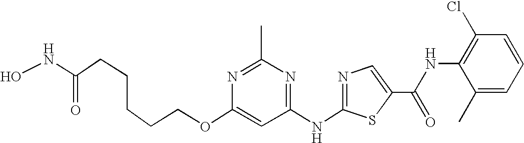 |
| 18 | 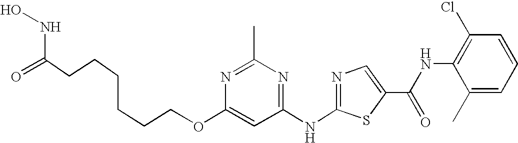 |
| 19 | 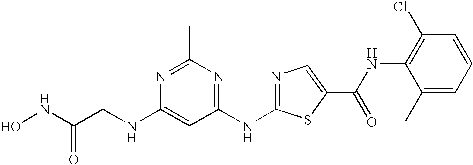 |
| 20 | 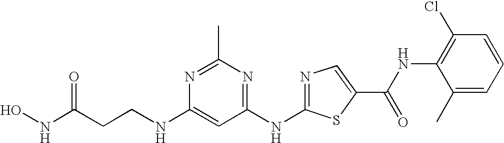 |
| 21 | 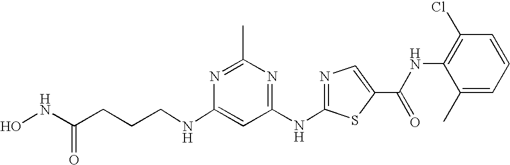 |
| 22 | 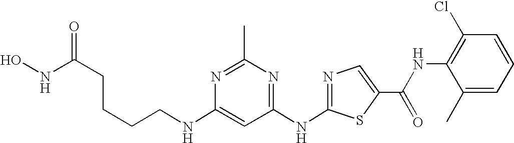 |
| 23 | 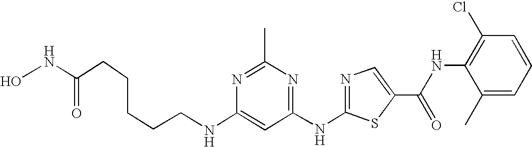 |
| 24 | 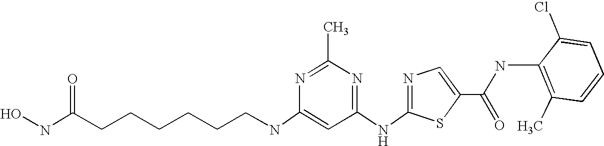 |
| 25 | 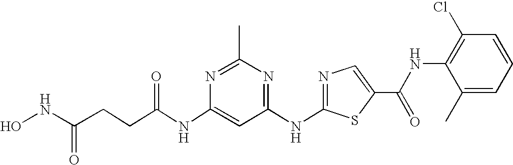 |
| 26 | 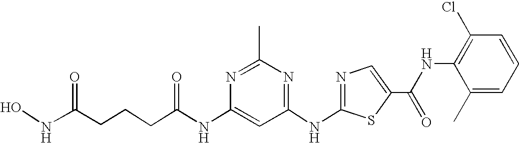 |
| 27 | 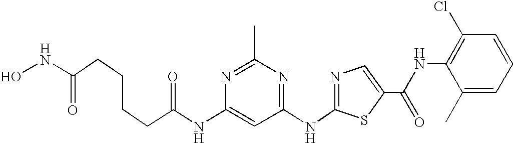 |
| 28 | 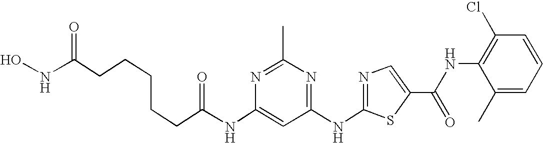 |
| 29 | 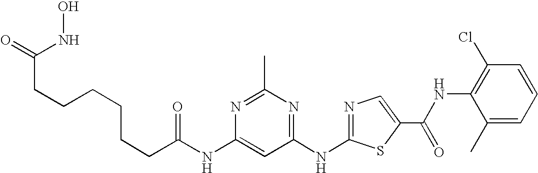 |
| 30 | 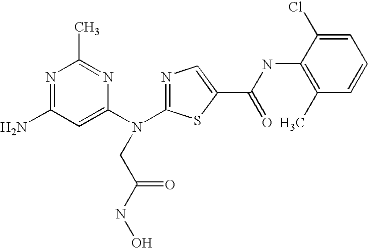 |
| 31 | 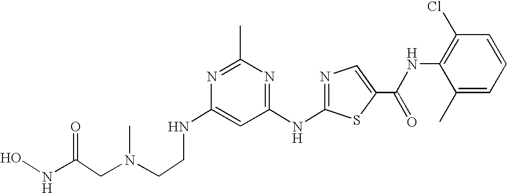 |
| 32 | 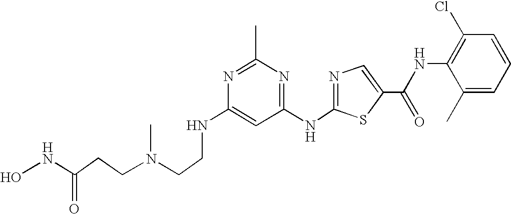 |
| 33 | 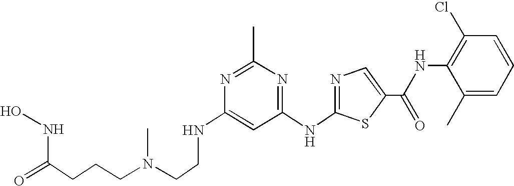 |
| 34 | 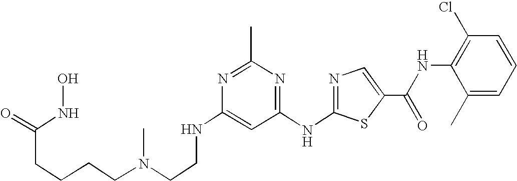 |
| 35 | 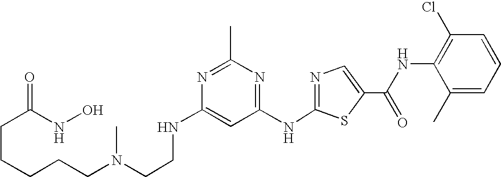 |
| 36 | 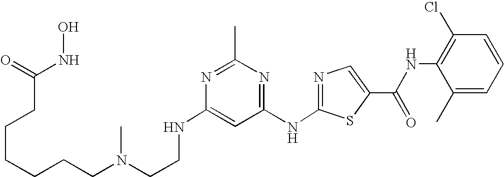 |
| 37 | 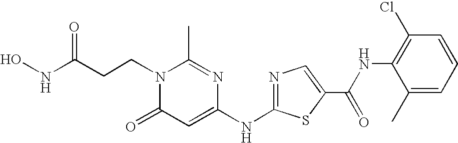 |
| 38 | 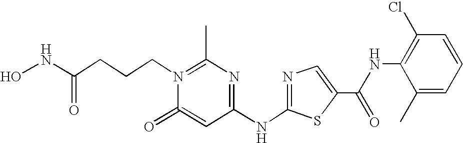 |
| 39 | 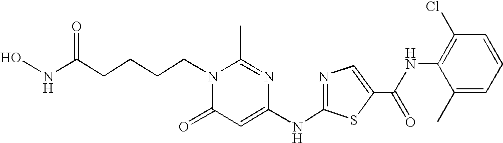 |
| 40 | 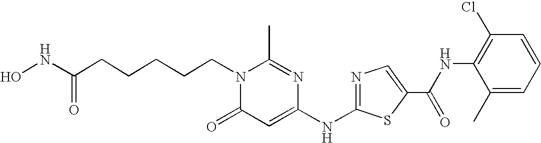 |
| 41 | 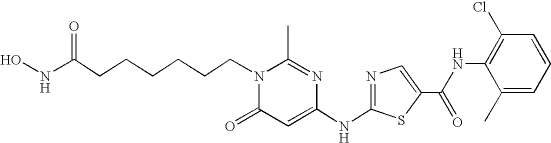 |
| 42 | 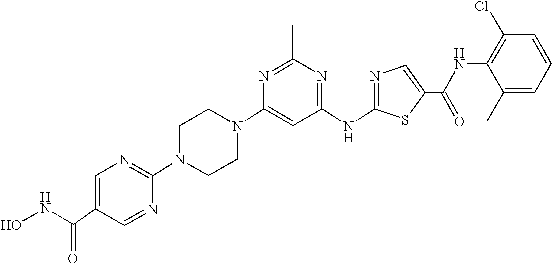 |
| 43 | 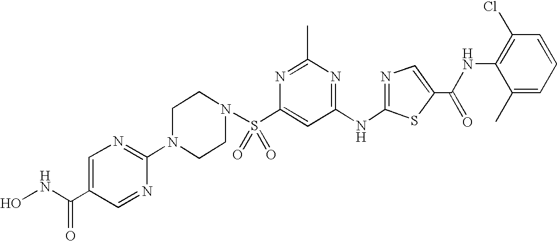 |

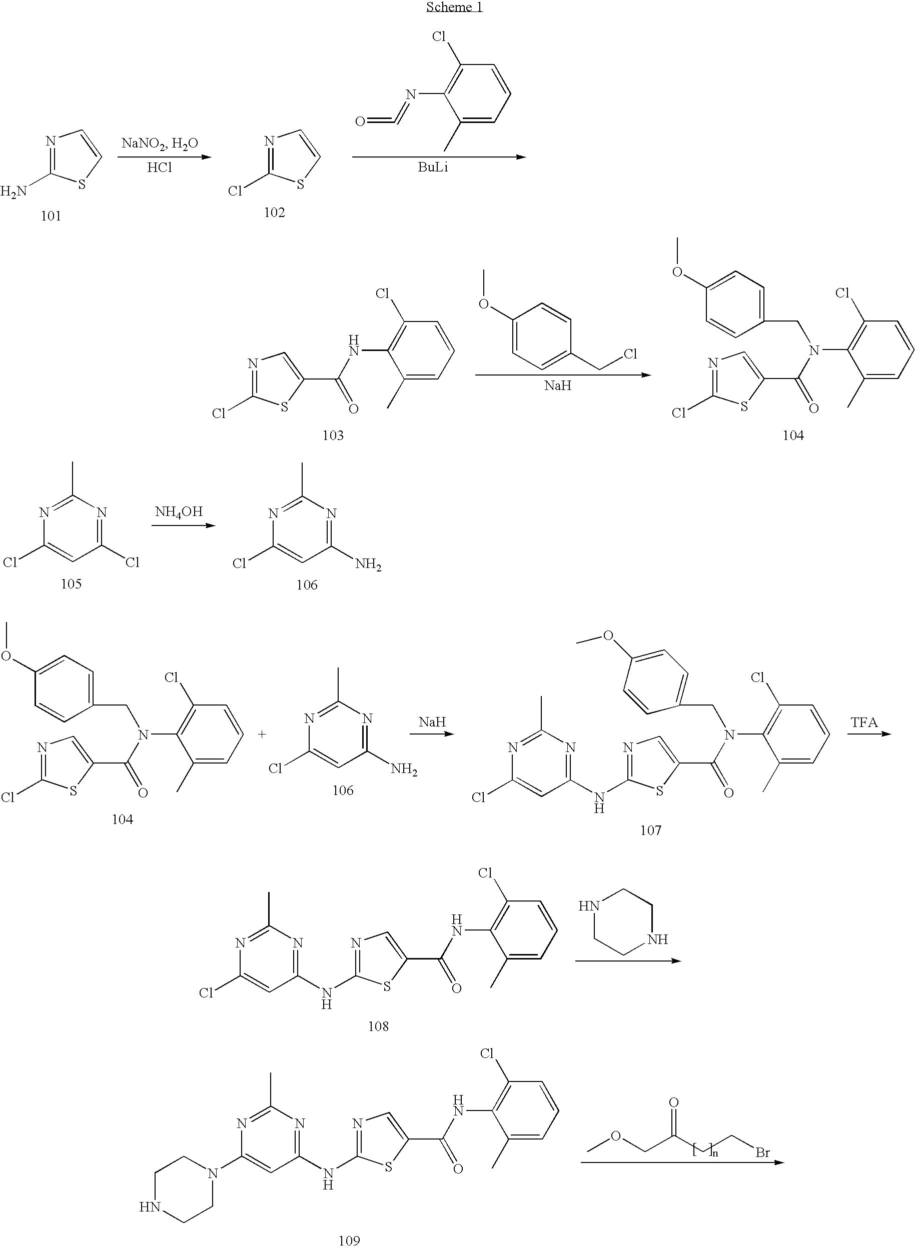

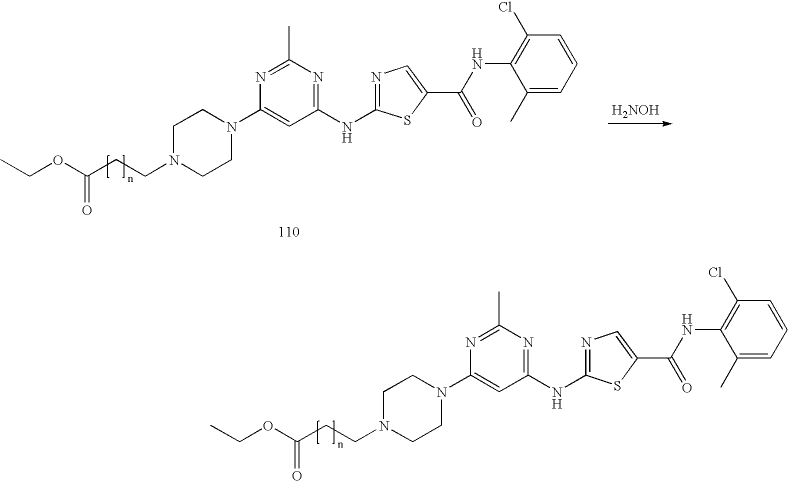

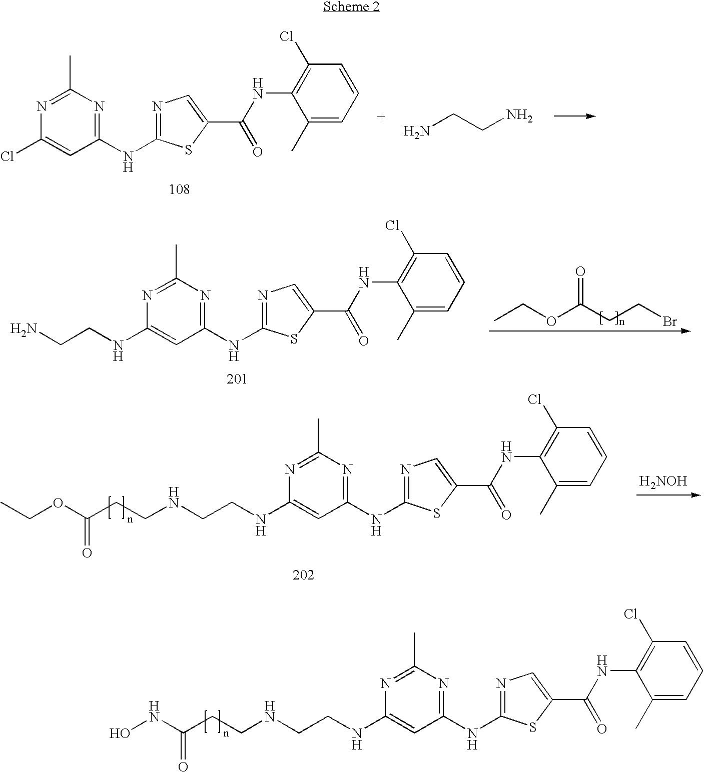

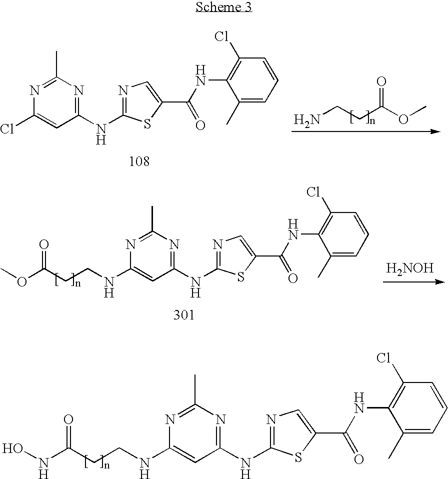

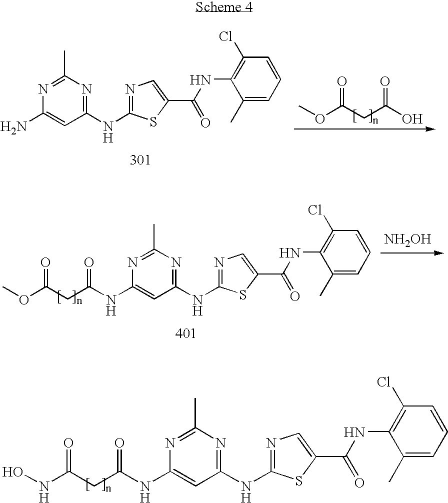

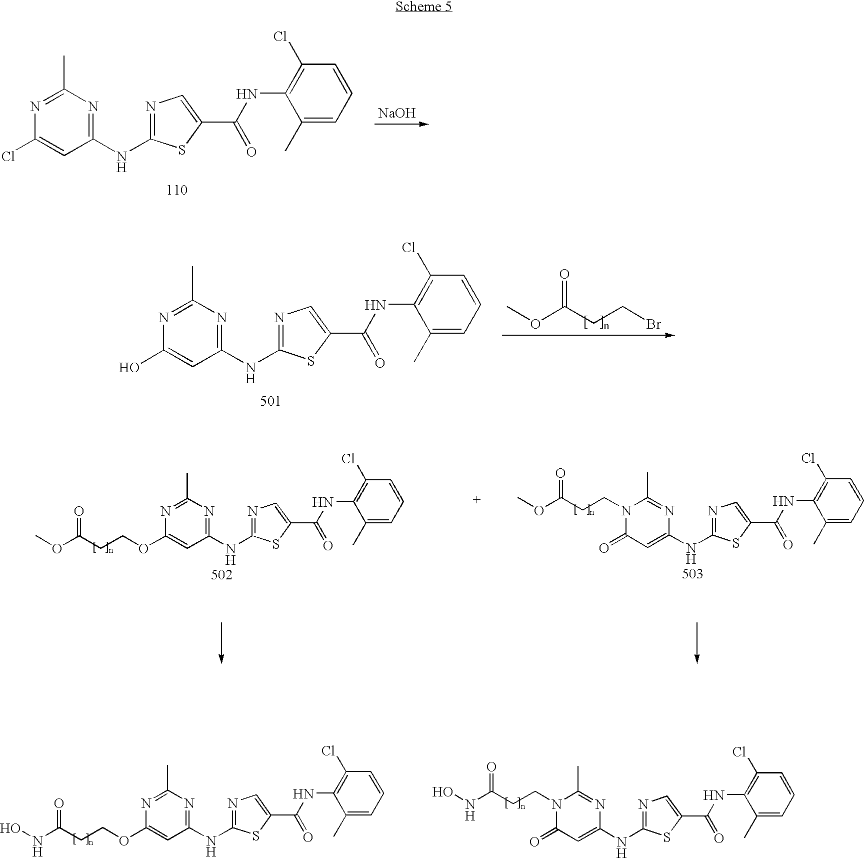

### Example 1

### Preparation of N-(2-chloro-6-methylphenyl)-2-(6-(4-(2-(hydroxyamino)-2-oxoethyl)piperazin-1-yl)-2-methylpyrimidin-4-ylamino)thiazole-5-carboxamide (Compound 1)

### Step 1a. 2-Chlorothiazole (Compound 102)

A solution of 2-aminothiazole (101) (20.0 g, 200.0 mmol) in saturated aqueous NaCl (20 mL) and HCl (60 mL) was maintained in a room-temperature bath. It was then treated with NaNO₂ (250 mmol) in H₂O (50 mL) and concentrated HCl (20 mL) dropwise simultaneously. The reaction was stirred at room temperature for 1 hour, extracted with ether and concentrated at 1 atm. The product was obtained by distilled under vacuum to give 102 as a pale yellow liquid (10.7 g, 45%): ¹H NMR (CDCl₃) δ 7.24 (d, J=3.6 Hz, 1H), 7.57 (d, J=3.3 Hz, 1H).

### Step 1b. 2-Chloro-N-(2-chloro-6-methylphenyl)thiazole-5-carboxamide (Compound 103)

A solution of 2-chlorothiazole (102) (480 mg, 4.0 mmol) in THF (10 mL) was cooled to −78° C. and treated dropwise with 2.5 M n-butyllithium in hexanes (1.68 mL, 4.2 mmol) over a period of 20 minutes while keeping the temperature below −75° C. After the addition was complete, the mixture was stirred at −78° C. for 15 minutes and then treated with a solution of 2-chloro-6-methylphenylisocyanate (4.4 mmol) in THF (5 mL). The mixture was stirred at −78° C. for 2 hours, quenched with saturated aqueous NH₄Cl, warmed to room temperature and partitioned between EtOAc and H₂O. The EtOAc phase was separated, washed with brine, dried (Na₂SO₄) and concentrated under vacuum to afford a yellow solid. The crude product was purified by column chromatography to give compound 103 as a pale yellow solid (0.95 g, 83%). LCMS: 286 [M+1]. ¹H NMR (DMSO-d₆): δ 2.22 (s, 3H), 7.29 (m, 2H), 7.41 (dd, J=6.3, J=3.0 Hz, 1H), 8.45 (s, 1H), 10.40 (s, 1H).

### Step 1c. 2-Chloro-N-(2-chloro-6-methylphenyl)-N-(4-methoxybenzyl)thiazole-5-carboxamide (Compound 104)

A solution of 2-chloro-N-(2-chloro-6-methylphenyl)thiazole (103) (0.57 g, 2.0 mmol) in DMF (5 mL) was treated with 60% NaH (2.4 mmol) and stirred at room temperature for 30 minutes. The mixture was treated with 4-methoxybenzyl chloride (0.38 g, 2.4 mmol) and tetrabutylammonium iodide (0.15 mg, 0.40 mmol), and then stirred at room temperature for 16 h. The mixture was partitioned between H₂O and EtOAc and then the EtOAc phase was separated, washed with brine, dried (Na₂SO₄) and concentrated under vacuum. The crude product was purified by column chromatography to give compound 104 as a yellow solid (0.50 g, 62%). LCMS: 429 [M+Na]⁻. ¹H NMR (DMSO-d₆): δ 1.73 (s, 3H), 3.60 (s, 3H), 4.48 (d, J=13.8 Hz, 1H), 5.09 (d, J=14.1 Hz, 1H), 6.79 (d, J=8.4 Hz, 2H), 7.11 (d, J=8.7 Hz, 2H), 7.29 (d, J=6.6 Hz, 1H), 7.44 (m, 3H).

### Step 1d. 6-Chloro-2-methylpyrimidin-4-amine (Compound 106)

A solution of 4,6-dichloro-2-methylpyrimidin (105) (20.0 g, 120 mmol) was placed in a tube with ammonium hydroxide (50 mL). The tube was sealed and heated at 125-128° C. for 10 hours. After cooling, the tube was opened and the reaction mixture (coarse, white crystals) was filtrated, giving the product compound 106 as a white solid (12.4 g, 70%). LCMS: 144 [M+1]^{+ 1}H NMR (DMSO-d₆): δ 2.27 (s, 3H), 6.24 (s, 1H), 7.08 (s, 2H).

### Step 1e. 2-((6-Chloro-2-methylpyrimidin-4-yl)methyl)-N-(2-chloro-6-methylphenyl)-N-(4-methoxybenzyl)thiazole-5-carboxamide (Compound 107)

4-Amino-6-chloro-2-methylpyrimidine (106) (14.0 mg, 0.10 mmol) was added in portions to a suspension of NaH (60% dispersion, 0.30 mmol) in THF (30 mL) at 0° C. for 30 minutes and then treated with compound 104 (41.0 mg, 0.10 mmol) in portions. The resulting mixture was at reflux for 4 hours, cooled to room temperature and diluted with H₂O (10 mL). The mixture was acidified with 1 N HCl (5 mL) and extracted with EtOAc (3×10 mL). The organic layer was dried (Na₂SO₄) and evaporated. The crude product was purified by column chromatography to give compound 107 as a pale yellow solid (41 mg, 80%): ¹H NMR (DMSO-d₆): δ 1.72 (s, 3H), 2.45 (s, 3H), 3.71 (s, 3H), 4.40 (d, J=14.1 Hz, 1H) 5.19 (d, J=13.8 Hz, 1H), 6.81 (m, 3H), 7.14 (d, J=8.4 Hz, 2H), 7.29 (d, J=7.5 Hz, 1H), 7.43 (t, J=7.8, Hz, 1H), 7.47 (s, 1H), 7.53 (d, J=6.9 Hz, 1H), 12.07 (ds, 1H).

### Step 1f. 2-(6-Chloro-2-methylpyrimidin-4-ylamino)-N-(2-chloro-6-methylphenyl)thiazole-5-carboxamide (Compound 108)

A solution of compound 107 (10.0 g, 19.5 mmol) dissolved in 50% TFA in CH₂Cl₂ (50 mL) was treated with triflic acid (10.0 g, 67.5 mmol). The reaction mixture was stirred at room temperature for 24 hours. The mixture was poured into crushed ice (150 g). The resulting solid was collected by filtration to obtain compound 108 as a yellow solid (5.6 g, 87%). ¹H NMR (DMSO-d₆): δ 2.21 (s, 3H), 2.39 (s, 3H), 6.07 (m, 1H), 7.26 (m, 2H), 7.37 (d, J=6.6 Hz, 1H), 8.20 (s, 1H), 9.85 (s, 1H), 11.47 (s, 1H).

### Step 1g. N-(2-Chloro-6-methylphenyl)-2-(2-methyl-6-(piperazin-1-yl)pyrimidin-4-ylamino)thiazole-5-carboxamide (Compound 109)

A mixture of compound 108 (2.60 g, 6.6 mmol), piperazine (5.60 g, 66.0 mmol), potassium carbonate (1.82 g, 13.2 mmol) and DMF (15 mL) was stirred at 135° C. for 12 hours. The solvent was evaporated under reduce pressure and the residue was washed with water, acetone and ethyl acetate in turn to obtain the title compound 109 as a pale yellow solid (1.8 g, 64%): LCMS: 444 [M+1]⁺.

### Step 1h. Ethyl 2-(4-(6-(5-(2-chloro-6-methylphenylcarbamoyl)thiazol-2-ylamino)-2-methylpyrimidin-4-yl)piperazin-1-yl)acetate (Compound 110-1)

A mixture of compound 109 (0.31 g, 0.70 mmol), ethyl 2-bromoacetate (117 mg, 0.70 mmol), triethylamine (0.28 g, 0.70 mmol) and DMF (5 mL) was stirred at 35° C. for 2 minutes. The solvent was evaporated under reduce pressure to give the title compound 110-1 as a white solid (333 mg, 90%) which was used directly to the next step without further purification: LCMS: 530 [M+1]⁺.

### Step 11. N-(2-Chloro-6-methylphenyl)-2-(6-(4-(2-(hydroxyamino)-2-oxoethyl)piperazin-1-yl)-2-methylpyrimidin-4-ylamino)thiazole-5-carboxamide (Compound 1)

To a stirred solution of hydroxylamine hydrochloride (4.67 g, 67.0 mmol) in methanol (24 mL) at 0° C. was added a solution of potassium hydroxide (5.61 g, 100.0 mmol) in methanol (14 mL). After addition, the mixture was stirred for 30 minutes at 0° C., and was allowed to stand at 0° C. The resulting precipitate was isolated, and the solution was prepared to give free hydroxylamine.

The above freshly prepared hydroxylamine solution (0.5 mL, 0.89 mmol) was placed in 5 mL flask. Compound 110-1 (333 mg, 0.63 mmol) was added to this solution under ultrasonic for 10 minutes. The reaction process was monitored by TLC. The mixture was neutralized with acetic acid and was then concentrated under reduce pressure. The residue was purified by preparative HPLC to give the title compound 1 as a white solid (50 mg, 16%): LCMS: 517 [M+1]⁺; ¹H NMR (DMSO-d₆) δ 2.20 (s, 3H), 2.38 (s, 3H), 2.58 (m, 4H), 2.90 (s, 2H), 3.51 (m, 4H), 6.02 (s, 1H), 7.26 (m, 2H), 7.37 (m, 1H), 8.20 (s, 1H), 8.80 (s, 1H), 9.86 (s, 1H), 10.47 (s, 1H), 11.46 (s, 1H).

### Example 2

### Preparation of N-(2-chloro-6-methylphenyl)-5-(6-(4-(3-(hydroxyamino)-3-oxopropyl)piperazin-1-yl)-2-methylpyrimidin-4-ylamino)-4H-pyrrole-3-carboxamide (Compound 2)

### Step 2a. Methyl 3-(4-(6-(5-(2-chloro-6-methylphenylcarbamoyl)thiazol-2-ylamino)-2-methylpyrimidin-4-yl)piperazin-1-yl)propanoate (Compound 110-2)

The title compound 110-2 was prepared as a pale yellow solid (0.31 g, 74%) from compound 109 (0.35 g, 0.79 mmol), methyl 3-bromopropanoate (0.13 g, 0.78 mmol), DIEA (0.21 g, 1.58 mmol) and DMF (5 mL) using a procedure similar to that described for compound 110-1 (Example 1): LCMS: 530 [M+1]⁺.

### Step 2b. N-(2-Chloro-6-methylphenyl)-2-(6-(4-(3-(hydroxyamino)-3-oxopropyl)piperazin-1-yl)-2-methylpyrimidin-4-ylamino)thiazole-5-carboxamide (Compound 2)

The title compound 2 was prepared as a white solid (60 mg, 19%) from compound 110-2 (0.31 g, 0.59 mmol) using a procedure similar to that described for compound 1 (Example 1): LCMS: 531 [M+1]⁺; ¹H NMR (DMSO-d₆) δ 2.16 (t, J=6.9 Hz, 2H), 2.24 (s, 3H), 2.41 (s, 3H), 2.54 (m, 4H), 2.57 (t, J=6.6 Hz, 2H), 3.50 (m, 4H), 6.05 (s, 1H), 7.25 (m, 2H), 7.37 (m, 1H), 8.23 (s, 1H), 8.88 (s, 1H), 9.90 (s, 1H), 10.42 (s, 1H), 11.51 (s, 1H).

### Example 3

### Preparation of N-(2-chloro-6-methylphenyl)-2-(6-(4-(4-(hydroxyamino)-4-oxobutyl)piperazin-1-yl)-2-methylpyrimidin-4-ylamino)thiazole-5-carboxamide (Compound 3)

### Step 3a. Ethyl 4-(4-(6-(5-(2-chloro-6-methylphenylcarbamoyl)thiazol-2-ylamino)-2-methylpyrimidin-4-yl)piperazin-1-yl)butanoate (Compound 110-3)

The title compound 110-3 was prepared as a pale yellow solid (0.22 g, 71%) from compound 109 (0.25 g, 0.56 mmol), ethyl 4-bromobutanoate (0.12 g, 0.56 mmol), DIEA (0.15 g, 0.56 mmol) and DMF (5 mL) using a procedure similar to that described for compound 110-1 (Example 1): LCMS: 558 [M+1]⁺.

### Step 3b. N-(2-chloro-6-methylphenyl)-2-(6-(4-(4-(hydroxyamino)-4-oxobutyl)piperazin-1-yl)-2-methylpyrimidin-4-ylamino)thiazole-5-carboxamide (Compound 3)

The title compound 3 was prepared as a white solid (30 mg, 14%) from compound 110-3 (0.22 g, 0.40 mmol) using a procedure similar to that described for compound 1 (Example 1): LCMS: 545 [M+1]⁺; ¹H NMR (DMSO-d₆) δ 1.69 (m, 2H), 2.01 (t, J=6.6 Hz, 2H), 2.25 (s, 3H), 2.30 (t, J=6.9, 2H), 2.41 (m, 4H), 2.55 (s, 3H), 3.52 (m, 4H), 6.06 (s, 1H), 7.25 (m, 2H), 7.36 (m, 1H), 8.23 (s, 1H), 8.70 (s, 1H), 9.90 (s, 1H), 10.37 (s, 1H), 11.50 (s, 1H).

### Example 4

### Preparation of N-(2-chloro-6-methylphenyl)-2-(6-(4-(5-(hydroxyamino)-5-oxopentyl)piperazin-1-yl)-2-methylpyrimidin-4-ylamino)thiazole-5-carboxamide (Compound 4)

### Step 4a. Methyl methyl 5-(4-(6-(5-(2-chloro-6-methylphenylcarbamoyl)thiazol-2-ylamino)-2-methylpyrimidin-4-yl)piperazin-1-yl)pentanoate (Compound 110-4)

The title compound 110-4 was prepared as a pale yellow solid (120 mg, 39%) from compound 109 (0.24 g, 0.54 mmol), methyl 5-bromopentanoate (0.12 g, 0.60 mmol), DIEA (1.54 g, 1.20 mmol) and DMF (3 mL) using a procedure similar to that described for compound 110-1 (Example 1): LCMS: 558 [M+1]⁺.

### Step 4b. N-(2-chloro-6-methylphenyl)-2-(6-(4-(5-(hydroxyamino)-5-oxopentyl)piperazin-1-yl)-2-methylpyrimidin-4-ylamino)thiazole-5-carboxamide (Compound 4)

The title compound 4 was prepared as a white solid (30 mg, 25%) from compound 110-4 (120 mg, 0.22 mmol) using a procedure similar to that described for compound 1 (Example 1): LCMS: 559 [M+1]⁺; ¹H NMR (DMSO-d₆) δ 1.44 (m, 4H), 1.95 (t, J=7.5 Hz, 2H), 2.22 (s, 3H), 2.26 (t, J=6.9 Hz, 2H), 2.37 (m, 7H), 3.47 (m, 4H), 6.07 (s, 1H), 7.25 (m, 2H), 7.37 (dd, J=2.1 Hz, J=7.2 Hz, 2H), 8.23 (s, 1H), 9.93 (s, 1H).

### Example 5

### Preparation of N-(2-chloro-6-methylphenyl)-2-(6-(4-(6-(hydroxyamino)-6-oxohexyl)piperazin-1-yl)-2-methylpyrimidin-4-ylamino)thiazole-5-carboxamide (Compound 5)

### Step 5a. Ethyl 6-(4-(6-(5-(2-chloro-6-methylphenylcarbamoyl)thiazol-2-ylamino)-2-methylpyrimidin-4-yl)piperazin-1-yl)hexanoate (Compound 110-5)

The title compound 110-5 was prepared as a brown solid (120 mg, 41%) from compound 109 (0.22 g, 0.495 mol), ethyl 6-bromohexanoate (0.12 g, 0.495 mmol), potassium carbonate (0.22 g, 1.60 mmol) and DMF (5 mL) using a procedure similar to that described for compound 110-1 (Example 1): LCMS: 586 [M+1]⁺.

### Step 5b. N-(2-Chloro-6-methylphenyl)-2-(6-(4-(6-(hydroxyamino)-6-oxohexyl)piperazin-1-yl)-2-methylpyrimidin-4-ylamino)thiazole-5-carboxamide (Compound 5)

The title compound 5 was prepared as a white solid (30 mg, 26%) from compound 110-5 (120 mg, 0.20 mmol) using a procedure similar to that described for compound 1 (Example 1): LC-MS: 573 [M+1]⁺; ¹H NMR (DMSO-d₆) δ 1.26 (m, 2H), 1.49 (m, 4H), 1.93 (t, J=7.2 Hz, 2H), 2.22 (s, 3H), 2.26 (t, J=7.2 Hz, 2H), 2.48 (m, 7H), 3.47 (m, 4H), 6.04 (s, 1H), 7.26 (m, 2H), 7.37 (m, 2H), 8.21 (s, 1H), 8.66 (s, 1H), 9.88 (s, 1H), 10.33 (s, 1H), 10.33 (s, 1H).

### Example 6

### Preparation of N-(2-chloro-6-methylphenyl)-2-(6-(2-(2-(hydroxyamino)-2-oxoethylamino)ethylamino)-2-methylpyrimidin-4-ylamino)thiazole-5-carboxamide (Compound 7)

### Step 6a. 2-(6-(2-Aminoethylamino)-2-methylpyrimidin-4-ylamino)-N-(2-chloro-6-methylphenyl)thiazole-5-carboxamide (Compound 201)

A solution of compound 108 (3.0 g, 7.6 mmol) in ethane-1,2-diamine (50 mL) was heated to 80° C. and stirred for 10 hours. The reaction was then concentrated under vacuum and the residue was partitioned between H₂O and EtOAc. The EtOAc phase was separated, washed with brine, dried (Na₂SO₄) and concentrated under vacuum to yield the title compound 201 as a brown solid (1.3 g, 40%). LC-MS: 418 [M+1]⁺, H-NMR (DMSO-d₆): δ 1.86 (s, 2H), 2.22 (s, 3H), 2.36 (s, 3H), 2.48 (t, J=6.0 Hz, 2H), 2.76 (t, J=6.0 Hz, 2H), 3.15 (s, 1H), 5.88 (s, 1H), 7.26 (m, 2H), 7.37 (dd, J=2.4, J=6.9 Hz, 1H), 8.19 (s, 1H), 9.83 (s, 1H).

### Step 6b. Ethyl 2-(2-(6-(5-(2-chloro-6-methylphenylcarbamoyl)thiazol-2-ylamino)-2-methylpyrimidin-4-yl amino)ethylamino)acetate (Compound 202-7)

A solution of compound 201 (0.50 g, 1.2 mmol) in DMF (15 mL) was added ethyl 2-bromoacetate (0.2 g, 1.2 mmol) and K₂CO₃ (41 mg, 0.3 mmol). The reaction was stirred at 30° C. for 2 hours. The mixture was concentrated under vacuum and the residue was purified by column chromatograph to obtain title compound 202-7 as a pale yellow solid (110 mg, 22%): LC-MS: 504 [M+1]⁺.

### Step 6c. N-(2-Chloro-6-methylphenyl)-2-(6-(2-(2-(hydroxyamino)-2-oxoethylamino)ethylamino)-2-methylpyrimidin-4-ylamino)thiazole-5-carboxamide (Compound 7)

The title compound 7 was prepared as a pale yellow solid (42 mg, 37%) from compound 202-7 (110 mg, 0.29 mmol) using a procedure similar to that described for compound 1 (Example 1): LC-MS: 491 [M+1]⁺, H-NMR (DMSO-d₆): δ 2.21 (s, 3H), 2.34 (s, 3H), 2.60 (t, J=6 Hz, 2H), 3.03 (s, 2H), 3.11 (t, J=5.7 Hz, 2H), 5.86 (s, 1H), 7.22 (m, 2H), 7.36 (dd, J=2.1, J=7.2 Hz, 1H), 8.18 (s, 1H), 9.83 (s, 1H).

### Example 7

### Preparation of N-(2-chloro-6-methylphenyl)-2-(6-(4-(7-(hydroxyamino)-7-oxoheptyl)piperazin-1-yl)-2-methylpyrimidin-4-ylamino)thiazole-5-carboxamide (Compound 6)

### Step 7a. ethyl 7-(4-(6-(5-(2-chloro-6-methylphenylcarbamoyl)thiazol-2-ylamino)-2-methylpyrimidin-4-yl)piperazin-1-yl)heptanoate (Compound 110-6)

The title compound 110-6 was prepared as a brown solid (176 mg, 59%) from compound 109 (0.22 g, 0.50 mmol), ethyl 7-bromoheptanoate (0.12 g, 0.506 mmol), diisopropylethylamine (0.13 g, 1.00 mmol) and DMF (5 mL) using a procedure similar to that described for compound 110-1 (Example 1): LCMS: 600 [M+1].

### Step 7b. N-(2-chloro-6-methylphenyl)-2-(6-(4-(6-(hydroxyamino)-7-oxoheptyl)piperazin-1-yl)-2-methylpyrimidin-4-ylamino)thiazole-5-carboxamide (Compound 6)

The title compound 6 was prepared as a white solid (32 mg, 82%) from compound 110-6 (40 mg, 0.067 mmol) using a procedure similar to that described for compound 1 (Example 1): LC-MS: 587 [M+1]⁺; ¹H NMR (DMSO-d₆) δ 1.24 (m, 4H), 1.44 (m, 4H), 1.92 (t, J=7.2 Hz, 2H), 2.22 (s, 3H), 2.26 (t, J=6.3 Hz, 2H), 2.38 (ds, 7H), 3.48 (m, 4H), 6.03 (s, 1H), 7.26 (m, 2H), 7.37 (m, 1H), 8.19 (s, 1H), 8.63 (ds, 1H), 9.83 (s, 1H), 10.28 (s, 1H), 11.43 (s, 1H).

### Example 8

### Preparation of N-(2-chloro-6-methylphenyl)-2-(6-(2-(3-(hydroxyamino)-3-oxopropylamino)ethylamino)-2-methylpyrimidin-4-ylamino)thiazole-5-carboxamide (Compound 8)

### Step 8a. Methyl 3-(2-(6-(5-(2-chloro-6-methylphenylcarbamoyl)thiazol-2-ylamino)-2-methylpyrimidin-4-ylamino)ethylamino)propanoate (Compound 202-8)

The title compound 202-8 was prepared as a white solid (400 mg, 31%) from compound 201 (1.08 g, 2.6 mol), methyl 4-bromobutanoate (0.44 g, 2.6 mmol) and K₂CO₃ (0.44 mg, 5.2 mmol) using a procedure similar to that described for compound 202-7 (Example 6): LCMS 504 [M+1]¹H-NMR ((DMSO-d₆): δ 2.22 (s, 3H), 2.38 (s, 3H), 2.64 (t, J=6.9 Hz, 2H), 2.93 (t, J=6.0 Hz, 2H), 3.03 (t, J=6.6 Hz, 2H), 3.61 (s, 3H), 7.26 (m, 3H), 7.39 (m, 1H), 8.22 (s, 1H), 9.88 (s, 1H).

### Step 8b. N-(2-chloro-6-methylphenyl)-2-(6-(2-(3-(hydroxyamino)-3-oxopropylamino)ethylamino)-2-methylpyrimidin-4-ylamino)thiazole-5-carboxamide (Compound 8)

The title compound 8 was prepared as a off white solid (30 mg, 60%) from compound 202-8 (51 mg, 0.10 mmol) using a procedure similar to that described for compound 1 (Example 1): LCMS 505 [M+1]⁺; ¹H NMR (DMSO-d₆), δ 2.13 (t, J=6.9 Hz 2H), 2.22 (s, 3H), 2.36 (s, 3H), 2.70 (t, J=6.6 Hz, 2H), 2.77 (t, J=6.9 Hz, 2H), 5.87 (s, 1H), 7.21 (m, 3H), 7.39 (m, 1H), 8.19 (s, 1H), 9.84 (s, 1H).

### Example 9

### Preparation of N-(2-chloro-6-methylphenyl)-2-(6-(2-(6-(hydroxyamino)-6-oxohexylamino)ethylamino)-2-methylpyrimidin-4-ylamino)thiazole-5-carboxamide (Compound 11)

### Step 9a. Ethyl 6-(2-(6-(5-(2-chloro-6-methylphenylcarbamoyl)thiazol-2-ylamino)-2-methylpyrimidin-4-ylamino)ethylamino)hexanoate (Compound 202-11)

The title compound 202-11 was prepared as a pale yellow solid (100 mg, 17%) from compound 201 (0.50 g, 1.2 mol), ethyl ethyl 6-bromohexanoate (0.27 g, 1.2 mmol) and K₂CO₃ (41 mg, 0.3 mmol) using a procedure similar to that described for compound 202-7 (Example 6): H-NMR (CDCl₃): δ 1.24 (m, 5H), 1.41 (m, 2H), 1.57 (m, 2H), 2.23 (t, J=7.2 Hz, 2H), 2.34 (s, 3H), 2.50 (s, 3H), 2.57 (t, J=5.7 Hz, 2H), 2.84 (t, J=5.7 Hz, 2H), 3.37 (m, 2H), 4.11 (q, J=7.2 Hz, 2H), 5.46 (ds, 1H), 5.70 (s, 1H), 7.16 (m, 1H), 7.29 (m, 3H), 8.15 (s, 1H).

### Step 9b. N-(2-chloro-6-methylphenyl)-2-(6-(2-(6-(hydroxyamino)-6-oxohexylamino)ethylamino)-2-methylpyrimidin-4-ylamino)thiazole-5-carboxamide (Compound 11)

The title compound 11 was prepared as a off white solid (34 mg, 33%) from compound 202-11 (100 mg, 0.18 mmol) using a procedure similar to that described for compound 1 (Example 1): LCMS 547 [M+1]⁺; ¹H NMR (DMSO-d₆), δ 1.25 (m, 2H), 1.47 (m, 4H), 1.95 (t, J=7.2 Hz 2H), 2.21 (s, 3H), 2.37 (s, 3H), 2.75 (t, J=6.9 Hz, 2H), 2.91 (t, J=6.6 Hz, 2H), 3.42 (ds, 1H), 5.90 (s, 1H), 7.22 (m, 4H), 8.19 (s, 1H), 9.8 (s, 1H), 10.4 (ds, 1H).

### Example 10

### Preparation of N-(2-chloro-6-methylphenyl)-2-(6-(6-(hydroxyamino)-6-oxohexylamino)-2-methylpyrimidin-4-ylamino)thiazole-5-carboxamide (Compound 23)

### Step 10a. Methyl6-(6-(5-(2-chloro-6-methylphenylcarbamoyl)thiazol-2-ylamino)-2-methylpyrimidin-4-ylamino)hexanoate (Compound 301-23)

A solution of compound 108 (240 mg, 0.61 mmol), DMAC (15 mL), KOH (170 mg, 3.05 mmol) and methyl 6-aminohexanoate (554 mg, 3.05 mmol) was stirred for 12 h at 120° C. The reaction mixture was diluted with water, filtered and dried to give the crude compound 301-23 as a pale yellow powder (88 mg, 30%) which was used directly to next step without further purification. LCMS: 503 [M+1]⁺.

### Step 10b. N-(2-chloro-6-methylphenyl)-2-(6-(6-(hydroxyamino)-6-oxohexylamino)-2-methylpyrimidin-4-ylamino)thiazole-5-carboxamide (Compound 23)

A mixture of compound 301-23 (88 mg, 0.18 mmol) and freshly prepared NH₂OH methanol solution (1.77 M, 2.10 mL) was stirred for 30 min at room temperature. The mixture was adjusted to pH=7.0 with AcOH and the solvent was removed. The resulting residue was purified by column chromatography to give the title compound 23 as a white powder (25 mg, 29%): LCMS: 504 [M+1]⁺; ¹H NMR (DMSO-d₆) δ 11.30 (s, 1H), 10.29 (s, 1H), 9.80 (s, 1H), 8.60 (s, 1H), 8.17 (s, 1H), 7.38 (dd, 1H, J=2.1, J=7.2 Hz), 7.25 (m, 2H), 7.12 (m, 1H), 5.83 (s, 1H), 3.13 (brs, 2H), 2.34 (s, 3H), 2.22 (s, 3H), 1.93 (m, 2H), 1.50 (m, 1H), 1.26 (m, 2H).

### Example 11

### Preparation of N-(2-chloro-6-methylphenyl)-2-(6-(7-(hydroxyamino)-7-oxoheptylamino)-2-methylpyrimidin-4-ylamino)thiazole-5-carboxamide (Compound 24)

### Step 11a. Methyl 7-(6-(5-(2-chloro-6-methylphenylcarbamoyl)thiazol-2-ylamino)-2-methylpyrimidin-4-ylamino)heptanoate (Compound 301-24)

The title compound 301-24 was prepared as a crude pale yellow solid (120 mg, 38%) from compound 108 (240 mg, 0.61 mmol), DMAC (15 mL), KOH (170 mg, 3.05 mmol) and methyl 7-aminoheptanoate (596 mg, 3.05 mmol) using a procedure similar to that described for compound 301-23 (Example 10): LCMS: 517 [M+1]⁺.

### Step 11b. N-(2-chloro-6-methylphenyl)-2-(6-(7-(hydroxyamino)-7-oxoheptylamino)-2-methylpyrimidin-4-ylamino)thiazole-5-carboxamide (Compound 24)

The title compound 24 was prepared as a white solid (35 mg, 30%) from compound 301-24 (120 mg, 0.23 mmol) and freshly prepared hydroxylamine methanol solution (1.77 M, 3.28 mL) using a procedure similar to that described for compound 23 (Example 10): m.p. 150.7° C. (decomp.), LCMS: 518 [M+1]; ¹H NMR (DMSO-d₆) δ 11.37 (s, 1H), 10.33 (s, 1H), 9.85 (s, 1H), 8.66 (s, 1H), 8.18 (s, 1H), 7.39 (dd, 1H, J=2.1, J=7.2 Hz), 7.26 (m, 2H), 7.19 (m, 1H), 5.82 (s, 1H), 3.14 (brs, 2H), 2.34 (s, 3H), 2.22 (s, 3H), 1.92 (m, 2H), 1.47 (m, 4H), 1.27 (m, 4H).

### Biological Assays:

As stated hereinbefore the derivatives defined in the present invention possess anti-proliferation activity. These properties may be assessed, for example, using one of the procedures set out below:

(a) An In Vitro Assay which Determines the Ability of a Test Compound to Inhibit a Tyrosine Kinase.

The ability of compounds to inhibit tyrosine kinase (Abl1, Src, c-Kit, and PDGFR-beta) activity is assayed using HTScan™ Receptor Kinase Assay Kits (Cell Signaling Technologies, Danvers, Mass.). Abl1 tyrosine kinase is obtained in partially purified form from GST-kinase fusion protein which is produced using a baculovirus expression system from a construct expressing human Abl1 (Pro118-Ser553) (GenBank Accession No. NM_{—}005157) with an amino-terminal GST tag. Src tyrosine kinase is obtained in partially purified form from GST-kinase fusion protein which is produced using a baculovirus expression system from a construct expressing full length human Src (Met1-Leu536) (GenBank Accession No. NM_{—}005417) with an amino-terminal GST tag. c-Kit tyrosine kinase is obtained in partially purified form from GST-kinase fusion protein which is produced using a baculovirus expression system from a construct expressing human c-Kit (Thr544-Val976) with an amino-terminal GST tag. PDGFR-beta tyrosine kinase was produced using a baculovirus expression system from a construct containing a human PDGFR-beta c-DNA (GenBank Accession No. NM_{—}002609) fragment (Arg561-Leu1106) amino-terminally fused to a GST-HIS6-Thrombin cleavage site. The proteins are purified by one-step affinity chromatography using glutathione-agarose. An anti-phosphotyrosine monoclonal antibody, P-Tyr-100, is used to detect phosphorylation of biotinylated substrate peptides (Abl1 and Src, Biotin-Signal Transduction Protein (Tyr160); c-Kit, Biotinylated-KDR (Tyr996); PDGFR-β, Biotinylated-FLT3 (Tyr589)). Enzymatic activity is tested in 60 mM HEPES, 5 mM MgCl2 5 mM MnCl2 200 μM ATP, 1.25 mM DTT, 3 μM Na3VO4, 1.5 mM peptide, and 50 ng EGF Recpetor Kinase. Bound antibody is detected using the DELFIA system (PerkinElmer, Wellesley, Mass.) consisting of DELFIA® Europium-labeled Anti-mouse IgG (PerkinElmer, #AD0124), DELFIA® Enhancement Solution (PerkinElmer, #1244-105), and a DELFIA® Streptavidin coated, 96-well Plate (PerkinElmer, AAAND-0005). Fluorescence is measured on a WALLAC Victor 2 plate reader and reported as relative fluorescence units (RFU). Data are plotted using GraphPad Prism (v4.0a) and IC50's are calculated using a sigmoidal dose response curve fitting algorithm.

Test compounds are dissolved in dimethylsulphoxide (DMSO) to give a 20 mM working stock concentration. Each assay is setup as follows: 100 μl of 10 mM ATP is added to 1.25 ml 6 mM substrate peptide. The mixture is diluted with dH₂0 to 2.5 ml to make 2×ATP/substrate cocktail ([ATP]=400 mM, [substrate]=3 mM). The enzyme is immediately transferred from −80° C. to ice. The enzyme is allowed to thaw on ice. The mixture is microcentrifuged briefly at 4° C. to bring liquid to the bottom of the vial and returned immediately to ice. 10 μl of DTT (1.25 mM) is added to 2.5 ml of 4×HTScan™ Tyrosine Kinase Buffer (240 mM HEPES pH 7.5, 20 mM MgCl₂, 20 mM MnCl, 12 mM NaVO₃) to make DTT/Kinase buffer. 1.25 ml of DTT/Kinase buffer is transferred to enzyme tube to make a 4× reaction cocktail ([enzyme]=4 ng/μL in 4× reaction cocktail). 12.5 μl of the 4× reaction cocktail is incubated with 12.5 μl/well of prediluted compound of interest (usually around 10 μM) for 5 minutes at room temperature. 25 μl of 2×ATP/substrate cocktail is added to 25 μl/well preincubated reaction cocktail/compound. The reaction plate is incubated at room temperature for 30 minutes. 50 μl/well Stop Buffer (50 mM EDTA, pH 8) is added to stop the reaction. 25 μl of each reaction and 75 μl dH₂O/well is transferred to a 96-well streptavidin-coated plate and incubated at room temperature for 60 minutes. The plate is washed three times with 200 μl/well PBS/T (PBS, 0.05% Tween-20). The primary antibody, Phospho-Tyrosine mAb (P-Tyr-100), is diluted 1:1000 in PBS/T with 1% bovine serum albumin (BSA). 100 μl/well primary antibody is added and the mixture is incubated at room temperature for 60 minutes. The plates are again washed three times with 200 μl/well PBS/T. Europium labeled anti-mouse IgG is diluted 1:500 in PBS/T with 1% BSA. 100 μl/well diluted antibody is added and the mixture is incubated at room temperature for 30 minutes. The plate is washed five times with 200 μl/well PBS/T. 100 μl/well DELFIA® Enhancement Solution is added and the mixture is incubated at room temperature for 5 minutes. 615 nm fluorescence emission is detected using an appropriate Time-Resolved Plate Reader.

(b) An In Vitro Assay which Determines the Ability of a Test Compound to Inhibit HDAC Enzymatic Activity.

HDAC inhibitors is screened using an HDAC fluorimetric assay kit (AK-500, Biomol, Plymouth Meeting, Pa.). Test compounds are dissolved in dimethylsulphoxide (DMSO) to give a 20 mM working stock concentration. Fluorescence is measured on a WALLAC Victor 2 plate reader and reported as relative fluorescence units (RFU). Data are plotted using GraphPad Prism (v4.0a) and IC50's calculated using a sigmoidal dose response curve fitting algorithm.

Each assay is setup as follows: Defrost all kit components and kept on ice until use. Dilute HeLa nuclear extract 1:29 in Assay Buffer (50 mM Tris/Cl, pH 8.0, 137 mM NaCl, 2.7 mM KCl, 1 mM MgCl2). Prepare dilutions of Trichostatin A (TSA, positive control) and tested compounds in assay buffer (5× of final concentration). Dilute Fluor de Lys™ Substrate in assay buffer to 100 uM (50 fold=2× final). Dilute Fluor de Lys™ developer concentrate 20-fold (e.g. 50 μl plus 950 μl Assay Buffer) in cold assay buffer. Second, dilute the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Add Assay buffer, dilute trichostatin A or test inhibitor to appropriate wells of the microtiter plate. Add diluted HeLa extract or other HDAC sample to all wells except for negative controls. Allow diluted Fluor de Lys™ Substrate and the samples in the microtiter plate to equilibrate to assay temperature (e.g. 25 or 37° C. Initiate HDAC reactions by adding diluted substrate (25 μl) to each well and mixing thoroughly. Allow HDAC reactions to proceed for 1 hour and then stopped them by addition of Fluor de Lys™ Developer (50 μl). Incubate plate at room temperature (25° C.) for 10-15 min. Read samples in a microtiter-plate reading fluorimeter capable of excitation at a wavelength in the range 350-380 nm and detection of emitted light in the range 440-460 nm.

The following TABLE 5-B lists compounds representative of the invention and their activity in HDAC, SRC, c-Kit, PDGF and ABL assays. In these assays, the following grading was used: I≧10 μM, 10 μM>II>1 μM, 1 μM>III>0.1 μM, and IV≦0.1 μM for IC₅₀.

TABLE 5-B

| Compound | | | | | | | |
|---|---|---|---|---|---|---|---|
| No. | HDAC | ABL | SRC | c-Kit | PDGFb | Lyn | Lck |
| 1 | II | IV | IV | | IV | | |
| 2 | II | IV | IV | | IV | | |
| 3 | II | IV | IV | | | | |
| 4 | III | IV | IV | | IV | | |
| 5 | IV | IV | IV | IV | IV | IV | |
| 6 | III | IV | IV | IV | IV | IV | |
| 7 | I | IV | IV | | | | |
| 11 | IV | IV | IV | IV | IV | IV | IV |
| 23 | IV | IV | IV | IV | IV | IV | IV |
| 24 | IV | IV | IV | IV | IV | IV | IV |
| 30 | I | III | III | | | | |

TABLE 6-A

| | |
|---|---|
| | (X) |
| 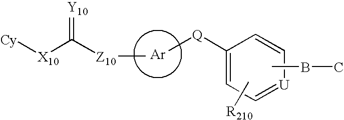 | |

SECTION 6:

| | |
|---|---|
| | (XI) |
| 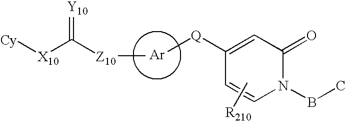 | |

| Compound # | Structure |
|---|---|
| 1 | 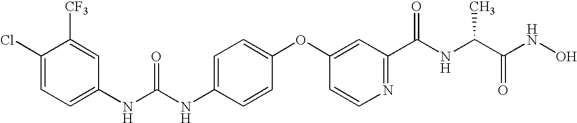 |
| 2 | 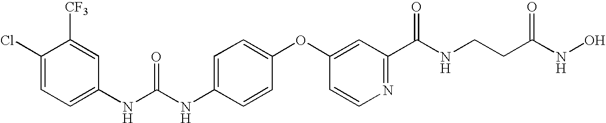 |
| 3 | 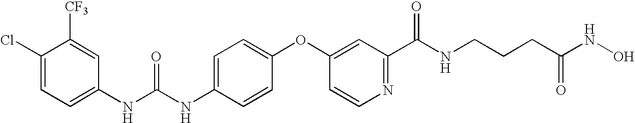 |
| 4 | 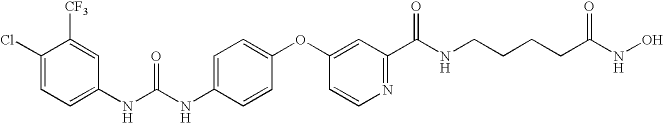 |
| 5 | 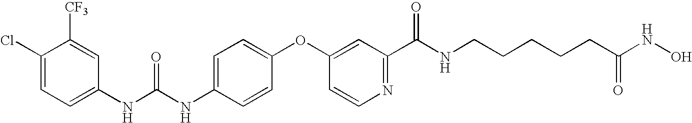 |
| 6 | 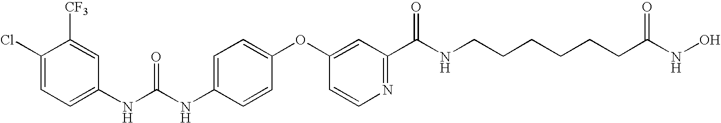 |
| 7 | 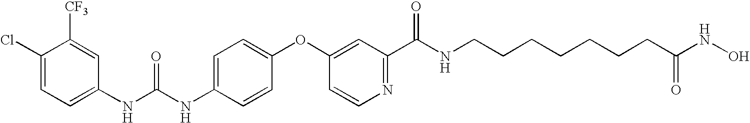 |
| 8 | 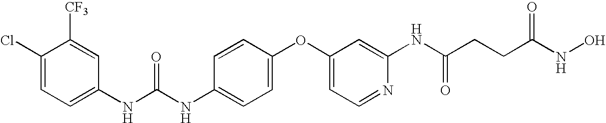 |
| 9 | 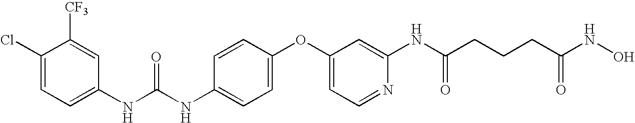 |
| 10 | 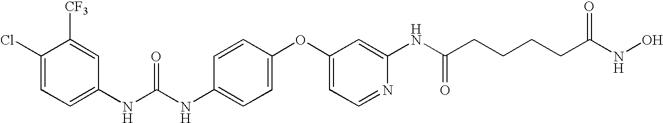 |
| 11 | 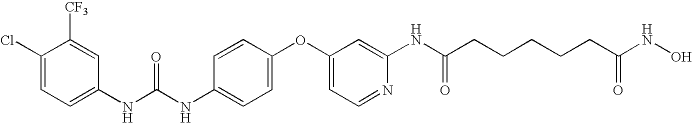 |
| 12 | 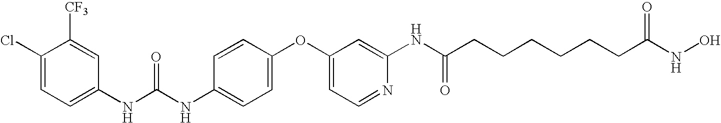 |
| 13 | 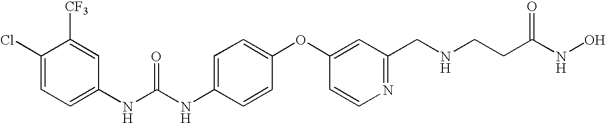 |
| 14 | 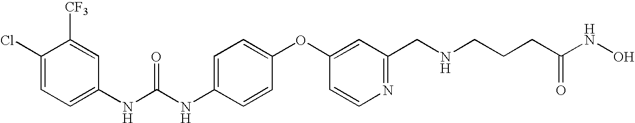 |
| 15 | 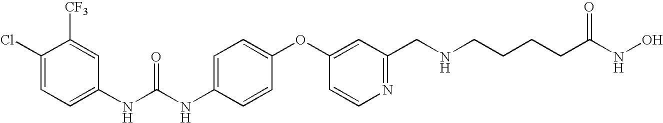 |
| 16 | 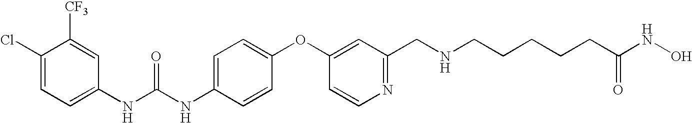 |
| 17 | 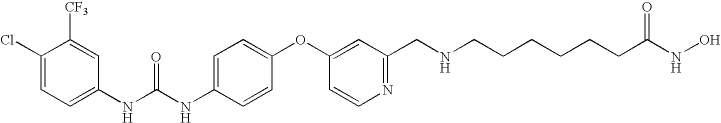 |
| 18 | 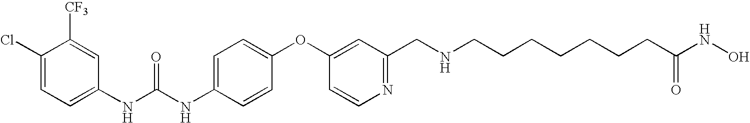 |
| 19 | 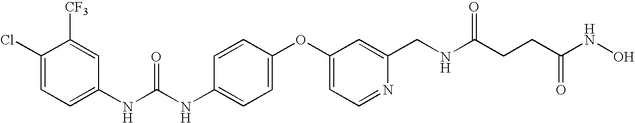 |
| 20 | 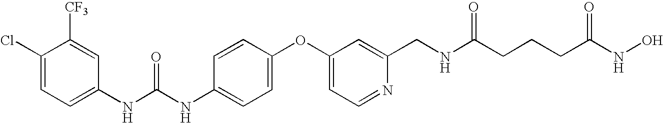 |
| 21 | 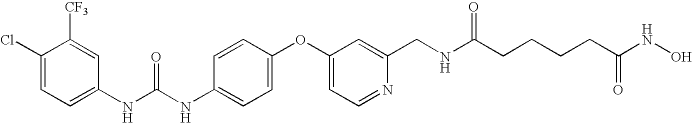 |
| 22 | 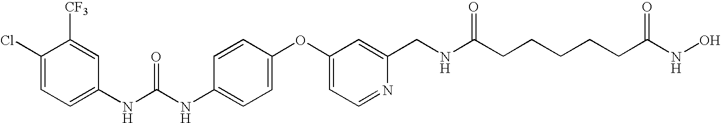 |
| 23 | 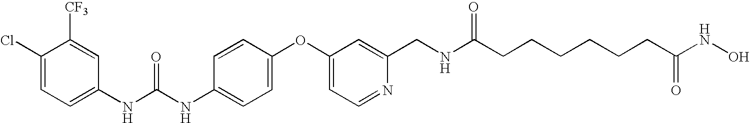 |
| 24 | 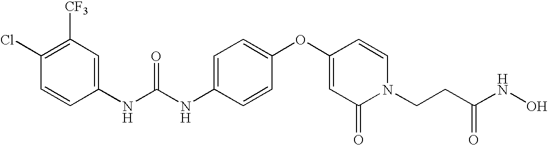 |
| 25 | 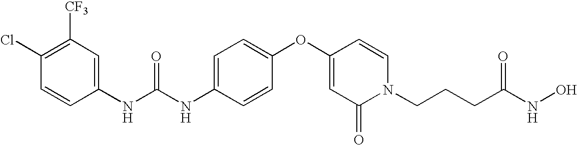 |
| 26 | 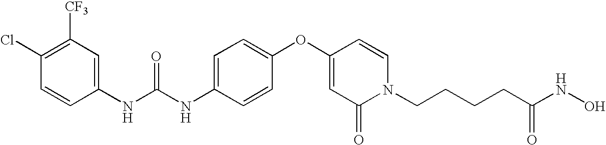 |
| 27 | 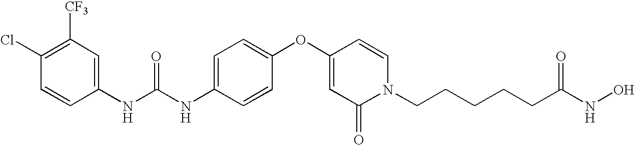 |
| 28 | 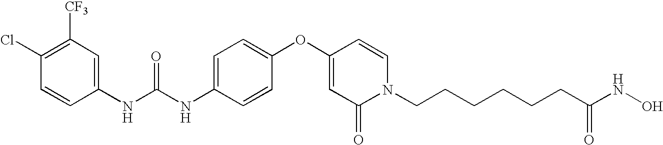 |
| 29 | 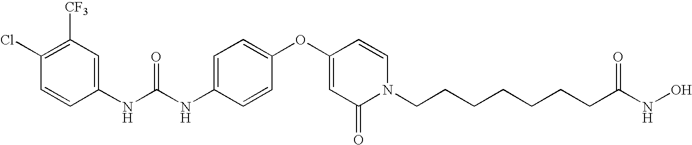 |
| 30 | 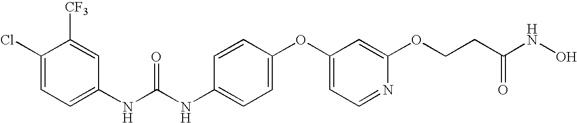 |
| 31 | 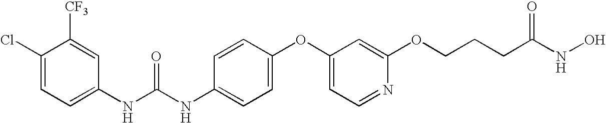 |
| 32 | 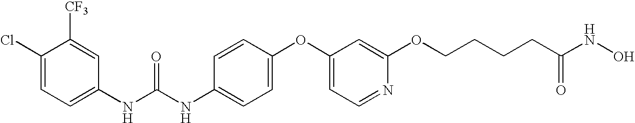 |
| 33 | 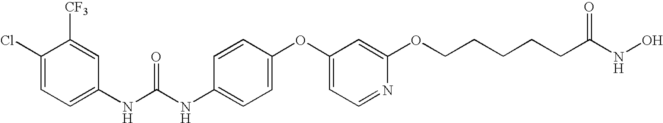 |
| 34 | 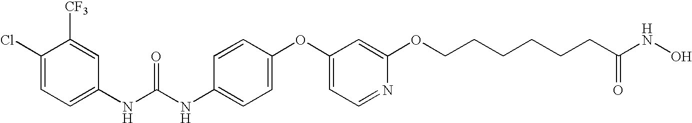 |
| 35 | 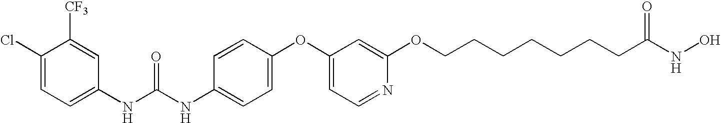 |
| 36 | 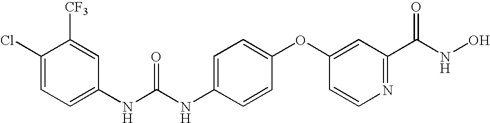 |

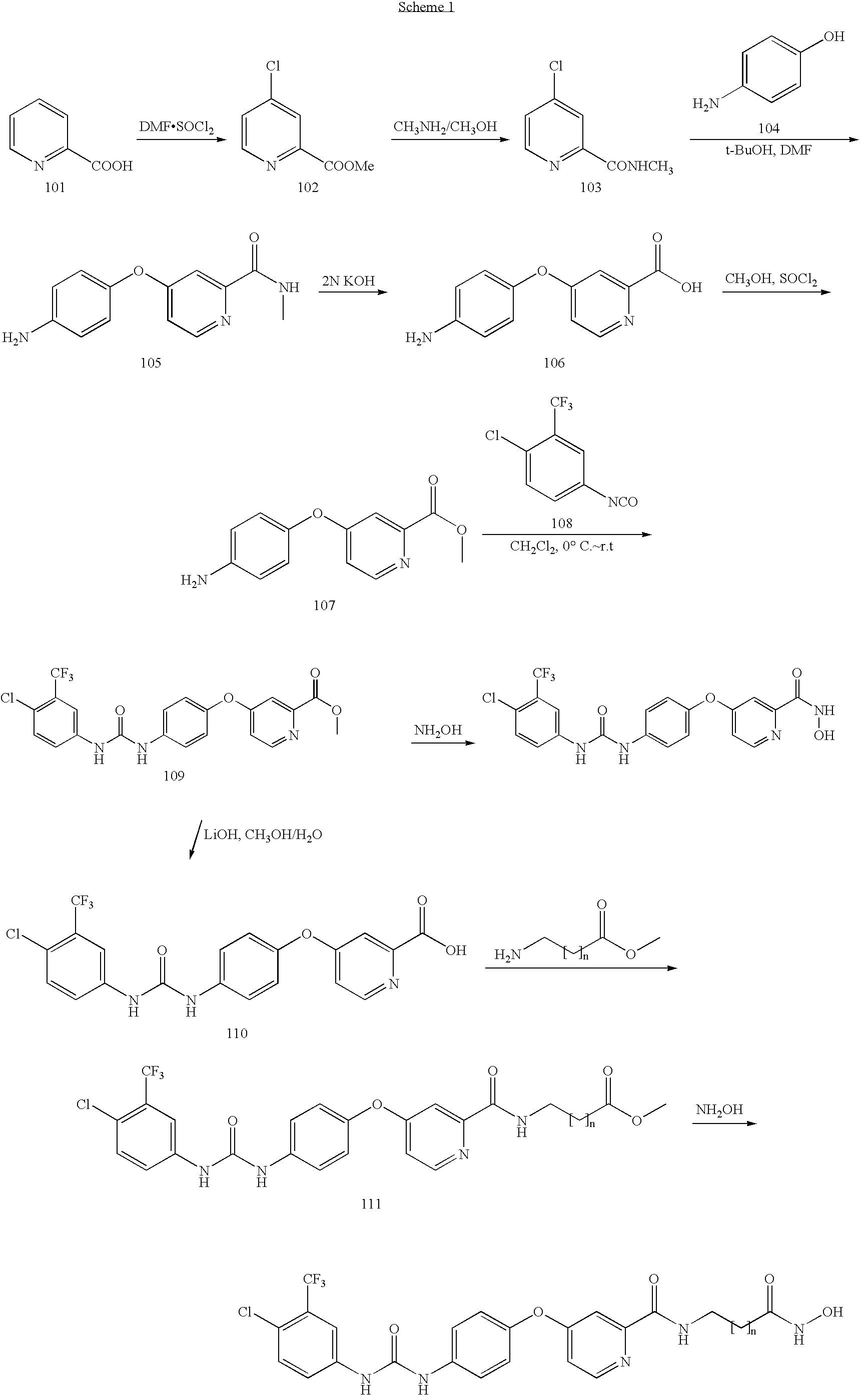

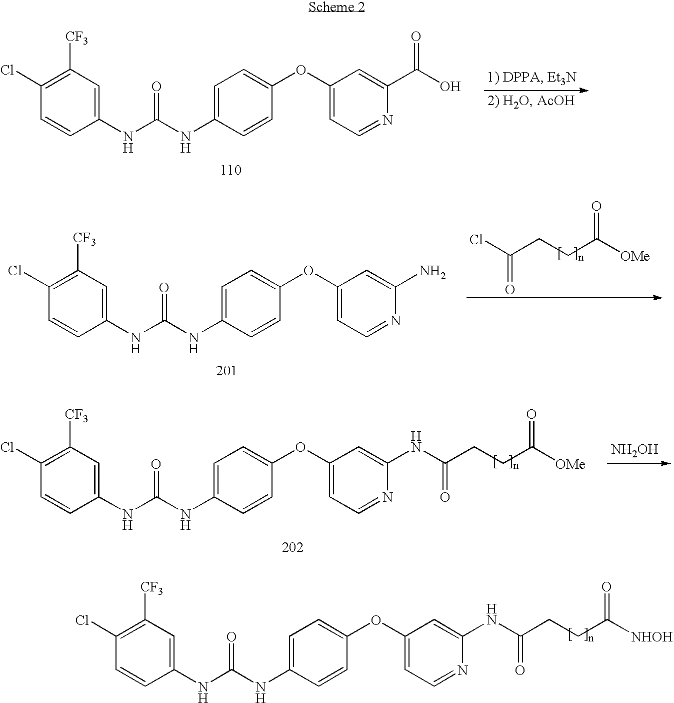

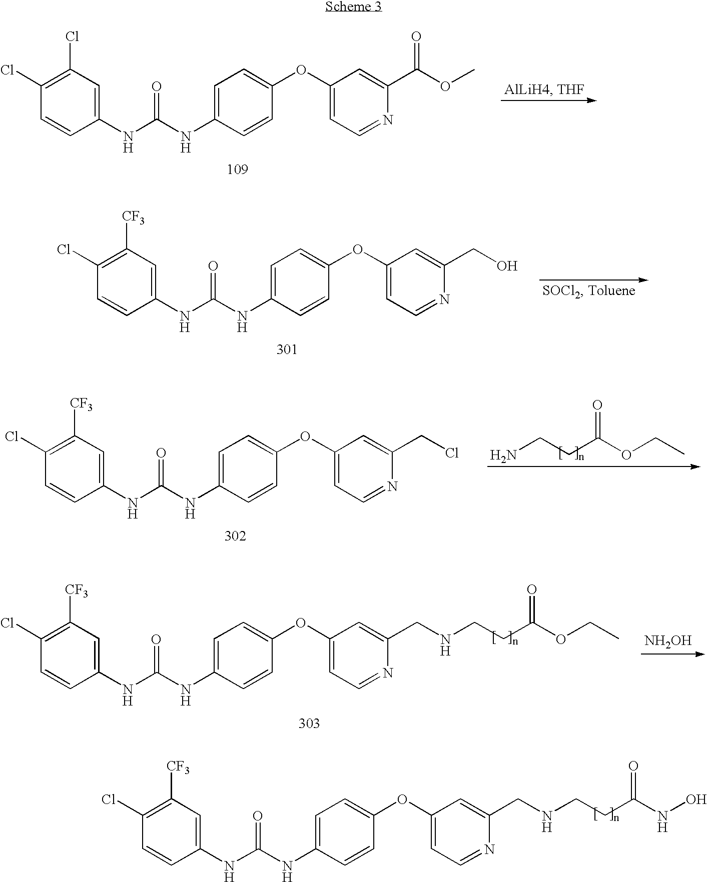

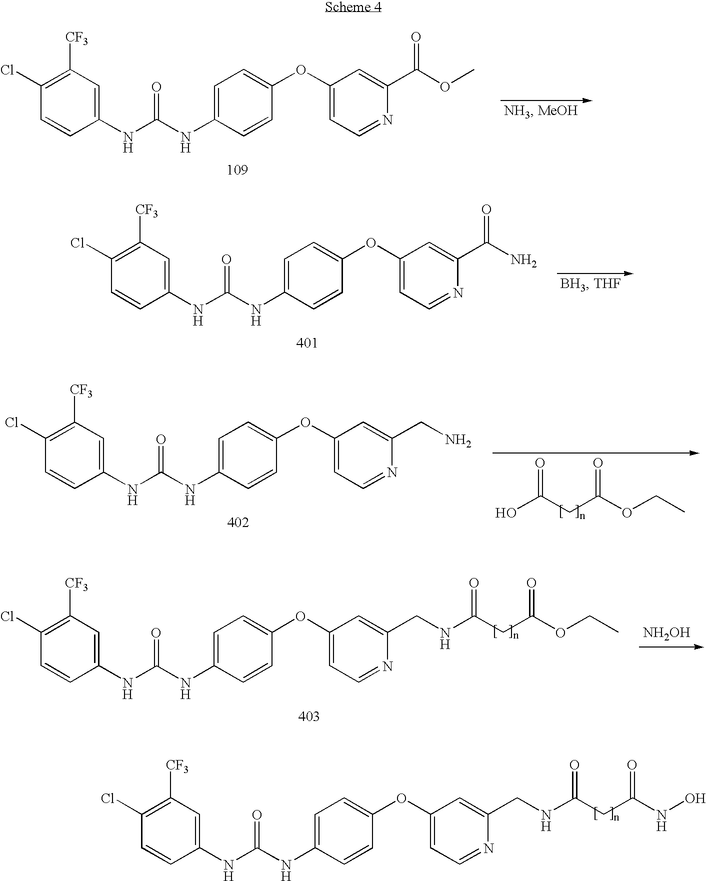

### Example 1

### Preparation of (R)-4-(4-(3-(4-chloro-3-(trifluoromethyl)phenyl)ureido)phenoxy)-N-(1-(hydroxyamino)-1-oxopropan-2-yl)picolinamide (Compound 1)

### Step 1a. Methyl 4-chloropicolinate (Compound 102)

Anhydrous DMF (10 mL) was slowly added to SOCl₂ (300 mL) at 40-48° C. The solution was stirred at room temperature for 10 minutes, and then compound 101 (100.0 g, 813.0 mmol) was added over 30 minutes. The resulting solution was heated at 72° C. (Vigorous SO₂ evolution) for 16 h to generate a yellow solid. The resting mixture was cooled to room temperature, diluted with toluene (500 mL) and concentrated to 200 mL. The toluene addition/concentration process was repeated twice. The resulting solution and solid was added into 200 mL methanol at ice bath to keep the internal temperature below 55° C. The content were stirred at r.t. for 45 min, cooled to 5° C. and treated with Et₂O (200 mL) dropwise. The resulting solid were filtered, washed with Et₂O (200 mL) and dried under 35° C. to provide a white yellow solid. After the solid were solvated to hot water (500 mL, about 45° C.), NaHCO₃ was added to adjust pH to 8-9. The mixture was extracted with ethyl acetate and the organic phase was concentrated to give desired compound 102 as a off-white solid (118.2 g, 85%). LCMS: 172 [M+1]⁺.

### Step 1b. 4-Chloro-N-methylpicolinamide (Compound 103)

To a methanol solution (4 mL) of compound 102 (10.0 g, 58.6 mmol) was added CH₃NH₂ (7.3 g, 234.4 mmol) in methanol at the temperature below 5° C. The mixture was stirred at 0-5° C. for 2 h. The solvent was evaporated at 40-50° C. to give the title compound 103 as a black yellow solid (9.8 g, 98%). LCMS: 171 [M+1]⁺; ¹H NMR (DMSO-d₆): δ 2.80 (d, 3H), 7.68 (dd, J₁=5.4 Hz, J₂=2.4 Hz, 1H), 7.97 (d, J=2.4 Hz, 1H), 8.56 (d, 1H), 8.82 (s, 1H).

### Step 1c. 4-(4-aminophenoxy)-N-methylpicolinamide (Compound 105)

A solution of 4-aminophenol (104) (9.6 g, 88.0 mmol) in anhydrous DMF (150 mL) was treated with t-BuOK (10.29 g, 91.7 mmol). The resulting reddish-brown mixture was stirred at room temperature for 2 h and was then added K₂CO₃ (6.5 g, 47 mmol) and compound 103 (15.0 g, 87.9 mmol). The reaction was stirred at 72° C. overnight and the solvent was evaporated at 50-60° C. to leave a reaction mixture. The mixture was cooled and saturated NaCl solution was added. The mixture was extracted with ethyl acetate. The organic layer was separated and washed with saturated NaCl solution, dried with Na₂SO₄ and concentrated under reduced pressure to afford compound 105 as a light-brown solid (17.9 g, 84%) with was used directly in the next step without further purification. LCMS: 244 [M+1]⁺.

### Step 1d. 4-(4-Aminophenoxy)picolinic acid (compound 106)

Compound 105 (32.4 g, 130.0 mol) was added into a solution of 2 N KOH (200 mL). The mixture was stirred at 100° C. for 2 hours. After the mixture was wash with EtOAc, the aqueous layer was adjusted to pH5. The water in the aqueous phase was removed by reduced pressure to leave a residue. A little water was added into this residue and filtrated. The collected solid was washed with a little water and dried to give 106 (23.9 g, 80%): LCMS: 231 [M+1]⁺; ¹H NMR (DMSO-d⁶): δ 6.66 (dd, J=8.7 Hz, 2H), 6.88 (dd, J=8.7 Hz, 2H), 7.12 (dd, J₁=5.4 Hz, J₂=2.7 Hz, 1H), 7.37 (d, J=2.4 Hz, 1H), 8.52 (d, J=5.4 Hz, 1H).

### Step 1e. Methyl 4-(4-aminophenoxy)picolinate (Compound 107)

SOCl₂ (6 mL) was added dropwise into a solution of compound 106 (4.0 g, 8.8 mmol) in methanol (50 mL) at below 0° C. The mixture was allowed to stir at 70° C. overnight. The solvent was evaporated and EtOAc and water were added. The PH value was adjusted to 8-9 with NaCO₃ and NaOH. The mixture was extracted with EtOAc three times. The organic phase was collected and concentrated to give crude product which was purified by column chromatography to yield the title compound 107 (2.1 g, 68%): LCMS: 245 [M+1]⁺.

### Step 1f. Methyl 4-(4-(3-(4-chloro-3-(trifluoromethyl)phenyl)ureido)phenoxy)picolinate (Compound 109)

A solution of 4-chloro-3-(trifluoromethyl)phenyl isocyanate (108) (4.97 g, 20.0 mmol) in CH₂Cl₂ (12 mL) was added dropwise to a suspension of compound 107 (4.50 g, 20.0 mmol) in CH₂Cl₂ (12 mL) at 0° C. The resulting mixture was stirred at room temperature for 22 h. The resulting yellow solid was collected by filtration and washed with CH₂Cl₂ (2×10 mL) to afford compound 109 as an off-white solid (7.90 g, 85%): LCMS: 466 [M+1].

### Step 1g. 4-(4-(3-(4-Chloro-3-(trifluoromethyl)phenyl)ureido)phenoxy)picolinic acid (Compound 110)

LiOH.H₂O (1.08 g, 25.60 mmol) was added into a solution of compound 109 (3.0 g, 6.4 mmol) in 8 mL methanol. Water (4 mL) was added into above mixture immediately. The reaction mixture was stirred at room temperature for 1 h. The PH value of above mixture was adjusted to 5 and methanol was evaporated. The resulting solid was filtrated to provide compound 110 as a gray solid (2.66 g, 92%): LCMS: 452 [M+1]⁻.

### Step 1h. (R)-Methyl 2-(4-(4-(3-(4-chloro-3-(trifluoromethyl)phenyl)ureido)phenoxy)picolinamido)propanoate (Compound 111-1)

Et₃N (336.0 mg, 3.3 mmol) was added into a solution of methyl 3-aminopropanoate hydrochloride (130.0 mg, 0.93 mmol) in 6 mL DMF. To the above mixture was then added compound 110 (300.0 mg, 0.67 mmol), HOBt (135.0 mg, 0.998 mmol) and EDCI (191.0 mg, 0.998 mmol). The mixture was stirred at room temperature for 18 h. Solvent DMF was evaporated at 50° C. and 100 mL ethyl acetate and 10 mL water were added. The organic phase was washed with water, dried over Na₂SO₄ and evaporated. The title compound 111-1 was purified by column chromatography (242.0 mg, 68%): LCMS: 537 [M+1]⁺.

### Step 1i. (R)-4-(4-(3-(4-Chloro-3-(trifluoromethyl)phenyl)ureido)phenoxy)-N-(1-(hydroxyamino)-1-oxopropan-2-yl)picolinamide (Compound 1)

To a stirred solution of hydroxylamine hydrochloride (4.67 g, 67.0 mmol) in methanol (24 mL) at 0° C. was added a solution of potassium hydroxide (5.61 g, 100.0 mmol) in methanol (14 mL). After addition, the mixture was stirred for 30 minutes at 0° C., and was allowed to stand at low temperature. The resulting precipitate was isolated, and the solution was prepared to give free hydroxylamine.

To a flask containing compound 111-1 (100.0 mg, 0.19 mmol) was added a saturation solution of hydroxylamine in methanol (4.0 mL). The mixture was stirred at room temperature for 30 min. It was then adjusted to pH7 using acetic acid. The mixture was concentrated to give a residue and this was washed with water to afford crude product which was purified by column chromatography to afford the product 1 as a white solid (40 mg, 39%). LCMS: 538 [M+1]; ¹H NMR (DMSO-d₆): δ 1.28 (d, J=6.9 Hz, 3H), 4.36 (t, J=5.8 Hz, 1H), 7.15 (m, 3H), 7.36 (s, 1H), 7.57-7.67 (m, 4H), 8.11 (s, 1H), 8.45 (d, J=6.3 Hz, 1H), 8.56 (d, J=7.8 Hz, 1H), 9.33 (s, 1H), 9.56 (s, 1H).

### Example 2

### Preparation of 4-(4-(3-(4-chloro-3-(trifluoromethyl)phenyl)ureido)phenoxy)-N-(3-(hydroxyamino)-3-oxopropyl)picolinamide (Compound 2)

### Step 2a. Methyl 3-(4-(4-(3-(4-chloro-3-(trifluoromethyl)phenyl)ureido)phenoxy)picolinamido)propanoate (Compound 111-2)

The title compound 111-2 was prepared (110 mg, 31%) from compound 110 (300.0 mg, 0.66 mmol) using a procedure similar to that described for compound 111-1 (Example 1): 537 [M+1]⁺.

### Step 2b. 4-(4-(3-(4-Chloro-3-(trifluoromethyl)phenyl)ureido)phenoxy)-N-(3-(hydroxyamino)-3-oxopropyl)picolinamide (Compound 2)

The title compound 2 was prepared as a solid (50 mg, 47%) from compound 111-2 (110.0 mg, 0.20 mmol) using a procedure similar to that described for compound 1 (Example 1): LCMS: 468 [M+1]⁺; ¹H NMR (DMSO-d₆): δ 2.25 (t, J=6.9 Hz, 2H), 3.47 (m, 2H), 7.16 (m, 3H), 7.38 (d, J=2.4, 1H), 7.60-7.70 (m, 4H), 8.15 (s, 1H), 8.50 (d, 1H), 8.78 (t, J=6.3 Hz, 1H), 9.43 (s, 1H), 9.66 (s, 1H), 10.44 (s, 1H).

### Example 3

### Preparation of 4-(4-(3-(4-chloro-3-(trifluoromethyl)phenyl)ureido)phenoxy)-N-(4-(hydroxyamino)-4-oxobutyl)picolinamide (Compound 3)

### Step 3a. Methyl 4-(4-(4-(3-(4-chloro-3-(trifluoromethyl)phenyl)ureido)phenoxy)picolinamido)butanoate (Compound 111-3)

The title compound 111-3 was prepared (95 mg, 26%) from compound 110 (300.0 mg, 0.66 mmol) using a procedure similar to that described for compound 111-1 (Example 1): LCMS: 551 [M+1]⁺.

### Step 3b. 4-(4-(3-(4-Chloro-3-(trifluoromethyl)phenyl)ureido)phenoxy)-N-(4-(hydroxyamino)-4-oxobutyl)picolinamide (Compound 3)

The title compound 3 was prepared as a solid (45 mg, 48%) from compound 111-3 (95 mg, 0.17 mmol) using a procedure similar to that described for compound 1 (Example 1): LCMS: 552 [M+1]⁺; ¹H NMR (DMSO-d₆): δ 1.70-1.77 (m, 2H), 1.96 (t, J=7.2 Hz, 2H), 3.22-3.29 (m, 2H), 7.15-7.19 (m, 3H), 7.37 (d, J=2.7 Hz, 1H), 7.58-7.69 (m, 4H), 8.13 (s, 1H), 8.51 (d, J=6.0 Hz, 1H), 8.70 (s, 1H), 8.88 (t, J=6.0 Hz, 1H), 9.06 (s, 1H), 9.89 (s, 1H), 10.37 (s, 1H).

### Example 4

### Preparation of 4-(4-(3-(4-chloro-3-(trifluoromethyl)phenyl)ureido)phenoxy)-N-(6-(hydroxyamino)-6-oxohexyl)picolinamide (Compound 5)

### Step 4a. Methyl 6-(4-(4-(3-(4-chloro-3-(trifluoromethyl)phenyl)ureido)phenoxy)picolinamido)hexanoate (Compound 111-5)

The title compound 111-5 was prepared (118 mg, 31%) from compound 110 (300.0 mg, 0.66 mmol) using a procedure similar to that described for compound 111-1 (Example 1): LCMS: 579 [M+1]⁺.

### Step 4b. 4-(4-(3-(4-Chloro-3-(trifluoromethyl)phenyl)ureido)phenoxy)-N-(6-(hydroxyamino)-6-oxohexyl)picolinamide (Compound 5)

The title compound 5 was prepared as a solid (50 mg, 62%) from compound 111-5 (80.0 mg, 0.14 mmol) using a procedure similar to that described for compound 1 (Example 1): LCMS: 580 [M+1]⁺; ¹H NMR (DMSO-d₆): δ 1.18-1.26 (m, 2H), 1.43-1.52 (m, 4H), 1.91 (t, J=7.2 Hz, 2H), 3.19-3.23 (m, 2H), 7.11-7.16 (m, 3H), 7.36 (d, J=2.1 Hz, 1H), 7.55-7.66 (m, 4H), 8.09 (d, J=2.4 Hz, 1H), 8.48 (d, J=5.7 Hz, 1H), 8.58 (s, 1H), 8.71 (t, J=6.0 Hz, 1H), 8.10 (s, 1H), 9.23 (s, 1H), 10.26 (s, 1H).

### Example 5

### Preparation of 4-(4-(3-(4-chloro-3-(trifluoromethyl)phenyl)ureido)phenoxy)-N-(7-(hydroxyamino)-7-oxoheptyl)picolinamide (Compound 6)

### Step 5a. Methyl 7-(4-(4-(3-(4-chloro-3-(trifluoromethyl)phenyl)ureido)phenoxy)picolinamido)heptanoate (Compound 111-6)

The title compound 111-6 was prepared (130 mg, 33%) from compound 110 (300.0 mg, 0.66 mmol) using a procedure similar to that described for compound 111-1 (Example 1): LCMS: 593 [M+1].

### Step 5b. 4-(4-(3-(4-Chloro-3-(trifluoromethyl)phenyl)ureido)phenoxy)-N-(7-(hydroxyamino)-7-oxoheptyl)picolinamide (Compound 6)

The title compound 6 was prepared as a solid (62 mg, 75%) from compound 111-6 (80.0 mg, 0.14 mmol) using a procedure similar to that described for compound 1 (Example 1): LCMS: 594 [M+1]; ¹H NMR (DMSO-d⁶): δ 1.16-1.23 (m, 4H), 1.45-1.49 (m, 4H), 1.89-1.94 (m, 2H), 3.20-3.33 (m, 2H), 7.11-7.16 (m, 3H), 7.36 (d, J=2.1 Hz, 1H), 7.55-7.66 (m, 4H), 8.15 (d, J=2.4 Hz, 1H), 8.50 (d, J=5.7 Hz, 1H), 8.66 (s, 1H), 8.78 (t, J=6.0 Hz, 1H), 9.54 (s, 1H), 9.79 (s, 1H), 10.32 (s, 1H).

### Example 6

### Preparation of 4-(4-(3-(4-chloro-3-(trifluoromethyl)phenyl)ureido)phenoxy)-N-(8-(hydroxyamino)-8-oxooctyl)picolinamide (Compound 7)

### Step 6a. Methyl 8-(4-(4-(3-(4-chloro-3-(trifluoromethyl)phenyl)ureido)phenoxy)picolinamido)octanoate (Compound 111-7)

The title compound 111-7 was prepared (140 mg, 35%) from compound 110 (300.0 mg, 0.66 mmol) using a procedure similar to that described for compound 111-1 (Example 1): LCMS: 607 [M+1]⁺.

### Step 6b. 4-(4-(3-(4-Chloro-3-(trifluoromethyl)phenyl)ureido)phenoxy)-N-(8-(hydroxyamino)-8-oxooctyl)picolinamide (Compound 7)

The title compound 7 was prepared as a solid (50 mg, 63%) from compound 111-7 (80.0 mg, 0.13 mmol) using a procedure similar to that described for compound 1 (Example 1): LCMS: 608 [M+1]⁺; ¹H NMR (DMSO-d₆): δ 1.23-1.25 (m, 6H), 1.46-1.51 (m, 4H), 1.89-1.94 (m, 2H), 3.21-3.34 (m, 2H), 7.14-7.19 (m, 3H), 7.36 (d, J=2.1 Hz, 1H), 7.55-7.66 (m, 4H), 8.15 (d, J=2.4 Hz, 1H), 8.50 (d, J=5.7 Hz, 1H), 8.66 (s, 1H), 8.78 (t, J=6.0 Hz, 1H), 9.54 (s, 1H), 9.79 (s, 1H), 10.32 (s, 1H).

### Example 7

### Preparation of 4-(4-(3-(4-chloro-3-(trifluoromethyl)phenyl)ureido)phenoxy)-N-hydroxypicolinamide (Compound 36)

The title compound 36 was prepared as a white solid (30 mg, 29%) from compound 109 (100.0 mg, 0.22 mmol) using a procedure similar to that described for compound 1 (Example 1): LCMS: 467 [M+1]⁺; ¹H NMR (DMSO-d⁶): δ 7.10-7.18 (m, 3H), 7.31 (d, J=2.4, 2H), 7.57-7.67 (m, 4H), 8.10 (s, 1H), 8.45 (d, J=3.3 Hz, 1H), 8.99 (s, 1H), 9.09 (s, 1H), 9.21 (s, 1H), 11.42 (s, 1H).

### Example 8

### Preparation of 1-(4-chloro-3-(trifluoromethyl)phenyl)-3-(4-(2-(5-(hydroxyamino)-5-oxopentanamido)pyridin-4-yloxy)phenyl)urea (Compound 9)

### Step 8a. 1-(4-(2-Aminopyridin-4-yloxy)phenyl)-3-(4-chloro-3-(trifluoromethyl)phenyl)urea (Compound 201)

A mixture of compound 110 (345 mg, 0.8 mmol), DMF (7 mL) and triethyl amine (0.2 mL) was stirred at 60° C. for 1 hour. The mixture was then cooled to 0° C. and DPPA (280 mg, 1.0 mmol) was added. The mixture was stirred overnight. HOAc (3.5 mL) in water (3.5 mL) was added to the mixture. The mixture was heated at 90° C. for 1 hour, and then poured to ice-cold NaOH solution (5.25 g in 140 mL of H₂O). The mixture was extracted with ethyl acetate and washed with water. The organic phase was collected and solvent was removed under reduced pressure. The residue was purified by chromatography on silica gel (mobile phase: ethyl acetate/methanol=4:1) to afford compound 201 as a pale-yellow solid (123 mg, 37.5%). LC-MS: 423 [M+1]⁺, ¹H NMR (DMSO-d₆): δ 2.70 (s, 1H), 2.86 (s, 1H), 5.78 (d, J=2.4 Hz, 1H), 5.88 (s, 1H), 6.10 (m, 1H), 7.02˜7.06 (m, 1H), 7.48˜7.61 (m, 4H), 7.76 (d, J=5.6 Hz, 1H), 8.10 (d, J=2.0 Hz, 1H), 9.40 (s, 1H), 9.76 (s, 1H).

### Step 8b. Methyl 5-(4-(4-(3-(4-chloro-3-(trifluoromethyl)phenyl)ureido)phenoxy)pyridin-2-ylamino)-5-oxopentanoate (Compound 202-9)

A mixture of compound 201 (120 mg, 0.3 mmol), triethylamine (61 mg, 0.6 mmol), Cu powder (38 mg, 0.6 mmol), Zn powder (39 mg, 0.6 mmol) and methylene chloride (2 mL) was heated to 40° C. To above mixture was added methyl 5-chloro-5-oxopentanoate (47 mg, 0.3 mmol). The reaction was monitored by TLC. After the reaction is complete, the solvent was removed under reduced pressure. The residue was purified by chromatography (mobile phase: ethyl acetate/methanol=4:1) on silica gel to afford methyl compound 202-9 as a white solid (160 mg, 96.6%): LC-MS: 551 [M+1]⁺.

### Step 8c. 1-(4-Chloro-3-(trifluoromethyl)phenyl)-3-(4-(2-(5-(hydroxyamino)-5-oxopentanamido)pyridin-4-yloxy)phenyl)urea (Compound 9)

Compound 202-9 (160 mg, 0.3 mmol) was dissolved in freshly prepared NH₂OH methanol solution (1.8 mmol). The mixture was stirred at room temperature overnight. The mixture was then neutralized by HOAc. The solvent was removed in vacuo and the residue was purified by preparative liquid chromatography to give compound 9 as a white solid (20 mg, 12.5%). Melting point: 144˜145° C. LC-MS: 552 [M+1]⁺, ¹H NMR (DMSO-d₆): δ 1.72 (m, 2H), 1.93 (t, J=7.0 Hz, 2H), 2.32 (t, J=7.0 Hz, 2H), 6.6 (m, 1H), 7.10 (m, 2H), 7.52˜7.63 (m, 5H), 8.13 (m, 2H), 8.61 (s, 1H), 8.99 (s, 1H), 9.23 (s, 1H), 10.32 (s, 1H), 10.45 (s, 1H).

### Example 9

### Preparation of 1-(4-chloro-3-(trifluoromethyl)phenyl)-3-(4-(2-(6-(hydroxyamino)-6-oxohexanamido)pyridin-4-yloxy)phenyl)urea (Compound 10)

### Step 9a. Methyl 6-(4-(4-(3-(4-chloro-3-(trifluoromethyl)phenyl)ureido)phenoxy)pyridin-2-ylamino)-6-oxohexanoate (Compound 202-10)

The title compound 202-10 was prepared as a white solid (100 mg, 97%) from compound 201 (77.0 mg, 0.18 mmol), triethyl amine (36 mg, 0.36 mmol), Cu powder (12 mg, 0.18 mmol), Zn powder (12 mg, 0.18 mmol) and Methylene chloride (2 mL) using a procedure similar to that described for compound 202-9 (example 8): LC-MS: 565 [M+1]⁺.

### Step 9b. 1-(4-Chloro-3-(trifluoromethyl)phenyl)-3-(4-(2-(6-(hydroxyamino)-6-oxohexanamido)pyridin-4-yloxy)phenyl)urea (Compound 10)

The title compound 10 was prepared as a white solid (13 mg, 13%) from compound 202-10 (100 mg, 0.18 mmol) and freshly prepared hydroxylamine methanol solution (1.8 mmol) using a procedure similar to that described for compound 9 (example 8): LC-MS: 566 [M+1]⁺, ¹H NMR (DMSO-d₆): δ 1.45 (m, 4H), 1.96 (m, 2H), 2.31 (m, 2H), 6.63 (m, 1H), 7.10 (m, 2H), 7.53 (m, 2H), 7.63 (m, 3H), 8.13 (m, 2H), 8.65 (s, 1H), 9.19 (s, 1H), 9.51 (s, 1H), 10.32 (s, 1H), 10.41 (s, 1H).

### Example 10

### Preparation of 1-(4-chloro-3-(trifluoromethyl)phenyl)-3-(4-(2-(8-(hydroxyamino)-8-oxooctanamido)pyridin-4-yloxy)phenyl)urea (Compound 12)

### Step 10a. Methyl 8-(4-(4-(3-(4-chloro-3-(trifluoromethyl)phenyl)ureido)phenoxy)pyridin-2-ylamino)-8-oxooctanoate (Compound 202-12)

The title compound 202-12 was prepared as a white solid (166 mg, 39.4%) from compound 201 (300 mg, 0.7 mmol), triethyl amine (141 mg, 1.4 mmol), Cu powder (45 mg, 0.7 mmol), Zn powder (45 mg, 0.7 mmol) and methylene chloride (10 mL) using a procedure similar to that described for compound 202-9 (example 8): LC-MS: 593 [M+1]⁺.

### Step 10b. 1-(4-Chloro-3-(trifluoromethyl)phenyl)-3-(4-(2-(8-(hydroxyamino)-8-oxooctanamido)pyridin-4-yloxy)phenyl)urea (Compound 12)

The title compound 12 was prepared as a white solid (25 mg, 15.6%) from compound 202-12 (160 mg, 0.3 mmol) and freshly prepared hydroxylamine methanol solution (1.8 mmol) using a procedure similar to that described for compound 9 (example 8): melting point: 171˜175° C. LC-MS: 594 [M+1]⁺, ¹H NMR (DMSO-d₆): δ 1.21 (s, 4H), 1.47 (m, 4H), 1.90 (t, J=7.5 Hz, 2H), 2.30 (t, J=7.5 Hz, 2H), 6.62 (m, 1H), 7.10 (m, 2H), 7.52 (m, 2H), 7.64 (m, 3H), 8.12 (m, 2H), 8.59 (s, 1H), 8.93 (s, 1H), 9.17 (s, 1H), 10.26 (s, 1H), 10.40 (s, 1H).

### Example 11

### Preparation of 3-((4-(4-(3-(4-chloro-3-(trifluoromethyl)phenyl)ureido)phenoxy)pyridin-2-yl)methylamino)-N-hydroxypropanamide (Compound 13)

### Step 11a. 1-(4-Chloro-3-(trifluoromethyl)phenyl)-3-(4-(2-(hydroxymethyl)pyridin-4-yloxy)phenyl)urea (Compound 301)

AlLiH₄ (0.323 g, 8.5 mmol) was added into a solution of compound 109 (3.3 g, 7.1 mmol) in 30 mL THF under nitrogen. The mixture was stirred at room temperature for 4 h. Then water (0.3 mL), 15% NaOH solution (0.3 mL) and water (0.9 mL) were added into the mixture. The mixture was filtered and concentrated to give crude product which was purified by column chromatography (ethyl acetate:methanol=9:1) to yield compound 301 as a white solid (1.75 g, 47%): LCMS: 438 [M+1]⁺.

### Step 11b. 1-(4-Chloro-3-(trifluoromethyl)phenyl)-3-(4-(2-(chloromethyl)pyridin-4-yloxy)phenyl)urea (Compound 302)

A solution of SOCl₂ (25 mL, 25 mmol) in toluene (22 mL) was cooled to −10° C. Compound 301 (1.0 g, 2.3 mmol) was added to above cold mixture over a range of 0.5 h. The temperature was then increased slowly to 0° C., and the mixture was stirred for 2 h at 0° C. The cold reaction mixture was filtered, and the solid was washed with toluene and ether. The crude product was suspended in water and neutralized with Na₂CO₃. The mixture was stirred for 10 min and filtered. The solid was thoroughly washed with water and dried under reduced pressure to give the title compound 302 as a white yellow solid (0.84 g, 80%): LCMS: 456 [M+1]⁺.

### Step 11c. Ethyl 3-((4-(4-(3-(4-chloro-3-(trifluoromethyl)phenyl)ureido)phenoxy)pyridin-2-yl)methyl amino)propanoate (Compound 303-13)

A solution of ethyl 3-aminopropanoate hydrogen chloride (270 mg, 1.76 mmol) in methanol was neutralized with KOH (66 mg, 1.76 mmol). The mixture was stirred at room for 10 min and methanol was then evaporated. DMF (4 mL) and 302 (200 mg, 0.44 mmol) were added. The mixture was stirred at room temperature for 8 h. DMF was evaporated by reduce pressure to give a residue which was added 30 mL acetate. The mixture was washed with water, dry over anhydrous Na₂SO₄, filtered and concentrated to obtain 303-13 (143 mg, 60.5%) which was used in the next step without purification. LCMS: 537 [M+1]⁺.

### Step 11d. 3-((4-(4-(3-(4-Chloro-3-(trifluoromethyl)phenyl)ureido)phenoxy)pyridin-2-yl)methylamino)-N-hydroxypropanamide (Compound 13)

Preparation of hydroxylamine in methanol: hydrochloride (4.67 g, 67 mmol) was dissolved in methanol (24 mL) to form solution A. Potassium hydroxide (5.61 g, 100 mmol) was dissolved in methanol (14 mL) to form solution B. The solution A was cooled to 0° C., and solution B was added into solution A dropwise. The mixture was stirred for 30 minutes at 0° C., and the precipitate was filtered to afford the solution of hydroxylamine in methanol.

To a flask containing compound 303-13 (143 mg, 0.27 mmol) was added above freshly prepared solution of hydroxylamine in methanol (4.0 mL). The mixture was stirred at room temperature for 30 min. and was adjusted to pH7 using acetic acid. The mixture was concentrated to give a residue which was washed with water and purified by Pre-HPLC to give the title compound 13 as a white solid (64 mg, 45.2%): LCMS: 524 [M+1]⁺; ¹H NMR (DMSO-d₆): δ 2.12 (t, J=6 Hz, 2H), 2.71 (t, J=6 Hz, 2H), 3.72 (s, 2H), 6.73 (d, J=6 Hz, 1H), 6.95 (s, 1H), 7.10 (d, J=9 Hz, 2H), 7.55-7.68 (m, 4H), 8.12 (s, 1H), 8.34 (d, J=6 Hz, 1H), 9.10 (s, 1H), 9.36 (s, 1H).

### Example 12

### Preparation of 6-((4-(4-(3-(4-chloro-3-(trifluoromethyl)phenyl)ureido)phenoxy)pyridin-2-yl)methylamino)-N-hydroxyhexanamide (Compound 16)

### Step 12a. Methyl 6-((4-(4-(3-(4-chloro-3-(trifluoromethyl)phenyl)ureido)phenoxy)pyridine-2-yl)methylamino)hexanoate (Compound 303-16)

The title compound 303-16 was prepared (108 mg, 43%) from compound 302 (200 mg, 0.44 mmol) and methyl 6-aminohexanoate hydrogen chloride (318 mg, 1.76 mmol) using a procedure similar to that described for compound 303-13 (example 11): LCMS: 565 [M+1]⁺.

### Step 12b. 6-((4-(4-(3-(4-Chloro-3-(trifluoromethyl)phenyl)ureido)phenoxy)pyridin-2-yl)methylamino)-N-hydroxyhexanamide (Compound 16)

The title compound 16 was prepared as a white solid (48 mg, 45%) from compound 303-16 (108 mg, 0.19 mmol) using a procedure similar to that described for compound 13 (example 11): LCMS: 566 [M+1]⁺. ¹H NMR (DMSO-d₆): δ 1.20-1.27 (m, 2H), 1.33-1.49 (m, 4H), 2.43-3.48 (m, 2H), 3.72 (s, 2H), 6.74 (d, J=6 Hz, 1H), 6.94 (s, 1H), 7.10 (d, J=9 Hz, 2H), 7.55-7.68 (m, 4H), 8.12 (s, 1H), 8.34 (d, J=6 Hz, 1H), 9.13 (s, 1H), 9.37 (s, 1H), 9.36 (s, 1H).

### Example 13

### Preparation of 7-((4-(4-(3-(4-chloro-3-(trifluoromethyl)phenyl)ureido)phenoxy)pyridin-2-yl)methylamino)-N-hydroxyheptanamide (Compound 17)

### Step 13a. Methyl 7-((4-(4-(3-(4-chloro-3-(trifluoromethyl)phenyl)ureido)phenoxy)pyridine-2-yl)methylamino)heptanoate (Compound 303-17)

The title compound 303-17 was prepared (87 mg, 34%) from compound 302 (200 mg, 0.44 mmol) and methyl 7-aminoheptanoate hydrogen chloride (343 mg, 1.76 mmol) using a procedure similar to that described for compound 303-13 (example 11): LCMS: 579 [M+1]⁺.

### Step 13b. 7-((4-(4-(3-(4-Chloro-3-(trifluoromethyl)phenyl)ureido)phenoxy)pyridin-2-yl)methylamino)-N-hydroxyheptanamide (Compound 17)

The title compound 17 was prepared as a white solid (36 mg, 41%) from compound 303-17 (87 mg, 0.15 mmol) using a procedure similar to that described for compound 13 (example 11): LCMS: 580 [M+1]⁺; ¹H NMR (DMSO-d₆): δ 1.22 (s, 4H), 1.34-1.37 (m, 2H), 1.49 (t, J=9 Hz, 2H), 1.94 (t, J=7.2 Hz, 2H), 2.43-2.48 (m, 2H), 3.72 (s, 2H), 6.75 (d, J=6 Hz, 1H), 6.94 (s, 1H), 7.10 (d, J=9 Hz, 3H), 7.55-7.69 (m, 4 Hz), 8.12 (s, 1H), 8.34 (d, J=6 Hz, 1H), 9.04 (s, 1H), 9.27 (s, 1H), 10.35 (s, 1H).

### Example 14

### Preparation of 8-((4-(4-(3-(4-chloro-3-(trifluoromethyl)phenyl)ureido)phenoxy)pyridin-2-yl)methylamino)-N-hydroxyoctanamide (Compound 18)

### Step 14a. Methyl 8-((4-(4-(3-(4-chloro-3-(trifluoromethyl)phenyl)ureido)phenoxy)pyridine-2-yl)methylamino)octanoate (Compound 303-18)

The title compound 303-18 was prepared (118 mg, 42.9%) from compound 302 (200 mg, 0.44 mmol) and methyl 8-aminooctanoate hydrogen chloride (368 mg, 1.76 mmol) using a procedure similar to that described for compound 303-13 (example 11): LCMS: 593 [M+1]⁺.

### Step 14b. 8-((4-(4-(3-(4-Chloro-3-(trifluoromethyl)phenyl)ureido)phenoxy)pyridin-2-yl)methylamino)-N-hydroxyoctanamide (Compound 18)

The title compound 18 was prepared as a white solid (73 mg, 62%) from compound 303-18 (118 mg, 0.20 mmol) using a procedure similar to that described for compound 13 (example 11): LCMS: 594 [M+1]⁺. ¹H NMR (DMSO-d₆): δ 1.24 (s, 6H), 1.46-1.51 (m, 4H), 1.92 (t, J=9 Hz, 2H), 3.21-3.34 (m, 2H), 7.14-7.19 (m, 3H), 7.37 (d, J=3 Hz, 1H), 7.60-7.70 (m, 4 Hz), 8.14 (s, 1H), 8.50 (d, J=6 Hz, 1H), 8.66 (s, 1H), 8.79 (t, J=6 Hz, 1H), 9.38 (s, 1H), 9.61 (s, 1H), 10.32 (s, 1H).

### Example 15

### Preparation of N¹-((4-(4-(3-(4-chloro-3-(trifluoromethyl)phenyl)ureido)phenoxy)pyridin-2-yl)methyl)-N⁴-hydroxysuccinamide (Compound 19)

### Step 15a. 4-(4-(3-(4-Chloro-3-(trifluoromethyl)phenyl)ureido)phenoxy)picolinamide (Compound 401)

A solution of compound 109 (1.16 g, 2.5 mmol), NH₃ (0.25 g, 15.0 mmol) in MeOH (10 mL) was stirred at room temperature for 6 h. The solvent was removed under reduce pressure and the crude was washed with water to provide compound 401 as a light yellow solid (1.08 g, 96.2%): LCMS: 451 [M+1]⁺.

### Step 15b. 1-(4-(2-(Aminomethyl)pyridin-4-yloxy)phenyl)-3-(4-chloro-3-(trifluoromethyl)phenyl)urea (Compound 402)

A mixture of compound 401 (1.0 g, 2.2 mmol), BH₃ (6 mL, 1 mol/L), THF (10 mL) in sealed tube was stirred for 6 h at 100° C. (oil bath) under nitrogen atmosphere. The mixture was cooled, treated with MeOH (1.5 mL) and concentrated HCl (1.5 mL), stirred for 2 h at 100° C. The reaction mixture was cooled, adjusted to pH10 with Na₂CO₃ (4 mol/L). The solvent was removed under high vacuum to provide crude product 402 as a brown solid (0.6 g, 67.8%): LCMS: 437 [M+1]⁺.

### Step 15c. Methyl 4-((4-(4-(3-(4-chloro-3-(trifluoromethyl)phenyl)ureido)phenoxy)pyridin-2-yl)methylamino)-4-oxobutanoate (Compound 403-19)

A mixture of compound 402 (100 mg, 0.23 mmol), 4-methoxy-4-oxobutanoic acid (36 mg, 0.27 mmol), EDCI (58 mg, 0.30 mmol), HOBt (40 mg, 0.30 mmol), trimethylamine (81 mg, 0.80 mmol) and anhydrous DMF (2 mL) was stirred for 16 h at room temperature. The solvent was removed under high vacuum and the crude purified by column chromatography on silica gel (CH₂Cl₂/MeOH=10/1) to provide target compound 403-19 (78 mg, 62%) as a yellow solid. LCMS: 551 [M+1]⁺.

### Step 15d. N¹-((4-(4-(3-(4-chloro-3-(trifluoromethyl)phenyl)ureido)phenoxy)pyridin-2-yl)methyl)-N⁴-hydroxysuccinamide (Compound 19)

The title compound 19 was prepared as a light yellow solid (63 mg, 81%) from compound 403-19 (78 mg, 0.14 mmol) using a procedure similar to that described for compound 13 (example 11): LCMS: 552 [M+1]⁺; ¹H NMR (DMSO-d₆): δ 2.20 (t, J=6 Hz, 2H), 2.38 (t, J=6 Hz, 2H), 4.28 (d, J=6 Hz, 2H), 6.70 (d, J=3 Hz, 1H), 6.84 (s, 1H), 7.09 (d, J=9 Hz, 2H), 7.55-7.68 (m, 4H), 8.12 (s, 1H), 8.34 (d, J=6 Hz, 2H), 8.44 (s, 1H), 8.69 (s, 1H), 9.13 (s, 1H), 9.37 (s, 1H), 10.38 (s, 1H).

### Example 16

### Preparation of N¹-((4-(4-(3-(4-chloro-3-(trifluoromethyl)phenyl)ureido)phenoxy)pyridin-2-yl)methyl)-N⁵-hydroxyglutaramide (Compound 20)

### Step 16a. Methyl 4-((4-(4-(3-(4-chloro-3-(trifluoromethyl)phenyl)ureido)phenoxy)pyridin-2-yl)methylamino)-4-oxobutanoate (Compound 403-20)

The title compound 403-20 was prepared as a yellow solid (50 mg, 44.3%) from compound 402 (85 mg, 0.20 mmol) and 5-methoxy-5-oxopentanoic acid (35 mg, 0.24 mmol) using a procedure similar to that described for compound 403-19 (example 15): LCMS: 565 [M+1].

### Step 16b. N¹-((4-(4-(3-(4-Chloro-3-(trifluoromethyl)phenyl)ureido)phenoxy)-pyridin-2-yl)methyl)-N⁵-hydroxyglutaramide (Compound 20)

The title compound 20 was prepared as a light yellow solid (40 mg, 88.5%) from compound 403-20 (45 mg, 0.08 mmol) using a procedure similar to that described for compound 13 (example 11): m.p. 161.8˜164.9° C., LCMS: 566 [M+1]⁺; ¹H NMR (DMSO-d₆) δ 1.69 (m, 2H), 1.95 (t, J=7.2 Hz, 2H), 2.12 (t, J=7.5 Hz, 2H), 4.27 (d, J=5.1 Hz, 2H), 6.74 (d, J=3.6 Hz, 2H), 7.07 (d, J=9.0 Hz, 2H), 7.62 (m, 4H), 8.17 (s, 1H), 8.34 (d, J=7.2 Hz, 2H), 9.51 (s, 1H), 10.27 (s, 1H), 10.43 (s, 1H), 10.61 (s, 1H).

### Example 17

### Preparation of N¹-((4-(4-(3-(4-chloro-3(trifluoromethyl)phenyl)ureido)phenoxy)pyridin-2-yl)methyl)-N⁶-hydroxyadipamide (Compound 21)

### Step 17a. 6-((4-(4-(3-(4-Chloro-3-(trifluoromethyl)phenyl)ureido)phenoxy)pyridin-2-yl)methyl amino)-6-oxohexaneperoxoic acid (Compound 403-21)

The title compound 403-21 was prepared as a yellow solid (84 mg, 63%) from compound 402 (100 mg, 0.23 mmol) and 6-methoxy-6-oxohexanoic acid (43 mg, 0.27 mmol) using a procedure similar to that described for compound 403-19 (example 15): LCMS: 581 [M+1]⁺.

### Step 17b. N¹-((4-(4-(3-(4-Chloro-3-(trifluoromethyl)phenyl)ureido)phenoxy)pyridin-2-yl)methyl)-N⁶-hydroxyadipamide (Compound 21)

The title compound 21 was prepared as a light yellow solid (56 mg, 69%) from compound 403-21 (80 mg, 0.14 mmol) using a procedure similar to that described for compound 13 (example 11): LCMS: 582 [M+1]⁺; ¹H NMR (DMSO-d₆): δ 1.45 (s, 4H), 1.94 (t, J=6 Hz, 2H), 2.11 (t, J=6 Hz, 2H), 4.27 (d, J=6 Hz, 2H), 6.74 (s, 2H), 7.10 (d, J=9 Hz, 2H), 7.56-7.69 (m, 4H), 8.12 (s, 1H), 8.34 (d, J=6 Hz, 2H), 8.69 (s, 1H), 9.18 (s, 1H), 9.42 (s, 1H), 10.35 (s, 1H).

### Example 18

### Preparation of N¹-((4-(4-(3-(4-chloro-3(trifluoromethyl)phenyl)ureido)phenoxy)pyridin-2-yl)methyl)-N⁸-hydroxyoctanediamide (Compound 23)

### Step 18a. Methyl 8-((4-(4-(3-(4-chloro-3-(trifluoromethyl)phenyl)ureido)phenoxy)pyridin-2-yl)methyl amino)-8-oxooctanoate (Compound 403-23)

The title compound 403-23 was prepared as a yellow solid (93 mg, 67%) from compound 402 (100 mg, 0.23 mmol) and 8-methoxy-8-oxooctanoic acid (51 mg, 0.27 mmol) using a procedure similar to that described for compound 403-19 (example 15): LCMS: 607 [M+1]⁺.

### Step 18b. N¹-((4-(4-(3-(4-Chloro-3-(trifluoromethyl)phenyl)ureido)phenoxy)pyridin-2-yl)methyl)-N⁸-hydroxyoctanediamide (Compound 23)

The title compound 23 was prepared as a light yellow solid (52 mg, 61%) from compound 403-23 (88 mg, 0.14 mmol) using a procedure similar to that described for compound 13 (example 11): LCMS: 608 [M+1]⁺; ¹H NMR (DMSO-d₆): δ 1.20-1.23 (m, 4H), 1.14-1.45 (s, 4H), 1.93 (t, J=6 Hz, 2H), 2.10 (t, J=6 Hz, 2H), 4.26 (d, J=6 Hz, 2H), 6.72-6.77 (m, 2H), 7.06 (d, J=9 Hz, 2H), 7.56-7.71 (m, 4H), 8.19 (s, 1H), 8.34 (d, J=6 Hz, 2H), 8.69 (s, 1H), 10.44 (s, 1H), 10.76 (s, 1H).

### Biological Assays:

As stated hereinbefore the derivatives defined in the present invention possess anti-proliferation activity. These properties may be assessed, for example, using one or more of the procedures set out below:

(a) An In Vitro Assay which Determines the Ability of a Test Compound to Inhibit a Kinase.

The Raf kinase assay was performed by following the protocol of Raf kinase assay kit (B-Raf, Upstate, catalog#17-359; C-Raf, Upstate, catalog#17-360) with modifications. Briefly, assay buffer, ATP, substrate and Raf kinase were mixed in a 96 well assay plate. The final kinase assay mixture contained 20 mM MOPS, pH7.2, 25 mM β-glycerol phosphate, 5 mM EGTA, 1 mM sodium orthovanadate, 1 mM DTT, 250 μM ATP and 37.5 mM magnesium chloride, 0.1 μg/well of Raf kinase, and 1 μg/well of MEK-1 substrate protein. Assay samples were incubated for 30 min at room temperature. The kinase reaction was stopped by adding EDTA, pH8 to a final concentration of 25 mM. A 10 μl of the reaction sample was spotted onto nitrocellulose filter and dot blot was performed by adding 1 μg/ml of anti-phospho-MEK-1 antibody in the blocking solution (Licor Bioscience, catalogue #927-40000). The nitrocellulose filter was subsequently incubated with secondary IRDye 800CW goat anti-rabbit antibody (Licor Bioscience, catalogue #926-32211) before reading the signal on an Odyssey imager (Licor Bioscience).

The ability of compounds to inhibit VEGFR2 kinase activity was assayed using HTScan™ VEGFR2 Kinase Assay Kits (Cell Signaling Technologies, Danvers, Mass.). VEGFR2 tyrosine kinase was produced using a baculovirus expression system from a construct containing a human VEGFR2 cDNA kinase domain (Asp805-Val1356) (GenBank accession No. AF035121) fragment amino-terminally fused to a GST-HIS6-Thrombin cleavage site. The protein was purified by one-step affinity chromatography using glutathione-agarose. An anti-phosphotyrosine monoclonal antibody, P-Tyr-100, was used to detect phosphorylation of biotinylated substrate peptides (VEGFR2, Biotin-Gastrin Precursor (Tyr87)). Enzymatic activity was tested in 60 mM HEPES, 5 mM MgCl2 5 mM MnCl2 200 μM ATP, 1.25 mM DTT, 3 μM Na3VO4, 1.5 mM peptide, and 50 ng EGF Receptor Kinase. Bound antibody was detected using the DELFIA system (PerkinElmer, Wellesley, Mass.) consisting of DELFIA® Europium-labeled Anti-mouse IgG (PerkinElmer, #AD0124), DELFIA® Enhancement Solution (PerkinElmer, #1244-105), and a DELFIA® Streptavidin coated, 96-well Plate (PerkinElmer, AAAND-0005). Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm.

Test compounds were dissolved in dimethylsulphoxide (DMSO) to give a 20 mM working stock concentration. Each assay was setup as follows: Added 100 μl of 10 mM ATP to 1.25 ml 6 mM substrate peptide. Diluted the mixture with dH₂0 to 2.5 ml to make 2×ATP/substrate cocktail ([ATP]=400 mM, [substrate]=3 mM). Immediately transfer enzyme from −80° C. to ice. Allowed enzyme to thaw on ice. Microcentrifuged briefly at 4° C. to bring liquid to the bottom of the vial. Returned immediately to ice. Added 10 μl of DTT (1.25 mM) to 2.5 ml of 4×HTScan™ Tyrosine Kinase Buffer (240 mM HEPES pH 7.5, 20 mM MgCl₂, 20 mM MnCl, 12 mM NaVO₃) to make DTT/Kinase buffer. Transfer 1.25 ml of DTT/Kinase buffer to enzyme tube to make 4× reaction cocktail ([enzyme]=4 ng/μL in 4× reaction cocktail). Incubated 12.5 μl of the 4× reaction cocktail with 12.5 μl/well of prediluted compound of interest (usually around 10 μM) for 5 minutes at room temperature. Added 25 μl of 2×ATP/substrate cocktail to 25 μg/well preincubated reaction cocktail/compound. Incubated reaction plate at room temperature for 30 minutes. Added 50 μl/well Stop Buffer (50 mM EDTA, pH 8) to stop the reaction. Transferred 25 μl of each reaction and 75 μl dH₂O/well to a 96-well streptavidin-coated plate and incubated at room temperature for 60 minutes. Washed three times with 200 μl/well PBS/T (PBS, 0.05% Tween-20). Diluted primary antibody, Phospho-Tyrosine mAb (P-Tyr-100), 1:1000 in PBS/T with 1% bovine serum albumin (BSA). Added 100 μl/well primary antibody. Incubated at room temperature for 60 minutes. Washed three times with 200 μl/well PBS/T. Diluted Europium labeled anti-mouse IgG 1:500 in PBS/T with 1% BSA. Added 100 μl/well diluted antibody. Incubated at room temperature for 30 minutes. Washed five times with 200 μl/well PBS/T. Added 100 μl/well DELFIA® Enhancement Solution. Incubated at room temperature for 5 minutes. Detected 615 nm fluorescence emission with appropriate Time-Resolved Plate Reader.

(b) An In Vitro Assay which Determines the Ability of a Test Compound to Inhibit HDAC Enzymatic Activity.

HDAC inhibitors were screened using an HDAC fluorimetric assay kit (AK-500, Biomol, Plymouth Meeting, Pa.). Test compounds were dissolved in dimethylsulphoxide (DMSO) to give a 20 mM working stock concentration. Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm. Each assay was setup as follows: Defrosted all kit components and kept on ice until use. Diluted HeLa nuclear extract 1:29 in assay buffer (50 mM Tris/Cl, pH 8.0, 137 mM NaCl, 2.7 mM KCl, 1 mM MgCl2). Prepared dilutions of Trichostatin A (TSA, positive control) and tested compounds in assay buffer (5× of final concentration). Diluted Fluor de Lys™ Substrate in assay buffer to 100 uM (50 fold=2× final). Diluted Fluor de Lys™ developer concentrate 20-fold (e.g. 50 μl plus 950 μl assay buffer) in cold assay buffer. Second, diluted the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Added assay buffer, diluted trichostatin A or test inhibitor to appropriate wells of the microtiter plate. Added diluted HeLa extract or other HDAC sample to all wells except for negative controls. Allowed diluted Fluor de Lys™ Substrate and the samples in the microtiter plate to equilibrate to assay temperature (e.g. 25 or 37° C. Initiated HDAC reactions by adding diluted substrate (25 μl) to each well and mixing thoroughly. Allowed HDAC reactions to proceed for 1 hour and then stopped them by addition of Fluor de Lys™ Developer (50 μl). Incubated plate at room temperature (25° C.) for 10-15 min. Read samples in a microtiter-plate reading fluorimeter capable of excitation at a wavelength in the range 350-380 nm and detection of emitted light in the range 440-460 nm.

The following TABLE 6-B lists compounds representative of the invention and their activity in HDAC, VEGFR2 and RAF assays. In these assays, the following grading was used: I≧10 μM, 10 μM>II>1 μM, 1 μM>III>0.1 μM, and IV≦0.1 μM for IC₅₀.

TABLE 6-B

| Compound | | | | | | |
|---|---|---|---|---|---|---|
| No. | HDAC | B-Raf | C-Raf | VEGFR2 | PDGFRβ | cKit |
| 1 | II | | | | | |
| 2 | II | | | | | |
| 3 | II | | | | | |
| 5 | III | II | II | IV | III | IV |
| 6 | III | | | | | |
| 7 | II | | | III | III | IV |
| 9 | III | | | IV | | |
| 10 | II | | | | | |
| 12 | III | | IV | IV | IV | IV |
| 16 | III | | | | | |
| 17 | III | | | III | | |
| 18 | III | | | III | | |
| 19 | III | | | | | |
| 20 | III | | | IV | | |
| 21 | III | | | IV | III | IV |
| 23 | III | | | IV | III | IV |
| 25 | II | | | | | |
| 26 | II | | | | | |
| 27 | III | | | | | |
| 28 | III | | | | | |
| 31 | II | | | | | |
| 32 | III | | | IV | | |
| 33 | II | | | | | |
| 34 | III | | | | | |
| 36 | I | | | II | | |

TABLE 7-A

| | |
|---|---|
| | (XII) |
| 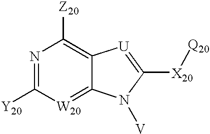 | |

SECTION 7:

| Com- | |
|---|---|
| pound # | Structure |
| 1 | 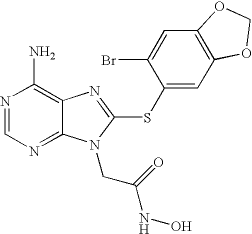 |
| 2 | 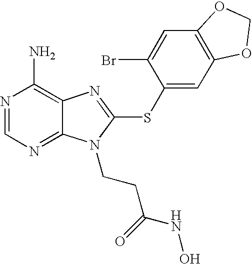 |
| 3 | 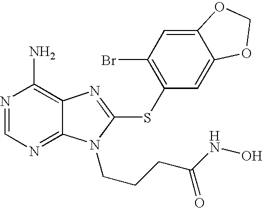 |
| 4 | 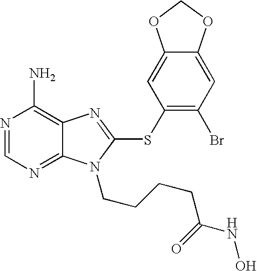 |
| 5 | 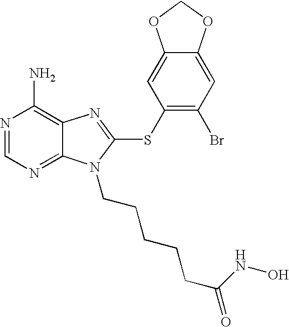 |
| 6 | 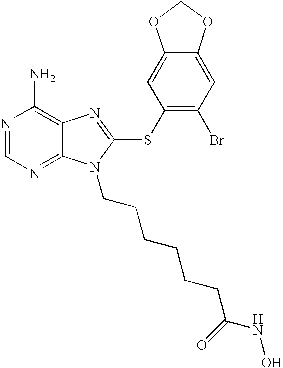 |
| 7 | 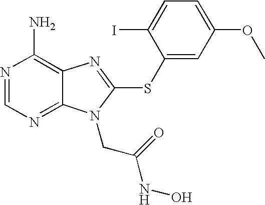 |
| 8 | 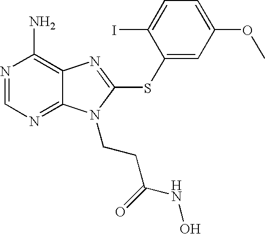 |
| 9 | 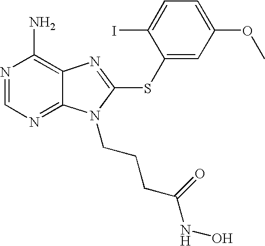 |
| 10 | 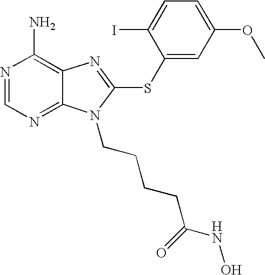 |
| 11 | 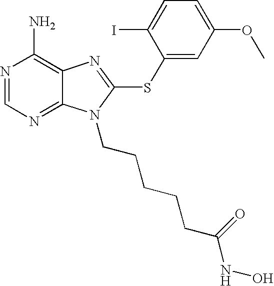 |
| 12 | 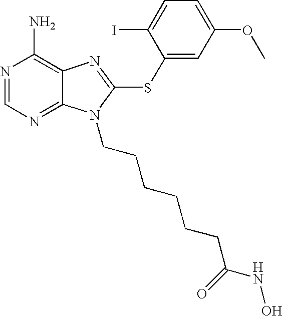 |
| 13 | 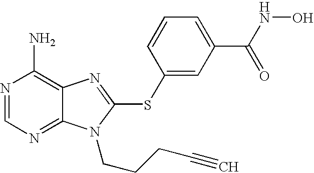 |
| 14 | 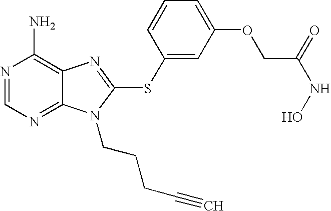 |
| 15 | 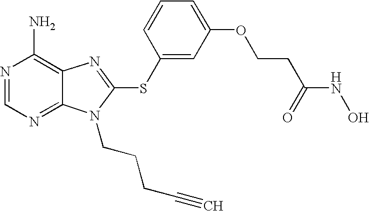 |
| 16 | 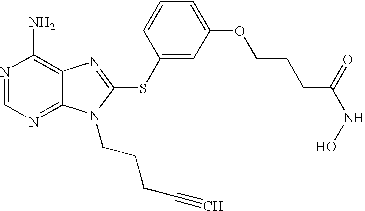 |
| 17 | 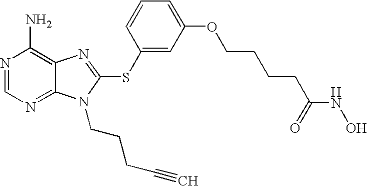 |
| 18 | 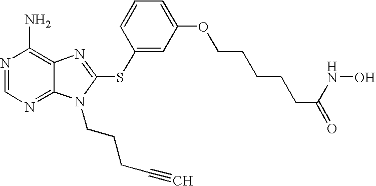 |
| 19 | 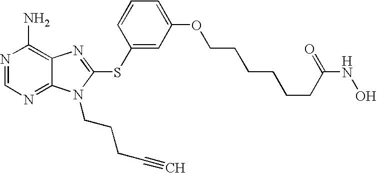 |
| 20 | 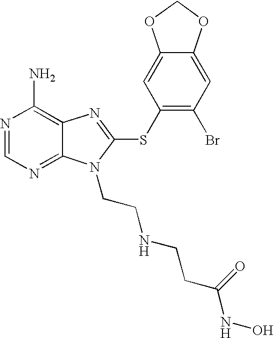 |
| 21 | 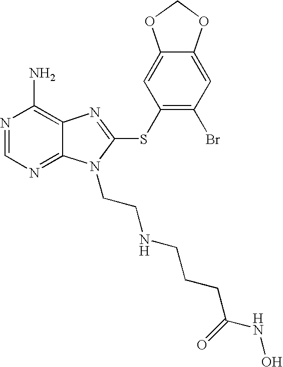 |
| 22 | 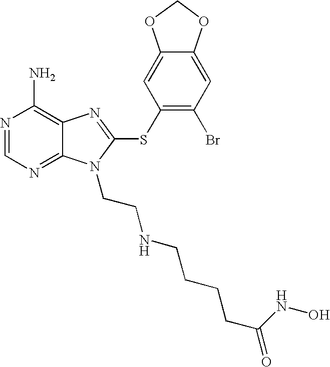 |
| 23 | 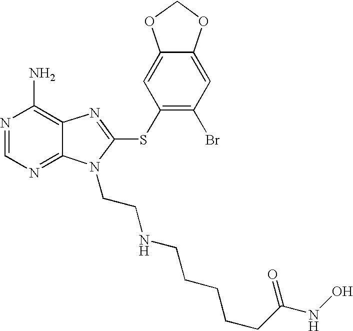 |
| 24 | 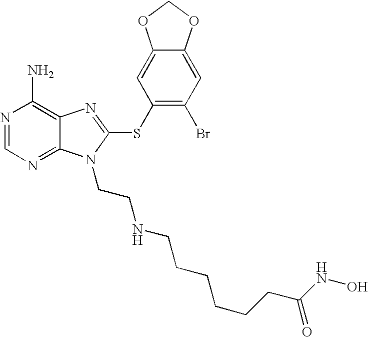 |
| 25 | 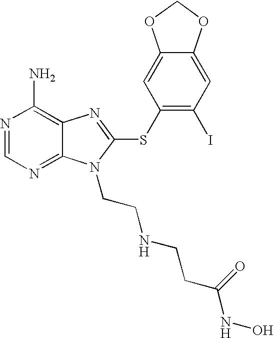 |
| 26 | 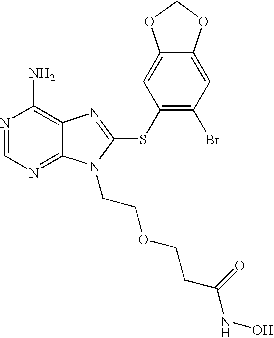 |
| 27 | 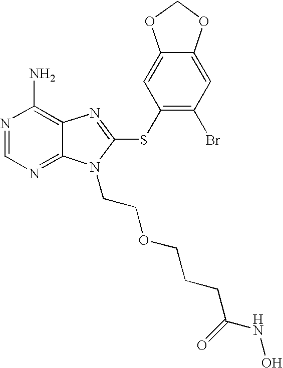 |
| 28 | 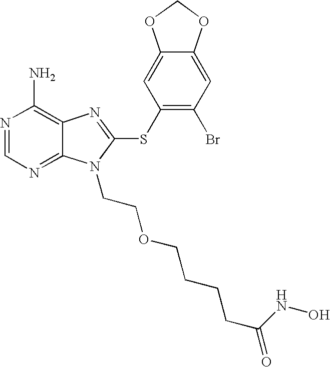 |
| 29 | 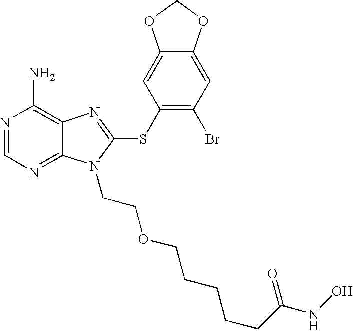 |
| 30 | 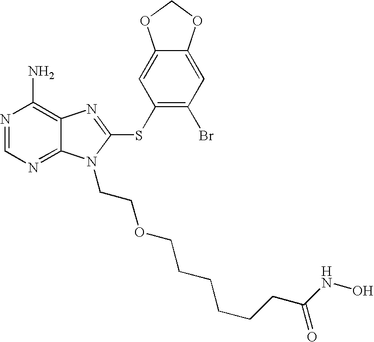 |
| 31 | 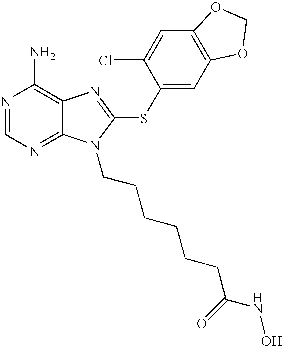 |
| 32 | 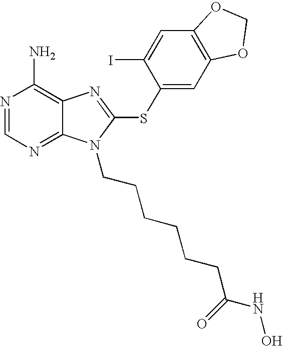 |
| 33 | 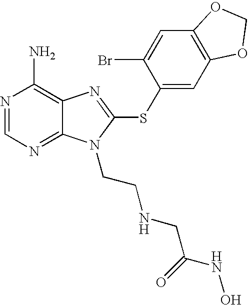 |
| 34 | 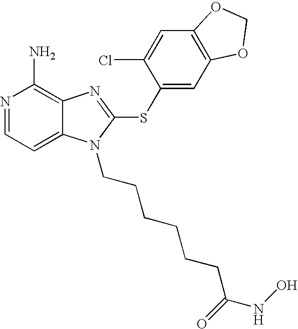 |
| 35 | 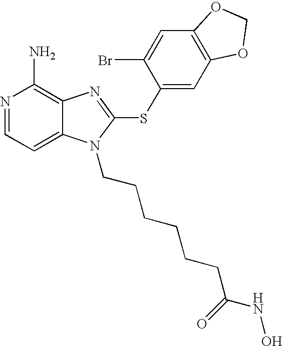 |
| 36 | 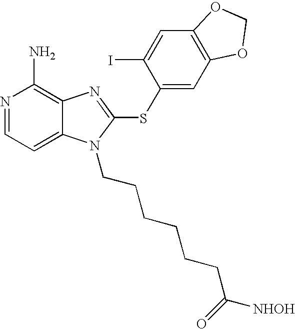 |
| 37 | 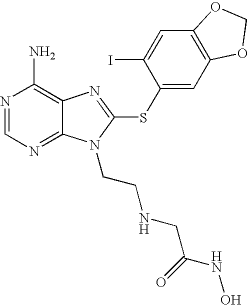 |
| 38 | 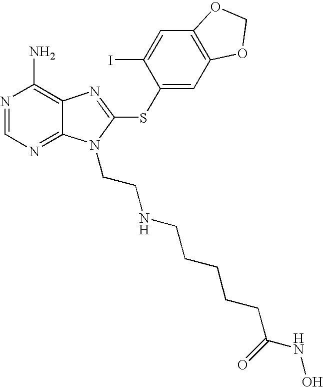 |
| 39 | 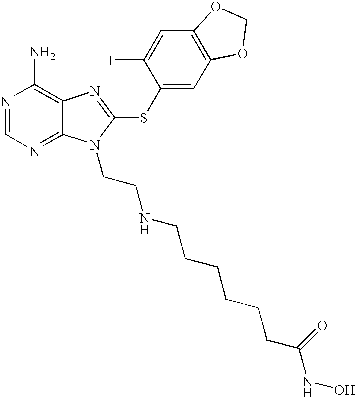 |
| 40 | 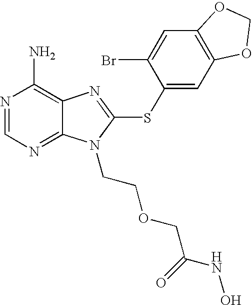 |
| 41 | 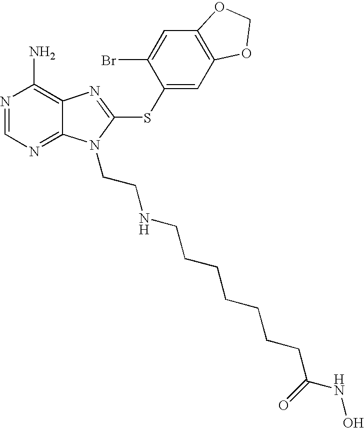 |
| 42 | 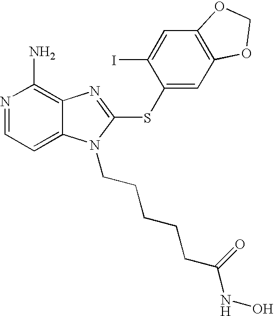 |
| 43 | 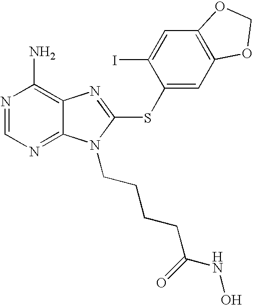 |
| 44 | 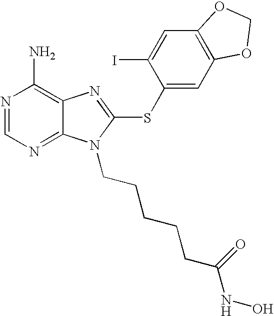 |
| 45 | 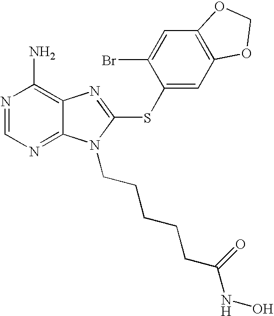 |
| 46 | 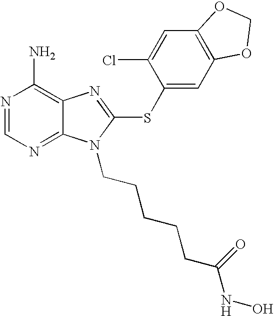 |
| 47 | 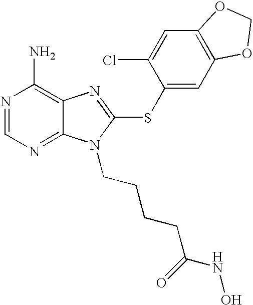 |
| 48 | 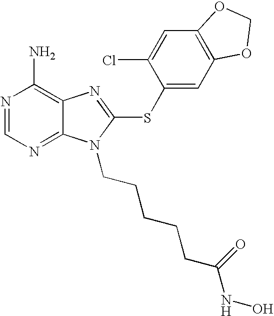 |
| 49 | 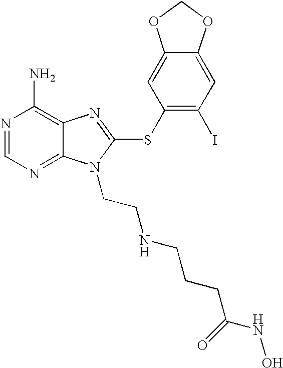 |
| 50 | 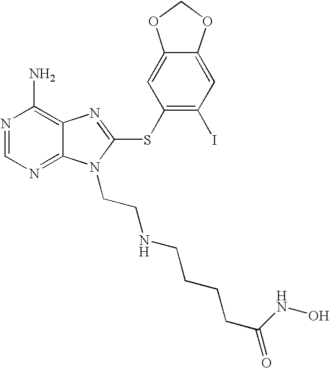 |
| 51 | 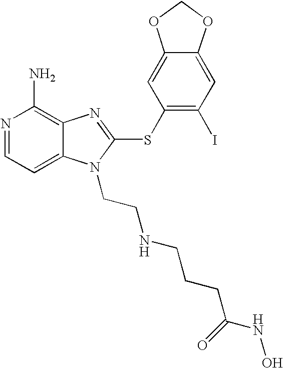 |
| 52 | 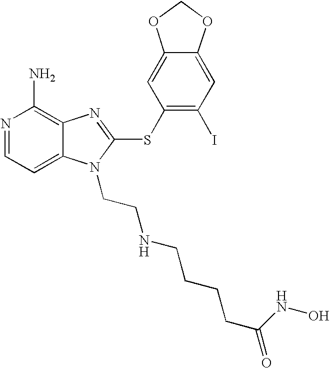 |
| 53 | 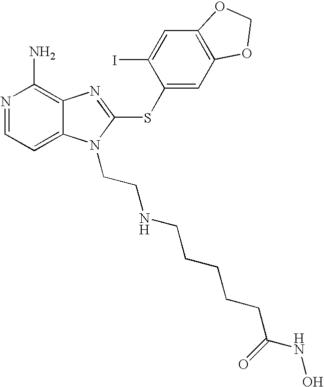 |
| 54 | 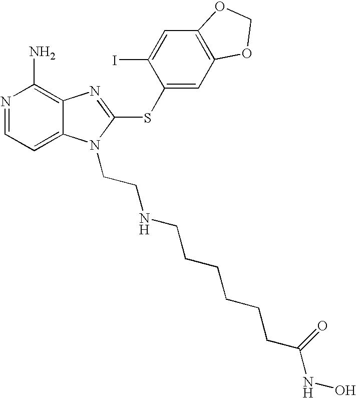 |

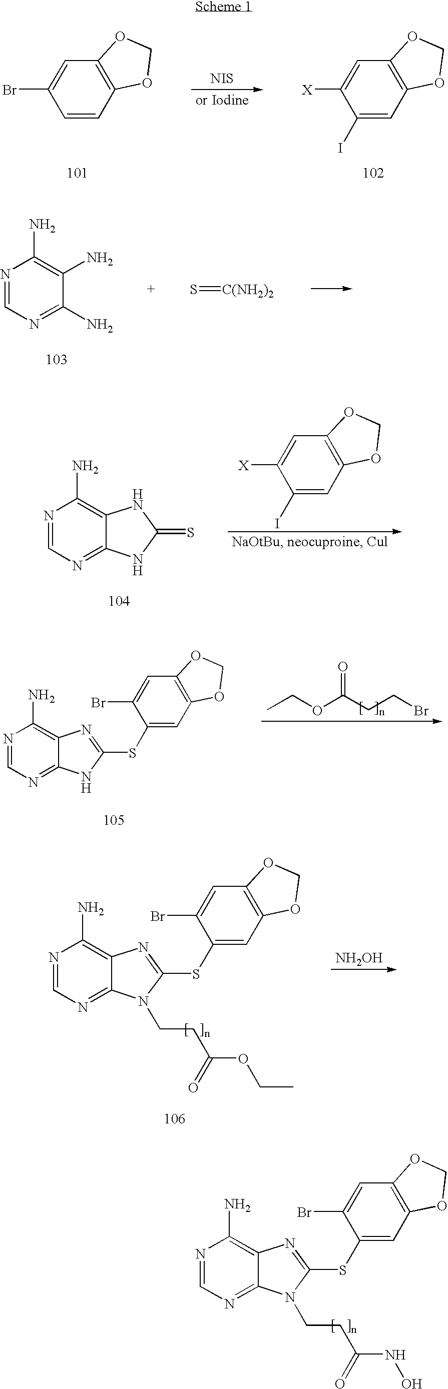

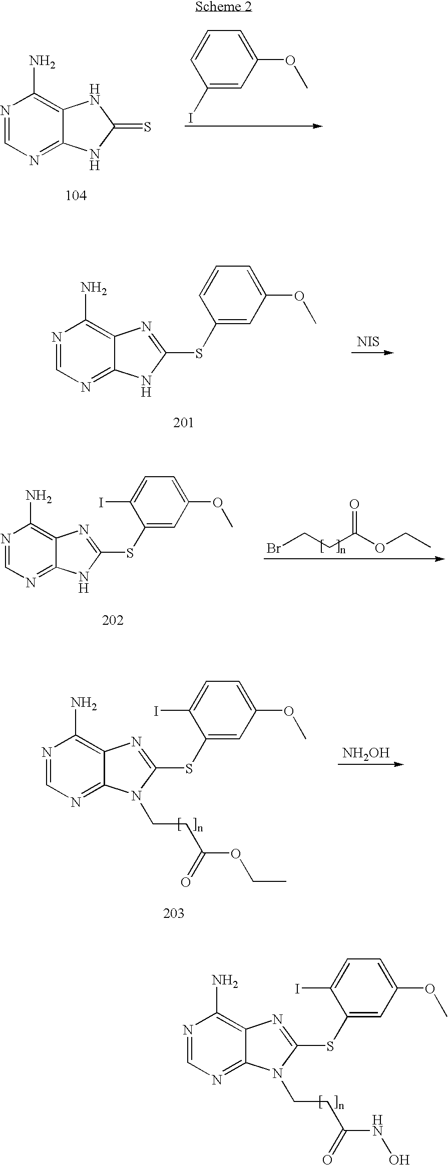

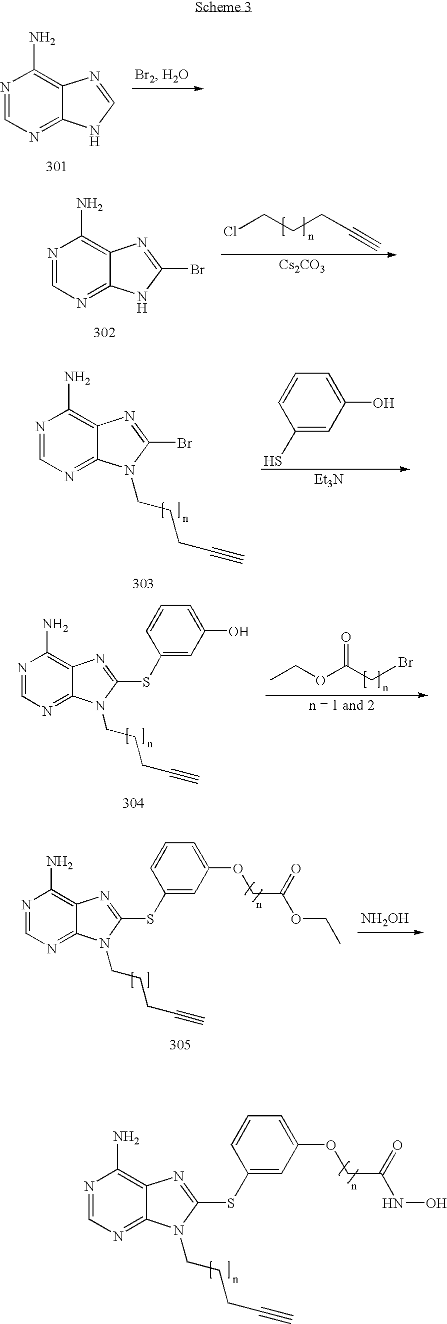

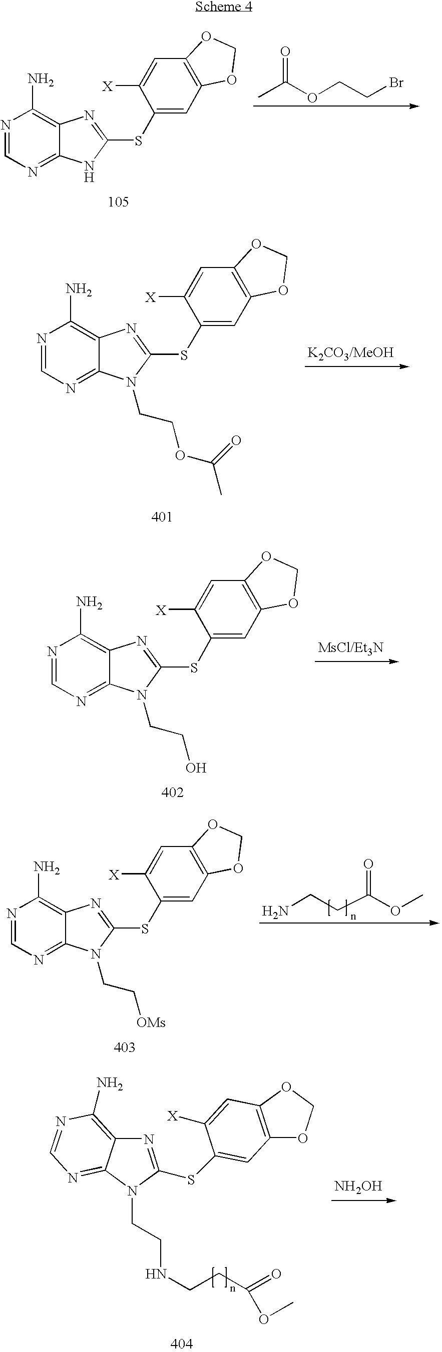

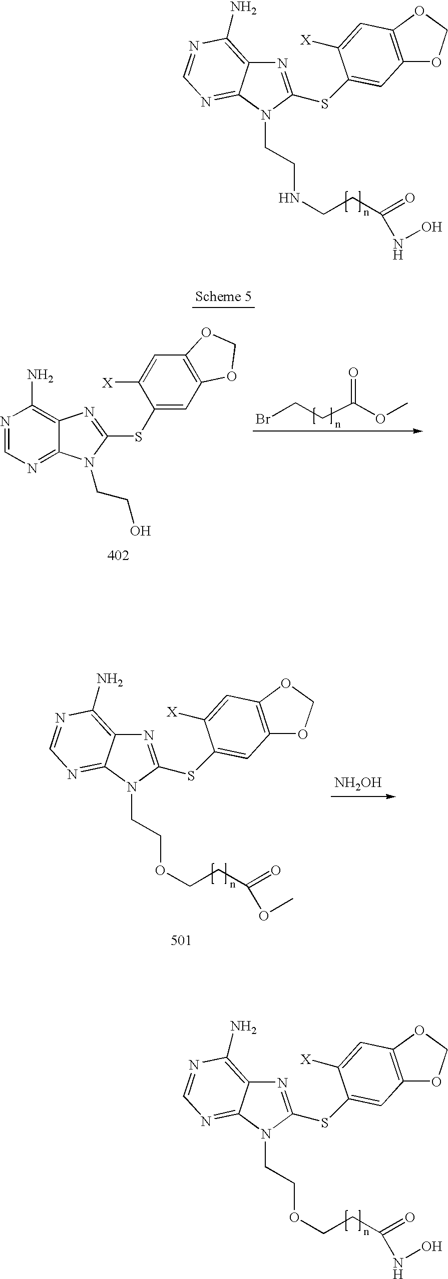

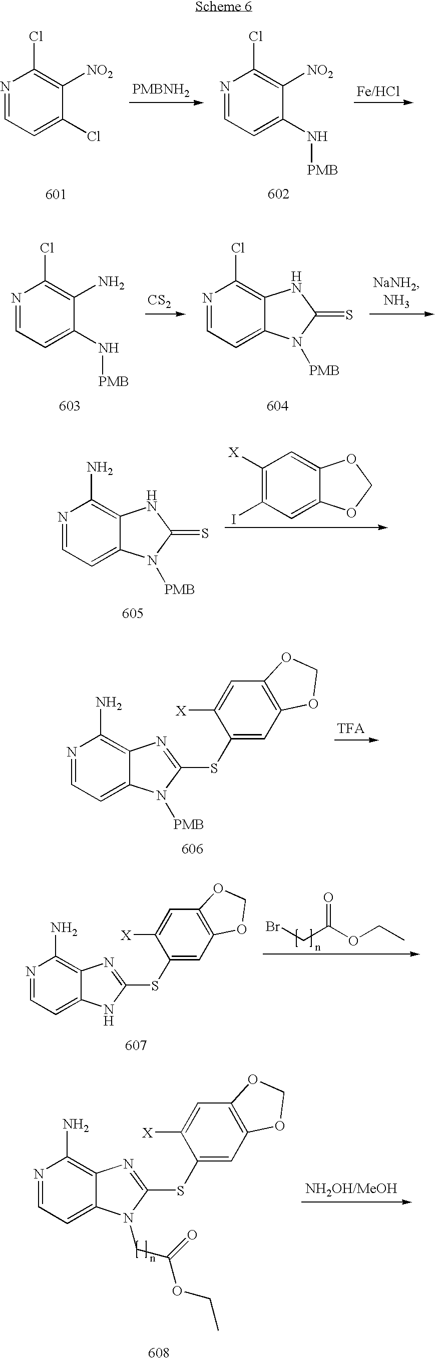

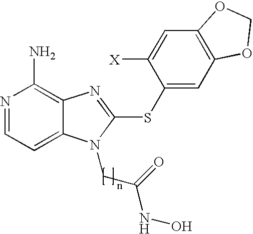

### Example 1

### Preparation of 2-(6-amino-8-(6-bromobenzo[d][1,3]dioxol-5-ylthio)-9H-purin-9-yl)-N-hydroxyacetamide (Compound 1)

### Step 1a. 5-Bromo-6-iodobenzo[d][1,3]dioxole (Compound 102)

A solution of compound 101 (10.0 g, 50.0 mmol), anhydrous acetonitrile (150 mL), TFA (11.4 g, 100.0 mmol) and NIS (33.7 g, 150.0 mmol) was stirred at room temperature for 24 h. The solvent was removed under reduce pressure and the crude purified by column chromatography on silica gel (petroleum) to yield compound 102 as a white solid (18.5 g, 91%): ¹H NMR (DMSO-d₆) δ 5.99 (s, 2H), 7.10 (s, 1H), 7.26 (s, 1H).

### Step 1b. 6-Amino-7H-purine-8(9H)-thione (Compound 104)

A mixture of 4,5,6-triaminopyrimidine sulfate (50.0 g, 223.0 mmol), NaOH (19.7 g, 493.0 mmol) and water (500 mL) was heated to 80° C. until all the solids dissolved. The solution was cooled to 05° C. and the pH was adjusted to 7.0 with 1N HCl, whereupon the free base crystallized as white needles (27.6 g, 99%). A mixture of 4,5,6-triaminopyrimidine 103 (10.0 g, 80.0 mmol), thiourea (18.3 g, 240.0 mmol) in 1,2-dichlorobenzene (60 mL) was stirred for 14 hours at 160° C. Cooled to room temperature and the mixture solidified. Poured out the clear liquid, the solid was triturate and was diluted with brine. The mixture was stirred for 2 hours at room temperature and filtered to obtain crude product. The crude product was washed with brine and ether, dried to give title compound 104 as a light yellow solid (7.35 g, 54.9%). ¹H NMR (DMSO-d₆) δ 6.77 (s, 2H), 8.08 (s, 1H), 12.06 (s, 1H), 13.05 (s, 1H).

### Step 1c. 8-(6-Bromobenzo[d][1,3]dioxol-5-ylthio)-9H-purin-6-amine (Compound 105)

A mixture of compound 104 (5.0 g, 30.0 mmol), compound 102 (14.7 g, 45.0 mmol), neocuproine hydrate (0.625 g, 3.0 mmol), CuI (0.571 g, 3.0 mmol) and NaO-t-Bu (3.5 g, 36.0 mmol) in anhydrous DMF (100 mL) was stirred for 24 hours at 110° C. (oil bath) under nitrogen atmosphere. The solvent was removed under high vacuum and the crude purified by column chromatography on silica gel (CH₂Cl₂/MeOH: 30/1) to provide target compound 105 as a yellow solid (5.3 g, 48.2%): LCMS: 366 [M]; ¹H NMR (DMSO-d₆) δ 6.09 (s, 2H), 7.02 (s, 1H), 7.11 (s, 2H), 7.35 (s, 1H), 8.06 (s, 1H).

### Step 1d. Ethyl 2-(6-amino-8-(6-bromobenzo[d][1,3]dioxol-5-ylthio)-9H-purin-9-yl)acetate (Compound 106-1)

A mixture of compound 105 (1.0 g, 2.73 mmol), Cs₂CO₃ (1.5 g, 4.64 mmol), ethyl 2-bromoacetate (0.685 g, 4.1 mmol) and anhydrous DMF (40 mL) was stirred for 6 hours at room temperature. The solvent was removed under high vacuum and the crude purified by column chromatography on silica gel (CH₂Cl₂/MeOH: 100/1) to provide the title compound 106-1 (0.65 g, 52.6%) as a white solid. LCMS: 452 [M]

### Step 1e. 2-(6-Amino-8-(6-bromobenzo[d][1,3]dioxol-5-ylthio)-9H-purin-9-yl)-N-hydroxyacetamide (Compound 1)

To a stirred solution of hydroxylamine hydrochloride (4.67 g, 67.0 mmol) in methanol (24 mL) at 0° C. was added a solution of potassium hydroxide (5.61 g, 100.0 mmol) in methanol (14 mL). After addition, the mixture was stirred for 30 minutes at 0° C., and was allowed to stand at low temperature. The resulting precipitate was isolated, and the solution was prepared to give free hydroxylamine.

A mixture of compound 106-1 (300 mg, 0.66 mmol) and saturated NH₂OH solution (1.77M, 5 mL) was stirred for 30 minutes at room temperature. The mixture was adjusted to pH 7.0 with AcOH and the solvent was removed. The solid was diluted with water and filtered to provide compound 1 as a white solid (85 mg, 29.2%). m.p. 230° C. (decomp.), LCMS: 439 [M]⁺; ¹H NMR (DMSO-d₆) δ 4.84 (s, 2H), 6.04 (s, 2H), 7.00 (s, 1H), 7.26 (s, 1H), 8.04 (s, 2H), 8.24 (s, 1H), 9.11 (s, 1H), 10.98 (s, 1H).

### Example 2

### Preparation of 4-(6-amino-8-(6-bromobenzo[d][1,3]dioxol-5-ylthio)-9H-purin-9-yl)-N-hydroxybutanamide (Compound 3)

### Step 2a. Ethyl 4-(6-amino-8-(6-bromobenzo[d][1,3]dioxol-5-ylthio)-9H-purin-9-yl)butanoate (Compound 106-3)

The title compound 106-3 was prepared as a white solid (280 mg, 21.4%) from compound 105 (1.0 g, 2.73 mmol), Cs₂CO₃ (1.5 g, 4.64 mmol), ethyl 4-bromobutanoate (800 mg, 4.1 mol) using a procedure similar to that described for compound 106-1 (Example 1): LCMS: 480.34 [M].

### Step 2b. 4-(6-Amino-8-(6-bromobenzo[d][1,3]dioxol-5-ylthio)-9H-purin-9-yl)-N-hydroxybutanamide (Compound 3)

The title compound 3 was prepared as a white solid (207 mg, 76%) from compound 106-3 (280 mg, 0.58 mmol) and NH₂OH solution (1.77M, 5 mL) using a procedure similar to that described for compound 1 (Example 1): m.p. 164.7˜181.0° C., LCMS: 468 [M+1]⁺; ¹H NMR (DMSO-d₆) δ 1.93 (s, 4H), 4.14 (t, 2H, J=6.3 Hz), 6.07 (s, 2H), 6.84 (s, 1H), 7.34 (s, 1H), 7.35 (s, 2H), 8.12 (s, 1H), 8.70 (s, 1H), 10.35 (s, 1H).

### Example 3

### Preparation of 5-(6-amino-8-(6-bromobenzo[d][1,3]dioxol-5-ylthio)-9H-purin-9-yl)-N-hydroxypentanamide (Compound 4)

### Step 3a. Methyl 5-(6-amino-8-(6-bromobenzo[d][1,3]dioxol-5-ylthio)-9H-purin-9-yl)pentanoate (Compound 106-4)

The title compound 106-4 was prepared as a pale yellow solid (463 mg, 35.3%) from compound 105 (1.0 g, 2.73 mmol), Cs₂CO₃ (1.5 g, 4.64 mmol), ethyl 5-bromopentanoate (800 mg, 4.1 mol) using a procedure similar to that described for compound 106-1 (Example 1): LCMS: 480 [M]⁺.

### Step 3b. 5-(6-Amino-8-(6-bromobenzo[d][1,3]dioxol-5-ylthio)-9H-purin-9-yl)-N-hydroxypentanamide (Compound 4)

The title compound 4 was prepared as a white solid (130 mg, 28%) from compound 106-4 (463 mg, 0.96 mmol) and NH₂OH solution (1.77M, 5 mL) using a procedure similar to that described for compound 1 (Example 1): m.p. 191.8˜195.7° C., LCMS: 481 [M]⁺; ¹H NMR (DMSO-d₆) δ 1.43 (q, 2H, J₁=6.9 Hz, J₂=14.7 Hz), 1.68 (m, 2H), 1.94 (t, 2H, J=7.5 Hz), 4.14 (t, 2H, J=6.9 Hz)), 6.10 (s, 2H), 6.86 (s, 1H), 7.37 (s, 1H), 7.39 (s, 2H), 8.15 (s, 1H), 8.67 (s, 1H), 10.33 (s, 1H).

### Example 4

### Preparation of 6-(6-amino-8-(6-bromobenzo[d][1,3]dioxol-5-ylthio)-9H-purin-9-yl)-N-hydroxyhexanamide (Compound 5)

### Step 4a. Ethyl 6-(6-amino-8-(6-bromobenzo[d][1,3]dioxol-5-ylthio)-9H-purin-9-yl)hexanoate (Compound 106-5)

The title compound 106-5 was prepared as a yellow solid (0.35 g, 25.2%) from compound 105 (1.0 g, 2.73 mmol), Cs₂CO₃ (1.5 g, 4.64 mmol), ethyl 6-bromohexanoate (914 mg, 4.1 mol) using a procedure similar to that described for compound 106-1 (Example 1): LCMS: 508 [M]⁺.

### Step 4b. 6-(6-Amino-8-(6-bromobenzo[d][1,3]dioxol-5-ylthio)-9H-purin-9-yl)-N-hydroxyhexanamide (Compound 5)

The title compound 5 was prepared as a pale yellow solid (200 mg, 57.6%) from compound 106-5 (350 mg, 0.7 mmol) and NH₂OH solution (1.77M, 5 mL) using a procedure similar to that described for compound 1 (Example 1): m.p. 159.6˜169° C., LCMS: 496 [M+1]; ¹H NMR (DMSO-d₆) δ 1.18 (q, 2H, J₁=6.3 Hz, J₂=14.7 Hz) 1.48 (m, 2H), 1.65 (m, 2H), 1.90 (t, 2H, J=7.5 Hz), 4.14 (t, 2H, J=6.9 Hz), 6.11 (s, 2H), 6.86 (s, 1H), 7.39 (s, 1H), 7.41 (s, 2H), 8.17 (s, 1H), 8.68 (s, 1H), 10.33 (s, 1H).

### Example 5

### Preparation of 7-(6-amino-8-(6-bromobenzo[d][1,3]dioxol-5-ylthio)-9H-purin-9-yl)-N-hydroxyheptanamide (Compound 6)

### Step 5a. Ethyl 7-(6-amino-8-(6-bromobenzo[d][1,3]dioxol-5-ylthio)-9H-purin-9-yl)heptanoate (Compound 106-6)

The title compound 106-6 was prepared as a yellow solid (542 mg, 43.7%) from compound 105 (1.0 g, 2.73 mmol), Cs₂CO₃ (1.5 g, 4.64 mmol), ethyl 7-bromoheptanoate (972 mg, 4.1 mol) using a procedure similar to that described for compound 106-1 (Example 1): LCMS: 522 [M]⁺.

### Step 5b. 7-(6-Amino-8-(6-bromobenzo[d][1,3]dioxol-5-ylthio)-9H-purin-9-yl)-N-hydroxyheptanamide (Compound 6)

The title compound 6 was prepared as a white solid (130 mg, 24.8%) from compound 106-6 (542 mg, 0.66 mmol) and NH₂OH solution (1.77M, 5 mL) using a procedure similar to that described for compound 1 (Example 1): m.p. 193.9˜193.9° C., LCMS: 511 [M+1]⁺; ¹H NMR (DMSO-d⁶) δ 1.20 (m, 4H), 1.43 (m, 2H), 1.62 (m, 2H), 1.90 (t, 2H, J=7.5 Hz), 4.13 (t, 2H, J=6.9 Hz), 6.10 (s, 2H), 7.00 (s, 1H), 6.83 (s, 1H), 7.37 (s, 1H), 7.42 (s, 2H), 8.16 (s, 1H), 8.65 (s, 1H), 10.32 (s, 1H).

### Example 6

### Preparation of 6-(6-amino-8-(2-iodo-5-methoxyphenylthio)-9H-purin-9-yl)-N-hydroxyhexanamide (Compound 11)

### Step 6a. 8-(3-Methoxyphenylthio)-9H-purin-6-amine (Compound 201)

A mixture of compound 104 (2.0 g, 12 mmol), 1-iodo-3-methoxybenzene (4.21 g, 18 mmol), 1,10-phenanthroline hydrate (0.24 g, 1.2 mmol), CuI (0.23 g, 1.2 mmol) and NaOt-Bu (1.38 g, 14.4 mmol) in anhydrous DMF (20 mL) was stirred for 24 h at 110° C. (oil bath) under nitrogen atmosphere. The solvent was removed under high vacuum and the crude purified by column chromatography on silica gel (CH₂Cl₂/MeOH=30/1) to provide target compound 201 as a yellow solid (0.86 g, 26%): LCMS: 274 [M+1]⁺.

### Step 6b. 8-(2-Iodo-5-methoxyphenylthio)-9H-purin-6-amine (Compound 202)

A mixture of compound 201 (0.69 mg, 2.52 mmol), NIS (3.4 g, 15.12 mmol), trifluoroacetic acid (1.44 g, 12.6 mmol) and acetonitrile (150 mL) was stirred at room temperature for 4 h. The solvent was removed and the residue was suspended in saturated aqueous NaHCO₃ solution, the resulting solid was collected and dried to give compound 202 as a pale yellow solid (810 mg, 80%): LCMS: 400 [M+1]⁺.

### Step 6c. Ethyl 6-(6-amino-8-(2-iodo-5-methoxyphenylthio)-9H-purin-9-yl)hexanoate (Compound 203-11)

A mixture of compound 202 (102 mg, 0.25 mmol), Cs₂CO₃ (98 mg, 0.3 mmol), ethyl 6-bromohexanoate (56 mg, 0.25 mol) and anhydrous DMF (5 mL) was stirred for 2 h at 60° C. The solvent was removed under high vacuum and the crude purified by column chromatography on silica gel (ethyl acetate/petroleum ether=1/2) to give compound 203-11 as a yellow solid (52 mg, 38%). LCMS: 542 [M+1]⁺.

### Step 6d. 6-(6-Amino-8-(2-iodo-5-methoxyphenylthio)-9H-purin-9-yl)-N-hydroxyhexanamide (Compound 11)

To a stirred solution of hydroxylamine hydrochloride (4.67 g, 67 mmol) in methanol (24 mL) at 0° C. was added a solution of potassium hydroxide (5.61 g, 100 mmol) in methanol (14 mL). After addition, the mixture was stirred for 30 minutes at 0° C., and was allowed to stand at low temperature. The resulting precipitate was isolated, and the solution was prepared to give free hydroxylamine.

A mixture of compound 203-11 (50 mg, 0.09 mmol) and freshly prepared NH₂OH/MeOH (1.77 M, 3 mL, 5.3 mmol) was stirred at room temperature for 15 min. The reaction mixture was neutralized with AcOH, and the solvent was removed to give crude product. The crude product was purified by pre-HPLC to give the title compound 11 as a white solid (15 mg, 31%): LCMS: 529 [M+1], ¹H NMR (DMSO-d): δ 1.18 (m, 2H), 1.42 (m, 2H), 1.64 (m, 2H), 1.86 (t, J=6.9 Hz, 2H), 3.62 (s, 3H), 4.12 (t, J=7.2 Hz, 2H), 6.49 (d, J=2.7 Hz, 1H), 6.70 (dd, J₁=3.0 Hz, J₂=8.4 Hz, 1H), 7.51 (s, 2H), 7.78 (d, J=8.1 Hz, 1H), 8.19 (s, 1H), 8.65 (s, 1H), 10.29 (s, 1H).

### Example 7

### Preparation of 7-(6-amino-8-(2-iodo-5-methoxyphenylthio)-9H-purin-9-yl)-N-hydroxyheptanamide (Compound 12)

### Step 7a: Ethyl 7-(6-amino-8-(2-iodo-5-methoxyphenylthio)-9H-purin-9-yl)heptanoate (Compound 203-12)

The title compound 203-12 was prepared as a yellow solid (72 mg, 22%) from compound 202 (239 mg, 0.6 mmol), Cs₂CO₃ (391 mg, 1.2 mmol), ethyl 7-bromoheptanoate (156 mg, 0.66 mol) and anhydrous DMF (5 mL) using a procedure similar to that described for compound 203-11 (Example 6): LCMS: 556 [M+1]⁺.

### Step 7b. 7-(6-Amino-8-(2-iodo-5-methoxyphenylthio)-9H-purin-9-yl)-N-hydroxyheptanamide (Compound 12)

The title compound 12 was prepared as a pale white solid (11 mg, 16%) from compound 203-12 (71 mg, 0.13 mmol) and NH₂OH/MeOH (1.77 M, 3 mL, 5.3 mmol) using a procedure similar to that described for compound 11 (Example 6): LCMS: 543 [M+1]⁻, ¹H NMR (DMSO-d₆); δ 1.16 (m, 4H), 1.37 (m, 2H), 1.61 (m, 2H), 1.87 (t, J=7.8 Hz, 2H), 3.61 (s, 3H), 4.12 (t, J=6.9 Hz, 2H), 6.49 (d, J=3.0 Hz, 1H), 6.70 (dd, J₁=2.7 Hz, J₂=8.7 Hz, 1H), 7.51 (s, 2H), 7.78 (d, J=8.7 Hz, 1H), 8.19 (s, 1H), 8.64 (s, 1H), 10.30 (s, 1H).

### Example 8

### Preparation of 2-(3-(6-amino-9-(pent-4-ynyl)-9H-purin-8-ylthio)phenoxy)-N-hydroxyacetamide (Compound 14)

### Step 8a. 8-Bromo-9H-purin-6-amine (compound 302)

Bromine (9.36 g, 58.5 mmol) was added to H₂O (25 mL) with stirring, then the compound 301 (1.1 g, 8.1 mmol) was added into the solution. The mixture was stirred at room temperature overnight. The excess bromine was removed and the solvent was evaporated to give compound 302 as a light yellow solid (1.28 g, 74%). The crude product was used without further purification: LC-MS: 214 [M+1].

### Step 8b. 8-Bromo-9-(pent-4-ynyl)-9H-purin-6-amine (Compound 303-14)

A mixture of compound 302 (1.7 g, 8.1 mmol), 5-chloropent-1-yne (1.7 g, 16.2 mmol), Cs₂CO₃ (5.8 g, 17.8 mmol) and 25 mL of DMF was heated to 85° C. and stirred overnight. Then DMF was removed in vacuo. The residue was purified by column chromatography (dichloromethane:methanol=40:1) to give compound 303-14 (512 mg, 23%) as a white solid: LC-MS: 280 [M+1]⁺, ¹H NMR (DMSO-d₆) δ 1.91 (m, 2H), 2.22 (m, 2H), 2.79 (t, J=2.4 Hz, 1H), 4.18 (t, J=7.2 Hz, 2H), 7.36 (s, 2H), 8.11 (s, 1H).

### Step 8c. 3-(6-Amino-9-(pent-4-ynyl)-9H-purin-8-ylthio)phenol (Compound 304-14)

3-Mercaptophenol (134 mg, 1.1 mmol) and NH₃.H₂O (60 mg, 3.5 mmol) were dissolved in 2 mL of methanol, the mixture was stirred at 70° C. for 0.5 hour. Then, compound 303-14 (200 mg, 0.7 mmol) in 3 mL of methanol was added into the mixture. The mixture was stirred at 60° C. overnight. The solvent was removed in vacuo, and the residue was purified by column chromatography on silica gel (CH₂Cl₂: MeOH=40:1) to give compound 304-14 (170 mg, 74%) as a white solid. LC-MS: 326 [M+1]⁺, ¹H NMR (DMSO-d₆); δ 1.80 (m, 2H), 2.22 (m, 2H), 2.76 (t, J=2.4 Hz, 1H), 4.18 (t, J=7.2 Hz, 2H), 6.59˜6.75 (m, 3H), 7.14 (t, J=7.5 Hz, 1H), 7.44 (b, 2H), 8.15 (s, 1H), 9.66 (s, 1H).

### Step 8d. Methyl 2-(3-(6-amino-9-(pent-4-ynyl)-9H-purin-8-ylthio)phenoxy)acetate (Compound 305-14)

A mixture of compound 304-14 (120 mg, 0.37 mmol), K₂CO₃ (153 mg, 1.1 mmol) and ethyl 2-bromoacetate (92 mg, 0.55 mmol) was dissolved in 5 mL of DMF. The mixture was heated to 70° C. and stirred for 4 hours. The solvent was removed in vacuo and the residue was purified by column chromatography on silica gel (CH₂Cl₂: MeOH=20:1) to give compound 305-14 as a white solid (86 mg, 59%): LC-MS: 398 [M+1]⁺.

### Step 8e. 2-(3-(6-Amino-9-(pent-4-ynyl)-9H-purin-8-ylthio)phenoxy)-N-hydroxyacetamide (Compound 14)

The title compound 14 was prepared as a white solid (50 mg, 57%) from compound 305-14 (86 mg, 0.22 mmol) using a procedure similar to that described for compound 11 (Example 6): m.p. 165˜166° C., LC-MS: 399 [M+1]⁺, ¹H NMR (DMSO-d₆): δ 1.84 (m, 2H), 2.15 (m, 2H), 2.78 (t, J=2.4 Hz, 1H), 4.19 (t, J=7.2 Hz, 2H), 4.44 (s, 2H), 6.84˜6.96 (m, 3H), 7.26 (m, 1H), 7.43 (b, 2H), 8.15 (s, 1H), 8.96 (s, 1H), 10.81 (s, 1H).

### Example 9

### Preparation of 4-(3-(6-amino-9-(pent-4-ynyl)-9H-purin-8-ylthio)phenoxy)-N-hydroxybutanamide (Compound 16)

### Step 9a. Ethyl 4-(3-(6-amino-9-(pent-4-ynyl)-9H-purin-8-ylthio)phenoxy)butanoate (Compound 305-16)

The title compound 305-16 was prepared as a white solid (120 mg, 64%) from compound 304 (135 mg, 0.42 mmol), K₂CO₃ (165 mg, 1.2 mmol) and ethyl 4-bromobutanoate (123 mg, 0.63 mmol) using a procedure similar to that described for compound 305-14 (Example 8): LC-MS: 440 [M+1]⁺.

### Step 9b. 4-(3-(6-Amino-9-(pent-4-ynyl)-9H-purin-8-ylthio)phenoxy)-N-hydroxybutanamide (Compound 16)

The title compound 16 was prepared as a white solid (50 mg, 48%) from compound 305-16 (110 mg, 0.25 mmol) using a procedure similar to that described for compound 11 (Example 6): m.p. 159˜162° C., LC-MS: 427 [M+1]⁺, ¹H NMR (DMSO-d₆): δ 1.83 (m, 4H), 2.04˜2.18 (m, 4H), 2.77 (t, J=2.4 Hz, 1H), 3.90 (t, J=6.0 Hz, 2H), 4.20 (t, J=8.1 Hz, 2H), 6.80˜6.90 (m, 3H), 7.24 (m, 1H), 7.42 (b, 2H), 8.14 (s, 1H), 8.68 (s, 1H), 10.37 (s, 1H).

### Example 10

### Preparation of 6-(3-(6-amino-9-(pent-4-ynyl)-9H-purin-8-ylthio)phenoxy)-N-hydroxyhexanamide (Compound 18)

### Step 10a. Ethyl 6-(3-(6-amino-9-(pent-4-ynyl)-9H-purin-8-ylthio)phenoxy)hexanoate (Compound 305-18)

The title compound 305-18 was prepared as a white solid (238 mg, 85.4%) from compound 304 (194 mg, 0.60 mmol), K₂CO₃ (247 mg, 1.8 mmol) and ethyl 6-bromohexanoate (143 mg, 0.89 mmol) using a procedure similar to that described for compound 305-14 (Example 8): LC-MS: 468 [M+1]⁺.

### Step 10b. 6-(3-(6-Amino-9-(pent-4-ynyl)-9H-purin-8-ylthio)phenoxy)-N-hydroxyhexanamide (Compound 18)

The title compound 18 was prepared as a white solid (50 mg, 45.8%) from compound 305-18 (110 mg, 0.24 mmol) using a procedure similar to that described for compound 11 (Example 6): m.p. 169.1˜172.1° C., LC-MS: 455 [M+1], ¹H NMR (DMSO-d₆): δ 1.33 (m, 2H), 1.49 (m, 2H), 1.63 (m, 2H), 1.81 (m, 2H), 1.95 (t, J=7.2 Hz, 2H), 2.17 (m, 2H), 2.81 (t, J=2.4 Hz, 1H), 3.89 (t, J=6.0 Hz, 2H), 4.22 (t, J=7.5 Hz, 2H), 6.82˜6.89 (m, 3H), 7.24 (m, 1H), 7.48 (b, 2H), 8.16 (s, 1H), 8.69 (s, 1H), 10.35 (s, 1H).

### Example 11

### Preparation of 3-(2-(6-amino-8-(6-bromobenzo[d][1,3]dioxol-5-ylthio)-9H-purin-9-yl)ethylamino)-N-hydroxypropanamide (Compound 20)

### Step 11a. 2-(6-Amino-8-(6-bromobenzo[d][1,3]dioxol-5-ylthio)-9H-purin-9-yl)ethyl acetate (Compound 401-20)

A mixture of compound 105-1 (8.66 g, 23.65 mmol), Cs₂CO₃ (11.53 g, 35.47 mmol), 2-bromoethyl acetate (5.92 g, 35.47 mmol) and anhydrous DMF (150 mL) was stirred for 2 h at 50° C. The solvent was removed under high vacuum and the crude purified by column chromatography on silica gel (CH₂Cl₂/MeOH=60/1) to provide target compound 401-20 as a pale yellow solid (7.0 g, 65.4%): LCMS: 452 [M+1]⁺.

### Step 11b. 2-(6-Amino-8-(6-bromobenzo[d][1,3]dioxol-5-ylthio)-9H-purin-9-yl)ethanol (compound 402-20)

A suspension of compound 401-20 (4.0 g, 8.84 mmol) in MeOH (80 mL) was treated with K₂CO₃ (3.67 g, 26.53 mmol) at 50° C. for 1 h. The reaction was filtered and concentrated to afforded the title compound 402-20 as a pale white solid (1.3 g, 35.7%): LCMS: 410 [M+1]⁺; ¹H NMR (DMSO-d₆): δ 3.72 (t, 2H, J=5.4 Hz), 4.28 (t, 2H, J=5.4 Hz), 5.02 (t, 1H, J=5.4 Hz), 6.10 (s, 2H), 6.90 (s, 1H), 7.35 (s, 3H), 8.16 (s, 1H).

### Step 11c. 2-(6-Amino-8-(6-bromobenzo[d][1,3]dioxol-5-ylthio)-9H-purin-9-yl)ethyl methanesulfonate (Compound 403-20)

The compound 402-20 (0.6 g, 1.46 mmol) was dissolved in hot anhydrous dioxane (35 mL). The solution was cooled to 45° C. and was treated with NEt₃ (0.61 mL, 4.39 mmol) and MsCl (251.2 mg, 2.2 mmol) for 20 min. The mixture was concentrated and purified by column chromatography on silica gel (CH₂Cl₂/MeOH=60/1) to provide compound 403-20 as a pale yellow solid (0.68 g, 95.5%): LCMS: 487 [M+1].

### Step 11d. Methyl 3-(2-(6-amino-8-(6-bromobenzo[d][1,3]dioxol-5-ylthio)-9H-purin-9-yl)ethylamino)propanoate (Compound 404-20)

Methyl 3-aminopropanoate hydrochloride (494.5 mg, 3.54 mmol) was dissolved in DMF (4.8 mL) and NEt₃ (0.74 mL, 5.31 mmol) was then added to the above solution. The mixture was stirred for 0.5 h at 0° C. and then compound 403-20 (173 mg, 0.35 mmol) was added. The reaction mixture was stirred for 12 h at 80° C. The DMF was removed under high vacuum and the crude product purified by column chromatography on silica gel (CH₂Cl₂/MeOH=50/1) to provide target compound 404-20 as a viscous yellow solid (121 mg, 69%): LCMS: 495 [M+1]⁺.

### Step 11e. 3-(2-(6-Amino-8-(6-bromobenzo[d][1,3]dioxol-5-ylthio)-9H-purin-9-yl)ethylamino)-N-hydroxypropanamide (Compound 20)

The title compound 20 was prepared as a pale white solid (33 mg, 16.5%) from compound 404-20 (200 mg, 0.40 mmol) and freshly prepared NH₂OH methanol solution (1.77 M, 10 mL) using a procedure similar to that described for compound 11 (Example 6): LCMS: 496 [M+1]⁺; ¹H NMR (DMSO-d₆): δ 2.05 (t, 2H, J=6.9 Hz), 2.69 (t, 2H, J=6.9 Hz), 2.83 (t, 2H, J=6.3 Hz), 4.22 (t, 2H, J=6.3 Hz), 6.10 (s, 2H), 6.88 (s, 1H), 7.36 (s, 1H), 7.37 (s, 2H), 8.16 (s, 1H).

### Example 12

### Preparation of 6-(2-(6-amino-8-(6-bromobenzo[d][1,3]dioxol-5-ylthio)-9H-purin-9-yl)ethylamino)-N-hydroxyhexanamide (Compound 23)

### Step 12a. Methyl 6-(2-(6-amino-8-(6-bromobenzo[d][1,3]dioxol-5-ylthio)-9H-purin-9-yl)ethylamino)hexanoate (Compound 404-23)

The title compound 404-23 was prepared as a viscous yellow solid (117 mg, 23.6%) from compound 403-20 (450 mg, 0.92 mmol), methyl 6-aminohexanoate hydrochloride (1.67 g, 9.21 mmol) and KOH (0.52 g, 9.21 mmol) in MeOH (1.5 mL) using a procedure similar to that described for compound 404-20 (Example 11): LCMS: 537 [M+1]⁺.

### Example 12b

### 6-(2-(6-Amino-8-(6-bromobenzo[d][1,3]dioxol-5-ylthio)-9H-purin-9-yl)ethylamino)-N-hydroxyhexanamide (Compound 23)

The title compound 23 was prepared as a pale white solid (22 mg, 18.8%) from compound 404-23 (117 mg, 0.22 mmol) and freshly prepared NH₂OH methanol solution (1.77 M, 4 mL) using a procedure similar to that described for compound 11 (Example 6): LCMS: 538 [M+1]⁺; ¹H NMR (DMSO-d₆): δ 1.26 (m, 4H), 1.43 (m, 2H), 1.70 (s, 1H), 1.90 (t, 2H, J=7.2 Hz), 2.44 (t, 2H, J=7.2 Hz), 2.81 (t, 2H, J=6.0 Hz), 4.22 (t, 2H, J=6.0 Hz), 6.08 (s, 2H), 6.84 (s, 1H), 7.34 (s, 1H), 7.35 (s, 2H), 8.15 (s, 1H), 8.65 (s, 1H), 10.31 (s, 1H).

### Example 13

### Preparation of 7-(2-(6-amino-8-(6-bromobenzo[d][1,3]dioxol-5-ylthio)-9H-purin-9-yl)ethylamino)-N-hydroxyheptanamide (Compound 24)

### Step 13a. Ethyl 7-(2-(6-amino-8-(6-bromobenzo[d][1,3]dioxol-5-ylthio)-9H-purin-9-yl)ethylamino)heptanoate (Compound 404-24)

The title compound 404-24 was prepared as a viscous yellow solid (118 mg, 27%) from compound 403-20 (373 mg, 0.76 mmol), ethyl 7-aminoheptanoate hydrochloride (1.6 g, 7.6 mmol) and KOH (0.43 g, 7.6 mmol) in MeOH (1.0 mL) using a procedure similar to that described for compound 404-20 (Example 11): LCMS: 565 [M+1]⁺.

### Step 13b. 7-(2-(6-Amino-8-(6-bromobenzo[d][1,3]dioxol-5-ylthio)-9H-purin-9-yl)ethylamino)-N-hydroxyheptanamide (Compound 24)

The title compound 24 was prepared as a pale white solid (47 mg, 40.5%) from compound 404-24 (118 mg, 0.21 mmol) and freshly prepared NH₂OH methanol solution (1.77 M, 4 mL) using a procedure similar to that described for compound 11 (Example 6): m.p. 193˜197° C. LCMS: 552 [M+1]⁺; ¹H NMR (DMSO-d₆): δ 1.17 (m, 6H), 1.44 (m, 2H), 1.91 (t, 2H, J=7.2 Hz), 2.43 (t, 2H, J=7.2 Hz), 2.82 (t, 2H, J=6.0 Hz), 4.22 (t, 2H, J=6.0 Hz), 6.08 (s, 2H), 6.83 (s, 1H), 7.34 (s, 1H), 7.36 (s, 2H), 8.15 (s, 1H), 8.65 (s, 1H), 10.31 (s, 1H).

### Example 14

### Preparation of 6-(2-(6-amino-8-(6-iodobenzo[d][1,3]dioxol-5-ylthio)-9H-purin-9-yl)ethylamino)-N-hydroxyhexanamide (Compound 38)

### Step 14a. 8-(6-Iodobenzo[d][1,3]dioxol-5-ylthio)-9H-purin-6-amine (compound 105-38)

A mixture of compound 104 (0.8 g, 4.78 mmol), 5,6-diiodobenzo[d][1,3]dioxole (2.68 g, 7.18 mmol), neocuproine hydrate (0.10 g, 0.48 mmol), CuI (0.091 g, 0.48 mmol) and NaO-t-Bu (0.55 g, 5.74 mmol) in anhydrous DMF (40 mL) was stirred for 24 h at 110° C. (oil bath) under nitrogen atmosphere. The solvent was removed under high vacuum and the crude purified by column chromatography on silica gel (CH₂Cl₂/MeOH=30/1) to provide target compound 105-38 as a yellow solid (0.35 mg, 17.6%): LCMS: 414 [M+1]; ¹H NMR (DMSO-d₆): δ 6.09 (s, 2H), 7.01 (s, 1H), 7.22 (s, 2H), 7.51 (s, 1H), 8.08 (s, 1H), 13.20 (s, 1H).

### Step 14b. 2-(6-Amino-8-(6-iodobenzo[d][1,3]dioxol-5-ylthio)-9H-purin-9-yl)ethyl acetate (Compound 401-38)

A mixture of compound 105-38 (3.89 g, 9.41 mmol), Cs₂CO₃ (3.68 g, 11.3 mmol), 2-bromoethyl acetate (1.89 g, 11.3 mmol) and anhydrous DMF (50 mL) was stirred for 2 h at 50° C. The solvent was removed under high vacuum and the crude purified by column chromatography on silica gel (CH₂Cl₂/MeOH=60/1) to provide target compound 401-38 as a pale yellow solid (2.95 g, 62.8%): LCMS: 500 [M+1]⁺.

### Step 14c. 2-(6-Amino-8-(6-iodobenzo[d][1,3]dioxol-5-ylthio)-9H-purin-9-yl)ethanol (Compound 402-38)

A suspension of compound 401-38 (2.95 g, 5.91 mmol) in MeOH (70 mL) was treated with K₂CO₃ (0.98 g, 7.1 mmol) at 50° C. for 1 h. The reaction was filtered and concentrated to afforded the title compound 402-38 as a pale white solid (1.33 g, 49.3%): LCMS: 458 [M+1]⁺; ¹H NMR (DMSO-d₆): δ 3.72 (t, 2H, J=5.4 Hz), 4.27 (t, 2H, J=5.4 Hz), 5.02 (t, 1H, J=5.4 Hz), 6.07 (s, 2H), 6.88 (s, 1H), 7.34 (s, 2H), 7.47 (s, 1H), 8.15 (s, 1H).

### Step 14d. 2-(6-Amino-8-(6-iodobenzo[d][1,3]dioxol-5-ylthio)-9H-purin-9-yl)ethyl methanesulfonate (Compound 403-38)

The compound 402-38 (0.52 g, 1.13 mmol) was dissolved in hot anhydrous dioxane (25 mL). The solution was cooled to 45° C. and was treated with NEt₃ (0.47 mL, 3.39 mmol) and MsCl (194 mg, 1.70 mmol) for 20 min. The mixture was concentrated and purified by column chromatography on silica gel (CH₂Cl₂/MeOH=60/1) to provide compound 403-38 as a pale yellow solid (585 mg, 96.7%): LCMS: 536 [M+1]⁺.

### Step 14e. Methyl 6-(2-(6-amino-8-(6-iodobenzo[d][1,3]dioxol-5-ylthio)-9H-purin-9-yl)ethylamino)hexanoate (Compound 404-38)

A solution of KOH (785 mg, 14 mmol) in MeOH (4 ml) was added dropwise into a solution of methyl 6-aminohexanoate hydrochloride (2543 mg, 14 mmol) in MeOH (4 ml) at 0° C. The mixture was stirred for 0.5 h at 0° C., filtrated and the filtrate was used directly in next step. The compound 403-38 (500 mg, 0.934 mmol) and NEt₃ (472 mg, 4.67 mmol) was added to the above filtrate. The resulting mixture was stirred at 65° C. overnight. The solution was concentrated and purified by column chromatography on silica gel (CH₂Cl₂/MeOH=150/1) to provide compound 404-38 as a pale white solid (77 mg, 14%): LCMS: 585 [M+1]⁺.

### Step 14f. 6-(2-(6-Amino-8-(6-iodobenzo[d][1,3]dioxol-5-ylthio)-9H-purin-9-yl)ethylamino)-N-hydroxyhexanamide (Compound 38)

The title compound 38 was prepared as a pale white solid (17 mg, 22%) from compound 404-38 (77 mg, 0.13 mmol) and freshly prepared NH₂OH methanol solution (1.77 M, 3 mL) using a procedure similar to that described for compound 11 (Example 6): m.p. 154˜160° C., LCMS: 586 [M+1]; ¹H NMR (DMSO-d₆): δ 1.23 (m, 4H) 1.44 (m, 2H), 1.91 (t, 2H, J=7.4 Hz), 2.45 (t, 2H), 2.81 (t, 2H, J=6.3 Hz), 4.21 (t, 2H, J=6.8 Hz), 6.06 (s, 2H), 6.82 (s, 1H), 7.35 (s, 2H), 7.47 (s, 1H), 8.15 (s, 1H), 8.64 (s, 1H).

### Example 15

### Preparation of 7-(2-(6-amino-8-(6-iodobenzo[d][1,3]dioxol-5-ylthio)-9H-purin-9-yl)ethylamino)-N-hydroxyheptanamide (Compound 39)

### Step 15a. Ethyl 7-(2-(6-amino-8-(6-iodobenzo[d][1,3]dioxol-5-ylthio)-9H-purin-9-yl)ethylamino)heptanoate (Compound 404-39)

The title compound 404-39 was prepared as a pale white solid (100 mg, 17%) from compound 403-14 (500 mg, 0.93 mmol), ethyl 7-aminoheptanoate hydrochloride (2936 mg, 14 mmol) and KOH (785 mg, 14 mmol) in MeOH (8.0 mL) using a procedure similar to that described for compound 404-38 (Example 14): LCMS: 613 [M+1]⁺.

### Step 15b. 7-(2-(6-Amino-8-(6-iodobenzo[d][1,3]dioxol-5-ylthio)-9H-purin-9-yl)ethylamino)-N-hydroxyheptanamide (Compound 39)

The title compound 39 was prepared as a pale white solid (30 mg, 31%) from compound 404-39 (100 mg, 0.16 mmol) and freshly prepared NH₂OH methanol solution (1.77 M, 3 mL) using a procedure similar to that described for compound 11 (Example 6): m.p. 106˜115° C. LCMS: 600 [M+1]; ¹H NMR (DMSO-d₆): δ 1.26 (m, 6H) 1.47 (m, 2H), 1.69 (s, 1H), 1.91 (t, 2H, J=7.4 Hz), 2.44 (t, 2H), 2.81 (t, 2H, J=6.6 Hz), 4.21 (t, 2H, J=6.3 Hz), 6.06 (s, 2H), 6.82 (s, 1H), 7.36 (s, 2H), 7.47 (s, 1H), 8.15 (s, 1H), 8.65 (s, 1H), 10.30 (s, 1H).

### Example 16

### Preparation of 8-(2-(6-amino-8-(6-bromobenzo[d][1,3]dioxol-5-ylthio)-9H-purin-9-yl)ethylamino)-N-hydroxyoctanamide (Compound 41)

### Step 16a. Methyl 8-(2-(6-amino-8-(6-bromobenzo[d][1,3]dioxol-5-ylthio)-9H-purin-9-yl)ethylamino)octanoate (Compound 404-41)

The title compound 404-41 was prepared as a viscous pale yellow solid (210 mg, 44%) from compound 403-20 (410 mg, 0.84 mmol), Methyl 8-aminooctanoate hydrochloride (760 mg, 3.63 mmol) and KOH (203 mg, 3.63 mmol) in MeOH (6.0 mL) using a procedure similar to that described for compound 404-20 (Example 11): LC-MS: 566.8 [M+1]⁺.

### Step 16b. 8-(2-(6-Amino-8-(6-bromobenzo[d][1,3]dioxol-5-ylthio)-9H-purin-9-yl)ethylamino)-N-hydroxyoctanamide (Compound 41)

The title compound 41 was prepared as a pale white solid (50 mg, 24%) from compound 404-41 (210 mg, 0.37 mmol) and freshly prepared NH₂OH methanol solution (1.77 M, 3.5 mL) using a procedure similar to that described for compound 11 (Example 6): m.p. 173˜175° C., LC-MS: 567.8 [M+1]⁺; ¹H NMR (300 MHz, DMSO-d₆): δ 1.18 (m, 6H), 1.26 (m, 2H), 1.45 (m, 2H), 1.69 (s, 1H), 1.91 (t, 2H, J=7.2 Hz), 2.44 (t, 2H, J=6.3 Hz), 2.82 (t, 2H, J=6.3 Hz), 4.22 (t, 2H, J=6.3 Hz), 6.08 (s, 2H), 6.83 (s, 1H), 7.34 (s, 1H), 7.35 (s, 2H), 8.15 (s, 1H), 8.64 (s, 1H), 10.30 (s, 1H).

### Example 17

### Preparation of 4-(2-(6-amino-8-(6-bromobenzo[d][1,3]dioxol-5-ylthio)-9H-purin-9-yl)ethoxy)-N-hydroxybutanamide (Compound 27)

### Step 17a. Ethyl 4-(2-(6-amino-8-(6-bromobenzo[d][1,3]dioxol-5-ylthio)-9H-purin-9-yl)ethoxy)butanoate (Compound 501-27)

To a solution of compound 402-20 (82 mg, 0.2 mmol) in DMSO (1.2 mL) was added KOH (13 mg, 0.22 mmol). The mixture was stirred for 1 hour at room temperature and then ethyl 4-bromobutanoate (39 mg, 0.2 mmol) and Bu₄NI (3 mg) was added. The mixture was heated to 55° C. and stirred overnight. The solution was cooled to room temperature and diluted with CH₂Cl₂ (10 mL), washed with H₂O (3 mL×5). The organic layer was separated and dried over Na₂SO₄, filtered, and concentrated to leave a residue which was purified by column chromatography on silica gel (CH₂Cl₂/MeOH=180/1 with 0.5% Et₃N) to provide 501-27 (64 mg, 61%) as a pale yellow solid. LC-MS: 525.7 [M+1]⁺. ¹H NMR (300 MHz, DMSO-d₆): δ 1.14 (t, 3H, J=7.2 Hz), 1.81 (m, 2H), 2.35 (t, 2H, J=7.2 Hz), 3.29 (m, 2H), 4.00 (m, 6H), 6.08 (s, 2H), 6.61 (s, 2H), 7.16 (s, 1H), 7.19 (s, 1H), 8.01 (s, 1H).

### Step 17b. 4-(2-(6-Amino-8-(6-bromobenzo[d][1,3]dioxol-5-ylthio)-9H-purin-9-yl)ethoxy)-N-hydroxybutanamide (Compound 27)

The title compound 37 was prepared as a white solid (34 mg, 35%) from compound 501-27 (98 mg, 0.19 mmol) and freshly prepared NH₂OH methanol solution (1.77 M, 4 mL) using a procedure similar to that described for compound 11 (Example 6): m.p. 209˜211° C., LC-MS: 512.8 [M+1]⁺; ¹H NMR (300 MHz, DMSO-d₆): δ 1.77 (m, 2H), 2.06 (t, 2H, J=7.2 Hz), 3.29 (m, 2H), 3.89 (t, 2H, J=7.2 Hz), 3.98 (t, 2H, J=6.9 Hz), 6.08 (s, 2H), 6.77 (s, 2H), 7.16 (s, 1H), 7.20 (s, 1H), 8.01 (s, 1H), 8.78 (s, 1H), 10.46 (s, 1H).

### Example 18

### Preparation of 5-(2-(6-amino-8-(6-bromobenzo[d][1,3]dioxol-5-ylthio)-9H-purin-9-yl)ethoxy)-N-hydroxypentanamide (Compound 28)

### Step 18a. Methyl 5-(2-(6-amino-8-(6-bromobenzo[d][1,3]dioxol-5-ylthio)-9H-purin-9-yl)ethoxy)pentanoate (Compound 501-28)

The title compound 501-28 was prepared as a pale yellow solid (180 mg, 56%) from compound 402-20 (250 mg, 0.61 mmol), KOH (38 mg, 0.67 mmol), Methyl 5-bromopentanoate (119 mg, 0.61 mmol) and Bu₄NI (10 mg) using a procedure similar to that described for compound 27 (Example 17): LC-MS: 525.8 [M+1]⁺.

### Step 18b. 5-(2-(6-Amino-8-(6-bromobenzo[d][1,3]dioxol-5-ylthio)-9H-purin-9-yl)ethoxy)-N-hydroxypentanamide (Compound 28)

The title compound 28 was prepared as a white solid (120 mg, 66%) from compound 501-28 (180 mg, 0.19 mmol) and freshly prepared NH₂OH methanol solution (1.77 M, 6 mL) using a procedure similar to that described for compound 11 (Example 6): m.p. 181˜183° C., LC-MS: 526.8 [M+1]⁺; ¹H NMR (300 MHz, DMSO-d₆): δ 1.49 (m, 4H), 1.94 (t, 2H, J=6.9 Hz), 3.29 (m, 2H), 3.96 (m, 4H), 6.08 (s, 2H), 6.59 (s, 2H), 7.17 (s, 1H), 7.19 (s, 1H), 8.01 (s, 1H), 8.67 (s, 1H), 10.32 (s, 1H).

### Example 19

### Preparation of 6-(2-(6-amino-8-(6-bromobenzo[d][1,3]dioxol-5-ylthio)-9H-purin-9-yl)ethoxy)-N-hydroxyhexanamide (compound 29)

### Step 19a. Ethyl 6-(2-(6-amino-8-(6-bromobenzo[d][1,3]dioxol-5-ylthio)-9H-purin-9-yl)ethoxy)hexanoate (Compound 501-29)

The title compound 501-29 was prepared as a pale white solid (200 mg, 59%) from compound 402-20 (250 mg, 0.61 mmol), KOH (38 mg, 0.67 mmol), Ethyl 6-bromohexanoate (136 mg, 0.61 mmol) and Bu₄NI (10 mg) using a procedure similar to that described for compound 27 (Example 17): LCMS: 552 [M+1]⁺.

### Step 19b. 6-(2-(6-Amino-8-(6-bromobenzo[d][1,3]dioxol-5-ylthio)-9H-purin-9-yl)ethoxy)-N-hydroxyhexanamide (Compound 29)

The title compound 29 was prepared as a pale white solid (45 mg, 23%) from compound 501-29 (200 mg, 0.36 mmol) and freshly prepared NH₂OH methanol solution (1.77 M, 5 mL) using a procedure similar to that described for compound 11 (Example 6): m.p. 168˜177° C., LCMS: 539 [M+1]; ¹H NMR (DMSO-d₆): δ 1.21 (m, 2H) 1.50 (m, 4H), 1.90 (t, 2H, J=7.3 Hz), 3.96 (m, 4H), 6.08 (s, 2H), 6.57 (s, 2H), 7.17 (s, 1H), 7.21 (s, 1H), 8.01 (s, 1H), 8.64 (s, 1H), 10.31 (s, 1H).

### Example 20

### Preparation of 7-(2-(6-amino-8-(6-bromobenzo[d][1,3]dioxol-5-ylthio)-9H-purin-9-yl)ethoxy)-N-hydroxyheptanamide (Compound 30)

### Step 20a. Ethyl 7-(2-(6-amino-8-(6-bromobenzo[d][1,3]dioxol-5-ylthio)-9H-purin-9-yl)ethoxy)heptanoate (Compound 501-30)

The title compound 501-30 was prepared as a pale white solid (200 mg, 58%) from compound 402-20 (250 mg, 0.61 mmol), KOH (38 mg, 0.67 mmol), ethyl 7-bromoheptanoate (145 mg, 0.61 mmol) and Bu₄NI (10 mg, 0.027 mmol) using a procedure similar to that described for compound 27 (Example 17): LCMS: 566 [M+1]⁺.

### Step 20b. 7-(2-(6-Amino-8-(6-bromobenzo[d][1,3]dioxol-5-ylthio)-9H-purin-9-yl)ethoxy)-N-hydroxyheptanamide (Compound 30)

The title compound 30 was prepared as a pale white solid (45 mg, 23%) from compound 501-30 (200 mg, 0.35 mmol) and freshly prepared NH₂OH methanol solution (1.77 M, 5 mL) using a procedure similar to that described for compound 11 (Example 6): m.p. 107˜111° C., LCMS: 553 [M+1]⁺; ¹H NMR (DMSO-d₆): δ 1.22 (m, 4H) 1.46 (m, 4H), 1.90 (t, 2H, J=7.4 Hz), 3.92 (m, 4H), 6.08 (s, 2H), 6.57 (s, 2H), 7.16 (s, 1H), 7.20 (s, 1H), 8.01 (s, 1H); ¹H NMR (DMSO-d₆+D₂O): δ 1.20 (m, 4H) 1.45 (m, 4H), 1.88 (t, 2H, J=7.4 Hz), 3.30 (t, 2H) 3.92 (m, 4H), 6.06 (s, 2H), 7.13 (s, 1H), 7.18 (s, 1H), 8.02 (s, 1H).

### Example 21

### Preparation of 7-(6-amino-8-(6-chlorobenzo[d][1,3]dioxol-5-ylthio)-9H-purin-9-yl)-N-hydroxyheptanamide (Compound 31)

### Step 21a. 8-(6-Chlorobenzo[d][1,3]dioxol-5-ylthio)-9H-purin-6-amine (compound 105-31)

A mixture of compound 104 (0.5 g, 3.64 mmol), 5-chloro-6-iodobenzo[d][1,3]dioxole (1.27 g, 5.47 mmol), neocuproine hydrate (62.3 mg, 0.36 mmol), CuI (57 mg, 0.36 mmol) and NaO-t-Bu (345 mg, 4.37 mmol) in anhydrous DMF (25 mL) was stirred for 24 h at 110° C. (oil bath) under nitrogen atmosphere. The solvent was removed under high vacuum and the crude purified by column chromatography on silica gel (CH₂Cl₂/MeOH=30/1) to provide target compound 105-31 as a yellow solid (281 mg, 24%): LCMS: 322 [M+1]; ¹H NMR (DMSO-d₆): δ 6.12 (s, 2H), 7.05 (s, 1H), 7.22 (s, 2H), 7.27 (s, 1H), 8.07 (s, 1H), 13.23 (s, 1H).

### Step 21b. Ethyl 7-(6-amino-8-(6-chlorobenzo[d][1,3]dioxol-5-ylthio)-9H-purin-9-yl)heptanoate (Compound 106-31)

A mixture of compound 105-31 (403 mg, 1.25 mmol), Cs₂CO₃ (692.2 mg, 2.13 mmol), ethyl 7-bromoheptanoate (446 mg, 1.88 mol) and anhydrous DMF (25 mL) was stirred for 6 h at 85° C. The solvent was removed under high vacuum and the crude purified by column chromatography on silica gel (CH₂Cl₂/MeOH=100/1) to provide target compound 106-31 as a yellow viscous solid (230 mg, 38.5%): LCMS: 478 [M+1]⁺. ¹H NMR (DMSO-d₆): δ 1.16 (m, 7H), 1.44 (m, 2H), 1.65 (m, 2H), 2.24 (t, 2H, J=7.2 Hz), 4.02 (q, 2H, J₁=6.9 Hz, J₂=14.1 Hz), 4.14 (t, 2H, J=6.9 Hz), 6.11 (s, 2H), 6.88 (s, 1H), 7.27 (s, 1H), 7.40 (s, 2H), 8.15 (s, 1H).

### Step 21c. 7-(6-Amino-8-(6-chlorobenzo[d][1,3]dioxol-5-ylthio)-9H-purin-9-yl)-N-hydroxyheptanamide (Compound 31)

The title compound 31 was prepared as a pale white solid (75 mg, 55.5%) from compound 106-31 (140 mg, 0.29 mmol) and freshly prepared NH₂OH methanol solution (1.77 M, 4 mL) using a procedure similar to that described for compound 11 (Example 6): m.p. 128˜134° C., LCMS: 465 [M+1]⁺; ¹H NMR (DMSO-d₆): δ 1.20 (m, 4H), 1.40 (m, 2H), 1.65 (m, 2H), 1.90 (t, 2H, J=7.5 Hz), 4.13 (t, 2H, J=6.9 Hz), 6.11 (s, 2H), 6.89 (s, 1H), 7.28 (s, 1H), 7.40 (s, 2H), 8.15 (s, 1H), 8.66 (s, 1H), 10.33 (s, 1H).

### Example 22

### Preparation of 7-(6-amino-8-(6-iodobenzo[d][1,3]dioxol-5-ylthio)-9H-purin-9-yl)-N-hydroxyheptanamide (Compound 32)

### Step 22a. 8-(6-Iodobenzo[d][1,3]dioxol-5-ylthio)-9H-purin-6-amine (compound 105-32)

A mixture of compound 104 (0.8 g, 4.78 mmol), 5,6-diiodobenzo[d][1,3]dioxole (2.68 g, 7.18 mmol), neocuproine hydrate (100 mg, 0.48 mmol), CuI (91.1 mg, 0.48 mmol) and NaO-t-Bu (0.55 g, 5.74 mmol) in anhydrous DMF (40 mL) was stirred for 24 h at 110° C. (oil bath) under nitrogen atmosphere. The solvent was removed under high vacuum and the crude purified by column chromatography on silica gel (CH₂Cl₂/MeOH=30/1) to provide target compound 105-32 as a yellow solid (348 mg, 17.6%): LCMS: 414 [M+1]; ¹H NMR (DMSO-d₆): δ 6.09 (s, 2H), 7.01 (s, 1H), 7.22 (s, 2H), 7.51 (s, 1H), 8.08 (s, 1H), 13.20 (s, 1H).

### Step 22b. Ethyl 7-(6-amino-8-(6-iodobenzo[d][1,3]dioxol-5-ylthio)-9H-purin-9-yl)heptanoate (Compound 106-32)

The title compound 106-32 was prepared as a yellow viscous solid (250 mg, 53.5%) from compound 105-32 (300 mg, 0.82 mmol), Cs₂CO₃ (454.7 mg, 1.40 mmol), ethyl 7-bromoheptanoate (292.7 mg, 1.23 mol) and anhydrous DMF (15 mL) using a procedure similar to that described for compound 106-31 (Example 21): LCMS: 570 [M+1]⁺. ¹H NMR (DMSO-d₆): δ 1.20 (m, 7H), 1.44 (m, 2H), 1.65 (m, 2H), 2.23 (t, 2H, J=7.2 Hz), 4.02 (q, 2H, J₁=6.9 Hz, J₂=14.1 Hz), 4.13 (t, 2H, J=6.9 Hz), 6.08 (s, 2H), 6.82 (s, 1H), 7.44 (s, 2H), 7.50 (s, 1H), 8.16 (s, 1H).

### Step 22c. 7-(6-Amino-8-(6-iodobenzo[d][1,3]dioxol-5-ylthio)-9H-purin-9-yl)-N-hydroxyheptanamide (Compound 32)

The title compound 32 was prepared as a pale white solid (135 mg, 36.8%) from compound 106-32 (244 mg, 0.43 mmol) and freshly prepared NH₂OH methanol solution (1.77 M, 6 mL) using a procedure similar to that described for compound 11 (Example 6): m.p. 200˜203° C., LCMS: 557 [M+1]⁺; ¹H NMR (DMSO-d₆): δ 1.20 (m, 4H), 1.43 (m, 2H), 1.62 (m, 2H), 1.90 (t, 2H, J=7.5 Hz), 4.11 (t, 2H, J=6.9 Hz), 6.07 (s, 2H), 7.00 (s, 1H), 6.82 (s, 1H), 7.42 (s, 2H), 7.50 (s, 1H), 8.15 (s, 1H), 8.66 (s, 1H), 10.32 (s, 1H).

### Example 23

### Preparation of 7-(4-amino-2-(6-chlorobenzo[d][1,3]dioxol-5-ylthio)-1H-imidazo[4,5-c]pyridin-1-yl)-N-hydroxyheptanamide (Compound 34)

### Step 23a. 2-Chloro-N-(4-methoxybenzyl)-3-nitropyridin-4-amine (Compound 602)

To a stirred solution of compound 601 (1 g, 5.18 mmol) in DMF (8.6 mL) was added (4-methoxyphenyl)methanamine (0.71 g, 5.18 mmol) and triethylamine (0.644 mL). The reaction mixture was stirred at room temperature for 2 h. The mixture was evaporated to remove DMF and purified by column chromatography on silica gel (EtOAc/petroleum at 10:1) to obtain 602 as a yellow solid (1.32 g, 87%): LCMS: 294 [M+1]⁻; ¹H NMR (DMSO-d₆): δ 3.72 (s, 3H), 4.40 (d, 2H, J=6.3 Hz), 6.81 (d, 1H, J=5.7 Hz), 6.91 (d, 2H, J=9.0 Hz), 7.25 (d, 2H, J=8.4 Hz), 7.95 (d, 1H, J=5.4 Hz), 8.02 (t, 1H, J=5.7 Hz).

### Step 23b. 2-Chloro-N-(4-methoxybenzyl)pyridine-3,4-diamine (603)

To a stirred solution of compound 602 (1.32 g, 4.49 mmol) in methanol (66 mL) was added water (6.6 mL), iron powder (2.51 g, 44.9 mmol) and concentrated HCl solution (1 mL). The reaction mixture was stirred at room temperature for 30 min, and then heated to reflux overnight. The mixture was adjusted to pH 11 with 6N NaOH. The resulting solid was filtered and washed with methanol (10 mL). The combined filtrate was concentrated to leave a residue which was purified by column chromatography on silica gel (EtOAc/petroleum at 2:1) to obtain 603 as a light green solid (712 mg, 60%): LCMS: 264 [M+1]⁺; ¹H NMR (DMSO-d₆): δ 3.73 (s, 3H), 4.31 (d, 2H, J=5.7 Hz), 4.81 (s, 2H), 6.33 (m, 2H), 6.90 (d, 2H, J=8.7 Hz), 7.26 (d, 2H, J=9.0 Hz), 7.34 (d, 1H, J=5.1 Hz).

### Step 23c. 4-Chloro-1-(4-methoxybenzyl)-1H-imidazo[4,5-c]pyridine-2(3H)-thione (Compound 604)

A mixture of compound 603 (2 g, 7.6 mmol), carbon disulfide (2.88 g, 37.9 mmol), potassium hydroxide (2.12 g, 37.9 mmol) in ethanol (11.5 mL) and water (1.5 mL) was heated at reflux overnight. The reaction was cooled down to room temperature and 100 mL of water was added. The mixture was adjusted to pH 7 with acetic acid and then extracted with two portions of methylene chloride. The extract was concentrated at reduced pressure and purified by column chromatography on silica gel (EtOAc/petroleum at 5:1) to obtain compound 604 as a white solid (2 g, 86%): LCMS: 306 [M]⁻; ¹H NMR (DMSO-d₆): δ 3.68 (s, 3H), 6.41 (s, 2H), 6.86 (d, 2H, J=8.7 Hz), 7.36 (m, 3H), 8.07 (d, 1H, J=5.4 Hz), 13.74 (s, 1H).

### Step 23d. 4-Amino-1-(4-methoxybenzyl)-1H-imidazo[4,5-c]pyridine-2(3H)-thione (Compound 605)

A mixture of compound 604 (1 g, 3.25 mmol) and sodium amide (3 g, 77 mmol) in 25 mL liquid ammonia was charged in a no air sealed tube, and stirred at room temperature for 30 h. The mixture was cooled to −40° C. and the tube was opened. Ethanol was added carefully to the reaction until no gas generated. 200 mL of water was added and adjusted the mixture to pH 7 with acetic acid. The resulting mixture was filtered to obtain crude which was purified by column chromatography on silica gel (methylene chloride/methanol at 50:1) to obtain compound 605 as a white solid (718 mg, 77%): LCMS: 287 [M]⁺; ¹H NMR (DMSO-d₆): δ 3.68 (s, 3H), 5.31 (s, 2H), 6.06 (s, 2H), 6.59 (d, 1H, J=6.3 Hz), 6.85 (d, 2H, J=9.0 Hz), 7.33 (d, 2H, J=8.4 Hz), 7.64 (d, 1H, J=5.7 Hz), 12.53 (s, 1H).

### Step 23e. 2-(6-Chlorobenzo[d][1,3]dioxol-5-ylthio)-1-(4-methoxybenzyl)-1H-imidazo[4,5-c]pyridin-4-amine (Compound 606-34)

A mixture of compound 605 (543 mg, 1.9 mmol), 5-chloro-6-iodobenzo[d][1,3]dioxole (1.07 g, 3.79 mmol), neocuproine hydrate (40 mg, 0.19 mmol), CuI (36 mg, 0.19 mmol) and NaOt-Bu (273 mg, 2.84 mmol) in anhydrous DMF (24 mL) was stirred for 24 h at 110° C. (oil bath) under nitrogen atmosphere. The solvent was removed under high vacuum and the crude purified by column chromatography on silica gel (CH₂Cl₂/MeOH at 100/1) to obtain target compound 606-34 as a brown solid (506 mg, 61%): LCMS: 441 [M+1]⁺; ¹H NMR (DMSO-d₆): δ 3.69 (s, 3H), 5.37 (s, 2H), 6.04 (s, 2H), 6.41 (s, 2H), 6.55 (s, 1H), 6.80 (d, 2H, J=8.7 Hz), 7.04 (d, 2H, J=9.3 Hz), 7.17 (s, 1H), 7.73 (s, 1H).

### Step 23f. 2-(6-Chlorobenzo[d][1,3]dioxol-5-ylthio)-1H-imidazo[4,5-c]pyridin-4-amine (607-34)

Compound 606-34 (506 mg, 1.14 mmol) was dissolved in TFA (4 mL) and stirred for 2 h at 80° C. The solution was evaporated and the residual was adjust to pH 7 with saturated NaHCO₃ and filtered. The precipitate was purified by column chromatography on silica gel (CH₂Cl₂/MeOH at 30/1) to obtain target compound 607-34 as a yellow solid (300 mg, 82%): LCMS: 321 [M+1]⁺; ¹H NMR (DMSO-d₆): δ 6.11 (s, 2H), 6.56 (m, 3H), 7.04 (s, 1H), 7.26 (s, 1H), 7.49 (s, 2H), 12.25 (s, 1H).

### Step 23g. Ethyl 7-(4-amino-2-(6-chlorobenzo[d][1,3]dioxol-5-ylthio)-1H-imidazo[4,5-c]pyridin-1-yl)heptanoate (Compound 608-34)

A mixture of compound 607-34 (300 mg, 0.935 mmol), ethyl 7-bromoheptanoate (333 mg, 1.403 mmol), Cs₂CO₃ (517 mg, 1.59 mmol) in DMF (12 mL) was stirred at 85° C. for 2 h. DMF was evaporated under vacuum, and the residue was purified by column chromatography on silica gel (methylene chloride/methanol at 100:1) to yield compound 608-34 as a white solid (300 mg, 67%): LCMS: 477 [M+1]⁺; ¹H NMR (DMSO-d₆): δ 1.15 (m, 7H), 1.42 (m, 2H), 1.58 (m, 2H), 2.21 (t, 2H, J=7.2 Hz), 4.02 (q, 2H, J=7.5 Hz), 4.16 (t, 2H, J=7.2 Hz), 6.08 (s, 2H), 6.37 (s, 2H), 6.73 (s, 1H), 6.80 (d, 1H, J=5.1 Hz), 7.25 (s, 1H), 7.70 (d, 1H, J=6.0 Hz).

### Step 23h. 7-(4-Amino-2-(6-chlorobenzo[d][1,3]dioxol-5-ylthio)-1H-imidazo[4,5-c]pyridin-1-yl)-N-hydroxyheptanamide (Compound 34)

The title compound 34 was prepared as a white solid (98 mg, 34%) from compound 608-34 (300 mg, 0.63 mmol) and freshly prepared NH₂OH methanol solution (1.77 M, 3 mL) using a procedure similar to that described for compound 11 (Example 6): m.p. 144˜148° C., LCMS: 464 [M+1]⁺; ¹H NMR (DMSO-d₆): δ 1.22 (m, 4H), 1.42 (m, 2H), 1.65 (m, 2H), 1.90 (t, 2H, J=7.2 Hz), 4.29 (t, 2H, J=6.9 Hz), 6.14 (s, 2H), 7.07 (s, 1H), 7.31 (m, 2H), 7.73 (d, 1H, J=6.9 Hz), 8.51 (s, 2H), 10.32 (s, 1H), 13.04 (s, 1H).

### Example 24

### Preparation of 7-(4-amino-2-(6-bromobenzo[d][1,3]dioxol-5-ylthio)-1H-imidazo[4,5-c]pyridin-1-yl)-N-hydroxyheptanamide (Compound 35)

### Step 24a. 2-(6-Bromobenzo[d][1,3]dioxol-5-ylthio)-1-(4-methoxybenzyl)-1H-imidazo[4,5-c]pyridin-4-amine (606-35)

The title compound 606-35 was prepared as a brown solid (584 mg, 49%) from compound 605 (700 mg, 2.44 mmol), 5-bromo-6-iodobenzo[d][1,3]dioxole (1.20 g, 3.66 mmol), neocuproine hydrate (51 mg, 0.244 mmol), CuI (46 mg, 0.244 mmol) and NaOt-Bu (234 mg, 2.44 mmol) in anhydrous DMF (31 mL) using a procedure similar to that described for compound 606-34 (Example 23): LCMS: 485 [M+1]⁺; ¹H NMR (DMSO-d₆): δ 3.29 (s, 3H), 5.39 (s, 2H), 6.04 (s, 2H), 6.54 (s, 1H), 6.81 (m, 3H), 6.91 (d, 1H, J=5.4 Hz), 7.06 (d, 2H, J=8.6 Hz), 7.29 (s, 1H), 7.71 (d, 1H, J=6.0 Hz).

### Step 24b. 2-(6-Bromobenzo[d][1,3]dioxol-5-ylthio)-1H-imidazo[4,5-c]pyridin-4-amine (Compound 607-35)

Compound 606-35 (557 mg, 1.15 mmol) was dissolved in TFA (4 mL) and stirred for 2 h at 80° C. The solution was evaporated and the residual was adjusted to pH 7 with saturated NaHCO₃ and filter. The precipitate was purified by column chromatography on silica gel (CH₂Cl₂/MeOH at 30/1) to obtain target compound 607-35 as a white solid (308 mg, 74%): LCMS: 365 [M+1]⁺; ¹H NMR (DMSO-d₆): δ 6.07 (s, 2H), 6.58 (s, 2H), 6.69 (d, 1H, J=6.0 Hz), 6.98 (s, 1H), 7.34 (s, 1H), 7.47 (d, 1H, J=5.7 Hz).

### Step 24c. Ethyl 7-(4-amino-2-(6-bromobenzo[d][1,3]dioxol-5-ylthio)-1H-imidazo[4,5-c]pyridin-1-yl)heptanoate (Compound 608-35)

The title compound 608-35 was prepared as a white solid (240 mg, 56%) from compound 607-35 (302 mg, 0.827 mmol), ethyl 7-bromoheptanoate (294 mg, 1.24 mmol), Cs₂CO₃ (457 mg, 1.406 mmol) in DMF (12 mL) using a procedure similar to that described for compound 608-34 (Example 23): LCMS: 521 [M+1]⁺; ¹H NMR (DMSO-d₆): δ 1.16 (m, 7H), 1.41 (m, 2H), 1.58 (m, 2H), 2.21 (t, 2H, J=7.5 Hz), 4.02 (q, 2H, J=6.9 Hz), 4.16 (t, 2H, J=6.9 Hz), 6.07 (s, 2H), 6.40 (s, 2H), 6.67 (s, 1H), 6.80 (d, 1H, J=5.7 Hz), 7.36 (s, 1H), 7.71 (d, 1H, J=5.7 Hz).

### Step 24d. 7-(4-Amino-2-(6-bromobenzo[d][1,3]dioxol-5-ylthio)-1H-imidazo[4,5-c]pyridin-1-yl)-N-hydroxyheptanamide (Compound 35)

The title compound 35 was prepared as a white solid (182 mg, 79%) from compound 608-35 (236 mg, 0.453 mmol) and freshly prepared NH₂OH methanol solution (1.77 M, 3 mL) using a procedure similar to that described for compound 11 (Example 6): m.p. 179˜181° C., LCMS: 508 [M+1]⁺; ¹H NMR (DMSO-d₆): δ 1.17 (m, 4H), 1.36 (m, 2H), 1.57 (m, 2H), 1.88 (t, 2H, J=6.9 Hz), 4.15 (t, 2H, J=7.2 Hz), 6.08 (s, 2H), 6.43 (s, 2H), 6.67 (s, 1H), 6.81 (d, 1H, J=5.4 Hz), 7.36 (s, 1H), 7.71 (d, 1H, J=5.7 Hz), 8.66 (s, 1H), 10.32 (s, 1H).

### Example 25

### Preparation of 7-(4-amino-2-(6-iodobenzo[d][1,3]dioxol-5-ylthio)-1H-imidazo[4,5-c]pyridin-1-yl)-N-hydroxyheptanamide (Compound 36)

### Step 25a. 2-(6-Iodobenzo[d][1,3]dioxol-5-ylthio)-1-(4-methoxybenzyl)-1H-imidazo[4,5-c]pyridin-4-amine (Compound 606-36)

The title compound 606-36 was prepared as a brown solid (734 mg, 55%) from compound 605 (725 mg, 2.53 mmol), 5,6-diiodobenzo[d][1,3]dioxole (1.89 g, 5.06 mmol), neocuproine hydrate (53 mg, 0.253 mmol), CuI (48 mg, 0.253 mmol) and NaOt-Bu (365 mg, 3.80 mmol) in anhydrous DMF (32 mL) using a procedure similar to that described for compound 606-34 (Example 23): LCMS: 533 [M+1]⁺; ¹H NMR (DMSO-d₆): δ 3.69 (s, 3H), 5.35 (s, 2H), 6.01 (s, 2H), 6.47 (s, 1H), 6.80 (d, 2H, J=9.0 Hz), 7.06 (d, 2H, J=8.6 Hz), 7.41 (s, 1H).

### Step 25b. 2-(6-Iodobenzo[d][1,3]dioxol-5-ylthio)-1H-imidazo[4,5-c]pyridin-4-amine (Compound 607-36)

Compound 606-36 (730 mg, 1.37 mmol) was dissolved in TFA (4.8 mL) and stirred for 2 h at 80° C. The solution was evaporated and the residual was adjusted to pH 7 with saturated NaHCO₃ and filter. The precipitate was purified by column chromatography on silica gel (CH₂Cl₂/MeOH at 30/1) to obtain target compound 607-36 as a yellow solid (526 mg, 93%): LCMS: 413 [M+1]⁺; ¹H NMR (DMSO-d₆): δ 6.09 (s, 2H), 6.73 (m, 3H), 7.03 (s, 1H), 7.52 (m, 2H), 12.45 (s, 1H).

### Step 25c. Ethyl 7-(4-amino-2-(6-iodobenzo[d][1,3]dioxol-5-ylthio)-1H-imidazo[4,5-c]pyridin-1-yl)heptanoate (Compound 608-36)

The title compound 608-36 was prepared as a white solid (149 mg, 61%) from compound 607-36 (178 mg, 0.432 mmol), ethyl 7-bromoheptanoate (154 mg, 0.648 mmol), Cs₂CO₃ (239 mg, 0.734 mmol) in DMF (6.3 mL) using a procedure similar to that described for compound 608-34 (Example 23): LCMS: 569 [M+1]⁺; ¹H NMR (DMSO-d₆): δ 1.16 (m, 7H), 1.42 (m, 2H), 1.57 (m, 2H), 2.22 (t, 2H, J=7.2 Hz), 4.03 (q, 2H, J=7.5 Hz), 4.15 (t, 2H, J=7.2 Hz), 6.04 (s, 2H), 6.39 (s, 2H), 6.65 (s, 1H), 6.80 (d, 1H, J=6.0 Hz), 7.48 (s, 1H), 7.71 (d, 1H, J=5.7 Hz).

### Step 25d. 7-(4-Amino-2-(6-iodobenzo[d][1,3]dioxol-5-ylthio)-1H-imidazo[4,5-c]pyridin-1-yl)-N-hydroxyheptanamide (Compound 36)

The title compound 36 was prepared as a white solid (45 mg, 33%) from compound 608-36 (140 mg, 0.246 mmol) and freshly prepared NH₂OH methanol solution (1.77 M, 3 mL) using a procedure similar to that described for compound 11 (Example 6): m.p. 191˜193° C., LCMS: 556 [M+1]⁺; ¹H NMR (DMSO-d₆): δ 1.18 (m, 4H), 1.37 (m, 2H), 1.57 (m, 2H), 1.89 (t, 2H, J=6.9 Hz), 4.14 (t, 2H, J=7.2 Hz), 6.04 (s, 2H), 6.42 (s, 2H), 6.66 (s, 1H), 6.80 (d, 1H, J=5.7 Hz), 7.49 (s, 1H), 7.71 (d, 1H, J=5.7 Hz), 8.66 (s, 1H), 10.31 (s, 1H).

### Example 26

### Preparation of 6-(4-amino-2-(6-iodobenzo[d][1,3]dioxol-5-ylthio)-1H-imidazo[4,5-c]pyridin-1-yl)-N-hydroxyhexanamide (Compound 42)

### Step 26a. Ethyl 6-(4-amino-2-(6-iodobenzo[d][1,3]dioxol-5-ylthio)-1H-imidazo[4,5-c]pyridin-1-yl)hexanoate (Compound 608-42)

The title compound 608-42 was prepared as a white solid (260 mg, 64%) from compound 607-36 (300 mg, 0.73 mmol), ethyl 6-bromohexanoate (243 mg, 1.09 mmol), Cs₂CO₃ (404 mg, 1.24 mmol) in DMF (4.0 mL) using a procedure similar to that described for compound 608-34 (Example 23): LCMS: 555 [M+1]⁺; ¹H NMR (DMSO-d₆): δ 1.14 (t, 2H, J=7.2 Hz), 1.19 (m, 2H), 1.47 (m, 2H), 1.57 (m, 2H), 2.19 (t, 2H, J=7.2 Hz), 4.01 (q, 2H, J=7.2 Hz), 4.16 (t, 2H, J=6.9 Hz), 6.05 (s, 2H), 6.55 (s, 2H), 6.68 (s, 1H), 6.83 (d, 1H, J=6.0 Hz), 7.48 (s, 1H), 7.70 (d, 1H, J=6.0 Hz).

### Step 26b. 6-(4-Amino-2-(6-iodobenzo[d][1,3]dioxol-5-ylthio)-1H-imidazo[4,5-c]pyridin-1-yl)-N-hydroxyhexanamide (Compound 42)

The title compound 42 was prepared as a white solid (107 mg, 42%) from compound 608-42 (260 mg, 0.47 mmol) and freshly prepared NH₂OH methanol solution (1.77 M, 6 mL) using a procedure similar to that described for compound 11 (Example 6): m.p. 189˜193° C., LCMS: 542 [M+1]⁺; ¹H NMR (DMSO-d₆): δ 1.20 (m, 2H), 1.44 (m, 2H), 1.56 (m, 2H), 1.87 (t, 2H, J=7.2 Hz), 4.13 (t, 2H, J=7.2 Hz), 6.05 (s, 2H), 6.36 (s, 2H), 6.67 (s, 1H), 6.78 (d, 1H, J=6.0 Hz), 7.48 (s, 1H), 7.70 (d, 1H, J=5.7 Hz).

### Biological Assays:

As stated hereinbefore the derivatives defined in the present invention possess anti-proliferation activity. These properties may be assessed, for example, using one or more of the procedures set out below:

(a) An In Vitro Assay which Determines the Ability of a Test Compound to Inhibit Hsp90 Chaperone Activity.

The Hsp90 chaperone assay was performed to measure the ability of HSP90 protein to refold the heat-denatured luciferase protein. HSP90 was first incubated with different concentrations of test compounds in denaturation buffer (25 mM Tris, pH7.5, 8 mM MgSO4, 0.01% bovine gamma globulin and 10% glycerol) at room temperature for 30 min. Luciferase protein was added to denaturation mix and incubated at 50° C. for 8 min. The final concentration of HSP90 and luciferase in denaturation mixture were 0.375 μM and 0.125 μM respectively. A 5 μl sample of the denatured mix was diluted into 25 μl of renaturation buffer (25 mM Tris, pH7.5, 8 mM MgSO4, 0.01% bovine gamma globulin and 10% glycerol, 0.5 mM ATP, 2 mM DTT, 5 mM KCl, 0.3 μM HSP70 and 0.15 μM HSP40). The renaturation reaction was incubated at room temperature for 150 min, followed by dilution of 10 μl of the renatured sample into 90 μl of luciferin reagent (Luclite, PerkinElmer Life Science). The mixture was incubated at dark for 5 min before reading the luminescence signal on a TopCount plate reader (PerkinElmer Life Science).

(b) An In Vitro Assay which Determines the Ability of a Test Compound to Inhibit HDAC Enzymatic Activity.

HDAC inhibitors were screened using an HDAC fluorimetric assay kit (AK-500, Biomol, Plymouth Meeting, Pa.). Test compounds were dissolved in dimethylsulphoxide (DMSO) to give a 20 mM working stock concentration. Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm. Each assay was setup as follows: Defrosted all kit components and kept on ice until use. Diluted HeLa nuclear extract 1:29 in Assay Buffer (50 mM Tris/Cl, pH 8.0, 137 mM NaCl, 2.7 mM KCl, 1 mM MgCl2). Prepared dilutions of Trichostatin A (TSA, positive control) and tested compounds in assay buffer (5× of final concentration). Diluted Fluor de Lys™ Substrate in assay buffer to 100 uM (50 fold=2× final). Diluted Fluor de Lys™ developer concentrate 20-fold (e.g. 50 μl plus 950 μl Assay Buffer) in cold assay buffer. Second, diluted the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Added Assay buffer, diluted trichostatin A or test inhibitor to appropriate wells of the microtiter plate. Added diluted HeLa extract or other HDAC sample to all wells except for negative controls. Allowed diluted Fluor de Lys™ Substrate and the samples in the microtiter plate to equilibrate to assay temperature (e.g. 25 or 37° C. Initiated HDAC reactions by adding diluted substrate (25 μl) to each well and mixing thoroughly. Allowed HDAC reactions to proceed for 1 hour and then stopped them by addition of Fluor de Lys™ Developer (50 μl). Incubated plate at room temperature (25° C.) for 10-15 min. Read samples in a microtiter-plate reading fluorimeter capable of excitation at a wavelength in the range 350-380 nm and detection of emitted light in the range 440-460 nm.

The following TABLE 7-B lists compounds representative of the invention and their activity in HDAC and HSP90 assays. In these assays, the following grading was used: I≧10 μM, 10 μM>II>1 μM, 1 μM>III>0.1 μM, and IV≦0.1 μM for IC₅₀.

TABLE 7-B

| Compound No. | HDAC | HSP90 |
|---|---|---|
| 5 | III | III |
| 6 | IV | III |
| 12 | IV | |
| 13 | I | I |
| 14 | III | |
| 16 | IV | I |
| 18 | IV | I |
| 20 | I | III |
| 23 | IV | III |
| 24 | IV | III |
| 27 | I | I |
| 28 | II | I |
| 29 | II | I |
| 30 | IV | I |
| 31 | IV | II |
| 32 | IV | IV |
| 33 | I | III |
| 34 | IV | II |
| 35 | IV | II |
| 36 | IV | III |
| 37 | I | III |
| 38 | III | III |
| 39 | IV | III |
| 40 | I | I |
| 41 | IV | |

TABLE 8-A
SECTION 8:

| | |
|---|---|
| | (IX) |
| 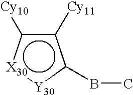 | |

| | |
|---|---|
| | (X) |
| 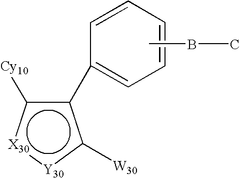 | |

| Compound # | Structure |
|---|---|
| 1 | 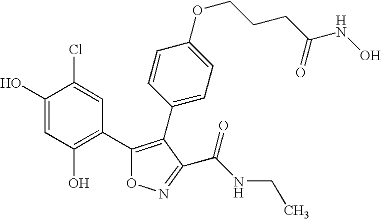 |
| 2 | 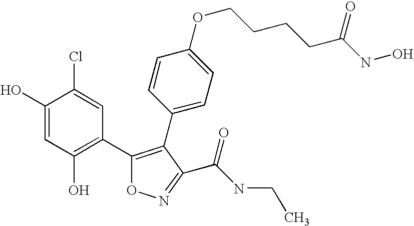 |
| 3 | 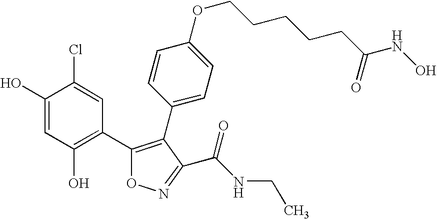 |
| 4 | 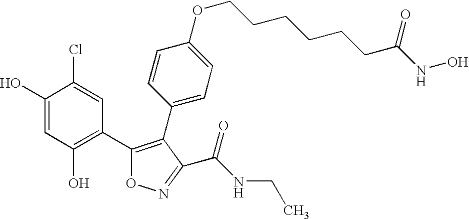 |
| 5 | 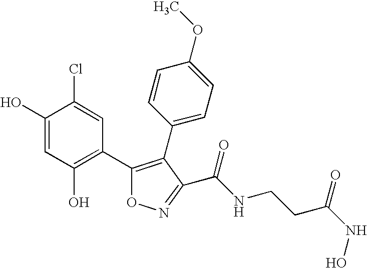 |
| 6 | 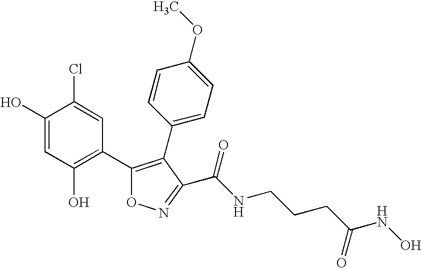 |
| 7 | 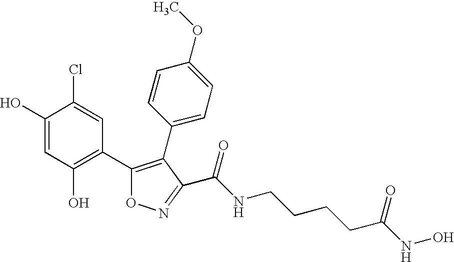 |
| 8 | 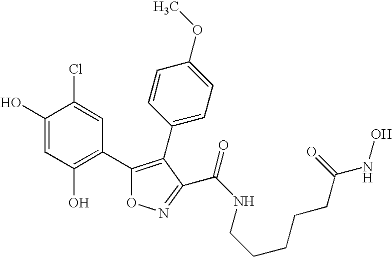 |
| 9 | 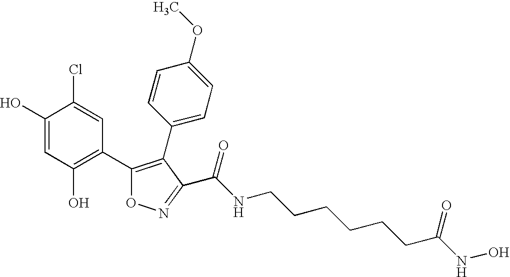 |
| 10 | 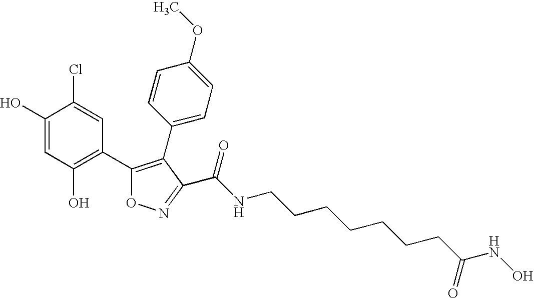 |

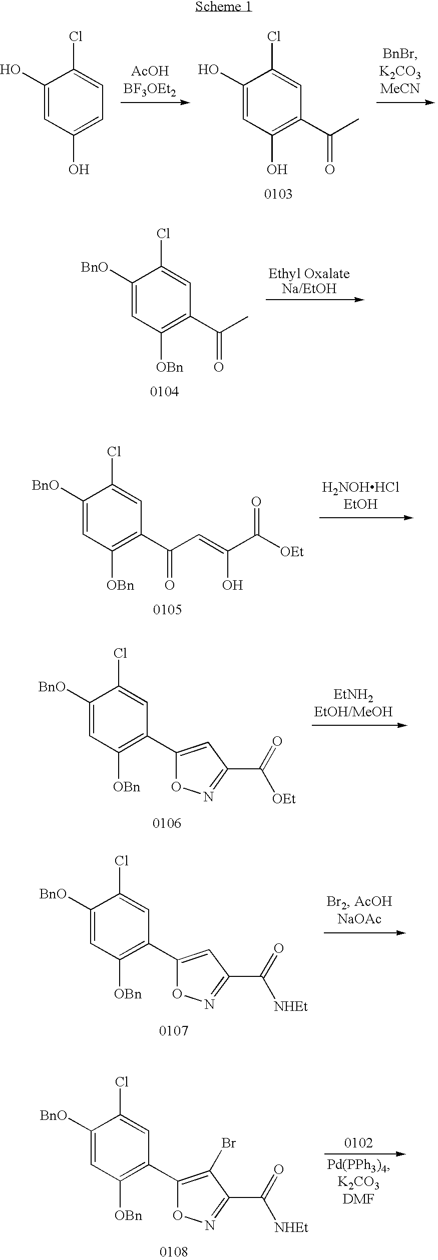

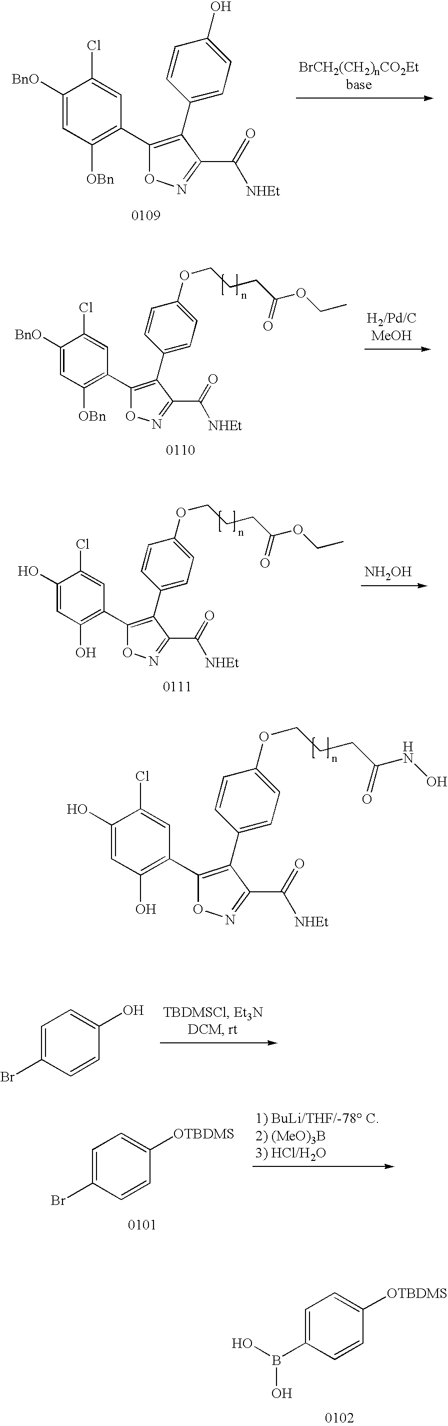

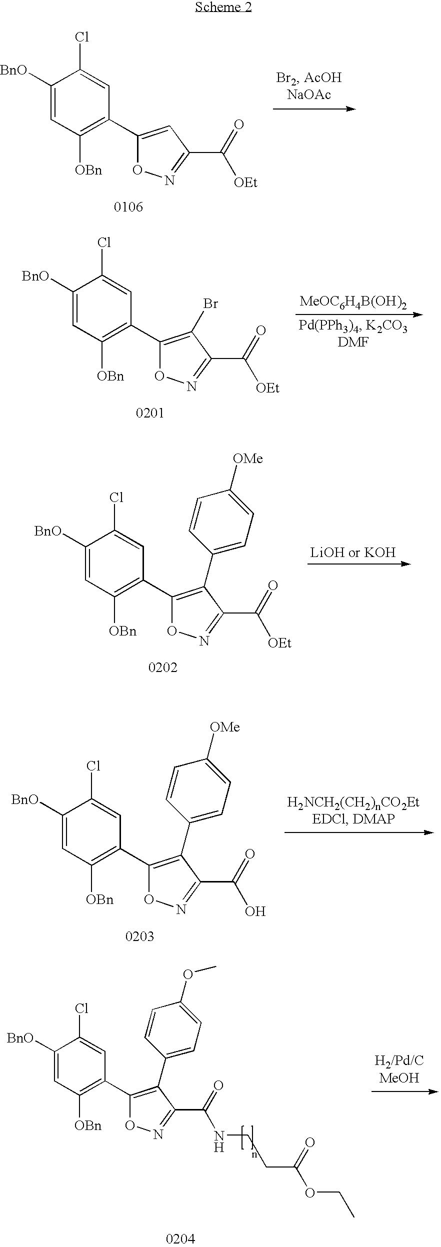

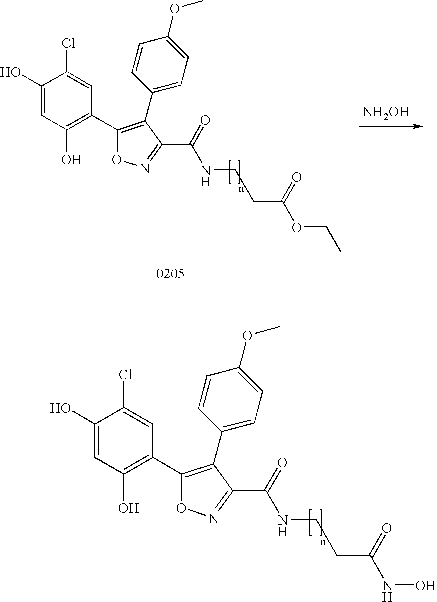

### Example 1

### Preparation of 5-(5-chloro-2,4-dihydroxyphenyl)-N-ethyl-4-(4-(4-(hydroxyamino)-4-oxobutoxy)phenyl)isoxazole-3-carboxamide (Compound 1)

### Step 1a: (4-Bromophenoxy)(tert-butyl)dimethylsilane (compound 0101)

Et₃N (16.7 g, 115.6 mmol) was added dropwise to a solution of compound 4-bromophenol (10.0 g, 57.8 mmol) and TBSCl (11.3 g, 75.14 mmol) in DMC (100 ml) at room temperature and the mixture was stirred for 2 h. After solvent was removed, 200 ml of petroleum ether was added. The organic layer was wash with water and brine, dried over anhydrous Na₂SO₄, filtered through a short silica gel column and evaporated to obtain 0101 as a colorless oil (16.6 g, 100%): ¹H NMR (CDCl₃): δ 0.18 (s, 6H), 2.71 (t, J=6 Hz, 2H), 0.98 (s, 9H), 6.70-6.73 (m, 2H), 7.30-7.33 (m, 2H).

### Step 1b: 4-(Tert-butyldimethylsilyloxy)phenylboronic acid (Compound 0102)

To a solution of compound 0101 (1.548 g, 5.389 mmol) in dry THF (20 ml) was added dropwise a 2.5 M n-BuLi in hexane solution (2.5 ml, 6.326 mmol,) at −78° C. for 15 min under N₂. After the mixture was stirred at −78° C. for 0.5 h, trimethyl borate (730 mg, 7.029 mmol) was added dropwise for 15 min to the mixture. The mixture was stirred at −78° C. for additional 1 h and warmed to room temperature. The reaction mixture was quenched with aqueous hydrochloric acid solution (to pH 5-7). The solvent was removed and the residue was extracted with DCM. The organic layer was washed with brine, dried over anhydrous Na₂SO₄, concentrated to give a residue which was washed by petroleum (2 ml) to afford the product 0102 as a white solid (1.102 g, 81%): LCMS: 253 [M+1]⁺.

### Step 1c: 1-(5-Chloro-2,4-dihydroxyphenyl)ethanone (Compound 0103)

To a suspension of 4-chlororesorcinol (21.25 g, 0.147 mol) in boron trifluoride etherate (100 ml) was added acetic acid (8.75 ml) dropwise under N₂. The reaction mixture was stirred at 80° C. overnight and then allowed to cool to room temperature. The mixture was poured into 350 ml of 10% w/v aqueous sodium acetate solution and stirred vigorously for 2.5 h. A light brown solid was precipitated which was filtered, washed with water and petroleum ether, dried to obtain 0103 as a white brown solid (18.49 g, 67.4%): LCMS: 187 [M+1]⁺.

### Step 1d: 1-(2,4-Bis(benzyloxy)-5-chlorophenyl)ethanone (Compound 0104)

Benzyl chloride (23.72 g, 0.187 mol) was added to a mixture of compound 0103 (17.49 g, 0.094 mol) and potassium carbonate (32.33 g, 0.234 mol) in acetonitrile (320 ml). The mixture was heated to reflux for 48 h and allowed to cool to room temperature. After the mixture was evaporated near dryness, it was filtered and the solids were washed with water to remove K₂CO₃ and dried in vacuo. The solids were washed with petroleum (350 ml) and ethyl acetate (15 ml) to obtain the product 0104 as a brown solid (37 g, 100%): LCMS: 367 [M+1]. ¹H NMR (CDCl₃): δ 2.45 (s, 3H), 5.30 (s, 2H), 5.35 (s, 2H), 7.16 (s, 1H), 7.37-7.54 (m, 10H), 7.70 (s, 1H).

### Step 1e: Ethyl 4-(2,4-bis(benzyloxy)-5-chlorophenyl)-2,4-dioxo-butanoate (Compound 0105)

To the solution of compound 0104 (5.0 g, 13.63 mmol) in anhydrous THF (30 ml) was added 60% NaH (1.64 g, 40.89 mmol) slowly. After the mixture was stirred at room temperature for 30 min, diethyl oxalate (3.98 g, 27.26 mmol) was added and the mixture was stirred at 60° C. for 40 min. Then it was allowed to cool to room temperature and acetic acid (2.7 g, 44.98 mmol) was added. It was evaporated near to dryness and 100 ml ethyl acetate was added, washed with water and brine, dried over anhydrous Na₂SO₄. The organic phase was evaporated and the residue was washed with 10-20 ml of ethanol, filtrated to obtain compound 0105 as a light yellow solid (5.0 g, 79%): LCMS: 467 [M+1]⁺. ¹H NMR (DMSO-d₆): δ 1.16 (t, J=6 Hz, 3H), 4.20 (q, J=6 Hz, 2H), 5.36 (s, 2H), 5.39 (s, 2H), 7.23 (s, 1H), 7.29 (s, 1H), 7.38-7.55 (m, 10H), 7.89 (s, 1H).

### Step 1f: Ethyl 5-(2,4-bis(benzyloxy)-5-chlorophenyl)isoxazole-3-carboxylate (Compound 0106)

Hydroxylamine hydrochloride (0.89 g, 12.8 mmol) was added to a suspension of compound 0105 (5.00 g, 10.7 mmol) in absolute ethanol (100 ml). The reaction mixture was heated at refluxing for 4 hours and was allowed to cool to room temperature. The mixture was filtered and the solid was washed with ethanol and dried in vacuo at 45° C. to obtain compound 0106 as a pale yellow solid (4.8 g, 97%): LCMS: 464 [M+1]. ¹H NMR (CDCl₃): δ 1.40 (t, J=6 Hz, 3H), 4.42 (q, J=6 Hz, 2H), 5.12 (s, 2H), 5.15 (s, 2H), 6.61 (s, 1H), 7.01 (s, 1H), 7.35-7.40 (m, 10H), 8.01 (s, 1H).

### Step 1g: 5-(2,4-Bis(benzyloxy)-5-chlorophenyl)-N-ethylisoxazole-3-carboxamide (Compound 0107)

To a flask containing 0106 (4.40 g, 9.51 mmol) was added a solution of ethylamine in ethanol (2.0 M, 40 ml, 80 mmol). The mixture was heated to 80° C. and stirred for 5 h. The mixture was allowed to cool to ice-bath temperature, filtered and the solid was washed with cold ethanol, dried in vacuo to obtain 0107 as a white solid (4.10 g, 93%): LCMS: 463 [M+1]⁺. ¹H NMR (CDCl₃): δ 1.28 (t, J=6 Hz, 3H), 3.44-3.53 (m, 2H), 5.10 (s, 2H), 5.16 (s, 2H), 6.59 (s, 1H), 6.81 (t, J=6 Hz, 1H), 7.08 (s, 1H), 7.25-7.40 (m, 10H), 7.97 (s, 1H).

### Step 1h: 5-(2,4-Bis(benzyloxy)-5-chlorophenyl)-4-bromo-N-ethyl-isoxazole-3-carboxamide (Compound 0108)

A solution of bromine in acetic acid (0.6 M, 306.0 ml, 183.6 mmol) was added to a stirred suspension of 0107 (8.50 g, 18.36 mmol) and potassium acetate (3.97 g, 40.50 mmol) in acetic acid (127 ml) at room temperature. The mixture was stirred at room temperature for 5 min. And saturated solution of Na₂SO₃ was added to the solution. After the mixture was concentrated to near dry, water (50 mL) was added and the mixture was filtered, the solid was washed with water and cooled ethanol (20 ml) and dried to obtain compound 0108 as a white solid (8.50 g, 85.4%): LCMS: 543 [M+1]⁺. ¹H NMR (CDCl₃): δ 1.26 (t, J=6 Hz, 3H), 3.45-3.54 (m, 2H), 5.06 (s, 2H), 5.11 (s, 2H), 6.61 (s, 1H), 6.73 (t, J=6 Hz, 1H), 7.25-7.39 (m, 10H), 7.52 (s, 1H).

### Step 1i: 5-(2,4-Bis(benzyloxy)-5-chlorophenyl)-N-ethyl-4-(4-hydroxy-phenyl)-isoxazole-3-carboxamide (Compound 0109)

To a mixture of 0102 (1.40 g, 5.53 mmol) and 0108 (2.50, 4.61 mmol) in a mixed solvents of DMF (25 ml) and water (5 ml) was added sodium hydrogen carbonate (1.61 g, 13.83 mmol). To the mixture dichlorobis(triphenylphoshine)Palladium (388 mg, 0.553 mmol) was added and the mixture was heated to 90° C. and stirred overnight. The solvents were removed in vacuo and the residue was partitioned between ethyl acetate and water. And the organic layer was washed with water and brine, dried over anhydrous Na₂SO₄, filtered and evaporated. The residue was purified by column chromatography on silica gel (petroleum ether/ethyl acetate=3/1) to afford product 0109 (2.00 g, 78%): LCMS: 555 [M+1]⁺. ¹H NMR (DMSO-d₆): δ 1.07 (t, J=6 Hz, 3H), 3.18-3.25 (m, 2H), 5.05 (s, 2H), 5.26 (s, 2H), 6.66 (d, J=3 Hz, 2H), 6.98 (d, J=3 Hz, 2H), 7.07-7.10 (m, 3H), 7.29-7.31 (m, 3H), 7.38-7.48 (m, 6H), 8.88 (t, J=3 Hz, 1H), 7.56 (s, 1H).

### Step 1j: Ethyl 4-(4-(5-(2,4-bis(benzyloxy)-5-chlorophenyl)-3-(ethyl-carbamoyl)isoxazol-4-yl)phenoxy)butanoate (Compound 0110-1)

A mixture of 0109 (500 mg, 0.901 mmol), ethyl 4-bromobutanoate (193 mg, 0.991 mmol) and K₂CO₃ (374 mg, 2.703 mmol) in CH₃CN (20 ml) was stirred at 80° C. overnight. After concentrated, the residue was extracted with ethyl acetate. The organic layer was washed with water and brine, dried over anhydrous Na₂SO₄, filtered, evaporated. The solid was washed with cold ethanol to give compound 0110-1 as a white solid (480 mg, 80%): LCMS: 669 [M+1]. ¹H NMR (DMSO-d₆): δ 1.14-1.20 (m, 6H), 1.94 (t, J=6 Hz, 2H), 2.45 (t, J=6 Hz, 2H), 3.20-3.27 (m, 2H), 3.97 (t, J=6 Hz, 2H), 5.03 (s, 2H), 5.26 (s, 2H), 6.84 (d, J=9 Hz, 2H), 7.05-7.11 (m, 5H), 7.28-7.30 (m, 3H), 7.36-7.47 (m, 6H), 8.89 (t, J=6 Hz, 1H).

### Step 1k: Ethyl 4-(4-(5-(5-chloro-2,4-dihydroxyphenyl)-3-(ethyl-carbamoyl)isoxazol-4-yl)phenoxy)butanoate (Compound 0111-1)

To an ice bath cooled solution of compound 0110-1 (850 mg, 1.27 mmol) in dichloromethane (16 ml) under N₂ was added a 1.0 M solution of boron dichloromethane in dichloromethane (5.08 ml, 5.08 mmol). The reaction mixture was stirred at 0° C. for 15 min then warmed to room temperature and stirred for additional 35 min. The reaction mixture was cooled to 0° C. and the reaction was quenched by addition of saturated aqueous sodium hydrogen carbonate solution (16 ml). After stirred for 5 min the dichloromethane was removed in vacuo and the residue was partitioned between ethyl acetate (120 ml) and water (60 ml). The organic phase was washed with water and brine, dried over anhydrous Na₂SO₄, evaporated and the residue was purified by column chromatography on silica gel (petroleum ether/ethyl acetate=2/1) to afford 0111-1 (205 mg, 33%): LCMS: 489 [M+1].

### Step 1l: 5-(5-Chloro-2,4-dihydroxyphenyl)-N-ethyl-4-(4-(4-(hydroxyamino)-4-oxobutoxy)phenyl)isoxazole-3-carboxamide (Compound 1)

Preparation of hydroxylamine in methanol solution: hydroxylamine hydrochloride (4.67 g, 67 mmol) was dissolved in methanol (24 mL) to form solution A. Potassium hydroxide (5.61 g, 100 mmol) was dissolved in methanol (14 mL) to form solution B. The solution A was cooled to 0° C., and solution B was added into solution A dropwise. The mixture was stirred for 30 minutes at 0° C., and the precipitate was filtered off and the filtrate to afford the solution of hydroxylamine in methanol.

To a flask containing compound 0111-1 (200 mg, 0.41 mmol) was added the solution of hydroxylamine in methanol (4.0 ml). The mixture was stirred at room temperature for 30 min. Then it was adjusted to pH4 with 1.2 M hydrochloric acid. The mixture was concentrated and the residue was dissolved in ethyl acetate (200 ml). The organic layer was washed with water, dried over anhydrous Na₂SO₄, concentrated. The residue was purified by column chromatography on silica gel (ethyl acetate) to afford the compound 1 as a white solid (96 mg, 49%): LCMS: 476 [M+1]⁺. ¹H NMR (DMSO-d₆): δ 1.06 (t, J=6 Hz, 3H), 1.87-1.96 (m, 2H), 2.12 (t, J=6 Hz, 2H), 3.19-3.28 (m, 2H), 3.92 (t, J=6 Hz, 2H), 6.57 (s, 1H), 6.84 (d, J=9 Hz, 2H), 7.10-7.15 (m, 3H), 8.68 (s, 1H), 8.85 (t, J=6 Hz, 1H), 10.07 (s, 1H), 10.40 (s, 1H), 10.60 (s, 1H).

### Example 2

### Preparation of 5-(5-Chloro-2,4-dihydroxyphenyl)-N-ethyl-4-(4-(5-(hydroxyamino)-5-oxopentyloxy)phenyl)isoxazole-3-carboxamide (Compound 2)

### Step 2a: Ethyl 5-(4-(5-(2,4-bis(benzyloxy)-5-chlorophenyl)-3-(ethylcarbamoyl)isoxazol-4-yl)phenoxy)pentanoate (Compound 0110-2)

The title compound 0110-2 was prepared (320 mg, 52%) from 0109 (500 mg, 0.90 mmol) and ethyl 5-bromopentanoate (226 mg, 1.08 mmol) using a procedure similar to that described for compound 0110-1 (Example 1): LCMS: 683 [M+1]⁺.

### Step 2b: Ethyl 5-(4-(5-(5-chloro-2,4-dihydroxyphenyl)-3-(ethyl-carbamoyl)isoxazol-4-yl)phenoxy)pentanoate (Compound 0111-2)

The title compound 0111-2 was prepared (81 mg, 37%) from 0110-2 (296 mg, 0.44 mmol) using a procedure similar to that described for compound 0110-1 (Example 1): LCMS: 503 [M+1]⁺.

### Step 2c: 5-(5-Chloro-2,4-dihydroxyphenyl)-N-ethyl-4-(4-(5-(hydroxyamino)-5-oxopentyloxy)phenyl)isoxazole-3-carboxamide (Compound 2)

The title compound 2 was prepared (50 mg, 64%) from compound 0111-2 (81 mg, 0.16 mmol) using a procedure similar to that described for compound 1 (Example 1): LCMS: 490 [M+1]⁺. ¹H NMR (DMSO-d₆): δ 1.08 (t, J=6 Hz, 3H), 1.66 (s, 4H), 2.00 (t, J=6 Hz, 2H), 3.19-3.28 (m, 2H), 3.93 (t, J=6 Hz, 2H), 6.59 (s, 1H), 6.86 (d, J=9 Hz, 2H), 7.12-7.16 (m, 3H), 8.68 (s, 1H), 8.85 (t, J=6 Hz, 1H), 10.08 (s, 1H), 10.40 (s, 1H), 10.60 (s, 1H).

### Example 3

### Preparation of 5-(5-Chloro-2,4-dihydroxyphenyl)-N-ethyl-4-(4-(6-(hydroxyamino)-6-oxohexyloxy)phenyl)isoxazole-3-carboxamide (Compound 3)

### Step 3a: Ethyl 6-(4-(5-(2,4-bis(benzyloxy)-5-chlorophenyl)-3-(ethylcarbamoyl)isoxazol-4-yl)phenoxy)hexanoate (Compound 0110-3)

The title compound 0110-3 was prepared (800 mg, 66%) from 0109 (1.00 g, 1.80 mmol) and ethyl 6-bromohexanoate (0.44 g, 1.97 mmol) using a procedure similar to that described for compound 0110-1 (Example 1): LCMS: 697 [M+1]⁺.

### Step 3b: Ethyl 6-(4-(5-(5-chloro-2,4-dihydroxyphenyl)-3-(ethyl-carbamoyl)isoxazol-4-yl)phenoxy)hexanoate (0111-3)

The title compound 0111-3 was prepared (300 mg, 58%) from 0110-3 (700 mg, 1.0 mmol) using a procedure similar to that described for compound 0110-1 (Example 1): LCMS: 517 [M+1]⁺.

### Step 3c: 5-(5-Chloro-2,4-dihydroxyphenyl)-N-ethyl-4-(4-(6-(hydroxyamino)-6-oxohexyloxy)phenyl)isoxazole-3-carboxamide (Compound 3)

The title compound 3 was prepared (80 mg, 32%) from compound 0111-3 (260 mg, 0.5 mmol) using a procedure similar to that described for compound 1 (Example 1): LCMS: 504 [M+1]⁺. ¹H NMR (DMSO-d₆): δ 1.08 (t, J=6 Hz, 3H), 1.32-1.39 (m, 2H), 1.47-1.55 (m, 2H), 1.64-1.69 (m, 2H), 1.94 (t, J=6 Hz, 2H), 3.18-3.26 (m, 2H), 3.90 (t, J=6 Hz, 2H), 6.54 (s, 1H), 6.84 (d, J=9 Hz, 2H), 7.07-7.14 (m, 3H), 8.67 (s, 1H), 8.85 (t, J=6 Hz, 1H), 10.07 (s, 1H), 10.34 (s, 1H), 10.61 (s, 1H).

### Example 4

### Preparation of 5-(5-chloro-2,4-dihydroxyphenyl)-N-ethyl-4-(4-(7-(hydroxylamino)-7-oxoheptyloxy)phenyl)isoxazole-3-carboxamide (Compound 4)

### Step 4a: Ethyl 7-(4-(5-(2,4-bis(benzyloxy)-5-chlorophenyl)-3-(ethyl-carbamoyl)isoxazol-4-yl)phenoxy)heptanoate (Compound 0110-4)

The title compound 0110-4 was prepared (1.0 g, 78%) from 0109 (1.0 g, 1.8 mmol) and ethyl 7-bromoheptanoate (510 mg, 2.15 mmol) using a procedure similar to that described for compound 0110-1 (Example 1): LCMS: 710 [M+1]⁺.

### Step 4b: Ethyl 7-(4-(5-(5-chloro-2,4-dihydroxyphenyl)-3-(ethyl-carbamoyl)isoxazol-4-yl)phenoxy)heptanoate (Compound 0111-4)

The title compound 0111-4 was prepared (0.82 g, 91.6%) from 0110-4 (1.0 g, 1.4 mmol) using a procedure similar to that described for compound 0110-1 (Example 1): LCMS: 531 [M+1]⁺.

### Step 4c: 5-(5-Chloro-2,4-dihydroxyphenyl)-N-ethyl-4-(4-(7-(hydroxylamino)-7-oxoheptyloxy)phenyl)isoxazole-3-carboxamide (compound 4)

The title compound 4 was prepared (120 mg, 15%) from compound 0111-4 (800 mg, 1.5 mmol) using a procedure similar to that described for compound 1 (Example 1): LCMS: 518 [M+1]⁺. ¹H NMR (DMSO-d₆): δ 1.08 (t, J=6 Hz, 3H), 1.23-1.31 (m, 2H), 1.32-1.39 (m, 2H), 1.47-1.55 (m, 2H), 1.64-1.69 (m, 2H), 1.93 (t, J=6 Hz, 2H), 3.21-3.27 (m, 2H), 3.92 (t, J=6 Hz, 2H), 6.59 (s, 1H), 6.86 (d, J=9 Hz, 2H), 7.10-7.16 (m, 3H), 8.65 (s, 1H), 8.85 (t, J=6 Hz, 1H), 10.07 (s, 1H), 10.34 (s, 1H), 10.61 (s, 1H).

### Example 5

### Preparation of 5-(5-chloro-2,4-dihydroxyphenyl)-N-(3-(hydroxylamino)-3-oxopropyl)-4-(4-methoxyphenyl)isoxazole-3-carboxamide (Compound 5)

### Step 5a: Ethyl 5-(2,4-bis(benzyloxy)-5-chlorophenyl)-4-bromo-isoxazole-3-carboxylate (Compound 0201)

To a suspension of compound 0106 (6.26 g, 13.49 mmol) and potassium acetate (2.80 g, 29.76 mmol) in acetic acid (93 ml) was added a solution of bromine in acetic acid (0.6 M, 225 ml, 134.9 mmol) at room temperature and stirred for 5 min. To the mixture was added saturated aqueous Na₂SO₃. After concentrated, water (50 ml) was added, filtered. The solid was washed with water and cooled ethanol (20 ml) and dried under vacuo to obtain compound 0201 as a white solid (5.8 g, 79%): LCMS: 544 [M+1]⁻. ¹H NMR (DMSO-d₆): δ 1.34 (t, J=6 Hz, 3H), 4.37-4.45 (m, 2H), 5.27 (s, 2H), 5.35 (s, 2H), 7.26 (s, 1H), 7.35-7.51 (m, 10H), 7.65 (s, 1H).

### Step 5b: Ethyl 5-(2,4-bis(benzyloxy)-5-chlorophenyl)-4-(4-methoxyphenyl)isoxazole-3-carboxylate (Compound 0202)

To a mixture of 4-methoxyphenylboronic acid (4.03 g, 26.51 mmol), 0201 (12.1 g, 22.36 mmol), sodium hydrogen carbonate (5.64 g, 67.14 mmol) in a mixed solvents of DMF (25 ml) and water (5 ml) was added dichlorobis(triphenylphoshine)palladium (1.94 mg, 2.76 mmol). The mixture was heated to 90° C. and stirred overnight. The solvent was removed in vacuo and the residue was partitioned between ethyl acetate and water. The organic layer was washed with water and brine, dried over anhydrous Na₂SO₄, filtered and evaporated to obtain crude product which was purified by column chromatography on silica gel (petroleum ether/ethyl acetate=4/1) to afford product 0202 (8.4 g, 66%). LCMS: 570 [M+1]⁺.

### Step 5c: 5-(2,4-Bis(benzyloxy)-5-chlorophenyl)-4-(4-methoxy-phenyl)isoxazole-3-carboxylic acid (Compound 0203)

To the solution of 0202 (4.21 g, 7.40 mmol) in a mixed solvents of THF (80 ml), H₂O (80 ml) and methanol (80 ml) was added LiOH.H₂O (621 mg, 14.80 mmol). The mixture was stirred at r.t. for 30 min, then it was adjusted to pH 4 with 1.2 M HCl. After organic solvent was evaporated, the residue was extracted with ethyl acetate (100 ml×3). The organic layer was dried over anhydrous Na₂SO₄, filtered and evaporated to obtain compound 0203 as a yellow solid (3.98 g, 99%): LCMS: 542 [M+1]⁺. ¹H NMR (DMSO-d⁶): δ 3.75 (s, 3H), 5.06 (s, 2H), 5.25 (s, 2H), 6.85 (d, J=9 Hz, 2H), 7.08-7.14 (m, 4H), 7.37-7.45 (m, 10H), 11.64 (s, 1H).

### Step 5d: Ethyl 3-(5-(2,4-bis(benzyloxy)-5-chlorophenyl)-4-(4-methoxyphenyl)isoxazole-3-carboxamido)propanoate (Compound 0204-5)

A mixture of BOP (980 mg, 2.21 mmol), compound 0203 (1.00 g, 1.84 mmol) and DIEA (953 mg, 7.38 mmol) in DMF (5 mL) was stirred at room temperature for 30 min. To the mixture ethyl 3-aminopropanoate hydrogen chloride (370 mg, 2.4 mmol) was added. The resulting mixture was stirred at room temperature overnight and the mixture was concentrated in vacuo. The residue was dissolved in ethyl acetate (240 ml) and washed with water (15 ml×3), dried over anhydrous Na₂SO4, filtered and evaporated. The residue was purified by column chromatography on silica gel (petroleum ether/ethyl acetate=4/1) to afford the desired product 0204-5 (700 mg, 29%): LCMS: 641 [M+1]⁺.

### Step 5e: Ethyl 3-(5-(5-chloro-2,4-dihydroxyphenyl)-4-(4-methoxy-phenyl)isoxazole-3-carboxamido)propanoate (Compound 0205-5)

To an ice bath cooled solution of compound 0204-5 (690 mg, 1.08 mmol) in dichloromethane (14 ml) under N₂ was added a 1.0 M solution of Boron dichloromethane in dichloromethane (3.3 ml, 3.3 mmol). The reaction mixture was stirred at 0° C. for 15 min then at room temperature for 35 min. The reaction mixture was cooled to 0° C. and quenched by addition of saturated aqueous sodium hydrogen carbonate solution (14 ml). After stirred for 5 min, the solvent was removed in vacuo and the residue was partitioned between ethyl acetate (120 ml) and water (60 ml). The organic phase was washed water and brine, dried over anhydrous Na₂SO₄, filtered and evaporated. The residue was purified by column chromatography on silica gel (petroleum ether/ethyl acetate=2/1) to afford product 0205-5 (350 mg, 70%): LCMS: 461 [M+1]⁺. ¹H NMR (DMSO-d₆): δ 1.20 (t, J=6 Hz, 3H), 2.56 (t, J=6 Hz, 2H), 3.46-3.50 (m, 2H), 3.75 (s, 3H), 4.06 (q, J=6 Hz, 3H), 6.61 (s, 1H), 6.88 (d, J=9 Hz, 2H), 7.14-7.19 (m, 3H), 8.93 (t, J=6 Hz, 1H), 10.08 (s, 1H), 10.61 (s, 1H).

### Step 5f: 5-(5-Chloro-2,4-dihydroxyphenyl)-N-(3-(hydroxylamino)-3-oxo propyl)-4-(4-methoxyphenyl)isoxazole-3-carboxamide (Compound 5)

The title compound 5 was prepared as a brown solid (80 mg, 24%) from compound 0205-5 (340 mg, 0.74 mmol) using a procedure similar to that described for compound 1 (Example 1): LCMS: 448 [M+1]⁺. ¹H NMR (DMSO-d₆): δ 2.28 (t, J=6 Hz, 2H), 3.44 (t, J=6 Hz, 2H), 3.78 (s, 3H), 6.57 (s, 1H), 6.88-6.92 (m, 2H), 7.11-7.18 (m, 3H), 8.88 (t, J=6 Hz, 1H), 10.44 (s, 1H).

### Example 6

### Preparation of 5-(5-chloro-2,4-dihydroxyphenyl)-N-(4-(hydroxyl amino)-4-oxobutyl)-4-(4-methoxyphenyl)isoxazole-3-carboxamide (Compound 6)

### Step 6a: Methyl 4-(5-(2,4-bis(benzyloxy)-5-chlorophenyl)-4-(4-methoxyphenyl)isoxazole-3-carboxamido)butanoate (Compound 0204-6)

The title compound 0204-6 was prepared (442 mg, 37%) from 0203 (1.00 mg, 1.84 mmol) and methyl 4-aminobutanoate hydrogen chloride (368 mg, 2.40 mmol) using a procedure similar to that described for compound 0204-5 (Example 5): LCMS: 641 [M+1]⁻.

### Step 6b: Methyl 4-(5-(5-chloro-2,4-dihydroxyphenyl)-4-(4-methoxyphenyl)isoxazole-3-carboxamido)butanoate (Compound 0205-6)

The title compound 0205-6 was prepared (233 mg, 73%) from 0204-6 (442 mg, 0.69 mmol) using a procedure similar to that described for compound 0205-5 (Example 5): LCMS: 461 [M+1]⁺.

### Step 6c: 5-(5-chloro-2,4-dihydroxyphenyl)-N-(4-(hydroxyamino)-4-oxobutyl)-4-(4-methoxyphenyl)isoxazole-3-carboxamide (Compound 6)

The title compound 6 was prepared (100 mg, 42%) from compound 0205-6 (233 mg, 0.51 mmol) using a procedure similar to that described for compound 1 (Example 1): LCMS: 462 [M+1]⁺. ¹H NMR (DMSO-d₆): δ1.65-1.75 (m, 2H), 1.97 (t, J=6 Hz, 2H), 3.15-3.22 (m, 2H), 3.73 (s, 3H), 6.59 (s, 1H), 6.87 (d, J=9 Hz, 2H), 7.12-7.17 (m, 3H), 8.71 (s, 1H), 8.90 (t, J=6 Hz, 1H), 10.08 (s, 1H), 10.37 (s, 1H), 10.60 (s, 1H).

### Example 7

### Preparation of 5-(5-chloro-2,4-dihydroxyphenyl)-N-(6-(hydroxyl amino)-6-oxohexyl)-4-(4-methoxyphenyl)isoxazole-3-carboxamide (Compound 8)

### Step 7a: Methyl 6-(5-(2,4-bis(benzyloxy)-5-chlorophenyl)-4-(4-methoxyphenyl)isoxazole-3-carboxamido)hexanoate (Compound 0204-8)

The title compound 0204-8 was prepared (500 mg, 41%) from 0203 (1.00 mg, 1.84 mmol) and methyl 6-aminohexanoate hydrogen chloride (503 mg, 2.40 mmol) using a procedure similar to that described for compound 0204-5 (Example 5): LCMS: 669 [M+1]. ¹H NMR (DMSO-d₆): δ 1.43-1.56 (m, 4H), 2.27 (t, J=6 Hz, 2H), 3.15-3.22 (m, 2H), 3.58 (s, 3H), 3.74 (s, 3H), 5.04 (s, 2H), 5.26 (s, 2H), 6.59 (s, 1H), 6.84 (d, J=9 Hz, 2H), 7.06-7.10 (m, 4H), 7.29 (t, J=3 Hz, 3H), 7.38-7.47 (m, 7H), 8.88 (t, J=6 Hz, 1H).

### Step 7b: Methyl 6-(5-(5-chloro-2,4-dihydroxyphenyl)-4-(4-methoxy-phenyl)isoxazole-3-carboxamido)hexanoate (Compound 0205-8)

The title compound 0205-8 was prepared (216 mg, 59%) from 0204-8 (500 mg, 0.75 mmol) using a procedure similar to that described for compound 0205-5 (Example 5): LCMS: 489 [M+1]⁺. ¹H NMR (DMSO-d₆): δ 1.43-1.56 (m, 4H), 2.25 (t, J=6 Hz, 2H), 3.15-3.22 (m, 2H), 3.58 (s, 3H), 3.73 (s, 3H), 6.59 (s, 1H), 6.87 (d, J=9 Hz, 2H), 7.12-7.17 (m, 3H), 8.84 (t, J=6 Hz, 1H), 10.08 (s, 1H), 10.60 (s, 1H).

### Step 7c: 5-(5-Chloro-2,4-dihydroxyphenyl)-N-(6-(hydroxyl-amino)-6-oxohexyl)-4-(4-methoxyphenyl)isoxazole-3-carboxamide (Compound 8)

The title compound 8 was prepared (100 mg, 50%) from compound 0205-8 (200 mg, 0.41 mmol) using a procedure similar to that described for compound 1 (Example 1): LCMS: 490 [M+1]. ¹H NMR (DMSO-d₆): δ1.43-1.53 (m, 4H), 1.93 (t, J=6 Hz, 2H), 3.15-3.22 (m, 2H), 3.73 (s, 3H), 6.59 (s, 1H), 6.87 (d, J=9 Hz, 2H), 7.12-7.17 (m, 3H), 8.66 (s, 1H), 8.84 (t, J=6 Hz, 1H), 10.08 (s, 1H), 10.33 (s, 1H), 10.60 (s, 1H).

### Example 8

### Preparation of 5-(5-chloro-2,4-dihydroxyphenyl)-N-(7-(hydroxyl amino)-7-oxoheptyl)-4-(4-methoxyphenyl)isoxazole-3-carboxamide (Compound 9)

### Step 8a: Ethyl 7-(5-(2,4-bis(benzyloxy)-5-chlorophenyl)-4-(4-methoxyphenyl)isoxazole-3-carboxamido)heptanoate (Compound 0204-9)

The title compound 0204-9 was prepared (640 mg, 52%) from 0203 (1.00 mg, 1.84 mmol) and methyl 7-aminoheptanoate hydrogen chloride (503 mg, 2.40 mmol) using a procedure similar to that described for compound 0204-5 (Example 5): LCMS: 697 [M+1]⁻.

### Step 8b: Ethyl 7-(5-(5-chloro-2,4-dihydroxyphenyl)-4-(4-methoxyphenyl)isoxazole-3-carboxamido)heptanoate (Compound 0205-9)

The title compound 0205-9 was prepared (274 mg, 62%) from 0204-9 (600 mg, 0.86 mmol) using a procedure similar to that described for compound 0205-5 (Example 5): LCMS: 517 [M+1]⁺.

### Step 8c: 5-(5-Chloro-2,4-dihydroxyphenyl)-N-(7-(hydroxyl-amino)-7-oxoheptyl)-4-(4-methoxyphenyl)isoxazole-3-carboxamide (Compound 9)

The title compound 9 was prepared (90 mg, 34%) from compound 0205-9 (90 mg, 34%) using a procedure similar to that described for compound 1 (Example 1): LCMS: 504 [M+1]⁺. ¹H NMR (DMSO-d₆): δ1.22 (s, 4H), 1.43-1.49 (m, 4H), 1.92 (t, J=6 Hz, 2H), 3.13-3.20 (m, 2H), 3.71 (s, 3H), 6.57 (s, 1H), 6.87 (d, J=9 Hz, 2H), 7.10-7.15 (m, 3H), 8.84 (t, J=6 Hz, 1H), 10.06 (s, 1H), 10.30 (s, 1H), 10.58 (s, 1H).

### Example 9

### Preparation of 5-(5-chloro-2,4-dihydroxyphenyl)-N-(8-(hydroxyl amino)-8-oxooctyl)-4-(4-methoxyphenyl)isoxazole-3-carboxamide (Compound 10)

### Step 9a: Methyl 8-(5-(2,4-bis(benzyloxy)-5-chlorophenyl)-4-(4-methoxyphenyl)isoxazole-3-carboxamido)octanoate (Compound 0204-10)

The title compound 0204-10 was prepared (450 mg, 44%) from 0203 (800 mg, 1.48 mmol) and methyl 8-aminooctanoate hydrogen chloride (400 mg, 1.91 mmol) using a procedure similar to that described for compound 0204-5 (Example 5): LCMS: 697 [M+1]⁺.

### Step 9b: Methyl 8-(5-(5-chloro-2,4-dihydroxyphenyl)-4-(4-methoxyphenyl)isoxazole-3-carboxamido)octanoate (Compound 0205-10)

The title compound 0205-10 was prepared (274 mg, 62%) from 0204-10 (450 mg, 0.65 mmol) using a procedure similar to that described for compound 0205-5 (Example 5): LCMS: 517 [M+1]⁺.

### Step 9c: 5-(5-Chloro-2,4-dihydroxyphenyl)-N-(8-(hydroxyamino)-8-oxooctyl)-4-(4-methoxyphenyl)isoxazole-3-carboxamidemn (Compound 10)

The title compound 10 was prepared (70 mg, 71%) from compound 0205-10 (100 mg, 0.19 mmol) using a procedure similar to that described for compound 1 (Example 1): LCMS: 518 [M+1]⁺. ¹H NMR (DMSO-d₆): δ1.23 (s, 6H), 1.43-1.49 (m, 4H), 1.93 (t, J=6 Hz, 2H), 3.15-3.20 (m, 2H), 3.73 (s, 3H), 6.59 (s, 1H), 6.87 (d, J=9 Hz, 2H), 7.12-7.17 (m, 3H), 8.64 (s, 1H), 8.84 (t, J=6 Hz, 1H), 10.08 (s, 1H), 10.33 (s, 1H), 10.60 (s, 1H).

### Biological Assays:

As stated hereinbefore the derivatives defined in the present invention possess anti-proliferation activity. These properties may be assessed, for example, using one or more of the procedures set out below:

(a) An In Vitro Assay which Determines the Ability of a Test Compound to Inhibit Hsp90 Chaperone Activity.

The Hsp90 chaperone assay was performed to measure the ability of HSP90 protein to refold the heat-denatured luciferase protein. HSP90 was first incubated with different concentrations of test compounds in denaturation buffer (25 mM Tris, pH7.5, 8 mM MgSO4, 0.01% bovine gamma globulin and 10% glycerol) at room temperature for 30 min. Luciferase protein was added to denaturation mix and incubated at 50° C. for 8 min. The final concentration of HSP90 and luciferase in denaturation mixture were 0.375 μM and 0.125 μM respectively. A 5 μl sample of the denatured mix was diluted into 25 μl of renaturation buffer (25 mM Tris, pH7.5, 8 mM MgSO4, 0.01% bovine gamma globulin and 10% glycerol, 0.5 mM ATP, 2 mM DTT, 5 mM KCl, 0.3 μM HSP70 and 0.15 μM HSP40). The renaturation reaction was incubated at room temperature for 150 min, followed by dilution of 10 μl of the renatured sample into 90 μl of luciferin reagent (Luclite, PerkinElmer Life Science). The mixture was incubated at dark for 5 min before reading the luminescence signal on a TopCount plate reader (PerkinElmer Life Science).

### (b) HSP90 Competition Binding (Fluorescence Polarization) Assay.

A fluorescein isothiocyanate (FITC) labeled GM was purchase from InvivoGen (ant-fgl-1). The interaction between HSP90 and labeled GM forms the basis for the fluorescence polarization assay. A free and fast-tumbling FITC labeled GM emits random light with respect to the plane of polarization plane of excited light, resulting in a lower polarization degree (mP) value. When GM is bound to HSP90, the complex tumble slower and the emitted light is polarized, resulting in a higher mP value. This competition binding assay was performed in 96-well plate and with each assay contained 10 and 50 nM of labeled GM and purified HSP90 protein (Assay Design, SPP-776F) respectively. The assay buffer contained 20 mM HEPES (pH 7.3), 50 mM KCl, 1 mM DTT, 50 mM MgCl₂, 20 mM Na₂MoO₄, 0.01% NP40 with 0.1 mg/ml bovine gamma-globulin. Compounds are diluted in DMSO and added to the final assay before labeled GM with concentration range from 20 uM to 2 nM. mP value was determined by BioTek Synergy II with background subtraction after 24 hours of incubation at 4° C.

(c) An In Vitro Assay which Determines the Ability of a Test Compound to Inhibit HDAC Enzymatic Activity.

HDAC inhibitors were screened using an HDAC fluorimetric assay kit (AK-500, Biomol, Plymouth Meeting, Pa.). Test compounds were dissolved in dimethylsulphoxide (DMSO) to give a 20 mM working stock concentration. Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm. Each assay was setup as follows: Defrosted all kit components and kept on ice until use. Diluted HeLa nuclear extract 1:29 in Assay Buffer (50 mM Tris/Cl, pH 8.0, 137 mM NaCl, 2.7 mM KCl, 1 mM MgCl2). Prepared dilutions of Trichostatin A (TSA, positive control) and tested compounds in assay buffer (5× of final concentration). Diluted Fluor de Lys™ Substrate in assay buffer to 100 uM (50 fold=2× final). Diluted Fluor de Lys™ developer concentrate 20-fold (e.g. 50 μl plus 950 μl Assay Buffer) in cold assay buffer. Second, diluted the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Added Assay buffer, diluted trichostatin A or test inhibitor to appropriate wells of the microtiter plate. Added diluted HeLa extract or other HDAC sample to all wells except for negative controls. Allowed diluted Fluor de Lys™ Substrate and the samples in the microtiter plate to equilibrate to assay temperature (e.g. 25 or 37° C. Initiated HDAC reactions by adding diluted substrate (25 μl) to each well and mixing thoroughly. Allowed HDAC reactions to proceed for 1 hour and then stopped them by addition of Fluor de Lys™ Developer (50 μl). Incubated plate at room temperature (25° C.) for 10-15 min. Read samples in a microtiter-plate reading fluorimeter capable of excitation at a wavelength in the range 350-380 nm and detection of emitted light in the range 440-460 nm.

The following TABLE 8-B lists compounds representative of the invention and their activity in HDAC and HSP90 assays. In these assays, the following grading was used: I≧10 μM, 10 μM>II>1 μM, 1 μM>III>0.1 μM, and IV≦0.1 μM for IC₅₀.

TABLE 8-B

| Compound No. | HDAC | HSP90 |
|---|---|---|
| 1 | II | IV |
| 2 | III | |
| 3 | III | |
| 4 | III | |
| 5 | III | |

TABLE 9-A
SECTION 9:

| | |
|---|---|
| | (XI) |
| 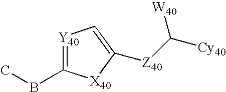 | |

| Compound # | Structure |
|---|---|
| 1 | 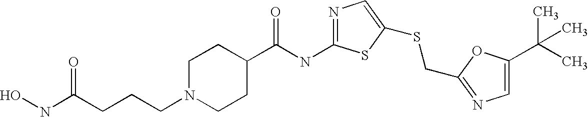 |
| 2 | 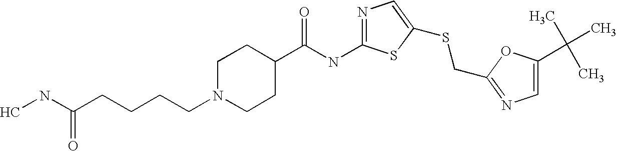 |
| 3 | 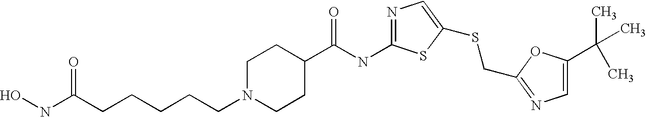 |
| 4 | 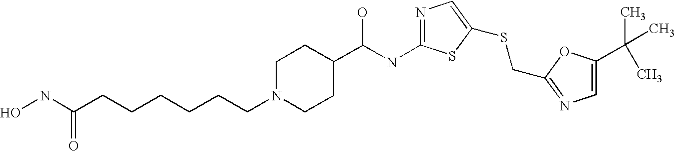 |
| 5 | 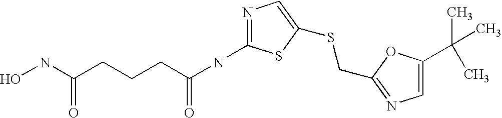 |
| 6 | 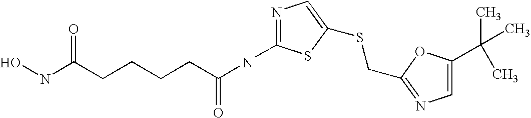 |
| 7 | 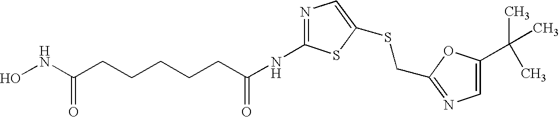 |
| 8 | 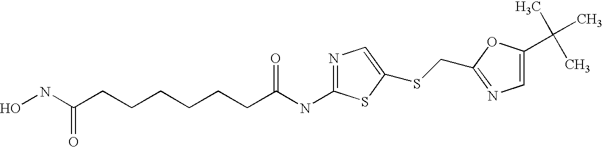 |
| 9 | 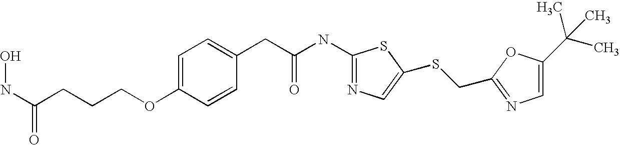 |
| 10 | 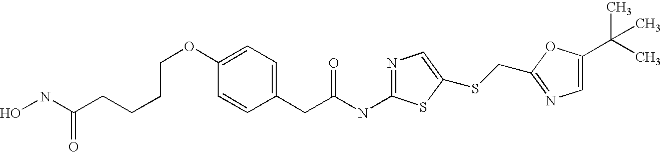 |
| 11 | 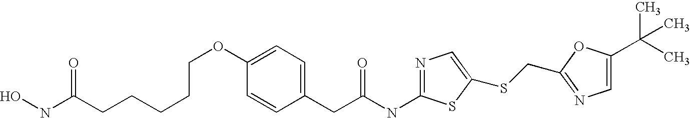 |
| 12 | 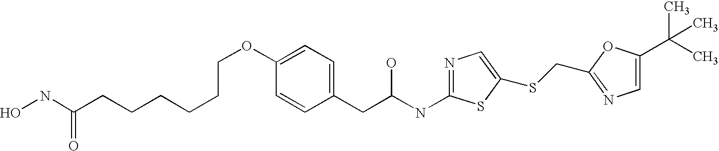 |
| 13 | 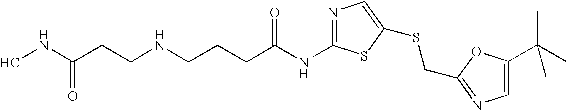 |
| 14 | 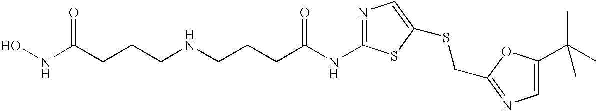 |
| 15 | 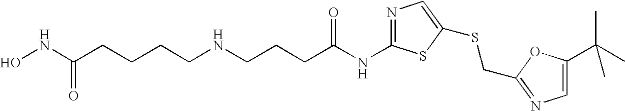 |
| 16 | 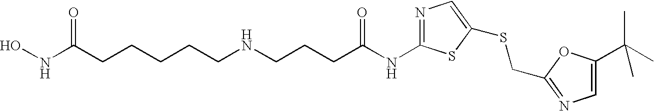 |
| 17 | 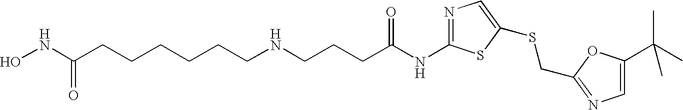 |

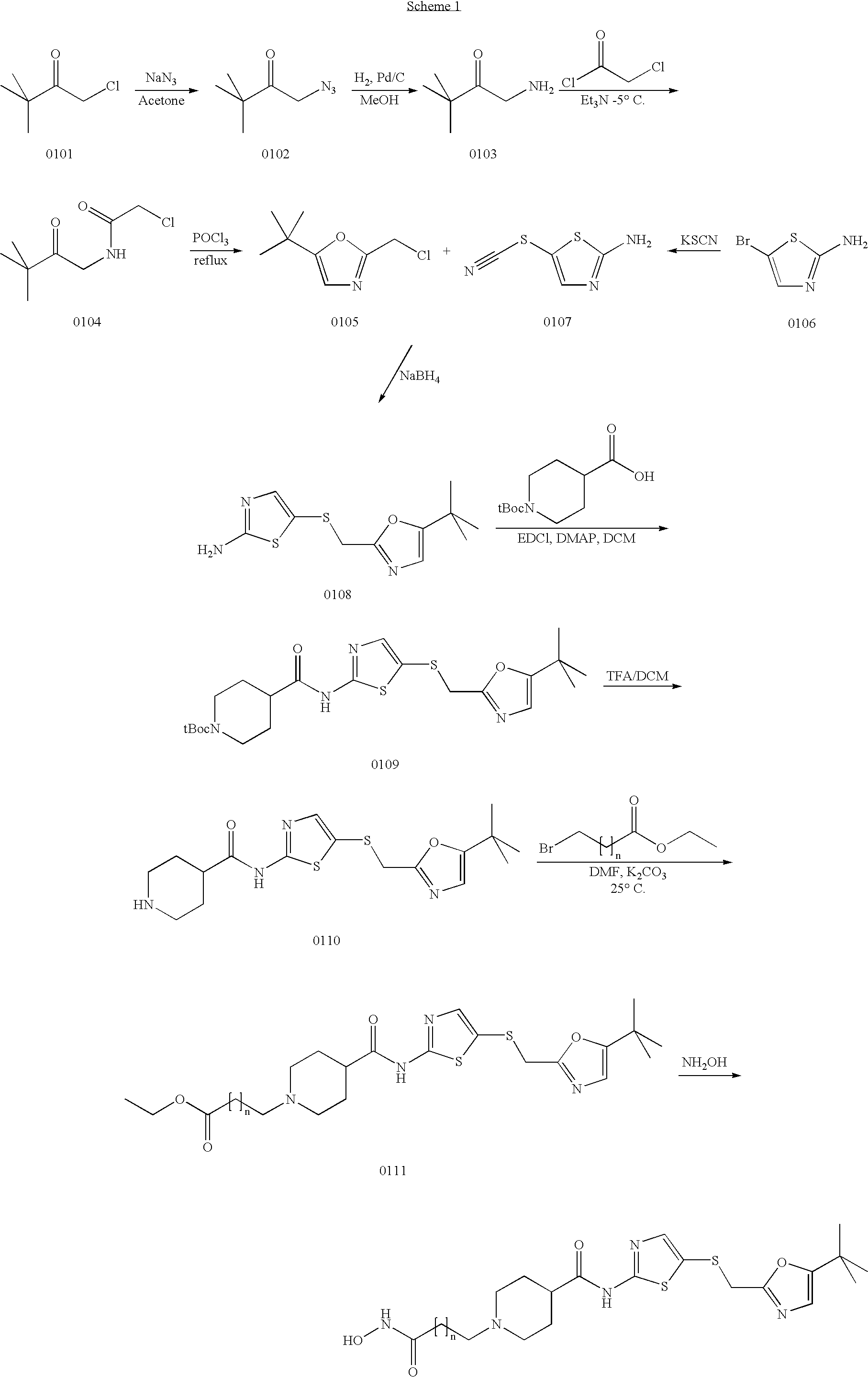

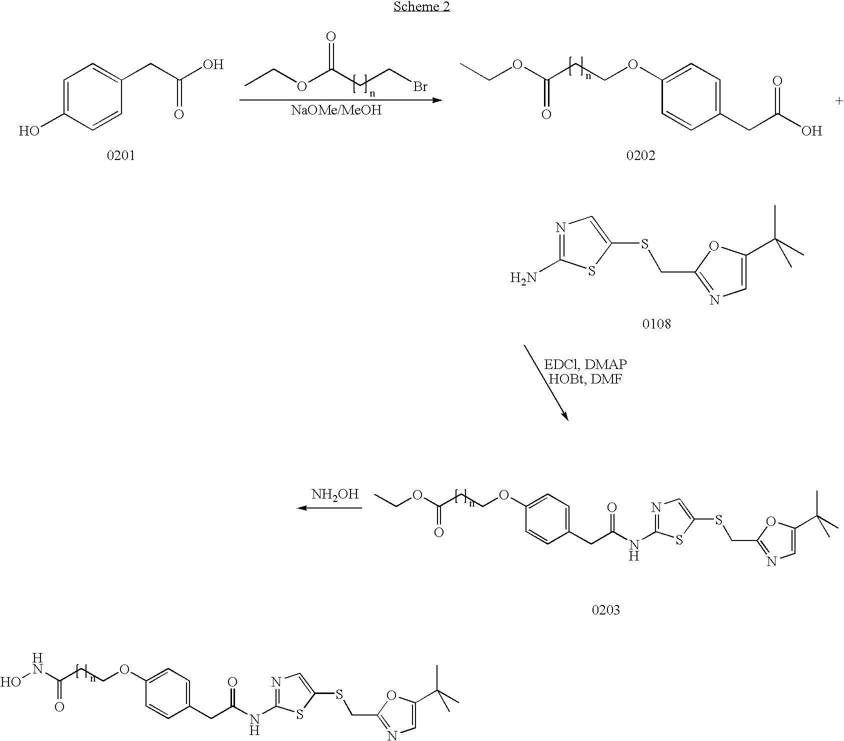

### Example 1

### Preparation of N-(5-((5-tert-butyloxazol-2-yl)methylthio)thiazol-2-yl)-1-(4-(hydroxyamino)-4-oxobutyl)piperidine-4-carboxamide (Compound 1)

### Step 1a: α-Azidopinacolone (Compound 0102)

To a 1 L round-bottom flask fitted with a magnetic stirrer was added α-chlorpinacolone 0101 (33.5 g, 0.25 mol), acetone (400 ml), and sodium azide (21.2 g, 0.325 mol). The reaction mixture was stirred at 25° C. overnight and then filtered, and the solids were washed with acetone. The filtrate was concentrated in vacuo to provide the title compound 0102 as an oil (34.3 g, 100%). The crude material was used in the next step directly without further purification. ¹H NMR (CDCl₃): δ1.17 (s, 9H), 4.07 (s, 2H).

### Step 1b: α-Aminopinacolone hydrochloride (Compound 0103)

To a 2 L round-bottom flask fitted with a magnetic stirrer were added compound 0102 (34.3 g, 245 mmol), methanol (1100 ml), concentrated HCl (24 ml), and 10% Pd/C (4.2 g, wet, 40% water). The reaction mixture was stirred under hydrogen atmosphere overnight. The mixture was filtered through a pad of Celite, and rinsed with methanol. The filtrate was concentrated under reduced pressure at a temperature below 40° C. The resulting wet solid was azeotroped with i-propanol (2×100 ml), and then anhydrous ether (100 ml) was added. The mixture was stirred for 5 min. The solid product was collected by filtration, and the cake was washed with diethyl ether and dried in vacuo to give compound 0103 (28.0 g, 91%), ¹H NMR (DMSO-d₆): δ 1.13 (s, 9H), 4.06 (s, 2H), 8.34 (s, 3H).

### Step 1c: α-N-2 (Chloroacetylamino)pinacolone (Compound 0104)

Triethylamine (35 ml, 250 mmol) was added to a cooled solution (−5° C.) of compound 0103 in CH₂Cl₂ (350 ml). To the resulting mixture which had been cooled to −10° C. a solution of α-chloroacetyl chloride (8.8 ml, 110 mmol) in CH₂Cl₂ (20 ml) was added dropwise over 15 min while keeping the reaction temperature below −5° C. The reaction mixture was stirred for 1 h and quenched with 1 N HCl (200 ml). The organic phase was separated and washed with 1 N HCl (200 ml) and water (50 ml), dried (Na₂SO₄), filtered and evaporated to afford compound 0104 as a white solid (18.9 g, 98%): ¹H NMR (CDCl₃): δ 1.21 (s, 9H), 4.09 (s, 2H), 4.30 (s, 2H), 7.35 (s, 1H).

### Step 1d: 5-tert-Butyl-2-chloromethyloxazole (Compound 0105)

To a 100 ml round-bottom flask fitted with a magnetic stirrer were added compound 0104 (9.534 g, 49.9 mmol) and POCl₃ (30 ml). The reaction mixture was heated to 105° C. and stirred for 1 h. After being cooled to room temperature, the reaction mixture was poured carefully into ice. The mixture was extracted with ether for six times. The organic extracts were combined and neutralized to pH 7-8 with saturated sodium bicarbonate. The organic phase was separated and washed successively with saturated sodium bicarbonate, water, and brine, dried (MgSO4), and concentrated in vacuo. The crude material was distilled under reduced pressure to give the title compound 0105 as a colorless oil (7.756 g, 70%): bp. 49° C./0.25 mmHg. ¹H NMR (CDCl₃): δ 1.32 (s, 9H), 4.60 (s, 2H), 6.70 (s, 1H).

### Step 1e: 5-Thiocyanatothiazol-2-amine (Compound 0107)

A mixture of 2-amino-5-bromothiazole hydrobromide 0106 (53.0 g, 0.204 mol) and potassium thiocyanate (78.5 g, 0.808 mol) in methanol (1.4 L) was stirred at room temperature for 20 h. Methanol was evaporated. The residue was added water (180 ml) and adjusted the pH of the solution to pH=12 with 10% NaOH. The resulting solid was filtered to give the title product 0107 as a brown solid (14.0 g, 44%): LCMS: 157 [M+1].

### Step 1f: 5-((5-Tert-butyloxazol-2-yl)methylthio)thiazol-2-amine (Compound 0108)

To a solution of compound 0107 (3.14 g, 20 mmol) in absolute EtOH (200 ml) was added NaBH₄ (1.6 g, 40 mmol) portionwise at room temperature. The mixture was stirred for 1 h, and then acetone (100 ml) was slowly introduced. After 1 h, a solution of compound 0105 (3.5 g, 20 mmol) in EtOH (30 ml) was added, and the resulting dark reaction mixture heated to reflux for 1 h. The resulting mixture was cooled, concentrated in vacuo, and then partitioned between EtOAc and brine. The organic phase was separated, dried (MgSO4), and concentrated in vacuo to give a crude solid. The crude material was triturated with diethyl ether/hexane to provide compound 0108 as a pale red-brown solid (3.1 g, 57%): LCMS: 270 [M+1]⁺.

### Step 1g: Tert-butyl 4-(5-((5-tert-butyloxazol-2-yl)methylthio)thiazol-2-yl-carbamoyl)piperidine-1-carboxylate (Compound 0109)

To a solution of compound 0108 (750 mg, 2.79 mmol), 1-(tert-butoxycarbonyl)piperidine-4-carboxylic acid (960 mg, 4.18 mmol), DMAP (510 mg, 4.18 mmol) in DMF were added EDAC (802 mg, 4.18 mmol) and HOBt (560 mg, 4.18 mmol). The mixture was heated to 50° C. and stirred overnight. The mixture was diluted with EtOAc and washed with brine, aqueous HCl, saturate NaHCO₃ and brine. The organic phase was dried over Na₂SO₄ and purified by column chromatography on silica gel (ethyl acetate/petroleum ether=1:2 to pure ethyl acetate) to afford the title compound 0109 (1.0 g, 74.6%): LCMS: 481 [M+1]⁺.

### Step 1h: N-(5-((5-Tert-butyloxazol-2-yl)methylthio)thiazol-2-yl)piperidine-4-carboxamide (Compound 0110)

To a mixture of compound 0109 (1.0 g, 2 mmol) in dichloromethane (20 ml) was added TFA (2 ml). The reaction mixture was stirred at 30° C. for 3 h. After reaction the mixture was brought to pH 7-8 with saturate NaHCO₃ and exacted with ethyl acetate. The organic phase was dried over Na₂SO₄, concentrated to give the title compound 0110 (620 mg, 82%): mp 178.5-180° C., LCMS: 381 [M+1]⁺, ¹H NMR (CDCl₃): δ 1.164 (s, 9H), 1.720-1.795 (m, 2H), 1.923-1.969 (m, 2H), 2.714-2.777 (m, 1H), 2.889 (t, J=12 Hz, 2H), 3.281 (s, 1H), 4.046 (s, 1H), 6.708 (s, 1H), 7.393 (s, 1H), 8.844 (m, 1H).

### Step 1i: Ethyl 4-(4-(5-((5-tert-butyloxazol-2-yl)methylthio)thiazol-2-ylcarbamoyl)piperidin-1-yl)butanoate (Compound 0111-1)

To a solution of 0110 (300 mg, 0.789 mmol) in DMF (10 ml) was added ethyl 4-bromobutanoate (153 mg, 0.789 mmol). The reaction mixture was stirred at room temperature for 30 min. K₂CO₃ (108 mg, 0.789 mmol) was added to the mixture and the resulting mixture was stirred at room temperature overnight. The mixture was washed with water and extracted with CH₂Cl₂. The organic phase was dried over Na₂SO₄, concentrated to give the crude product. The crude product was purified by column chromatography on silica gel (ethyl acetate/petroleum ether=1:1 to 100% ethyl acetate) to give the title compound 0111 (180 mg, 46%), LCMS: 496 [M+1]⁺.

### Step 1j: N-(5-((5-Tert-butyloxazol-2-yl)methylthio)thiazol-2-yl)-1-(4-(hydroxylamino)-4-oxobutyl)piperidine-4-carboxamide (Compound 1)

The freshly prepared hydroxylamine solution (2.1 ml, 3.6 mmol) was placed in 10 ml flask. Compound 0111 (180 mg, 0.36 mmol) was added to this solution and stirred at 25° C. for 4 hours. The mixture was neutralized with acetic acid, and the methanol was removed. The residue was purified by prep.HPLC to give the title compound 1 as a white solid (25 mg, 14%): mp 176-180° C., LCMS: 482 [M+1]⁺, ¹H NMR (DMSO-d₆): δ 1.149 (s, 9H), 1.352 (m, 2H), 1.720-2.320 (m, 10H), 2.403 (m, 1H), 2.570 (m, 2H), 4.032 (s, 2H), 6.696 (s, 1H), 7.350 (s, 1H), 8.747 (s, 1H), 10.440 (s, 1H), 12.326 (s, 1H).

### Example 2

### Preparation of N-(5-((5-tert-butyloxazol-2-yl)methylthio)thiazol-2-yl)-1-(5-(hydroxyamino)-5-oxopentyl)piperidine-4-carboxamide (Compound 2)

### Step 2a: Methyl 5-(4-(5-((5-tert-butyloxazol-2-yl)methylthio)thiazol-2-ylcarbamoyl)piperidin-1-yl)pentanoate (Compound 0111-2)

The title compound 0111-2 was prepared as a yellow solid (126 mg, 38.7%) from compound 0110 (250 mg, 0.658 mmol), methyl 5-bromopentanoate (128 mg, 0.658 mmol), K₂CO₃ (90.8 mg, 0.658 mmol), and DMF (5 ml) using a procedure similar to that described for compound 0111-1 (Example 1): LCMS: 495 [M+1].

### Step 2b: N-(5-((5-tert-butyloxazol-2-yl)methylthio)thiazol-2-yl)-1-(5-(hydroxyl-amino)-5-oxopentyl)piperidine-4-carboxamide (Compound 2)

The title compound 2 was prepared as a yellow solid (20 mg, 15.8%) from compound 0111-2 (126 mg, 0.255 mmol) and freshly prepared hydroxylamine solution (1.5 ml, 2.55 mmol) using a procedure similar to that described for compound 1 (Example 1): M.p.: 93-97° C.; LCMS: 496 [M+1]⁺. ¹H NMR (DMSO-d₆): δ 1.148 (s, 9H), 1.353-1.949 (m, 10H), 2.187-2.227 (m, 2H), 2.408 (m, 1H), 2.837 (d, J=11.1, 2H), 4.026 (s, 2H), 6.696 (s, 1H), 7.355 (s, 1H), 8.647 (s, 1H), 10.314 (s, 1H), 12.190 (s, 1H).

### Example 3

### Preparation of N-(5-((5-tert-butyloxazol-2-yl)methylthio)thiazol-2-yl)-1-(6-(hydroxyamino)-6-oxohexyl)piperidine-4-carboxamide (Compound 3)

### Step 3a: Ethyl 6-(4-(5-((5-tert-butyloxazol-2-yl)methylthio)thiazol-2-yl-carbamoyl)piperidin-1-yl)hexanoate (Compound 0111-3)

The title compound 0111-3 was prepared as a yellow solid (210 mg, 51%) from compound 0110 (300 mg, 0.789 mmol), ethyl 6-bromohexanoate (176 mg, 0.789 mmol), K₂CO₃ (108 mg, 0.789 mmol) and DMF (5 ml) using a procedure similar to that described for compound 0111-1 (Example 1): LCMS: 523 [M+1]⁺.

### Step 3b: N-(5-((5-Tert-butyloxazol-2-yl)methylthio)thiazol-2-yl)-1-(6-(hydroxyamino)-6-oxohexyl)piperidine-4-carboxamide (Compound 3)

The title compound 3 was prepared as a yellow solid (30 mg, 15.8%) from compound 0111-3 (210 mg, 0.40 mmol) and freshly prepared hydroxylamine solution (2.5 ml, 4.0 mmol) using a procedure similar to that described for compound 1 (Example 1): M.p.: 127-130° C.; LCMS: 510 [M+1]⁺. ¹H NMR (DMSO-d₆): δ 1.158 (s, 9H), 1.218-1.927 (m, 14H), 2.204-2.254 (m, 2H), 2.402 (m, 1H), 2.619 (m, 2H), 4.033 (s, 2H), 6.698 (s, 1H), 7.377 (s, 1H), 8.669 (s, 1H), 10.345 (s, 1H), 12.354 (s, 1H).

### Example 4

### Preparation of N-(5-((5-tert-butyloxazol-2-yl)methylthio)thiazol-2-yl)-1-(7-(hydroxyamino)-7-oxoheptyl)piperidine-4-carboxamide (Compound 4)

### Step 4a: Ethyl 7-(4-(5-((5-tert-butyloxazol-2-yl)methylthio)thiazol-2-ylcarbamoyl)piperidin-1-yl)heptanoate (Compound 0111-4)

The title compound 0111-4 was prepared as a yellow solid (370 mg, 62%) from compound 0110 (423 mg, 1.113 mmol), ethyl 7-bromoheptanoate (260 mg, 1.113 mmol), K₂CO₃ (154 mg, 1.113 mmol) and DMF (5 ml) using a procedure similar to that described for compound 0111-1 (Example 1): LCMS: 537 [M+1]⁺.

### Step 4b: N-(5-((5-tert-butyloxazol-2-yl)methylthio)thiazol-2-yl)-1-(7-(hydroxyl-amino)-7-oxoheptyl)piperidine-4-carboxamide (compound 4)

The title compound 4 was prepared as a yellow solid (20 mg, 6%) from compound 0111-4 (370 mg, 0.69 mmol) and freshly prepared hydroxylamine solution (4.0 ml, 6.9 mmol) using a procedure similar to that described for compound 1 (Example 1): M.p.: 113-115° C.; LCMS: 524 [M+1]; ¹H NMR (DMSO-d₆): δ 1.153 (s, 9H), 1.215-1.483 (m, 4H), 1.545-1.628 (m, 4H), 1.708-1.892 (m, 6H), 1.917-2.224 (m, 4H), 2.425 (m, 1H), 2.844-2.882 (m, 2H), 4.031 (s, 2H), 6.701 (s, 1H), 7.361 (s, 1H), 8.655 (s, 1H), 10.361 (s, 1H), 12.216 (s, 1H).

### Example 5

### Preparation of 4-(4-(2-(5-((5-tert-butyloxazol-2-yl)methylthio)thiazol-2-ylamino)-2-oxoethyl)phenoxy)-N-hydroxybutanamide (Compound 9)

### Step 5a: 2-(4-(4-Methoxy-4-oxobutoxy)phenyl)acetic acid (Compound 0202-9)

To the solution of MeONa (1.08 g, 20 mmol) in MeOH (20 ml) was added compound 0201 (1.52 g, 10 mmol) at 0° C. under nitrogen. The mixture was stirred for 10 minutes and ethyl 4-bromobutanoate (1.94 g, 10 mmol) was added. After stirred at 50° C. overnight, the mixture was adjusted PH 6-7 with acetic acid, and concentrated. The residue was taken up in ethyl acetate, washed with water, brine, dried and concentrated to give a residue which was purified by column chromatography (eluent: ethyl acetate/petroleum ether 1/5) to afford the product 0202-9 as a solid (841 mg, 33%): ¹H NMR (DMSO-d₆): δ 12.24 (s, 1H), 7.15 (d, J=8.7 Hz, 2H), 6.85 (d, J=8.7 Hz, 2H), 3.95 (t, J=6.3 Hz, 2H), 3.59 (s, 3H), 3.47 (s, 2H), 2.49 (m, 2H), 1.95 (m, 2H).

### Step 5b: Methyl4-(4-(2-(5-((5-tert-butyloxazol-2-yl)methylthio)thiazol-2-ylamino)-2-oxoethyl)phenoxy)butanoate (Compound 0203-9)

The solution of 0202-9 (0.189 g, 0.75 mmol), 0108 (0.135 g, 0.5 mmol), EDCI (0.143 g, 0.75 mmol), DMAP (0.092 g, 0.75 mmol), HOBt (0.101 g, 0.75 mmol) in DMF (5 ml) was stirred at 40° C. for 4 hours, After that, the mixture was poured into ethyl acetate (50 ml), and washed with water and brine, dried and concentrated to give a residue which was purified by column chromatography (eluent: ethyl acetate/petroleum ether=1/3) to afford the product 0203-9 as a solid (40 mg, 16%): ¹H NMR (DMSO-d₆): δ 12.43 (s, 1H), 7.39 (s, 1H), 7.19 (d, J=8.7 Hz, 2H), 6.86 (d, J=8.7 Hz, 2H), 6.69 (s, 1H), 4.04 (s, 2H), 3.95 (t, J=6.3 Hz, 2H), 3.65 (s, 2H), 3.59 (s, 3H), 2.50 (t, J=3.3 Hz, 2H), 1.95 (m, 2H), 1.13 (s, 9H).

### Step 5c: 4-(4-(2-(5-((5-Tert-butyloxazol-2-yl)methylthio)thiazol-2-yl-amino)-2-oxoethyl)phenoxy)-N-hydroxybutanamide (Compound 9)

Preparation of hydroxylamine in methanol solution: hydroxylamine hydrochloride (4.67 g, 67 mmol) was dissolved in methanol (24 mL) to form solution A. Potassium hydroxide (5.61 g, 100 mmol) was dissolved in methanol (14 mL) to form solution B. To the solution A at 0° C. was added solution B dropwise. The mixture was stirred for 30 minutes at 0° C., and the solid was filtered to afford a solution of hydroxylamine in methanol.

To a flask containing compound 0203-9 (40 mg, 0.080 mmol) was added the solution of hydroxylamine in methanol (6.0 mL). The mixture was stirred at room temperature for 1 hour. Then it was adjusted PH 7 with concentrated HCl. The mixture was concentrated to give a residue which was washed with water to afford the product 9 as a solid (18 mg, 44% yield). ¹H NMR (DMSO-d₆): δ 12.43 (s, 1H), 10.391 (s, 1H), 8.68 (s, 1H), 7.36 (s, 1H), 7.18 (d, J=8.7 Hz, 2H), 6.84 (d, J=8.7 Hz, 2H), 6.67 (s, 1H), 4.01 (s, 2H), 3.90 (t, J=6 Hz, 2H), 3.63 (s, 2H), 2.11 (t, J=7.2 Hz, 2H), 1.19 (m, 2H), 1.12 (s, 9H).

### Example 6

### Preparation of 5-(4-(2-(5-((5-tert-butyloxazol-2-yl)methylthio)thiazol-2-ylamino)-2-oxoethyl)phenoxy)-N-hydroxypentanamide (Compound 10)

### Step 6a: 2-(4-(5-Methoxy-5-oxopentyloxy)phenyl)acetic acid (Compound 0202-10)

The title compound 0202-10 was prepared as a yellow solid (322 mg, 24%) from compound 0201 (0.76 g, 5 mmol), and methyl 5-bromopentanoate (0.98 g, 5 mmol) using a procedure similar to that described for compound 0202-9 (Example 5): ¹H NMR (DMSO-d₆): δ 12.24 (s, 1H), 7.15 (d, J=8.7 Hz, 2H), 6.91 (d, J=8.7 Hz, 2H), 3.94 (t, J=6.0 Hz, 2H), 3.59 (s, 3H), 3.48 (s, 2H), 2.38 (t, J=7.2 Hz, 2H), 1.69 (m, 4H).

### Step 6b: Methyl-5-(4-(2-(5-((5-tert-butyloxazol-2-yl)methylthio)-thiazol-2-ylamino)-2-oxoethyl)phenoxy)pentanoate (compound 0203-10)

The solution of 0202-10 (0.193 g, 0.75 mmol), 0108 (0.135 g, 0.5 mmol), EDCI (0.143 g, 0.75 mmol), DMAP (0.092 g, 0.75 mmol), HOBt (0.101 g, 0.75 mmol) in DMF (5 ml) was stirred at 40° C. for 4 hours, After that, the mixture was poured into ethyl acetate (50 ml), and washed with water and brine, dried and concentrated to give a residue which was purified by column chromatography (ethyl acetate/petroleum ether=1/3) to afford the product 0203-10 as a solid (45 mg, 12%). ¹H NMR (DMSO-d₆): δ 12.45 (s, 1H), 7.39 (s, 2H), 7.20 (d, J=9.0 Hz, 2H), 6.87 (d, J=9.0 Hz, 2H), 6.70 (s, 1H), 4.04 (s, 2H), 3.93 (t, J=6.3 Hz, 2H), 3.65 (s, 2H), 3.58 (s, 3H), 2.37 (t, J=6.0 Hz, 2H), 1.69 (m, 4H), 1.137 (s, 9H).

### Step 6c: 5-(4-(2-(5-((5-Tert-butyloxazol-2-yl)methylthio)thiazol-2-yl-amino)-2-oxoethyl)phenoxy)-N-hydroxypentanamide (Compound 10)

The title compound 10 was prepared as a yellow solid (17 mg, 38% yield) from compound 0203-10 (45 mg, 0.087 mmol) and freshly prepared solution of hydroxylamine in methanol (6.0 mL) using a procedure similar to that described for compound 9 (Example 5): ¹H NMR (DMSO-d₆): δ 12.43 (s, 1H), 10.36 (s, 1H), 8.69 (s, 1H), 7.38 (s, 1H), 7.20 (d, J=9.0 Hz, 2H), 6.86 (d, J=9.0 Hz, 2H), 6.69 (s, 2H), 4.04 (s, 2H), 3.92 (t, J=6.0 Hz, 2H), 1.99 (t, J=6.0 Hz, 2H), 1.64 (m, 4H), 1.14 (s, 9H).

### Example 7

### Preparation of methyl 6-(4-(2-(5-((5-tert-butyloxazol-2-yl)methyl thio)thiazol-2-ylamino)-2-oxoethyl)phenoxy)hexanoate (Compound 11)

### Step 7a: 2-(4-(6-Methoxy-6-oxohexyloxy)phenyl)acetic acid (compound 0202-11)

The title compound 0202-11 was prepared as a yellow solid (950 mg, 34%) from compound 0201 (0.76 g, 5 mmol), and methyl 5-bromopentanoate (2.22 g, 10 mmol) using a procedure similar to that described for compound 0202-9 (Example 5): ¹H NMR (DMSO-d₆): δ 12.22 (s, 1H), 7.14 (d, J=8.4 Hz, 2H), 6.85 (d, J=8.4 Hz, 2H), 3.92 (t, J=6.3 Hz, 2H), 3.58 (s, 3H), 3.47 (s, 2H), 2.32 (t, J=7.5 Hz, 2H), 1.69 (m, 2H), 1.55 (m, 2H), 1.40 (m, 2H).

### Step 7b: Methyl 6-(4-(2-(5-((5-tert-butyloxazol-2-yl)methylthio)-thiazol-2-ylamino)-2-oxoethyl)phenoxy)hexanoate (Compound 0203-11)

The title compound 0203-11 was prepared as a yellow solid (63 mg, 16%) from compound 0202-9 using a procedure similar to that described for compound 0203-9 (Example 5): ¹H NMR (DMSO-d₆): δ 12.43 (s, 1H), 7.37 (s, 1H), 7.18 (d, J=8.7 Hz, 2H), 6.83 (d, J=8.7 Hz, 2H), 6.67 (s, 1H), 4.02 (s, 2H), 3.89 (t, J=6.3 Hz, 2H), 3.63 (s, 2H), 3.55 (s, 3H), 2.30 (t, J=7.2 Hz, 2H), 1.67 (m, 2H), 1.55 (m, 2H), 1.37 (m, 2H), 1.11 (s, 9H).

### Step 7c: Methyl 6-(4-(2-(5-((5-tert-butyloxazol-2-yl)methylthio)-thiazol-2-ylamino)-2-oxoethyl)phenoxy)hexanoate (Compound 11)

The title compound 11 was prepared as a yellow solid (82 mg, 42% yield) from compound 0203-11 (193 mg, 0.363 mmol) and freshly prepared solution of hydroxylamine in methanol (6.0 mL) using a procedure similar to that described for compound 9 (Example 5): ¹H NMR (DMSO-d₆): δ 12.46 (s, 1H), 10.35 (s, 1H), 8.68 (s, 1H), 7.39 (s, 1H), 7.18 (d, J=8.4 Hz, 2H), 6.85 (d, J=8.4 Hz, 2H), 6.70 (s, 1H), 4.04 (s, 2H), 3.92 (t, J=6.3 Hz, 2H), 3.65 (s, 3H), 1.96 (t, J=6.3 Hz, 2H), 1.69 (m, 2H), 1.54 (m, 2H), 1.37 (m, 2H), 1.143 (s, 9H).

### Example 8

### Preparation of N-hydroxy-7-(4-(2-(5-((5-isopropyloxazol-2-yl)methylthio)thiazol-2-ylamino)-2-oxoethyl)phenoxy)heptanamide (Compound 12)

### Step 8a: 2-(4-(7-Methoxy-7-oxoheptyloxy)phenyl)acetic acid (Compound 0202-12)

The title compound 0202-12 was prepared as a yellow solid (219 mg, 15%) from compound 0201 using a procedure similar to that described for compound 0202-9 (Example 5): ¹H NMR (DMSO-d₆): δ 12.22 (s, 1H), 7.14 (d, J=8.1 Hz, 2H), 6.84 (d, J=8.1 Hz, 2H), 3.89 (t, J=6.3 Hz, 2H), 3.55 (s, 3H), 3.44 (s, 2H), 2.30 (t, J=7.2 Hz, 2H), 1.68 (m, 2H), 1.54 (m, 2H), 1.35 (m, 4H).

### Step 8b: Methyl 7-(4-(2-(5-((5-tert-butyloxazol-2-yl)methylthio)-thiazol-2-ylamino)-2-oxoethyl)phenoxy)heptanoate (Compound 0203-12)

The title compound 0203-12 was prepared as a yellow solid (100 mg, 26%) from compound 0202-12 using a procedure similar to that described for compound 0203-9 (Example 5): ¹H NMR (DMSO-d₆): δ 12.45 (s, 1H), 7.39 (s, 1H), 7.20 (d, J=8.4 Hz, 2H), 6.86 (d, J=8.4 Hz, 2H), 6.69 (s, 1H), 4.04 (s, 2H), 3.91 (t, J=6.3 Hz, 2H), 3.65 (s, 2H), 3.57 (s, 3H), 2.30 (t, J=7.2 Hz, 2H), 1.65 (m, 2H), 1.51 (m, 2H), 1.34 (m, 4H), 1.13 (s, 9H).

### Step 8c: N-Hydroxy-7-(4-(2-(5-((5-isopropyloxazol-2-yl)methylthio)-thiazol-2-ylamino)-2-oxo-ethyl)phenoxy)heptanamide (Compound 12)

The title compound 12 was prepared as a solid (65 mg, 68% yield) from compound 0203-12 (95 mg, 0.174 mmol) and freshly prepared solution of hydroxylamine in methanol (10.0 mL) using a procedure similar to that described for compound 9 (Example 5): ¹H NMR (DMSO-d₆): δ 12.45 (s, 1H), 10.33 (s, 1H), 8.66 (s, 1H), 7.39 (s, 1H), 7.20 (d, J=8.4 Hz, 2H), 6.87 (d, J=8.4 Hz, 2H), 6.69 (s, 1H), 4.04 (s, 2H), 3.91 (t, J=6.3 Hz, 2H), 3.65 (s, 2H), 3.57 (s, 3H), 2.30 (t, J=7.2 Hz, 2H), 1.65 (m, 2H), 1.53 (m, 2H), 1.34 (m, 4H), 1.13 (s, 9H).

### Biological Assays:

As stated hereinbefore the derivatives defined in the present invention possess anti-proliferation activity. These properties may be assessed, for example, using one or more of the procedures set out below:

(a) An In Vitro Assay which Determines the Ability of a Test Compound to Inhibit CDK Activity.

### Materials:

CDK2/cyclinE (Accession number for CDK2; EMBL M68520, for cyclinE1; GenBank NM_{—}001238): C-terminal 6His-tagged, recombinant full-length CDK2 in complex with N-terminal GST-tagged, recombinant full-length cyclinE1. Both are expressed by baculovirus in Sf21 cells. Purified using Ni2+/NTA agarose. Combined purity 76% by SDS-PAGE and Coomassie blue staining. CDK2 MW=34 kDa, cyclinE1 MW=74 kDa. Specific Activity of 1336 U/mg, where one unit of CDK2/cylinE1 activity is defined as 1 nmol phosphate incorporated into 0.1 mg/ml histone H1 per minute at 30° C. with a final ATP concentration of 100 μM. Enzyme at 0.1 mg/ml in 50 mM Tris/HCl pH 7.5, 150 mM NaCl, 0.03% Brij-35, 0.1 mM EGTA, 0.2 mM PMSF, 1 mM benzamidine, 0.1% 2-mercaptoethanol, 270 mM sucrose. CDK6/cyclinD3 (Accession number for CDK6; GenBank X66365, for cyclin D3; EMBL M90814): N-terminal, 6His-tagged full-length human cdk6 complexed with N-terminal GST-tagged full-length human cyclin D3, expressed in Sf21 cells. Purified using glutathione-agarose, activated with CAK, and repurified on Ni2+/NTA-agarose. Purity 68%. MW=38 kDa (cdk6) and 59 kDa (cyclin D3). Specific Activity of 39 U/mg, where one unit of cdk6/cyclinD3 activity is defined as 1 nmol phosphate incorporated into 0.1 mg/ml histone H1 per minute at 30° C. with a final ATP concentration of 100M. Enzyme at 0.1 mg/ml in 50 mM Tris-HCl, pH 7.5, 270 mM sucrose, 150 mM NaCl, 1 mM benzamidine, 0.2 mM PMSF, 0.1% 2-mercaptoethanol, 0.1 mM EGTA, 0.03% Brij 35. Histon H1 (Substrate for CDK2 & 6): Sigma cat#H4524, isolated as a lysine rich fraction from calf thymus, 93% purity, Mw=21.5 kDa, stock at 20 mg/ml=930 μM in DW.
Reaction Buffer: 20 mM HEPES (pH 7.5), 10 mM MgCl₂, 1 mM EGTA, 0.02% Brij 35, 0.02 mg/ml BSA, 0.1 mM Na₃VO₄, 2 mM DTT.
[γ-³³P]-ATP: Perkin Elmer cat#NEG602H1MC (EasyTides), 10 mCi/ml=10 μCi/μl, 100 μl in vial, specific activity=3000 Ci/mmol, 3.3-5 μM in 50 mM Tricine (pH 7.6), amber gold dye.

### Assay Conditions:

CDK2/cyclinE: 0.5 nM CDK2/cyclinE and 5 μM Histon H1 are in the reaction buffer plus 1 μM ATP and 1% DMSO final. Incubate for 2 hours at room temperature. Conversion rate of ATP: 4.5%
CDK6/cyclinD3: 50 nM CDK6/cyclinD3 and 5 μM Histon H1 are in the reaction buffer plus 1 μM ATP and 1% DMSO final. Incubate for 2 hours at room temperature. Conversion rate of ATP: 13%
(b) An In Vitro Assay which Determines the Ability of a Test Compound to Inhibit HDAC Enzymatic Activity.

HDAC inhibitors were screened using an HDAC fluorimetric assay kit (AK-500, Biomol, Plymouth Meeting, Pa.). Test compounds were dissolved in dimethylsulphoxide (DMSO) to give a 20 mM working stock concentration. Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm. Each assay was setup as follows: Defrosted all kit components and kept on ice until use. Diluted HeLa nuclear extract 1:29 in Assay Buffer (50 mM Tris/Cl, pH 8.0, 137 mM NaCl, 2.7 mM KCl, 1 mM MgCl2). Prepared dilutions of Trichostatin A (TSA, positive control) and tested compounds in assay buffer (5× of final concentration). Diluted Fluor de Lys™ Substrate in assay buffer to 100 uM (50 fold=2× final). Diluted Fluor de Lys™ developer concentrate 20-fold (e.g. 50 μl plus 950 μl Assay Buffer) in cold assay buffer. Second, diluted the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Added Assay buffer, diluted trichostatin A or test inhibitor to appropriate wells of the microtiter plate. Added diluted HeLa extract or other HDAC sample to all wells except for negative controls. Allowed diluted Fluor de Lys™ Substrate and the samples in the microtiter plate to equilibrate to assay temperature (e.g. 25 or 37° C. Initiated HDAC reactions by adding diluted substrate (25 μl) to each well and mixing thoroughly. Allowed HDAC reactions to proceed for 1 hour and then stopped them by addition of Fluor de Lys™ Developer (50 μl). Incubated plate at room temperature (25° C.) for 10-15 min. Read samples in a microtiter-plate reading fluorimeter capable of excitation at a wavelength in the range 350-380 nm and detection of emitted light in the range 440-460 nm.

The following TABLE 9-B lists compounds representative of the invention and their activity in HDAC and CDK assays. In these assays, the following grading was used: I≧10 μM, 10 μM>II>1 μM, 1 μM>III>0.1 μM, and IV≦0.1 μM for IC₅₀.

TABLE 9-B

| Compound No. | HDAC | CDK2/cyclinE | CDK6/cyclinD |
|---|---|---|---|
| 1 | I | | |
| 2 | II | | |
| 3 | II | | |
| 4 | III | | |
| 5 | III | IV | III |
| 6 | III | IV | III |
| 7 | IV | IV | III |
| 8 | III | IV | |
| 9 | III | IV | |
| 10 | III | IV | |

TABLE 10-A
SECTION 10:

| | |
|---|---|
| | (XII) |
| 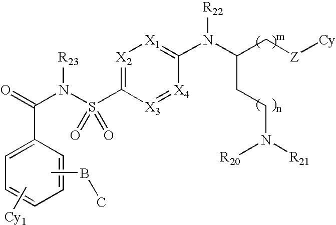 | |
| 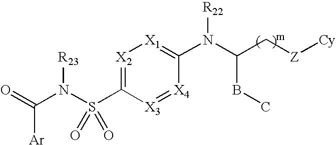 | (XIII) |

| Compound # | Structure |
|---|---|
| 1 | 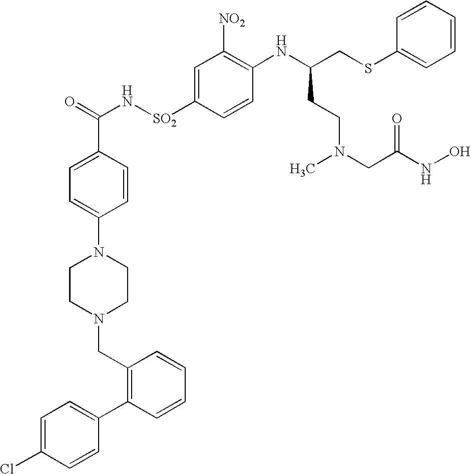 |
| 2 | 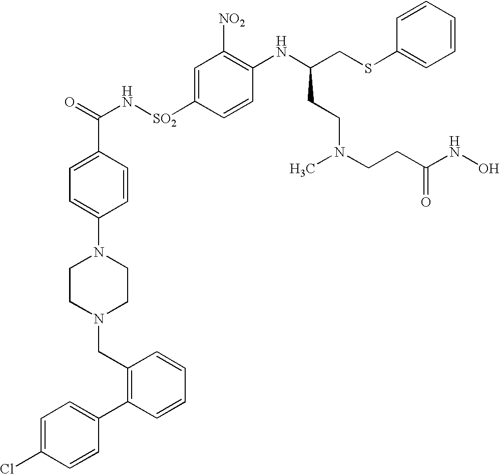 |
| 3 | 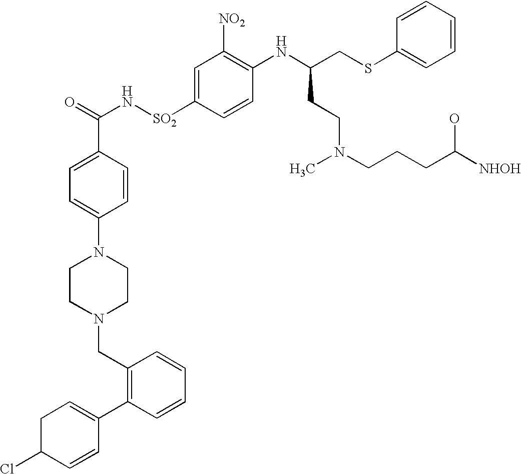 |
| 4 | 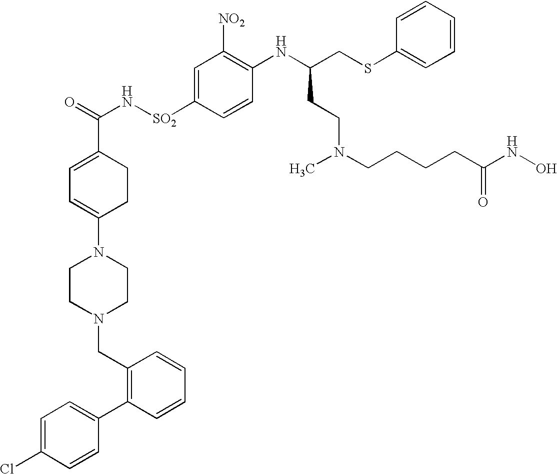 |
| 5 | 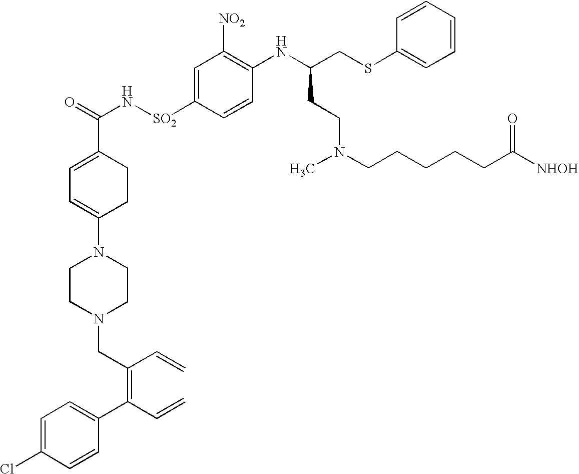 |
| 6 | 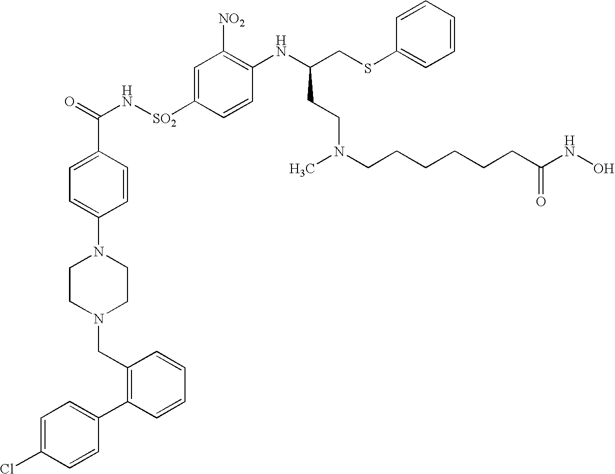 |
| 7 | 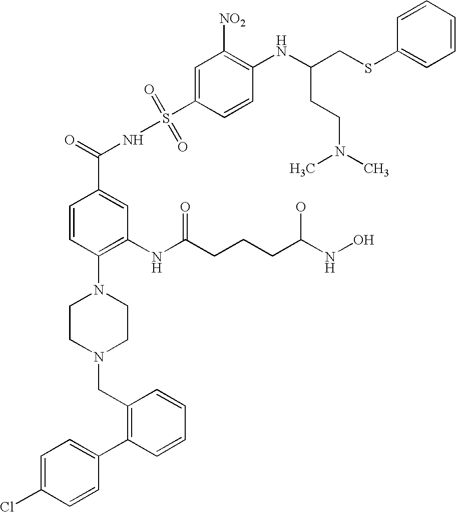 |
| 8 | 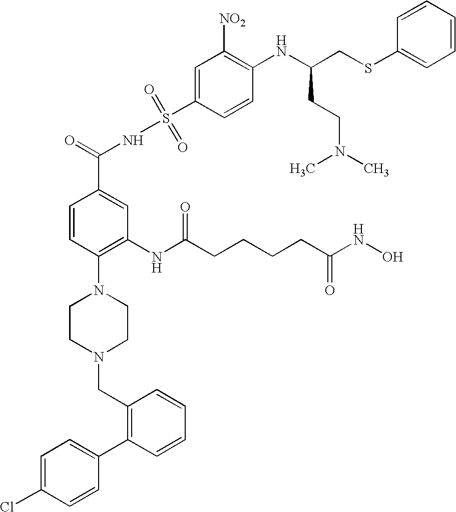 |
| 9 | 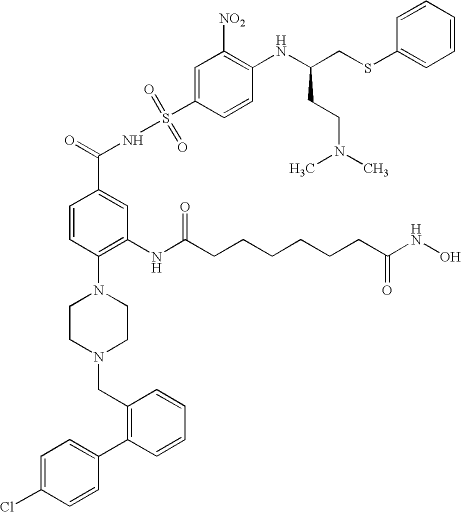 |
| 10 | 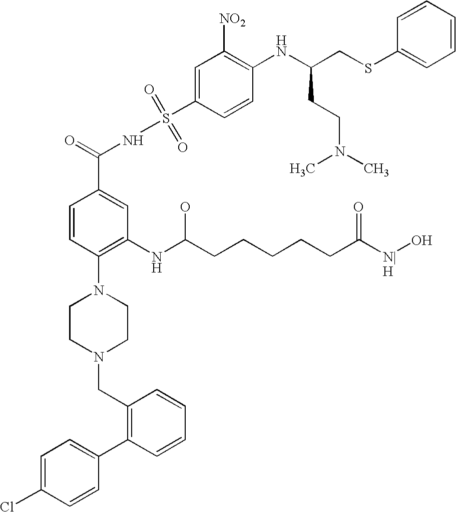 |

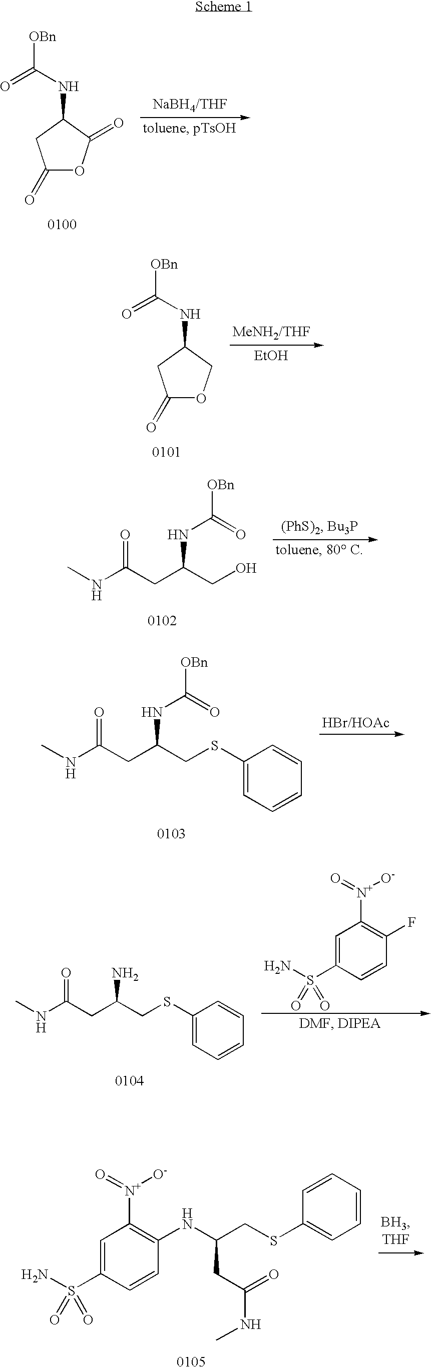

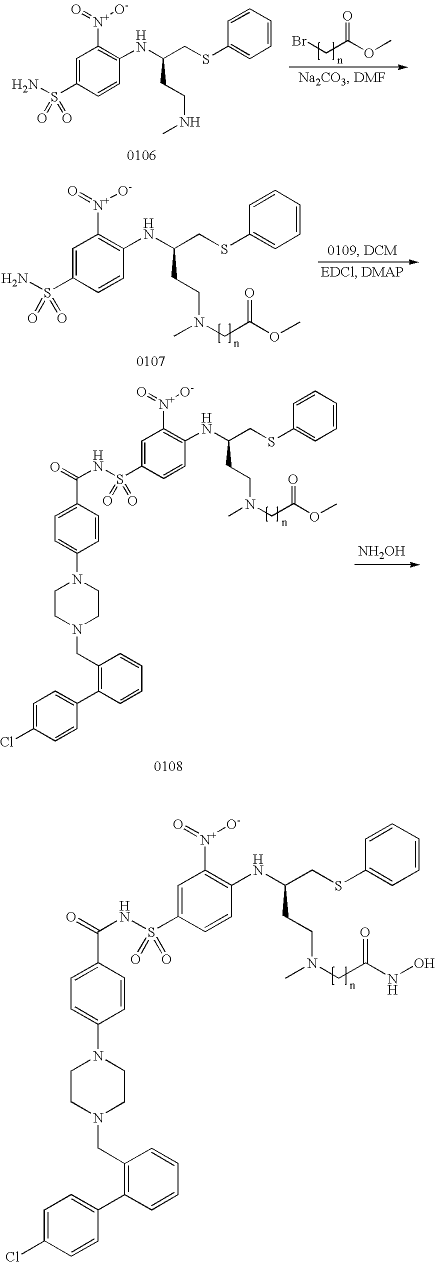

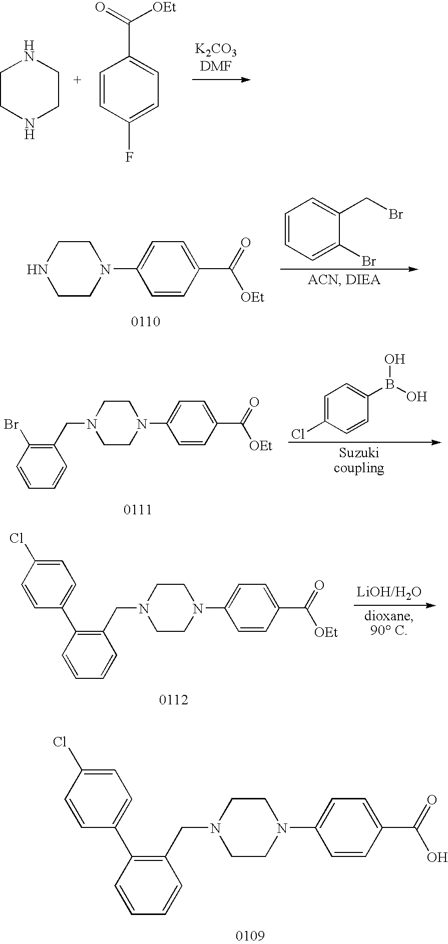

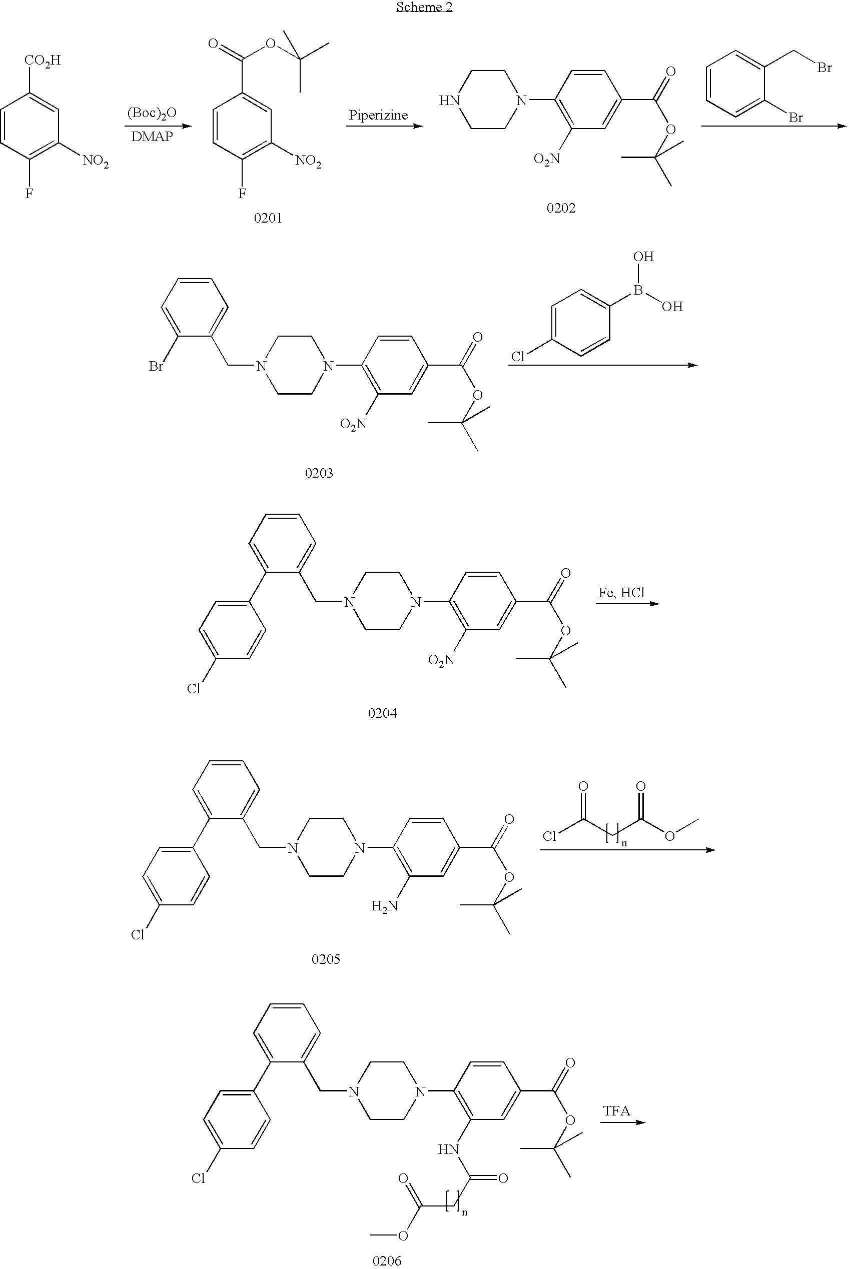

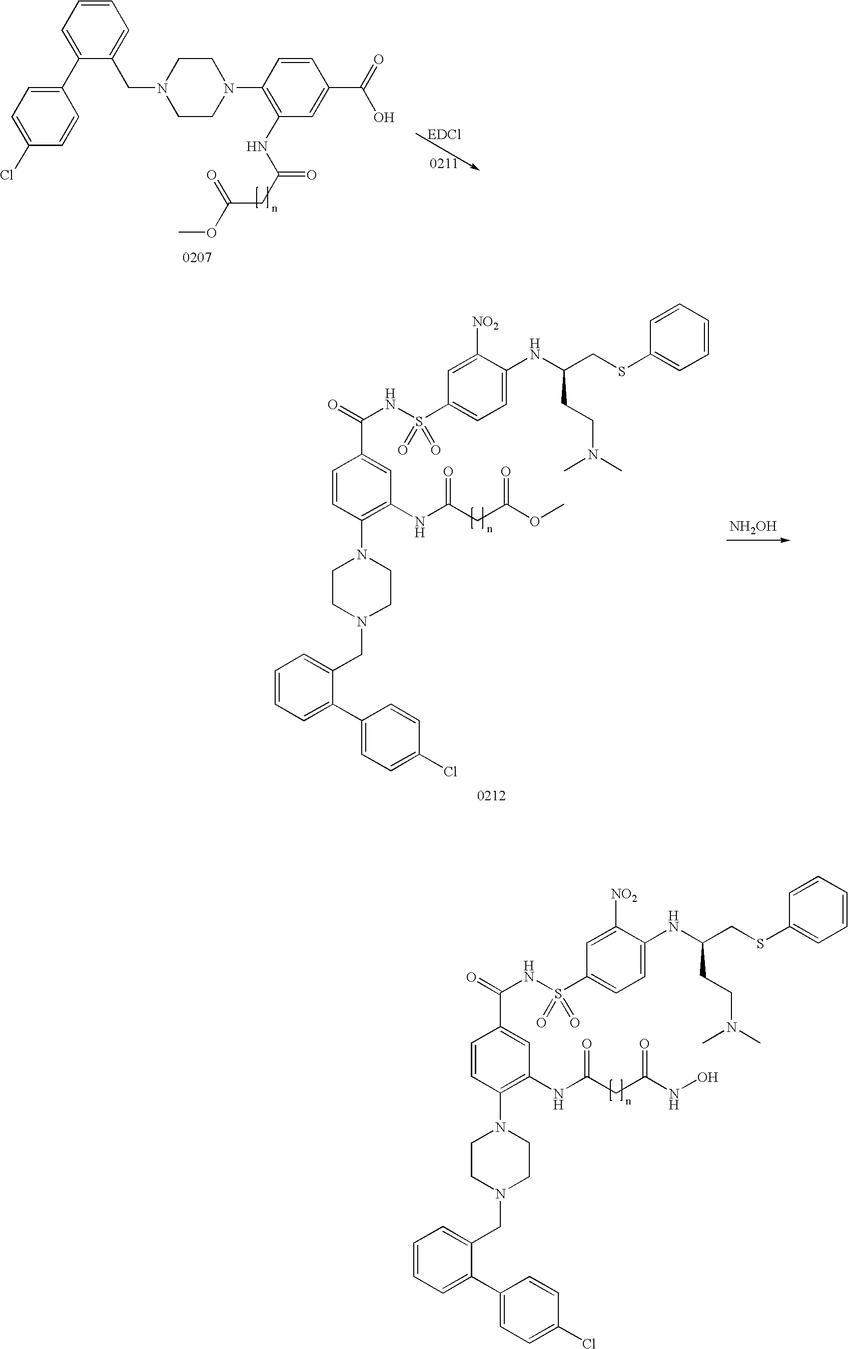

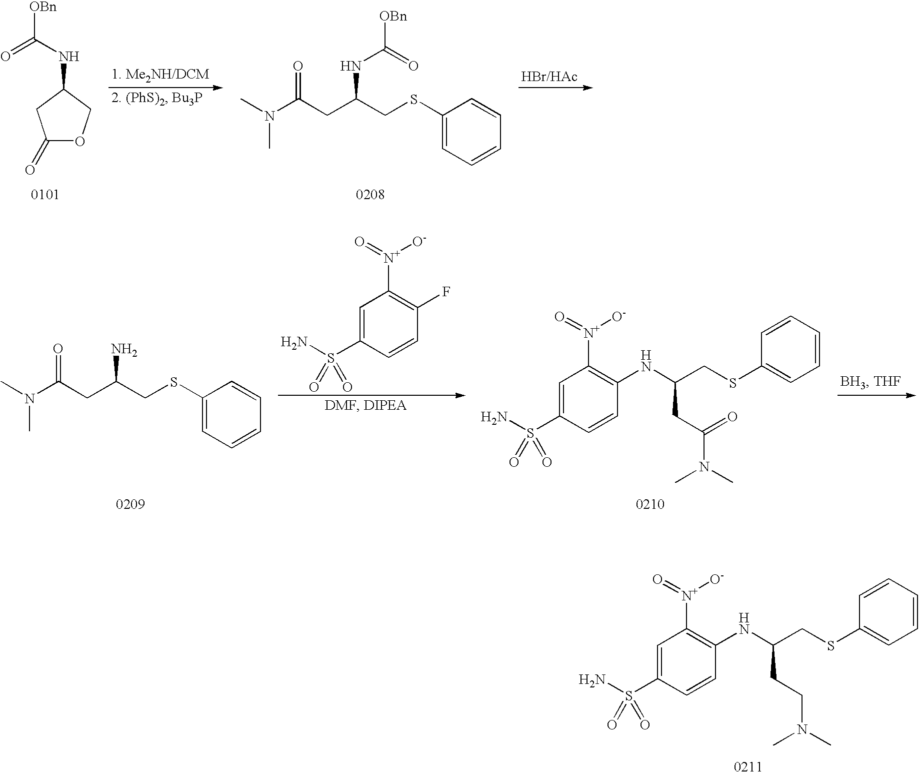

### Example 1

### Preparation of (R)-4-(4-((4′-chlorobiphenyl-2-yl)methyl)piperazin-1-yl)-N-(4-(4-((2-(hydroxyamino)-2-oxoethyl)(methyl)amino)-1-(phenylthio)-butan-2-ylamino)-3-nitrophenylsulfonyl)benzamide (Compound 1)

### Step 1a: (R)-Benzyl 5-oxo-tetrahydrofuran-3-ylcarbamate (Compound 0101)

To a stirred slurry of sodium borohydride (8.38 g, 0.223 mol) in THF (290 ml) at 0° C. was added a solution of 0100 (46 g, 0.185 mol) in THF (290 ml) over a period of 3 h. After stirring at room temperature for 1 h, the reaction mixture was carefully acidified to pH 2 with 6 N HCl and then concentrated to approximately one-fourth the volume under reduced pressure. The resulting solution was diluted with water and extracted with four portions of ether, and then the combined organic extracts were concentrated under reduced pressure to a heterogeneous residue. The yellow residue was taken up in toluene (200 ml), containing p-TsOH (200 mg), and then water was azeotropically removed by using a Dean-Stark apparatus. After the mixture refluxed for 5 h, the toluene was removed under reduced pressure to afford a viscous residue, which gave 0101 (37 g, 85%) as a white crystals upon triturated with ether. LCMS: 236 [M+1]⁻; ¹H NMR (DMSO-d₆): δ 2.39 (dd, 1H, J₁=3.6 Hz, J₂=18.0 Hz), 2.86 (dd, 1H, J₁=8.1 Hz, J₂=17.7 Hz), 4.11 (dd, 1H, J₁=3.6 Hz, J₂=9.3 Hz), 4.319 (m, 1H), 4.43 (dd, 1H, J₁=6.0 Hz, J₂=9.0 Hz), 5.05 (s, 2H), 7.365 (m, 5H), 7.88 (d, 1H, J=4.5 Hz).

### Step 1b: (R)-Benzyl 1-hydroxy-4-(methylamino)-4-oxobutan-2-ylcarbamate (Compound 0102)

0101 (5.04 g, 21.4 mmol) was added into a solution of methanamine (31.06 g, 1 mol) in ethanol (100 ml) and stirred for 15 m, during this period 0101 was dissolved gradually and then new solid appeared. The solvent was evaporated under reduced pressure to obtain 0102 (5.016 g, 88%) as a white solid which was used in the next step reaction without further purification. LCMS: 267 [M+1]⁺; ¹H NMR (DMSO-d₆): δ 2.18 (dd, 1H, J₁=8.4 Hz, J₂=14.1 Hz), 2.31 (dd, 1H, J₁=6.3 Hz, J₂=14.4 Hz), 2.54 (d, 3H, J=5.1 Hz), 3.33 (m, 1H), 3.82 (m, 1H), 4.703 (m, 1H), 5.00 (s, 2H), 6.98 (d, 1H, J=8.4 Hz), 7.35 (m, 5H), 7.68 (m, 1H).

### Step 1c: (R)-Benzyl 4-(methylamino)-4-oxo-1-(phenylthio)butan-2-ylcarbamate (Compound 0103)

A mixture of 0102 (5.02 g, 18.85 mmol), (PhS)₂ (8.23 g, 37.70 mmol) and PBu₃ (9.44 g, 40.98 mmol) in toluene (100 ml) was heated to 80° C. and stirred for 18 h. The mixture was cooled down and petroleum ether (500 ml) was added. The precipitate was filtered and washed with petroleum ether to obtain 0103 (5.45 g, 80.7%) as a white solid which was used in the next step reaction without further purification. LCMS: 359 [M+1]⁺. ¹H NMR (DMSO-d₆): δ 2.39 (m, 1H), 2.55 (d, 3H, J=3.9 Hz), 3.068 (m, 2H), 3.33 (m, 1H), 3.98 (m, 1H), 5.00 (s, 2H), 7.18 (m, 1H), 7.35 (m, 10H), 7.78 (m, 1H).

### Step 1d: (R)-3-Amino-N-methyl-4-(phenylthio)butanamide (Compound 0104)

0103 (5.4 g, 15.06 mmol) was dissolved in a mixture of acetic acid (100 ml) and 40% aqueous HBr solution (9.1 g) and stirred at 80° C. for 4 h. Water (100 ml) was added to the mixture after it's cooled down, extracted with methylene chloride (50 ml×2). The solution was adjusted pH=12 with 6N KOH, extracted with methylene chloride (100 ml×3), and the extract was dried with anhydrous sodium sulfate, evaporated under reduced pressure to obtain 0104 (2.5 g, 74%) as a colorless oil which was used in the next step reaction without further purification. LCMS: 225 [M+1]⁺. ¹H NMR (DMSO-d₆): δ 2.11 (dd, 1H, J₁=7.8 Hz, J₂=14.4 Hz), 2.31 (dd, 1H, J₁=5.1 Hz, J₂=15.0 Hz), 2.56 (d, 3H, J=4.5 Hz), 2.86 (dd, 1H, J₁=6.6 Hz, J₂=12.6 Hz), 3.03 (dd, 1H, J₁=5.1 Hz, J₂=12.6 Hz), 3.12 (m, 1H), 7.17 (m, 1H), 7.33 (m, 4H), 7.86 (m, 1H).

### Step 1e: (R)—N-Methyl-3-(2-nitro-4-sulfamoylphenylamino)-4-(phenylthio)butanamide (Compound 0105)

To the solution of 0104 (2.5 g, 11.14 mmol) in DMF (36 ml) were added 4-fluoro-3-nitrobenzenesulfonamide (2.7 g, 12.26 mmol) and DIPEA (1.9 ml). The mixture was stirred for 4 h. The solvent was evaporated under vacuum and the residue was purified by column chromatography on silica gel (methylene chloride/methanol=50:1) to yield 0105 (2.6 g, 55%) as a yellow solid. LCMS: 425 [M+1]⁺. ¹H NMR (DMSO-d₆): δ 2.55 (d, 3H, J=5.2 Hz), 2.63 (m, 2H), 3.34 (d, 2H, J=11.4 Hz), 4.38 (m, 1H), 7.07 (d, 1H, J=9.0 Hz), 7.23 (m, 7H), 7.72 (dd, 1H, J₁=2.1 Hz, J₂=9.0 Hz), 8.00 (d, 1H, J=4.5 Hz), 8.39 (d, 1H, J=2.1 Hz), 8.68 (d, 1H, J=9.6 Hz).

### Step 1f: (R)-4-(4-(Methylamino)-1-(phenylthio)butan-2-ylamino)-3-nitrobenzenesulfonamide (Compound 0106)

A mixture of 0105 (2 g, 4.7 mmol) and 1 M solution of BH₃ in THF (17 ml) was stirred for 16 h, and treated with methanol (5 ml) and concentrated HCl (2 ml). The resulting mixture was stirred at 80° C. for 2 h, cooled to room temperature, adjusted to pH=10 with 4 M Na₂CO₃. The solution was diluted with water (100 ml), extracted with methylene chloride (100 ml×2). The extracts was concentrated and purified by column chromatography on silica gel (methylene chloride/methanol=30:1) to yield 0106 (1.2 g, 62%) as a yellow solid. LCMS: 411 [M+1]. ¹H NMR (DMSO-d₆): δ 1.90 (m, 2H), 2.28 (s, 3H), 2.61 (t, 2H, J=6.6 Hz), 3.36 (m, 2H), 4.19 (m, 1H), 7.22 (m, 7H), 7.73 (dd, 1H, J₁=2.7 Hz, J₂=9.3 Hz), 8.39 (d, 1H, J=2.7 Hz), 8.52 (m, 1H).

### Step 1g: (R)-Ethyl 2-(methyl(3-(2-nitro-4-sulfamoylphenylamino)-4-(phenylthio)butyl)amino)acetate (Compound 0107-1)

A mixture of 0106 (313 mg, 0.762 mmol), ethyl 2-bromoacetate (127 mg, 0.762 mmol), Na₂CO₃ (323 mg, 3.05 mmol) in DMF (11 ml) was stirred at 50° C. for 16 h. DMF was evaporated under vacuum, and the residue was purified by column chromatography on silica gel (methylene chloride/methanol=30:1) to yield 0107-1 (323 mg, 85%) as a yellow solid. LCMS: 497 [M+1]⁺. ¹H NMR (DMSO-d₆): δ 1.15 (t, 3H, J=7.5 Hz), 1.83 (m, 1H), 1.95 (m, 1H), 2.24 (s, 3H), 2.54 (m, 2H), 3.21 (s, 2H), 3.38 (m, 2H), 4.04 (q, 2H, J=7.2 Hz), 4.16 (m, 1H), 7.22 (m, 8H), 7.70 (dd, 1H, J₁=2.7 Hz, J₂=9.3 Hz), 8.40 (d, 1H, J=2.7 Hz), 8.52 (d, 1H, J=8.7 Hz).

### Step 1h: (R)-Ethyl 2-((3-(4-(N-(4-(4-((4′-chlorobiphenyl-2-yl)methyl)piperazin-1-yl)-benzoyl)sulfamoyl)-2-nitrophenylamino)-4-(phenylthio)butyl)(methyl)amino)acetate (Compound 0108-1)

A mixture of 0107 (323 mg, 0.651 mmol), 0109 (291 mg, 0.716 mmol), EDCI (155 mg, 0.814 mmol) and DMAP (40 mg, 0.326 mmol) in anhydrous methylene chloride (4 ml) was stirred at room temperature for 16 h. The mixture was diluted with methylene chloride (50 ml), washed with brine (50 ml), dried over sodium sulfate, filtered and concentrated. The residue was purified by column chromatography on silica gel (methylene chloride/methanol=100:1) to yield 0108-1 (107 mg, 18.6%) as a yellow solid. LCMS: 443 [M/2+1]⁺.

### Step 1i: (R)-4-(4-((4′-Chlorobiphenyl-2-yl)methyl)piperazin-1-yl)-N-(4-(4-((2-(hydroxyamino)-2-oxoethyl)(methyl)amino)-1-(phenylthio)butan-2-ylamino)-3-nitro-phenylsulfonyl)benzamide (Compound 1)

To a stirred solution of hydroxylamine hydrochloride (4.67 g, 67 mmol) in methanol (24 ml) at 0° C. was added a solution of potassium hydroxide (5.61 g, 100 mmol) in methanol (14 ml). After addition, the mixture was stirred for 30 minutes at 0° C., and was allowed to stand at low temperature. The resulting precipitate was isolated to obtain the solution of free hydroxylamine in methanol.

A mixture of 0108-1 (107 mg, 0.121 mmol) and the NH₂OH solution (1.77 M, 3 ml) was stirred for 15 min at room temperature. The mixture was adjusted to pH 7.0 with acetic acid. The solution was concentrated to a small volume and water was added. The precipitate was filtered, and the collected solid was purified by prep-HPLC to afford compound 1 (47 mg, 44.6%) as a yellow solid. M.p.: 179˜201° C., LCMS: 872 [M+1]. ¹H NMR (DMSO-d₆): δ 1.97 (m, 2H), 2.34 (s, 3H), 2.41 (m, 4H), 2.66 (m, 2H), 3.12 (m, 2H), 3.22 (m, 4H), 3.35 (m, 2H), 3.41 (s, 2H), 4.20 (s, 1H), 6.87 (d, 2H, J=8.4 Hz), 7.19 (m, 7H), 7.49 (m, 7H), 7.75 (d, 2H, J=8.1 Hz), 7.83 (d, 1H, J=8.7 Hz), 8.42 (d, 1H, J=9.9 Hz), 8.50 (s, 1H), 8.95 (s, 1H), 10.61 (s, 1H).

### Step 1j: Ethyl 4-(piperazin-1-yl)benzoate (Compound 0110)

A mixture of piperazine (12.80 g, 0.15 mol), ethyl-4-fluorobenzoate (8.4 g, 0.05 mol) and K₂CO₃ (13.80 g, 0.10 mol) in DMSO (20 ml) was stirred at 120° C. for 6 h. The mixture was poured into water. The mixture was extracted with ethyl acetate and the organic layer was washed with water and brine, dried over Na₂SO₄, concentrated to give compound 0110 (12.40 g, 83%) as a white solid. LCMS: 235 [M+1]⁺.

### Step 1k: Ethyl 4-(4-(2-bromobenzyl)piperazin-1-yl)benzoate (Compound 0111)

A mixture of compound 0110 (3.778 g, 16.10 mmol), 2-bromobenzyl bromide (4.000 g, 16.10 mmol), and DIEA (3.4 ml) in acetonitrile (32 ml) was stirred at r.t. for 2 h. The precipitate was filtered to obtain compound 0111 (5.20 g, 80%) as a white solid. LCMS: 403 [M+1]⁺. ¹H NMR (DMSO-d₆): δ 1.29 (t, J=7.2 Hz, 3H), 2.55-2.59 (m, 4H), 3.29-3.34 (m, 4H), 3.60 (s, 2H), 4.25 (q, J=7.2 Hz, 2H), 6.97 (d, J=9 Hz, 2H), 7.19-7.25 (m, 1H), 7.38 (t, J=7.2 Hz, 1H), 7.52 (d, J=7.2 Hz, 1H), 7.61 (d, J=7.8 Hz, 1H), 7.77 (d, J=9 Hz, 2H).

### Step 1l: Ethyl 4-(4-((4′-chlorobiphenyl-2-yl)methyl)piperazin-1-yl)benzoate (Compound 0112)

A mixture of compound 0111 (6.915 g, 0.017 mol), 4-chlorophenylboronic acid (3.520 g, 0.023 mol), bis(triphenylpheosphine)palladium dichloride (240 mg, 0.340 mmol) and 2 M sodium carbonate (11.25 mL) in 7:3:2 DME/water/ethanol (100 mL) was stirred at 90° C. for 5 h. The mixture was cooled to room temperature and extracted with ethyl acetate. The extract was dried over anhydrous Na₂SO₄, filtered, and concentrated. The residue was purified by column chromatography on silica gel (ethyl acetate/petroether=2/5) to afford product (6.40 g, 86.7%). LCMS: 435 [M+1]⁻.

### Step 1m: 4-(4-((4′-Chlorobiphenyl-2-yl)methyl)piperazin-1-yl)benzoic acid (Compound 0109)

A mixture of compound 0112 (2.40 g, 5.53 mmol) and lithium hydroxide hydrate (0.70 g, 16.68 mmol) in a mixed solvents of dioxane (46 ml) and water (18 ml) was stirred at 95° C. overnight. The solvent was removed under reduced pressure and the residue was treated with 1 M HCl (15 mL), filtered to obtain compound 0109 (2.10 g, 93%) as a white solid. LCMS: 407 [M+1]⁺.

### Example 2

### Preparation of (R)-4-(4-((4′-chlorobiphenyl-2-yl)methyl)piperazin-1-yl)-N-(4-(4-((3-(hydroxyamino)-3-oxopropyl)(methyl)amino)-1-(phenylthio)-butan-2-ylamino)-3-nitrophenylsulfonyl)benzamide (Compound 2)

### Step 2a: (R)-Methyl 3-(methyl(3-(2-nitro-4-sulfamoylphenylamino)-4-(phenylthio)-butyl)amino)propanoate (Compound 0107-2)

The title compound 0107-2 was prepared as a yellow solid (247 mg, 45.0%) from compound 0106 (454 mg, 1.11 mmol), methyl 3-bromopropanoate (185 mg, 1.11 mmol), Na₂CO₃ (469 mg, 4.44 mmol) in DMF (15 ml) using a procedure similar to that described for compound 0107-1 (Example 1): LCMS: 497 [M+1]⁺.

### Step 2b: (R)-Methyl 3-((3-(4-(N-(4-(4-((4′-chlorobiphenyl-2-yl)methyl)piperazin-1-yl)benzoyl)sulfamoyl)-2-nitrophenylamino)-4-(phenylthio)butyl)(methyl)amino)propanoate (Compound 0108-2)

The title compound 0108-2 was prepared as a yellow solid (231 mg, 52.5%) from compound 0107-2 (247 mg, 0.497 mmol), 0109 (222 mg, 0.547 mmol), EDCI (119 mg, 0.621 mmol) and DMAP (31 mg, 0.249 mmol) using a procedure similar to that described for compound 0107-1 (Example 1): LCMS: 443 [M/2+1]⁺.

### Step 2c: (R)-4-(4-((4′-Chlorobiphenyl-2-yl)methyl)piperazin-1-yl)-N-(4-(4-((3-(hydroxyamino)-3-oxopropyl)(methyl)amino)-1-(phenylthio)butan-2-ylamino)-3-nitrophenylsulfonyl)benzamide (Compound 2)

The title compound 2 was prepared as a yellow solid (53 mg, 38.4%) using a procedure similar to that described for compound 1 (Example 1): M.p.: 130˜138° C. LCMS: 886 [M+1]. ¹H NMR (DMSO-d₆): δ 2.05 (m, 2H), 2.29 (s, 3H), 2.40 (m, 6H), 2.98 (m, 4H), 3.17 (m, 6H), 3.39 (s, 2H), 4.20 (s, 1H), 6.83 (d, 2H, J=8.4 Hz), 6.99 (d, 1H, J=9.3 Hz), 7.41 (m, 13H), 7.73 (d, 2H, J=9.0 Hz), 7.82 (d, 1H, J=9 Hz), 8.28 (d, 1H, J=8.1 Hz), 8.47 (s, 1H), 8.88 (s, 1H), 10.56 (s, 1H).

### Example 3

### Preparation of (R)-4-(4-((4′-chlorobiphenyl-2-yl)methyl)-piperazin-1-yl)-N-(4-(4-((4-(hydroxyamino)-4-oxobutyl)(methyl)amino)-1-(phenylthio)butan-2-ylamino)-3-nitrophenylsulfonyl)benzamide (Compound 3)

### Step 3a: (R)-Ethyl 4-(methyl(3-(2-nitro-4-sulfamoylphenylamino)-4-(phenylthio)-butyl)amino)butanoate (Compound 0107-3)

The title compound 0107-3 was prepared as a yellow solid (198 mg, 52%) from compound 0106 (300 mg, 0.731 mmol), ethyl 4-bromobutanoate (143 mg, 0.731 mmol), Na₂CO₃ (310 mg, 2.924 mmol) in DMF (10 ml) using a procedure similar to that described for compound 0107-1 (Example 1): LCMS: 525 [M+1]⁺. ¹H NMR (DMSO-d₆): δ 1.15 (t, 3H, J=6.9 Hz), 1.60 (m, 2H), 1.83 (m, 1H), 1.95 (m, 1H), 2.09 (s, 3H), 2.22 (m, 5H), 3.36 (m, 2H), 4.01 (q, 2H, J=6.9 Hz), 4.12 (m, 1H), 7.06 (d, 1H, J=9.0 Hz), 7.27 (m, 7H), 7.72 (dd, 1H, J₁=2.1, J₂=9.0), 8.40 (d, 1H, J=2.1 Hz), 8.50 (d, 1H, J=9.3 Hz).

### Step 3b: (R)-Ethyl 4-((3-(4-(N-(4-(4-((4′-chlorobiphenyl-2-yl)methyl)piperazin-1-yl)-benzoyl)sulfamoyl)-2-nitrophenylamino)-4-(phenylthio)butyl)(methyl)amino)-butanoate (Compound 0108-3)

The title compound 0108-3 was prepared as a yellow solid (150 mg, 43.6%) from compound 0107-3 (198 mg, 0.377 mmol), 0109 (230 mg, 0.566 mmol), EDCI (108 mg, 0.566 mmol) and DMAP (23 mg, 0.189 mmol) using a procedure similar to that described for compound 0108-1 (Example 1): LCMS: 457 [M/2+1]⁺. ¹H NMR (DMSO-d₆): δ 1.16 (t, 3H, J=7.2 Hz), 1.76 (m, 2H), 2.06 (m, 2H), 2.32 (t, 2H, J=7.5 Hz), 2.40 (m, 4H), 2.55 (m, 3H), 2.80 (m, 4H), 3.16 (m, 4H), 3.24 (m, 2H), 3.39 (s, 2H), 4.04 (q, 2H, J=6.9 Hz), 4.12 (m, 1H), 6.82 (d, 2H, J=9.0 Hz), 6.97 (d, 1H, J=9.6 Hz), 7.47 (m, 14H), 7.73 (d, 2H, J=8.7 Hz), 7.82 (d, 1H, J=9.6 Hz), 8.24 (d, 1H, J=8.4 Hz), 8.48 (s, 1H).

### Step 3c: (R)-4-(4-((4′-Chlorobiphenyl-2-yl)methyl)piperazin-1-yl)-N-(4-(4-((4-(hydroxylamino)-4-oxobutyl)(methyl)amino)-1-(phenylthio)butan-2-ylamino)-3-nitro-phenylsulfonyl)benzamide (Compound 3)

The title compound 3 was prepared as a yellow solid (19 mg, 12.8%) using a procedure similar to that described for compound 1 (Example 1): LCMS: 900 [M+1]⁺. ¹H NMR (DMSO-d₆): δ 1.64 (m, 2H), 1.93 (m, 4H), 2.67 (m, 2H), 2.40 (m, 6H), 3.13 (m, 4H), 3.38 (s, 2H), 4.06 (s, 1H), 6.79 (d, 2H, J=9.3 Hz), 6.86 (d, 1H, J=9.6 Hz), 7.32 (m, 14H), 7.73 (m, 3H), 8.32 (m, 1H), 8.43 (s, 1H), 8.70 (m, 1H), 10.42 (m, 1H).

### Example 4

### Preparation of (R)-4-(4-((4′-chlorobiphenyl-2-yl)methyl)-piperazin-1-yl)-N-(4-(4-((5-(hydroxyamino)-5-oxopentyl)(methyl)amino)-1-(phenylthio)butan-2-ylamino)-3-nitrophenylsulfonyl)benzamide (Compound 4)

### Step 4a: (R)-Methyl 5-(methyl(3-(2-nitro-4-sulfamoylphenylamino)-4-(phenylthio)-butyl)amino)pentanoate (Compound 0107-4)

The title compound 0107-4 was prepared as a yellow solid (194 mg, 51%) from compound 0106 (300 mg, 0.731 mmol), methyl 5-bromopentanoate (143 mg, 0.731 mmol), Na₂CO₃ (310 mg, 2.924 mmol) in DMF (10 ml) using a procedure similar to that described for compound 0107-1 (Example 1): LCMS: 525 [M+1]⁺. ¹H NMR (DMSO-d₆): δ 1.36 (m, 2H), 1.44 (m, 2H), 1.83 (m, 1H), 1.95 (m, 1H), 2.08 (s, 3H), 2.24 (m, 5H), 2.44 (m, 1H), 3.35 (m, 2H), 3.56 (s, 3H), 4.12 (m, 1H), 7.06 (d, 1H, J=9.3 Hz), 7.32 (m, 8H), 7.71 (dd, 1H, J₁=2.4, J₂=9.0), 8.41 (d, 1H, J=1.5 Hz), 8.51 (d, 1H, J=8.4 Hz).

### Step 4b: (R)-Methyl 5-((3-(4-(N-(4-(4-((4′-chlorobiphenyl-2-yl)methyl)piperazin-1-yl)benzoyl)sulfamoyl)-2-nitrophenylamino)-4-(phenylthio)butyl)(methyl)amino)pentanoate (Compound 0108-4)

The title compound 0108-4 was prepared as a yellow solid (167 mg, 49.4%) from compound 0107-4 (194 mg, 0.370 mmol), 0109 (225 mg, 0.555 mmol), EDCI (106 mg, 0.555 mmol) and DMAP (230 mg, 0.189 mmol) using a procedure similar to that described for compound 0108-1 (Example 1): LCMS: 457 [M/2+1]⁺. ¹H NMR (DMSO-d₆): δ 1.45 (m, 3H), 2.32 (m, 3H), 2.40 (m, 4H), 2.60 (m, 2H), 2.72 (m, 2H), 3.07 (m, 3H), 3.14 (m, 4H), 3.25 (m, 2H), 3.56 (s, 2H), 4.06 (m, 1H), 6.79 (d, 2H, J=7.5 Hz), 6.90 (m, 1H), 7.26 (m, 6H), 7.49 (m, 5H), 7.75 (m, 2H), 8.16 (d, 1H, J=7.2 Hz), 8.28 (d, 1H, J=8.6 Hz), 8.44 (d, 1H, J=2.1 Hz).

### Step 4c: (R)-4-(4-((4′-Chlorobiphenyl-2-yl)methyl)piperazin-1-yl)-N-(4-(4-((5-(hydroxyamino)-5-oxopentyl)(methyl)amino)-1-(phenylthio)butan-2-ylamino)-3-nitrophenylsulfonyl)benzamide (Compound 4)

The title compound 4 was prepared as a yellow solid (50 mg, 30%) using a procedure similar to that described for compound 1 (Example 1): M.p.: 126˜130° C., LCMS: 914 [M+1]⁺. ¹H NMR (DMSO-d₆) δ 1.47 (m, 4H), 1.95 (m, 2H), 2.10 (m, 2H), 2.40 (m, 4H), 2.64 (m, 3H), 3.15 (m, 4H), 3.39 (s, 2H), 4.10 (m, 1H), 6.80 (d, 2H, J=8.7 Hz), 6.93 (d, 1H, J=9.0 Hz), 7.24 (m, 7H), 7.48 (m, 6H), 7.72 (d, 2H, J=8.7 Hz), 7.81 (d, 1H, J=9.6 Hz), 8.21 (m, 1H), 8.46 (d, 1H, J=2.1 Hz), 8.70 (s, 1H), 10.38 (s, 1H).

### Example 5

### Preparation of (R)-4-(4-((4′-chlorobiphenyl-2-yl)methyl)-piperazin-1-yl)-N-(4-(4-((6-(hydroxyamino)-6-oxohexyl)(methyl)amino)-1-(phenylthio)butan-2-ylamino)-3-nitrophenylsulfonyl)benzamide (Compound 5)

### Step 5a: (R)-Ethyl 6-(methyl(3-(2-nitro-4-sulfamoylphenylamino)-4-(phenylthio)-butyl)amino)hexanoate (Compound 0107-5)

The title compound 0107-5 was prepared as a yellow solid (220 mg, 54.5%) from compound 0106 (300 mg, 0.731 mmol), ethyl 6-bromohexanoate (163 mg, 0.731 mmol), Na₂CO₃ (310 mg, 2.924 mmol) in DMF (10 ml) using a procedure similar to that described for compound 0107-1 (Example 1): LCMS: 553 [M+1]⁺. ¹H NMR (DMSO-d₆): δ 1.17 (m, 5H), 1.31 (m, 2H), 1.45 (m, 2H), 1.81 (m, 1H), 1.96 (m, 1H), 2.08 (s, 3H), 2.20 (m, 4H), 2.43 (m, 2H), 3.33 (m, 2H), 4.03 (q, 2H, J=6.9 Hz), 4.12 (m, 1H), 7.04 (d, 1H, J=9.6 Hz), 7.30 (m, 7H), 7.69 (dd, 1H, J₁=2.1, J₂=9.0), 8.39 (d, 1H, J=2.1 Hz), 8.51 (d, 1H, J=8.7 Hz).

### Step 5b: (R)-Ethyl 6-((3-(4-(N-(4-(4-((4′-chlorobiphenyl-2-yl)methyl)piperazin-1-yl)benzoyl)sulfamoyl)-2-nitrophenylamino)-4-(phenylthio)butyl)(methyl)amino)-hexanoate (Compound 0108-5)

The title compound 0108-5 was prepared as a yellow solid (165 mg, 48%) from compound 0107-5 (202 mg, 0.365 mmol), 0109 (163 mg, 0.402 mmol), EDCI (87 mg, 0.457 mmol) and DMAP (190 mg, 0.152 mmol) in anhydrous methylene chloride (2.6 ml) using a procedure similar to that described for compound 0108-1 (Example 1): LCMS: 471 [M/2−1]⁺. ¹H NMR (DMSO-d₆): δ 1.21 (m, 5H), 1.51 (m, 4H), 2.09 (m, 2H), 2.26 (t, 2H, J=6.6 Hz), 2.28 (m, 4H), 2.60 (m, 3H), 3.15 (m, 4H), 3.39 (s, 2H), 4.06 (m, 3H), 6.80 (d, 2H, J=9.3 Hz), 6.93 (d, 1H, J=9.6 Hz), 7.48 (m, 13H), 7.72 (d, 2H, J=9.3 Hz), 7.82 (d, 1H, J=9.6 Hz), 8.18 (m, 1H), 8.47 (s, 1H).

### Step 5c: (R)-4-(4-((4′-Chlorobiphenyl-2-yl)methyl)piperazin-1-yl)-N-(4-(4-((6-(hydroxyamino)-6-oxohexyl)(methyl)amino)-1-(phenylthio)butan-2-ylamino)-3-nitro-phenylsulfonyl)benzamide (Compound 5)

The title compound 5 was prepared as a yellow solid (18 mg, 28%) using a procedure similar to that described for compound 1 (Example 1): LCMS: 928 [M+1]⁺. ¹H NMR (DMSO-d₆): δ 1.20 (m, 2H), 1.48 (m, 4H), 1.93 (t, 2H, J=7.5 Hz), 2.09 (m, 2H), 2.40 (m, 4H), 2.66 (s, 3H), 2.91 (m, 2H), 3.13 (m, 6H), 3.41 (m, 4H), 4.13 (m, 1H), 6.80 (d, 2H, J=9.3 Hz), 6.93 (d, 1H, J=9.3 Hz), 7.26 (m, 8H), 7.48 (m, 6H), 7.72 (d, 2H, J=8.7 Hz), 7.82 (dd, 1H, J₁=1.8 Hz, J₂=9.0 Hz), 8.19 (m, 1H), 8.46 (d, 1H, J=2.4 Hz), 8.68 (s, 1H), 10.35 (s, 1H).

### Example 6

### Preparation of (R)-4-(4-((4′-chlorobiphenyl-2-yl)methyl)-piperazin-1-yl)-N-(4-(4-((7-(hydroxyamino)-7-oxoheptyl)(methyl)amino)-1-(phenylthio)butan-2-ylamino)-3-nitrophenylsulfonyl)benzamide (Compound 6)

### Step 6a: (R)-Ethyl 7-(methyl(3-(2-nitro-4-sulfamoylphenylamino)-4-(phenylthio)-butyl)amino)heptanoate (Compound 0107-6)

The title compound 0107-6 was prepared as a yellow solid (224 mg, 54%) from compound 0106 (300 mg, 0.731 mmol), ethyl 7-bromoheptanoate (173 mg, 0.731 mmol), Na₂CO₃ (310 mg, 2.924 mmol) in DMF (10 ml) using a procedure similar to that described for compound 0107-1 (Example 1): LCMS: 567 [M+1]⁺. ¹H NMR (DMSO-d₆): δ 1.16 (m, 7H), 1.30 (m, 2H), 1.45 (m, 2H), 1.81 (m, 1H), 1.96 (m, 1H), 2.09 (s, 3H), 2.22 (m, 4H), 2.46 (m, 2H), 3.33 (m, 2H), 4.03 (q, 2H, J=6.9 Hz), 4.12 (m, 1H), 7.05 (d, 1H, J=9.6 Hz), 7.33 (m, 7H), 7.70 (m, 1H), 8.40 (s, 1H), 8.54 (d, 1H, J=8.1 Hz).

### Step 6b: (R)-Ethyl 7-((3-(4-(N-(4-(4-((4′-chlorobiphenyl-2-yl)methyl)piperazin-1-yl)benzoyl)sulfamoyl)-2-nitrophenylamino)-4-(phenylthio)butyl)(methyl)amino)-heptanoate (Compound 0108-6)

The title compound 0108-6 was prepared as a yellow solid (190 ml, 41%) from compound 0107-6 (220 mg, 0.395 mmol), 0109 (241 mg, 0.593 mmol), EDCI (94 mg, 0.494 mmol) and DMAP (240 mg, 0.196 mmol) using a procedure similar to that described for compound 0108-1 (Example 1): LCMS: 478 [M/2+1]⁺. ¹H NMR (DMSO-d₆): δ 1.16 (t, 3H, J=7.5 Hz), 1.25 (m, 5H), 1.49 (m, 3H), 2.10 (m, 2H), 2.25 (t, 2H, J=7.2 Hz), 2.40 (m, 4H), 2.60 (m, 3H), 2.85 (m, 2H), 3.15 (m, 4H), 3.24 (m, 2H), 3.39 (s, 2H), 4.03 (q, 2H, J=7.2 Hz), 4.08 (m, 1H), 6.80 (d, 1H, J=9.3 Hz), 6.93 (m, 1H), 7.38 (m, 13H), 7.76 (m, 4H), 8.19 (d, 1H, J=7.5 Hz), 8.46 (d, 1H, J=1.5 Hz).

### Step 6c: (R)-4-(4-((4′-chlorobiphenyl-2-yl)methyl)piperazin-1-yl)-N-(4-(4-((7-(hydroxyamino)-7-oxoheptyl)(methyl)amino)-1-(phenylthio)butan-2-ylamino)-3-nitrophenylsulfonyl)benzamide (compound 6)

The title compound 6 was prepared as a yellow solid (60 mg, 33%) using a procedure similar to that described for compound 1 (Example 1): M.p.: 125˜130° C. LCMS: 942 [M+1]⁺. ¹H NMR (DMSO-d₆): δ 1.21 (m, 4H), 1.46 (m, 4H), 1.92 (t, 2H, J=5.2 Hz), 2.10 (m, 2H), 2.40 (m, 4H), 2.58 (s, 3H), 2.85 (m, 4H), 3.14 (m, 4H), 3.35 (m, 2H), 3.39 (s, 2H), 4.09 (m, 1H), 6.80 (d, 2H, J=8.7 Hz), 6.93 (d, 1H, J=9.3 Hz), 7.26 (m, 7H), 7.48 (m, 6H), 7.73 (d, 2H, J=9.0 Hz), 7.81 (dd, 1H, J₁=1.8 Hz, J₂=9.0 Hz), 8.21 (m, 1H), 8.46 (d, 1H, J=1.8 Hz), 8.67 (s, 1H), 10.34 (s, 1H).

### Example 7

### Preparation of (R)—N¹-(2-(4-((4′-chlorobiphenyl-2-yl)methyl)-piperazin-1-yl)-5-(4-(4-(dimethylamino)-1-(phenylthio)butan-2-ylamino)-3-nitrophenylsulfonylcarbamoyl)phenyl)-N⁵-hydroxyglutaramide (Compound 7)

### Step 7a: tert-Butyl 4-fluoro-3-nitrobenzoate (Compound 0201)

To a solution of 4-fluoro-3-nitro benzoic acid (370 mg, 2 mmol) in 10 mL of t-BuOH were added (Boc)₂O (872 mg, 4 mmol) and DMAP (24 mg, 0.2 mmol). The solution was stirred for 24 hours. The solvent was evaporated. The residue was dissolved in ethyl acetate and washed with 1N HCl. The separated organic phase was evaporated. The residue was subjected to a flash column chromatography on silica gel eluting with 12.5% EtOAc/Petroleum ether to give compound 0201 (240 mg, 49.8%). ¹H NMR (DMSO-d₆): δ 1.55 (s, 9H), 7.69 (m, 1H), 8.26 (m, 1H), 8.48 (m, 1H).

### Step 7b: tert-Butyl 3-nitro-4-(piperazin-1-yl)benzoate (Compound 0202)

A mixture of piperazine (451 mg, 5.2 mmol), tert-butyl 4-fluoro-3-nitro-benzoate (211 mg, 0.9 mmol) and K₂CO₃ (234 mg, 1.7 mmol) in DMF (10 ml) was stirred at 120° C. for 6 hours. The mixture was poured into water, and extracted with ethyl acetate. The organic phase was washed with water (100 ml), concentrated in vacuo. The residue was purified with flash column chromatography on silica gel eluting with 25% ethyl acetate/petroleum ether to provide 0202 (190 mg, 70.7%). LC-MS: 308 [M+1]. ¹H NMR (CDCl₃): δ 1.58 (s, 9H), 1.84 (s, 1H), 3.01 (m, 4H), 3.12 (m, 4H), 7.03 (d, J=6.0 Hz, 1H), 8.02 (dd, J=2.1, 6.0 Hz, 1H), 8.33 (d, J=2.1 Hz, 1H).

### Step 7c: tert-Butyl 4-(4-(2-bromobenzyl)piperazin-1-yl)-3-nitrobenzoate (Compound 0203)

A mixture of 0202 (262 mg, 0.85 mmol), 2-bromobenzyl bromide (161 mg, 0.65 mmol), and DIEA (149 mg, 1.3 mmol) in acetonitrile (6 ml) was stirred at 25° C. for 2 hours and filtered. The solid was subjected to column chromatography on silica gel eluting with ethyl acetate to give 0203 (320 mg, 78.7%). LC-MS: 476 [M+1]⁺. ¹H NMR (CDCl₃): δ 1.57 (s, 9H), 2.67 (t, J=4.8 Hz, 4H), 3.18 (t, J=4.8 Hz, 4H), 3.66 (s, 2H), 7.03 (d, J=8.4 Hz, 1H), 7.12 (m, 1H), 7.28 (m, 1H), 7.45 (m, 1H), 7.56 (m, 1H), 8.00 (dd, J=2.1, 6.0 Hz, 1H), 8.33 (d, J=2.1 Hz, 1H).

### Step 7d: tert-Butyl 4-(4-((4′-chlorobiphenyl-2-yl)methyl)piperazin-1-yl)-3-nitrobenzoate (Compound 0204)

A mixture of 0203 (160 mg, 0.3 mmol), 4-chlorophenylboronic acid (51 mg, 0.3 mmol), bis(triphenylphosphine)palladium dichloride (7 mg, 0.01 mmol) and 2M sodium carbonate (0.15 mL) in a mixed solvent of DME/water/ethanol (7/3/2, 5 mL) was stirred at 90° C. overnight and extracted with ethyl acetate. The extract was dried (MgSO₄), filtered, and concentrated. The residue was purified by flash column chromotography on silica gel eluting with 5%-40% ethyl acetate/petroleum ether to give 0204 (90 mg, 52.7%). LC-MS: 508 [M+1]⁺. ¹H NMR (CDCl₃): δ 1.57 (s, 9H), 2.50 (t, J=4.8 Hz, 4H), 3.10 (t, J=4.8 Hz, 4H), 3.43 (s, 2H), 7.00 (d, J=8.7 Hz, 1H), 7.25 (m, 1H), 7.32 (m, 2H), 7.35 (m, 4H), 7.49 (m, 1H), 8.00 (m, 1H), 8.32 (d, J=2.1 Hz, 1H).

### Step 7e: tert-Butyl 3-amino-4-(4-((4′-chlorobiphenyl-2-yl)methyl)-piperazin-1-yl)benzoate (Compound 0205)

Compound 4705 (13.4 g, 26 mmol) was dissolved in methanol (300 ml), and the solution was heated to 60° C. To the solution Fe powder (14.6 g, 260 mmol) and diluted HCl (2.3 g in 10 mL of CH₃OH) were added. The mixture was stirred for 4 hours, and then the solvent was removed under vacuo. The residue was purified by flash column chromatography on silica gel eluting with 10% MeOH/CH₂Cl₂ to give 0205 (6.0 g, 50.3%). LC-MS: 478 [M+1]⁺. ¹H NMR (CDCl₃): δ 1.55 (s, 9H), 2.52 (br, 4H), 2.91 (br, 4H), 3.39 (s, 2H), 3.91 (s, 2H), 6.95 (m, 1H), 7.24 (m, 1H), 7.33 (m, 4H), 7.38 (m, 4H), 7.52 (m, 1H).

### Step 7f: tert-butyl4-(4-((4′-chlorobiphenyl-2-yl)methyl)piperazin-1-yl)-3-(5-methoxy-5-oxopentanamido)benzoate (Compound 0206-7)

To a mixture of 0205 (1 g, 2 mmol) and DIEA (516 mg, 4 mmol) in CH₂Cl₂ (20 ml) was added methyl 5-chloro-5-oxopentanoate (343 mg, 2 mmol) at 0° C. The mixture was then warmed to room temperature and stirred for one hour. The solvent was removed in vacuo, and the residue was subjected to column chromatography on silica gel eluting with 25% EtOAc/petroleum ether to provide 0206-7 (1.03 g, 81.1%). LC-MS: 606 [M+1]. ¹H NMR (CDCl₃): δ 1.57 (s, 9H), 2.04 (m, 2H), 2.45 (m, 4H), 2.54 (br, 4H), 2.84 (t, J=4.5 Hz, 4H), 3.46 (S, 2H), 3.66 (s, 3H), 7.11 (m, 1H), 7.23 (m, 1H), 7.38 (m, 6H), 7.57 (m, 1H), 7.71 (m, 1H), 8.23 (s, 1H), 8.87 (s, 1H).

### Step 7g: 4-(4-((4′-chlorobiphenyl-2-yl)methyl)piperazin-1-yl)-3-(5-methoxy-5-oxopentanamido)benzoic acid (Compound 0207-7)

To a solution of 0206-7 (900 mg, 1.5 mmol) in CH₂Cl₂ (10 ml) was added trifluoroacetic acid (1 ml). The resulting mixture was stirred overnight at room temperature. The solvent was removed in vacuo to give 0207-7 (760 mg, 93.2%). The compound was used in the next step reaction without further purification. LC-MS: 550 [M+1]⁻.

### Step 7h: (R)-Benzyl 4-(dimethylamino)-4-oxo-1-(phenylthio)butan-2-ylcarbamate (Compound 0208)

Compound 0101 (24 g, 0.1 mol) was added to the solution of Me₂NH (45 g, 1 mol) in CH₂Cl₂ (500 ml). The mixture was stirred overnight. The solid was collected by filtration. Toluene (500 mL) was added to dissolve the solid, followed by (PhS)₂ (32.7 g, 0.15 mol) and Bu₃P (40 g, 0.2 mol). The mixture was heated to 80° C. and stirred for 18 h. The solvent was removed in vacuo. The residue was subjected to flash column chromatography on silica gel eluting with 50% EtOAc/petroleum ether to provide 0208 (13.4 g, 35.3%). LC-MS: 373 [M+1]⁺. ¹H NMR (CDCl₃): δ 2.46 (m, 1H), 2.82 (s, 3H), 2.84 (s, 3H), 2.88 (m, 1H), 3.20 (m, 1H), 3.33 (m, 1H), 4.13 (m, 1H), 5.07 (s, 2H), 6.30 (d, J=9.0 Hz, 1H), 7.15 (m, 1H), 7.32 (m, 9H).

### Step 7i: (R)-3-Amino-N,N-dimethyl-4-(phenylthio)butanamide (Compound 0209)

To a solution of 0208 (664 mg, 1.8 mmol) in 12 ml of HOAc was added HBr (432 mg, 40% water solution) at room temperature. The mixture was heated to 80° C. and stirred for 2 hours. The mixture was adjusted to pH>12 with KOH, extracted with EtOAc. The extracts were washed with water and dried. The solvents were removed in vacuo to give 0209 (305 mg, 71.8%). The product was used in next step reaction without further purification.

### Step 7j: (R)—N,N-Dimethyl-3-(2-nitro-4-sulfamoylphenylamino)-4-(phenylthio)butanamide (Compound 0210)

A solution of 0209 (424 mg, 1.8 mmol), 4-Fluoro-3-nitro-benzenesulfonamide (396 mg, 1.8 mmol), and DIPEA (232 mg, 1.8 mmol) in DMF (10 mL) was stirred for 4 hours. The mixture was poured into water and extracted with EtOAc (50 ml). The extracts were washed with water, dried (Na₂SO₄), concentrated. The residue was subjected to flash column chromatography on silica gel eluting with 5% MeOH/CH₂Cl₂ to provide 0210 (680 mg, 87.2%). LC-MS: 439 [M+1]⁺. ¹H NMR (DMSO-d₆): δ 2.77 (s, 3H), 2.89 (s, 3H), 3.00 (m, 1H), 3.40 (d, J=6.5 Hz, 2H), 4.40 (b, 1H), 7.06 (d, J=10.0 Hz, 1H), 7.19 (m, 1H), 7.25 (m, 2H), 7.32 (m, 4H), 7.72 (m, 1H), 8.38 (d, J=2.3 Hz, 1H), 8.75 (d, J=10.0 Hz, 1H).

### Step 7k: (R)-4-(4-(Dimethylamino)-1-(phenylthio)butan-2-ylamino)-3-nitrobenzenesulfonamide (Compound 0211)

A mixture of compound 0210 (6.7 g, 15 mmol) and 1M BH3 in THF (30 ml) was stirred for 16 hours. To the resulting mixture were added MeOH (8 ml) and concentrated HCl (3 ml) and the mixture was stirred at 80° C. for 3 hours. The mixture was cooled to room temperature, adjusted to pH10 with 4M Na₂CO₃. To the mixture ethyl acetate (300 mL) was added. The separated organic layer was washed with water (70 ml), dried (MgSO4), filtered and concentrated. The residue was subjected to flash column chromatography on silica gel eluting with 20% MeOH/CH₂Cl₂ to provide 0211 (3.0 g, 46.3%). LC-MS: 425 [M+1]⁺. ¹H NMR (CDCl₃): δ 1.86 (m, 1H), 2.04 (m, 1H), 2.21 (s, 6H), 2.30 (m, 1H), 2.50 (m, 1H), 3.13 (d, J=5.7 Hz, 2H), 4.00 (m, 1H), 5.22 (br, 2H), 6.74 (d, J=9.3 Hz, 1H), 7.23 (m, 3H), 7.34 (m, 2H), 7.72 (d, J=9.3 Hz, 1H), 8.63 (s, 1H), 8.97 (d, J=8.1 Hz, 1H).

### Step 7l: (R)-Methyl 5-(2-(4-((4′-chlorobiphenyl-2-yl)methyl)piperazin-1-yl)-5-(4-(4-(dimethylamino)-1-(phenylthio)butan-2-ylamino)-3-nitrophenylsulfonylcarbamoyl)phenylamino)-5-oxopentanoate (Compound 0212-7)

A mixture of 0207-7 (549 mg, 1 mmol), 0211 (297 mg, 0.7 mmol), EDAC (390 mg, 2 mmol), and DMAP (244 mg, 2 mmol) in dichloromethane (20 ml) was stirred overnight at 25° C. The mixture was washed with saturated NH₄Cl (100 ml), dried (MgSO₄), filtered, and concentrated. The residue was subjected to flash column chromatography on silica gel eluting with 15% methanol/CH₂Cl₂ to afford 0212-7 (324 mg, 48.4%). LC-MS: 956 [M+1]⁺. ¹H NMR (DMSO-d₆+D₂O): δ 1.79 (m, 2H), 2.07 (m, 2H), 2.31 (m, 4H), 2.48 (m, 4H), 2.67 (s, 6H), 2.74 (m, 4H), 3.03 (m, 2H), 3.31 (m, 2H), 3.40 (s, 2H), 3.52 (s, 3H), 4.05 (m, 1H), 6.90 (m, 1H), 7.25 (m, 5H), 7.35 (m, 2H), 7.50 (m, 5H), 7.59 (m, 1H), 7.79 (m, 1H), 8.10 (d, J=9.0 Hz, 1H), 8.19 (s, 1H), 8.42 (d, J=1.8 Hz, 1H), 8.73 (br, 1H).

### Step 7m: (R)—N¹-(2-(4-((4′-Chlorobiphenyl-2-yl)methyl)piperazin-1-yl)-5-(4-(4-(dimethylamino)-1-(phenylthio)butan-2-ylamino)-3-nitrophenylsulfonylcarbamoyl)phenyl)-N⁵-hydroxyglutaramide (Compound 7)

Compound 0212-7 (100 mg, 0.1 mmol) was added into the saturated NH₂OH solution in methanol (0.56 mL, 1.76 mol/L). The mixture was reacted for 5 minutes with ultrasonication. Then the mixture was neutralized with diluted HOAc. The solvent was removed in vacuo. The residue was purified with preparative liquid chromatography to obtain 7 (20 mg, 20.9%) as a yellow solid. Mp: 146° C. ¹H NMR (DMSO-d₆+D₂O): δ 1.76 (m, 2H), 2.00 (br, 4H), 2.26 (m, 2H), 2.36 (m, 4H), 2.64 (m, 10H), 3.01 (m, 2H), 3.15 (m, 1H), 3.29 (m, 1H), 3.41 (m, 2H), 4.05 (m, 1H), 6.91 (m, 1H), 7.04 (m, 6H), 7.31 (m, 8H), 7.55 (m, 1H), 7.72 (m, 1H), 8.04 (s, 1H), 8.31 (s, 1H).

### Example 8

### Preparation of (R)—N′-(2-(4-((4′-chlorobiphenyl-2-yl)methyl)-piperazin-1-yl)-5-(4-(4-(dimethylamino)-1-(phenylthio)butan-2-ylamino)-3-nitrophenylsulfonylcarbamoyl)phenyl)-N⁶-hydroxyadipamide (Compound 8)

### Step 8a: tert-butyl 4-(4-((4′-chlorobiphenyl-2-yl)methyl)piperazin-1-yl)-3-(6-ethoxy-6-oxohexanamido)benzoate (Compound 0216-8)

The title compound 0216-8 was prepared (500 mg, 75.4%) from compound 0205 (500 mg, 1 mmol), DIEA (250 mg, 2 mmol), and ethyl 6-chloro-6-oxohexanoate (192 mg, 1 mmol) using a procedure similar to that described for compound 206-7 (Example 7): LC-MS: 634 [M+1]⁺. ¹H NMR (CDCl₃): δ 1.25 (t, J=7.4 Hz, 3H), 1.57 (s, 9H), 1.69 (m, 4H), 2.34 (m, 4H), 2.55 (br, 4H), 2.84 (br, 4H), 3.47 (s, 2H), 4.12 (q, J=7.4 Hz, 2H), 7.14 (q, J=2.1 Hz, 1H), 7.26 (m, 2H), 7.40 (m, 6H), 7.52 (m, 1H), 7.31 (dd, J=2.1, 8.1 Hz, 1H), 8.19 (br, 1H).

### Step 8b: 4-(4-((4′-Chlorobiphenyl-2-yl)methyl)piperazin-1-yl)-3-(6-ethoxy-6-oxohexanamido)benzoic acid (Compound 0207-8)

To the solution of 0206-8 (500 mg, 0.79 mmol) in CH₂Cl₂ (10 ml) was added trifluoroacetic acid (1 ml). The solution was stirred overnight at room temperature. The solvent was removed in vacuo to afford 0207-8 (380 mg, 83.2%). The product was used in next step reaction without further purification. LC-MS: 578 [M+1]⁺. ¹H NMR (CDCl₃): δ 1.22 (t, J=7.2 Hz, 3H), 1.62 (br, 4H), 2.30 (br, 4H), 2.93 (br, 4H), 3.19 (s, 2H), 3.54 (s, 2H), 4.03 (q, J=7.2 Hz, 2H), 4.47 (s, 2H), 6.98 (m, 1H), 7.24 (m, 3H), 7.30 (m, 1H), 7.45 (m, 4H), 7.57 (m, 1H), 7.74 (m, 1H), 8.20 (s, 1H), 8.58 (s, 1H).

### Step 8c: (R)-Ethyl 6-(2-(4-((4′-chlorobiphenyl-2-yl)methyl)piperazin-1-yl)-5-(4-(4-(dimethylamino)-1-(phenylthio)butan-2-ylamino)-3-nitrophenylsulfonylcarbamoyl)phenylamino)-6-oxohexanoate (Compound 0212-8)

A mixture of 0207-8 (480 mg, 0.8 mmol), 0211 (293 mg, 0.7 mmol), EDAC (191 mg, 1 mmol), and DMAP (122 mg, 1 mmol) in dichloromethane (20 mL) was stirred at 25° C. overnight. The mixture was washed with saturated NH₄Cl (100 mL), and dried (MgSO4), filtered, and concentrated. The residue was subjected to flash column chromatography on silica gel eluting with 15% methanol/CH₂Cl₂ to afford 0212-8 (420 mg, 60.0%). ¹H NMR (DMSO-d₆): δ 1.13 (t, J=7.4 Hz, 3H), 1.52 (br, 4H), 2.10 (m, 2H), 2.30 (m, 4H), 2.55 (m, 4H), 2.72 (s, 6H), 2.84 (m, 4H), 3.09 (m, 2H), 3.28 (m, 2H), 3.42 (m, 2H), 3.97 (q, J=7.4 Hz, 2H), 4.12 (s, 1H), 6.96 (m, 1H), 7.00 (m, 1H), 7.15 (m, 3H), 7.18 (m, 1H), 7.26 (m, 3H), 7.30 (m, 2H), 7.39 (m, 1H), 7.48 (m, 4H), 7.60 (m, 1H), 7.80 (m, 1H), 8.20 (m, 1H), 8.48 (m, 1H), 8.80 (s, 1H), 9.5 (br, 1H).

### Step 8d: (R)—N¹-(2-(4-((4′-Chlorobiphenyl-2-yl)methyl)piperazin-1-yl)-5-(4-(4-(dimethylamino)-1-(phenylthio)butan-2-ylamino)-3-nitrophenylsulfonylcarbamoyl)phenyl)-N⁶-hydroxyadipamide (Compound 8)

Compound 0212-8 (100 mg, 0.1 mmol) was added into the saturated NH₂OH solution in methanol (0.56 mL, 1.76 mol/L). The solution was sonicated for 5 minutes. Then the mixture was neutralized with acetic acid. Solvent was removed in vacuo. The residue was purified with preparative HPLC to afford compound 8 (20 mg, 20.6%) as a yellow solid. Mp.: 150° C. LC-MS: 971 [M+1]. ¹H NMR (DMSO-d₆+D₂O): δ 1.50 (br, 4H), 1.95 (m, 2H), 2.05 (m, 2H), 2.29 (m, 2H), 2.49 (br, 4H), 2.66 (s, 6H), 2.72 (br, 4H), 3.04 (m, 2H), 3.30 (m, 2H), 3.40 (m, 2H), 4.05 (m, 1H), 6.86 (d, J=9.6 Hz, 1H), 6.94 (d, J=8.1 Hz, 1H), 7.18 (m, 6H), 7.31 (m, 2H), 7.47 (m, 5H), 7.55 (d, J=8.1 Hz, 1H), 7.78 (d, J=9.0 Hz, 1H), 8.17 (br, 1H), 8.39 (s, 1H).

### Example 9

### Preparation of (R)—N¹-(2-(4-((4′-chlorobiphenyl-2-yl)methyl)piperazin-1-yl)-5-(4-(4-(dimethylamino)-1-(phenylthio)butan-2-ylamino)-3-nitrophenylsulfonylcarbamoyl)phenyl)-N⁸-hydroxyoctanediamide (Compound 9)

### Step 9a: tert-butyl 4-(4-((4′-chlorobiphenyl-2-yl)methyl)piperazin-1-yl)-3-(8-methoxy-8-oxooctanamido)benzoate (Compound 0206-9)

A mixture of compound 0205 (500 mg, 1 mmol) and DIEA (193 mg, 1.5 mmol) in 20 ml of CH₂Cl₂ was cooled to 0° C. To the solution methyl 8-chloro-8-oxooctanoate (216 mg, 1 mmol) was added. The mixture was warmed to room temperature and stirred for one hour. The solvent was removed in vacuo, and the residue was subjected to column chromatography on silica gel eluting with 25% EtOAc/petroleum ether to provide 0206-9 (630 mg, 92.7%). LC-MS: 648 [M+1]⁺. ¹H NMR (DMSO-d₆): δ 1.39 (m, 2H), 1.53 (s, 9H), 1.64 (m, 4H), 1.75 (m, 2H), 2.30 (m, 2H), 2.38 (m, 2H), 2.50 (b, 4H), 2.84 (t, J=5.7 Hz, 4H), 3.46 (s, 2H), 3.66 (s, 3H), 7.14 (m, 1H), 7.26 (m, 1H), 7.39 (m, 5H), 7.51 (m, 1H), 7.73 (m, 1H), 8.19 (s, 1H), 8.88 (m, 1H).

### Step 9b: 4-(4-((4′-Chlorobiphenyl-2-yl)methyl)piperazin-1-yl)-3-(8-methoxy-8-oxooctanamido)benzoic acid (Compound 0207-9)

To a solution of compound 0206-9 (720 mg, 1.1 mmol) in 10 ml of CH₂Cl₂ was added 1 ml of trifluoroacetic acid. The solution was stirred overnight at room temperature. The solvent was removed in vacuo to give product, 0207-9 (550 mg, 83.6%) which was used in next step reaction without further purification. LC-MS: 592 [M+1]⁺.

### Step 9c: (R)-Methyl 8-(2-(4-((4′-chlorobiphenyl-2-yl)methyl)piperazin-1-yl)-5-(4-(4-(dimethylamino)-1-(phenylthio)butan-2-ylamino)-3-nitrophenylsulfonylcarbamoyl)phenylamino)-8-oxooctanoate (Compound 0212-9)

A mixture of compound 0207-9 (540 mg, 0.9 mmol), 0211 (387 mg, 0.9 mmol), EDAC (382 mg, 2 mmol), and DMAP (244 mg, 2 mmol) in dichloromethane (20 mL) was stirred at 25° C. overnight. The mixture was washed with saturated NH₄Cl (100 ml), dried (MgSO4), filtered, and concentrated. The residue was subjected to flash column chromatography on silica gel eluting with 15% methanol/CH₂Cl₂ to afford 0212-9 (423 mg, 46.7%). LC-MS: 998 [M+1]⁺. ¹H NMR (DMSO-d₆): δ 1.26 (m, 4H), 1.50 (m, 4H), 2.07 (m, 2H), 2.22 (m, 4H), 2.46 (m, 4H), 2.67 (s, 6H), 2.76 (b, 4H), 3.04 (m, 2H), 3.40 (m, 2H), 3.54 (s, 3H), 4.05 (m, 1H), 6.89 (d, J=10.0 Hz, 1H), 6.98 (d, J=10.0 Hz, 1H), 7.18 (m, 1H), 7.25 (m, 3H), 7.29 (m, 2H), 7.37 (m, 2H), 7.47 (m, 5H), 7.58 (m, 1H), 7.81 (m, 1H), 8.12 (d, J=10.0 Hz, 1H), 8.22 (s, 1H), 8.44 (m, 1H), 8.67 (s, 1H).

### Step 9d: (R)—N¹-(2-(4-((4′-Chlorobiphenyl-2-yl)methyl)piperazin-1-yl)-5-(4-(4-(dimethylamino)-1-(phenylthio)butan-2-ylamino)-3-nitrophenylsulfonylcarbamoyl)phenyl)-N⁸-hydroxyoctanediamide (Compound 9)

Compound 0212-9 (300 mg, 0.3 mmol) was added into the saturated NH₂OH solution in methanol (1.7 ml, 1.76 mol/L). The mixture was sonicated for 5 minutes. Then the mixture was neutralized with acetic acid. The solvent was removed in vacuo. The residue was purified with preparative HPLC to afford compound 9 (17 mg, 5.7%). ¹H NMR (CD₃OD): δ 1.32 (m, 6H), 1.59 (m, 4H), 2.06 (m, 2H), 2.19 (m, 2H), 2.35 (m, 2H), 2.88 (s, 6H), 2.94 (b, 4H), 3.26 (m, 2H), 3.31 (m, 6H), 4.04 (s, 1H), 6.80 (m, 1H), 7.07 (m, 3H), 7.21 (m, 2H), 7.32 (m, 1H), 7.39 (m, 2H), 7.45 (m, 5H), 7.68 (m, 2H), 7.80 (m, 1H), 8.31 (s, 1H), 8.58 (m, 1H).

### Biological Assays:

As stated hereinbefore the derivatives defined in the present invention possess anti-proliferation activity. These properties may be assessed, for example, using one or more of the procedures set out below:

### (a) Bcl-2 and Bcl-xL Competition Binding (Fluorescence Polarization) Assay

### Background:

Bcl-2 and Bcl-xL proteins are antiapoptotic proteins whose biological function can be inhibited by proapototic proteins such as Bak, Bad and Bax through protein interaction. The interaction between antiapoptotic and proapototic proteins are mediated primarily by Bcl-2 homology (BH) 3 domain of Bak, Bad, Bax that bind to the hydrophobic groove of Bcl-2 and Bcl-xL. The demonstration of BH3 peptide alone induce apoptosis encourage the possibility of design or identify a chemical compound that mimics the function of BH3 peptide by blocking Bcl-2 or Bcl-xLs' interaction with their downstream binding partners. These chemical compounds are expected to bind to the hydrophobic groove of Bcl-xL or Bcl-2 proteins with high affinity. A labeled BH3 peptide can be used for competition binding and to monitor the interaction between compounds and Bcl-2 and Bcl-xL proteins.

### Rational and Method:

A 26-mer fluorescein labeled BH3 peptide (NLWAAQRYGRELRRMSDKFVD) was purchase from CalBiochem (197216). The interaction between Bcl-xL or Bcl-2 and peptide forms the basis for the fluorescence polarization assay. A free and fast-tumbling fluoresein labeled BH3 peptide emits random light with respect to the plane of polarization plane of excited light, resulting in a lower polarization degree (mP) value. When the peptide is bound to Bcl-xl or Bcl-2, the complex tumble slower and the emitted light is polarized, resulting in a higher mP value. This binding assay was performed in 96-well plate and with each assay contained 1 and 100 nM of labeled peptide and purified Bcl-xL (R&D System, 894-BX-050) or Bcl-2 protein (R&D System, 827-BC-050) respectively. The assay buffer contained 120 mM sodium phosphate (pH 7.55), 0.01% BSA and 0.1% sodium azide. Compounds were diluted in DMSO and added to the final assay with concentration range from 20 uM to 2 nM. mP value was determined by BioTek Synergy II with background subtraction after 3 hours of incubation at room temperature.

(b) An In Vitro Assay which Determines the Ability of a Test Compound to Inhibit HDAC Enzymatic Activity.

HDAC inhibitors were screened using an HDAC fluorimetric assay kit (AK-500, Biomol, Plymouth Meeting, Pa.). Test compounds were dissolved in dimethylsulphoxide (DMSO) to give a 20 mM working stock concentration. Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm. Each assay was setup as follows: Defrosted all kit components and kept on ice until use. Diluted HeLa nuclear extract 1:29 in Assay Buffer (50 mM Tris/Cl, pH 8.0, 137 mM NaCl, 2.7 mM KCl, 1 mM MgCl2). Prepared dilutions of Trichostatin A (TSA, positive control) and tested compounds in assay buffer (5× of final concentration). Diluted Fluor de Lys™ Substrate in assay buffer to 100 uM (50 fold=2× final). Diluted Fluor de Lys™ developer concentrate 20-fold (e.g. 50 μl plus 950 μl Assay Buffer) in cold assay buffer. Second, diluted the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Added Assay buffer, diluted trichostatin A or test inhibitor to appropriate wells of the microtiter plate. Added diluted HeLa extract or other HDAC sample to all wells except for negative controls. Allowed diluted Fluor de Lys™ Substrate and the samples in the microtiter plate to equilibrate to assay temperature (e.g. 25 or 37° C. Initiated HDAC reactions by adding diluted substrate (25 μl) to each well and mixing thoroughly. Allowed HDAC reactions to proceed for 1 hour and then stopped them by addition of Fluor de Lys™ Developer (50 μl). Incubated plate at room temperature (25° C.) for 10-15 min. Read samples in a microtiter-plate reading fluorimeter capable of excitation at a wavelength in the range 350-380 nm and detection of emitted light in the range 440-460 nm.

The following TABLE 10-B lists compounds representative of the invention and their activity in HDAC and Bcl-2 assays. In these assays, the following grading was used: I≧10 μM, 10 μM>II>1 μM, 1 μM>III>0.1 μM, and IV≦0.1 μM for IC₅₀.

TABLE 10-B

| Compound No. | HDAC | Bcl-2 |
|---|---|---|
| 1 | II | IV |
| 2 | III | |
| 3 | III | |
| 4 | III | |
| 5 | III | |

TABLE 11-A
SECTION 11:

| | |
|---|---|
| | (XIV) |
| 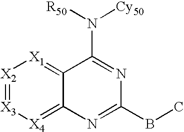 | |

| Compound # | Structure |
|---|---|
| 1 | 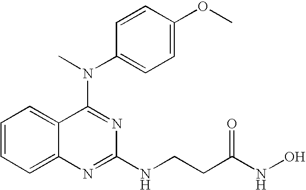 |
| 2 | 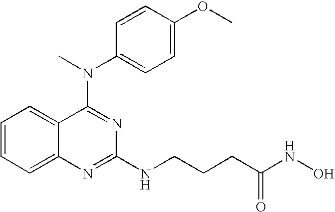 |
| 3 | 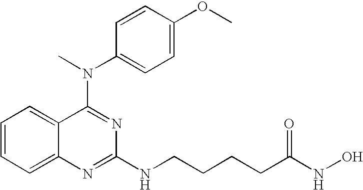 |
| 4 | 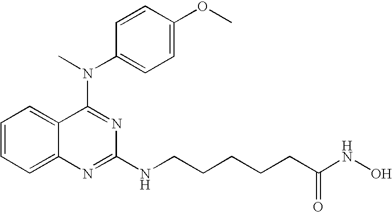 |
| 5 | 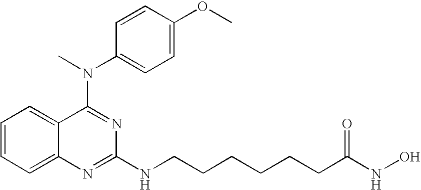 |
| 6 | 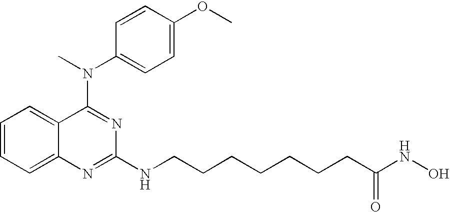 |
| 7 | 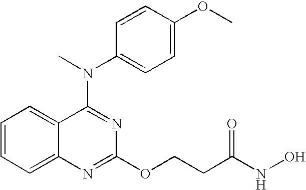 |
| 8 | 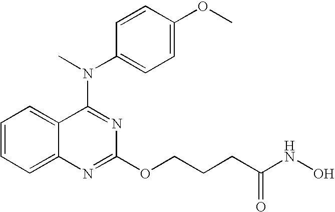 |
| 9 | 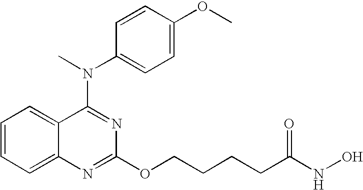 |
| 10 | 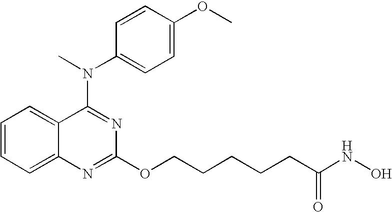 |
| 11 | 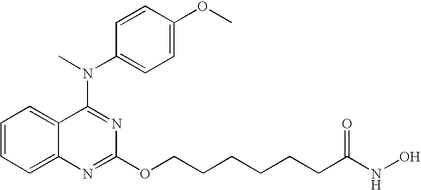 |
| 12 | 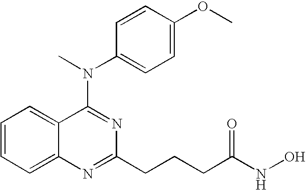 |
| 13 | 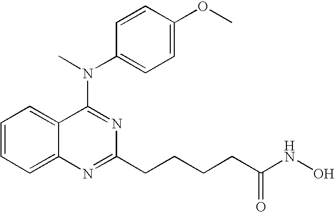 |
| 14 | 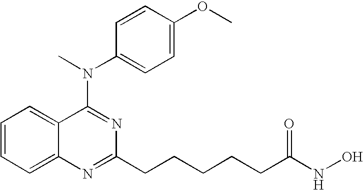 |
| 15 | 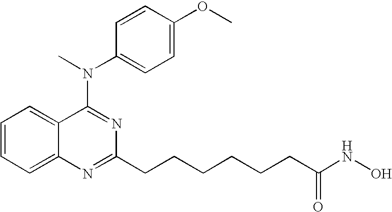 |
| 16 | 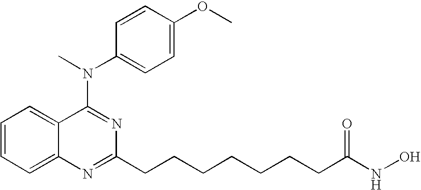 |
| 17 | 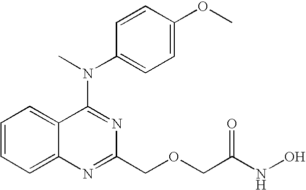 |
| 18 | 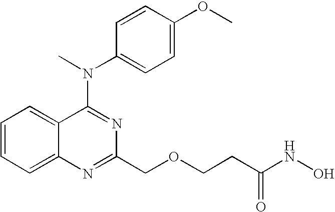 |
| 19 | 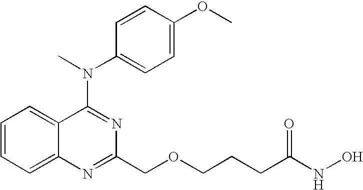 |
| 20 | 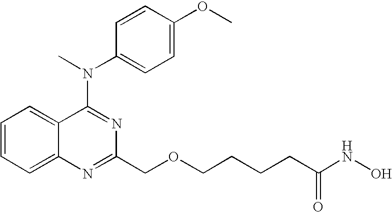 |
| 21 | 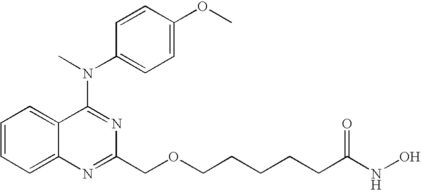 |
| 22 | 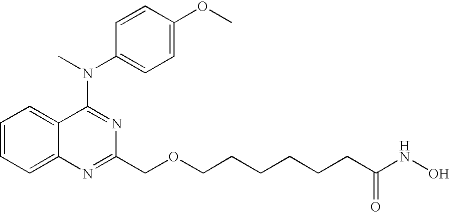 |
| 23 | 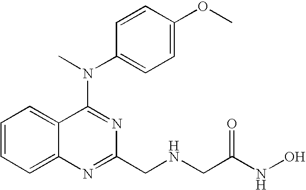 |
| 24 | 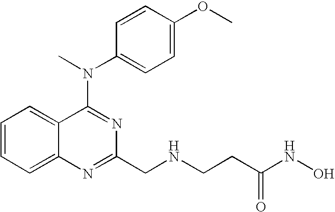 |
| 25 | 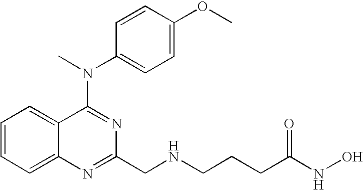 |
| 26 | 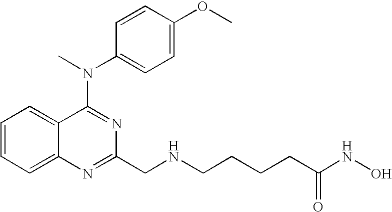 |
| 27 | 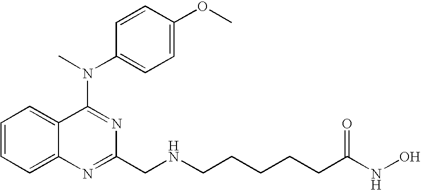 |
| 28 | 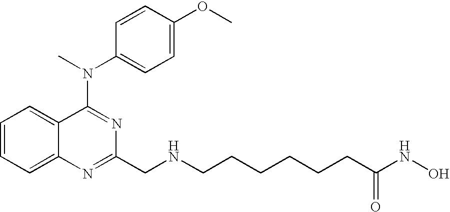 |
| 29 | 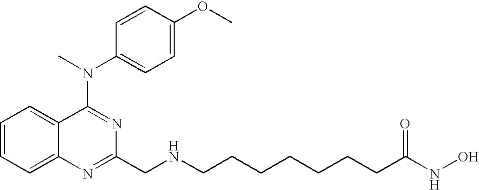 |

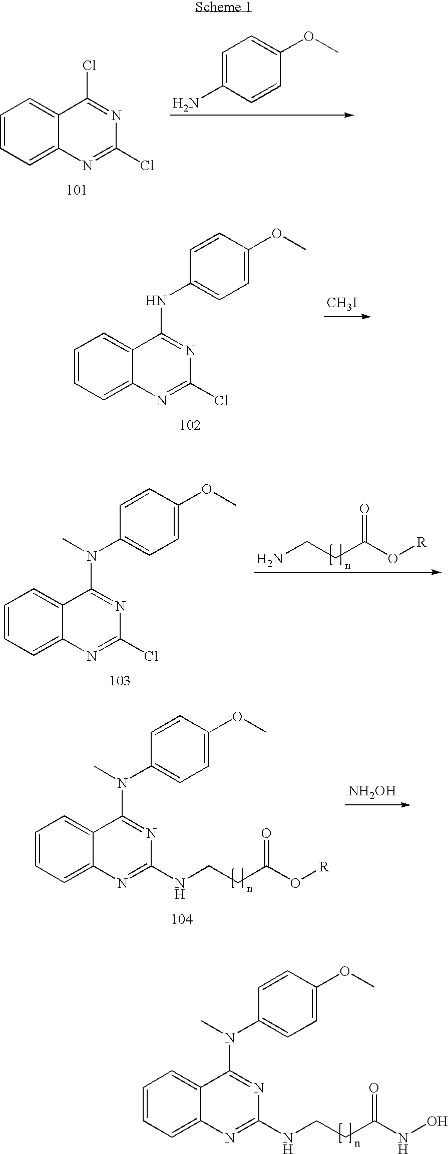

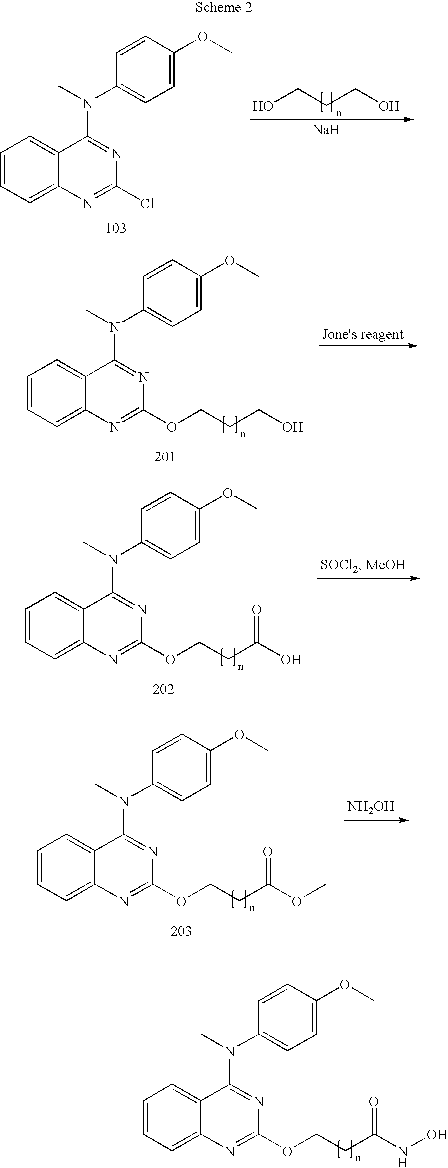

### Example 1

### Preparation of N-hydroxy-6-(4-((4-methoxyphenyl)(methyl)amino)quinazolin-2-ylamino)hexanamide (Compound 4)

### Step 1a. 2-Chloro-N-(4-methoxyphenyl)quinazolin-4-amine (Compound 102)

A mixture of compound 2,4-dichloroquinazoline 101 (9.9 g, 50 mmol) and compound 4-methoxybenzenamine (6.15 g, 50 mmol) in methanol was stirred at room temperature for 2 h. The reaction was evaporated and the residue was purified by column chromatography using ethyl acetate/petroleum ether (5/1) as eluent to give compound 102 (8.1 g, 55%): LC-MS: 286 [M+1]⁺.

### Step 1b. 2-Chloro-N-(4-methoxyphenyl)-N-methylquinazolin-4-amine (Compound 103)

To a solution of compound 102 (8.1 g, 28.4 mmol) in DMF (150 mL) was added NaH (1.25 g, 31.3 mmol). The reaction mixture was stirred for a few minutes and CH₃I (6.05 g, 42.6 mmol) was then added. After the addition, the mixture was stirred at room temperature for 18 h. The reaction was diluted with ethyl acetate and washed with water and brine, dried and concentrated to yield the crude product which was purified by column chromatography using ethyl acetate/petroleum ether (5/1) as eluent to give compound 103 as a yellow solid (7.2 g, 51% yield): LC-MS: 300 [M+1].

### Step 1c. Methyl 6-(4-((4-methoxyphenyl)(methyl)amino)quinazolin-2-ylamino)hexanoate (Compound 104-4)

KOH (248.6 mg, 4.44 mmol) was added to a solution of methyl 6-aminohexanoate hydrogen chloride (0.806 g, 4.44 mmol) in methanol (10 mL) and the mixture was stirred at room temperature for 10 min. Solvent was then removed and DMA (10 mL) and compound 103 (0.19 g, 0.635 mmol) were added. The mixture was stirred at 120° C. for 3 h. DMA was evaporated under reduce pressure and 50 mL ethyl acetate was added. The mixture was washed with water, dry with anhydrous Na₂SO₄, and concentrated to obtain compound 104-4 as a white solid (170 mg, 65%): LC-MS: 409 [M+1]⁻.

### Step 1d. N-Hydroxy-6-(4-((4-methoxyphenyl)(methyl)amino)quinazolin-2-ylamino)hexanamide (Compound 4)

Preparation of the solution of hydroxylamine in methanol: hydroxylamine hydrochloride (4.67 g, 67 mmol) was dissolved in methanol (24 mL) to form solution A. Potassium hydroxide (5.61 g, 100 mmol) was dissolved in methanol (14 mL) to form solution B. Solution A was cooled to 0° C., and solution B was added into solution A dropwise. The mixture was stirred for 30 minutes at 0° C. The precipitate was filtered off and the filtrate formed the solution of hydroxylamine in methanol.

To a flask containing compound 104-4 (340 mg, 0.831 mmol) was added the solution of hydroxylamine in methanol (5.0 mL). The mixture was stirred at room temperature for 1 hour and was then adjusted to PH 7 with the addition of acetic acid. The mixture was concentrated to give a residue which was filtered and washed with water to afford the product 4 as a white solid (150 mg, 44%): LC-MS: 410 [M+1]; ¹H NMR (DMSO-d₆): δ 10.35 (s, 1H), 7.31 (m, 2H), 7.15 (d, J=9.0 Hz, 2H), 6.96 (d, J=9.0 Hz, 2H), 6.76 (m, 2H), 6.61 (m, 1H), 3.77 (s, 3H), 3.42 (s, 3H), 1.97 (t, J=7.2 Hz, 2H), 1.58 (m, 4H), 1.35 (m, 2H); ¹H NMR (DMSO-d⁶+D₂O): δ 7.30 (m, 2H), 7.22 (d, J=9.0 Hz, 2H), 6.93 (d, J=9.0 Hz, 2H), 6.80 (m, 1H), 6.77 (m, 1H), 3.73 (s, 3H), 3.38 (s, 3H), 3.31 (t, J=7.2 Hz, 2H), 1.94 (t, J=7.2 Hz, 2H), 1.52 (m, 4H), 1.32 (m, 2H).

### Example 2

### Preparation of N-hydroxy-7-(4-((4-methoxyphenyl)(methyl)amino)quinazolin-2-ylamino)heptanamide (Compound 5)

### Step 2a. Methyl 7-(4-((4-methoxyphenyl)(methyl)amino)quinazolin-2-ylamino)heptanoate (Compound 104-5)

The title compound 104-5 was prepared (400 mg, 89%) from compound 103 (306 mg, 1.022 mmol), ethyl 7-aminoheptanoate hydrogen chloride (1.5 g, 7.156 mmol) and KOH (400 mg, 7.156 mmol) using a procedure similar to that described for compound 104-4 (Example 1): LC-MS: 437 [M+1]⁺.

### Step 2b. N-Hydroxy-7-(4-((4-methoxyphenyl)(methyl)amino)quinazolin-2-ylamino)heptanamide (Compound 5)

The title compound 5 was prepared (90 mg, 23%) from compound 104-5 (400 mg, 0.197 mmol) and freshly prepared hydroxylamine methanol solution (5 mL) using a procedure similar to that described for compound 4 (Example 1): LC-MS: 424 [M+1]⁺. ¹H NMR (DMSO-d₆): δ 10.37 (s, 1H), 8.72 (s 1H), 7.32 (m, 2H), 7.15 (d, J=8.7 Hz, 2H), 6.97 (d, J=8.7 Hz, 2H), 6.81 (m, 2H), 6.65 (m, 1H), 3.78 (s, 3H), 3.43 (s, 4H), 1.97 (t, J=7.2 Hz, 2H), 1.55 (m, 4H), 1.33 (m, 4H); ¹H NMR (DMSO-d₆+D₂O): δ 7.31 (m, 2H), 7.14 (d, J=8.4 Hz, 2H), 6.96 (d, J=8.4 Hz, 2H), 6.81 (m, 1H), 6.63 (m, 1H), 3.77 (s, 3H), 3.41 (s, 3H), 3.34 (m, 2H), 1.96 (t, J=7.2 Hz 2H), 1.56 (m, 4H), 1.33 (m, 4H).

### Example 3

### Preparation of N-hydroxy-8-(4-((4-methoxyphenyl)(methyl)amino)quinazolin-2-ylamino)octanamide (Compound 6)

### Step 3a. Methyl 8-(4-((4-methoxyphenyl)(methyl)amino)quinazolin-2-ylamino)octanoate (Compound 104-6)

The title compound 104-6 was prepared (114 mg, 26%) from compound 103 (0.299 g, 1 mmol), methyl 8-aminooctanoate hydrogen chloride (6.51 g, 31.05 mmol) and KOH (1.739 g, 31.05 mmol) using a procedure similar to that described for compound 104-4 (Example 1): LC-MS: 437 [M+1]⁺.

### Step 3b. N-Hydroxy-8-(4-((4-methoxyphenyl)(methyl)amino)quinazolin-2-ylamino)octanamide (Compound 6)

The title compound 6 was prepared (21 mg, 18%) from compound 104-6 (114 mg, 0.261 mmol) and freshly prepared hydroxylamine methanol solution (2 mL) using a procedure similar to that described for compound 4 (Example 1): LC-MS: 438 [M+1]⁺. ¹H NMR (DMSO-d₆): δ 10.32 (s, 1H), 8.66 (s, 1H), 7.05 (m, 2H), 7.22 (m, 2H), 7.00 (m, 2H), 6.76 (m, 2H), 3.78 (s, 3H), 3.47 (s, 3H), 3.38 (m, 2H), 1.94 (t, J=7.5 Hz, 2H), 1.62 (m, 2H), 1.49 (m, 2H), 1.32 (m, 6H).

### Example 4

### Preparation of N-hydroxy-5-(4-((4-methoxyphenyl)(methyl)amino)quinazolin-2-yloxy)pentanamide (Compound 9)

### Step 4a. 5-(4-((4-Methoxyphenyl)(methyl)amino)quinazolin-2-yloxy)pentan-1-ol (Compound 201-9)

NaH (0.6 g, 0.015 mol) was added into the pentane-1,5-diol (10.4 g, 0.1 mol) at 70° C. with stir. Compound 103 was added and the mixture was stirred at 70° C. for 3 h. After reaction, the mixture was diluted with ethyl acetate and washed with water and brine, dried and concentrated to afford compound 201-9 (1.768 g, 48%): LC-MS: 368 [M+1]⁻.

### Step 4b. 5-(4-((4-Methoxyphenyl)(methyl)amino)quinazolin-2-yloxy)pentanoic acid (Compound 202-9)

To a solution of compound 201-9 (1.768 g, 48 mmol) in acetone (150 mL) at 0° C. was added Jone's reagent (10 mL) dropwise. After addition, the mixture was stirred at room temperature for 1 h. Isopropyl alcohol (10 mL) was added and stirred. The resulting solid was removed by filtration and the filtrate was evaporated to leave a residue which was extracted with ethyl acetate. The ethyl acetate extract was washed with water and brine, dried and concentrated to afford compound 202-9 (1.44 g, 79%). LC-MS: 382 [M+1].

### Step 4c. Methyl 5-(4-((4-methoxyphenyl)(methyl)amino)quinazolin-2-yloxy)pentanoate (Compound 203-9)

To a solution of compound 202-9 (1.437 g, 3.8 mmol) in MeOH (25 mL) at 0° C. was SOCl₂ (2 mL) dropwise. After addition, the mixture was stirred at room temperature for 16 h. The reaction was evaporated to give compound 203-9 (1.4 g, 94%): LC-MS: 396 [M+1]⁻.

### Step 4d. N-Hydroxy-5-(4-((4-methoxyphenyl)(methyl)amino)quinazolin-2-yloxy)pentanamide (Compound 9)

The title compound 9 was prepared (98 mg, 50%) from compound 203-9 (197.5 mg, 0.5 mmol) and freshly prepared hydroxylamine methanol solution (5 mL) using a procedure similar to that described for compound 4 (Example 1): LC-MS: 397.1 [M+1]⁻; ¹H NMR (DMSO-d₆): δ 10.39 (s, 1H), 8.71 (s, 1H), 7.51 (m, 2H), 7.23 (d, J=9.0 Hz, 2H), 7.00 (d, J=9.0 Hz, 2H), 6.89 (m, 2H), 4.35 (t, J=6.0 Hz, 2H), 3.79 (s, 3H), 3.46 (s, 3H), 2.05 (t, J=6.9 Hz, 2H), 1.72 (m, 4H).

### Example 5

### Preparation of N-hydroxy-6-(4-((4-methoxyphenyl)(methyl)amino)quinazolin-2-yloxy)hexanamide (Compound 10)

### Step 5a. 5-(4-((4-Methoxyphenyl)(methyl)amino)quinazolin-2-yloxy)pentan-1-ol (Compound 201-10)

The title compound 201-10 was prepared (2.204 g, 51%) from compound 103 (2.99 g, 0.01 mol), NaH (0.6 g, 0.015 mol) and hexane-1,6-diol (11.8 g, 0.1 mol) using a procedure similar to that described for compound 201-9 (Example 4): LC-MS: 382 [M+1]⁻.

### Step 5b. 6-(4-((4-Methoxyphenyl)(methyl)amino)quinazolin-2-yloxy)hexanoic acid (Compound 202-10)

The title compound 202-10 was prepared (2.204 g, 96%) from compound 201-10 ((2.204 g, 5.8 mmol) and Jone's reagent (10 mL) using a procedure similar to that described for compound 202-9 (Example 4): LC-MS: 396 [M+1]⁺.

### Step 5c. Methyl 6-(4-((4-methoxyphenyl)(methyl)amino)quinazolin-2-yloxy)hexanoate (Compound 203-10)

The title compound 203-10 was prepared (1.995 g, 88%) from compound 202-10 (2.2 g, 5.54 mmol), SOCl₂ (3 mL) and MeOH (35 mL) using a procedure similar to that described for compound 203-9 (Example 4): LC-MS: 410 [M+1]⁺.

### Step 5d. N-Hydroxy-6-(4-((4-methoxyphenyl)(methyl)amino)quinazolin-2-yloxy)hexanamide (Compound 10)

The title compound 10 was prepared (35 mg, 17%) from compound 203-10 (204.5 mg, 0.5 mmol) and freshly prepared hydroxylamine methanol solution (5 mL) using a procedure similar to that described for compound 4 (Example 1): LC-MS: 411 [M+1]⁺; ¹H NMR (DMSO-d₆): δ 10.37 (s, 1H), 8.69 (s, 1H), 7.52 (m, 2H), 7.23 (d, J=9.0 Hz, 2H), 7.00 (d, J=9.0 Hz, 2H), 6.89 (m, 2H), 4.34 (t, J=6.3 Hz, 2H), 3.79 (s, 3H), 3.46 (s, 3H), 2.00 (m, 2H), 1.76 (m, 2H), 1.59 (m, 2H), 1.43 (m, 2H).

### Example 6

### Preparation of N-hydroxy-7-(4-((4-methoxyphenyl)(methyl)amino)quinazolin-2-yloxy)heptanamide (Compound 11)

### Step 6a. 7-(4-((4-Methoxyphenyl)(methyl)amino)quinazolin-2-yloxy)heptan-1-ol (Compound 201-11)

The title compound 201-11 was prepared (652 mg, 17%) from compound 103 (2.873 g, 9.6 mmol), NaH (0.585 g, 14.6 mmol) and heptane-1,7-diol (7.622 g, 57.7 mmol) using a procedure similar to that described for compound 201-9 (Example 4): LC-MS: 396 [M+1]⁺.

### Step 6b. 7-(4-((4-Methoxyphenyl)(methyl)amino)quinazolin-2-yloxy)heptanoic acid (Compound 202-11)

The title compound 202-11 was prepared (657 mg, 97%) from compound 201-11 (652 mg, 1.65 mmol) and Jone's reagent (5 mL) using a procedure similar to that described for compound 202-9 (Example 4): LC-MS: 410 [M+1]⁺.

### Step 6c. Methyl 7-(4-((4-methoxyphenyl)(methyl)amino)quinazolin-2-yloxy)heptanoate (Compound 203-11)

The title compound 203-11 was prepared (600 mg, 88%) from compound 202-11 (657 mg, 1.6 mmol) and SOCl₂ (1 mL) using a procedure similar to that described for compound 203-9 (Example 4): LC-MS: 424 [M+1]⁺.

### Step 6d. N-Hydroxy-7-(4-((4-methoxyphenyl)(methyl)amino)quinazolin-2-yloxy)heptanamide (Compound 11)

The title compound 11 was prepared (200 mg, 33%) from compound 203-11 (600 mg, 1.42 mmol) and freshly prepared solution of hydroxylamine in methanol (10.0 mL) using a procedure similar to that described for compound 9 (Example 4): LC-MS: 438 [M+1]⁻; ¹H NMR (DMSO-d₆): δ 10.34 (s, 1H), 8.66 (s, 1H), 7.52 (m, 2H), 7.23 (d, J=9.0 Hz, 2H), 7.00 (d, J=9.0 Hz, 2H), 6.89 (m, 2H), 4.34 (t, J=6.0 Hz, 2H), 3.79 (s, 3H), 3.46 (s, 3H), 1.97 (t, J=7.2 Hz, 2H), 1.76 (m, 2H), 1.54 (m, 2H), 1.43 (m, 2H), 1.35 (m, 2H).

### Biological Assays:

As stated hereinbefore the derivatives defined in the present invention possess anti-proliferation activity. These properties may be assessed, for example, using one or more of the procedures set out below:

(a) An In Vitro Assay which Determines the Ability of a Test Compound to Inhibit a Receptor Tyrosine Kinase.

The ability of compounds to inhibit receptor kinase (VEGFR2 and PDGFR-beta) activity was assayed using HTScan™ Receptor Kinase Assay Kits (Cell Signaling Technologies, Danvers, Mass.). VEGFR2 tyrosine kinase was produced using a baculovirus expression system from a construct containing a human VEGFR2 cDNA kinase domain (Asp805-Val356) (GenBank accession No. AF035121) fragment amino-terminally fused to a GST-HIS6-Thrombin cleavage site. PDGFR-beta tyrosine kinase was produced using a baculovirus expression system from a construct containing a human PDGFR-beta c-DNA (GenBank Accession No. NM_{—}002609) fragment (Arg561-Leu1106) amino-terminally fused to a GST-HIS6-Thrombin cleavage site. The proteins were purified by one-step affinity chromatography using glutathione-agarose. An anti-phosphotyrosine monoclonal antibody, P-Tyr-100, was used to detect phosphorylation of biotinylated substrate peptides (VEGFR2, Biotin-Gastrin Precursor (Tyr87); PDGFR-β, Biotinylated-FLT3 (Tyr589)). Enzymatic activity was tested in 60 mM HEPES, 5 mM MgCl2 5 mM MnCl2 200 μM ATP, 1.25 mM DTT, 3 μM Na3VO4, 1.5 mM peptide, and 50 ng EGF Receptor Kinase. Bound antibody was detected using the DELFIA system (PerkinElmer, Wellesley, Mass.) consisting of DELFIA® Europium-labeled Anti-mouse IgG (PerkinElmer, #AD0124), DELFIA® Enhancement Solution (PerkinElmer, #1244-105), and a DELFIA® Streptavidin coated, 96-well Plate (PerkinElmer, AAAND-0005). Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm.

Test compounds were dissolved in dimethylsulphoxide (DMSO) to give a 20 mM working stock concentration. Each assay was setup as follows: Added 100 μl of 10 mM ATP to 1.25 ml 6 mM substrate peptide. Diluted the mixture with dH₂0 to 2.5 ml to make 2×ATP/substrate cocktail ([ATP]=400 mM, [substrate]=3 mM). Immediately transfer enzyme from −80° C. to ice. Allowed enzyme to thaw on ice. Microcentrifuged briefly at 4° C. to bring liquid to the bottom of the vial. Returned immediately to ice. Added 10 μl of DTT (1.25 mM) to 2.5 ml of 4×HTScan™ Tyrosine Kinase Buffer (240 mM HEPES pH 7.5, 20 mM MgCl₂, 20 mM MnCl, 12 mM NaVO₃) to make DTT/Kinase buffer. Transfer 1.25 ml of DTT/Kinase buffer to enzyme tube to make 4× reaction cocktail ([enzyme]=4 ng/μL in 4× reaction cocktail). Incubated 12.5 μl of the 4× reaction cocktail with 12.5 μl/well of prediluted compound of interest (usually around 10 μM) for 5 minutes at room temperature. Added 25 μl of 2×ATP/substrate cocktail to 25 μl/well preincubated reaction cocktail/compound. Incubated reaction plate at room temperature for 30 minutes. Added 50 μl/well Stop Buffer (50 mM EDTA, pH 8) to stop the reaction. Transferred 25 μl of each reaction and 75 μl dH₂O/well to a 96-well streptavidin-coated plate and incubated at room temperature for 60 minutes. Washed three times with 200 μl/well PBS/T (PBS, 0.05% Tween-20). Diluted primary antibody, Phospho-Tyrosine mAb (P-Tyr-100), 1:1000 in PBS/T with 1% bovine serum albumin (BSA). Added 100 μl/well primary antibody. Incubated at room temperature for 60 minutes. Washed three times with 200 μl/well PBS/T. Diluted Europium labeled anti-mouse IgG 1:500 in PBS/T with 1% BSA. Added 100 μl/well diluted antibody. Incubated at room temperature for 30 minutes. Washed five times with 200 μl/well PBS/T. Added 100 μl/well DELFIA® Enhancement Solution. Incubated at room temperature for 5 minutes. Detected 615 nm fluorescence emission with appropriate Time-Resolved Plate Reader.

(b) An In Vitro Assay which Determines the Ability of a Test Compound to Inhibit HDAC Enzymatic Activity.

HDAC inhibitors were screened using an HDAC fluorimetric assay kit (AK-500, Biomol, Plymouth Meeting, Pa.). Test compounds were dissolved in dimethylsulphoxide (DMSO) to give a 20 mM working stock concentration. Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm. Each assay was setup as follows: Defrosted all kit components and kept on ice until use. Diluted HeLa nuclear extract 1:29 in Assay Buffer (50 mM Tris/Cl, pH 8.0, 137 mM NaCl, 2.7 mM KCl, 1 mM MgCl2). Prepared dilutions of Trichostatin A (TSA, positive control) and tested compounds in assay buffer (5× of final concentration). Diluted Fluor de Lys™ Substrate in assay buffer to 100 uM (50 fold=2× final). Diluted Fluor de Lys™ developer concentrate 20-fold (e.g. 50 μl plus 950 μl Assay Buffer) in cold assay buffer. Second, diluted the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Added Assay buffer, diluted trichostatin A or test inhibitor to appropriate wells of the microtiter plate. Added diluted HeLa extract or other HDAC sample to all wells except for negative controls. Allowed diluted Fluor de Lys™ Substrate and the samples in the microtiter plate to equilibrate to assay temperature (e.g. 25 or 37° C. Initiated HDAC reactions by adding diluted substrate (25 μl) to each well and mixing thoroughly. Allowed HDAC reactions to proceed for 1 hour and then stopped them by addition of Fluor de Lys™ Developer (50 μl). Incubated plate at room temperature (25° C.) for 10-15 min. Read samples in a microtiter-plate reading fluorimeter capable of excitation at a wavelength in the range 350-380 nm and detection of emitted light in the range 440-460 nm.

### (c) Activity Against Tumor Vasculature Measured by Fluorescent Dye.

The following experiment demonstrates the ability of the compounds to damage tumor vasculature.

Tumor functional vascular volume in CaNT tumor-bearing mice is measured using the fluorescent dye Hoechst 33342 according to the method of Smith et al (Brit J Cancer 57, 247-253, 1988). The fluorescent dye is dissolved in saline at 6.25 mg/ml and injected intravenously at 10 mg/kg 6 hours or 24 hours after intraperitoneal drug treatment. One minute later, animals are killed and tumours excised and frozen; 10 μm sections are cut at 3 different levels and observed under UV illumination using an Olympus microscope equipped with epifluorescence. Blood vessels are identified by their-fluorescent outlines and vascular volume is quantified using a point scoring system based on that described by Chalkley, (J Natl Cancer Inst, 4, 47-53, 1943). All estimates are based on counting a minimum of 100 fields from sections cut at the 3 different levels. Results are expressed as percentage reduction in vascular volume compared to control.

TABLE 12-A
SECTION 12:

| | |
|---|---|
| | (XV) |
| 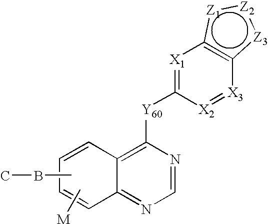 | |

| Compound # | Structure |
|---|---|
| 1 | 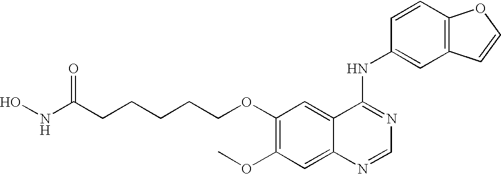 |
| 2 | 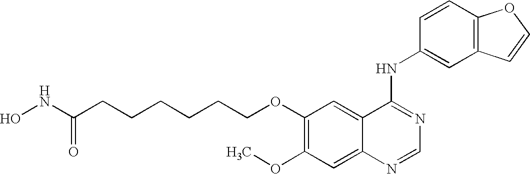 |
| 3 | 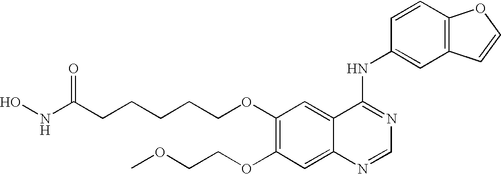 |
| 4 | 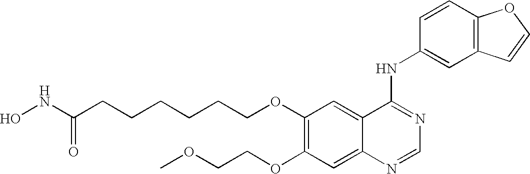 |
| 5 | 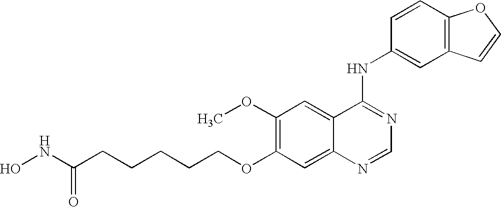 |
| 6 | 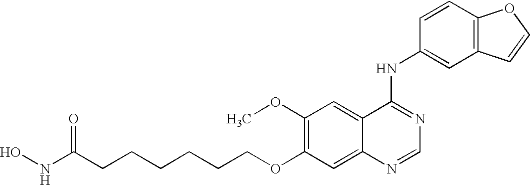 |
| 7 | 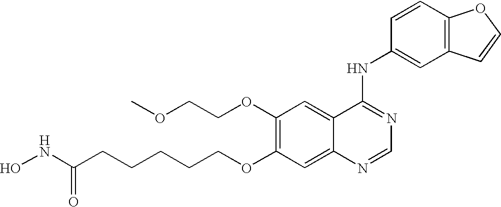 |
| 8 | 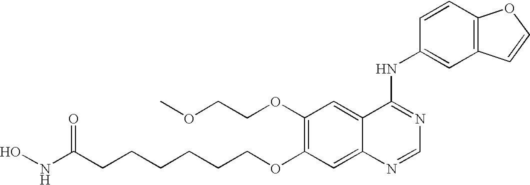 |
| 9 | 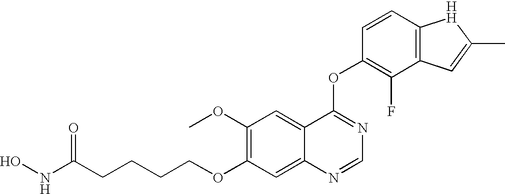 |
| 10 | 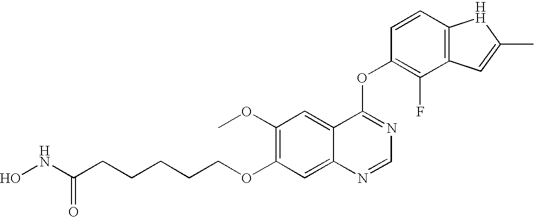 |
| 11 | 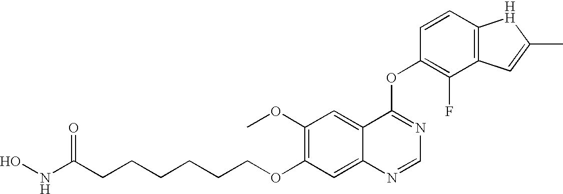 |
| 12 | 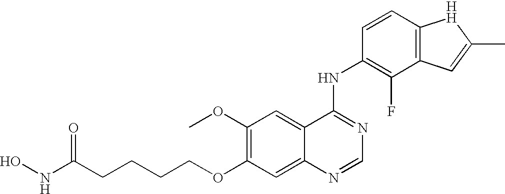 |
| 13 | 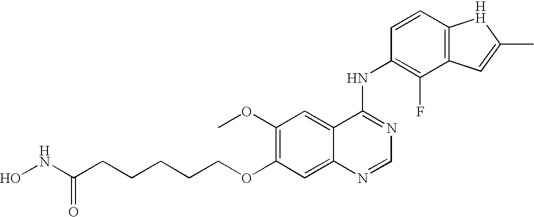 |
| 14 | 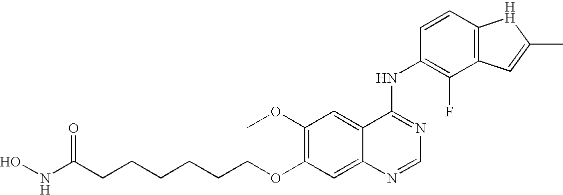 |
| 15 | 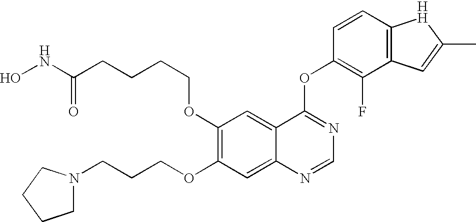 |
| 16 | 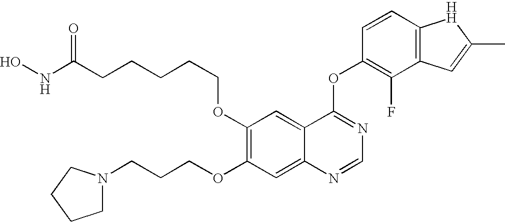 |
| 17 | 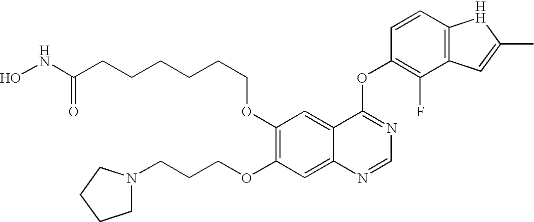 |
| 18 | 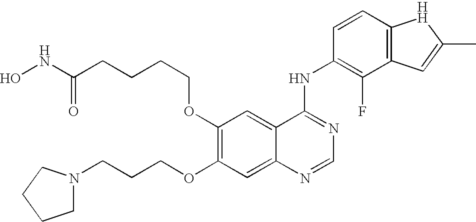 |
| 19 | 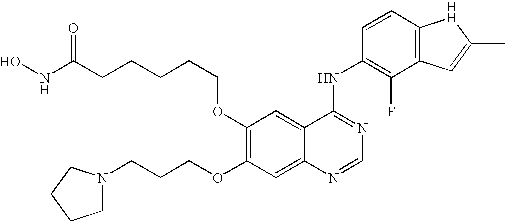 |
| 20 | 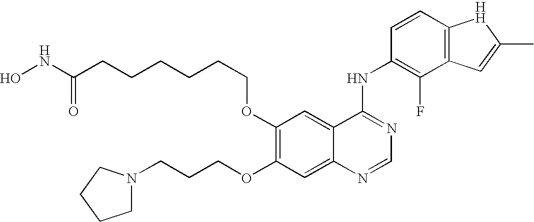 |

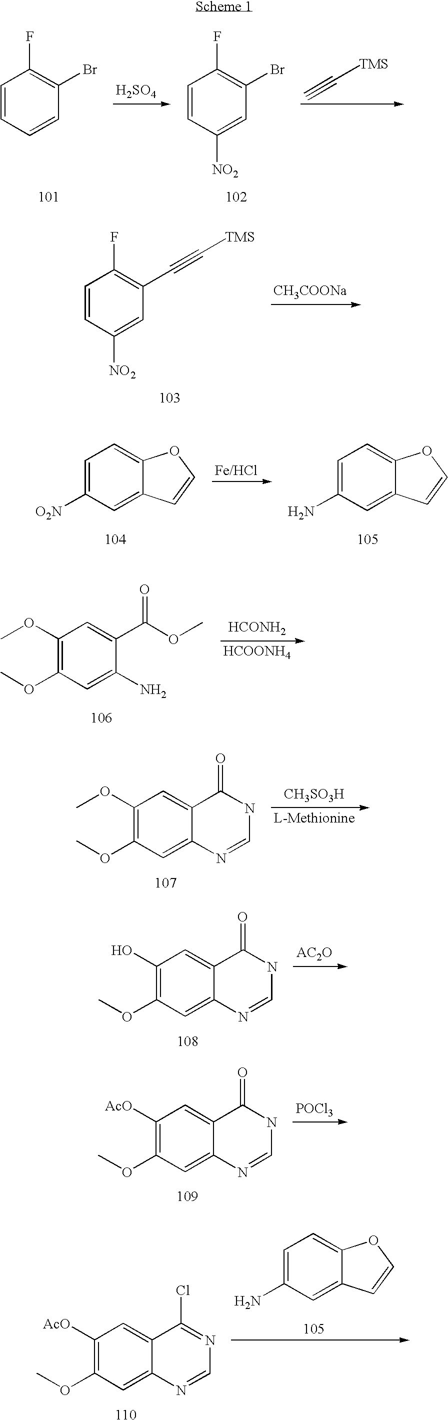

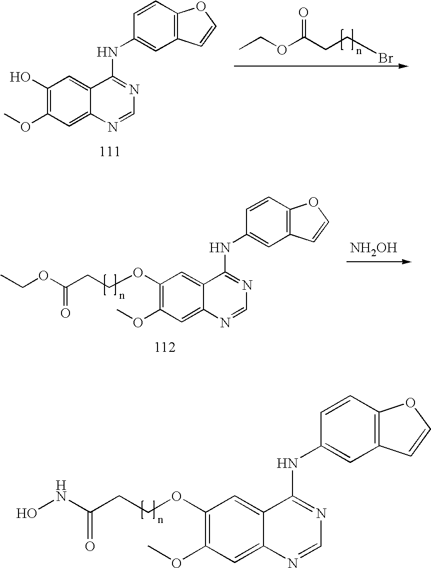

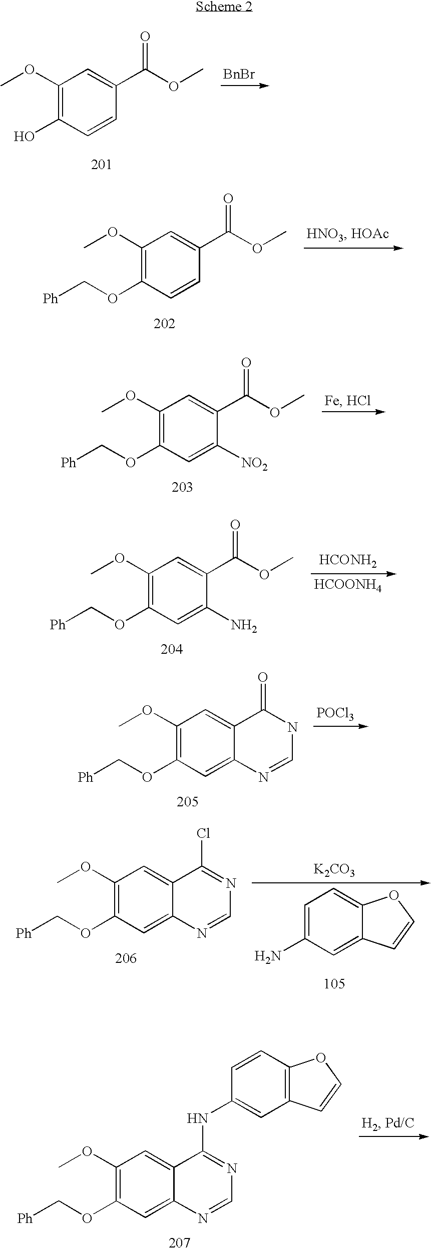

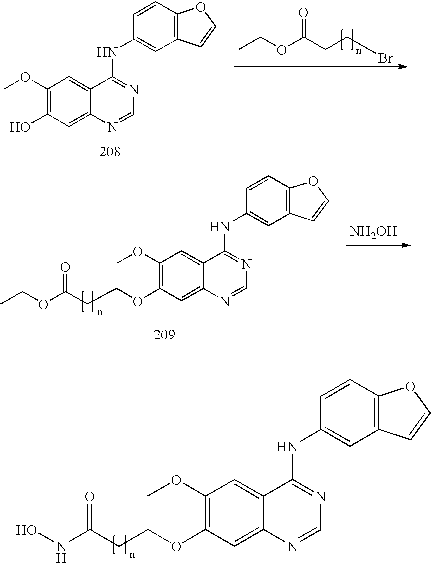

### Example 1

### Preparation of 7-(4-(benzofuran-5-ylamino)-7-methoxyquinazolin-6-yloxy)-N-hydroxyheptanamide (Compound 2)

### Step 1a. 2-Bromo-1-fluoro-4-nitrobenzene (Compound 102)

To a sulfuric acid (50 ml) solution of compound 101 (8.75 g, 500 mmol) was added 68% HNO₃ (4 mL) in such a way that the temperature of the reaction was maintained below 40° C. After the addition, the mixture was stirred at 20° C. for 1 h. The mixture was diluted with 300 mL of ice-water and filtered. The collected solid was recrystallized from petroleum ester to yield the title compound 102 as a white solid (8.06 g, 73.3%): ¹H NMR (DMSO-d₆); δ 8.6 (dd, 1H), 8.3 (m, 1H), 7.7 (t, 1H).

### Step 1b. ((2-Fluoro-5-nitrophenyl)ethynyl)trimethylsilane (Compound 103)

A mixture of compound 102 (2.5 g, 11.4 mmol), triphenylphosphine (0.114 g, 0.44 mmol), palladium (II) chloride (0.045 g, 0.26 mmol) and triethylamine (28 ml) was stirred and heated to 100° C. under nitrogen for 16 hours. The mixture was cooled to room temperature and the precipitate was filtered. The solid was washed with triethylamine and the combined filtrate was evaporated to leave a dark brown oil which was distilled out at 120° C. under reduced pressure to gave compound 103 as a brown yellow solid (1.708 g, 63%): LCMS: 238 [M+1]⁺.

### Step 1c. 5-Nitrobenzofuran (Compound 104)

A mixture of compound 103 (7.30 g, 30.8 mmol), sodium acetate (10.1 g, 123 mmol) and N,N-dimethylformamide (70 mL) was stirred and heated to 100° C. for 16 hours. The precipitate was filtered and washed with N,N-dimethylformamide. The combined filtrate was evaporated to leave a residue which was purified through a short silica gel column (eluant: ethyl acetate/petroleum ether=1/10) to provide the title compound 104 as a brown solid (3.0 g, 60%).

### Step 1d. Benzofuran-5-amine (Compound 105)

A mixture of compound 104 (1.89 g, 11.63 mmol), iron powder (6.5 g, 116 mmol), 36.5% HCl (1 ml), ethanol (30 mL) and water (6 mL) was stirred and heated to 100° C. for 3 h. The precipitate was filtered and washed with ethanol. The combined filtrate was evaporated to leave a residue which was dissolved in dichloromethane (50 mL). The organic layer was washed with aqueous NaHCO₃ solution (20 mL×2) and brine (20 mL×1) and dried over MgSO₄, filtered and evaporated to give the title compound 105 as a brown solid (0.8 g, 51%): LC-MS: 134 [M+1]⁺; ¹H NMR (DMSO-d₆): δ4.8 (s, 2H), 6.57 (m, 1H), 6.67 (m, 1H) 6.69 (m, 1H) 7.21 (d, J=9.3 Hz, 1H) 7.74 (d, J=2.4 Hz, 1H).

### Step 1e. 6,7-Dimethoxyquinazolin-4(3H)-one (Compound 107)

A mixture of compound 106 (2.1 g, 10 mmol), ammonium formate (0.63 g, 10 mmol) and formamide (7 mL) was stirred and heated to 190˜200° C. for 2 hours. The mixture was cooled to room temperature and the resulting precipitate was isolated, washed with water and dried to provide the title compound 107 as a brown solid (1.8 g, 84.7%): LCMS: 207 [M+1]; ¹H NMR (DMSO-d₆); δ 3.87 (s, 3H), 3.89 (s, 3H), 7.12 (s, 1H), 7.43 (s, 1H), 7.97 (s, 1H), 12.08 (bs, 1H).

### Step 1f. 6-Hydroxy-7-methoxyquinazolin-4(3H)-one methanesulfonate (Compound 108)

Compound 107 (10.3 g, 50 mmol) was added portionwise to a stirred methanesulphonic acid (68 mL). L-Methionone (8.6 g, 57.5 mmol) was then added and the mixture was heated to 150˜160° C. for 5 hours. The mixture was cooled to room temperature and poured onto a mixture of ice and water (250 mL). The mixture was neutralized by the addition of aqueous sodium hydroxide solution (40%). The resulting precipitate was isolated, washed with water and dried to yield title compound 108 as a grey solid (10 g, crude): LCMS: 193 [M+1]⁺, ¹H NMR (DMSO-d₆); δ 2.99 (s, 3H), 3.88 (s, 3H), 7.08 (s, 1H), 7.36 (s, 1H), 7.89 (s, 1H), 9.83 (bs, 1H), 11.86 (bs, 1H).

### Step 1g. 7-Methoxy-4-oxo-3,4-dihydroquinazolin-6-yl acetate (Compound 109)

A mixture of compound 108 (10 g, crude), acetic anhydride (100 mL) and pyridine (8 mL) was stirred and heated to reflux for 3 hours. The mixture was cooled to room temperature and poured into a mixture of ice and water (250 mL). The resulting precipitate was isolated and dried to yield the title product 109 as a grey solid (5.8 g, 50% two step overall yield): LCMS: 235 [M+1]; ¹H NMR (CDCl₃): δ 2.27 (s, 3H), 3.89 (s, 3H), 7.28 (s, 1H), 7.72 (s, 1H), 8.08 (d, J=6.0 Hz, 1H), 12.20 (bs, 1H).

### Step 1h. 4-Chloro-7-methoxyquinazolin-6-yl acetate (Compound 110)

A mixture of compound 109 (2.0 g, 8.5 mmol) and phosphoryl trichloride (20 mL) was stirred and heated to reflux for 3 hours. When a clear solution was obtained, the excessive phosphoryl trichloride was removed under reduced pressure. The residue was dissolved in dichloromethane (50 mL) and the organic layer was washed with aqueous NaHCO₃ solution (20 mL×2) and brine (20 mL×1) and dried over MgSO₄, filtered and evaporated to give the title product 110 as a yellow solid (1.4 g, 65%): LCMS: 253 [M+1]⁻.

### Step 1i. 4-(Benzofuran-5-ylamino)-7-methoxyquinazolin-6-ol (Compound 111)

A mixture of compound 110 (0.151 g, 0.6 mmol) and 105 (0.20 g, 1.504 mmol) in isopropanol (2 mL) was stirred and heated to reflux over night. The mixture was cooled to room temperature and filtered to give the title product 111 as a white solid (0.169 g, 92%): LCMS: 308 [M+1]⁺.

### Step 1j. Ethyl 7-(4-(benzofuran-5-ylamino)-7-methoxyquinazolin-6-yloxy)heptanoate (Compound 112-2)

A mixture of compound 111 (0.169 g, 0.55 mmol), ethyl 7-bromoheptanoate (0.13 g, 0.55 mmol) and potassium carbonate (0.38 g, 2.75 mmol) in N,N-dimethylformamide (5 mL) was stirred at 60° C. for 3 hour. The precipitate was filtered and the filtrate was poured into water. The resulting precipitate was filtered, washed with ethyl acetate and dried to give the title compound 112-2 as a grey solid (0.207 g, 81%).

### Step 1k. 7-(4-(Benzofuran-5-ylamino)-7-methoxyquinazolin-6-yloxy)-N-hydroxyheptanamide (Compound 2)

To a stirred solution of hydroxylamine hydrochloride (4.67 g, 67 mmol) in methanol (24 mL) at 0° C. was added a solution of potassium hydroxide (5.61 g, 100 mmol) in methanol (14 mL). After addition, the mixture was stirred for 30 minutes at 0° C., and was allowed to stand at low temperature. The resulting precipitate was isolated, and the solution was prepared to give free hydroxylamine. The freshly prepared hydroxylamine solution (2.5 mL) was placed in 10 mL flask. Compound 112-2 (207 mg, 0.45 mmol) was added to this solution and stirred at 25° C. for 0.5 hour. The mixture was neutralized with acetic acid, and the resulting precipitate was isolated, washed with water, and dried to give the title compound 2 as a white solid (97 mg, 48%): mp 191˜195° C., LCMS: 451 [M+1]; ¹H NMR (DMSO-d₆): δ 1.33 (m, 2H), 1.43 (m, 2H), 1.51 (m, 2H), 1.82 (m, 2H), 1.94 (m, 2H), 3.90 (s, 3H), 4.15 (m, 2H), 7.03 (m, 1H), 7.22 (s, 1H, 7.50 (m, 1H, 7.70 (d, J=2.7 Hz, 1H, 7.90 (d, J=2.1 Hz, 1H, 8.03 (s, 1H), 8.06 (d, J=2.4 Hz, 1H), 8.65 (s, 1H), 8.71 (s, 1H), 10.33 (s, 1H), 10.84 (s, 1H).

### Example 2

### Preparation of 7-(4-(benzofuran-5-ylamino)-6-methoxyquinazolin-7-yloxy)-N-hydroxyheptanamide (Compound 6)

### Step 2a. Methyl 4-(benzyloxy)-3-methoxybenzoate (Compound 202)

To a mixture of compound 201 (18.2 g, 0.1 mol), potassium carbonate (34.55 g, 0.25 mol) in N,N-dimethylformamide was added benzylbromide (14.5 ml, 0.105 mol) dropwise. The reaction was then heated to 60° C. and stirred for 2 hours. The mixture was cooled to room temperature and was filtered. The filtrate was concentrated and the residue was dissolved in ethyl acetate 500 mL. The organic layer was washed with water and brine (100 mL), dried over MgSO₄, filtered and concentrated to give the title compound 202 as a white solid (26 g, 95%): LCMS: 273 [M+1]⁺.

### Step 2b. Methyl 4-(benzyloxy)-5-methoxy-2-nitrobenzoate (Compound 203)

A mixture of HNO₃ (45 mL, 0.963 mol) and HOAc (45 mL) was placed in an ice-bath and stirred. Compound 202 (10.3 g, 50 mmol) in 200 ml HOAc was added dropwise. After addition, the reaction mixture was stirred at −10° C. for 20 min. The mixture was poured onto a mixture of ice and water (250 mL) and was neutralized by the addition of aqueous sodium hydroxide solution (40%). The precipitate was isolated by filtration, washed with water and dried to yield title compound 203 as a grey solid (30 g, 98%): LCMS: 318 [M+1]⁺.

### Step 2c. Methyl 2-amino-4-(benzyloxy)-5-methoxybenzoate (Compound 204)

A mixture of compound 203 (10 g, crude), iron powder (54 g, 0.96 mol), ethanol (100 mL), and H₂O (20 mL) was stirred and heated to reflux for 3 hours. The mixture was cooled to room temperature and neutralized with aqueous sodium hydroxide (10%). The reaction was filtered and the filtrate was concentrated to give a residue which was extracted with dichloromethane (200 mL×2). The combined organic layer was washed with brine and dried over MgSO₄, filtered and concentrated to yield the title compound 204 as a grey solid (14.5 g, 85%): LCMS: 288 [M+1]⁺.

### Step 2d. 7-(Benzyloxy)-6-methoxyquinazolin-4(3H)-one (Compound 205)

A mixture of compound 204 (7.5 g, 25 mmol), ammonium formate (1.1 g, 22.4 mmol) and formamide (60 mL) was stirred and heated at 180˜190° C. (oil bath temperature) for 2 hours. Then the mixture was cooled to room temperature and the resulting precipitate was isolated, washed with water and dried to give the title compound 205 as a brown solid (6.5 g, 95%): LCMS: 283 [M+1]⁺.

### Step 2e. 7-(Benzyloxy)-4-chloro-6-methoxyquinazoline (Compound 206)

A mixture of compound 205 (6.5 g, 8.5 mmol) and phosphoryl trichloride (40 mL) was stirred and heated to reflux for 3 hours. When a clear solution was obtained, the excessive phosphoryl trichloride was removed under reduced pressure. The residue was dissolved in dichloromethane (200 mL) and the organic layer was washed with aqueous NaHCO₃ solution (100 mL×3) and brine (100 mL×1) and dried over MgSO₄, filtered and evaporated to give the title compound 206 as a yellow solid (1.4 g, 65%): LCMS: 301 [M+1]⁺.

### Step 2f. N-(Benzofuran-5-yl)-7-(benzyloxy)-6-methoxyquinazolin-4-amine (Compound 207)

A mixture of compound 206 (0.5 g, 1.5 mmol) and compound 105 (0.2 g, 1.5 mmol) in isopropanol (5 mL) was stirred and heated to reflux for 3 hours. The mixture was cooled to room temperature and filtered to give the title product 207 as a white solid (0.546 g, 91%): LCMS: 398 [M+1]⁺.

### Step 2g. 4-(Benzofuran-5-ylamino)-6-methoxyquinazolin-7-ol (Compound 208)

A mixture of compound 207 (0.51 g, 1.3 mmol) and Pd/C (0.2 g) in methanol (6 mL) was stirred at room temperature for 4 hour. The precipitate was isolated and dried to give the title compound 208 as a grey solid (0.4 g, 100%): LCMS: 308 [M+1]⁺.

### Step 2h. Ethyl 7-(4-(benzofuran-5-ylamino)-6-methoxyquinazolin-7-yloxy)heptanoate (Compound 209-6)

A mixture of compound 208 (0.4 g, 1.3 mmol), ethyl 7-bromoheptanoate (0.31 g, 1.3 mmol) and potassium carbonate (0.89 g) in N,N-dimethylformamide (15 mL) was stirred at 60° C. for 3 hour. The precipitate was isolated by filtration and the filtrate was poured to water. The resulting solid was filtered, washed with ethyl acetate and dried to give the title compound 209-6 as a grey solid (0.6 g, 100%): LC-MS: 464 [M+1]⁺.

### Step 2i. 7-(4-(Benzofuran-5-ylamino)-6-methoxyquinazolin-7-yloxy)-N-hydroxyheptanamide (Compound 6)

The title compound 6 was prepared as a white solid (96 mg, 16%) from compound 209-6 (600 mg, 1.3 mmol) and freshly prepared NH₂OH/MeOH (7.3 mL, 13 mmol) using a procedure similar to that described for compound 2 (Example 1): mp 214˜217° C., LC-MS: 451 [M+1]⁺; ¹H NMR (DMSO-d₆): δ1.34 (m, 2H), 1.45 (m, 2H), 1.53 (m, 2H), 1.79 (m, 2H), 1.97 (m, 2H), 3.97 (s, 3H, 4.97 (m, 2H), 7.00 (m, 1H, 7.16 (s, 1H), 7.58 (m, 1H, 7.62 (d, J=9.0 Hz, 1H), 7.90 (s, 1H), 8.00 (d, J=2.1 Hz, 1H), 8.07 (d, J=1.2 Hz, 1H), 8.41 (s, 1H), 8.67 (s, 1H), 9.53 (s, 1H), 10.34 (s, 1H).

### Biological Assays:

As stated hereinbefore the derivatives defined in the present invention possess anti-proliferation activity. These properties may be assessed, for example, using one or more of the procedures set out below:

(a) An In Vitro Assay which Determines the Ability of a Test Compound to Inhibit a Receptor Tyrosine Kinase.

The ability of compounds to inhibit receptor kinase (VEGFR2 and PDGFR-beta) activity was assayed using HTScan™ Receptor Kinase Assay Kits (Cell Signaling Technologies, Danvers, Mass.). VEGFR2 tyrosine kinase was produced using a baculovirus expression system from a construct containing a human VEGFR2 cDNA kinase domain (Asp805-Val356) (GenBank accession No. AF035121) fragment amino-terminally fused to a GST-HIS6-Thrombin cleavage site. PDGFR-beta tyrosine kinase was produced using a baculovirus expression system from a construct containing a human PDGFR-beta c-DNA (GenBank Accession No. NM_{—}002609) fragment (Arg561-Leu1106) amino-terminally fused to a GST-HIS6-Thrombin cleavage site. The proteins were purified by one-step affinity chromatography using glutathione-agarose. An anti-phosphotyrosine monoclonal antibody, P-Tyr-100, was used to detect phosphorylation of biotinylated substrate peptides (VEGFR2, Biotin-Gastrin Precursor (Tyr87); PDGFR-β, Biotinylated-FLT3 (Tyr589)). Enzymatic activity was tested in 60 mM HEPES, 5 mM MgCl2 5 mM MnCl2 200 μM ATP, 1.25 mM DTT, 3 μM Na3VO4, 1.5 mM peptide, and 50 ng EGF Receptor Kinase. Bound antibody was detected using the DELFIA system (PerkinElmer, Wellesley, Mass.) consisting of DELFIA® Europium-labeled Anti-mouse IgG (PerkinElmer, #AD0124), DELFIA® Enhancement Solution (PerkinElmer, #1244-105), and a DELFIA® Streptavidin coated, 96-well Plate (PerkinElmer, AAAND-0005). Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm.

Test compounds were dissolved in dimethylsulphoxide (DMSO) to give a 20 mM working stock concentration. Each assay was setup as follows: Added 100 μl of 10 mM ATP to 1.25 ml 6 mM substrate peptide. Diluted the mixture with dH₂0 to 2.5 ml to make 2×ATP/substrate cocktail ([ATP]=400 mM, [substrate]=3 mM). Immediately transfer enzyme from −80° C. to ice. Allowed enzyme to thaw on ice. Microcentrifuged briefly at 4° C. to bring liquid to the bottom of the vial. Returned immediately to ice. Added 10 μl of DTT (1.25 mM) to 2.5 ml of 4×HTScan™ Tyrosine Kinase Buffer (240 mM HEPES pH 7.5, 20 mM MgCl₂, 20 mM MnCl, 12 mM NaVO₃) to make DTT/Kinase buffer. Transfer 1.25 ml of DTT/Kinase buffer to enzyme tube to make 4× reaction cocktail ([enzyme]=4 ng/μL in 4× reaction cocktail). Incubated 12.5 μl of the 4× reaction cocktail with 12.5 μl/well of prediluted compound of interest (usually around 10 μM) for 5 minutes at room temperature. Added 25 μl of 2×ATP/substrate cocktail to 25 μl/well preincubated reaction cocktail/compound. Incubated reaction plate at room temperature for 30 minutes. Added 50 μl/well Stop Buffer (50 mM EDTA, pH 8) to stop the reaction. Transferred 25 μl of each reaction and 75 μl dH₂O/well to a 96-well streptavidin-coated plate and incubated at room temperature for 60 minutes. Washed three times with 200 μl/well PBS/T (PBS, 0.05% Tween-20). Diluted primary antibody, Phospho-Tyrosine mAb (P-Tyr-100), 1:1000 in PBS/T with 1% bovine serum albumin (BSA). Added 100 μl/well primary antibody. Incubated at room temperature for 60 minutes. Washed three times with 200 μl/well PBS/T. Diluted Europium labeled anti-mouse IgG 1:500 in PBS/T with 1% BSA. Added 100 μl/well diluted antibody. Incubated at room temperature for 30 minutes. Washed five times with 200 μl/well PBS/T. Added 100 μl/well DELFIA® Enhancement Solution. Incubated at room temperature for 5 minutes. Detected 615 nm fluorescence emission with appropriate Time-Resolved Plate Reader.

(b) An In Vitro Assay which Determines the Ability of a Test Compound to Inhibit HDAC Enzymatic Activity.

HDAC inhibitors were screened using an HDAC fluorimetric assay kit (AK-500, Biomol, Plymouth Meeting, Pa.). Test compounds were dissolved in dimethylsulphoxide (DMSO) to give a 20 mM working stock concentration. Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm. Each assay was setup as follows: Defrosted all kit components and kept on ice until use. Diluted HeLa nuclear extract 1:29 in Assay Buffer (50 mM Tris/Cl, pH 8.0, 137 mM NaCl, 2.7 mM KCl, 1 mM MgCl2). Prepared dilutions of Trichostatin A (TSA, positive control) and tested compounds in assay buffer (5× of final concentration). Diluted Fluor de Lys™ Substrate in assay buffer to 100 uM (50 fold=2× final). Diluted Fluor de Lys™ developer concentrate 20-fold (e.g. 50 μl plus 950 μl Assay Buffer) in cold assay buffer. Second, diluted the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Added Assay buffer, diluted trichostatin A or test inhibitor to appropriate wells of the microtiter plate. Added diluted HeLa extract or other HDAC sample to all wells except for negative controls. Allowed diluted Fluor de Lys™ Substrate and the samples in the microtiter plate to equilibrate to assay temperature (e.g. 25 or 37° C. Initiated HDAC reactions by adding diluted substrate (25 μl) to each well and mixing thoroughly. Allowed HDAC reactions to proceed for 1 hour and then stopped them by addition of Fluor de Lys™ Developer (50 μl). Incubated plate at room temperature (25° C.) for 10-15 min. Read samples in a microtiter-plate reading fluorimeter capable of excitation at a wavelength in the range 350-380 nm and detection of emitted light in the range 440-460 nm.

The following TABLE 12-B lists compounds representative of the invention and their activity in HDAC, VEGFR2 and PDGFR assays. In these assays, the following grading was used: I≧10 μM, 10 μM>II>1 μM, 1 μM>III>0.1 μM, and IV≦0.1 μM for IC₅₀.

TABLE 12-B

| Compound | | | HER2/ | |
|---|---|---|---|---|
| No. | HDAC | EGFR | ErbB | VEGFR2 |
| 2 | IV | IV | IV | II |
| 6 | IV | | | II |
| 9 | III | | | |
| 10 | IV | | | |
| 11 | IV | | | |

TABLE 13-A
SECTION 13:

| | |
|---|---|
| | (XVI) |
| 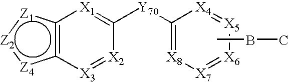 | |

| Compound # | Structure |
|---|---|
| 1 | 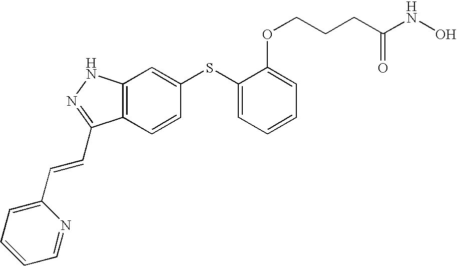 |
| 2 | 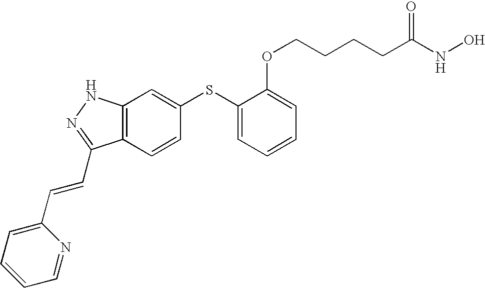 |
| 3 | 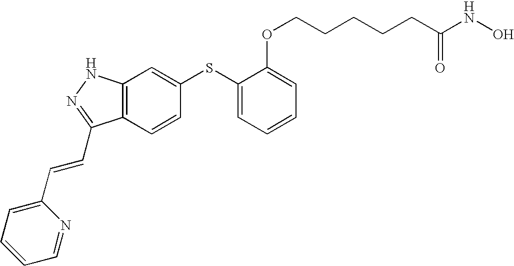 |
| 4 | 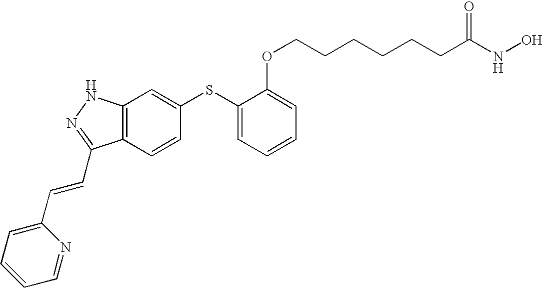 |
| 5 | 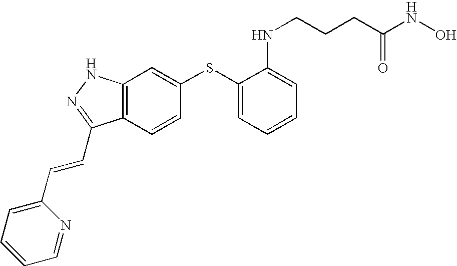 |
| 6 | 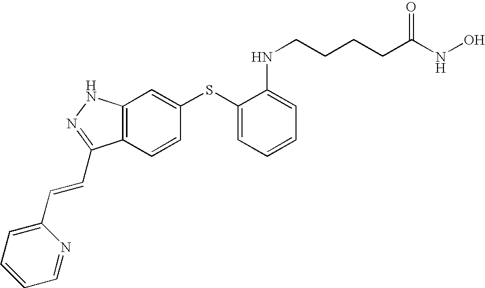 |
| 7 | 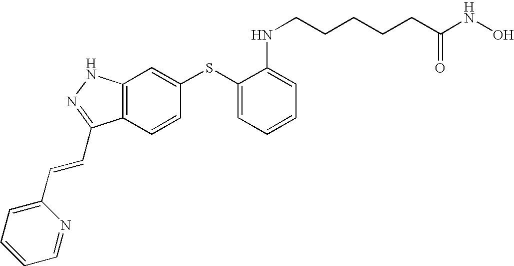 |
| 8 | 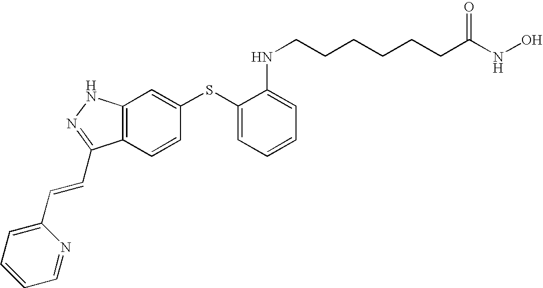 |
| 9 | 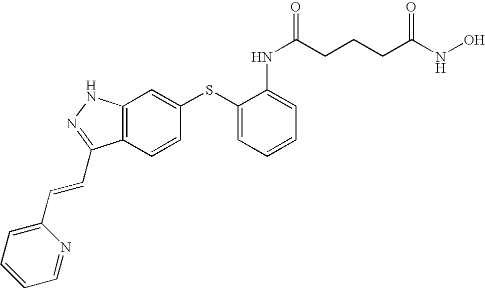 |
| 10 | 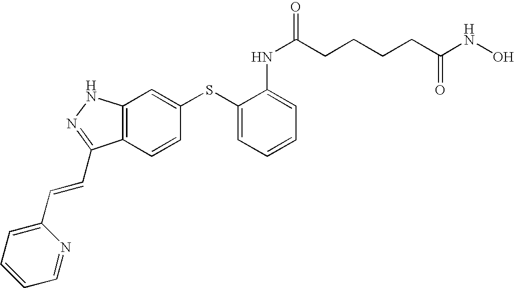 |
| 11 | 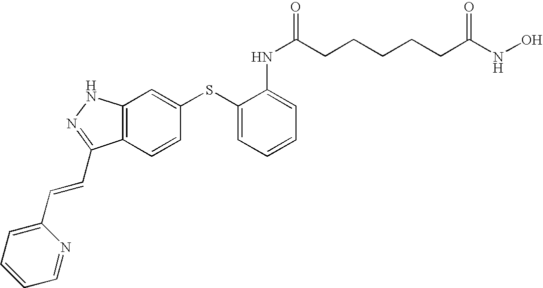 |
| 12 | 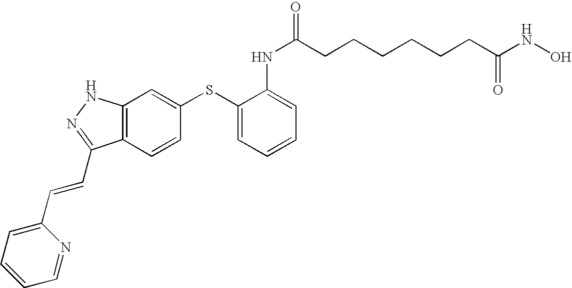 |
| 13 | 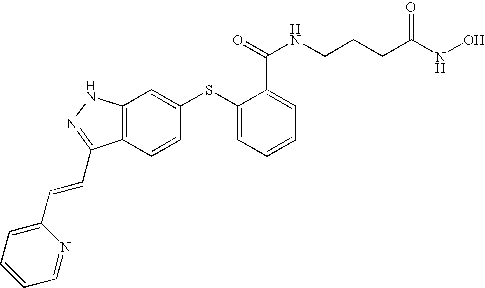 |
| 14 | 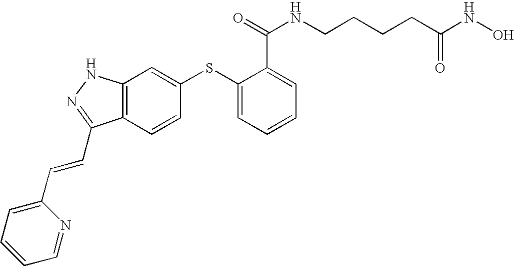 |
| 15 | 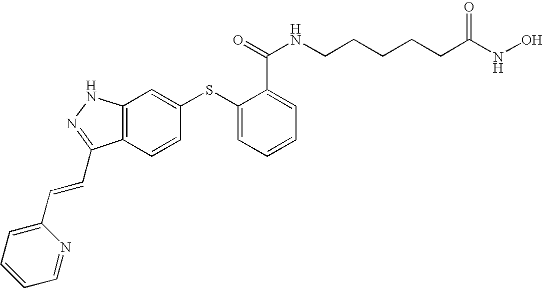 |
| 16 | 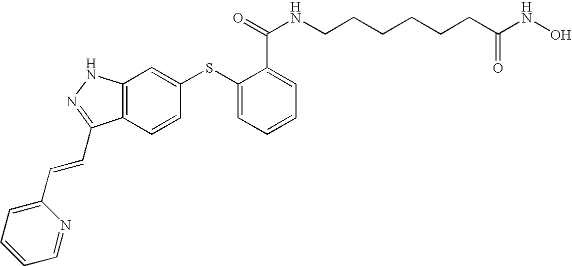 |
| 17 | 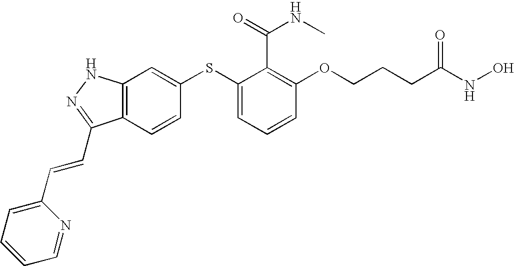 |
| 18 | 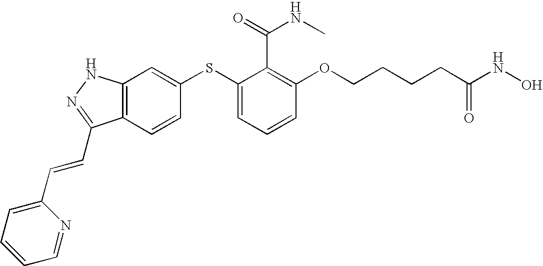 |
| 19 | 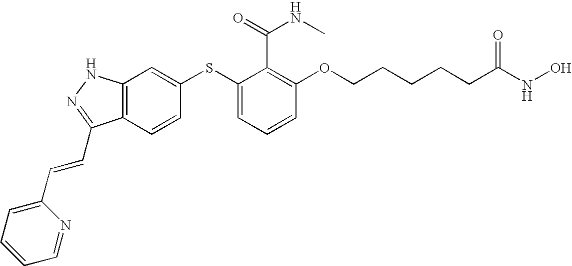 |
| 20 | 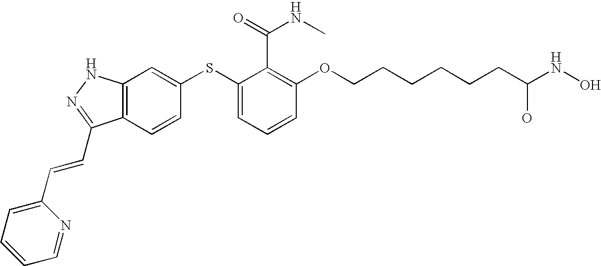 |

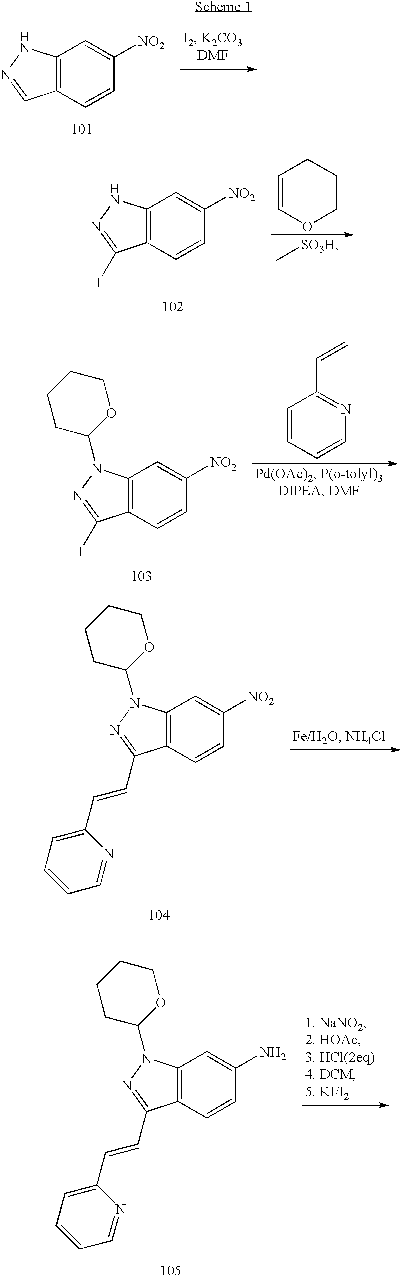

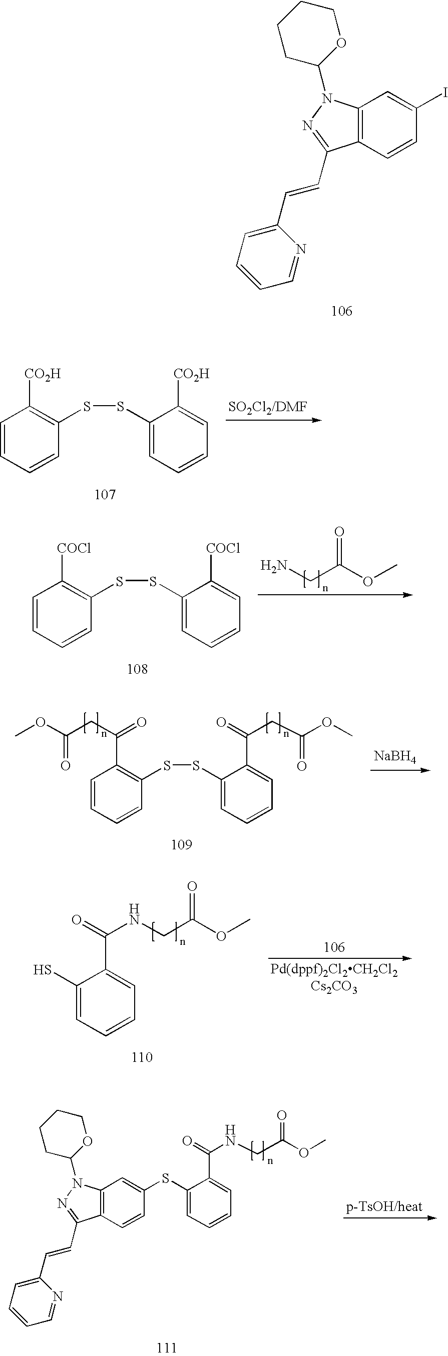

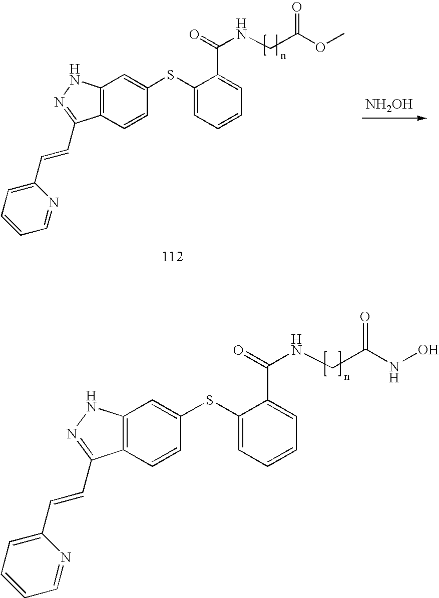

### Example 1

### Preparation of N-(5-(hydroxycarbamoyl)pentyl)-2-(3-((E)-2-(pyridin-2-yl)vinyl)-1H-indazol-6-ylthio)benzamide (Compound 15)

### Step 1a. 3-Iodo-6-nitro-1H-indazole (Compound 102)

To a solution of 6-nitroindazole (23 g, 141 mmol) in DMF (100 mL) was added potassium carbonate (39 g, 282 mmol) while maintain reaction temperature to be ≦30° C. A solution of iodine (62 g, 244 mmol) pre-dissolved in DMF (50 mL) was added over a period of 2 h while the reaction temperature was maintained ≦35° C. The reaction mixture is stirred at 25° C. After reaction complete, the mixture was then added a solution of sodium thiosulfate (34 g, 215 mmol) and potassium carbonate (0.23 g) pre-dissolved in water (228 ml) while the solution temperature is maintained ≦30° C. The mixture is agitated for 20 min at room temperature. Water (340 mL) is added which precipitates solids and the slurry is agitated for 20 min at room temperature. The solid are filtered, washed with water (2×50 mL), and dried in a vacuum oven for 12 h (50° C. and 25 mmHg) to provide the title compound 102 as a yellow solid (39 g, 95% yield): LCMS: 289 [M+1]; ¹H NMR (DMSO-d₆): δ14.21 (s, 1H), 8.47 (s, 1H), 7.97-8.01 (m, 1H), 7.67-7.70 (d, J=8.7 Hz, 1H).

### Step 1b. 3-Iodo-6-nitro-1-(tetrahydro-2H-pyran-2-yl)-1H-indazole (Compound 103)

To a solution of compound 102 (22 g, 76.2 mmol) in methylene chloride (90 g) and THF (60 g) was added methane sulfuric acid (1.0 g, 10.4 mmol) carefully. To the mixture was then added a solution of DHP (17 g, 202 mmol) in methylene chloride (30 g) over a period of 1 h while the reaction temperature was maintained at <25° C. The mixture was agitated at 25° C. for 5 h (until the reaction was completed by HPLC). The mixture was then carefully added to an aqueous solution of 10% NaHCO₃ (11.1 g of NaHCO₃ dissolved in 111 g water) while the solution temperature was maintained at room temperature. The mixture was agitated for 1 h at 25° C. and the layers separated. The organic layer was washed with an aqueous solution of 10% NaCl (120 g) and layers separated. The organic layer was concentrated at 50° C. under reduced pressure to remove the remaining solvents. The resulting slurry was diluted with acetonitrile (50 g) and was agitated for 2 h at −5° C. The slurry was filtered, and the solids were rinsed with cold acetonitrile (20 g). The solids were dried at room temperature under reduced pressure to provide compound 103 (24 g, 85% yield): ¹H NMR (DMSO-d₆). δ 8.79 (s, 1H), 8.03-8.07 (m, 1H), 7.69-7.72 (d, J=54 Hz, 1H), 6.11-6.15 (m, 1H), 3.82-3.88 (m, 2H), 2.34-2.38 (m, 1H), 2.01-2.08 (m, 2H), 1.56-1.76 (m, 3H).

### Step 1c. (E)-6-nitro-3-(2-(pyridin-2-yl)vinyl)-1-(tetrahydro-2H-pyran-2-yl)-1H-indazole (Compound 104)

Compound 103 (24.4 g, 65.4 mmol) was added to a solution of 2-vinyl pyridine (9.82 g, 93.4 mmol), N,N-diisopropylethylamine (16.2 g, 125 mmol) and tri-o-tolylphosphine (1.72 g, 5.65 mmol) in DMF (163 g). PdCl₂ (0.38 g, 2.1 mmol) was added and the mixture was agitated for 12 h at 100° C. (until the reaction was completed by HPLC). The mixture was then cooled to 45° C. and isopropanol (80 g) was added. The mixture was agitated for 30 min at 45° C., diluted with water (400 mL), and the mixture was agitated at 25° C. for 1 h. The resulting slurry was filtered, rinsed with water (25 mL), and the solids were combined with isopropanol (100 g). The mixture was agitated for 30 min at 55° C., then for 30 min at 10° C., filtered, and the solids were washed with cold isopropanol (2×10 mL). The solids were dried in a vacuum oven for 12 h (50° C. and 25 mmHg) to provide compound 104 (22 g, 96% yield): LCMS: 351 [M+1].

### Step 1d. (E)-3-(2-(pyridin-2-yl)vinyl)-1-(tetrahydro-2H-pyran-2-yl)-1H-indazol-6-amine (Compound 105)

Compound 104 (22.0 g, 62.9 mmol) was dissolved in an aqueous solution of ammonium chloride (25.5 g of NH₄Cl in 80 g water) and ethanol (120 mL). Iron powder (14.1 g, 252 mmol) was added and the mixture was agitated for 2 h at 50° C. (until the reaction was completed by HPLC). The mixture was then cooled to 22° C. and THF (300 mL) was added. The mixture was agitated for 1 h at room temperature, and filtered through diatomaceous earth. The cake was rinsed with THF (60 mL), and the filtrate was concentrated at 50° C. under reduced pressure to a volume of ca. 50 ml. The concentrate was cooled to room temperature, diluted with water (200 mL), and agitated at room temperature for 1 h. The mixture was filtered, rinsed with hexane (20 mL), dried in a vacuum oven for 12 h (50° C. and 25 mmHg) to provide compound 105 (15 g, 75% yield): LCMS: 321 [M+1]⁺; ¹H NMR (DMSO-d₆): δ 8.57-8.59 (m, 1H), 7.76-7.81 (m, 3H), 7.63-7.66 (d, J=7.8 Hz, 1H), 7.42-7.48 (d, J=16.5 Hz, 1H), 7.23-7.28 (m, 1H), 6.63-6.66 (m, 2H), 5.56-5.60 (m, 1H), 5.47 (s, 2H), 3.88-3.92 (d, J=10.8 Hz, 1H), 3.64-3.72 (m, 1H), 2.30-2.50 (m, 1H). 1.91-2.07 (m, 2H), 1.53-1.59 (m, 3H).

### Step 1e. (E)-6-iodo-3-(2-(pyridin-2-yl)vinyl)-1-(tetrahydro-2H-pyran-2-yl)-1H-indazole (Compound 106)

Compound 105 (10.0 g, 31.3 mmol) dissolved in acetic acid (65 mL) was added over 1 h to a solution of sodium nitrite (3.5 g, 50.7 mmol) dissolved in water (30 ml) at 0° C. The mixture was stirred for 1 h at 0° C., and a solution of HCl (5.6 mL diluted in 10 mL of water) at 0° C. was added over 10 min. The mixture was stirred for 1 h at 0° C. The formation of the diazolium salts was monitored by HPLC. Methylene chloride (40 mL) at 0° C. was added over 5 min to the diazonium salt solution at 0° C., and a solution of potassium iodide (10.62 g, 63.9 mmol) and iodine (3.96 g, 15.6 mmol) dissolved in water (30 mL) at 0° C. was added over 1 h. The reaction mixture was agitated for 2 h at 0° C. (until completed by HPLC). The mixture was then poured into a solution of 20% aqueous sodium hydrogen sulfide (20 g sodium thiosulfate in 100 mL water) and methylene chloride (40 mL) at 0° C., agitated, and the layers separated. The aqueous layer was extracted with methylene chloride (2×40 ml) at 0° C. and combined. A solution of 3 M aqueous sodium hydroxide (170 mL) at 0° C. was added over 10 min to the combined organic layers until the aqueous phase was basic (Ph=9-12). The phase separation was not clear due to the formation of an emulsion. A solution of 28% aqueous ammonium hydroxide (10 mL) and water (20 mL) was added, and the mixture was agitated for 30 min at 10° C., and allowed to settle for 12 h to afford a clear phase separation. The layers were separated and the aqueous layer was extracted with methylene chloride (2×60 mL). The combined organic layers were concentrated and separated by a glass fritted column containing silica gel with methylene chloride to provide compound 106 (8.8 g. 65% yield): LCMS: 432 [M+1]⁺; ¹H NMR (DMSO-d₆): δ8.60-8.62 (d, J=4.8 Hz, 1H), 8.26 (s, 1H), 8.01-8.03 (d, J=8.4 Hz, 1H), 7.88-7.93 (d, J=16.5 Hz, 1H), 7.79-7.82 (m, 1H), 7.68-7.71 (d, J=7.8 Hz, 2H), 7.55-7.61 (m, 2H), 7.29-7.31 (m, 1H), 5.91-5.93 (m, 1H), 3.90-4.00 (m, 2H), 2.49-2.59 (m, 1H), 2.08-2.20 (m, 2H), 1.70-1.86 (m, 3H).

### Step 1f. 2,2-Dithiosalicylic acid dichloride (Compound 108)

2,2′-dithiosalicylic acid 107 (3.22 g, 10.5 mmol) was dissolved in toluene (30 mL) and thionyl chloride (2 mL) and DMF (0.2 mL) were added. The mixture was stirred at 80° C. overnight. Solvents were evaporated to obtain compound 108 as a yellow solid (3.2 g, 89% yield).

### Step 1g. 2,2′-Dithio-N-(ethyl hexanoate)-yl-benzamide (Compound 109-15)

KOH (878 mg, 15.66 mmol) was added to a solution of methyl 5-aminohexanoate hydrochloride in methanol (5 mL). The mixture was stirred at room temperature for 10 min. and the mixture was then concentrated. Compound 108 (1.41 g, 4.12 mmol) dissolved in THF (5 mL) was added at 0° C. The mixture was stirred for 1 h. After solvent THF was evaporated, ethyl acetate (200 mL) was added. The organic layer was washed with water and brine, dried over anhydrous Na₂SO₄, and evaporated to obtain 109-15 as a white solid (1.23 g, 53% yield): LCMS: 561 [M+1]⁺; ¹H NMR (DMSO-d₆): δ 1.30-1.38 (m, 4H), 1.48-1.60 (m, 8H), 2.29 (t, J=7.5 Hz, 4H), 3.20-3.26 (m, 4H), 3.30 (s, 6H), 7.24-7.27 (m, 2H), 7.29-7.44 (m, 2H), 7.57-7.62 (m, 4H), 8.57 (t, J=6 Hz, 2H).

### Step 1h. Methyl 6-(2-mercaptobenzamido)hexanoate (Compound 110-15)

Compound 109-15 (831 mg, 1.48 mmol) was dissolvent in ethanol (10 mL) and cooled to 0° C. Sodium borohydride (130 mg, 2.96 mmol) was added in portions, and the mixture was stirred for 1 h. Hydrochloric acid (3 M, 10 mL) was added to the mixture and the mixture was extracted with ethyl acetate (80 mL×3). The organic layer was washed with brine, dried over anhydrous Na₂SO₄ and evaporated to obtain compound 110-15 which was used in next step without purification (0.49 g, 59% yield): LCMS: 282 [M+1]⁺.

### Step 1i. Methyl 6-(2-(1-(tetrahydro-2H-pyran-2-yl)-3-((E)-2-(pyridin-2-yl)vinyl)-1H-indazol-6-ylthio)benzamido)hexanoate (Compound 111-15)

Compound 106 (600 mg, 1.40 mmol) in DMF (6 mL) was added to a mixture of [1,1′-bis(diphenyl-phosphino)ferrocene]dichloro-palladium(II) complex with dichloromethane (50 mg), and cesium carbonate (680 mg) in dichloromethane (50 mg). Compound 110-15 (490 mg, 1.74 mmol) was added and the mixture was stirred at 80° C. overnight. The mixture was cooled to room temperature and ethyl acetate (10 mL) was added and stirred for 20 min. Water (14 mL) was then added and the mixture was stirred for additional 40 min. The mixture was filtered and the solids were washed with water and ethyl acetate, dried to obtain compound 111-15 as a white solid (500 mg, 61% yield): LCMS: 585 [M+1]⁺; ¹H NMR (DMSO-d₆): δ 1.30-1.38 (m, 2H), 1.48-1.60 (m, 6H), 1.72-1.80 (m, 1H), 1.97-2.06 (m, 2H), 2.28 (t, J=7.5 Hz, 2H), 2.34-2.44 (m, 1H), 3.20-3.26 (m, 2H), 3.33 (s, 3H), 3.56-3.80 (m, 1H), 3.88-3.92 (m, 1H), 5.90-5.94 (m, 1H), 7.00-7.03 (m, 1H), 7.19-7.23 (m, 1H), 7.28-7.34 (m, 3H), 7.50-7.57 (m, 1H), 7.65 (m, 2H), 7.69 (d, J=7.8 Hz, 1H), 7.79-7.83 (m, 1H), 7.90-7.95 (m, 2H), 8.21 (d, J=8.1 Hz, 1H), 8.44 (t, J=5.4 Hz, 1H), 8.60-8.63 (m, 1H).

### Step 1j. Methyl 6-(2-(3-((E)-2-(pyridin-2-yl)vinyl)-1H-indazol-6-ylthio)benzamido)hexanoate (Compound 112-15)

Compound 111-15 (386 mg, 0.66 mmol), p-TsOH (630 mg,), methanol (6 mL) and water (1 mL) were combined and stirred for 1 h at 60° C. The mixture was concentrated under reduced pressure. This process was repeated for three times. Then the mixture was extracted for three times with ethyl acetate (60 mL). The organic layer was washed with water and brine, dried over anhydrous Na₂SO₄, evaporated to obtain a residue which was purified by column chromatography to yield compound 112-15 as a white solid (150 mg, 45% yield): LCMS: 501 [M+1]⁺; ¹H NMR (DMSO-d₆): δ 1.30-1.38 (m, 2H), 1.47-1.56 (m, 4H), 1.95 (t, J=6.9 Hz, 2H), 3.18-3.25 (m, 2H), 3.56 (s, 3H), 7.06-7.10 (m, 1H), 7.15-7.19 (m, 1H), 7.20-7.34 (m, 3H), 7.45-7.48 (m, 1H), 7.54-7.59 (m, 2H), 7.65-7.68 (m, 1H), 7.78-7.84 (m, 1H), 7.91-7.18 (m, 1H), 8.19 (d, J=8.1 Hz, 1H), 8.42 (t, J=5.4 Hz, 1H), 8.60-8.63 (m, 1H), 13.32 (s, 1H).

### Step 1k. N-(5-(Hydroxycarbamoyl)pentyl)-2-(3-((E)-2-(pyridin-2-yl)vinyl)-1H-indazol-6-ylthio)benzamide (Compound 15)

Preparation of the solution of hydroxylamine in methanol: hydroxylamine hydrochloride (4.67 g, 67 mmol) was dissolved in methanol (24 mL) to form solution A. Potassium hydroxide (5.61 g, 100 mmol) was dissolved in methanol (14 mL) to form solution B. Solution A was cooled to 0° C., and solution B was added into solution A dropwise. The mixture was stirred for 30 minutes at 0° C., and the precipitate was filtered to afford the solution of hydroxylamine in methanol.

To a flask containing compound 112-15 (150 mg, 0.28 mmol) was added a solution of hydroxylamine in methanol (4.0 mL). The mixture was stirred at room temperature for 30 min. It was then adjusted to pH 6-7 with acetic acid. The mixture was concentrated to give a residue which was taken into ethyl acetate (200 mL) and was washed with water, dried over anhydrous Na₂SO₄, and concentrated to afford compound 15 as a white solid (110 mg, 49% yield): LCMS: 502 [M+1]⁺; ¹H NMR (DMSO-d₆): δ 1.28-1.29 (m, 2H), 1.31-1.34 (m, 4H), 2.29 (t, J=7.5 Hz, 2H), 3.21-3.25 (m, 2H), 7.06-7.08 (m, 1H), 7.17-7.20 (m, 1H), 7.26-7.33 (m, 3H), 7.46-7.49 (m, 1H), 7.54-7.68 (m, 3H), 7.78-7.84 (m, 1H), 7.94 (d, J=16.2 Hz, 1H), 8.19 (d, J=8.7 Hz, 1H), 8.43 (t, J=5.4 Hz, 1H), 8.60-8.62 (m, 1H).

### Example 2

### Preparation of N-(6-(hydroxycarbamoyl)hexyl)-2-(3-((E)-2-(pyridin-2-yl)vinyl)-1H-indazol-6-ylthio)benzamide (Compound 16)

### Step 2a. 2,2′-Dithio-N-(ethyl heptanoate)-yl-benzamide (Compound 109-16)

The title compound 109-16 was prepared (3.42 g, 67%) from compound 108 (2.83 g, 8.24 mmol) and ethyl 7-aminoheptanoate hydrogen chloride (6.90 g, 32.96 mmol) using a procedure similar to that described for compound 109-15 (Example 1): LCMS: 617 [M+1]⁺.

### Step 2b. Ethyl 7-(2-mercaptobenzamido)heptanoate (Compound 110-16)

The title compound 110-16 was prepared (400 mg, 100%) from compound 109-16 (400 mg, 0.649 mmol) using a procedure similar to that described for compound 110-15 (Example 1): LCMS: 310 [M+1]⁺.

### Step 2c. Ethyl 7-(2-(1-(tetrahydro-2H-pyran-2-yl)-3-((E)-2-(pyridin-2-yl)vinyl)-1H-indazol-6-ylthio)benzamido)heptanoate (Compound 111-16)

The title compound 111-16 was prepared (620 mg, 94%) from compound 110-16 (400 mg, 1.29 mmol) and 106 (460 mg, 1.08 mmol) using a procedure similar to that described for compound 111-15 (Example 1): LCMS: 613 [M+1]⁺.

### Step 2d. Ethyl 7-(2-(3-((E)-2-(pyridin-2-yl)vinyl)-1H-indazol-6-ylthio)benzamido) heptanoate (Compound 112-16)

The title compound 112-16 was prepared (360 mg, 69%) from compound 111-16 (600 mg, 0.98 mmol) using a procedure similar to that described for compound 112-15 (Example 1): LCMS: 529 [M+1]⁺.

### Step 2e. N-(6-(Hydroxycarbamoyl)hexyl)-2-(3-((E)-2-(pyridin-2-yl)vinyl)-1H-indazol-6-ylthio)benzamide (Compound 16)

The title compound 16 was prepared (306 mg, 59%) from compound 112-16 (352 mg, 0.67 mmol) using a procedure similar to that described for compound 15 (Example 1): LCMS: 516 [M+1]⁺; ¹H NMR (DMSO-d₆): δ 1.23-1.30 (m, 4H), 1.32-1.36 (m, 4H), 1.94 (t, J=7.2 Hz, 2H), 3.21-3.25 (m, 2H), 7.06-7.08 (m, 1H), 7.16-7.20 (m, 1H), 7.26-7.33 (m, 3H), 7.46-7.49 (m, 1H), 7.54-7.65 (m, 3H), 7.78-7.84 (m, 1H), 7.94 (d, J=16.2 Hz, 1H), 8.19 (d, J=8.7 Hz, 1H), 8.43 (t, J=5.4 Hz, 1H), 8.60-8.62 (m, 1H).

TABLE 14-A
SECTION 14:

| | |
|---|---|
| | (XVII) |
| 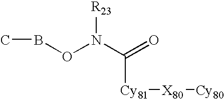 | |

| Compound # | Structure |
|---|---|
| 1 | 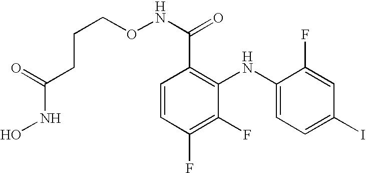 |
| 2 | 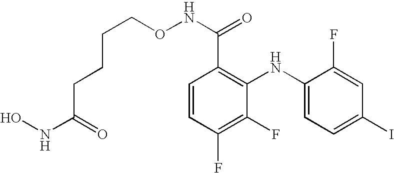 |
| 3 | 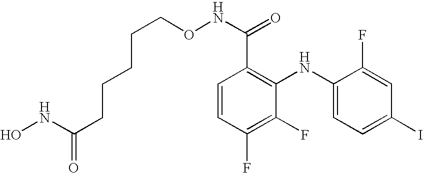 |
| 4 | 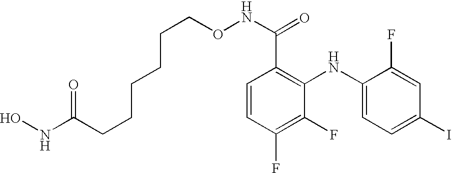 |
| 5 | 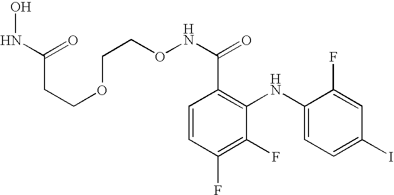 |
| 6 | 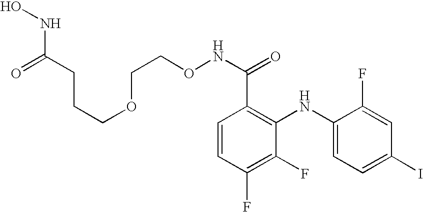 |
| 7 | 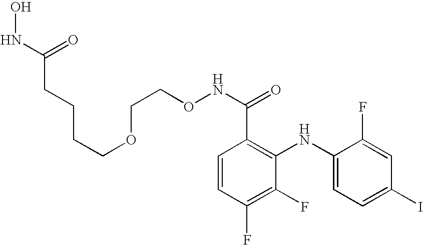 |
| 8 | 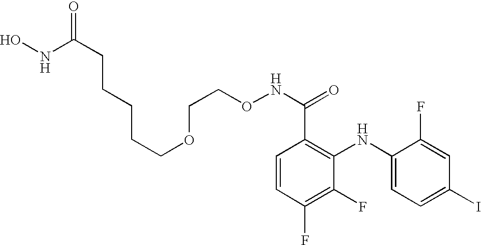 |
| 9 | 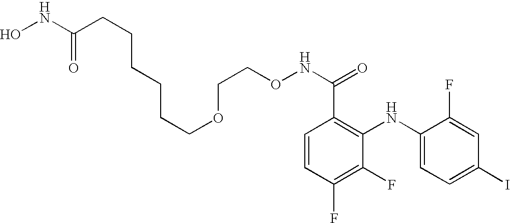 |
| 10 | 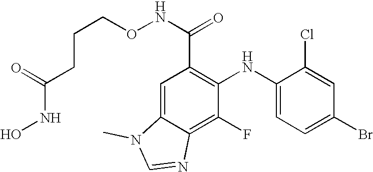 |
| 11 | 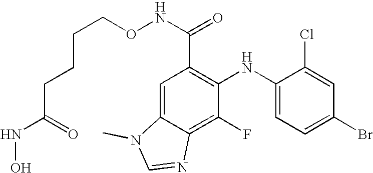 |
| 12 | 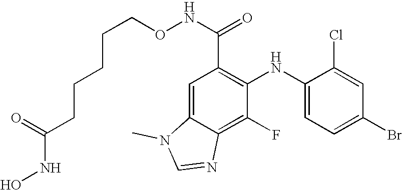 |
| 13 | 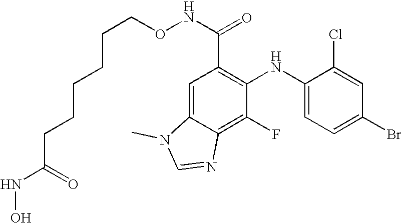 |
| 14 | 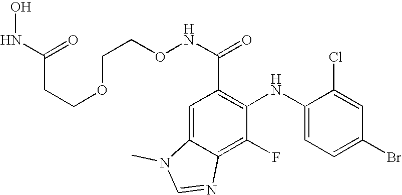 |
| 15 | 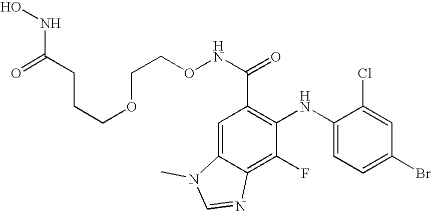 |
| 16 | 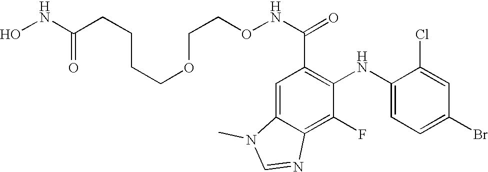 |
| 17 | 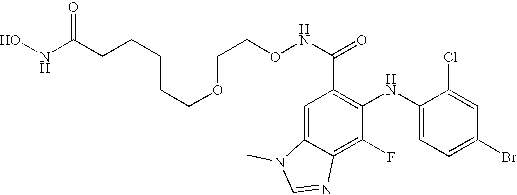 |
| 18 | 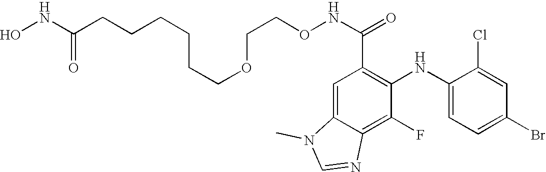 |

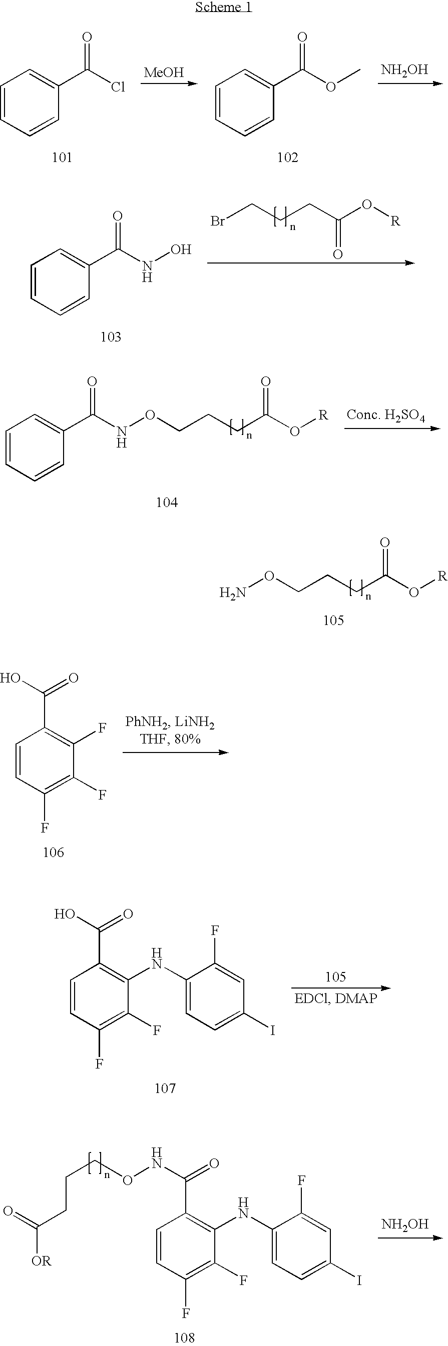

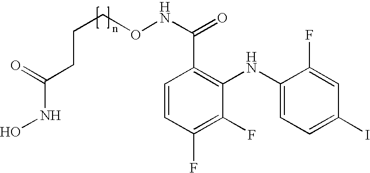

### Example 1

### Preparation of 3,4-difluoro-2-(2-fluoro-4-iodophenylamino)-N-(4-(hydroxyamino)-4-oxobutoxy)benzamide (Compound 1)

### Step 1a. Methyl benzoate (Compound 102)

The compound benzoyl chloride 101 (140 g, 1 mol) was added methanol (100 mL) at 0° C. The mixture was stirred at 0° C. for 5 min. and was concentrated to afford the compound methyl benzoate as a yellow oil (135 g, 99%): LC-MS: 137 [M+1]⁺.

### Step 1b. N-hydroxybenzamide (Compound 103-1)

Preparation of a solution of hydroxylamine in methanol: hydroxylamine hydrochloride (107.41 g, 1.56 mol) was dissolved in methanol (552 mL) to form solution A. Potassium hydroxide (129.03 g, 2.30 mol) was dissolved in methanol (322 mL) to Form solution B. Solution A was cooled to 0° C., and solution B was added into solution A dropwise. The mixture was stirred for 30 minutes at 0° C. The precipitate was filtered off and the filtrate formed a solution of hydroxylamine in methanol.

Methyl benzoate 102 (27.2 g, 0.2 mol) was dissolved in above solution of hydroxylamine in methanol (874 mL). The mixture was stirred at room temperature for 30 min. and was then adjusted to PH 7 with acetic acid. The mixture was concentrated to give a residue which was washed with water to afford compound 103 as a white solid (25 g, 91%). LC-MS: 138 [M+1]⁺.

### Step 1c. Ethyl 4-(benzamidooxy)butanoate (Compound 104-1)

Compound 103 (6.9 g, 50 mmol) was dissolved in DMF (100 mL), NaH (2.4 g, 60 mmol) was added into it at 0° C. The mixture was stirred at 0° C. for a few minutes and ethyl 4-bromobutanoate (9.7 g, 50 mmol) was added and the mixture was stirred at room temperature for 3 h. DMF was removed by evaporation and the residue was dissolved in CH₂Cl₂, washed with water and brine, dried with anhydrous Na₂SO₄, concentrated to give compound 104-1 as a yellow oil (2.3 g, 18%): LCMS: 252 [M+1]⁻.

### Step 1d. Methyl 4-(aminooxy)butanoate sulfate (Compound 105-1)

To a solution of compound 104-1 (2.3 g, 9 mmol) in methanol (30 mL) was added concentrated H₂SO₄ (0.898 g, 9 mmol). The mixture was stirred at 40° C. overnight. The methanol was removed and the residue was diluted with ethyl acetate, washed with water and brine, dried and concentrated to afford compound 105-1 (0.693 g, 33%). LCMS: 134 [M+1].

### Step 1e. 3,4-Difluoro-2-(2-fluoro-4-iodophenylamino)benzoic acid (Compound 107)

A solution 2-fluoro-4-iodoaniline (10 g, 0.057 mol) and 2,3,4-trifluorobenzoic acid 106 (13.5 g, 0.057 mol) was prepared and a portion (about 5%) of this solution was added to a stirring slurry of lithium amide (4.35 g, 0.182 mol) in 40 mL THF at 50-55° C. After about 15-30 min, an exotherm followed by gas release and color change were observed. The remaining portion of the solution was added slowly over 1-2 hours. Then, maintaining temperatures within 45-55° C. The mixture was stirred until the reaction was deemed complete (by LC-MS). The final mixture was then cooled to 20-25° C. and transferred to another reactor containing 6 N hydrochloric acid (47 mL) followed by 25 mL acetonitrile, stirred, and the bottom aqueous phase was discarded after treatment with 40 mL 50% sodium hydroxide solution. The organic phase was concentrated under reduced pressure and purified by column chromatography using CH₂Cl₂/MeOH (15/1) as eluent to yield compound 107 as a brown solid (15.9 g, 71% yield): LCMS: 394 [M+1]⁺; ¹H NMR (DMSO-d₆): δ 13.735 (s, 1H), 9.144 (s, 1H), 7.794 (m, 1H), 7.617 (m, 1H), 7.412 (m, 1H), 7.096 (m, 1H), 6.827 (m, 1H).

### Step 1f. Methyl 4-(3,4-difluoro-2-(2-fluoro-4-iodophenylamino)benzamidooxy)butanoate (Compound 108-1)

The mixture of the compound 107 (1.179 g, 3 mmol), EDCI HCl (0.86 g, 4.5 mmol), HOBt (0.61 g, 4.5 mmol), DIPEA (1.55 g, 12 mmol) and methyl 4-(aminooxy)butanoate sulfate 105 (0.693 g, 3 mmol) was stirred at 50° C. for 16 h. The mixture was diluted with ethyl acetate, washed with water and brine, dried over anhydrous Na₂SO₄ and concentrated to afford compound 108-1 as an oil (367 mg, 24%). LCMS: 509 [M+1]⁺.

### Step 1g. 3,4-Difluoro-2-(2-fluoro-4-iodophenylamino)-N-(4-(hydroxyld-amino)-4-oxobutoxy)benzamide (Compound 1)

Preparation of a solution of hydroxylamine in methanol: hydroxylamine hydrochloride (4.67 g, 67 mmol) was dissolved in methanol (24 mL) to form solution A. Potassium hydroxide (5.61 g, 100 mmol) was dissolved in methanol (14 mL) to form solution B. The solution A was cooled to 0° C., and solution B was added into solution A dropwise. The mixture was stirred for 30 minutes at 0° C. and the precipitate was filtered off. The filtrate formed the solution of hydroxylamine in methanol.

To a flask containing compound 108-1 (367 mg, 0.722 mmol) was added the solution of hydroxylamine in methanol (5.0 mL). The mixture was stirred at room temperature for 1 hour and was adjusted to PH 7 using acetic acid. The mixture was concentrated to give a residue which was washed with water to afford the product 1 as a solid (107 mg, 29% yield): LC-MS: 510 [M+1]; ¹H NMR (DMSO-d₆): δ 10.350 (s, 1H), 8.681 (s, 1H), 7.558 (d, J=9.0 Hz, 1H), 7.364 (m, 2H), 7.148 (m, 1H), 6.641 (m, 1H), 3.676 (t, J=6.1 Hz, 2H), 2.043 (m, 2H), 1.763 (m, 2H).

### Example 2

### Preparation of 3,4-difluoro-2-(2-fluoro-4-iodophenylamino)-N-(5-(hydroxyamino)-5-oxopentyloxy)benzamide (Compound 2)

### Step 2a. Methyl 5-(benzamidooxy)pentanoate (Compound 104-2)

The title compound 104-2 was prepared (2.74 g, 22%) from compound 103 (4.691 g, 34 mmol), NaH (1.632 g, 40.8 mmol) and methyl 5-bromopentanoate (6.63 g, 34 mmol) using a procedure similar to that described for compound 104-1 (Example 1): LCMS: 252 [M+1]⁺.

### Step 2b. Methyl 5-(aminooxy)pentanoate (Compound 105-2)

The title compound 105-2 was prepared (1.015 g, 63%) from compound 104-2 (2.74 g, 11 mmol) and concentrated H₂SO₄ (1.126 g, 11 mmol) using a procedure similar to that described for compound 105-1 (Example 1): LCMS: 148 [M+1]⁺.

### Step 2c. Methyl 5-(3,4-difluoro-2-(2-fluoro-4-iodophenylamino)benzami-dooxy)pentanoate (Compound 108-2)

The title compound 108-2 was prepared (988 mg, 55%) from compound 107 (1.357 g, 3.45 mmol), EDCI HCl (0.99 g, 5.18 mmol), HOBt (0.699 g, 5.18 mmol), DIPEA (1.337 g, 10.35 mmol) and methyl 5-(aminooxy)pentanoate 105-2 (0.508 g, 3.45 mmol) using a procedure similar to that described for compound 108-1 (Example 1): LCMS: 522 [M+1].

### Step 2d. 3,4-Difluoro-2-(2-fluoro-4-iodophenylamino)-N-(5-(hydroxylamino)-5-oxopentyloxy)benzamide (Compound 2)

The title compound 2 was prepared (119 mg, 45%) from compound 108-2 (261 mg, 0.5 mmol) and freshly prepared hydroxylamine in methanol (5.0 mL) using a procedure similar to that described for compound 1 (Example 1): LC-MS: 524 [M+1]; ¹H NMR (DMSO-d₆): δ 11.733 (s, 1H), 10.390 (s, 1H), 8.841 (s, 1H), 8.654 (s, 1H), 7.581 (m, 1H), 7.384 (m, 2H), 7.186 (m, 1H), 6.664 (m, 1H), 3.778 (m, 2H), 1.972 (t, J=6.0 Hz, 2H), 1.550 (m, 4H).

### Example 3

### Preparation of 3,4-difluoro-2-(2-fluoro-4-iodophenylamino)-N-(6-(hydroxyamino)-6-oxohexyloxy)benzamide (Compound 3)

### Step 3a. Ethyl 6-(benzamidooxy)hexanoate (Compound 104-3)

The title compound 104-3 was prepared (0.761 g, 12%) from compound 103 (3.179 g, 23 mmol), NaH (1.38 g, 34.5 mmol) and ethyl 6-bromohexanoate (5.114 g, 23 mmol) using a procedure similar to that described for compound 104-1 (Example 1): LCMS: 279 [M+1]⁺.

### Step 3b. Methyl 6-(aminooxy)hexanoate (Compound 105-3)

The title compound 105-3 was prepared (0.362 g, 62%) from compound 104-3 (958 g, 3.43 mmol) and concentrated H₂SO₄ (354 g, 3.43 mmol) using a procedure similar to that described for compound 105-1 (Example 1): LCMS: 162 [M+1]⁺.

### Step 3c. Methyl 6-(3,4-difluoro-2-(2-fluoro-4-iodophenylamino)benzami-dooxy)hexanoate (Compound 108-3)

The title compound 108-3 was prepared (250 mg, 17%) from compound 107 (1.056 g, 2.69 mmol), EDCI HCl (0.77 g, 40.035 mmol), HOBt (0.545 g, 4.035 mmol), DIPEA (2.085 g, 16014 mmol) and methyl 6-(aminooxy)hexanoate 105-3 (0.696 g, 2.69 mmol) using a procedure similar to that described for compound 108-1 (Example 1): LCMS: 537 [M+1]⁺.

### Step 3d. 3,4-Difluoro-2-(2-fluoro-4-iodophenylamino)-N-(6-(hydroxylamino)-6-oxohexyloxy)benzamide (Compound 3)

The title compound 3 was prepared (50 mg, 20% yield) from compound 108-3 (250 mg, 0.47 mmol) and freshly prepared hydroxylamine in methanol (8.0 mL) using a procedure similar to that described for compound 1 (Example 1): LC-MS: 538 [M+1]⁺; ¹H NMR (DMSO-d₆): δ 11.750 (s, 1H), 10.352 (s, 1H), 8.742 (s, 1H), 8.673 (s, 1H), 7.580 (m, 1H), 7.374 (m, 2H), 7.199 (m, 1H), 7.660 (m, 1H), 3.764 (m, 2H), 1.942 (m, 2H), 1.521 (m, 4H), 1.297 (m, 2H).

### Example 4

### Preparation of 3,4-difluoro-2-(2-fluoro-4-iodophenylamino)-N-(7-(hydroxyamino)-7-oxoheptyloxy)benzamide (Compound 4)

### Step 4a. Ethyl 7-(benzamidooxy)heptanoate (Compound 104-4)

The title compound 104-4 was prepared (0.635 g, 22%) from compound 103 (1.38 g, 10 mmol), NaH (0.48 g, 12 mmol) and ethyl 6-bromohexanoate (2.37 g, 10 mmol) using a procedure similar to that described for compound 104-1 (Example 1): LCMS: 294 [M+1]⁺.

### Step 4b. Methyl 7-(aminooxy)heptanoate (Compound 105-4)

The title compound 105-4 was prepared (227 mg, 60%) from compound 104-4 (635 mg, 2.17 mmol) and concentrated H₂SO₄ (223.6 g, 2.17 mmol) using a procedure similar to that described for compound 105-1 (Example 1): LCMS: 176 [M+1]⁺.

### Step 4c. Methyl 7-(3,4-difluoro-2-(2-fluoro-4-iodophenylamino)benzam-idooxy)heptanoate (Compound 108-4)

The title compound 108-4 was prepared (178 mg, 25%) from compound 107 (501 mg, 1.27 mmol), EDCI HCl (364 mg, 1.905 mmol), HOBt (257 mg, 1.905 mmol), DIPEA (656 mg, 5.08 mmol) and methyl 7-(aminooxy)heptanoate 105-4 (223 mg, 1.27 mmol) using a procedure similar to that described for compound 108-1 (Example 1): LCMS: 551 [M+1]⁺.

### Step 4d. 3,4-Difluoro-2-(2-fluoro-4-iodophenylamino)-N-(7-(hydroxyl-amino)-7-oxoheptyloxy)benzamide (Compound 4)

The title compound 4 was prepared (89 mg, 54% yield) from compound 108-4 (178 mg, 0.3 mmol) and freshly prepared hydroxylamine in methanol (3.0 mL) using a procedure similar to that described for compound 1 (Example 1): LCMS: 552 [M+1]⁺; ¹H NMR (DMSO-d₆): δ 11.699 (s, 1H), 10.329 (s, 1H), 8.881 (s, 1H), 8.645 (s, 1H), 7.575 (m, 1H), 7.381 (m, 2H), 7.191 (m, 1H), 6.657 (m, 1H), 3.752 (t, J=6.3 Hz, 2H), 1.934 (t, J=7.2 Hz, 2H), 1.482 (m, 4H), 1.263 (m, 4H).

### Biological Assays:

As stated hereinbefore the derivatives defined in the present invention possess anti-proliferation activity. These properties may be assessed, for example, using one or more of the procedures set out below:

### (a) MEK Enzyme Assay

The activity of the compounds of the present invention may be determined by the following procedure. N-terminal 6 His-tagged MEK-1 (2-393) is expressed in E. coli and protein is purified by conventional methods (Ahn et al., Science 1994, 265, 966-970) and activated by Raf-1. The activity of MEK1 is assessed by measuring the incorporation of γ-³³P-phosphate from γ-³³P-ATP onto N-terminal His tagged, kinase mutated (K52R) ERK2, which is expressed in E. coli and is purified by conventional methods. The assay is carried out in 96-well polypropylene plate. The incubation mixture (100 μL) comprises of 20 mM Hepes, pH 7.4, 10 mM MgCl.sub.2, 1 mM EGTA, 0.02% Brij, 0.02 mg/ml BSA, 100 .mu.M Na-orthovanadate, 2 mM DTT, 0.5 nM MEK1, and 1 μM ERK2. Inhibitors are suspended in DMSO, and all reactions, including controls are performed at a final concentration of 1% DMSO. Reactions are carried in the presence of 1 μM ATP (with 0.5 μCi γ-³³P-ATP/well) and incubated at ambient temperature for 120 minutes. Equal volume of 25% TCA is added to stop the reaction and precipitate the proteins. Precipitated proteins are trapped onto glass fiber B filterplates, and excess labeled ATP washed off using a Tomtec MACH III harvestor. Plates are allowed to air-dry prior to adding 30 μL/well of Packard Microscint 20, and plates are counted using a Perkin Elmer TopCount. In this assay, compounds of the invention exhibited an IC50 of less than 50 micromolar.

### (b) Cellular ERK 1/2 Phosphorylation Assay

The MEK 1/2 inhibition properties of the compounds of the invention may be determined by the following in vitro cellular assay. Inhibition of basal ERK1/2 phosphorylation is determined by incubating cells with compound for 1 hour and quantifying the pERK signal on fixed cells and normalizing to total ERK signal. Materials and Methods: Malme-3M cells are obtained from ATCC and grown in RPMI-1640 supplemented with 10% fetal bovine serum. Cells are plated in 96-well plates at 15,000 cells/well and allowed to attach for 1-2 hours. Diluted compounds are then added at a final concentration of 1% DMSO. After 1 hour, cells are washed with PBS and fixed in 3.7% para-formaldehyde in PBS for 15 minutes. This is followed by washing in PBS/0.1% Triton X-100. Cells are blocked in Odyssey blocking buffer (LI-COR Biosciences) for at least 1 hour. Antibodies to phosphorylated ERK 1/2 (Cell Signaling #9106, monoclonal) and total ERK 12 (Santa Cruz Biotechnology #sc-94, polyclonal) are added to the cells and incubated for at least 1 hour. After washing with PBS/0.1% TritonX-100, the cells are incubated with fluorescently-labeled secondary antibodies (goat anti-rabbit IgG-IRDye800, Rockland and goat anti-mouse IgG-Alexa Fluor 680, Molecular Probes) for an additional hour. Cells are then washed and analyzed for fluorescence at both wavelengths using the Odyssey Infrared Imaging System (LI-COR Biosciences). Phosphorylated ERK signal is normalized to total ERK signal.

(c) An In Vitro Assay which Determines the Ability of a Test Compound to Inhibit HDAC Enzymatic Activity.

HDAC inhibitors were screened using an HDAC fluorimetric assay kit (AK-500, Biomol, Plymouth Meeting, Pa.). Test compounds were dissolved in dimethylsulphoxide (DMSO) to give a 20 mM working stock concentration. Fluorescence was measured on a WALLAC Victor 2 plate reader and reported as relative fluorescence units (RFU). Data were plotted using GraphPad Prism (v4.0a) and IC50's calculated using a sigmoidal dose response curve fitting algorithm. Each assay was setup as follows: Defrosted all kit components and kept on ice until use. Diluted HeLa nuclear extract 1:29 in Assay Buffer (50 mM Tris/Cl, pH 8.0, 137 mM NaCl, 2.7 mM KCl, 1 mM MgCl2). Prepared dilutions of Trichostatin A (TSA, positive control) and tested compounds in assay buffer (5× of final concentration). Diluted Fluor de Lys™ Substrate in assay buffer to 100 uM (50 fold=2× final). Diluted Fluor de Lys™ developer concentrate 20-fold (e.g. 50 μl plus 950 μl Assay Buffer) in cold assay buffer. Second, diluted the 0.2 mM Trichostatin A 100-fold in the 1× Developer (e.g. 10 μl in 1 ml; final Trichostatin A concentration in the 1× Developer=2 μM; final concentration after addition to HDAC/Substrate reaction=1 μM). Added Assay buffer, diluted trichostatin A or test inhibitor to appropriate wells of the microtiter plate. Added diluted HeLa extract or other HDAC sample to all wells except for negative controls. Allowed diluted Fluor de Lys™ Substrate and the samples in the microtiter plate to equilibrate to assay temperature (e.g. 25 or 37° C. Initiated HDAC reactions by adding diluted substrate (25 μl) to each well and mixing thoroughly. Allowed HDAC reactions to proceed for 1 hour and then stopped them by addition of Fluor de Lys™ Developer (50 μl). Incubated plate at room temperature (25° C.) for 10-15 min. Read samples in a microtiter-plate reading fluorimeter capable of excitation at a wavelength in the range 350-380 nm and detection of emitted light in the range 440-460 nm.

The following TABLE 14-B lists compounds representative of the invention and their activity in HDAC and MEK assays. In these assays, the following grading was used: I≧10 μM, 10 μM>II>1 μM, 1 μM>III>0.1 μM, and IV≦0.1 μM for IC₅₀.

TABLE 14-B

| Compound No. | HDAC | MEK-1 |
|---|---|---|
| 1 | II | III |
| 2 | III | |
| 3 | IV | III |
| 4 | IV | |

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A multi-functional small molecule compound wherein one functionality is capable of inhibiting histone deacetylases (HDAC) and the other functionality is capable of inhibiting at least one other cellular or molecular pathway involved in aberrant cell proliferation, differentiation or survival.

2. A compound of claim 1, wherein said tumor cellular or molecular pathway is selected from tyrosine kinases, serine/threonine kinases, DNA methyl transferases, proteosome, matrix metalloproteinase, farnesyl transferase, heat-shock proteins, and apoptosis.

3. A compound of claim 1, wherein said tumor cellular or molecular pathway is EGFR, ErbB2, ErbB3, ErbB4, HER-2, VEGFR-1, VEGFR-2, VEGFR-3Flt-3, c-kit, Abl, JAK, PDGFR-a, PDGFR-b, IGF-IR, c-Met, FGFR1, FGFR3, FGFR4, c-Ret, Src, Lyn, Yes, PKC, CDK, Erk, Merk, PI3K-Akt, mTOR, Raf, CHK, Aurora, HSP90, TRAILR, caspases, IAPs, Bcl-2, Survivin, MDM2, MDM4.

4. A compound represented by formula (I),
A-B—C (I)
or its geometric isomers, enantiomers, diastereomers, racemates, pharmaceutically acceptable salts, prodrugs and solvates thereof, where A is a pharmacophore of an anti-cancer agent capable of inhibiting at least one cellular or molecular pathway involved in the aberrant cell proliferation, differentiation or survival; B is a linker and C is a zinc-binding moiety.

5. A compound of claim 4, wherein the anticancer agent is selected from inhibitors of EGFR, ErbB2, ErbB3, ErbB4, HER-2, VEGFR-1, VEGFR-2, VEGFR-3Flt-3, c-kit, Abl, JAK, PDGFR-a, PDGFR-b, IGF-IR, c-Met, FGFR1, FGFR3, FGFR4, c-Ret, Src, Lyn, Yes, PKC, CDK, Erk, Merk, PI3K-Akt, mTOR, Raf, CHK, Aurora, HSP90, TRAILR, caspases, IAPs, Bcl-2, Survivin, MDM2, MDM4.

6. A compound of claim 4, wherein C is a zinc-binding moiety is selected from the group consisting of: 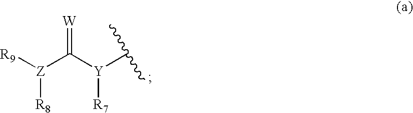
where W is O or S; Y is absent, N or CH; Z is N or CH; R₇ and R₉ are independently hydrogen, OR′, aliphatic or substituted aliphatic, wherein R′ is hydrogen, acyl, aliphatic or substituted; provided that if R₇ and R₉ are both present, then one of R₇ or R₉ must be OR′ and if Y is absent, R₉ must be OR; and R₈ is hydrogen, acyl, aliphatic, substituted aliphatic; 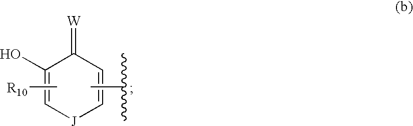
where W is O or S; J is O, NH, or NCH₃; and R₁₀ is hydrogen or lower alkyl; 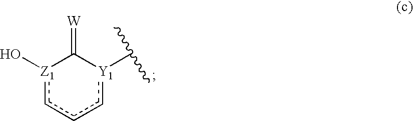
where W is O or S; Y₁ and Z₁ are independently N, C or CH; and 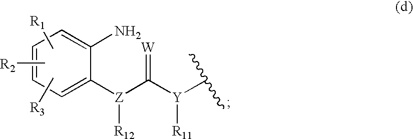
where Z, Y, and W are as previously defined; R₁₁ R₁₂ are independently selected from hydrogen or aliphatic; R₁, R₂ and R₃ are independently selected from hydrogen, hydroxy, amino, halogen, alkoxy, substituted alkoxy, alkylamino, substituted alkylamino, dialkylamino, substituted dialkylamino, substituted or unsubstituted alkylthio, substituted or unsubstituted alkylsulfonyl, CF₃, CN, NO₂, N₃, sulfonyl, acyl, aliphatic, substituted aliphatic, aryl, substituted aryl, heteroaryl, substituted heteroaryl, heterocyclic and substituted heterocyclic.

7. A compound of claim 6, wherein C is a zinc-binding moiety is selected from the group consisting of: 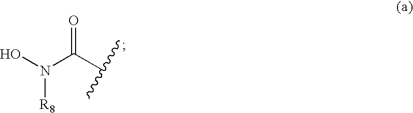
where R₈ is selected from hydrogen or lower alkyl; and 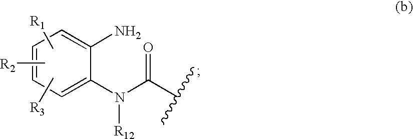
where R₁, R₂ and R₃ are independently selected from hydrogen, hydroxy, CF₃, NO₂, N₃, halogen, lower alkyl, lower alkoxy, lower alkylamino, alkoxyalkoxy, alkylaminoalkoxy phenyl, thiophenyl, furanyl, pyrazinyl, substituted pyrazinyl, and morpholino; and R₁₂ is selected from hydrogen or lower alkyl.

8. A compound of claim 4, wherein B is a direct bond or straight- or branched-, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, or alkynylhereroaryl, which one or more methylenes can be interrupted or terminated by O, S, S(O), SO₂, N(R₈), C(O), substituted or unsubstituted aryl, substituted or unsubstituted heteroaryl, substituted or unsubstituted heterocyclic, where R₈ is previously defined in claim 6.

9. A compound of claim 4, wherein B is a straight chain, alkyl, alkenyl, alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, or alkynylhereroaryl,. One or more methylenes can be interrupted or terminated by —O—, —N(R₈)—, —C(O)—, —C(O)N(R₈)—, or —C(O)O—, where R₈ is previously defined in claim 6.

10. A compound of claim 4, wherein B is between 1-24 atoms, preferably 4-24 atoms, preferably 4-18 atoms, more preferably 4-12 atoms, and most preferably about 4-10 atoms.

11. A compound of claim 4, wherein B is selected from straight chain C1-C10 alkyl, C1-C10 alkenyl, C1-C10 alkynyl, C1-C10 alkoxy, alkoxyC1-C10alkoxy, C1-C10 alkylamino, alkoxyC1-C10alkylamino, C1-C10 alkylcarbonylamino, C1-C10 alkylaminocarbonyl, aryloxyC1-C10alkoxy, aryloxyC1-C10alkylamino, aryloxyC1-C10alkylamino carbonyl, C1-C10-alkylaminoalkylaminocarbonyl, C1-C10 alkyl(N-alkyl)aminoalkyl-aminocarbonyl, alkylaminoalkylamino, alkylcarbonylaminoalkylamino, alkyl(N-alkyl)aminoalkylamino, (N-alkyl)alkylcarbonylaminoalkylamino, alkylaminoalkyl, alkylaminoalkylaminoalkyl, alkylpiperazinoalkyl, piperazinoalkyl, alkylpiperazino, alkenylaryloxyC1-C10alkoxy, alkenylarylaminoC1-C10alkoxy, alkenylaryllalkylaminoC1-C10alkoxy, alkenylaryloxyC1-C10alkylamino, alkenylaryloxyC1-C10alkylaminocarbonyl, piperazinoalkylaryl, heteroarylC1-C10alkyl, heteroarylC2-C10alkenyl, heteroarylC2-C10alkynyl, heteroarylC1-C10alkylamino, heteroarylC1-C10alkoxy, heteroaryloxyC1-C10alkyl, heteroaryloxyC2-C10alkenyl, heteroaryloxyC2-C10alkynyl, heteroaryloxyC1-C10alkylamino, heteroaryloxyC1-C10alkoxy.

12. A compound of claim 4, wherein C is a zinc-binding moiety is selected from the group consisting of: 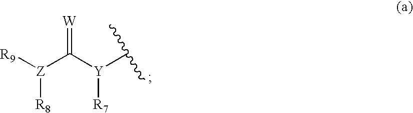
where W is O or S; Y is absent, N, or CH; Z is N or CH; R₇ and R₉ are independently hydrogen, hydroxy, aliphatic group, provided that if R₇ and R₉ are both present, one of R₇ or R₉ must be hydroxy and if Y is absent, R₉ must be hydroxy; and R₈ is hydrogen or aliphatic group; 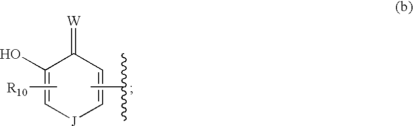
where W is O or S; J is O, NH or NCH₃; and R₁₀ is hydrogen or lower alkyl; 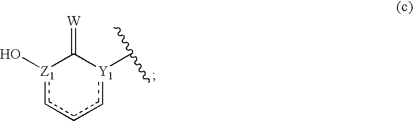
where W is O or S; Y₁ and Z₁ are independently N, C or CH; and 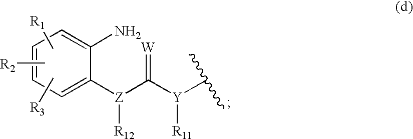
where Z, Y, and W are as previously defined; R₁₁ R₁₂ are independently selected from hydrogen or aliphatic; R₁, R₂ and R₃ are independently selected from hydrogen, hydroxy, amino, halogen, alkoxy, alkylamino, dialkylamino, CF₃, CN, NO₂, sulfonyl, acyl, aliphatic, substituted aliphatic, aryl, substituted aryl, heteroaryl, substituted heteroaryl, heterocyclic, and substituted heterocyclic.

13. A compound of claim 4, wherein C is a zinc-binding moiety is selected from the group consisting of: 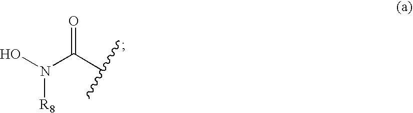
where R₈ is selected from hydrogen or lower alkyl; and 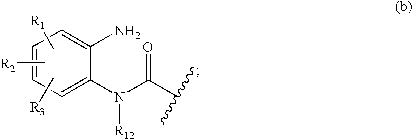
where R₁, R₂ and R₃ are independently selected from hydrogen, hydroxy, CF₃, NO₂, halogen, lower alkyl, lower alkoxy, lower alkylamino, alkoxyalkoxy, alkylaminoalkoxy, phenyl, thiophenyl, furanyl, pyrazinyl, substituted pyrazinyl, and morpholino; and R₁₂ is selected from hydrogen or lower alkyl.

14. A compound of claim 4, wherein B is a direct bond or straight- or branched-, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, which one or more methylenes can be interrupted or terminated by O, S, S(O), SO₂, N(R₈), C(O), substituted or unsubstituted aryl, substituted or unsubstituted heteroaryl, substituted or unsubstituted heterocyclic; where R₈ is previously defined in claim 12.

15. A compound of claim 4, wherein B is a straight chain alkyl, alkenyl, alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, or alkynylheterocyclylalkynyl, where one or more methylenes can be interrupted or terminated by —O—, —N(R₈)—, —C(O)—, —C(O)N(R₈)—, or —C(O)O—, where R₈ is previously defined in claim 12.

16. A compound of claim 4, wherein B is selected from straight chain C1-C10 alkyl, C1-C10 alkenyl, C1-C10 alkynyl, C1-C10 alkoxy, alkoxyC1-C10alkoxy, C1-C10 alkylamino, alkoxyC1-C10alkylamino, C1-C10 alkylcarbonylamino, C1-C10 alkylaminocarbonyl, aryloxyC1-C10alkoxy, aryloxyC1-C10alkylamino, aryloxyC1-C10alkylamino carbonyl, C1-C10-alkylamino-alkylaminocarbonyl, C1-C10 alkyl(N-alkyl)aminoalkylaminocarbonyl, alkylaminoalkylamino, alkylcarbonylaminoalkylamino, alkyl(N-alkyl)aminoalkylamino, (N-alkyl)alkylcarbonylaminoalkylamino, alkylaminoalkyl, alkylaminoalkylaminoalkyl, alkylpiperazinoalkyl, piperazinoalkyl, alkylpiperazino, alkenylaryloxyC1-C10alkoxy, alkenylarylaminoC1-C10alkoxy, alkenylaryllalkylaminoC1-C10alkoxy, alkenylaryloxyC1-C10alkylamino, alkenylaryloxyC1-C10alkylaminocarbonyl and piperazinoalkylaryl.

17. A pharmaceutical composition comprising as an active ingredient a compound of claim 4 and a pharmaceutical acceptable carrier.

18. A method of treating a cell proliferative disorder in a subject in need thereof, the method comprising administering to the subject a therapeutically effective amount of the pharmaceutical composition of claim 17.

19. A method of treating and/or preventing immune response or immune-mediated responses and diseases in a subject in need thereof, the method comprising administering to the subject a therapeutically effective amount of the pharmaceutical composition of claim 17.

20. A method of treating neurodegenerative diseases in a subject in need thereof, the method comprising administering to the subject a therapeutically effective amount of the pharmaceutical composition of claim 17.
